(19) United States
(12) Patent Application Publication
(10) Pub. No.: US 2023/0298696 A1
(43) Pub. Date: Sep. 21, 2023

(54) BIOMARKERS AND METHODS OF SELECTING AND USING THE SAME

(71) Applicant: THE REGENTS OF THE UNIVERSITY OF CALIFORNIA, Oakland, CA (US)

(72) Inventors: Marina SIROTA, Belmont, CA (US); Dmitry RYCHKOV, Fremont, CA (US)

(21) Appl. No.: 18/017,650

(22) PCT Filed: Jul. 23, 2021
(86) PCT No.: PCT/US2021/043033

§ 371 Date: Jan. 23, 2023

Related U.S. Application Data
(60) Provisional application No. 63/056,532, filed on Jul. 24, 2020.

(51) Int. Cl. G16B 25/10; G16B 40/20; C12Q 1/6876; G06N 20/00

(52) U.S. Cl. CPC G16B 25/10; G16B 40/20; C12Q 1/6876; G06N 20/00; C12Q 2600/158

(57) ABSTRACT

The disclosure generally relates to methods of selecting a biomarker associated with a disorder or disease, and computer program products and systems for performing such methods. The disclosure further relates to biomarkers for rheumatoid arthritis and methods of use such biomarkers.

### CROSS-REFERENCE TO RELATED APPLICATIONS

This application claims the benefit of U.S. Application No. 63/056,532, filed on Jul. 24, 2020, the contents of which are hereby incorporated by reference in their entirety.

### STATEMENT REGARDING FEDERALLY SPONSORED RESEARCH

This invention was made with government support under grant No. P30 AR070155 awarded by The National Institutes of Health. The government has certain rights in the invention.

### FIELD OF INVENTION

The disclosure generally relates to methods of selecting a biomarker associated with a disorder or disease, and computer program products and systems for performing such methods. Also provided are biomarkers and methods for generating scores useful for diagnosing rheumatoid arthritis (RA) and/or assessing RA disease activity in subjects previously diagnosed with RA.

### BACKGROUND

Over the past decade, advances in genomic sequencing technology have greatly contributed to our understanding of diseases, such as inflammatory diseases, and informed development of effective therapeutics. Transcriptomics provides a lens into the specific genes over- or under-expressed in a disease providing insight into cellular responses. Given the numerous transcriptomic datasets that have been generated and made publicly available, there are now opportunities to combine these datasets in a meta-analytic fashion for unbiased computational biomarker discovery. Meta-analysis is a systematic approach to combine and integrate cohorts to study a disease condition which provides enhanced statistical power due to a higher number of samples when combined. Additionally, it provides an opportunity of leveraging all the disease heterogeneity combined from multiple smaller studies across diverse populations what allows creating a robust signature and better recognizing direct disease drivers as well as disease subtyping and patient stratification. Moreover, integrating datasets generated from the multiple target tissues within a given disease further strengthens the associations identified. This approach has been successfully applied to the study of antineutrophil cytoplasmic antibody (ANCA)-associated vasculitis, dermatomyositis and systemic lupus erythematosus. These large datasets also present an opportunity to apply novel machine learning approaches that were not previously beasible computationally allowing for interrogation of the data with new and unbiased approaches.

Rheumatoid arthritis (RA) is a systemic inflammatory condition characterized by a symmetric and destructive distal polyarthritis. Undiagnosed and untreated, RA can progress to severe joint damage, involve other organ systems, and predispose individuals to cardiovascular disease. While our understanding of disease pathogenesis has greatly improved, and the number of available, effective therapeutics has significantly increased, there remains significant barriers to caring for patients with RA, and they continue to suffer from the morbidity and mortality associated with the disease. There remains an urgent need to develop objective biomarkers for the early diagnosis and prompt initiation of disease-modifying therapy during the so-called “window of opportunity.” Additionally, clinicians need tests to help accurately assess disease activity or treatment targets in order to adjust therapy appropriately. Identification of biomarkers would greatly add to clinicians' existing toolset used to evaluate patients with RA helping to improve outcomes and alleviate the suffering caused by this prevalent disease.

Multiple studies attempted to identify RA transcriptomics signature in blood and in synovial tissue separately or in a cross-tissue analysis. The integrative meta-analysis studies normally combined a few datasets from each tissue to identify an overlap of dysregulated genes and to recognize similarities and differences in disease pathways in both tissues. While this type of approach allows better understanding of the disease, a corresponding set of biomarkers is often redundant and requires extensive prioritization analysis and validation. Thus, more rigorous approaches for biomarkers search with a built-in prioritization procedure are still in unmet need in RA.

### SUMMARY OF EMBODIMENTS

The disclosure relates to a method of selecting a biomarker associated with a disorder or disease, the method comprising: a) creating a test data set and a training data set from an input set of data, wherein the input set of data comprises gene expression profiles of subjects having the disorder or disease and of control subjects; b) identifying a significant expression profile using a statistical test; c) evaluating expression performance of the significant expression profile by applying a machine learning methods to create a performance algorithm; and d) selecting a biomarker associated with the disorder or disease based on a threshold of the performance algorithm.

The disclosure also relates to a method of selecting a biomarker associated with a disorder or disease, the method comprising: a) creating a test data set and a training data set from an input set of data, wherein the input set of data comprises gene expression profiles of subjects having the disorder or disease and control subjects; b) identifying one or a plurality of significant expression profiles correlated with the disorder or disease in the training data set using a statistical test; c) evaluating expression performance of each of the significant expression profiles by applying one or a plurality of machine learning methods to create a performance algorithm; d) testing the performance algorithm on the test data set; e) selecting a high performing expression profile corresponding to at least one biomarker based upon a first threshold of the performance algorithm; f) testing the high performing expression profile selected in step e) with a dataset, said dataset being independent from the input set of data; and g) selecting a biomarker associated with the disorder or disease based on a second threshold of the performance algorithm. In some embodiments, the method further comprises repeating step a) through d) from at least about 2 to about 100 times. In some embodiments, the method further comprises one or a combination of: (i) compiling data from a provider; (ii) assessing quality control; and/or (iii) data processing normalizing prior to performing step a). In some embodiments, the method further comprises eliminating an expression profile of a particular gene, locus or nucleic acid sequence from being a biomarker if the expression profile performance of said particular gene, locus or nucleic acid sequence is inconsistent between different datasets or tissue types.

In some embodiments, the test data set and the training data set used in the disclosed method comprise a random spilt of the input set of data in a ratio of about 1:3. In some embodiments, the test data set and the training data set comprise a random spilt of the input set of data in a ratio of about 1:4. In some embodiments, the test data set and the training data set comprise a random spilt of the input set of data in a ratio of about 1:5.

In some embodiments, the statistical test used in step b) of the disclosed method to identify the set of significant expression profiles comprises linear models for microarray data (limma) with a p-value less than about 0.05. In some embodiments, the one or plurality of machine learning methods used in step c) of the disclosed method comprise a linear regression, a logistic regression, a decision tree, an elastic net and/or a random forest. In some embodiments, the one or plurality of machine learning methods used in step c) comprise a logistic regression model. In some embodiments, the performance algorithm created by the disclosed method is validated on the test data set using area under receiver operating characteristic (AUROC) curve wherein the AUROC is from about 0.5 to about 0.9.

Thresholds, which are used herein, to describe the value above which or under which a selection determination is made by the processor or the user of the disclosed system for purposes of executing the steps with selection criteria. In some embodiments, the first threshold used in the disclosed method is a mean AUROC higher than about 0.6. In some embodiments, the first threshold is a mean AUROC higher than about 0.7. In some embodiments, the first threshold is a mean AUROC equal to or higher than about 0.67.

In some embodiments, the second threshold used in the disclosed method is a mean AUROC equal to or higher than about 0.8. In some embodiments, the second threshold is a mean AUROC is equal to or higher than about 0.9.

In some embodiments, the input set of data used in the disclosed method comprises normalized microarray data. In some embodiments, the input set of data comprises normalized RNA-seq data. In some embodiments, the input set of data used in the disclosed method comprises normalized microarray data and normalized RNA-seq data. In some embodiments, the input set of data comprises expression profiles from a single tissue. In some embodiments, the input set of data comprises expression profiles from at least two different tissue types.

In some embodiments, the disorder or disease with which the biomarker selected by the disclosed method is arthritis. In some embodiments, the disorder or disease with which the biomarker selected by the disclosed method is rheumatoid arthritis.

Also contemplated in the disclosure is the biomarker selected by any of the disclosed methods.

The disclosure further relates to a computer program product encoded on a computer-readable storage medium comprising instructions for executing any of the above disclosed methods for selecting a biomarker associated with a disorder or disease. Also provided is a system comprising the disclosed computer program product and a processor operable to execute programs, and/or a memory associated with the processor.

The disclosure also relates to a system for selecting a biomarker associated with a disorder or disease, the system comprising: a) a processor operable to execute programs; b) a memory associated with the processor; c) a database associated with said processor and said memory; and d) a program product stored in the memory and executable by the processor, the program being operable for executing any of the above disclosed methods for selecting a biomarker associated with a disorder or disease.

The disclosure also relates to a composition comprising nucleic acid sequences complementary to one or a combination of: TNFAIP6, S100A8, TNFSF10, DRAM1, LY96, QPCT, KYNU, ENTPD1, CLIC1, ATP6V0E1, HSP90AB1, NCL, and CIRBP. In some embodiments, the disclosed composition comprises:
a) the nucleic acid sequence is complementary to a nucleic acid sequence comprising at least about 70% sequence identity to SEQ ID NO: 1;
b) the nucleic acid sequence is complementary to a nucleic acid sequence comprising at least about 70% sequence identity to SEQ ID NO: 3, SEQ ID NO: 5, SEQ ID NO: 7, SEQ ID NO: 9, and/or SEQ ID NO: 11;
c) the nucleic acid sequence is complementary to a nucleic acid sequence comprising at least about 70% sequence identity to SEQ ID NO: 13;
d) the nucleic acid sequence is complementary to a nucleic acid sequence comprising at least about 70% sequence identity to SEQ ID NO: 15, SEQ ID NO: 17 and/or SEQ ID NO: 19;
e) the nucleic acid sequence is complementary to a nucleic acid sequence comprising at least about 70% sequence identity to SEQ ID NO: 21 and/or SEQ ID NO: 23;
f) the nucleic acid sequence is complementary to a nucleic acid sequence comprising at least about 70% sequence identity to SEQ ID NO: 25;
g) the nucleic acid sequence is complementary to a nucleic acid sequence comprising at least about 70% sequence identity to SEQ ID NO: 27, SEQ ID NO: 29 and/or SEQ ID NO: 31;
h) the nucleic acid sequence is complementary to a nucleic acid sequence comprising at least about 70% sequence identity to SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47 and/or SEQ ID NO: 49;
i) the nucleic acid sequence is complementary to a nucleic acid sequence comprising at least about 70% sequence identity to SEQ ID NO: 51, SEQ ID NO: 53, and/or SEQ ID NO: 55;
j) the nucleic acid sequence is complementary to a nucleic acid sequence comprising at least about 70% sequence identity to SEQ ID NO: 57;
k) the nucleic acid sequence is complementary to a nucleic acid sequence comprising at least about 70% sequence identity to SEQ ID NO: 59;
l) the nucleic acid sequence is complementary to a nucleic acid sequence comprising at least about 70% sequence identity to SEQ ID NO: 61, SEQ ID NO: 63 and/or SEQ ID NO: 65; and
m) the nucleic acid sequence is complementary to a nucleic acid sequence comprising at least about 70% sequence identity to SEQ ID NO: 67, SEQ ID NO: 69, SEQ ID NO: 71, SEQ ID NO: 73, SEQ ID NO: 75 and/or SEQ ID NO: 77.
   In some embodiments, the disclosed composition comprises a combination of all of the nucleic acid sequences of a) through m) above.

In some embodiments, the disclosure provides a system comprising a solid support and one or a plurality of probes complementary to one or a plurality of biomarkers disclosed herein. In some embodiments, the one or plurality of probes are immobilized or absorbed onto the solid support. In some embodiments, the probes comprised in the disclosed system are complementary to one or a plurality of biomarkers chosen from a) through m) above.

The disclosure also relates to a system comprising a solid support and one or a plurality of antigen binding fragments specifically bind to one or a plurality of biomarkers disclosed herein. In some embodiments, the one or plurality of antigen binding fragments are immobilized or absorbed onto the solid support. In some embodiments, the antigen binding fragments comprised in the disclosed system bind specifically to one or a plurality of biomarkers chosen from:
a) a polypeptide comprising at least about 70% sequence identity to SEQ ID NO: 2;
b) a polypeptide comprising at least about 70% sequence identity to SEQ ID NO: 4, SEQ ID NO: 6, SEQ ID NO: 8, SEQ ID NO: 10 and/or SEQ ID NO: 12;
c) a polypeptide comprising at least about 70% sequence identity to SEQ ID NO: 14;
d) a polypeptide comprising at least about 70% sequence identity to SEQ ID NO: 16, SEQ ID NO: 18 and/or SEQ ID NO: 20;
e) a polypeptide comprising at least about 70% sequence identity to SEQ ID NO: 22 and/or SEQ ID NO: 24;
f) a polypeptide comprising at least about 70% sequence identity to SEQ ID NO: 26;
g) a polypeptide comprising at least about 70% sequence identity to SEQ ID NO: 28, SEQ ID NO: 30 and/or SEQ ID NO: 32;
h) a polypeptide comprising at least about 70% sequence identity to SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48 and/or SEQ ID NO: 50;
i) a polypeptide comprising at least about 70% sequence identity to SEQ ID NO: 52, SEQ ID NO: 54 and/or SEQ ID NO: 56;
j) a polypeptide comprising at least about 70% sequence identity to SEQ ID NO: 58;
k) a polypeptide comprising at least about 70% sequence identity to SEQ ID NO: 60;
l) a polypeptide comprising at least about 70% sequence identity to SEQ ID NO: 62, SEQ ID NO: 64 and/or SEQ ID NO: 66; and
m) a polypeptide comprising at least about 70% sequence identity to SEQ ID NO: 68, SEQ ID NO: 70, SEQ ID NO: 72, SEQ ID NO: 74, SEQ ID NO: 76 and/or SEQ ID NO: 78.

The disclosure further relates to a method of diagnosing a subject with arthritis, the method comprising: detecting the presence, absence and/or quantity of one or a plurality of the biomarkers disclosed herein, specifically those identified above. The disclosure also relates to a method of treating a subject with arthritis, the method comprising detecting the presence, absence and/or quantity of one or a plurality of the biomarkers disclosed herein, specifically those identified above, and treating the subject with an arthritis treatment if the presence, absence or quantity of the one or plurality of the biomarkers is at a biologically relevant amount. The disclosure additionally relates to a method identifying prognosis of arthritis in a subject in need thereof, the method comprising detecting the presence, absence and/or quantity of one or a plurality of the biomarkers disclosed herein, specifically those identified above.

In some embodiments, the disclosed methods further comprise obtaining a sample from the subject. In some embodiments, the sample is blood. In some embodiments, the sample is synovium. In some embodiments, the sample is blood and/or synovium.

In some embodiments, the disclosed methods further comprise: ii) calculating a geometric mean expression of up-regulated biomarkers chosen from a) through j) identified above; iii) calculating a geometric mean expression of down-regulated biomarkers chosen from k) through m) identified above; and v) calculating a rheumatoid arthritis score (RAScore) by subtracting the geometric mean expression of the down-regulated biomarkers from the geometric mean expression of the up-regulated biomarkers. In some embodiments, the method further comprises a step of diagnosing the subject as having arthritis if the presence, absence and/or quantity of one or a plurality of the biomarkers disclosed herein are at a biologically significant level or levels. In some embodiments, the biologically relevant amount is at least partially based on the calculated RAScore. In some embodiments, the disclosed methods further comprise a step of diagnosing the subject as having or not having rheumatoid arthritis if the presence, absence and/or quantity of one or a plurality of the biomarkers disclosed herein are at a biologically significant level or levels based at least on the RAScore. In some embodiments, the disclosed methods further comprise comparing the calculated RAScore with a control RAScore calculated from a control dataset obtained from healthy subjects, wherein a higher calculated RAScore is indicative that the subject has arthritis.

Also provided herein is a method of classifying a subject with a subtype of arthritis, the method comprising: i) detecting the presence, absence and/or quantity of one or a plurality of the biomarkers disclosed herein, and ii) calculating a RAScore as described elsewhere herein. In some embodiments, the method further comprises comparing the calculated RAScore with a control RAScore calculated from a control dataset obtained from subjects known to have osteoarthritis, wherein a higher calculated RAScore is indicative of a high likelihood that the subject has rheumatoid arthritis.

Also provided is a method of monitoring the effectiveness of a treatment in a subject having arthritis, the method comprising: i) detecting, before and after treatment, the presence, absence and/or quantity of one or a plurality of the biomarkers disclosed herein, and ii) calculating a pretreatment RAScore and a post-treatment RAScore as described elsewhere herein, wherein a lower post-treatment RAScore as compared to the pre-treatment RAScore is indicative that the treatment is effective.

### BRIEF DESCRIPTION OF DRAWINGS

FIG. 1A-1C depict an overview of the study described in Example 1. FIG. 1A depicts the workflow chart for public data collection, processing and DGE analysis. FIG. 1B depicts the workflow chart for feature selection pipeline. FIG. 1C depicts the workflow chart for gene list validation on the independent datasets. Introducing the RAScore as a geometric mean of validated genes and its association with clinical outcomes.

FIG. 2A-2H show common DE genes between synovium and whole blood tissues. Top Reactome common and different pathways for up-regulated (FIG. 2A) and down-regulated (FIG. 2B) genes. FIG. 2C shows a Venn diagram of up- and down-regulated genes in synovium and blood: 28 common up-regulated genes (p=9e-09) and 4 common down-regulated genes (p=0.28). FIG. 2D shows the comparison scatter plot of fold changes between common genes in synovium and blood. Heatmap and PCA plots of common genes in synovium (FIG. 2E and FIG. 2F) and blood (FIG. 2G and FIG. 211). Vertical bars in the heatmaps represent the color-coded coefficients of variation, Pearson correlations and log 2 fold changes.

FIG. 3A-3F show cell type enrichment analysis for synovium and blood. BH adj p-values<0.05. 30 significant cell types in synovium, 20 significant cell types in WB, 11 common significant cell types.

FIG. 4A-4C depicts feature selected genes. FIG. 4A shows the mean AUC performance of each feature selected gene with standard errors genes on testing synovium and blood data (green) and on five independent validation sets (black). 13 genes with AUC greater than 0.8 for every tissue were chosen as best performing genes. Mean AUC performance with standard errors of a RF model trained on discovery blood data with common DE genes (FIG. 4B) and feature selected genes (FIG. 4C) on five independent validation datasets.

FIG. 5A-5F depicts clinical interpretation of the RAScore. FIG. 5A shows forest plots of correlations of some feature selected genes with DAS28. FIG. 5B shows a forest plot of correlation RAScore with DAS28. FIG. 5C shows RAScore distinguish Healthy, OA and RA samples in synovium. FIG. 5D shows RAScore distinguish Healthy and JIA samples. FIG. 5E shows RAScore tracks the treatment effect in both synovium and blood but shows no difference between RF+ and RF− phenotypes. FIG. 5F shows a forest plot of correlation RAScore with polyarticular Juvenile Idiopathic Arthritis (polyJIA).

FIG. 6A-6H depict PCA plots for synovium and whole blood. FIG. 6A: PCA plot for synovium before batch correction. FIG. 6B: PCA plot for whole blood before batch correction. FIG. 6C: PCA plot for synovium after normalization colored by batch. FIG. 6D: PCA plot for whole blood after normalization colored by batch. FIG. 6E: PCA plot for synovium after normalization colored by treatment type. FIG. 6F: PCA plot for whole blood after normalization colored by treatment type. FIG. 6G: PCA plot for synovium after normalization colored by phenotype. FIG. 611: PCA plot for whole blood after normalization colored by phenotype.

FIG. 7A-7F depict DGE analysis in synovium tissue. FIG. 7A depicts a heatmap and FIG. 7B depicts a PCA plot with DE genes. FIG. 7C depicts up-regulated genes and FIG. 7D depicts the reactome pathways. FIG. 7E depicts down-regulated genes and FIG. 7F depicts the reactome pathways.

FIG. 8A-8F depict DGE analysis in whole blood. FIG. 8A depicts a heatmap and FIG. 8B depicts a PCA plot with DE genes. FIG. 8C depicts up-regulated genes and FIG. 8D depicts the reactome pathways. FIG. 8E depicts down-regulated genes and FIG. 8F depicts the reactome pathways.

FIG. 9 depicts AUROC plots for common and feature selected genes. Three models, a logistic regression, elastic net and random forest, were trained on the discovery whole blood data using either common genes or feature selected genes and validated on 5 validation datasets. The summary curves are the averaged curves with bars of standard errors and colored by red. The dashed and solid lines represent synovium and blood data, respectively.

FIG. 10A-10E depict heatmap and PCA plots of 13 best performing genes on the independent validation. FIG. 10A: synovium RNA-seq GSE89408, FIG. 10B: synovium microarray GSE1919, FIG. 10C: whole blood microarray GSE90081, FIG. 10D: PBMC RNA-seq GSE17755, and FIG. 10E: PBMC microarray GSE15573 datasets.

FIG. 11 depicts correlation forest plots with DAS28 for all 13 feature selected genes.

FIG. 12 depicts correlation of DAS score with RA Score for synovium GSE45867 and blood GSE15258, GSE58795, GSE93272 datasets.

### DETAILED DESCRIPTION OF EMBODIMENTS

Before the present methods and systems are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the methods, devices, and materials in some embodiments are now described. All publications mentioned herein are incorporated by reference in their entirety. Nothing herein is to be construed as an admission that the present disclosure is not entitled to antedate such disclosure by virtue of prior invention.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

An “algorithm,” “formula,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining markers are linear and non-linear equations and statistical classification analyses to determine the relationship between levels of the biomarkers detected in a subject sample and the subject's risk of disease (for example). In panel and combination construction, of particular interest are structural and syntactic statistical classification algorithms, and methods of risk index construction, utilizing pattern recognition features, including established techniques such as cross correlation, Principal Components Analysis (PCA), factor rotation, Logistic Regression (Log Reg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shruken Centroids (SC), StepAIC, Kth-Nearest Neighbor, Boosting, Decision Trees, Neural Networks, Bayesion Networks, Support Vector Machines, and Hidden Markov Models, among others. Many of these techniques are useful either combined with a biomarker selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Leave-One-Out (LOO) and 10-Fold cross-validation (10-Fold-CV).

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

The term “antibody” refers to any immunoglobulin-like molecule that reversibly binds to another with the required selectivity. Thus, the term includes any such molecule that is capable of selectively binding to a biomarker of the present teachings. The term includes an immunoglobulin molecule capable of binding an epitope present on an antigen. The term is intended to encompass not only intact immunoglobulin molecules, such as monoclonal and polyclonal antibodies, but also antibody isotypes, recombinant antibodies, bi-specific antibodies, humanized antibodies, chimeric antibodies, anti-idiopathic (anti-ID) antibodies, single-chain antibodies, Fab fragments, F(ab′) fragments, fusion protein antibody fragments, immunoglobulin fragments, F, fragments, single chain F, fragments, and chimeras comprising an immunoglobulin sequence and any modifications of the foregoing that comprise an antigen recognition site of the required selectivity.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

Ranges provided herein are understood to include all individual integer values and all subranges within the ranges.

“Biomarker,” “biomarkers,” “marker” or “markers” in the context of the present teachings encompasses, without limitation, cytokines, chemokines, growth factors, proteins, peptides, nucleic acids, oligonucleotides, and metabolites, together with their related metabolites, mutations, isoforms, variants, polymorphisms, modifications, fragments, subunits, degradation products, elements, and other analytes or sample-derived measures. Biomarkers can also include mutated proteins, mutated nucleic acids, variations in copy numbers and/or transcript variants. Biomarkers also encompass non-blood borne factors and non-analyte physiological markers of health status, and/or other factors or markers not measured from samples (e.g., biological samples such as bodily fluids), such as clinical parameters and traditional factors for clinical assessments. Biomarkers can also include any indices that are calculated and/or created mathematically. Biomarkers can also include combinations of any one or more of the foregoing measurements, including temporal trends and differences. Biomarkers can include, but are not limited to, TNF alpha induced protein 6 (TNFAIP6), S100 calcium binding protein A8 (S100A8), TNF superfamily member 10 (INFSF/0), DNA damage regulated autophagy modulator 1 (DRAM1, lymphocyte antigen 96 (LY96), glutaminyl-peptide cyclotransferase (QPCT), kynureninase (KYNU), ectonucleoside triphosphate diphosphohydrolase 1 (ENTPDJ), chloride intracellular channel 1 (CLIC1), ATPase H+ transporting VO subunit el (ATP6V0E1), heat shock protein 90 alpha family class B member 1 (HSP90AB1), nucleolin (NCL), and cold inducible RNA binding protein (CIRBP).

The terms “complementary” or “complementarity” refer to polynucleotides (i.e., a sequence of nucleotides) related by base-pairing rules, for example, the sequence “5′-AGT-3′,” is complementary to the sequence “5′-ACT-3′.” Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions. This is of particular importance for methods that depend upon binding between nucleic acid bases.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

“DAS” refers to the Disease Activity Score, a measure of the activity of RA in a subject, well-known to those of skill in the art. See D. van der Heijde et al., Ann. Rheum. Dis. 1990, 49(11):916-920. “DAS” as used herein refers to this particular Disease Activity Score. The “DAS28” involves the evaluation of 28 specific joints. It is a current standard well-recognized in research and clinical practice. Because the DAS28 is a well-recognized standard, it is often simply referred to as “DAS.” Unless otherwise specified, “DAS” herein will encompass the DAS28. A DAS28 can be calculated for an RA subject according to the standard as outlined at the das-score.nl website, maintained by the Department of Rheumatology of the University Medical Centre in Nijmegen, the Netherlands. The number of swollen joints, or swollen joint count out of a total of 28 (SJC28), and tender joints, or tender joint count out of a total of 28 (TJC28) in each subject is assessed. In some DAS28 calculations the subject's general health (GH) is also a factor, and can be measured on a 100 mm Visual Analogue Scale (VAS). GH may also be referred to herein as PG or PGA, for “patient global health assessment” (or merely “patient global assessment”). A “patient global health assessment VAS,” then, is GH measured on a Visual Analogue Scale.

A “dataset,” “set of data” or “data” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence homology to the wild-type sequence or oligo portion of the nucleotide upon which the sequence is derived.

As used herein, the phrase “in need thereof” means that the subject has been identified or suspected as having a need for the particular method or treatment In some embodiments, the identification can be by any means of diagnosis or observation. In any of the methods and treatments described herein, the subject can be in need thereof. In some embodiments, the subject in need thereof is a human seeking treatment for AR. In some embodiments, the subject in need thereof is a human diagnosed with AR. In some embodiments, the subject in need thereof is a human undergoing treatment for AR.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

The term “machine learning method” as used herein encompasses all possible mathematical in silico techniques for creation of useful algorithms from large data sets. The term “algorithm” will be utilized in reference to the clinically useful mathematical equations or computer programs produced by the one or plurality of processes disclosed or executing the the one or plurality of processes disclosed. In some embodiments, the performance of machine learning derived algorithms is independent of the specific in silico software routine used for its derivation. If the same training data set is used, techniques as different as supervised learning, unsupervised learning, association rule learning, hierarchical clustering, multiple linear and logistic regressions are likely to produce algorithms whose clinical performance is indistinguishable.

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any nonhuman mammal. The present disclosure relates to any of the methods or compositions of matter wherein the sample is taken from a mammal or non-human mammal. The present disclosure relates to any of the methods or compositions of matter wherein the sample is taken from a human or non-human primate.

The term “measuring” or “measurement” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters. Alternatively, the term “detecting” or “detection” may be used and is understood to cover all measuring or measurement as described herein.

The term “monitoring” as used herein refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication. In particular, the term “monitoring” can refer to RA staging, RA prognosis, RA inflammation levels, assessing extent of RA progression, monitoring a therapeutic response, predicting a RA score, or distinguishing stable from unstable manifestations of RA disease.

As used herein, the term “normalizing” or “normalized” refers to an expression level of a nucleic acid or protein relative to the mean expression levels of one or a set of reference nucleic acids or proteins. The reference nucleic acids or proteins are based on their minimal variation across tissues or cells.

The particular use of terms “nucleic acid,” “oligonucleotide,” and “polynucleotide” should in no way be considered limiting and may be used interchangeably herein. “Oligonucleotide” is used when the relevant nucleic acid molecules typically comprise less than about 100 bases. “Polynucleotide” is used when the relevant nucleic acid molecules typically comprise more than about 100 bases. Both terms are used to denote DNA, RNA, modified or synthetic DNA or RNA (including, but not limited to nucleic acids comprising synthetic and naturally-occurring base analogs, dideoxy or other sugars, thiols or other non-natural or natural polymer backbones), or other nucleobase containing polymers capable of hybridizing to DNA and/or RNA. Accordingly, the terms should not be construed to define or limit the length of the nucleic acids referred to and used herein, nor should the terms be used to limit the nature of the polymer backbone to which the nucleobases are attached. In some embodiments, the compositions or devices or systems comprise probes specific for binding the biomarkers disclosed herein. In some embodiments, the probes are cDNA or DNA that are complementary to mRNA encoding the biomarkers disclosed herein.

Polynucleotides of the present disclosure may be single-stranded, double-stranded, triple-stranded, or include a combination of these conformations. Generally polynucleotides contain phosphodiester bonds, although in some cases, as outlined below, nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include morpholinos, locked nucleic acids (LNAs), as well as those with positive backbones, non-ionic backbones, and non-ribose backbones. Nucleic acids containing one or more carbocyclic sugars are also included within the definition of nucleic acids. These modifications of the ribose-phosphate backbone may be done to facilitate the addition of additional moieties such as labels, or to increase the stability and half-life of such molecules in physiological environments.

The term “nucleic acid sequence” or “polynucleotide sequence” refers to a contiguous string ofnucleotide bases and in particular contexts also refers to the particular placement ofnucleotide bases in relation to each other as they appear in a polynucleotide.

As used herein in the specification and in the claims, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

As used herein, the term “performance” relates to the quality and overall usefulness of, e.g., a model, algorithm, or prognostic test. Factors to be considered in model or test performance include, but are not limited to, the clinical and analytical accuracy of the test, use characteristics such as stability of reagents and various components, ease of use of the model or test, health or economic value, and relative costs of various reagents and components of the test. Performing can mean the act of carrying out a function. In some embodiments, clinical accuracy

The term “quantitative data” as used herein refers to data associated with any dataset components (e.g., protein markers, clinical indicia, metabolic measures, or genetic assays) that can be assigned a numerical value. Quantitative data can be a measure of the DNA, RNA, or protein level of a marker and expressed in units of measurement such as molar concentration, concentration by weight, etc. For example, if the biomarker is a protein, quantitative data for that biomarker can be protein expression levels measured using methods known to those skill in the art and expressed in mM or mg/dL concentration units.

A “RAScore,” as used herein, is a score that uses quantitative data to provide a quantitative measure of RA disease activity or the state of RA disease in a subject. A set of data from particularly selected biomarkers, such as from the set of biomarkers disclosed herein, is input into an interpretation function according to the present disclosure to derive the RAScore. The interpretation function, in some embodiments, can be created from predictive or multivariate modeling based on statistical algorithms. Input to the interpretation function can comprise the results of testing two or more of the disclosed set of biomarkers, alone or in combination with clinical parameters and/or clinical assessments, also described herein. In some embodiments, the RAScore is a quantitative measure of RA disease activity. As used herein, a RAScore is calculated by subtracting the geometric mean expression of down-regulated biomarkers (e.g., HSP90AB1, NCL, and CIRBP) from the geometric mean expression of up-regulated biomarkers (e.g., TNFAIP6, S100A8, INFSF10, DRAM1, LY96, QPCT, KYNU, ENTPD1, CLIC1, ATP6V0E1).

As used herein, the term “risk” relates to the probability that an event will occur over a specific time period (e.g., developing RA) and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. Alternative continuous measures which may be assessed in the context of the present disclosure include time to health state (e.g., disease) conversion and therapeutic conversion risk reduction ratios.

“Risk evaluation,” or “evaluation of risk” as used herein encompasses making a prediction of the probability, odds, or likelihood that an event or health state may occur, the rate of occurrence of the event or conversion from one health state to another (e.g., from a non-RA condition to a RA condition). Risk evaluation can also comprise prediction of future levels, scores or other indices of disease, either in absolute or relative terms in reference to a previously measured population. The methods of the present disclosure may be used to make continuous or categorical measurements of the risk of conversion between health states. Embodiments of the disclosure can also be used to discriminate between normal and pre-diseased subject cohorts. In other embodiments, the present disclosure may be used so as to discriminate pre-diseased from diseased, or diseased from normal. Such differing use may require different biomarker combinations in individual panel, mathematical algorithm(s), and/or cut-off points, but be subject to the same aforementioned measurements of accuracy for the intended use.

As used herein, the term “sample” refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (C SF), saliva, mucous, sputum, semen, sweat, urine, or any other bodily fluids. “Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art. In some embodiments, the sample is blood. In some embodiments, the sample is synovium or synovial membrane. In some embodiments, samples are taken from a patient or subject that is believed to have RA. In some embodiments, a sample believed to be originated from a patient or subject diagnosed with or suspected of having RA is compared to a “control sample” that is originated from a healthy subject. In some embodiments, a sample believed to be originated from a patient or subject diagnosed with or suspected ofhaving RA is compared to a “control sample” that is originated from a subject known to not having RA. In some embodiments, a sample believed to be originated from a patient or subject diagnosed with or suspected of having RA is compared to a “control sample” that is originated from a subject known to have arthritis other than RA. In some embodiments, a sample believed to be originated from a patient or subject diagnosed with or suspected of having RA is compared to a “control sample” that is originated from a subject known to have osteoarthritis.

A “score” is a value or set of values selected so as to provide a normalized quantitative measure of a variable or characteristic of a subject's condition, and/or to discriminate, differentiate or otherwise characterize a subject's condition. The value(s) comprising the score can be based on, for example, quantitative data resulting in a measured amount of one or more sample constituents obtained from the subject, or from clinical parameters, or from clinical assessments, or any combination thereof. In certain embodiments, the score can be derived from a single constituent, parameter or assessment, while in other embodiments the score is derived from multiple constituents, parameters and/or assessments. The score can be based upon or derived from an interpretation function; e.g., an interpretation function derived from a particular predictive model using any of various statistical algorithms known in the art A “change in score” can refer to the absolute change in score, e.g. from one time point to the next, or the percent change in score, or the change in the score per unit time (i.e., the rate of score change). A “score” as used herein can be used interchangeably with RAScore as defined elsewhere herein. In some embodiments, the score is calculated through an interpretation function or algorithm. In some embodiments, the subject is suspected of having expression of a gene that promotes or contributes to the likelihood of acquiring a disease state or whose expression is correlative to the presence of a pathogen. Calculation of score can be accomplished using known algorithms executable in computer program products within equipment used in sequencing or analyzing samples. In some embodiments, the methods disclosed herein comprise substeps of detecting the presence, absence or quantity of a given biomarker by calculating the quantity of a probe in a control sample, calculating the quantity of a probe in the subject sample, and normalizing the signal obtained from the subject sample by subtracting the signal obtained from the control sample.

As used herein, “sequence identity” is determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). Alternatively, “% sequence identity” can be determined using the EMBOSS Pairwise Alignment Algorithms tool available from The European Bioinformatics Institute (EMBL-EBI), which is part of the European Molecular Biology Laboratory (EMBL). This tool is accessible at the website ebi.ac.uk/Tools/emboss/aligni. This tool utilizes the Needleman-Wunsch global alignment algorithm (Needleman, S. B. and Wunsch, C. D. (1970) J. Mol. Biol. 48, 443-453; Kruskal, J. B. (1983) An overview of sequence comparison, In D. Sankoff and B. Kruskal, (ed.), Time warps, string edits and macromolecules: the theory and practice of sequence comparison, pp. 1-44, Addison Wesley). Default settings are utilized which include Gap Open: 10.0 and Gap Extend 0.5. The default matrix “Blosum62” is utilized for amino acid sequences and the default matrix “DNAfull” is utilized for nucleic acid sequences.

As used herein, the term “statistically significant” means an observed alteration is greater than what would be expected to occur by chance alone (e.g., a “false positive”). Statistical significance can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered highly significant (not random chance) at a p-value less than or equal to 0.05.

As used herein, the term “subject,” “individual” or “patient,” used interchangeably, means any animal, including mammals, such as mice, rats, other rodents, rabbits, dogs, cats, swine, cattle, sheep, horses, or primates, such as humans. A “subject” in the context of the present disclosure is generally a mammal. A subject can be male or female. A subject can be one who has been previously diagnosed or identified as having RA. A subject can be one who has already undergone, or is undergoing, a therapeutic intervention for RA. A subject can also be one who has not been previously diagnosed as having RA; e.g., a subject can be one who exhibits one or more symptoms or risk factors for RA, or a subject who does not exhibit symptoms or risk factors for RA, or a subject who is asymptomatic for RA.

As used herein, the terms “includes,” “including,” “includes,” “including,” “contains,” “containing,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter.

As used herein, the term “plurality” refers to a population of two or more members, such as polynucleotide members or other referenced molecules. In some embodiments, the two or more members of a plurality of members are the same members. For example, a plurality of polynucleotides can include two or more polynucleotide members having the same nucleic acid sequence. In some embodiments, the two or more members of a plurality of members are different members. For example, a plurality of polynucleotides can include two or more polynucleotide members having different nucleic acid sequences. A plurality includes 2, 3, 4, 5, 6, 7, 8, 9, 10, 20, 30, 40, 50, 60, 70, 80, 90 or a 100 or more different members. A plurality can also include 200, 300, 400, 500, 1000, 5000, 10000, 50000, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×106, 9×10⁶ or 1×10⁷ or more different members. A plurality includes all integer numbers in between the above exemplary plurality numbers.

As used herein, the term “target polynucleotide” is intended to mean a polynucleotide that is the object of an analysis or action. The analysis or action includes subjecting the polynucleotide to copying, amplification, sequencing and/or other procedure for nucleic acid interrogation. A target polynucleotide can include nucleotide sequences additional to the target sequence to be analyzed. For example, a target polynucleotide can include one or more adapters, including an adapter that functions as a primer binding site, that flank(s) a target polynucleotide sequence that is to be analyzed. A target polynucleotide hybridized to a capture oligonucleotide or capture primer can contain nucleotides that extend beyond the 5′ or 3′-end of the capture oligonucleotide in such a way that not all of the target polynucleotide is amenable to extension. In particular embodiments, as set forth in further detail below, a plurality of target polynucleotides includes different species that differ in their target polynucleotide sequences but have adapters that are the same for two or more of the different species. The two adapters that can flank a particular target polynucleotide sequence can have the same sequence or the two adapters can have different sequences. Accordingly, a plurality of different target polynucleotides can have the same adapter sequence or two different adapter sequences at each end of the target polynucleotide sequence. Thus, species in a plurality of target polynucleotides can include regions of known sequence that flank regions of unknown sequence that are to be evaluated by, for example, sequencing. In cases where the target polynucleotides carry an adapter at a single end, the adapter can be located at either the 3′-end or the 5′ end the target polynucleotide. Target polynucleotides can be used without any adapter, in which case a primer binding sequence can come directly from a sequence found in the target polynucleotide.

As used herein, the term “capture primers” is intended to mean an oligonucleotide having a nucleotide sequence that is capable of specifically annealing to a single stranded polynucleotide sequence to be analyzed or subjected to a nucleic acid interrogation under conditions encountered in a primer annealing step of, for example, an amplification or sequencing reaction. Generally, the terms “nucleic acid,” “polynucleotide” and “oligonucleotide” are used interchangeably herein. The different terms are not intended to denote any particular difference in size, sequence, or other property unless specifically indicated otherwise. For clarity of description the terms can be used to distinguish one species of nucleic acid from another when describing a particular method or composition that includes several nucleic acid species.

As used herein, the term “target specific” when used in reference to a capture primer or other oligonucleotide is intended to mean a capture primer or other oligonucleotide that includes a nucleotide sequence specific to a target polynucleotide sequence, namely a sequence of nucleotides capable of selectively annealing to an identifying region of a target polynucleotide. Target specific capture primers can have a single species of oligonucleotide, or it can include two or more species with different sequences. Thus, the target specific capture primers can be two or more sequences, including 3, 4, 5, 6, 7, 8, 9 or 10 or more different sequences. The target specific capture oligonucleotides can include a target specific capture primer sequence and universal capture primer sequence. Other sequences such as sequencing primer sequences and the like also can be included in a target specific capture primer.

In comparison, the term “universal” when used in reference to a capture primer or other oligonucleotide sequence is intended to mean a capture primer or other oligonucleotide having a common nucleotide sequence among a plurality of capture primers. A common sequence can be, for example, a sequence complementary to the same adapter sequence. Universal capture primers are applicable for interrogating a plurality of different polynucleotides without necessarily distinguishing the different species whereas target specific capture primers are applicable for distinguishing the different species.

As used herein, the term “immobilized” when used in reference to a nucleic acid is intended to mean direct or indirect attachment to a solid support via covalent or non-covalent bond(s). In certain embodiments of the invention, covalent attachment can be used, but generally all that is required is that the nucleic acids remain stationary or attached to a support under conditions in which it is intended to use the support, for example, in applications requiring nucleic acid amplification and/or sequencing. Typically, oligonucleotides to be used as capture primers or amplification primers are immobilized such that a 3′-end is available for enzymatic extension and at least a portion of the sequence is capable of hybridizing to a complementary sequence. Immobilization can occur via hybridization to a surface attached oligonucleotide, in which case the immobilised oligonucleotide or polynucleotide can be in the 3′-5′ orientation. Alternatively, immobilization can occur by means other than base-pairing hybridization, such as the covalent attachment set forth above.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of rheumatoid arthritis or osteoarthritis. The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue.

A “therapeutic regimen,” “therapy” or “treatment(s),” as described herein, includes all clinical management of a subject and interventions, whether biological, chemical, physical, or a combination thereof, intended to sustain, ameliorate, improve, or otherwise alter the condition of a subject. These terms may be used synonymously herein. Treatments include but are not limited to administration of prophylactics or therapeutic compounds (including conventional DMARDs, biologic DMARDs, non-steroidal anti-inflammatory drugs (NSAID's) such as COX-2 selective inhibitors, and corticosteroids), exercise regimens, physical therapy, dietary modification and/or supplementation, bariatric surgical intervention, administration of pharmaceuticals and/or anti-inflammatories (prescription or over-the-counter), and any other treatments known in the art as efficacious in preventing, delaying the onset of, or ameliorating disease. A “response to treatment” includes a subject's response to any of the above-described treatments, whether biological, chemical, physical, or a combination of the foregoing. A “treatment course” relates to the dosage, duration, extent, etc. of a particular treatment or therapeutic regimen.

Selection of Biomarkers

In some embodiments, the present disclosure relates to a method of selecting a biomarker associated with a disorder or disease. The disclosed methods comprises: a) creating a test data set and a training data set from an input set of data, wherein the input set of data comprises gene expression profiles of subjects having the disorder or disease and control subjects; b) identifying one or a plurality of significant expression profiles correlated with the disorder or disease in the training data set using a statistical test; c) evaluating expression performance of each of the significant expression profiles by applying one or a plurality of machine learning methods to create a performance algorithm; d) testing the performance algorithm on the test data set; e) selecting a high performing expression profile corresponding to at least one biomarker based upon a first threshold of the performance algorithm; f) testing the high performing expression profile selected in step e) with a dataset, said dataset being independent from the input set of data; and g) selecting a biomarker associated with the disorder or disease based on a second threshold of the performance algorithm.

Depending on the target disorder or disease for which selection of biomarkers is undertaken, the input set of data can vary. However, regardless of the target disorder or disease, the input set of data should include dataset from subjects known of having the target disorder or disease as well as dataset from control subjects known of not having the target disorder or disease. As illustrated in Example 1, for instance, publicly available microarray gene expression data at NCBI Gene Expression Omnibus database for whole blood and synovial tissues from RA patients and healthy controls are used. However, the context of microarray gene expression data from RA patients and healthy controls is merely provided for exemplary purposes and is not meant to limit the scope of the disclosed method. For example, if the target disorder or disease is prostate cancer, the input set of data may be publicly available proteomic data or microarray gene expression data from patients known of having prostate cancer and healthy controls. In some embodiments, the target disorder or disease for the disclosed method is arthritis. In some embodiments, the target disorder or disease for the disclosed method is rheumatoid arthritis.

The type of data encompassed in the input set of data can vary as well. In some embodiments, the input set of data comprises microarray gene expression data. In some embodiments, the input set of data comprises proteomic data. In some embodiments, the input set of data comprises RNA-seq data. In some embodiments, the data encompassed in the input set of data is normalized using techniques, including but not limited to, quantile normalization. In some embodiments therefore, the input set of data comprises normalized microarray gene expression data. In some embodiments, the input set of data comprises normalized proteomic data. In some embodiments, the input set of data comprises normalized RNA-seq data.

The data encompassed in the input set of data can be from a single tissue type or a combination of at least two different tissue types. In some embodiments, the input set of data comprises a single tissue type. In some embodiments, the input set of data comprises about two different tissue types. In some embodiments, the input set of data comprises about three different tissue types. In some embodiments, the input set of data comprises about four different tissue types. In some embodiments, the input set of data comprises about five different tissue types. In some embodiments, the input set of data comprises more than about five different tissue types.

Selection of tissue type or tissue types depends on the target disorder or disease. Where the target disorder or disease is RA, as exemplified herein, the tissue type can be blood or synovium. In some embodiments, the input set of data comprises blood data. In some embodiments, the input set of data comprises synovium data. In some embodiments, the input set of data comprises blood data and synovium data.

Once collected, the data can be preprocessed for quality control. For instance, the collected data can be filtered to remove the ones obtained with low number of probes or the ones with poor annotations or duplications. The collected data can also be preprocessed for background correction, probe-gene mapping, treatment annotation, and/or sex annotation and imputation. The preprocessed data can then be merged and normalized across studies using, for instance, Combat for each tissue. The merged data can be further processed for differential gene expression (DGE) analysis, functional analysis, and/or cell type enrichment analysis. In some embodiments therefore, the disclosed method further comprises compiling data from a provider prior to performing step a). In some embodiments, the disclosed method further comprises assessing quality control prior to performing step a). In some embodiments, the disclosed method further comprises data processing normalizing prior to performing step a). In some embodiments, the disclosed method further comprises compiling data from a provider and assessing quality control prior to performing step a). In some embodiments, the disclosed method further comprises compiling data from a provider and data processing normalizing prior to performing step a). In some embodiments, the disclosed method further comprises assessing quality control and data processing normalizing prior to performing step a). In some embodiments, the disclosed method further comprises compiling data from a provider, assessing quality control and data processing normalizing prior to performing step a).

It may occur from time to time that the datasets collected contain expression profile of the same gene, locus or nucleic acid sequence are inconsistent. For example, one dataset may have gene X as up-regulated in patient having RA, but also up-regulated in healthy control in another dataset. Thus, in some embodiments, the disclosed method further comprises eliminating an expression profile of a particular gene, locus or nucleic acid sequence from being a biomarker if the expression profile performance of such a particular gene, locus or nucleic acid sequence is inconsistent between different datasets.

Likewise, it may also occur that expression profile of the same gene, locus or nucleic acid sequence are inconsistent among tissue types. For example, gene X may be up-regulated in a dataset collected from blood of patient having RA, but down-regulated in another dataset collected from synovium of patient having RA. Thus, in some embodiments, the disclosed method further comprises eliminating an expression profile of a particular gene, locus or nucleic acid sequence from being a biomarker if the expression profile performance of such a particular gene, locus or nucleic acid sequence is inconsistent between different tissue types.

To practice the disclosed method, the input set of data is stratified sampled into a test data set and a training data set. The training data set is used to create a performance algorithm, while the test data set is used for the validation of the performance algorithm. In some embodiments, the test data set and the training data set comprise a random spilt of the input set of data in a ratio of about 1:2. In some embodiments, the test data set and the training data set comprise a random spilt of the input set of data in a ratio of about 1:3. In some embodiments, the test data set and the training data set comprise a random spilt of the input set of data in a ratio of about 1:4. In some embodiments, the test data set and the training data set comprise a random spilt of the input set of data in a ratio of about 1:5. In some embodiments, the test data set and the training data set comprise a random spilt of the input set of data in a ratio of about 1:6. In some embodiments, the test data set and the training data set comprise a random spilt of the input set of data in a ratio of about 1:7. In some embodiments, the test data set and the training data set comprise a random spilt of the input set of data in a ratio of about 1:8. In some embodiments, the test data set and the training data set comprise a random spilt of the input set of data in a ratio of about 1:9. In some embodiments, the test data set and the training data set comprise a random spilt of the input set of data in a ratio of about 1:10.

To create a performance algorithm, one or a plurality of significant expression profiles correlated with the target disorder or disease are identified in the training data set using a statistical test. The selection of a significant expression profile correlated with the target disorder or disease is based on estimating the false discovery rate (FDR) through the q-values. This step includes using several tests aimed at finding the values where the average or the variance of the expression signals or intensities in different phenotypes are significantly different. The following tests may be applied.

The t-test may be used, which uses the t-statistics t=(μ1−μ2)/(σ1 2/n1+σ2 2/n2)½ to determine if the means μ1 and μ2 of the expression signals or intensities of an expression profile across the samples in the two different profiles are different; σ1 and σ2 are the corresponding standard deviation of the intensity levels, and n1, n2 are the number of samples in the two profiles.

The signal-to-noise ratio, which is a variant of the t-statistic, defined as s2n=(μ1−μ2)/(σ1+σ2), may also be applied.

The Pearson correlation coefficient, which is the correlation between the expression signals or intensities of an expression profile across the samples and the phenotype vector of the samples, may also be used.

The F-test, may also be used and is based on the ratio of the average square deviations from the mean between the two phenotypes (F statistics), and determines if the standard deviations of the expression signals or intensities of an expression profile across the samples are different in the two phenotypes. Each of these tests assigned a p-value to each peptide, which are determined by permutation.

In embodiments where the datasets comprise microarray data and/or RNA-seq data, the package “limma” (stand for linear models for microarray data), a package for the analysis of gene expression data arising from microarray or RNA-seq (Ritchie, M. E., Phipson, B., Wu, D., Hu, Y., Law, C. W., Shi, W., and Smyth, G. K. (2015). Limma powers differential expression analyses for RNA-sequencing and microarray studies. Nucleic Acids Research 43, e47) can be used. In some embodiments, a significant expression profile is identified using limma with an FDR p-value<0.05. In some embodiments, a Pearson correlation can be computed for each significant expression profile identified with the case-control status, and those with r<0.25 can be filtered out. In some embodiments, gene pair-wise correlations can be computed and expression profiles with correlation greater than 0.8 can be removed for robustness and reducing gene redundancy.

The significant expression profiles identified are then subjected to multiple evaluations, which involves applying several machine learning methods to the training data to create a performance algorithm for the test data set. Specifically, the data are trained using one or a combination of machine learning methods, including but not limited to, linear regression, logistic regression, elastic net, decision tree, and random forest.

Linear regression is an approach for predicting a quantitative response Y on the basis of a single predictor variable X, assuming a linear relationship between X and Y. The following formula is generally used for this machine learning method.

Y=β₀+β₁X

Logistic regression models the probability that Y belongs to a particular binary category using logit transformation that is linear in X. The following formula is generally used for this machine learning method.

$$p ( X ) = Pr ( Y = 1 \vert X ) = \frac{{e}^{{β}_{0} + {β}_{1} X}}{1 + {e}^{{β}_{0} + {β}_{1} X}}$$

Elastic net is a regularized regression method that linearly combines the L1 and L2 penalties of the lasso and ridge methods. The following formula is generally used to calculate the elastic net penalty.

J(β)=α∥β∥²+(1−α)∥β∥₁

Decision tree is a decision support tool that uses a tree-like graph or model of decisions and their possible consequences, including chance event outcomes, resource costs, and utility. To create a decision tree, the following steps are generally used:
1. Use recursive binary splitting to grow a large tree on the training data, stopping only when each terminal node has fewer than some minimum number of observations;
2. Apply cost complexity pruning to the large tree in order to obtain a sequence of best subtrees, as a function of a;
3. Use K-fold cross-validation to choose a. That is, divide the training observations into K folds. For each k=1, . . . , K:
   a. Repeat Steps 1 and 2 on all but the kth fold of the training data; and
   b. Evaluate the classification error rate, or Gini index, or entropy on the data in the left-out kth fold, as a function of α.
   Average the results for each value of α, and pick α to minimize the average error; and
4. Return the subtree from Step 2 that corresponds to the chosen value of α.

Random forest, or random decision forest, is an ensemble learning method for classification, regression and other tasks that operate by constructing a multitude of decision trees at training time and outputting the class that is the mode of the classes (classification) or mean prediction (regression) of the individual trees. To create a random forest, the following steps are generally used:
1. For b=1 to B:
   a. Draw a bootstrap sample Z* of size N from the training data;
   b. Grow a random-forest tree T_{b} to the bootstrapped data, by re-cursively repeating the following steps for each terminal node of the tree, until the minimum node size nₘᵢₙ is reached:
      i. Select m variables at random from the p variables;
      ii. Pick the best variable/split-point among the m; and
      iii. Split the node into two daughter nodes;
2. Output the ensemble of trees {T_{b}}₁^{B}.
   To make a prediction at a new point x: let Ĉ_{b}(x) be the class prediction of the bth random-forest tree. Then, Ĉ_{rf}^{B}(x)=majority vote {Ĉ_{b}(x)}₁^{B}.

In some embodiments, the machine learning method used in step c) of the disclosed method comprise one or a combination of linear regression, logistic regression, decision tree, elastic net and random forest. In some embodiments, the machine learning method used in step c) of the disclosed method comprises linear regression. In some embodiments, the machine learning method used in step c) of the disclosed method comprises logistic regression. In some embodiments, the machine learning method used in step c) of the disclosed method comprises decision tree. In some embodiments, the machine learning method used in step c) of the disclosed method comprises elastic net. In some embodiments, the machine learning method used in step c) of the disclosed method comprises random forest.

Once a performance algorithm is created, it is then tested on the test data set for accuracy. This validation can be performed using any methods known in the art, such as area under receiver operating characteristic curve (AUROC). In some embodiments, the performance algorithm created by the disclosed method is validated in the test data set using AUROC.

In some embodiments, the steps a) through d) described above can be repeated several times. Repeating those steps can be important to minimize bias of a random split of the input set of data into training and testing sets. In some embodiments, the steps a) through d) are repeated from at least about 2 to about 100 times. In some embodiments, the steps a) through d) are repeated from at least about 5 to about 150 times. In some embodiments, the steps a) through d) are repeated from at least about 10 to about 200 times. In some embodiments, the steps a) through d) are repeated from at least about 20 to about 80 times. In some embodiments, the steps a) through d) are repeated from at least about 30 to about 60 times. In some embodiments, the steps a) through d) are repeated for about 10 times. In some embodiments, the steps a) through d) are repeated for about 20 times. In some embodiments, the steps a) through d) are repeated for about 30 times. In some embodiments, the steps a) through d) are repeated for about 40 times. In some embodiments, the steps a) through d) are repeated for about 50 times. In some embodiments, the steps a) through d) are repeated for about 60 times. In some embodiments, the steps a) through d) are repeated for about 70 times. In some embodiments, the steps a) through d) are repeated for about 80 times. In some embodiments, the steps a) through d) are repeated for about 90 times. In some embodiments, the steps a) through d) are repeated for about 100 times. In some embodiments, the steps a) through d) are repeated for about 110 times. In some embodiments, the steps a) through d) are repeated for about 120 times. In some embodiments, the steps a) through d) are repeated for more than about 120 times.

Once a performance algorithm is created and validated by testing with the test data set, it can be used to select a high performing expression profile corresponding to at least one biomarker associated with the target disorder or disease based upon a first threshold of the performance algorithm. In the case when the performance algorithm is validated with AUROC, the first threshold for selecting a high performing expression profile can be a cutoff line of a selected mean AUROC. As would be understood by one skilled in the art, the higher this first threshold is, the less potential biomarkers will be identified. Thus, it is important to choose an appropriate threshold that is not too high and not too low as well. In some embodiments, the first threshold for selecting a high performing expression profile in the disclosed method is a mean AUROC from about 0.5 to about 0.9. In some embodiments, the first threshold for selecting a high performing expression profile is a mean AUROC from about 0.6 to about 0.8. In some embodiments, the first threshold for selecting a high performing expression profile is a mean AUROC of about 0.5. In some embodiments, the first threshold for selecting a high performing expression profile is a mean AUROC of about 0.6. In some embodiments, the first threshold for selecting a high performing expression profile is a mean AUROC of about 0.67 (or ⅔). In some embodiments, the first threshold for selecting a high performing expression profile is a mean AUROC of about 0.7. In some embodiments, the first threshold for selecting a high performing expression profile is a mean AUROC of about 0.8. In some embodiments, the first threshold for selecting a high performing expression profile is a mean AUROC of about 0.9.

The high performing expression profiles selected in step e) as described above are further validated and tested with one or a plurality of datasets that are independent from the input set of data initially used. In the case when the performance algorithm is validated with AUROC, this further validation and testing of the high performing expression profiles can also be performed with AUROC. Once validated, biomarkers associated with the target disorder or disease can be then selected based upon a second threshold of the performance algorithm. In the case when the first threshold for selecting a high performing expression profile is a selected mean AUROC, this second threshold for selecting biomarkers associated with the target disorder or disease can also be a mean AUROC that is higher than the first threshold. In some embodiments, the second threshold is a mean AUROC from about 0.6 to about 0.9. In some embodiments, the second threshold is a mean AUROC from about 0.7 to about 0.9. In some embodiments, the second threshold is a mean AUROC from about 0.8 to about 0.9. In some embodiments, the second threshold is a mean AUROC equal to or higher than about 0.6. In some embodiments, the second threshold is a mean AUROC equal to or higher than about 0.7. In some embodiments, the second threshold is a mean AUROC equal to or higher than about 0.8. In some embodiments, the second threshold is a mean AUROC equal to or higher than about 0.9.

It is contemplated by the disclosure that any biomarker selected following the disclosed method is also encompassed by the present disclosure.

Biomarkers for RA

The disclosure further relates to biomarkers for RA and their applications thereof. Using datasets obtained from publicly available microarray gene expression data at NCBI Gene Expression Omnibus database for whole blood and synovial tissues from RA patients and healthy controls, a set of biomarkers consisting of 13 genes is obtained. A summary of this set of 13 biomarkers is provided in Table A.

| Gene Symbol | Gene Name | Reactome Pathways |
|---|---|---|
| TNFAIP6 | TNF alpha induced | Innate Immune System, Neutrophil degranulation, Immune System |
| | protein 6 | |
| S100A8 | S100 calcium | Signal Transduction, Innate Immune System, Toll-like Receptor Cascades, |
| | binding protein A8 | Neutrophil degranulation, Immune System, Antimicrobial peptides, RHO |
| | | GTPase Effectors, Regulation of TLR by endogenous ligand, RHO GTPases |
| | | Activate NADPH Oxidases, Signaling by Rho GTPases, Metal sequestration |
| | | by antimicrobial proteins |
| DRAM1 | DNA damage | — |
| | regulated autophagy | |
| | modulator 1 | |
| TNFSF10 | Tumor necrosis | Death Receptor Signalling, Regulation by c-FLIP, Regulation of necroptotic |
| | factor superfamily | cell death, RIPK1-mediated regulated necrosis, TRAIL signaling, Signal |
| | member 10 | Transduction, CASP8 activity is inhibited, Regulated Necrosis, Apoptosis, |
| | | Caspase activation via extrinsic apoptotic signalling pathway, Programmed |
| | | Cell Death, Dimerization of procaspase-8, Caspase activation via Death |
| | | Receptors in the presence of ligand |
| LY96 | Lymphocyte antigen | Toll Like Receptor 2 (TLR2) Cascade, IRAK4 deficiency (TLR2/4), |
| | 96 | TRAF6-mediated induction of TAK1 complex within TLR4 complex, |
| | | TRIF-mediated programmed cell death, MyD88 deficiency (TLR2/4), Toll |
| | | Like Receptor 7/8 (TLR7/8) Cascade, Activation of IRF3/IRF7 mediated by |
| | | TBK1/IKK epsilon, Innate Immune System, IRAK2 mediated activation of |
| | | TAK1 complex upon TLR7/8 or 9 stimulation, MyD88-independent TLR4 |
| | | cascade, Apoptosis, etc. |
| QPCT | Glutaminyl-peptide | Innate Immune System, Neutrophil degranulation, Immune System |
| | cyclotransferase | |
| KYNU | Kynureninase | Metabolism, Metabolism of amino acids and derivatives, Tryptophan |
| | | catabolism |
| ENTPD1 | Ectonucleoside | Metabolism, Metabolism of nucleotides, Nucleobase catabolismo Phosphate |
| | triphosphate | bond hydrolysis by NTPDase proteins |
| | diphosphohydrolase 1 | |
| CLIC1 | Chloride | — |
| | intracellular channel 1 | |
| ATP6V0E1 | ATPase H+ | Cellular responses to stress, Amino acids regulate mTORC1, ROS and RNS |
| | transporting V0 | production in phagocytes, Cellular responses to external stimuli, Insulin |
| | subunit e1 | receptor recycling, Transferrin endocytosis and recycling, Signaling by |
| | | Insulin receptor, Signal Transduction, Innate Immune System, Immune |
| | | System, Iron uptake and transport, Signaling by Receptor Tyrosine Kinases, |
| | | Transport of small molecules, Ion channel transport |
| NCL | Nucleolin | Major pathway of rRNA processing in the nucleolus and cytosol, rRNA |
| | | processing in the nucleus and cytosol, Metabolism of RNA, rRNA |
| | | processing |
| CIRBP | Cold inducible RNA | — |
| | binding protein | |
| HSP90AB1 | Heat shock protein | Cell Cycle, Mitotic, Inflammasomes, Cellular responses to stress, G2/M |
| | 90 alpha family class | Transition, Attenuation phase, Cellular responses to external stimuli, ESR- |
| | B member 1 | mediated signaling, Sema3A PAK dependent Axon repulsion, Infectious |
| | | disease, Biological oxidations, Signal Transduction, Innate Immune System, |
| | | Fcgamma receptor (FCGR) dependent phagocytosis, Chaperone Mediated |
| | | Autophagy, etc. |

Among these 13 biomarkers, TNFAIP6, S100A8, DRAM1, TNFSF 10, LY96, QPCT, KYNU, ENTPD1, CLIC1 and ATP6V0E1 are up-regulated in RA patients, while NCL, CIRBP and HSP90AB1 are down-regulated in RA patients. Representative nucleic acid sequences and protein sequences for these 13 biomarker genes are provided in Table B.

| | Gene | mRNA/CDNA | | Protein | |
|---|---|---|---|---|---|
| Gene | name | RefSeq ID | mRNA/cDNA Sequence | RefSeq ID | Protein Sequence |
| TNFAIP6 | TNF | NM_007115.4 | AGTCACATTTCAGCCACTGCTCTG | NP_009046.2 | MILIYLFLLLWEDTQG |
| | Alpha | | AGAATTTGTGAGCAGCCCCTAACA | | WGFKDGIFHNSIWLERA |
| | Induced | | GGCTGTTACTTCACTACAACTG | | AGVYHREARS |
| | Protein 6 | | ACGATATGATCATCTTAATTTACTT | | GKYKLTYAEAKAVCEF |
| | | | ATTTCTCTTGCTATGGGAAGACACT | | EGCHLATYKQLEAARKI |
| | | | CAAGGATGGGGATTCAAGGA | | GFHVCAAGWMAKGRV |
| | | | TGGAATTTTTCATAACTCCATATGG | | GYPIVKPGPN |
| | | | CTTGAACGAGCAGCCGGTGTGTAC | | CGFGKTGHIDYGIRLNRS |
| | | | CACAGAGAAGCACGGTCTGGC | | ERWDAYCYNPHAKECG |
| | | | AAATACAAGCTCACCTACGCAGAA | | GVFTDPKQIFKSPGFPNE |
| | | | GCTAAGGCGGTGTGTGAATTTGAA | | YEDNQI |
| | | | GGCGGCCATCTCGCAACTTACA | | CYWHIRLKYGQRIHLSF |
| | | | AGCAGCTAGAGGCAGCCAGAAAA | | LDFDLEDDPGCLADYV |
| | | | ATTGGATTTCATGTCTGTGCTGCTG | | EIYDSYDDVHCFVGRY |
| | | | GATGGATGGCTAAGGGCAGAGT | | CGDELPDDI |
| | | | TGGATACCCCATTGTGAAGCCAGG | | ISTGNVMTLKFLSDASV |
| | | | GCCCAACTGTGGATTTGGAAAAAC | | TAGGFQIKYVAMDPVS |
| | | | TGGCATTATTGATTATGGAATC | | KSSQGKNTSTTSTGNKN |
| | | | CGTCTCAATAGGAGTGAAAGATGG | | FLAGRFSHL (SEQ ID |
| | | | GATGCCTATTGCTACAACCCACAC | | NO: 2) |
| | | | GCAAAGGAGTGTGGTGGCGTCT | | |
| | | | TTACAGATCCAAAGCAAATTTTTA | | |
| | | | AATCTCCAGGCTTCCCAAATGAGT | | |
| | | | ACGAAGATAACCAAATCTGCTA | | |
| | | | CTGGCACATTAGACTCAAGTATGG | | |
| | | | TCAGCGTATTCACCTGAGTTTTTTA | | |
| | | | GATTTTGACCTTGAAGATGAC | | |
| | | | CCAGGTTGCTTGGCTGATTATGTTG | | |
| | | | AAATATATGACAGTTACGATGATG | | |
| | | | TCCATGGCTTTGTGGGAAGAT | | |
| | | | ACTGTGGAGATGAGCTTCCAGATG | | |
| | | | ACATCATCAGTACAGGAAATGTCA | | |
| | | | TGACCTTGAAGTTTCTAAGTGA | | |
| | | | TGCTTCAGTGACAGCTGGAGGTTT | | |
| | | | CCAAATCAAATATGTTGCAATGGA | | |
| | | | TCCTGTATCCAAATCCAGTCAA | | |
| | | | CGAAAAAATACAAGTACTACTTCT | | |
| | | | ACTGGAAATAAAAACTTTTTAGCT | | |
| | | | GGAAGATTTAGCCACTTATAAA | | |
| | | | AAAAAAAAAAAGGATGATCAAAA | | |
| | | | CACACAGTGTTTATGTTGGAATCTT | | |
| | | | TTGGAACTCCTTTGATCTCACT | | |
| | | | GTTATTATTAACATTTATTTATTAT | | |
| | | | TTTTCTAAATGTGAAAGCAATACA | | |
| | | | TAATTTAGGGAAAATTCGAAA | | |
| | | | ATATAGGAAACTTTAAACGAGAAA | | |
| | | | ATGAAACCTCTCATAATCCCACTG | | |
| | | | CATAGAAATAACAAGCGTTAAC | | |
| | | | ATTTTCATATTTTTTTCTTTCAGTCA | | |
| | | | TTTTTCTATTTGTGGTATATGTATA | | |
| | | | TATGTACCTATATGTATTT | | |
| | | | GCATTTGAAATTTTGGAATCCTGCT | | |
| | | | CTATGTACAGTTTTGTATTATACTT | | |
| | | | TTTAAATCTTGAACTTTATA | | |
| | | | AACATTTTCTGAAATCATTGATTAT | | |
| | | | TCTACAAAAACATGATTTTAAACA | | |
| | | | GCTGTAAAATATTCTATGATA | | |
| | | | TGAATGTTTTATGCATTATTTAAGC | | |
| | | | CTGTCTCTATTGTTGGAATTTCAGG | | |
| | | | TCATTTTCATAAATATTGTT | | |
| | | | GCAATAAATATCCTTGAACACA | | |
| | | | (SEQ ID NO: 1) | | |
| S100A8 | S100 | NM_001319196.1 | GAGAAACCAGAGACTGTAGCAACT | NP_001306125.1 | MSLVSCLSEDLKVLFFR |
| | Calcium | | CTGGCAGGGAGAAGCTGTCTCTGA | | WGKSVGIMLTELEKALN |
| | Binding | | TGGCCTGAAGCTGTGGGCAGCT | | SIIDVYHKYS |
| | Protein | | GGCCAAGCCTAACCGCTATAAAAA | | LIKGNFHAVYRDDLKKL |
| | A8 | | GGAGCTGCCTCTCAGCCCTGCATG | | LETECPQYIRKKGADVW |
| | | | TCTCTTGTCAGCTGTCTTTCAG | | FKELDINTDGAVNFQEF |
| | | | AAGACCTGAAGGTTCTGTTTTTCA | | LILVIKM |
| | | | GGTGGGGCAAGTCCGTGGGCATCA | | GVAAHKKSHEESHKE |
| | | | TGTTGACCGAGCTGGAGAAAGC | | (SEQ ID NO: 4) |
| | | | CTTGAACTCTATCATCGACGTCTAC | | |
| | | | CACAAGTACTCCCTGATAAAGGGG | | |
| | | | AATTTCCATGCCGTCTACAGG | | |
| | | | GATGACCTGAAGAAATTGCTAGAG | | |
| | | | ACCGAGTGTCCTCAGTATATCAGG | | |
| | | | AAAAAGGGTGCAGACGTCTGGT | | |
| | | | TCAAAGAGTTGGATATCAACACTG | | |
| | | | ATGGTGCAGTTAACTTCCAGGAGT | | |
| | | | TCCTCATTCTGGTGATAAAGAT | | |
| | | | GGGCGTGGCAGCCCACAAAAAAA | | |
| | | | GCCATGAAGAAAGCCACAAAGAG | | |
| | | | TAGCTGAGTTACTGGGCCCAGAGG | | |
| | | | CTGGGCCCCTGGACATGTACCTGC | | |
| | | | AGAATAATAAAGTCATCAATACCT | | |
| | | | CAAAAAAAAAA (SEQ ID NO: 3) | | |
| | | NM_001319197.1 | GAGAAACCAGAGACTGTAGCAACT | NP_001306126.1 | MSLVSCLSEDLVLFFRW |
| | | | CTGGCAGGGAGAAGCTGTCTCTGA | | GKSVGIMLTELEKALNSI |
| | | | TGGCCTGAAGCTGTGGGCAGCT | | IDVYHKYSL |
| | | | GGCCAAGCCTAACCGCTATAAAAA | | IKGNFHAVYRDDLKKLL |
| | | | GGAGCTGCCTCTCAGCCCTGCATG | | ETECPQYIRKKGADVWF |
| | | | TCTCTTGTCAGCTGTCTTTCAG | | KELDINTDGAVNFQEFLI |
| | | | AAGACCTGGTTCTGTTTTTCAGGTG | | LVIKMG |
| | | | GGGCAAGTCCGTGGGCATCATGTT | | VAAHKKSHEESHKE |
| | | | GACCGAGCTGGAGAAAGCCTT | | (SEQ ID NO: 6) |
| | | | GAACTCTATCATCGACGTCTACCA | | |
| | | | CAAGTACTCCCTGATAAAGGGGAA | | |
| | | | TTTCCATGCCGTCTACAGGGAT | | |
| | | | GACCTGAAGAAATTGCTAGAGACC | | |
| | | | GAGTGTCCTCAGTATATCAGGAAA | | |
| | | | AAGGGTGCAGACGTCTGGTTCA | | |
| | | | AAGAGTTGGATATCAACACTGATG | | |
| | | | GTGCAGTTAACTTCCAGGAGTTCC | | |
| | | | TCATTCTGGTGATAAAGATGGG | | |
| | | | CGTGGCAGCCCACAAAAAAAGCC | | |
| | | | ATGAAGAAAGCCACAAAGAGTAG | | |
| | | | CTGAGTTACTGGGCCCAGAGGCTG | | |
| | | | GGCCCCTGGACATGTACCTGCAGA | | |
| | | | ATAATAAAGTCATCAATACCTCAA | | |
| | | | AAAAAAAA (SEQ ID NO: 5) | | |
| | | NM_001319198.1 | TGTTTTGATATCAGAATTTCTGGGG | NP_001306127.1 | MWGKSVGIMLTELEKA |
| | | | AACATTTGGATTTCCAGAATCTCTT | | LNSIIDVYHKYSLIKGNF |
| | | | TCACATCAGCTGTAATGTGG | | HAVYRDDLKK |
| | | | GGCAAGTCCGTGGGCATCATGTTG | | LLETECPQYIRKKGADV |
| | | | ACCGAGCTGGAGAAAGCCTTGAAC | | WFKELDINTDGAVNFQE |
| | | | TCTATCATCGACGTCTACCACA | | FLILVIKMGVAAHKKSH |
| | | | AGTACTCCCTGATAAAGGGGAATT | | EESHKE (SEQ ID NO: 8) |
| | | | TCCATGCCGTCTACAGGGATGACC | | |
| | | | TGAAGAAATTGCTAGAGACCGA | | |
| | | | GTGTCCTCAGTATATCAGGAAAAA | | |
| | | | GGGTGCAGACGTCTGGTTCAAAGA | | |
| | | | GTTGGATATCAACACTGATGGT | | |
| | | | GCAGTTAACTTCCAGGAGTTCCTC | | |
| | | | ATTCTGGTGATAAAGATGGGCGTG | | |
| | | | GCAGCCCACAAAAAAAGCCATG | | |
| | | | AAGAAAGCCACAAAGAGTAGCTG | | |
| | | | AGTTACTGGGCCCAGAGGCTGGGC | | |
| | | | CCCTGGACATGTACCTGCAGAAT | | |
| | | | AATAAAGTCATCAATACCTCAAAA | | |
| | | | AAAAAA (SEQ ID NO: 7) | | |
| | | NM_001319201.1 | ATGTCTCTTGTCAGCTGTCTTTCAG | NP_002955.2 | MLTELEKALNSIIDVYH |
| | | | AAGACCTGGTGGGGCAAGTCCGTG | | KYSLIKGNFHAVYRDDL |
| | | | GGCATCATGTTGACCGAGCTG | | KKLLETECPQ |
| | | | GAGAAAGCCTTGAACTCTATCATC | | YIRKKGADVWFKELDIN |
| | | | GACGTCTACCACAAGTACTCCCTG | | TDGAVNFQEFLILVIKM |
| | | | ATAAAGGGGAATTTCCATGCCG | | GVAAHKKSHEESHKE |
| | | | TCTACAGGGATGACCTGAAGAAAT | | (SEQ ID NO: 10) |
| | | | TGCTAGAGACCGAGTGTCCTCAGT | | |
| | | | ATATCAGGAAAAAGGGTGCAGA | | |
| | | | CGTCTGGTTCAAAGAGTTGGATAT | | |
| | | | CAACACTGATGGTGCAGTTAACTT | | |
| | | | CCAGGAGTTCCTCATTCTGGTG | | |
| | | | ATAAAGATGGGCGTGGCAGCCCAC | | |
| | | | AAAAAAAGCCATGAAGAAAGCCA | | |
| | | | CAAAGAGTAGCTGAGTTACTGGG | | |
| | | | CCCAGAGGCTGGGCCCCTGGACAT | | |
| | | | GTACCTGCAGAATAATAAAGTCAT | | |
| | | | CAATACCTCA (SEQ ID NO: 9) | | |
| | | NM_002964.5 | GAGCAGCCTTCCTGAGAGAGGAGA | NP_001306130.1 | MLTELEKALNSIIDVYH |
| | | | GAGAAAGCTCAGGGAGGTCTGGA | | KYSLIKGNFHAVYRDDL |
| | | | GCAAAGATACTCCTGGAGGTGGG | | KKLLETECPQ |
| | | | GAGTGAGGCAGGGATAAGGAAGG | | YIRKKGADVWFKELDIN |
| | | | AGAGTATCCTCCAGCACCTTCCAG | | TDGAVNFQEFLILVIKM |
| | | | TGGGTGGGGCAAGTCCGTGGGCA | | GVAAHKKSHEESHKE |
| | | | TCATGTTGACCGAGCTGGAGAAAG | | (SEQ ID NO: 12) |
| | | | CCTTGAACTCTATCATCGACGTCTA | | |
| | | | CCACAAGTACTCCCTGATAAA | | |
| | | | GGGGAATTTCCATGCCGTCTACAG | | |
| | | | GGATGACCTGAAGAAATTGCTAGA | | |
| | | | GACCGAGTGTCCTCAGTATATC | | |
| | | | AGGAAAAAGGGTGCAGACGTCTG | | |
| | | | GTTCAAAGAGTTGGATATCAACAC | | |
| | | | TGATGGTGCAGTTAACTTCCAGG | | |
| | | | AGTTCCTCATTCTGGTGATAAAGA | | |
| | | | TGGGCGTGGCAGCCCACAAAAAA | | |
| | | | AGCCATGAAGAAAGCCACAAAGA | | |
| | | | GTAGCTGAGTTACTGGGCCCAGAG | | |
| | | | GCTGGGCCCCTGGACATGTACCTG | | |
| | | | CAGAATAATAAAGTCATCAATA | | |
| | | | CCTCAAAAAAAAAA (SEQ ID NO: | | |
| | | | 11) | | |
| DRAM1 | DNA | NM_018370.3 | ACTCTGGCCCGGCAGCCTCGCCGC | NP_060840.2 | MLCFLRGMAFVPFLLV |
| | Damage | | CCGCAGCCTCGCTCCGCTCCTCGC | | TWSSAAFIISYVVAVLS |
| | Regulated | | GCTTCCCCTCCCTCCGGGGCTG | | GHVNPFLPYIS |
| | Autophagy | | GGCCTGCCCCGGCCGTCGCGGAGC | | DIGTTPPESGIFGFMINF |
| | Modulator | | CTCCCCTCCCACCGTCCGTGAGTGT | | SAFLGAATMYTRYKIV |
| | 1 | | ACGCGCCCGGCCGCCGCCTCC | | QKQNQTCYFSTPVFNLV |
| | | | AGGCAGCCCGGAGCAACCCGGCG | | SLVLGLV |
| | | | CCCGGCCCCGCTGGGCGCAGCACT | | GCFGMGIVANFQELAV |
| | | | CCGTCGGCGGCGGCGGCGGCGCG | | PVVHDGGALLAFVCGV |
| | | | ATGCTGTGCTTCCTGAGGGGAATG | | VYTLLQSIISYKSCPQW |
| | | | GCTTTCGTCCCCTTCCTCTTGGTGA | | NSLSTCHIR |
| | | | CCTGGTCGTCAGCCGCCTTCA | | MVISAVSCAAVIPMIVC |
| | | | TTATCTCCTACGTGGTCGCCGTGCT | | ASLISITKLEWNPREKD |
| | | | CTCCGGGCACGTCAACCCCTTCCT | | YVYHVVSAICEWTVAF |
| | | | CCCGTATATCAGTGATACGGG | | GFIFYFLT |
| | | | AACAACACCTCCAGAGAGTGGTAT | | FIQDFQSVTLRISTEING |
| | | | TTTTGGATTTATGATAAACTTCTCT | | DI (SEQ ID NO: 14) |
| | | | GCATTTCTTGGTGCAGCCACG | | |
| | | | ATGTATACAAGATACAAAATAGTA | | |
| | | | CAGAAGCAAAATCAAACCTGCTAT | | |
| | | | TTCAGCACTCCTGTTTTTAACT | | |
| | | | TGGTGTCTTTAGTGCTTGGATTGGT | | |
| | | | GGGATGTTTCGGAATGGGCATTGT | | |
| | | | CGCCAATTTTCACGAGTTAGC | | |
| | | | TGTGCCAGTGGTTCATGACGGGGG | | |
| | | | CGCTCTTTTGGCCTTTGTCTGTGGT | | |
| | | | GTCGTGTACACGCTCCTACAG | | |
| | | | TCCATCATCTCTTACAAATCATGTC | | |
| | | | CCCAGTGGAACAGTCTCTCGACAT | | |
| | | | GCCACATACGGATGGTCATCT | | |
| | | | CTGCCGTTTCTTGCGCAGCTGTCAT | | |
| | | | CCCCATGATTGTCTGTGCTTCACTA | | |
| | | | ATTTCCATAACCAAGCTGGA | | |
| | | | GTGGAATCCAAGAGAAAAGGATTA | | |
| | | | TGTATATCACGTAGTGAGTGCGAT | | |
| | | | CTGTGAATGGACAGTGGCCTTT | | |
| | | | GGTTTTATTTTCTACTTCCTAACTT | | |
| | | | TCATCCAAGATTTCCAGACTGTCA | | |
| | | | CCCTAAGGATATCCACAGAAA | | |
| | | | TCAATGGTGATATTTGAAGAAAGA | | |
| | | | AGAATTCAGTCTCACTCAGTGAAT | | |
| | | | GTCGCAGGCCATTTCTAAAAGT | | |
| | | | GCTACAGAGGACAGACAGGGTTTT | | |
| | | | GAGGCCACCCTGATTATTGGGATG | | |
| | | | CATCTGCAGCACATCCAGGACT | | |
| | | | TGAATTTCATTACGAGTTCCTAATA | | |
| | | | GTTGTATTTCTAAAGATGTGTTTCC | | |
| | | | TAGAGAATGTACAGCCTTAT | | |
| | | | GACACTGTAGTGATGTTTTTATAAT | | |
| | | | TTTCTAAGTAGATTTTTTTATATTA | | |
| | | | ACAAATTCATATACACAAAA | | |
| | | | AATAAGGTGTTACAAAAAATGGAG | | |
| | | | AGCTCTTATTTTTGTACAGATTCTG | | |
| | | | TCGTTTTTGTTTTATTTGTGT | | |
| | | | GAGATTTATGGAAATACACTAAAT | | |
| | | | GAGTAATTCAGGTTCACTACATTT | | |
| | | | ATTACAAAGTGAAATCAGGGGA | | |
| | | | TATTCATTTGTAAATTTTATTCTTA | | |
| | | | GTGAATGAACTGTATAATTTTTTTT | | |
| | | | ATCAGGAGAGCACTTATAAA | | |
| | | | ATTCAATTTATAAAGATCATATAC | | |
| | | | CCAAATCATAAAGATTTAGTTGAT | | |
| | | | ACATTAACACTAAGATACTCTG | | |
| | | | ATTTTTAGCCGAACTAAACAAAGT | | |
| | | | GCTTCTACTGAGAGGCCTTTATACC | | |
| | | | ACCATGTACAGTAACTCTAAG | | |
| | | | TGAATACGGAAGACCTTGGTTTTG | | |
| | | | AAATTCTGCCACCTTGTTTCTCCCT | | |
| | | | GCTCATGAGGTCGCACCTTTT | | |
| | | | GCTCTTGCTGCTAATTCCCCATTCG | | |
| | | | TAGTGGGTGTAATGCCAGGTGGAA | | |
| | | | TGGTTTCAACAAGTCAGGTGA | | |
| | | | AAACCATCCTTTATTGTTGCTGGCA | | |
| | | | CAACTTGATATATAGTCTGACTCA | | |
| | | | GAACTGAAGCTCACATCTCAA | | |
| | | | ATTCATTTCATGCCAGTAAATGTG | | |
| | | | GCAAAGAGAAGAAAGGCCCAAGA | | |
| | | | GCGAGACAAGAAGAATGGAGAAG | | |
| | | | GGGGCAGCCAAGAAGAACTTCTGG | | |
| | | | GTTCAGGGTACTGTTTATTTGCTCC | | |
| | | | TTCTCTTCATGCCTGTGGCTG | | |
| | | | GATGTCCCACAACACTATAACAAA | | |
| | | | TATAAGTCAAGCCCTTTGTGTTAA | | |
| | | | GCAAGAACTACAGACTCCATCT | | |
| | | | TTTCACCCAAATCATGAATGACCA | | |
| | | | ATAAAAAGCAAGTTATTCCAGAGG | | |
| | | | AAGAAGCAGCCCTTGAAATGTT | | |
| | | | AAGGCTTAGGCTTGAAAGGTGAAG | | |
| | | | AGCAGGAATTCTCTCTTTCAAATCC | | |
| | | | TAGAGCATAAACCCATGTGTG | | |
| | | | GCCAAGTGAGATCAGCCCTCAAGG | | |
| | | | GCACATGCCAAGGGCAGAGCAGC | | |
| | | | CCATGTAGACAGCTTCGGAGGGC | | |
| | | | ATGGGGGTGTAGGGAGTTCGGGGGT | | |
| | | | AGCTCCTCATTAACTATTTGTTGGG | | |
| | | | TGAGTAAAGGGGTGAGGCTCA | | |
| | | | GTGGCAGGTACCTCTGCAATGACA | | |
| | | | AGCTGCCTCCCCTCTATGTGTTTAG | | |
| | | | CATATGTTATTAGAACATGTC | | |
| | | | CGACACCCCTACCGCTGCCATTTG | | |
| | | | GGCCCTTTAATAAAGCCAAGTAGA | | |
| | | | GAAATCTGGCAATAAAAGGCAA | | |
| | | | ATGTAAGCATGCTTTCTTTAAGAC | | |
| | | | GCATCATAAATGGTTTTCTTTAAGT | | |
| | | | GAATGGAAGAGTTTGACAGAG | | |
| | | | ATACACCTTTGTAAGAAAACATTA | | |
| | | | AGAATGCTGGCTGGCTGTGGTGCC | | |
| | | | TCACACCTGTATTCCCAGCACT | | |
| | | | TTGGGAGGCCTAGGCAGGAGGATT | | |
| | | | GCTTGAGCCTGGGACTTCGAGACC | | |
| | | | AGACTGGGAAACATGGCAAAAT | | |
| | | | CCCATCTCTACAACAAAAATACAA | | |
| | | | AAATTAGCCAAGTGCGGTGGTGTG | | |
| | | | CCTGTAGTCCTAGTTACTTGGG | | |
| | | | AGGCTGAGGTGGGAGAATCACCTG | | |
| | | | AGCCCAGGAGGTGGAGGCTGCAGT | | |
| | | | GAGCCATGCCAATGCACTCCAG | | |
| | | | TCTGGGCAACAGAGTGAGACCCTG | | |
| | | | TCTCAAAAATAAATAAATAAATAA | | |
| | | | ATGAATAAAGAGAATGCTAATC | | |
| | | | ATTTCTGGGTTCACTGCGACTCACT | | |
| | | | GTAGTGCTGGGGATCCCCCTTCTA | | |
| | | | ACACTGGAACTGAAAGACAGT | | |
| | | | GATGAAAGCTATGTCAAGCATTCA | | |
| | | | TTATTCTGAAGAGGAGGAGAAATG | | |
| | | | CCACATACCTTTCCCATGGGAC | | |
| | | | CTGTGGTGGAATGAATCCATACTT | | |
| | | | CTGCCTCACTTCGAGCAGACTTTTG | | |
| | | | TTCTCGGCGCTCCTCACGATG | | |
| | | | GAGTTTCATGCTTCATTTTCACATC | | |
| | | | TCTCTGCACAATTAGATTGGGAGC | | |
| | | | TCCTTGAGGGCAGAGTACGTG | | |
| | | | CCTTAATCTTTATCTTTGTAATGCC | | |
| | | | ACAATGAACAGAGTGCCTCCTGGT | | |
| | | | ACACTGTAGGAGCTTAAGAAA | | |
| | | | TACTCACTGAATGCATGAATGAAT | | |
| | | | GAATGAACAAATGAAGGAATGACT | | |
| | | | AAGGATGTTTGTAGTGCTATAA | | |
| | | | TATAGAATGGGATTTACTCTGCTTT | | |
| | | | ACCAGTTAGTTTCATAATAAACAA | | |
| | | | ATAGTCTGTA (SEQ ID NO: 13) | | |
| TNFSF10 | TNF | NM_003810.4 | GACCGGCTGCCTGGCTGACTTACA | NP_003801.1 | MAMMEVQGGPSLGQT |
| | Super- | | CCAGTCAGACTCTGACAGGATCAT | | CVLIVIFTVLLQSLCVAV |
| | family | | GGCTATGATGGAGGTCCAGGGG | | TYVYFTNELKQ |
| | Member | | GGACCCAGCCTGGGACAGACCTGC | | MQDKYSKSGIACFLKE |
| | 10 | | GTGCTGATCGTGATCTTCACACTG | | DDSYWDPNDEESMNSP |
| | | | CTCCTGCAGTCTCTCTGTGTGG | | CWQVKWQLRQLVRKM |
| | | | CTGTAACTTACGTGTACTTTACCAA | | ILRTSEETIST |
| | | | CGAGCTGAAGCAGATGCAGGACA | | VQEKQQNISPLVRERGP |
| | | | AGTACTCCAAAAGTGGCATTGC | | QRVAAHITGIRGRSNTL |
| | | | TTGTTTCTTAAAAGAAGATGACAG | | SSPNSKNEKALGRKINS |
| | | | TTATTGGGACCCCAATGACGAAGA | | WESSRSG |
| | | | GAGTATGAACAGCCCCTGCTGG | | HSFLSNLHLRNGELVIH |
| | | | CAAGTCAAGTGGCAACTCCGTCAG | | EKGFYYIYSQTYFREQE |
| | | | CTCGTTAGAAAGATGATTTTGAGA | | EIKENTKNDKQMVQYI |
| | | | ACCTCTGAGGAAACCATTTCTA | | YKYTSYPD |
| | | | CACTTCAACAAAAGCAACAAAATA | | PILLMKSARNSCWSKD |
| | | | TTTCTCCCCTAGTGAGAGAAAGAG | | AEYGLYSIYQGGIFELK |
| | | | GTCCTCAGAGAGTACCAGCTCA | | ENDRIFVSVTNEHLIDM |
| | | | CATAACTGGGACCAGAGGAAGAA | | DHEASFFG |
| | | | GCAACACATTGTCTTCTCCAAACT | | AFLVG (SEQ ID NO: 16) |
| | | | CCAAGAATGAAAAGGCTCTGGGC | | |
| | | | CGCAAAATAAACTCCTGGGAATCA | | |
| | | | TCAAGGAGTGGGCATTCATTCCTG | | |
| | | | AGCAACTTGCACTTGAGGAATG | | |
| | | | GTGAACTGGTCATCCATGAAAAAG | | |
| | | | GGTTTTACTACATCTATTCCCAAAC | | |
| | | | ATACTTTCGATTTCAGGAGGA | | |
| | | | AATAAAAGAAAACACAAAGAACG | | |
| | | | ACAAACAAATGGTCCAATATATTT | | |
| | | | ACAAATACACAAGTTATCCTGAC | | |
| | | | CCTATATTGTTGATGAAAAGTGCT | | |
| | | | AGAAATAGTTGTTGGTCTAAAGAT | | |
| | | | CCAGAATATGGACTCTATTCCA | | |
| | | | TCTATCAAGGGGGAATATTTGAGC | | |
| | | | TTAAGGAAAATGACAGAATTTTTG | | |
| | | | TTTCTGTAACAAATGAGCACTT | | |
| | | | GATAGACATGGACCATGAAGCCAG | | |
| | | | TTTTTTTGGGGCCTTTTTAGTTGGC | | |
| | | | TAACTGACCTGGAAAGAAAAA | | |
| | | | GCAATAACCTCAAAGTGACTATTC | | |
| | | | AGTTTTCAGGATGATACACTATGA | | |
| | | | AGATGTTTCAAAAAATCTGACC | | |
| | | | AAAACAAACAAACAGAAAACACA | | |
| | | | AAACAAAAAAACCTCTATGCAATC | | |
| | | | TGAGTAGAGCAGCCACAACCAAA | | |
| | | | AAATTCTACAACACACACTGTTCT | | |
| | | | GAAAGTGACTCACTTATGCCAAGA | | |
| | | | GAATGAAATTGCTGAAAGATCT | | |
| | | | TTCAGGACTCTACCTCATATCAGTT | | |
| | | | TGCTAGCAGAAATCTAGAAGACTG | | |
| | | | TCAGCTTCCAAACATTAATGC | | |
| | | | AATGGTTAACATCTTCTGTCTTTAT | | |
| | | | AATCTACTCCTTGTAAAGACTGTA | | |
| | | | GAAGAAAGAGCAACAATCCAT | | |
| | | | CTCTCAAGTAGTGTATCACAGTAG | | |
| | | | TAGCCTCCAGGTTTCCTTAAGGGA | | |
| | | | CAACATCCTTAAGTCAAAAGAG | | |
| | | | AGAAGAGGCACCACTAAAAGATC | | |
| | | | GCAGTTTGCCTGGTGCAGTGGCTC | | |
| | | | ACACCTGTAATCCCAACATTTTG | | |
| | | | CGAACCCAAGGTGGGTAGATCACG | | |
| | | | AGATCAAGAGATCAAGACCATAGT | | |
| | | | GACCAACATACTGAAACCCCAT | | |
| | | | CTCTACTGAAAGTACAAAAATTAG | | |
| | | | CTGGGTGTGTTGGCACATGCCTGT | | |
| | | | AGTCCCAGCTACTTGAGAGGCT | | |
| | | | GAGGCAAGAGAATTGTTTGAACCC | | |
| | | | GGGAGGCAGAGGTTGCAGTGTGGT | | |
| | | | GAGATCATGCCACTACACTCCA | | |
| | | | GCCTGGCGACAGAGCGAGACTTGG | | |
| | | | TTTCAAAAAAAAAAAAAAAAAAA | | |
| | | | ACTTCAGTAAGTACGTGTTATTT | | |
| | | | TTTTCAATAAAATTCTATTACAGTA | | |
| | | | TGTCATGTTTGCTGTAGTGCTCATA | | |
| | | | TTTATTGTTGTTTTTGTTTT | | |
| | | | AGTACTCACTTGTTTCATAATATCA | | |
| | | | AGATTACTAAAAATGGGGGAAAA | | |
| | | | GACTTCTAATCTTTTTTTCATA | | |
| | | | ATATCTTTGACACATATTACAGAA | | |
| | | | GAAATAAATTTCTTACTTTTAATTT | | |
| | | | AATATGA (SEQ ID NO: 15) | | |
| | | NM_001190942.2 | GACCGGCTGCCTGGCTGACTTACA | NP_001177871.1 | MAMMEVQGGPSLGQT |
| | | | CCAGTCAGACTCTGACAGGATCAT | | CVLIVIFTVLLQSLCVAV |
| | | | GGCTATGATGGAGGTCCAGGGG | | TYVYFTNELKQ |
| | | | GGACCCAGCCTGGGACAGACCTGC | | MQDKYSKSCIACFLKE |
| | | | GTGCTGATCGTGATCTTCACAGTG | | DDSYWDPNDEESMNSP |
| | | | CTCCTGCAGTCTCTCTGTGTGG | | CWQVKWQLRQLVRKT |
| | | | CTGTAACTTACGTGTACTTTACCAA | | PRMKRLWAAK (SEQ ID |
| | | | CGAGCTGAAGCAGATGCAGGACA | | NO: 18) |
| | | | AGTACTCCAAAAGTGGCATTGC | | |
| | | | TTGTTTCTTAAAAGAAGATGACAG | | |
| | | | TTATTGGGACCCCAATGACGAAGA | | |
| | | | GAGTATGAACAGCCCCTGCTGG | | |
| | | | CAAGTCAAGTGGCAACTCCGTCAG | | |
| | | | CTCGTTAGAAAGACTCCAAGAATG | | |
| | | | AAAAGGCTCTGGGCCGCAAAAT | | |
| | | | AAACTCCTGGGAATCATCAAGGAG | | |
| | | | TGGGCATTCATTCCTGAGCAACTT | | |
| | | | GCACTTGAGGAATGGTGAACTG | | |
| | | | GTCATCCATGAAAAAGGGTTTTAC | | |
| | | | TACATCTATTCCCAAACATACTTTC | | |
| | | | GATTTCAGGAGGAAATAAAAG | | |
| | | | AAAACACAAAGAACGACAAACAA | | |
| | | | ATGGTCCAATATATTTACAAATAC | | |
| | | | ACAAGTTATCCTGACCCTATATT | | |
| | | | GTTGATGAAAAGTGCTAGAAATAG | | |
| | | | TTGTTGGTCTAAAGATGCAGAATA | | |
| | | | TGGACTCTATTCCATCTATCAA | | |
| | | | GGGGGAATATTTGAGCTTAAGGAA | | |
| | | | AATGACAGAATTTTTGTTTCTGTAA | | |
| | | | CAAATGAGCACTTGATAGACA | | |
| | | | TGGACCATGAAGCCAGTTTTTTTG | | |
| | | | GGGCCTTTTTAGTTGGCTAACTGAC | | |
| | | | CTGGAAAGAAAAAGCAATAAC | | |
| | | | CTCAAAGTGACTATTCAGTTTTCAG | | |
| | | | GATGATACACTATGAAGATGTTTC | | |
| | | | AAAAAATCTGACCAAAACAAA | | |
| | | | CAAACAGAAAACAGAAAACAAAA | | |
| | | | AAACCTCTATGCAATCTGAGTAGA | | |
| | | | GCAGCCACAACCAAAAAATTCTA | | |
| | | | CAACACACACTGTTCTGAAAGTGA | | |
| | | | CTCACTTATCCCAAGAGAATGAAA | | |
| | | | TTGCTGAAAGATCTTTCACGAC | | |
| | | | TCTACCTCATATCAGTTTGCTAGCA | | |
| | | | GAAATCTAGAAGACTGTCAGCTTC | | |
| | | | CAAACATTAATGCAATGGTTA | | |
| | | | ACATCTTCTGTCTTTATAATCTACT | | |
| | | | CCTTGTAAAGACTGTAGAAGAAAG | | |
| | | | AGCAACAATCCATCTCTCAAG | | |
| | | | TAGTGTATCACAGTAGTAGCCTCC | | |
| | | | AGGTTTCCTTAAGGGACAACATCC | | |
| | | | TTAAGTCAAAAGAGAGAAGAGG | | |
| | | | CACCACTAAAAGATCGCAGTTTGC | | |
| | | | CTGGTGCAGTGGCTCACACCTGTA | | |
| | | | ATCCCAACATTTTGGGAACCCA | | |
| | | | AGGTGGGTAGATCACGAGATCAAG | | |
| | | | AGATCAAGACCATAGTGACCAACA | | |
| | | | TAGTCAAACCCCATCTCTACTG | | |
| | | | AAAGTACAAAAATTAGCTGGGTGT | | |
| | | | GTTGGCACATGCCTGTAGTCCCAG | | |
| | | | CTACTTGAGAGGCTGAGGCAAG | | |
| | | | AGAATTGTTTGAACCCGGGAGGCA | | |
| | | | GAGGTTGCAGTGTGGTGAGATCAT | | |
| | | | GCCACTACACTCCAGCCTGGCG | | |
| | | | ACAGAGCGAGACTTGGTTTCAAAA | | |
| | | | AAAAAAAAAAAAAAAACTTCACT | | |
| | | | AAGTACGTGTTATTTTTTTCAAT | | |
| | | | AAAATTCTATTACAGTATGTCATGT | | |
| | | | TTGCTGTAGTGCTCATATTTATTGT | | |
| | | | TGTTTTTGTTTTAGTACTCA | | |
| | | | CTTGTTTCATAATATCAAGATTACT | | |
| | | | AAAAATGGGGGAAAAGACTTCTAA | | |
| | | | TCTTTTTTTCATAATATCTTT | | |
| | | | GACACATATTACAGAAGAAATAAA | | |
| | | | TTTCTTACTTTTAATTTAATATGA | | |
| | | | (SEQ ID NO: 17) | | |
| | | NM_001190943.2 | GACCGGCTGCCTGGCTGACTTACA | NP_001177872.1 | MAMMEVQGGPSLGQT |
| | | | GCAGTCAGACTCTGACAGGATCAT | | CVLIVIFTVLLQSLCVAV |
| | | | GGCTATGATGGAGGTCCAGGGG | | TYVYFTNELKQFAEND |
| | | | GGACCCAGCCTGGGACAGACCTGC | | CQRLMSCQQTGSLIPS |
| | | | GTGCTGATCGTGATCTTCACAGTG | | (SEQ ID NO: 20) |
| | | | CTCCTGCAGTCTCTCTGTGTGG | | |
| | | | CTGTAACTTACGTGTACTTTACCAA | | |
| | | | CGAGCTGAAGCAGTTTGCAGAAAA | | |
| | | | TGATTGCCAGAGACTAATGTC | | |
| | | | TGGGCAGCAGACAGGGTCATTGCT | | |
| | | | GCCATCTTGAAGTCTACCTTGCTGA | | |
| | | | GTCTACCCTGCTGACCTCAAG | | |
| | | | CCCCATCAAGGACTGGTTGACCCT | | |
| | | | GGCCTAGACAACCACCGTGTTTGT | | |
| | | | AACAGCACCAAGAGCAGTCACC | | |
| | | | ATGGAAATCCACTTTTCAGAACCA | | |
| | | | AGGGCTTCTGGAGCTGAAGAACAG | | |
| | | | CCACCCAGTGCAAGAGCTTTCT | | |
| | | | TTTCAGAGGCACGCAAATGAAAAT | | |
| | | | AATCCCCACACGCTACCTTCTGCC | | |
| | | | CCCAATCCCCAAGTGTGGTTAG | | |
| | | | TTAGAGAATATAGCCTCAGCCTAT | | |
| | | | GATATGCTGCAGGAAACTCATATT | | |
| | | | TTGAAGTGGAAAGGATGGGAGG | | |
| | | | AGGCGGGGGAGACGTATCGTATTA | | |
| | | | ATTATCATTCTTGGAATAACCACA | | |
| | | | GCACCTCACGTCAACCCGCCAT | | |
| | | | GTGTCTAGTCACCAGCATTGGCCA | | |
| | | | AGTTCTATAGGAGAAACTACCAAA | | |
| | | | ATTCATGATGCAAGAAACATGT | | |
| | | | GAGGGTGGAGAGAGTGACTGGGG | | |
| | | | CTTCCTCTCTGGATTTCTATTGTTC | | |
| | | | AGAAATCAATATTTATGCATAA | | |
| | | | AAAGGTCTAGAAAGAGAAACACC | | |
| | | | AAAATGACAATGTGATCTCTAGAT | | |
| | | | GGTATGATTATGGGTACTTTTTT | | |
| | | | TCCTTTTTATTTTTCTATATTTTACA | | |
| | | | AATTTTCTACAGGGAATGTTATAA | | |
| | | | AAATATCCATGCTATCCATG | | |
| | | | TATAATTTTCATACAGATTTAAAG | | |
| | | | AACACAGCATTTTTATATAGTCTTA | | |
| | | | TGAGAAAACAACCATACTCAA | | |
| | | | AATTATGCACACACACAGTCTGAT | | |
| | | | CTCACCCCTGTAAACAAGAGATAT | | |
| | | | CATCCAAAGGTTAAGTAGGAGG | | |
| | | | TGAGAATATAGCTGCTATTAGTGG | | |
| | | | TTGTTTTGTTTTGTTTTTGTGATTTA | | |
| | | | CTTATTTAGTTTTTGGAGGG | | |
| | | | TTTTTTTTTTCTTTTAGAAAAGTGT | | |
| | | | TCTTTACTTTTCCATGCTTCCCTGC | | |
| | | | TTGCCTGTGTATCCTGAATG | | |
| | | | TATCCAGGCTTTATAAACTCCTGG | | |
| | | | GTAATAATGTAGCTACATTAACTT | | |
| | | | GTTAACCTCCCATCCACTTATA | | |
| | | | CCCAGGACCTTACTCAATTTTCCA | | |
| | | | GGTTC (SEQ ID NO: 19) | | |
| LY96 | Lymphocyte | NM_015364.5 | GATTAGTTACTGATCCTCTTTGCAT | NP_056179.4 | MLPFLFFSTLFSSIFTEA |
| | Antigen | | TTGTAAAGCTTTGGAGATATTGAA | | QKQYWVCNSSDASISY |
| | 96 | | TCATGTTACCATTTCTGTTTT | | TYCDKMQYPI |
| | | | TTTCCACCCTGTTTTCTTCCATATTT | | SINVNPCIELKRSKGLLH |
| | | | ACTGAAGCTCAGAAGCAGTATTGG | | IFYIPRRDLKQLYFNLYI |
| | | | GTCTGCAACTCATCCGATGC | | TVNTMNLPKRKEVICR |
| | | | AAGTATTTCATACACCTACTGTCAT | | GSDDDY |
| | | | AAAATGCAATACCCAATTTCAATT | | SFCRALKGETVNTTISFS |
| | | | AATGTTAACCCCTGTATAGAA | | FKGIKFSKGKYKCVVEA |
| | | | TTGAAAAGATCCAAAGGATTATTG | | ISGSPEEMLFCLEFVILH |
| | | | CACATTTTCTACATTCCAAGGAGA | | QPNSN (SEQ ID NO: 22) |
| | | | GATTTAAAGCAATTATATTTTCA | | |
| | | | ATCTCTATATAACTGTCAACACCAT | | |
| | | | GAATCTTCCAAAGCGCAAAGAAGT | | |
| | | | TATTTGCCGAGGATCTGATGA | | |
| | | | CGATTACTCTTTTTGCAGAGCTCTG | | |
| | | | AAGGGAGAGACTGTGAATACAAC | | |
| | | | AATATCATTCTCCTTCAAGGGA | | |
| | | | ATAAAATTTTCTAAGGGAAAATAC | | |
| | | | AAATGTGTTGTTGAAGCTATTTCTG | | |
| | | | GGAGCCCAGAAGAAATGCTCT | | |
| | | | TTTGCTTGGAGTTTGTCATCCTACA | | |
| | | | CCAACCTAATTCAAATTAGAATAA | | |
| | | | ATTGAGTATTTAAAAAAAAA (SEQ | | |
| | | | ID NO: 21) | | |
| | | NM_001195797.1 | AGAAATCATGTGACTGATGACTAA | NP_001182726.1 | MLPFLFFSTLFSSIFTEA |
| | | | GTTAAATCTTTTCTGCTTACTGAAA | | QKQYWVCNSSDASISY |
| | | | AGGAAGAGTCTGATGATTAGT | | TYCGRDIKQL |
| | | | TACTGATCCTCTTTGCATTTGTAAA | | YFNLYITVNTMNLPKRK |
| | | | GCTTTGGAGATATTGAATCATGTT | | EVICRGSDDDYSFCRAL |
| | | | ACCATTTCTGTTTTTTTCCAC | | KGETVNTTISFSFKGIKF |
| | | | CCTGTTTTCTTCCATATTTACTGAA | | SKGKYK |
| | | | GCTCAGAAGCAGTATTGGGTCTGC | | CVVEAISGSPEEMLFCL |
| | | | AACTCATCCGATGCAAGTATT | | EFVILHQPNSN (SEQ ID |
| | | | TCATACACCTACTGTGGGAGAGAT | | NO: 24) |
| | | | TTAAAGCAATTATATTTCAATCTCT | | |
| | | | ATATAACTGICAACACCATGA | | |
| | | | ATCTTCCAAAGCGCAAAGAAGTTA | | |
| | | | TTTGCCGAGGATCTGATGACGATT | | |
| | | | ACTCTTTTTGCAGAGCTCTGAA | | |
| | | | GGGAGAGACTGTGAATACAACAAT | | |
| | | | ATCATTCTCCTTCAAGGGAATAAA | | |
| | | | ATTTTCTAAGGGAAAATACAAA | | |
| | | | TGTGTTGTTGAAGCTATTTCTGGGA | | |
| | | | GCCCAGAAGAAATGCTCTTTTGCT | | |
| | | | TGGAGTTTGTCATCCTACACC | | |
| | | | AACCTAATTCAAATTAGAATAAAT | | |
| | | | TGAGTATTTAAAAAAAAAAAAAAA | | |
| | | | AAAAAAAAAAAAAA (SEQ ID NO: | | |
| | | | 23) | | |
| QPCT | Gluta- | NM_012413.4 | AGTCGACCCAAGGGTGGAGAAGA | NP_036545.1 | MAGGRHRRVVGTLHLL |
| | methyl- | | GGGAAGGCGAAGGACGCGCGTTC | | LLVAALPWASRGVSPS |
| | Peptide | | CCGGGCTCCTGACCGCCAGCGGCC | | ASAWPEEKNYHQ |
| | Cyclo- | | CGGGGAACCCGCTCCCAGACAGAC | | PAILNSSALRQIAEGTSIS |
| | trans- | | TCGGAGAGATGGCAGGCGGAAGA | | EMWQNDLQPLLIERYP |
| | ferace | | CACCGGCGCGTCGTGGGCACCCT | | GSPGSYAARQHIMQRIQ |
| | | | CCACCTGCTGCTGCTGGTGGCCGC | | RLQADW |
| | | | CCTGCCCTGGGCATCCAGGGGGGT | | VLEIDTFLSQTPYGYRSF |
| | | | CAGTCCGAGTGCCTCAGCCTGG | | SNHSTLNPTAKRHLVLA |
| | | | CCAGAGGAGAAGAATTACCACCA | | CHYDSKYFSHWNNRVF |
| | | | GCCAGCCATTTTGAATTCATCGGCT | | VGATDS |
| | | | CTTCGGCAAATTGCAGAAGGCA | | AVPCAMMLELARALDK |
| | | | CCAGTATCTCTGAAATGTGGCAAA | | KLLSLKTVSDSKPDLSL |
| | | | ATGACTTACAGCCATTGCTGATAG | | QLIFFDGEEAFLHWSPQ |
| | | | AGCGATACCCGGGATCCCCTGG | | DSLYGSRH |
| | | | AAGCTATGCTGCTCGTCAGCACAT | | LAAKMASTPHPPGARG |
| | | | CATGCAGCGAATTCAGAGGCTTCA | | TSQLHGMDLLVLLDLIG |
| | | | GGCTGACTGGGTCTTGGAAATA | | APNPTFPNFFPNSARWF |
| | | | GACACCTTCTTGAGTCAGACACCC | | ERLQAIEH |
| | | | TATGGGTACCGGTCTTTCTCAAATA | | ELHELGLLKDHSLEGRY |
| | | | TCATCAGCACCCTCAATCCCA | | FQNYSYGGVIQDDHIPF |
| | | | CTGCTAAACGACATTTGGTCCTCG | | LRRGVPVLHLIPSPFPEV |
| | | | CCTGCCACTATGACTCCAAGTATTT | | WHTMDD |
| | | | TTCCCACTGGAACAACAGAGT | | NEENLDESTIDNLNKILQ |
| | | | GTTTGTAGGAGCCACTGATTCAGC | | VFVLEYLHL (SEQ ID |
| | | | CGTGCCATGTGCAATGATGTTGGA | | NO: 26) |
| | | | ACTTGCTCGTGCCTTAGACAAG | | |
| | | | AAACTCCTTTCCTTAAAGACTGTTT | | |
| | | | CAGACTCCAAGCCAGATTTGTCAC | | |
| | | | TCCAGCTGATCTTCTTTGATG | | |
| | | | GTGAAGAGGCTTTTCTTCACTGGTC | | |
| | | | TCCTCAAGATTCTCTCTATGGGTCT | | |
| | | | CGACACTTAGCTGCAAAGAT | | |
| | | | GGCATCGACCCCGCACCCACCTGG | | |
| | | | AGCGAGAGGCACCAGCCAACTGC | | |
| | | | ATGGCATGGATTTATTGGTCTTA | | |
| | | | TTGGATTTGATTGGAGCTCCAAAC | | |
| | | | CCAACGTTTCCCAATTTTTTTCCAA | | |
| | | | ACTCAGCCAGGTGGTTCGAAA | | |
| | | | GACTTCAAGCAATTGAACATGAAC | | |
| | | | TTCATGAATTGGGTTTGCTCAAGG | | |
| | | | ATCACTCTTTGGAGGGGCGGTA | | |
| | | | TTTCCAGAATTACACTTATGGAGG | | |
| | | | TGTGATTCAGGATGACCATATTCC | | |
| | | | ATTTTTAAGAAGAGGTGTTCCA | | |
| | | | GTTCTGCATCTGATACCGTCTCCTT | | |
| | | | TCCCTGAAGTCTGGCACACCATGG | | |
| | | | ATGACAATGAAGAAAATTTGG | | |
| | | | ATGAATCAACCATTGACAATCTAA | | |
| | | | ACAAAATCCTACAAGTCTTTGTGTT | | |
| | | | GGAATATCTTCATTTGTAATA | | |
| | | | CTCTGATTTAGTTTAGGATAATTGG | | |
| | | | TTCTAGAATTGAATTCAAAAGTCA | | |
| | | | AGGCATCATTTAAAATAATCT | | |
| | | | GATTTCAGACAAATGCTGTGTGGA | | |
| | | | AACATCTATCCTATAGATCATCCTA | | |
| | | | TTCTTATGTGTCTTTGGTTAT | | |
| | | | CAGATCAATTACAGAATAATTGTG | | |
| | | | TTGTGATATTGTGTCCTAAATTGCT | | |
| | | | CATTAATTTTTATTTACAGAT | | |
| | | | TGAAAAAGAGGGACCGTGTAAAG | | |
| | | | AAAATGGAAAATAAATATCTTTCA | | |
| | | | AAGACTCTTTTAGATAAACACGA | | |
| | | | TGAGGCAAAATCAGGTTCATTCAT | | |
| | | | TCAACGATAGTTTCTCAACAGTAC | | |
| | | | TTAAATAGCGGTTGGAAAACGT | | |
| | | | AGCCTTCATTTTATGATTTTTTCAT | | |
| | | | ATGTGGAAATCTATTACATGTAAT | | |
| | | | ACAAAACAAACATGTAGTTTG | | |
| | | | AAGGCGGTCAGATTTCTTTGAGAA | | |
| | | | ATCTTTGTAGAGTTAATTTTATGGA | | |
| | | | AATTAAAATCAGAATTAAATG | | |
| | | | CTA (SEQ ID NO: 25) | | |
| KYNU | Kynum- | NM_003937.3 | ACATTTTCAAGGAATTCTTGAGAG | NP_003928.1 | MEPSSLELPADTVQRIA |
| | reninase | | GTTCTTGGAGAGATTCTGGGAGCC | | AELKCHPTDERVALHL |
| | | | AAACACTCCATTGGGATCCTAG | | DEEDKLRHFRE |
| | | | CTGTTTTAGAGAACAACTTGTAAT | | CFYIPKIQDLPPVDLSLV |
| | | | GGAGCCTTCATCTCTTGAGCTGCC | | NKDENAIYFLGNSLGLQ |
| | | | GGCTGACACAGTGCAGCGCATT | | PKMVKTYLEEELDKWA |
| | | | GCGGCTGAACTCAAATGCCACCCA | | KIAAYGH |
| | | | ACGGATGAGAGGGTGGCTCTCCAC | | EVGKRPWITGDESIVGL |
| | | | CTAGATGAGGAAGATAAGCTGA | | MKDIVGANEKEIALMN |
| | | | CGCACTTCAGGGAGTGCTTTTATA | | ALTVNLHLLMLSFFKPT |
| | | | TTCCCAAAATACAGGATCTGCCTC | | PKRYKILL |
| | | | CAGTTGATTTATCATTAGTGAA | | EAKAFPSDHYAIESQLQ |
| | | | TAAAGATGAAAATGCCATCTATTT | | LHGLNIEESMRMIKPRE |
| | | | CTTGGGAAATTCTCTTGGCCTTCAA | | GEETLRIEDILEVIEKEG |
| | | | CCAAAAATGGTTAAAACATAT | | DSIAVI |
| | | | CTTGAAGAAGAACTAGATAAGTGG | | LESGVHFYTGQHFNIPAI |
| | | | GCCAAAATAGCAGCCTATGGTCAT | | TKAGQAKGCYVGFDLA |
| | | | GAAGTGGGGAAGCGTCCTTGGA | | HAVGNVELYLHDWGV |
| | | | TTACAGGAGATGAGAGTATTGTAG | | DFACWCSYK |
| | | | GCCTTATGAAGGACATTGTAGGAG | | YLNAGAGGIAGAFIHEK |
| | | | CCAATGAGAAAGAAATAGCCCT | | HAHTIKPALVGWEGHE |
| | | | AATGAATGCTTTGACTGTAAATTT | | LSTRFKMDNKLQLIPGV |
| | | | ACATCTTCTAATGTTATCATTTTTT | | CGFRISNP |
| | | | AAGCCTACGCCAAAACGATAT | | PILLVCSLHASLEIFKQA |
| | | | AAAATTCTTCTAGAAGCCAAAGCC | | TMKALRKKSVLLTGYL |
| | | | TTCCCTTCTGATCATTATGCTATTG | | EYLIKHNYGKDKAATK |
| | | | AGTCACAACTACAACTTCACG | | KPVVNIIT |
| | | | GACTTAACATTGAAGAAAGTATGC | | PSHVEERGCQLTITFSVP |
| | | | GGATGATAAAGCCAAGAGAGGGG | | NKDVFQELEKRGVVCD |
| | | | GAAGAAACCTTAAGAATAGAGGA | | KRNPNGIRVAPVPLYNS |
| | | | TATCCTTCAAGTAATTGAGAAGGA | | FHDVYKF |
| | | | AGGAGACTCAATTGCAGTGATCCT | | TNLLTSILDSAETKN |
| | | | GTTCAGTGGGGTGCATTTTTAC | | (SEQ ID NO: 28) |
| | | | ACTGGACAGCACTTTAATATTCCT | | |
| | | | GCCATCACAAAAGCTGGACAAGCG | | |
| | | | AAGGGTTGTTATGTTGGCTTTG | | |
| | | | ATCTAGCACATGCAGTTGGAAATG | | |
| | | | TTGAACTCTACTTACATGACTGGG | | |
| | | | GAGTTGATTTTGCCTGCTGGTG | | |
| | | | TTCCTACAAGTATTTAAATGCAGG | | |
| | | | AGCAGGAGGAATTGCTGGTGCCTT | | |
| | | | CATTCATGAAAACCATGCCCAT | | |
| | | | ACGATTAAACCTGCATTAGTGGGA | | |
| | | | TGGTTTGGCCATGAACTCAGCACC | | |
| | | | AGATTTAACATGGATAACAAAC | | |
| | | | TGCAGTTAATCCCTGGGGTCTGTG | | |
| | | | GATTCCGAATTTCAAATCCTCCCAT | | |
| | | | TTTCTTGGTCTGTTCCTTGCA | | |
| | | | TGCTAGTTTAGAGATCTTTAAGCA | | |
| | | | AGCGACAATGAAGGCATTGCGGAA | | |
| | | | AAAATCTGTTTTGCTAACTGGC | | |
| | | | TATCTGGAATACCTGATCAAGCAT | | |
| | | | AACTATGGCAAAGATAAAGCAGCA | | |
| | | | ACCAAGAAACCAGTTGTGAACA | | |
| | | | TAATTACTCCGTCTCATGTAGAGG | | |
| | | | AGCGGGGGTGCCAGCTAACAATAA | | |
| | | | CATTTTCTGTTCCAAACAAAGA | | |
| | | | TGTTTTCCAAGAACTAGAAAAAAG | | |
| | | | AGGAGTGGTTTGTGACAAGCGGAA | | |
| | | | TCCAAATGGCATTCGAGTGGCT | | |
| | | | CCAGTTCCTCTCTATAATTCTTTCC | | |
| | | | ATGATGTTTATAAATTTACCAATCT | | |
| | | | GCTCACTTCTATACTTGACT | | |
| | | | CTGCAGAAACAAAAAATTACCAGT | | |
| | | | GTTTTCTAGAACAACTTAAGCAAA | | |
| | | | TTATACTGAAAGCTGCTGTGGT | | |
| | | | TATTTCAGTATTATTCGATTTTTAA | | |
| | | | TTATTGAAAGTATGTCACCATTGA | | |
| | | | CCACATGTAACTAACAATAAA | | |
| | | | TAATATACCTTACAGAAAATCTGA | | |
| | | | TATAATTTTTCAGAGTCTGTGGCAC | | |
| | | | TAAGGAGTCCACAGGGCTGCC | | |
| | | | TAGGTGCTTTGTGTTTGGGGGACC | | |
| | | | AAAACTGTGTTGGTTCAAGTATTA | | |
| | | | TCTATACAGTCTCTATAAGCTG | | |
| | | | TCACATTTCATGGTCATTGAAATGT | | |
| | | | TTTATGTTGGTTTAATTTCTGATTT | | |
| | | | AACTGACAACTTCATAATGT | | |
| | | | ATGTGCAATTATTGTGTCAAATTTA | | |
| | | | GAAATATTACTTTAGCTTCAATTTA | | |
| | | | CCAAGGAGTTTCTTTGAAGC | | |
| | | | ATTGTAGTCTGATATATATATATAT | | |
| | | | ATATATATATATATATATATATATA | | |
| | | | TATATATATGTGTGTGTGTG | | |
| | | | TGTGTGTATATATATATATATATAT | | |
| | | | CATATATATATGATAGTGGCTTTCA | | |
| | | | AATTTTTTTGGCTACAATCC | | |
| | | | ACATTGCTCCTGCTGATCTGTAATA | | |
| | | | TCAGAAACCAGTATTTATGTGAAT | | |
| | | | ATATCAGAAATATTATTGATT | | |
| | | | CTAAGATATTTTATCATATTTTAAC | | |
| | | | ATCTTTGAAAGAGGACCCATCTTT | | |
| | | | CAATTTTCGATCAATAGTTTC | | |
| | | | TTACAGTCACCATTGGCCATCTTTC | | |
| | | | TCGTTACCATCTATGAAATTAGCAT | | |
| | | | GCATCTCAAATAAACAGTTA | | |
| | | | CCATCTTCTATTTGATAAAATAGTC | | |
| | | | TAAATAGCAAAAATAAAAGTTTTT | | |
| | | | ACAATTATTTGCCTGTGCTCT | | |
| | | | AATAGGTACTATTCTATTTTATCTC | | |
| | | | ATAAGAAATGTTGGAAACTCATTA | | |
| | | | TATTGATTTCCTTACCCACTC | | |
| | | | ATGGGCCCTAATTCACACTTTTTAA | | |
| | | | GAATGTTTCTTTCTTTAATGTTATC | | |
| | | | ATAATCTCTTACTTTTTAAA | | |
| | | | TGAGAACTTCCCCTAATATAAGAG | | |
| | | | CTTAGATATTATATTACTATGTTTC | | |
| | | | CATAGTAAATAAATAACCCCA | | |
| | | | AGATCTTTTTGGGGATTAGAGATA | | |
| | | | TAAGAAATATGTGCTCCATCTCTTG | | |
| | | | ACATCTTTATCTCAAATCTAT | | |
| | | | GGACCTTTCTTACCCACTGTGAAA | | |
| | | | AACCTAAAGTTACACTTAGCCCTG | | |
| | | | TTGGACTTACCTAGTTTTCAAT | | |
| | | | TGTTGATGCCACAATCATTATTTAT | | |
| | | | AAGTTGACAAAATAGTGTAGATTT | | |
| | | | GTATACATAGTCAACAAAAAG | | |
| | | | AGTGACATAATTATTGCCTCCAATT | | |
| | | | AAACAAGTTTGAATGAAATAAACA | | |
| | | | AACTTAGATAAACACTTCGGA | | |
| | | | TGGTAGACGTAAACAATAATATGT | | |
| | | | GGAACTCCAACATCAACACCTACC | | |
| | | | AATACCAGTAACTACTGATATT | | |
| | | | TATCATGTACTTACCATGTACCATG | | |
| | | | TATTGTGCTACATTACTCATGTTAT | | |
| | | | CTCCCTTAATTGAGTGGCTA | | |
| | | | CATACTGCTTTAGCAAATCTTCCTA | | |
| | | | CTGTAACTAATCCTCATACATGGA | | |
| | | | AGAGTTCTCAAAACCTTAAAA | | |
| | | | CTCATGCATAAGTGGATTCATATA | | |
| | | | CATATATAAAAATATATATAAATA | | |
| | | | TATATACTTTATATATATTTAT | | |
| | | | ATTTATATATTTATATATTTATATT | | |
| | | | TTAATATATTTATATAAATATATAT | | |
| | | | AAAGTATAATATATATAAAG | | |
| | | | TATAAATATATATATATTTATACTT | | |
| | | | TAAGTTCTTGGATACACGTGCAGA | | |
| | | | ACATGCAGGTTTGTTACATAG | | |
| | | | GTATACATGTGCCGTGGTGGATTG | | |
| | | | CTGCACCCATCAACCCGTCATCTA | | |
| | | | CATCAGGTATTTCTCCTAATGC | | |
| | | | TCACCCTCCTCTTATCCCCAACTAC | | |
| | | | CCAAAAGGACCTGGTGTGTGATGT | | |
| | | | TCCCCTCCCTGTGTTCATATG | | |
| | | | TTCTCATTGTTCAACTCTCACTTAT | | |
| | | | GGGTAAGAACATGCAGTTGTTTGAT | | |
| | | | TTTCTGTTCCTCTGTTAGTTT | | |
| | | | GCTGAGAATGATGGTTTCCAGCTT | | |
| | | | CATCCATGTCCCTGCAAAGGACAT | | |
| | | | GAACTCATTCTTTTTTATGGCT | | |
| | | | CCATAGTATTCCATGGTATATATGT | | |
| | | | GCCACATTTTCTTTATCCAGTCTAT | | |
| | | | CATTGATGGCCATTTGAGTT | | |
| | | | GGTTCCAAGTCTTCGCTATTGTGAA | | |
| | | | TAGTGCTGCAATGAACATATGTGT | | |
| | | | GCATGTGTCTTTATAGTAGAA | | |
| | | | TGATTTATAATCCTTAGGGTATACC | | |
| | | | CAGTAATGGGATTGCTGGGTTAAA | | |
| | | | TGGTATTTCTGGTTCTAGATC | | |
| | | | CTCGAGGAATTGCCACACTGTCTT | | |
| | | | CCACAATGGTTGAACTAATTTATA | | |
| | | | CTCCCACCAACAGTGTAAAAGC | | |
| | | | ATTCCTATTTCTCCACATCCTCTCA | | |
| | | | GCATCTGTTGTTTCTTGACTTTTTA | | |
| | | | ATGATTAGCATTCTAACTGG | | |
| | | | CGTGAGATGGTATTTCATTGTGGTT | | |
| | | | TTGATTTGCATTTCTCTAATGACCA | | |
| | | | GTGATGATGAGTTTTTTTTC | | |
| | | | ATATATTTGTTGGCCGCATAAATGT | | |
| | | | CTTCTTTTGAGAAGTGTCTGTTCGT | | |
| | | | ATCCTTCACCCACTTTTTGA | | |
| | | | TGGGGTTGTGTTTTTCTTGTAAATT | | |
| | | | TATTTAAGTCCCTTGTAGATTCTGG | | |
| | | | ATATTTTCCCTTTGTCAGAT | | |
| | | | GGATAGATTGCAAAAATTTTCTCC | | |
| | | | CGTTCTGTAGGTTGCCCGATCACTC | | |
| | | | TGATGATAGTTTCTTTTGCTG | | |
| | | | TGTAGAAGCTCTTTAGTTTAATCAG | | |
| | | | GTTCCATTTGTCAGTTTTGGCTTTT | | |
| | | | GTTGCAATTGCTTTTGGTGT | | |
| | | | TTTAGTCTTAAATTCTTTGCCCATG | | |
| | | | CCTATGTCCTGAATGGTATTGCCTA | | |
| | | | GATATTCTTCTAGGGTTTTT | | |
| | | | TTTTTGGCTTTAGGTCTTGCAGTTA | | |
| | | | AGTCTTTAATCTATCTTGAGTTAAT | | |
| | | | TTTTGTATAAGATATAAGAA | | |
| | | | AGGGGTCCAGTTTCAGTTTTCTGCA | | |
| | | | TATGGCTAGCCAGTTTTCCCAACA | | |
| | | | CTATTTATTAAATAGGGAATC | | |
| | | | TTTTCCCCATTGCTTGTTTTTGTCA | | |
| | | | GGTTTATCAAAGATCAGATGGTTG | | |
| | | | TAAATGTGTGGTGTTATTTCT | | |
| | | | GAGGCCTCTGTTTTGTTCCATTGGT | | |
| | | | CTATATGTCTGTTTTTGTTCAGTAC | | |
| | | | CATGCTGTTTTGTTTACTAT | | |
| | | | AGCCTTGTAGTATAGTTTGAACTC | | |
| | | | AGGTAGTGTGATGCCTCCAGCTTT | | |
| | | | GTTCTTTTTGCTTAGGATTGTC | | |
| | | | TTGGCAATACAGGTTCTTTTTTGGT | | |
| | | | TCCATATGAAATTTAAAGTAGTTTT | | |
| | | | TTCTAATTCTGTGAAGAAAG | | |
| | | | ACAATGGTAGCTTGATGGAAATAG | | |
| | | | CATTGAATCTATAAATTACTCTCAG | | |
| | | | CAATATGGCCATTTTCAGGAT | | |
| | | | ATTGATTCTTCCTATCTATGAGCAT | | |
| | | | GGAATGTTTTTCCATTTGTTTGTGT | | |
| | | | CCTCTCTGGTATCCTTGAGC | | |
| | | | AGTGGTTTGTAGTTCTCATTGAAGT | | |
| | | | AGTCCTTCACATCCCTTGTAAGTTG | | |
| | | | TATTCCTAGGTATTTTATTC | | |
| | | | TCTTTGCAGCAATTGTGAATGGGA | | |
| | | | GTTCACTCATGATTTGGTTCTCTGT | | |
| | | | TTGTCCTATATACATATGTTG | | |
| | | | GTATATAGGAATGCTTTTATTTTAA | | |
| | | | AGATGGAAGATGATGTCTCTCTAT | | |
| | | | GTAACTCAGGCAGGTCTCAAA | | |
| | | | CTCCTGGGCTCAAATGATCCTCCT | | |
| | | | ACCTTAACCTCCTGAGTAGCTGAG | | |
| | | | ACTTTAGTCACACACCACCATG | | |
| | | | CCTGACCAGGAATTGTTTTTCAACT | | |
| | | | TCATAGTGGTAAACAAAACATATG | | |
| | | | TGTTTTCAGTTCTCATGGAAC | | |
| | | | AAGCAGCTTAGTAGGAGAAACATA | | |
| | | | TGTTGAACTTCTAACCAGAGAAGT | | |
| | | | AAATCTATAATGACAAATCATA | | |
| | | | ATTTCTGAAGGGTATTAATTAGAT | | |
| | | | GTTTGAGTGAGGGGAAATATTGGA | | |
| | | | AGGTGCTCATAACTTTATAAAT | | |
| | | | GTTCTAAAATATTTCATGCTAATCA | | |
| | | | CATTAAAATTATATCAAAGTATAT | | |
| | | | AAACATATCATGGAAAACATA | | |
| | | | ATCAGCACCATGTACTCAACACCT | | |
| | | | AGGTTAAAAAATAGCATTAAAAAT | | |
| | | | TCTCTTTCCAGCTCACATTCTG | | |
| | | | CTCCCTCCCCAAATCCACAGATAA | | |
| | | | CCATCGAATTATATTTTGTTTTCTT | | |
| | | | CATTCCCTTACTTTCTTTAAG | | |
| | | | TTTTACACCCATGTATGTACCCATA | | |
| | | | AAAATCTATTAGCTAATTTTGGTTG | | |
| | | | TGCATGAATATTGTATCAAT | | |
| | | | GCAATTATACTGTATATATTCTGCT | | |
| | | | TTTGCACATATTTTTAGATTCATCC | | |
| | | | ATTTGTGGCATGTAGCTTTC | | |
| | | | CATTCATTTTCACTGCTGCTCAGTA | | |
| | | | TTGTATTACAAATTTTACATTTGTT | | |
| | | | TTAGGGAAGAGTCATAAACC | | |
| | | | ATCTTTAAGTTCTCCTATGTTACAA | | |
| | | | GTAATTTTGTAAATGATGTGACGT | | |
| | | | GGTGATTCTATTTCATTTTTT | | |
| | | | CCCATATAGATAATTTATATTATTA | | |
| | | | ATAATTCCTTCTATTTCATAAGCCA | | |
| | | | CGTTTCTATATATCTATATA | | |
| | | | AATATAGATATGTAGATATATGAA | | |
| | | | AGCAATATATATATGGATGTCTTTC | | |
| | | | TGGGCTATCTGTACTTTCACA | | |
| | | | CTGGCTAATTTGCTTGTTTTTTCAT | | |
| | | | CAATACTTCACTTCCTTAATTACTA | | |
| | | | CAACATAGCAGGGCCTGGCA | | |
| | | | TCTGCTAGATTAAATCTCTCAGCTT | | |
| | | | CTTTTTATTAAGATTGCCCTGAATT | | |
| | | | GTCCTGGTTATCCTGGGCCC | | |
| | | | CCTACTTTTTTTATATTTTTGAATA | | |
| | | | CATCTAAATAAATTTAGAATAAAT | | |
| | | | CTATTGTGTTCCATAAAACCC | | |
| | | | CTGTTGGGATTTCAATTGAACTGC | | |
| | | | AATTAAATTTTAGATCAGTTTTGGA | | |
| | | | AGAATTGACTCAATAGTGAGC | | |
| | | | CTTCCTACCCAAGACCATGGCATT | | |
| | | | TATTTTCATTTATTTATGATTTCTTT | | |
| | | | AATGCTTCTCAAAATTTTTT | | |
| | | | ATTTTCTCTATTATGGAAACGCACA | | |
| | | | TTTATAGTTTGACAAATTCCTAAGT | | |
| | | | ACTTCTAATTTTATTGTCAT | | |
| | | | TCCACATTATCTTTTTTGTTGTTGTT | | |
| | | | TTAAAAGACAGGGTCTCCCTCTGT | | |
| | | | CACCCAGGCTGGAGTGTACT | | |
| | | | GATGTGATTATAGCTCACTGCAGT | | |
| | | | CTCAACCTCCTGGGCTCAAGTGAT | | |
| | | | CCTCCCACGTCAGCCTGTGGAG | | |
| | | | TAGCTAGGACTACAGGCATGTGCC | | |
| | | | ACAATGCCTGGCTCATTTTTAAGTG | | |
| | | | TTAAGTTAAAAAAAGTTGTAG | | |
| | | | AAACAGTGTTTTGCTACATTTCCCA | | |
| | | | CGCTGGTCTCAAACTCCTGGCCCC | | |
| | | | AAGCAATCTTCCTGCCTCAGC | | |
| | | | TTCCCATATTCGGATTATACGCATG | | |
| | | | AGGCATTGCACCAGCCCCATGTGT | | |
| | | | TATCTTTTATAAAATTTAACA | | |
| | | | TTTAACTGATAATTGATACTGTATA | | |
| | | | TACATGAATTCAATTGGTATCTATT | | |
| | | | TTTAATATGGGAAATTTTAT | | |
| | | | GCAAATGAGCACATTTTTCTCCCTT | | |
| | | | CCTTCCTTCCTTCTTTCCTTGTTCTC | | |
| | | | TTTCTTTCTCTCTCTTTCT | | |
| | | | CTTTCTCTCTTTCTTTCTTTCTTTCT | | |
| | | | TTCTCACAGGGTGTCACTCTGTTGC | | |
| | | | CCAGGCGGAGTGCAGTGGC | | |
| | | | ACATGATCATAGCTCACTGCAACC | | |
| | | | TCCAACTCAAACACTTGAGTGATC | | |
| | | | CTCTGTCCCCCGTTTCCCAAGC | | |
| | | | AGCTGGGACTACAGGCACATGCCA | | |
| | | | CGATGCCAAGCTAATTTTTAAAAA | | |
| | | | TAATTTTTTTTGTAGATTCAGA | | |
| | | | GTCTTGCTATGTTGCCCAGGCTAAT | | |
| | | | CTCAAACTCCTGGCCTCAAGCAGT | | |
| | | | CCTCCCTCCTCAGCCTCCCAT | | |
| | | | TACAGGCATAAGCTGCCACTCCTG | | |
| | | | GACCTCTTTTTTTTTTTTTTTTTTTT | | |
| | | | TTTTTTGAGGCAGTCTCTCT | | |
| | | | CTGTCACCCAGGCTGGAGTATAGT | | |
| | | | GGCACGATCTCAGCTCACTGCGGG | | |
| | | | TTCAAGCAATTTTCATGCCTCA | | |
| | | | GCCTCCCAAGTAGCTGGGATTACA | | |
| | | | GGCATGGGCCACTATGCCCAGCTA | | |
| | | | ATTTTTGTATTTTTCATAGAGA | | |
| | | | CAGGATTTCACCATGTTGGCTAGG | | |
| | | | CTGGTATCAAACTCCTGACTTCAG | | |
| | | | GTGATCCGCCCACTTTGACCTT | | |
| | | | CCAAAATGCTGGGATTACGTGTGA | | |
| | | | GCCACCAAACCCAGCCCCTCATTT | | |
| | | | TCTTTTTGATTTTTATTTATTT | | |
| | | | TCCTCTGTTTTTCTTCTTTTGGATTT | | |
| | | | AGGGATGTGTGTGTGGAGGTGTAT | | |
| | | | TGAGTCCGTTTTTTCTTTCT | | |
| | | | ATTTGTGTGGAAATTATACACTTAT | | |
| | | | TCTTTGTTATTTTAGCAATTACTCT | | |
| | | | GGCTATTTTAACATGCAAAT | | |
| | | | ATAATGAAGTTTAGAATTAGCCAT | | |
| | | | TTTTTATAACTCTCCTTCTGACTAG | | |
| | | | TTGAAGAAATGAGAATGCTTT | | |
| | | | AACATCAAACAGCCAACTCTTTAC | | |
| | | | TTATACACTATTGCTATTCATTATA | | |
| | | | GCATTTTTAGTCTAGCTTCCT | | |
| | | | CCCTCCTCTTTCTCTCTCTCTCTCTC | | |
| | | | TGTCTCTCTCTCTCTCACTAATGTT | | |
| | | | TGCTATTTCTCCCTACAAT | | |
| | | | TCAGAATTTTATTTATGGATGAAGT | | |
| | | | ACATATATAATTTATTACAATTCAT | | |
| | | | TTTAATGAAAAACTTTTAGT | | |
| | | | GGTAAATTGTATTAGTCTTTGGGA | | |
| | | | AAAAACATTTATTGATACCATTTTC | | |
| | | | TCATTACTTAAAAATAGTTTC | | |
| | | | ACTTCATATAGAATTCTATGTCGAC | | |
| | | | AGTAATTTTCTTTCACGAAGTAGA | | |
| | | | AAATATTAAGTTACAGTATTT | | |
| | | | TGGCTTCCATTACTGCTGTTAAGCA | | |
| | | | TTCAGATCATCAGAGAAATGCAAA | | |
| | | | TCAGAACCACAATGAGATGCC | | |
| | | | ATCTCATGCCAGTCAGAATGGCAA | | |
| | | | TCATTAAAAAGTCAGGAAACAATA | | |
| | | | GATGCTGGTGAGGCTGTGGAGA | | |
| | | | AATAAGAATGCTTTTACACTGTTA | | |
| | | | GTGGGAATGTAAATTAGTTCAACC | | |
| | | | ATTGCTCTTAAGGGCTCTTTGT | | |
| | | | CTTTAATGATCCGCATTTTTATTAT | | |
| | | | GATGTGTCTAGGCAGTTATTATTGT | | |
| | | | GTTTATTCTATTTCTTTATT | | |
| | | | TGCTGTCTATCCAAGATTTGAGGA | | |
| | | | TTAATTTTTTAATTTCTAGAAAATT | | |
| | | | CACAAGTATTATTTATTTATT | | |
| | | | CAATTATTACCTCTTTCTATTATTT | | |
| | | | CCTTTTTAAAATAAAAGGGTATAT | | |
| | | | GTTAGAATTTTTCACTCTCTC | | |
| | | | CTTTATGCCTTTAACTTCATATTTT | | |
| | | | CTATTTCTTTGAATTTCTGGGCTGC | | |
| | | | ATTCTTAAGAATTCTAAAAC | | |
| | | | ATATATTTTAGTTTCTAAAAGTTTC | | |
| | | | ATTAGATTTCTGTTCAAAATTCCTT | | |
| | | | CCATTTGTGATCTTTCGAAT | | |
| | | | GTGCTTCTGCTTTAGGCATTAGTAG | | |
| | | | TGGACATTCTGGTTCCCCATTGAGC | | |
| | | | TTCCCTGCATCAGCTGTTTT | | |
| | | | GCCTGGTGGCTGCCACCAACGCTT | | |
| | | | TTAGCTACCTCCCTCCTCAAACTTT | | |
| | | | GGGGTCAGGCCACACACTATA | | |
| | | | AAGGATTGGAAAAAAAAATGAAA | | |
| | | | ATATGAAAAACTTACACTTTGTAT | | |
| | | | CAGTCAGGAGAAGGATAATCTTC | | |
| | | | ACACTACAGTTTATGCTTCAGAAG | | |
| | | | CCACCCCTTCTCTGTGGATTAGACC | | |
| | | | ATGACTAGAAGTTTCCTGAGA | | |
| | | | CCATCCCTTGCCCAGCTCTTTTGGT | | |
| | | | GATCCCCTTCACTTCCTCTGTTACA | | |
| | | | GGTTTCCCTGATGAGCACTC | | |
| | | | CTTCAATAAAACATAGTCATCCAA | | |
| | | | ATCCCAATCTCAAGCACGGTGTCA | | |
| | | | CGGGAACCTGATCTAAGTCAGC | | |
| | | | ATTTTCTTTATTCTTAATCACAACT | | |
| | | | AGTTGATAGTCCATATCTAATATAT | | |
| | | | AATGAATGTACAGTTGTTTC | | |
| | | | TGTTGGAGTTCACATATGATGCCTT | | |
| | | | GTTTCCTTTGTAGTTTTGTGATTGA | | |
| | | | TAGCTTCGAACTGCTCATTT | | |
| | | | ACCTTGACCTTTTGAATTCTTTGAA | | |
| | | | AACTGAGTTAAGTCTGATTTTCCA | | |
| | | | GAGTTTTTATGTTTGCTTCTG | | |
| | | | TCAGTTGCAGAGAATCAATGAGAA | | |
| | | | GAACACTTTAAATTCTTGTTTTCGG | | |
| | | | TTTTTTTCCAATCACATAAGT | | |
| | | | AGGATTTACCTGAATATATATATA | | |
| | | | ATATATAAACATATATTTATATAA | | |
| | | | AATAAAAACATATAAAATATGA | | |
| | | | AATATATAATACAGTATAAAATCT | | |
| | | | ATTTTATGTAAAATCTATTTTATGT | | |
| | | | AAACATCATAATTAAATATAT | | |
| | | | ATTTAAATAATATAAATATAATAA | | |
| | | | ATATTTGAAGCAATTGTATTTTTTA | | |
| | | | AAAATTTCTTCTAAAGAAAAC | | |
| | | | CAGGATACATGTGCAGAACCTGCA | | |
| | | | GGTTTGTTACATAGGTATACGTGT | | |
| | | | GCCATGGTGGTTTGCTGCACCT | | |
| | | | ATTGACCCGTCCTCTAAGTTCCCTC | | |
| | | | CCCTCACCTCCCACCCCCCAGCAG | | |
| | | | CCCCTGGTGTGTGTTGTTCCC | | |
| | | | CTCTCTGTGTCCATGTATTCTCGCC | | |
| | | | TCCCACTTATGAGTGAGAACACGC | | |
| | | | GATGTTTGCTTTTCTGTTCCT | | |
| | | | GTGTTAATTTGCTGAGGATGATAG | | |
| | | | CTTCCAGCTTCATCCACGTCCCTGC | | |
| | | | AAAGGACATGATCTCATTCCT | | |
| | | | CAATACTATGGCTACGTAGTATTC | | |
| | | | CATGGTGTATATATACCACATTTTC | | |
| | | | TTCATCCAGTCATGCAAATTT | | |
| | | | ATATGAATGTCAATTCTTTTATAGT | | |
| | | | GATCTTCTGGGGCTATTACAATAT | | |
| | | | ATAGGGCTGTTTTTTTAAAAC | | |
| | | | TAATTATATTTATTTCATGTTGCTT | | |
| | | | TAACTTATTAAAAAACAGACTGAA | | |
| | | | GAAAGACTGGGTGTGAAGTCA | | |
| | | | GTAAATTAATTTCAAATTAAATAA | | |
| | | | ACTTTTCTACAGCTATTTTATGCTC | | |
| | | | AATAACTTTCTACTTATTCTT | | |
| | | | GAGTTCAAAACTATATGGGTTCAC | | |
| | | | ATTTAAATTATATAGTGTATTTTCT | | |
| | | | CCATAAACTGAAGTTGTTAGA | | |
| | | | ACATTGATTTTTTTAAGTAAATGGA | | |
| | | | TTTTTGCACCACTTCAAGAAAGAA | | |
| | | | ACCTTCAAACAGCCTGGAAAT | | |
| | | | ATCACATCAATAAAGCACAACCTG | | |
| | | | GGAATCAAAGTATTAGGGTACCTT | | |
| | | | GTTACTGAGATTATGGATGTGA | | |
| | | | TGCTTCTGTGGGCCATTAGCATGTG | | |
| | | | CACTGTGTGTATGATATGCTCTATG | | |
| | | | TTCTCTTCCCACTAATAATT | | |
| | | | TTATTTTTAATTTCAGCAAGATTTA | | |
| | | | GTCTCAAATAACACAATAATAATG | | |
| | | | GAGGTCATTGTGAAGTAGTGG | | |
| | | | ATGTAAATAGATCTGATGTGGTTTT | | |
| | | | GGTTTATTGCAGTAATTGTTTTGAC | | |
| | | | TAATTCTCTAGTTTTTCAAC | | |
| | | | TTTTGATTGTTTAAGATGGTTCTTG | | |
| | | | AGTCCTTTTGACATGACCCTATCTA | | |
| | | | TTTTTGATAACTTCATAGCC | | |
| | | | TTTAGTATAAAAACAGGTAGGCTT | | |
| | | | ATATTACATATTTCCAACTTCAAAC | | |
| | | | TTGTTATTTATTTATCTAAGA | | |
| | | | CTATACAGTTCTTTTCAGAGAAAA | | |
| | | | ACCTTCTTTATAAACCAGAATCTTA | | |
| | | | ACAGGAAGAGTGCTCATTTTA | | |
| | | | ATTGAGCTGATCATGTTTCTAGGAT | | |
| | | | TTTTTAGTTAAAAGAAAATACATA | | |
| | | | TTTTAAAAATATAAATTATAT | | |
| | | | TTTTATTTCATAGTGGTATTTTCAA | | |
| | | | TTTTGTCTGGGATAATAAGATGTTT | | |
| | | | TATTTAACTTGTTTGATTTT | | |
| | | | GTAGTTTTATCTTTGTGGGAAGGA | | |
| | | | CCTGGTAAGAGGTAATTGAATCAT | | |
| | | | GGGGCCAGGACTTTCCCATGAT | | |
| | | | GTTCTCATGATAATGAATAAGTTTC | | |
| | | | ATGAGATCTGTTGGTTTCATAATGT | | |
| | | | GGAGTTTCCCTGCAAAGGCT | | |
| | | | CTTGTCCTGTCTGTGCCATTTGAGA | | |
| | | | CATGCCTTTCAACTTCTGCCCTGAT | | |
| | | | TGTGAGGCCTCCCCCGCCAT | | |
| | | | GTGGAATTGGGTCTTACTTTTGTAA | | |
| | | | ATTGCCCAGTCTCAGGTATGTCTTT | | |
| | | | ATCAGCAGCCTGAAAACTGA | | |
| | | | CTAATATAGTAAGTTGGCACCAGT | | |
| | | | AGAGAGGGGCACTGCTGAAAAGG | | |
| | | | TACCCGAATATGTGGAAGCAACT | | |
| | | | TTAAACTGGGTAACAGGCAGAGGT | | |
| | | | TGGAATGGTTTGGAGGGCTCATAA | | |
| | | | GAAGACAGGAAAGTGTGGGAAA | | |
| | | | TTTGGAACTCCCTAGAGACTTGTTG | | |
| | | | AATGGCTTTAACCAAAATGCTGAT | | |
| | | | AATAATATGAACAATGAAGTC | | |
| | | | CAGGCTGAGGTGGTCTCAGACAAA | | |
| | | | GATAAGGAACTTCTTGGGAACTGG | | |
| | | | AGCAAAGGTGACTCTTGTTATG | | |
| | | | TTTTAGACATAAAGCAAAGAGACT | | |
| | | | GGAGGCATTTTGCCCCTGCCCTAG | | |
| | | | AGATTTGTGGGACATTAAACTT | | |
| | | | GAGACAGATTATTTAGGGTATCTG | | |
| | | | GAGGAAGAAATTTTTATGCAGCAA | | |
| | | | AGCATTCAAGAGGTGACTTGGT | | |
| | | | TGCTATTAAAGGCATTCAGTTTTAA | | |
| | | | AAGGGAAATACAGCATAAAAGTTC | | |
| | | | AGAAAATTTTCAGCCTGACAA | | |
| | | | TGCAGTAGAAAAGGAAAACCAATT | | |
| | | | TTCTGAGGAGAAATTTAAGCTGGC | | |
| | | | TGCAGACATTTACATAAGTAAC | | |
| | | | AAGAAGCTGAATGTTAATCACTAA | | |
| | | | GACAATGAGGAAAATGTCTCCAGG | | |
| | | | GCATGTCAGAGACCTTTGTGGC | | |
| | | | AGCCCCTCCCATCACAGACCAGGA | | |
| | | | CCTTTAGAAGGAAAAATGGCTTCG | | |
| | | | TGGGCTGGTCACAGGGTCCCTC | | |
| | | | TGCTGTGTGCAGTCTAGGGACTTG | | |
| | | | GTGCCCTGTGTCCCAGCAGCTCCA | | |
| | | | TCCATGACTAAAAGGGGCCAAG | | |
| | | | GTACAGCTTGGGCTGTGGCTTCAG | | |
| | | | AGGGTGGAAGCCCCAAGTCTTGGC | | |
| | | | AGCTTCCATATGGTGTTGAGCC | | |
| | | | TGGGTTCACAGAAGTCAAGAACTG | | |
| | | | AGGTTTGGGAACTTACACCAAGAT | | |
| | | | TTCAGAGGATGTATGGAAATGC | | |
| | | | CTGGATGCCCAGGCAGAAGTTTGC | | |
| | | | TGCAGGGGCAAGGCCCTCATGGAG | | |
| | | | AACCTCTGCTAGGGCAGTGAAG | | |
| | | | AAGGGAAAAGTATGGTGGGAGCC | | |
| | | | CCCATACAGAGTCCCTACTGAGGC | | |
| | | | ACCACCTAGTGGAGCTTTGAGAA | | |
| | | | GAGGGCCACTGTCCTCCAGAACTC | | |
| | | | AGGATGGTAAATCCACCACGCACC | | |
| | | | TGGAAAAGCTGCACACAATTCC | | |
| | | | AGCCTGTTAAAGCAGCCAGGAGGG | | |
| | | | GGCTATACCCTGCAAAGCCACAGG | | |
| | | | GGCGGACCTGCTCAAGGCTGTG | | |
| | | | GGAGACCACCTCTTGCATCAGTGT | | |
| | | | GACCTGGATGTGAGACATGGAGTC | | |
| | | | AAAGGAGATCATTTTGGAGCTT | | |
| | | | TAAGATTTGACTGCCCCACTGGAT | | |
| | | | TTCAGACTTTCATGGGGCCTGTAG | | |
| | | | CCCCTTCGTTTTGGCCAATGCC | | |
| | | | TCCCATTTGGAGTGGCTGTATTTAC | | |
| | | | CCAATGCCTGTATCCCCATTGTATC | | |
| | | | TAGGAAGTAACTAACTTGCT | | |
| | | | TTTGATTTTACAGGCCCATAGGTG | | |
| | | | GAAGGGCGATGTTTCTTTCTGGAG | | |
| | | | GCTCCAGGGAGAACTCTGTTTT | | |
| | | | CTTACCTTTTCTGGATTCTAGAGGC | | |
| | | | TTCCCACAATCCTTGGCTTAAGGTC | | |
| | | | CATCTTTAAGCTTTGTCTCT | | |
| | | | GATGAGACTTTGGACTGCGGACTT | | |
| | | | TTGAGTTAATGCTGAAATGAGTTA | | |
| | | | AGACTTTGGGTGACTGTTGCGA | | |
| | | | AGACATGATTGGTTTTGAAATGTG | | |
| | | | AGAACATTTAAGAGGGGCCAGGG | | |
| | | | GCAGAATGATATGGTTTGACTTT | | |
| | | | GTCCGCAGTCAAATCTCATCTTGA | | |
| | | | ATTTCTATGTGTTTGGAGAGGTACC | | |
| | | | CGGTGGGAGGTAATTGAATCA | | |
| | | | TGAGGGCAGGTCTTTTCTGTGCTGT | | |
| | | | TCTCATGATGGTGAGTAAGTCTCA | | |
| | | | TGAGATCTGATGGTTTTATAA | | |
| | | | AGGGGAGTTTCCCTCCCCAAGTTC | | |
| | | | TTCTCTTGTCTGCCATCATGTGCGA | | |
| | | | TGTGCCTTTCACCTCTGCCAT | | |
| | | | GATTATGAGGCCTCCCTGGCCATG | | |
| | | | TGGAACTGTGAGTCCATTAAACCT | | |
| | | | CTTTCTTTTGTAAATTGCCCAA | | |
| | | | TCTTGGGAATGTCTTTATCAGCAGT | | |
| | | | GGGAAAACGGATTAATATACTAAT | | |
| | | | TTATAGCTAGTAGGTAAAAAG | | |
| | | | CCAGGGACTTGCCATTAGCGTTGG | | |
| | | | AAGTGGGGTTGTGGGGGCAGTCTT | | |
| | | | GTGGAACTGAGCCCTTAACCTG | | |
| | | | TGGGGTTGAATGATATCTCCAGGT | | |
| | | | ATATCATGTCAGAATTGAATTCAA | | |
| | | | TTAGAGGATACCTAGCTTGCCT | | |
| | | | TCAATGCAGAATTGCTTGCTGGTG | | |
| | | | AGGAGAAATCCCTATACACATTTT | | |
| | | | GGTGACCAGAGGTAAAGCATTTT | | |
| | | | TATGTTGATTCTTGAGTGAGAGAG | | |
| | | | TAGAAATAACACTGGTTTTTTCCCT | | |
| | | | ATGTCCTTACAACCACCAATT | | |
| | | | GGATACATTGTTTCAGTATTTTGAA | | |
| | | | ATTTTTCATTTAATTTTTATAAATT | | |
| | | | TTCTTTTTAAATTTTAGATT | | |
| | | | CTACAATATCTCCAATTCTTCAGTT | | |
| | | | TATTCCCTCTTACTATGTATAAGTA | | |
| | | | TTTCCCCAAGTTTCACTTTA | | |
| | | | TCTTTCTATTACTTTTTTTACATAAT | | |
| | | | AGAGCTATAAAGGCAATTCACAAT | | |
| | | | TCTCTCTTTTCTCATATATA | | |
| | | | ATATAGAGCATATTATAAATACTC | | |
| | | | TACTTTGGAAAATTATTCTTTATAG | | |
| | | | GAAATTACAGATAATATTTGA | | |
| | | | TGAAGAAAATCGAATATAATCATT | | |
| | | | TTTCAATACTTAGGATAACAGATT | | |
| | | | CAGGCAAAGATAAAACATTAAA | | |
| | | | GGAAAAGTTAGTGAAAACTATTAA | | |
| | | | TATATAGTGGAGGCATCACGTTGT | | |
| | | | TATGAACTTCATTGATCAATAC | | |
| | | | TGATACCACTAAAAATGGAACAAC | | |
| | | | ATGTAATTATGTGCTCAATGTGAT | | |
| | | | GAATATGAAGTAGACTGCACCA | | |
| | | | CTCTGCAGTACAGTCAGGAAATAA | | |
| | | | GAAACCAAGTCCAATCAAAATAGC | | |
| | | | CCTAAAGCTACCTTCCAGTTTA | | |
| | | | TAAAAAGTATGAAGAATAGAGGG | | |
| | | | CCAATTAAATCATACCATAAAGAG | | |
| | | | TCAAATACAGGGCATGCAACATA | | |
| | | | GCTGCTGATTGGATTTATTCAACAT | | |
| | | | GTCAGTGGCATGAATACAATAGGA | | |
| | | | GGCAGGTAGGGAGAAGGCACT | | |
| | | | ACCCTGAATTATGAGACTGAAGAG | | |
| | | | ATATAATAAACAAATGCAATGTGT | | |
| | | | GGACTTGGTTGGGATCTTCATT | | |
| | | | CAAAGACCAACTATAAAAAGACAT | | |
| | | | TGTTGTGAGAATTGAGGAAATTTG | | |
| | | | AATGAGAAATGTATTTTTATCT | | |
| | | | AATTTGTTAGCTGTGATAATAGTAT | | |
| | | | TGTGGGAGTAAGAAGCTATTCATA | | |
| | | | TTTCTATATATATATACCAAG | | |
| | | | TACATAGGAGTGAAATAATACAAA | | |
| | | | ATCTGGAATTTGCCTTAAAATTCCT | | |
| | | | CTGCAAAATTATAAAAAAGAA | | |
| | | | CGATGACAAACTAAAAAGGTGTAG | | |
| | | | TATTCTTCTATGGCTGCTATAACAA | | |
| | | | ATGACCAAAAAACATAGTGAC | | |
| | | | TGAAAATAACCCACATTTATTATCT | | |
| | | | TACAGTTCCATAGGTTAGAAGTTC | | |
| | | | AACATGGGTCTCATGAGATCA | | |
| | | | AAAGCAAGGCCTTGGCAGGGTGAC | | |
| | | | GTTTCTTTCTGGAGGTTCCAGGGG | | |
| | | | GAACTCTGTTTTCTTACTTTTT | | |
| | | | TTAGATTCTACAGGCTTCCCACAA | | |
| | | | TCCTTGGCTTAAGGTCCATCTTTAA | | |
| | | | AGACAGCAACGTTTCATCTCT | | |
| | | | CTACCTATTCTTTCATCCTTACATC | | |
| | | | TTTCTCTAACTATTCCTTTTCTTCTG | | |
| | | | TCTTCCACTTTTAAGAGCC | | |
| | | | TTTTTGAGTCTATTGAGGCCAACTG | | |
| | | | GACAATCAAGGATTATCTCCCTAT | | |
| | | | GTTAAGGTCAATTGATTAGTG | | |
| | | | ACCTAATTCCATCTACAATCACAA | | |
| | | | TTCCTCTTTGCCATATAATGTAAAA | | |
| | | | TATTCATACCTCTAAGGATTA | | |
| | | | GGACATGGACATCTTTGAGGGTCA | | |
| | | | TTAGTCATCTTACCACAGGAAGGA | | |
| | | | AGGAAGGAAGGAAGGAAGGAAG | | |
| | | | GAAGGAAGGAAGGAAGGAAGGAA | | |
| | | | AGGGAGGAGAGGAGAGGAGAGGT | | |
| | | | AGGACGGAAGAAGAAAAAAATAG | | |
| | | | T | | |
| | | | ATGAAAAAATCTTGATAAATTTGA | | |
| | | | AAACTGGGTGAATAATATGTGGAA | | |
| | | | TTCTCTCTATTTTTGTTAATGT | | |
| | | | TGGAAAATTTAATAAAAACAATGA | | |
| | | | ACAGTGA (SEQ ID NO: 27) | | |
| | | NM_001032998.2 | ACATTTTCAAGGAATTCTTGAGAG | NP_001028170.1 | MEPSSLELPADTVQRIA |
| | | | GTTCTTGGAGAGATTCTGGGAGCC | | AELKCHPTDERVALHL |
| | | | AAACACTCCATTGGGATCCTAG | | DEEDKLRHFRE |
| | | | CTGTTTTAGAGAACAACTTGTAAT | | CFYIPKIQDLPPVDLSLV |
| | | | GGAGCCTTCATCTCTTGAGCTGCC | | NKDENAIYFLGNSLGLQ |
| | | | GGCTGACACAGTGCAGCGCATT | | PKMVKTYLEEELDKWA |
| | | | GCGGCTGAACTCAAATGCCACCCA | | KIAAYGH |
| | | | ACGGATGAGAGGGTGGCTCTCCAC | | EVGKRPWITGDESIVGL |
| | | | CTAGATGAGGAAGATAAGCTGA | | MKDIVGANEKEIALMN |
| | | | GGCACTTCAGGGAGTGCTTTTATA | | ALTVNLHLLMLSFFKPT |
| | | | TTCCCAAAATACAGGATCTGCCTC | | PKRYKILL |
| | | | CAGTTGATTTATCATTAGTGAA | | EAKAFPSDHYAIESQLQ |
| | | | TAAAGATGAAAATGCCATCTATTT | | LHGLNIEESMRMIKPRE |
| | | | CTTGGGAAATTCTCTTGGCCTTCAA | | GEETLRIEDILEVIEKEG |
| | | | CCAAAAATGGTTAAAACATAT | | DSIAVI |
| | | | CTTGAAGAAGAACTAGATAAGTGG | | LFSGVHFYTGQHFNIPAI |
| | | | GCCAAAATAGCAGCCTATGGTCAT | | TKAGQAKGCYVGFDLA |
| | | | GAAGTGGGGAAGCGTCCTTGGA | | HAVGNVELYLHDWGV |
| | | | TTACAGGAGATGAGAGTATTGTAG | | DFACWCSYK |
| | | | GCCTTATGAAGGACATTGTAGGAG | | YLNAGAGGIAGAFTHEK |
| | | | CCAATGAGAAAGAAATAGCCCT | | HAHTIKPARSEFFN (SEQ |
| | | | AATGAATGCTTTGACTGTAAATTT | | ID NO: 30) |
| | | | ACATCTTCTAATGTTATCATTTTTT | | |
| | | | AAGCCTACGCCAAAACGATAT | | |
| | | | AAAATTCTTCTAGAAGCCAAAGCC | | |
| | | | TTCCCTTCTGATCATTATGCTATTG | | |
| | | | AGTCACAACTACAACTTCACG | | |
| | | | GACTTAACATTGAAGAAAGTATGC | | |
| | | | GGATGATAAAGCCAAGAGAGGGG | | |
| | | | GAAGAAACCTTAAGAATAGAGGA | | |
| | | | TATCCTTGAAGTAATTGAGAAGGA | | |
| | | | AGGAGACTCAATTGCAGTGATCCT | | |
| | | | CTTCAGTGGGGTGCATTTTTAC | | |
| | | | ACTGGACAGCACTTTAATATTCCT | | |
| | | | CCCATCACAAAAGCTGGACAAGCG | | |
| | | | AAGGGTTGTTATGTTGGCTTTG | | |
| | | | ATCTAGCACATGCAGTTGGAAATG | | |
| | | | TTGAACTCTACTTACATGACTGGG | | |
| | | | GAGTTGATTTTGCCTGCTGGTG | | |
| | | | TTCCTACAAGTATTTAAATGCAGG | | |
| | | | AGCAGGAGGAATTGCTGGTGCCTT | | |
| | | | CATTCATGAAAAGCATGCCCAT | | |
| | | | ACGATTAAACCTGCGAGATCGGAG | | |
| | | | TTCTTTAATTAGGAATGGAATGCA | | |
| | | | ACAGATTTGGACAAGTCAAGGA | | |
| | | | CAAGAGCTTTAGAGAGACCAAAGA | | |
| | | | GTTTTTCACTGTTAAAGTGTCCAGT | | |
| | | | ATGTAGCCGAGAACCATATGG | | |
| | | | AGAACATCAAATACAGTGGAACAA | | |
| | | | ATGTAACTGCTATTGATGTCACACT | | |
| | | | TTGTGAAGTAGTCTTTGTTGC | | |
| | | | TTAAAAAGGGTGACATCTAGTGGC | | |
| | | | TAAACATGTTATTTCAAATAAATA | | |
| | | | ATATCGAAATAACATTTCTTCT | | |
| | | | CATGGTCCACTCATTCACTCTTTAA | | |
| | | | CAAGTATTTTGAAGTATATATGTTT | | |
| | | | GAATTATGTGTTCTTCTTTT | | |
| | | | TGACAATTTGACTATATGTTGATA | | |
| | | | GTGCAATAATTGTGCAGTTTAAGC | | |
| | | | CTTCAATAAAGAGGTAGAATGT | | |
| | | | GATGAAAATTGGAAGGAAACCTGA | | |
| | | | GGGGGCATTCTTAGTGCTTGGTTA | | |
| | | | AACAGAAAGCTTAACAGTTCAT | | |
| | | | GAAGGCTGGTCTAAGAAAGGAATT | | |
| | | | ATAAGCATGGGTGACCCACCTGGT | | |
| | | | CTAGAGAGTGTATCCCCAGATA | | |
| | | | TATAACATTGCATTTTAGAAGTCTA | | |
| | | | ATATTTGGTATATAATTTTTGAAAT | | |
| | | | AGTCCTTTATGTGATGTTTC | | |
| | | | CATTAGCAAACAGCAAATTGCATC | | |
| | | | TGTACCAAGAGATTTCACTTCCTTT | | |
| | | | TTTGTTTAAATATGCATTTTG | | |
| | | | GACATTGTTCAAAACCTATGACCT | | |
| | | | AAGGCTTTTCCAAGAGCCCTTTGC | | |
| | | | CCATAAAGAGAATGAATAAATT | | |
| | | | AGAGGCCAGAGTCAACGCACGGC | | |
| | | | ATTAA (SEQ ID NO: 29) | | |
| | | NM_001199241.2 | ACATTTTCAAGGAATTCTTGAGAG | NP_001186170.1 | MEPSSLELPADTVQRIA |
| | | | GTTCTTGGAGAGATTCTGGGAGCC | | AELKCHPTDERVALHIL |
| | | | AAACACTCCATTGGGATCCTAG | | DEEDKLRHFRE |
| | | | CTGGAATATAAAGAATGGCTTATC | | CFYIPKIQDLPPVDLSLV |
| | | | AGTGGAGACCATCGACAGTTGAGA | | NKDENAIYFLGNSLGLQ |
| | | | AAAGAAGAAGCCCAAAAAGTAC | | PKMVKTYLEEELDKWA |
| | | | AAGAATGAAAATCGAGAGTTTTTA | | KIAAYGHI |
| | | | GAGAACAACTTGTAATGGAGCCTT | | EVGKRPWITGDESIVGL |
| | | | CATCTCTTGAGCTGCCGGCTGA | | MKDIVGANEKEIALMN |
| | | | CACAGTGCAGCGCATTGCGGCTGA | | ALTVNLHLLMLSFFKPT |
| | | | ACTCAAATGCCACCCAACGGATGA | | PKRYKILL |
| | | | GAGGGTGGCTCTCCACCTAGAT | | EAKAFPSDHYAIESQLQ |
| | | | GAGGAAGATAAGCTGAGGCACTTC | | LHGLNIEESMRMIKPRE |
| | | | AGGGAGTGCTTTTATATTCCCAAA | | GEETLRIEDILEVIEKEG |
| | | | ATACAGGATCTGCCTCCAGTTG | | DSIAVI |
| | | | ATTTATCATTAGTGAATAAAGATG | | LFSGVHFYTGQHFNIPAI |
| | | | AAAATGCCATCTATTTCTTGGGAA | | TKAGQAKGCYVGFDLA |
| | | | ATTCTCTTGGCCTTCAACCAAA | | HAVGNVELYLHDWGV |
| | | | AATGGTTAAAACATATCTTGAAGA | | DFACWCSYK |
| | | | AGAACTAGATAAGTGGGCCAAAAT | | YLNAGAGGIAGAFIHEK |
| | | | AGCAGCCTATGGTCATGAAGTG | | HAHTIKPALVGWFGHE |
| | | | CGGAAGCGTCCTTGGATTACAGGA | | LSTRFKMDNKLQLIPGV |
| | | | GATGAGAGTATTGTAGGCCTTATG | | CGFRISNP |
| | | | AAGGACATTGTAGGAGCCAATG | | PILLVCSLHASLEIFKQA |
| | | | AGAAAGAAATAGCCCTAATGAATG | | TMKALRKKSVLLTGYL |
| | | | CTTTGACTGTAAATTTACATCTTCT | | EYLIKHNYGKDKAATK |
| | | | AATGTTATCATTTTTTAAGCC | | KPVVNIIT |
| | | | TACGCCAAAACGATATAAAATTCT | | PSHVEERGCQLTITFSVP |
| | | | TCTAGAAGCCAAAGCCTTCCCTTC | | NKDVFQELEKRGVVCD |
| | | | TGATCATTATGCTATTGAGTCA | | KRNPNGIRVAPVPLYNS |
| | | | CAACTACAACTTCACGGACTTAAC | | FHDVYKF |
| | | | ATTGAAGAAAGTATGCGGATGATA | | TNLLTSILDSAETKN |
| | | | AAGCCAAGAGAGGGGGAAGAAA | | (SEQ ID NO: 32) |
| | | | CCTTAAGAATAGAGGATATCCTTG | | |
| | | | AAGTAATTGAGAAGGAAGGAGAC | | |
| | | | TCAATTGCAGTGATCCTGTTCAG | | |
| | | | TGGGGTGCATTTTTACACTGGACA | | |
| | | | GCACTTTAATATTCCTGCCATCACA | | |
| | | | AAAGCTGGACAACCGAAGGGT | | |
| | | | TGTTATGTTGGCTTTGATCTAGCAC | | |
| | | | ATGCAGTTGGAAATGTTGAACTCT | | |
| | | | ACTTACATGACTGGGGAGTTG | | |
| | | | ATTTTGCCTGCTGGTGTTCCTACAA | | |
| | | | GTATTTAAATGCAGGAGCAGGAGG | | |
| | | | AATTGCTGGTGCCTTCATTCA | | |
| | | | TGAAAAGCATGCCCATACGATTAA | | |
| | | | ACCTGCATTAGTGGGATGGTTTGG | | |
| | | | CCATGAACTCAGCACCAGATTT | | |
| | | | AAGATGGATAACAAACTGCAGTTA | | |
| | | | ATCCCTGGGGTCTGTGGATTCCGA | | |
| | | | ATTTCAAATCCTCCCATTTTGT | | |
| | | | TGGTCTGTTCCTTGCATGCTAGTTT | | |
| | | | AGAGATCTTTAAGCAAGCGACAAT | | |
| | | | GAAGGCATTGCGGAAAAAATC | | |
| | | | TGTTTTGCTAACTGGCTATCTGGAA | | |
| | | | TACCTGATCAAGCATAACTATGGC | | |
| | | | AAAGATAAAGCAGCAACCAAG | | |
| | | | AAACCAGTTGTGAACATAATTACT | | |
| | | | CCGTCTCATGTAGAGGAGCGGGGG | | |
| | | | TGCCAGCTAACAATAACATTTT | | |
| | | | CTGTTCCAAACAAAGATGTTTTCC | | |
| | | | AAGAACTAGAAAAAAGAGGAGTG | | |
| | | | GTTTGTGACAAGCGGAATCCAAA | | |
| | | | TGGCATTCGAGTGGCTCCAGTTCCT | | |
| | | | CTCTATAATTCTTTCCATGATGTTT | | |
| | | | ATAAATTTACCAATCTGCTC | | |
| | | | ACTTCTATACTTGACTCTGCAGAA | | |
| | | | ACAAAAAATTAGCAGTGTTTTCTA | | |
| | | | GAACAACTTAAGCAAATTATAC | | |
| | | | TGAAAGCTGCTGTGGTTATTTCAGT | | |
| | | | ATTATTCGATTTTTAATTATTGAAA | | |
| | | | GTATGTCACCATTGACCACA | | |
| | | | TGTAACTAACAATAAATAATATAC | | |
| | | | CTTACAGAAAATCTGATATAATTTT | | |
| | | | TCAGAGTCTGTGGCACTAAGG | | |
| | | | AGTCCACAGGGCTGCCTAGGTGCT | | |
| | | | TTGTGTTTGGGGGACCAAAACTGT | | |
| | | | GTTGGTTCAACTATTATCTATA | | |
| | | | CAGTCTCTATAAGCTGTCACATTTC | | |
| | | | ATGGTCATTGAAATGTTTTATGTTG | | |
| | | | GTTTAATTTCTGATTTAACT | | |
| | | | CACAACTTCATAATGTATCTGCAA | | |
| | | | TTATTGTGTCAAATTTAGAAATATT | | |
| | | | ACTTTAGCTTCAATTTACCAA | | |
| | | | GGAGTTTCTTTGAAGCATTGTAGTC | | |
| | | | TGATATATATATATATATATATATA | | |
| | | | TATATATATATATATATATA | | |
| | | | TATATGTGTGTGTGTGTGTGTGTAT | | |
| | | | ATATATATATATATATCATATATAT | | |
| | | | ATGATAGTGGCTTTCAAATT | | |
| | | | TTTTTGGGTACAATCCACATTGCTC | | |
| | | | CTGCTGATCTGTAATATCAGAAAC | | |
| | | | CAGTATTTATGTGAATATATG | | |
| | | | AGAAATATTATTGATTCTAAGATA | | |
| | | | TTTTATCATATTTTAACATCTTTGA | | |
| | | | AAGAGGACCCATCTTTCAATT | | |
| | | | TTCGATCAATAGTTTCTTACAGTCA | | |
| | | | CCATTGGCCATCTTTCTCGTTACCA | | |
| | | | TCTATGAAATTAGCATGCAT | | |
| | | | CTCAAATAAACAGTTACCATCTTCT | | |
| | | | ATTTGATAAAATAGTCTAAATAGC | | |
| | | | AAAAATAAAAGTTTTTACAAT | | |
| | | | TATTTGCCTGTGCTCTAATAGGTAC | | |
| | | | TATTCTATTTTATCTCATAAGAAAT | | |
| | | | GTTGGAAACTCATTATATTG | | |
| | | | ATTTCCTTACCCACTCATGGGCCCT | | |
| | | | AATTCACACTTTTTAAGAATGTTTC | | |
| | | | TTTCTTTAATGTTATCATAA | | |
| | | | TCTCTTACTTTTTAAATCAGAACTT | | |
| | | | CCCCTAATATAAGAGCTTAGATAT | | |
| | | | TATATTACTATGTTTCCATAG | | |
| | | | TAAATAAATAACCCCAAGATCTTT | | |
| | | | TTGGGGATTAGAGATATAAGAAAT | | |
| | | | ATGTGCTCCATCTCTTGACATC | | |
| | | | TTTATCTCAAATCTATGGACCTTTC | | |
| | | | TTACCCACTGTGAAAAACCTAAAG | | |
| | | | TTACACTTAGCCCTGTTGGAC | | |
| | | | TTACCTAGTTTTCAATTGTTGATGC | | |
| | | | CACAATCATTATTTATAAGTTGAC | | |
| | | | AAAATAGTGTAGATTTCTATA | | |
| | | | CATAGTCAACAAAAAGAGTGACAT | | |
| | | | AATTATTGCCTCCAATTAAACAAG | | |
| | | | TTTGAATGAAATAAACAAACTT | | |
| | | | AGATAAACACTTCGGATGGTAGAC | | |
| | | | GTAAACAATAATATGTGGAACTCC | | |
| | | | AACATCAACACCTACCAATACC | | |
| | | | AGTAACTACTGATATTTATCATGTA | | |
| | | | CTTACCATGTACCATGTATTGTGCT | | |
| | | | ACATTACTCATGTTATCTCC | | |
| | | | CTTAATTGAGTGGCTACATACTGCT | | |
| | | | TTAGCAAATCTTCCTACTGTAACTA | | |
| | | | ATCCTCATAGATGGAAGAGT | | |
| | | | TCTCAAAACCTTAAAACTCATGCA | | |
| | | | TAAGTGGATTCATATACATATATA | | |
| | | | AAAATATATATAAATATATATA | | |
| | | | CTTTATATATATTTATATTTATATA | | |
| | | | TTTATATATTTATATTTTAATATAT | | |
| | | | TTATATAAATATATATAAAG | | |
| | | | TATAATATATATAAAGTATAAATA | | |
| | | | TATATATATTTATACTTTAAGTTCT | | |
| | | | TGGATACACGTGCAGAACATG | | |
| | | | CAGGTTTGTTACATAGGTATACAT | | |
| | | | GTGCCGTGGTGGATTGCTGCACCC | | |
| | | | ATCAACCCGTCATCTACATCAG | | |
| | | | GTATTTCTCCTAATGCTCACCCTCC | | |
| | | | TCTTATCCCCAACTACCCAAAAGG | | |
| | | | ACCTGGTGTGTGATGTTCCCC | | |
| | | | TCCCTGTGTTCATATGTTCTCATTG | | |
| | | | TTCAACTCTCACTTATGGGTAAGA | | |
| | | | ACATGCAGTGTTTGATTTTCT | | |
| | | | GTTCCTCTGTTAGTTTGCTGAGAAT | | |
| | | | GATGGTTTCCAGCTTCATCCATGTC | | |
| | | | CCTGCAAAGGACATGAACTC | | |
| | | | ATTCTTTTTTATGGCTGCATAGTAT | | |
| | | | TCCATGGTATATATGTGCCACATTT | | |
| | | | TCTTTATCCAGTCTATCATT | | |
| | | | GATGGCCATTTGAGTTGGTTCCAA | | |
| | | | GTCTTCGCTATTGTGAATAGTGCTG | | |
| | | | CAATGAACATATGTGTGCATG | | |
| | | | TGTCTTTATAGTAGAATGATTTATA | | |
| | | | ATCCTTAGGGTATACCCAGTAATG | | |
| | | | GGATTGCTGGGTTAAATGGTA | | |
| | | | TTTCTGGTTCTAGATCCTCGAGGAA | | |
| | | | TTGCCACACTGTCTTCCACAATGGT | | |
| | | | TGAACTAATTTATACTCCCA | | |
| | | | CCAACAGTGTAAAAGCATTCCTAT | | |
| | | | TTCTCCACATCCTCTCAGCATCTGT | | |
| | | | TGTTTCTTGACTTTTTAATGA | | |
| | | | TTAGCATTCTAACTGGCGTGAGAT | | |
| | | | GGTATTTCATTGTGGTTTTGATTTG | | |
| | | | CATTTCTCTAATGACCAGTGA | | |
| | | | TGATGAGTTTTTTTTCATATATTTG | | |
| | | | TTGGCCGCATAAATGTCTTCTTTTG | | |
| | | | AGAAGTGTCTGTTCGTATCC | | |
| | | | TTCACCCACTTTTTGATGGGGTTGT | | |
| | | | GTTTTTCTTGTAAATTTATTTAAGT | | |
| | | | CCCTTGTAGATTCTGGATAT | | |
| | | | TTTCCCTTTGTCAGATGGATAGATT | | |
| | | | GCAAAAATTTTCTCCCGTTCTGTAG | | |
| | | | GTTGCCCGATCACTCTGATG | | |
| | | | ATAGTTTCTTTTGCTGTGTAGAAGC | | |
| | | | TCTTTAGTTTAATCAGGTTCCATTT | | |
| | | | GTCAGTTTTGCCTTTTGTTG | | |
| | | | CAATTGCTTTTGGTGTTTTAGTCTT | | |
| | | | AAATTCTTTGCCCATGCCTATGTCC | | |
| | | | TGAATGGTATTGCCTAGATA | | |
| | | | TTCTTCTAGGGTTTTTTTTTTGGCTT | | |
| | | | TAGGTCTTGCAGTTAAGTCTTTAAT | | |
| | | | CTATCTTGAGTTAATTTTT | | |
| | | | GTATAAGATATAAGAAAGGGGTCC | | |
| | | | AGTTTCAGTTTTCTGCATATGGCTA | | |
| | | | GCCAGTTTTCCCAACACTATT | | |
| | | | TATTAAATAGGGAATCTTTTCCCCA | | |
| | | | TTGCTTGTTTTTGTCAGGTTTATCA | | |
| | | | AAGATCAGATGGTTGTAAAT | | |
| | | | GTGTGGTGTTATTTCTGAGGCCTCT | | |
| | | | GTTTTGTTCCATTGGTCTATATGTC | | |
| | | | TGTTTTTGTTCAGTACCATG | | |
| | | | CTGTTTTGTTTACTATAGCCTTGTA | | |
| | | | GTATAGTTTGAAGTCAGGTAGTGT | | |
| | | | GATGCCTCCAGCTTTGTTCTT | | |
| | | | TTTGCTTAGGATTGTCTTGGCAATA | | |
| | | | CAGGTTCTTTTTTGGTTCCATATGA | | |
| | | | AATTTAAAGTAGTTTTTTCT | | |
| | | | AATTCTGTGAAGAAAGACAATGGT | | |
| | | | AGCTTGATGGAAATAGCATTGAAT | | |
| | | | CTATAAATTACTCTCAGCAATA | | |
| | | | TGGCCATTTTCAGGATATTGATTCT | | |
| | | | TCCTATCTATGAGCATGGAATGTTT | | |
| | | | TTCCATTTGTTTGTGTCCTC | | |
| | | | TCTGGTATCCTTGAGCAGTGGTTTG | | |
| | | | TAGTTCTCATTGAAGTAGTCCTTCA | | |
| | | | CATCCCTTGTAAGTTGTATT | | |
| | | | CCTAGGTATTTTATTCTCTTTGCAG | | |
| | | | CAATTGTGAATGGGAGTTCACTCA | | |
| | | | TGATTTGGTTCTCTGTTTGTC | | |
| | | | CTATATACATATGTTGGTATATAG | | |
| | | | GAATGCTTTTATTTTAAAGATGGA | | |
| | | | AGATGATGTCTCTCTATGTAAC | | |
| | | | TCAGGCAGGTCTCAAACTCCTGGG | | |
| | | | CTCAAATGATCCTCCTACCTTAACC | | |
| | | | TCCTGAGTAGCTGAGACTTTA | | |
| | | | GTCACACACCACCATGCCTGACCA | | |
| | | | GGAATTGTTTTTCAACTTCATACTG | | |
| | | | GTAAACAAAACATATGTGTTT | | |
| | | | TCAGTTCTCATGGAACAAGCAGCT | | |
| | | | TAGTAGGAGAAACATATGTTGAAC | | |
| | | | TTGTAAGCAGAGAAGTAAATCT | | |
| | | | ATAATGACAAATCATAATTTCTGA | | |
| | | | AGGGTATTAATTAGATGTTTGAGT | | |
| | | | GAGGGGAAATATTGGAAGGTGC | | |
| | | | TCATAAGTTTATAAATGTTCTAAA | | |
| | | | ATATTTCATGCTAATCACATTAAA | | |
| | | | ATTATATCAAAGTATATAAACA | | |
| | | | TATCATGGAAAACATAATCAGCAC | | |
| | | | CATGTACTCAACACCTAGGTTAAA | | |
| | | | AAATAGCATTAAAAATTCTCTT | | |
| | | | TCCAGCTCACATTCTGCTCCCTCCC | | |
| | | | CAAATCCACAGATAACCATCGAAT | | |
| | | | TATATTTTGTTTTCTTCATTC | | |
| | | | CCTTACTTTCTTTAAGTTTTACACC | | |
| | | | CATGTATGTACCCATAAAAATCTA | | |
| | | | TTAGCTAATTTTGGTTGTGCA | | |
| | | | TGAATATTGTATCAATGCAATTAT | | |
| | | | ACTGTATATATTCTGCTTTTGCACA | | |
| | | | TATTTTTAGATTCATCCATTT | | |
| | | | GTGGCATGTAGCTTTCCATTCATTT | | |
| | | | TCACTGCTGCTCAGTATTGTATTAC | | |
| | | | AAATTTTACATTTGTTTTAG | | |
| | | | GGAAGAGTCATAAACCATCTTTAA | | |
| | | | GTTCTCCTATGTTACAAGTAATTTT | | |
| | | | GTAAATGATGTGAGGTGGTGA | | |
| | | | TTCTATTTCATTTTTTCCCATATAG | | |
| | | | ATAATTTATATTATTAATAATTCCT | | |
| | | | TCTATTTCATAAGCCAGGTT | | |
| | | | TCTATATATCTATATAAATATAGAT | | |
| | | | ATGTAGATATATGAAAGCAATATA | | |
| | | | TATATGGATGTCTTTCTGGGC | | |
| | | | TATCTGTACTTTCACACTGGCTAAT | | |
| | | | TTGCTTGTTTTTTCATCAATACTTC | | |
| | | | ACTTCCTTAATTACTACAAC | | |
| | | | ATAGCAGGGCCTGGCATCTGCTAG | | |
| | | | ATTAAATCTCTCAGCTTCTTTTTAT | | |
| | | | TAAGATTGCCCTGAATTGTCC | | |
| | | | TGGTTATCCTGGGCCCCCTACTTTT | | |
| | | | TTTATATTTTTGAATACATCTAAAT | | |
| | | | AAATTTAGAATAAATCTATT | | |
| | | | GTGTTCCATAAAACCCCTGTTGGG | | |
| | | | ATTTCAATTGAACTGCAATTAAATT | | |
| | | | TTAGATCAGTTTTGGAAGAAT | | |
| | | | TGACTCAATAGTGAGCCTTCCTAC | | |
| | | | CCAAGACCATGGCATTTATTTTCAT | | |
| | | | TTATTTATGATTTCTTTAATG | | |
| | | | CTTCTCAAAATTTTTTATTTTCTCT | | |
| | | | ATTATGGAAACGCACATTTATACT | | |
| | | | TTGACAAATTCCTAAGTACTT | | |
| | | | CTAATTTTATTGTCATTCCACATTA | | |
| | | | TCTTTTTTGTTGTTGTTTTAAAAGA | | |
| | | | CAGGGTCTCCCTCTGTCACC | | |
| | | | CAGGCTGGAGTGTACTGATGTGAT | | |
| | | | TATAGCTCACTGCAGTCTCAACCT | | |
| | | | CCTGGGCTCAAGTGATCCTCCC | | |
| | | | ACGTCAGCCTGTGGAGTAGCTAGG | | |
| | | | ACTACAGGCATGTGCCACAATGCC | | |
| | | | TGGCTCATTTTTAAGTGTTAAG | | |
| | | | TTAAAAAAAGTTGTAGAAACAGTG | | |
| | | | TTTTGCTACATTTCCCAGGCTGGTC | | |
| | | | TCAAACTCCTGGCCCCAAGCA | | |
| | | | ATCTTCCTGCCTCAGCTTCCCATAT | | |
| | | | TCGGATTATACGCATGAGGCATTG | | |
| | | | CACCAGCCCCATGTGTTATCT | | |
| | | | TTTATAAAATTTAACATTTAACTGA | | |
| | | | TAATTGATACTGTATATACATGAA | | |
| | | | TTCAATTGGTATCTATTTTTA | | |
| | | | ATATGGGAAATTTTATGCAAATCA | | |
| | | | GCACATTTTTCTCCCTTCCTTCCTT | | |
| | | | CCTTCTTTCCTTGTTCTCTTT | | |
| | | | CTTTCTCTCTCTTTCTCTTTCTCTCT | | |
| | | | TTCTTTCTTTCTTTCTTTCTCACAGG | | |
| | | | GTGTCACTCTGTTGCCCA | | |
| | | | GGCGGAGTGCAGTGGCACATGATC | | |
| | | | ATAGCTCACTCCAACCTCCAACTC | | |
| | | | AAACACTTGAGTGATCCTCTGT | | |
| | | | CCCCCGTTTCCCAAGCAGCTGGGA | | |
| | | | CTACAGGCACATGCCACGATGCCA | | |
| | | | AGCTAATTTTTAAAAATAATTT | | |
| | | | TTTTTGTAGATTCAGAGTCTTGCTA | | |
| | | | TGTTGCCCAGGCTAATCTCAAACT | | |
| | | | CCTGGCCTCAAGCAGTCCTCC | | |
| | | | CTCCTCAGCCTCCCATTACAGGCA | | |
| | | | TAAGCTGCCACTCCTGGACCTCTTT | | |
| | | | TTTTTTTTTTTTTTTTTTTTT | | |
| | | | TTGAGGCAGTCTCTCTCTGTCACCC | | |
| | | | AGGCTGGAGTATAGTGGCACGATC | | |
| | | | TCAGCTCACTGCGGGTTCAAG | | |
| | | | CAATTTTCATGCCTCAGCCTCCCAA | | |
| | | | GTAGCTGGGATTACAGGCATGGGC | | |
| | | | CACTATGCCCAGCTAATTTTT | | |
| | | | GTATTTTTCATAGAGACAGGATTTC | | |
| | | | ACCATGTTGGCTAGGCTGGTATCA | | |
| | | | AACTCCTGACTTCAGGTGATC | | |
| | | | CGCCCACTTTCACCTTCCAAAATG | | |
| | | | CTGGGATTACGTGTGAGCCACCAA | | |
| | | | ACCCAGCCCCTCATTTTCTTTT | | |
| | | | TGATTTTTATTTATTTTCCTCTGTTT | | |
| | | | TTCTTCTTTTGGATTTAGGGATGTG | | |
| | | | TGTGTGGAGGTGTATTGAG | | |
| | | | TCCGTTTTTTCTTTCTATTTGTGTGG | | |
| | | | AAATTATACACTTATTCTTTGTTAT | | |
| | | | TTTAGCAATTACTCTGGCT | | |
| | | | ATTTTAACATGCAAATATAATCAA | | |
| | | | GTTTAGAATTAGCCATTTTTTATAA | | |
| | | | CTCTCCTTCTGACTAGTTGAA | | |
| | | | GAAATGAGAATGCTTTAACATCAA | | |
| | | | ACAGCCAACTCTTTACTTATACACT | | |
| | | | ATTGCTATTCATTATAGCATT | | |
| | | | TTTAGTCTAGCTTCCTCCCTCCTCT | | |
| | | | TTCTCTCTCTCTCTCTCTGTCTCTCT | | |
| | | | CTCTCTCACTAATGTTTGC | | |
| | | | TATTTCTCCCTACAATTCAGAATTT | | |
| | | | TATTTATGGATGAAGTACATATAT | | |
| | | | AATTTATTACAATTCATTTTA | | |
| | | | ATGAAAAACTTTTAGTGGTAAATT | | |
| | | | GTATTAGTCTTTGGGAAAAAACAT | | |
| | | | TTATTGATACCATTTTCTCATT | | |
| | | | ACTTAAAAATAGTTTCACTTCATAT | | |
| | | | AGAATTCTATGTCGACAGTAATTTT | | |
| | | | CTTTCAGGAAGTAGAAAATA | | |
| | | | TTAACTTACACTATTTTGGCTTCCA | | |
| | | | TTACTGCTGTTAAGCATTCAGATCA | | |
| | | | TCAGAGAAATGCAAATCAGA | | |
| | | | ACCACAATGAGATGCCATCTCATG | | |
| | | | CCAGTCAGAATGGCAATCATTAAA | | |
| | | | AAGTCAGGAAACAATAGATGCT | | |
| | | | GGTGAGGCTGTGGAGAAATAAGA | | |
| | | | ATGCTTTTACACTGTTAGTGGGAAT | | |
| | | | GTAAATTACTTCAACCATTGCT | | |
| | | | CTTAAGGGCTCTTTGTCTTTAATGA | | |
| | | | TCCGCATTTTTATTATGATGTGTCT | | |
| | | | AGGCAGTTATTATTGTGTTT | | |
| | | | ATTCTATTTCTTTATTTGCTGTCTAT | | |
| | | | CCAAGATTTGAGGATTAATTTTTTA | | |
| | | | ATTTCTAGAAAATTCAGAA | | |
| | | | GTATTATTTATTTATTCAATTATTA | | |
| | | | CCTCTTTCTATTATTTCCTTTTTAAA | | |
| | | | ATAAAAGGGTATATGTTAG | | |
| | | | AATTTTTCACTCTCTCCTTTATGCC | | |
| | | | TTTAACTTCATATTTTCTATTTCTTT | | |
| | | | GAATTTCTGGGCTGCATTC | | |
| | | | TTAAGAATTCTAAAACATATATTTT | | |
| | | | AGTTTCTAAAAGTTTCATTAGATTT | | |
| | | | CTGTTCAAAATTCCTTCCAT | | |
| | | | TTGTGATCTTTCGAATGTGCTTCTG | | |
| | | | CTTTAGGCATTAGTAGTGGACATT | | |
| | | | CTGGTTCCCCATTGAGCTTCC | | |
| | | | CTGCATCAGCTGTTTTGCCTGGTGG | | |
| | | | CTGCCACCAACGCTTTTAGCTACCT | | |
| | | | CCCTCCTCAAACTTTGGGGT | | |
| | | | CAGGCCACACACTATAAAGGATTG | | |
| | | | GAAAAAAAAATGAAAATATGAAA | | |
| | | | AACTTACACTTTGTATCAGTCAG | | |
| | | | CAGAAGGATAATCTTCACACTACA | | |
| | | | GTTTATGCTTCAGAAGCCACCCCTT | | |
| | | | CTCTGTGGATTAGACCATGAC | | |
| | | | TAGAAGTTTCCTGAGACCATCCCTT | | |
| | | | GCCCAGCTCTTTTGCTGATCCCCTT | | |
| | | | CACTTCCTCTGTTACAGGTT | | |
| | | | TCCCTGATGAGCACTCCTTCAATA | | |
| | | | AAACATAGTCATCCAAATCCCAAT | | |
| | | | CTCAAGCACGGTGTCACGGGAA | | |
| | | | CCTGATCTAAGTCAGCATTTTCTTT | | |
| | | | ATTCTTAATCACAACTAGTTGATA | | |
| | | | GTCCATATCTAATATATAATG | | |
| | | | AATGTACAGTTGTTTCTGTTGGAGT | | |
| | | | TCACATATGATGCCTTGTTTCCTTT | | |
| | | | GTAGTTTTGTGATTGATAGC | | |
| | | | TTCGAACTGCTCATTTACCTTGACC | | |
| | | | TTTTGAATTCTTTGAAAACTGAGTT | | |
| | | | AAGTCTGATTTTCCAGAGTT | | |
| | | | TTTATGTTTGCTTCTGTCAGTTGCA | | |
| | | | GAGAATCAATGAGAAGAACACTTT | | |
| | | | AAATTCTTGTTTTCGGTTTTT | | |
| | | | TTCCAATCACATAAGTAGGATTTA | | |
| | | | CCTGAATATATATATAATATATAA | | |
| | | | ACATATATTTATATAAAATAAA | | |
| | | | AACATATAAAATATGAAATATATA | | |
| | | | ATACAGTATAAAATCTATTTTATGT | | |
| | | | AAAATCTATTTTATGTAAACA | | |
| | | | TGATAATTAAATATATATTTAAAT | | |
| | | | AATATAAATATAATAAATATTTGA | | |
| | | | AGCAATTGTATTTTTTAAAAAT | | |
| | | | TTCTTCTAAAGAAAACCAGGATAC | | |
| | | | ATGTGCAGAACCTGCAGGTTTGTT | | |
| | | | ACATAGGTATACGTGTGCCATG | | |
| | | | GTGGTTTGCTGCACCTATTGACCCG | | |
| | | | TCCTCTAAGTTCCCTCCCCTCACCT | | |
| | | | CCCACCCCCCAGCAGGCCCT | | |
| | | | GGTGTGTGTTGTTCCCCTCTCTGTG | | |
| | | | TCCATGTATTCTCGCCTCCCACTTA | | |
| | | | TGAGTGAGAACACGCGATGT | | |
| | | | TTGGTTTTCTGTTCCTGTGTTAATT | | |
| | | | TGCTGAGGATGATAGCTTCCAGCT | | |
| | | | TCATCCACGTCCCTGCAAAGG | | |
| | | | ACATGATCTCATTCCTCAATACTAT | | |
| | | | GGCTACGTAGTATTCCATGGTGTA | | |
| | | | TATATACCACATTTTCTTCAT | | |
| | | | CCAGTCATGCAAATTTATATGAAT | | |
| | | | GTCAATTCTTTTATAGTGATCTTCT | | |
| | | | GGGGCTATTACAATATATAGG | | |
| | | | GCTGTTTTTTTAAAACTAATTATAT | | |
| | | | TTATTTCATGTTGCTTTAACTTATT | | |
| | | | AAAAAACAGACTGAAGAAAG | | |
| | | | ACTGGGTGTGAAGTCAGTAAATTA | | |
| | | | ATTTCAAATTAAATAAACTTTTCTA | | |
| | | | CAGCTATTTTATGCTCAATAA | | |
| | | | CTTTCTACTTATTCTTGAGTTCAAA | | |
| | | | ACTATATGGGTTCACATTTAAATTA | | |
| | | | TATAGTGTATTTTCTCCATA | | |
| | | | AACTGAAGTTGTTAGAACATTGAT | | |
| | | | TTTTTTAAGTAAATGGATTTTTGCA | | |
| | | | CCACTTCAAGAAAGAAACCTT | | |
| | | | CAAACAGCCTGGAAATATCACATC | | |
| | | | AATAAAGCACAACCTGGGAATCAA | | |
| | | | AGTATTAGGGTACCTTGTTACT | | |
| | | | GAGATTATGGATGTGATGCTTCTG | | |
| | | | TGGGCCATTAGCATGTGCACTGTG | | |
| | | | TGTATGATATGCTCTATGTTCT | | |
| | | | CTTCCCACTAATAATTTTATTTTTA | | |
| | | | ATTTCAGCAAGATTTAGTCTCAAA | | |
| | | | TAACACAATAATAATGGAGGT | | |
| | | | CATTGTGAAGTAGTGGATGTAAAT | | |
| | | | AGATCTGATGTGGTTTTGGTTTATT | | |
| | | | GCAGTAATTGTTTTCACTAAT | | |
| | | | TCTCTAGTTTTTCAACTTTTGATTG | | |
| | | | TTTAAGATGGTTCTTGAGTCCTTTT | | |
| | | | GACATGACCCTATCTATTTT | | |
| | | | TGATAACTTCATAGCCTTTAGTATA | | |
| | | | AAAACAGGTAGGCTTATATTACAT | | |
| | | | ATTTCCAACTTCAAACTTGTT | | |
| | | | ATTTATTTATCTAAGACTATACAGT | | |
| | | | TCTTTTCAGAGAAAAACCTTCTTTA | | |
| | | | TAAACCAGAATCTTAACAGG | | |
| | | | AAGAGTGCTCATTTTAATTGAGCT | | |
| | | | GATCATGTTTCTAGGATTTTTTAGT | | |
| | | | TAAAAGAAAATACATATTTTA | | |
| | | | AAAATATAAATTATATTTTTATTTC | | |
| | | | ATAGTGGTATTTTCAATTTTGTCTG | | |
| | | | GGATAATAAGATGTTTTATT | | |
| | | | TAACTTGTTTGATTTTGTAGTTTTA | | |
| | | | TCTTTGTGGGAAGGACCTGGTAAG | | |
| | | | AGGTAATTGAATCATGGGGGC | | |
| | | | AGGACTTTCCCATGATGTTCTCATG | | |
| | | | ATAATGAATAAGTTTCATGAGATC | | |
| | | | TGTTGGTTTCATAATGTGGAG | | |
| | | | TTTCCCTGCAAAGGCTCTTGTCCTG | | |
| | | | TCTGTGCCATTTGAGACATGCCTTT | | |
| | | | CAACTTCTGCCCTGATTGTG | | |
| | | | AGGCCTCCCCCGCCATGTGGAATT | | |
| | | | GGGTCTTACTTTTGTAAATTGCCCA | | |
| | | | GTCTCAGGTATGTCTTTATCA | | |
| | | | GCAGCCTGAAAACTGACTAATATA | | |
| | | | GTAAGTTGGCACCAGTAGAGAGGG | | |
| | | | GCACTGCTGAAAAGGTACCCGA | | |
| | | | ATATGTGGAAGCAACTTTAAACTG | | |
| | | | GGTAACAGGCAGAGGTTGGAATGG | | |
| | | | TTTGGAGGGCTCATAAGAAGAC | | |
| | | | AGGAAAGTGTGGGAAATTTGGAAC | | |
| | | | TCCCTAGAGACTTGTTGAATGGCTT | | |
| | | | TAACCAAAATGCTGATAATAA | | |
| | | | TATGAACAATGAAGTCCAGGCTGA | | |
| | | | GGTGGTCTCAGACAAAGATAAGGA | | |
| | | | ACTTCTTGGGAACTGGAGCAAA | | |
| | | | CGTGACTCTTGTTATGTTTTAGACA | | |
| | | | TAAAGCAAAGAGACTGGAGGCATT | | |
| | | | TTGCCCCTGCCCTAGAGATTT | | |
| | | | GTGGGACATTAAACTTGAGACAGA | | |
| | | | TTATTTAGGGTATCTGGAGGAAGA | | |
| | | | AATTTTTATGCAGCAAAGCATT | | |
| | | | CAAGAGGTGACTTGGTTGCTATTA | | |
| | | | AAGGCATTCAGTTTTAAAAGGGAA | | |
| | | | ATACAGCATAAAAGTTCAGAAA | | |
| | | | ATTTTCAGCCTGACAATGCAGTAG | | |
| | | | AAAAGGAAAACCAATTTTCTGAGG | | |
| | | | AGAAATTTAAGCTGGCTGCAGA | | |
| | | | CATTTACATAAGTAACAAGAAGCT | | |
| | | | GAATGTTAATCACTAAGACAATGA | | |
| | | | GGAAAATGTCTCCAGGGCATGT | | |
| | | | CAGAGACCTTTGTGGCAGCCCCTC | | |
| | | | CCATCACAGACCAGGAGCTTTAGA | | |
| | | | AGGAAAAATGGCTTCGTGGGCT | | |
| | | | GGTCACAGGGTCCCTCTGCTGTGT | | |
| | | | GCAGTCTAGGGACTTGGTGCCCTG | | |
| | | | TGTCCCAGCAGCTCCATCCATG | | |
| | | | ACTAAAAGGGGCCAAGGTACAGCT | | |
| | | | TGGGCTGTGGCTTCAGAGGGTGGA | | |
| | | | AGCCCCAAGTCTTGCCAGCTTC | | |
| | | | CATATGGTGTTGAGCCTGGGTTCA | | |
| | | | CAGAAGTCAAGAACTGAGGTTTGG | | |
| | | | GAACTTACACCAAGATTTCAGA | | |
| | | | GGATGTATGGAAATGCCTGGATGC | | |
| | | | CCAGGCAGAAGTTTGCTGCAGGGG | | |
| | | | CAAGGCCCTCATGGAGAACCTC | | |
| | | | TGCTAGGGCAGTGAAGAAGGGAA | | |
| | | | AAGTATGGTGGGAGCCCCCATACA | | |
| | | | GAGTCCCTACTGAGGCACCACCT | | |
| | | | AGTGGAGCTTTGAGAAGAGGGCCA | | |
| | | | CTGTCCTCCAGAACTCAGGATGGT | | |
| | | | AAATCCACCACGCACCTGGAAA | | |
| | | | AGCTGCAGACAATTCCAGCCTGTT | | |
| | | | AAAGCAGCCAGGAGGGGGCTATA | | |
| | | | CCCTGCAAAGCCACAGGGGCGGA | | |
| | | | CCTGCTCAAGGCTGTGGGAGACCA | | |
| | | | CCTCTTGCATCAGTGTGACCTGGAT | | |
| | | | GTGAGACATGGAGTCAAAGGA | | |
| | | | GATCATTTTGGAGCTTTAAGATTTG | | |
| | | | ACTGCCCCACTGGATTTCAGACTTT | | |
| | | | CATGGGGCCTGTAGCCCCTT | | |
| | | | CGTTTTGGCCAATGCCTCCCATTTG | | |
| | | | GAGTGGCTGTATTTACCCAATGCC | | |
| | | | TGTATCCCCATTGTATCTAGG | | |
| | | | AAGTAACTAACTTGCTTTTGATTTT | | |
| | | | ACAGGCCCATAGGTGGAAGGGCG | | |
| | | | ATGTTTCTTTCTGGACGCTCCA | | |
| | | | GGGAGAACTCTGTTTTCTTACCTTT | | |
| | | | TCTGGATTCTAGAGGCTTCCCACA | | |
| | | | ATCCTTGGCTTAAGGTCCATC | | |
| | | | TTTAAGCTTTGTCTCTGATGAGACT | | |
| | | | TTGGACTGCGGACTTTTGAGTTAAT | | |
| | | | GCTGAAATGAGTTAAGACTT | | |
| | | | TGGGTGACTGTTGCGAAGACATGA | | |
| | | | TTGGTTTTGAAATGTGAGAACATTT | | |
| | | | AAGAGGGGCCAGGGGCAGAAT | | |
| | | | GATATGGTTTGACTTTGTCCGCAGT | | |
| | | | CAAATCTCATCTTGAATTTCTATGT | | |
| | | | GTTTGGAGAGGTACCCGGTG | | |
| | | | GGAGGTAATTGAATCATGAGGGCA | | |
| | | | GGTCTTTTCTGTGCTGTTCTCATGA | | |
| | | | TGGTGAGTAAGTCTCATGAGA | | |
| | | | TCTGATGGTTTTATAAAGGGGAGT | | |
| | | | TTCCCTGCCCAAGTTCTTCTCTTGT | | |
| | | | CTGCCATCATGTGCGATGTGC | | |
| | | | CTTTCACCTCTGCCATGATTATGAG | | |
| | | | GCCTCCCTGGCCATGTGGAACTGT | | |
| | | | GAGTCCATTAAACCTCTTTCT | | |
| | | | TTTGTAAATTGCCCAATCTTGGGA | | |
| | | | ATGTCTTTATCAGCAGTGGGAAAA | | |
| | | | CGGATTAATATACTAATTTATA | | |
| | | | GCTAGTAGGTAAAAAGCCAGGGAC | | |
| | | | TTGCCATTAGCGTTGGAAGTGGGG | | |
| | | | TTGTGGGGGCAGTCTTGTGGAA | | |
| | | | CTGAGCCCTTAACCTGTGGGGTTG | | |
| | | | AATGATATCTCCAGGTATATCATG | | |
| | | | TCAGAATTGAATTCAATTAGAG | | |
| | | | GATACCTAGCTTGCGTTCAATGCA | | |
| | | | GAATTGCTTGCTGGTGAGGACAAA | | |
| | | | TCCCTATACACATTTTGGTGAC | | |
| | | | CAGAGGTAAAGCATTTTATGTTGA | | |
| | | | TTCTTGAGTGAGAGAGTAGAAATA | | |
| | | | ACACTGGTTTTTTCCCTATGTC | | |
| | | | CTTACAACCACCAATTGGATACAT | | |
| | | | TGTTTCAGTATTTTGAAATTTTTCA | | |
| | | | TTTAATTTTTATAAATTTTCT | | |
| | | | TTTTAAATTTTAGATTCTACAATAT | | |
| | | | CTCCAATTCTTCAGTTTATTCCCTC | | |
| | | | TTACTATGTATAAGTATTTC | | |
| | | | CCCAAGTTTCACTTTATCTTTCTAT | | |
| | | | TACTTTTTTTACATAATAGAGCTAT | | |
| | | | AAAGGCAATTCACAATTCTC | | |
| | | | TCTTTTCTCATATATAATATAGAGC | | |
| | | | ATATTATAAATACTCTACTTTGGAA | | |
| | | | AATTATTCTTTATAGGAAAT | | |
| | | | TACAGATAATATTTGATGAAGAAA | | |
| | | | ATCGAATATAATCATTTTTCAATAC | | |
| | | | TTAGGATAACAGATTCAGGCA | | |
| | | | AAGATAAAACATTAAAGGAAAAG | | |
| | | | TTAGTGAAAACTATTAATATATAG | | |
| | | | TGGAGGCATCACGTTGTTATGAA | | |
| | | | CTTCATTCATCAATACTGATACCAC | | |
| | | | TAAAAATGGAACAACATGTAATTA | | |
| | | | TGTGCTCAATGTGATGAATAT | | |
| | | | GAAGTAGACTGCACCACTCTGCAG | | |
| | | | TACAGTCACGAAATAAGAAACCAA | | |
| | | | CTCCAATCAAAATACCCCTAAA | | |
| | | | GCTACCTTCCAGTTTATAAAAAGT | | |
| | | | ATGAAGAATAGAGGGGCAATTAA | | |
| | | | ATGATACCATAAAGAGTCAAATA | | |
| | | | CAGGGCATGCAACATAGCTGCTGA | | |
| | | | TTGGATTTATTCAACATGTCAGTGG | | |
| | | | CATGAATACAATAGGAGGCAG | | |
| | | | GTAGGGAGAAGGCACTACCCTGAA | | |
| | | | TTATGAGACTGAAGAGATATAATA | | |
| | | | AACAAATGCAATGTGTGGACTT | | |
| | | | GGTTGGGATCTTCATTCAAAGACC | | |
| | | | AACTATAAAAAGACATTGTTGTGA | | |
| | | | GAATTGAGGAAATTTGAATGAG | | |
| | | | AAATGTATTTTTATCTAATTTGTTA | | |
| | | | GCTGTGATAATAGTATTGTGGGAG | | |
| | | | TAAGAAGCTATTCATATTTCT | | |
| | | | ATATATATATACCAAGTACATAGG | | |
| | | | AGTGAAATAATACAAAATCTGGAA | | |
| | | | TTTGCCTTAAAATTCCTCTGCA | | |
| | | | AAATTATAAAAAAGAACGATGACA | | |
| | | | AACTAAAAAGGTGTAGTATTCTTC | | |
| | | | TATGGCTGCTATAACAAATGAC | | |
| | | | CAAAAAACATAGTGACTGAAAATA | | |
| | | | ACCCACATTTATTATCTTACAGTTC | | |
| | | | CATAGGTTAGAAGTTCAACAT | | |
| | | | GGGTCTCATGAGATCAAAAGCAAG | | |
| | | | GCCTTGGCAGGGTGACGTTTCTTTC | | |
| | | | TGGAGGTTCCAGGGGGAACTC | | |
| | | | TGTTTTCTTACTTTTTTTAGATTCTA | | |
| | | | GAGGCTTCCCACAATCCTTGGCTT | | |
| | | | AAGGTCCATCTTTAAAGACA | | |
| | | | GCAACGTTTCATCTCTCTACCTATT | | |
| | | | CTTTCATCCTTACATCTTTCTCTAA | | |
| | | | CTATTCCTTTTCTTCTGTCT | | |
| | | | TCCACTTTTAAGAGCCTTTTTGAGT | | |
| | | | CTATTGAGGCCAACTGGACAATCA | | |
| | | | AGGATTATCTCCCTATGTTAA | | |
| | | | GGTCAATTGATTAGTGACCTAATT | | |
| | | | CCATCTACAATCACAATTCCTCTTT | | |
| | | | GCCATATAATGTAAAATATTC | | |
| | | | ATACCTCTAAGGATTAGGACATGG | | |
| | | | ACATCTTTGAGGGTCATTAGTCATC | | |
| | | | TTACCACAGGAAGGAAGGAAG | | |
| | | | GAAGGAAGGAAGGAAGGAAGGAA | | |
| | | | GGAAGGAAGGAAGGAAAGGGAGG | | |
| | | | AGAGGAGAGGAGAGGTAGGAGGG | | |
| | | | A | | |
| | | | AGAAGAAAAAAATAGTATGAAAA | | |
| | | | AATCTTGATAAATTTGAAAACTGG | | |
| | | | GTGAATAATATGTGGAATTCTCT | | |
| | | | CTATTTTTGTTAATGTTGGAAAATT | | |
| | | | TAATAAAAACAATGAACAGTGA | | |
| | | | (SEQ ID NO: 31) | | |
| ENTPD1 | Ecto- | NM_001776.6 | ACCGAGACGGACCACAGCAAGCA | NP_001767.3 | MEDTKESNVKTFCSKNI |
| | nucleoside | | CAGGCTGGGGGGGGGAAAGACCA | | LAILGFSSHAVIALLAVG |
| | Tri- | | GGAAAGAGGAGGAAAACAAAAGC | | LTQNKALP |
| | phosphate | | T | | ENVKYGIVLDAGSSHTS |
| | Diphospho- | | GCTACTTATGGAAGATACAAAGGA | | LYTYKWPAEKENDTGV |
| | hydrolase | | GTCTAACGTGAAGACATTTTGCTC | | VHQVEECRVKGPGISKF |
| | 1 | | CAAGAATATCCTAGCCATCCTT | | VQKVNEIG |
| | | | GGCTTCTCCTCTATCATAGCTGTGA | | IYLTDCMERAREVIPRS |
| | | | TAGCTTTGCTTGCTGTGGGGTTGAC | | QHQETPVYLGATAGMR |
| | | | CCAGAACAAAGCATTGCCAG | | LLRMESEELADRVLDV |
| | | | AAAACGTTAAGTATGGGATTGTGC | | VERSLSNYP |
| | | | TGGATGCGGGTTCTTCTCACACAA | | FDFQGARIITGQEEGAY |
| | | | GTTTATACATCTATAAGTGGCC | | GWITINYLLGKFSQKTR |
| | | | AGCAGAAAAGGAGAATGACACAG | | WFSIVPYETNNQETFGA |
| | | | GCGTGGTGCATCAAGTAGAAGAAT | | LDLGGAS |
| | | | GCAGGGTTAAAGGTCCTGGAATC | | TQVIFVPQNQTIESPDN |
| | | | TCAAAATTTGTTCAGAAAGTAAAT | | ALQFRLYGKDYNVYTH |
| | | | GAAATAGGCATTTACCTGACTGAT | | SFLCYGKDQALWQKLA |
| | | | TGCATGGAAAGAGCTAGGGAAG | | KDIQVASNE |
| | | | TGATTCCAAGGTCCCAGCACCAAG | | ILRDPCFHPGYEKVVNV |
| | | | AGACACCCGTTTACCTGGGACCCA | | SDLYKTPCTKRFEMTLP |
| | | | CGGCAGGCATGCGGTTGCTCAG | | FQQFEIQGIGNYQQCHQ |
| | | | GATGGAAAGTGAAGAGTTGGCAG | | SILELFN |
| | | | ACAGGGTTCTGGATGTGGTGGAGA | | TSYCPYSQCAFNGIFLPP |
| | | | GGAGCCTCAGCAACTACCCCTTT | | LQGDFGAFSAFYFVMK |
| | | | GACTTCCAGGGTGCCAGGATCATT | | FLNLTSEKVSQEKVTEM |
| | | | ACTGGCCAAGAGGAAGGTGCCTAT | | MKKFCAQ |
| | | | GGCTGGATTACTATCAACTATC | | PWEEIKTSYAGVKEKYL |
| | | | TGCTGGGCAAATTCAGTCAGAAAA | | SEYCFSGTYILSLLLQGY |
| | | | CAAGGTGGTTCAGCATAGTCCCAT | | HFTADSWEHIHFIGKIQ |
| | | | ATGAAACCAATAATCAGGAAAC | | GSDAGW |
| | | | CTTTGGAGCTTTGGACCTTGGGGG | | TLGYMLNLINMIPAEQP |
| | | | AGCCTCTACACAAGTCACTTTTGTA | | LSTPLSHSTYVFLMVLF |
| | | | CCCCAAAACCAGACTATCGAG | | SLVLFTVANGLLIFHKPS |
| | | | TCCCCAGATAATGCTCTGCAATTTC | | YFWKD |
| | | | GCCTCTATGGCAAGGACTACAATG | | MV (SEQ ID NO: 34) |
| | | | TCTACACACATAGCTTCTTGT | | |
| | | | GCTATGGGAAGGATCAGGCACTCT | | |
| | | | GGCAGAAACTGGCCAAGGACATTC | | |
| | | | AGGTTGCAAGTAATGAAATTCT | | |
| | | | CAGGGACCCATGCTTTCATCCTGG | | |
| | | | ATATAAGAAGGTAGTGAACGTAAG | | |
| | | | TGACCTTTACAAGACCCCCTGC | | |
| | | | ACCAAGAGATTTGAGATGACTCTT | | |
| | | | CCATTCCAGCAGTTTGAAATCCAG | | |
| | | | GGTATTGGAAACTATCAACAAT | | |
| | | | GCCATCAAAGCATCCTGGAGCTCT | | |
| | | | TCAACACCAGTTACTGCCCTTACTC | | |
| | | | CCAGTGTGCCTTCAATGGCAT | | |
| | | | TTTCTTGCCACCACTCCAGGGGGA | | |
| | | | TTTTGGGGCATTTTCAGCTTTTTAC | | |
| | | | TTTGTGATGAAGTTTTTAAAC | | |
| | | | TTGACATCAGAGAAAGTCTCTCAG | | |
| | | | GAAAAGGTGACTGAGATGATGAA | | |
| | | | AAAGTTCTGTGCTCAGCCTTGGG | | |
| | | | AGGAGATAAAAACATCTTACGCTG | | |
| | | | GAGTAAAGGAGAAGTACCTGACTG | | |
| | | | AATACTGCTTTTCTGGTACCTA | | |
| | | | CATTCTCTCCCTCCTTCTGCAAGGC | | |
| | | | TATCATTTCACAGCTGATTCCTGGG | | |
| | | | AGCACATCCATTTCATTGGC | | |
| | | | AAGATCCAGGGCAGCGACGCCGG | | |
| | | | CTGGACTTTGGGCTACATGCTGAA | | |
| | | | CCTGACCAACATGATCCCAGCTG | | |
| | | | AGCAACCATTGTCCACACCTCTCT | | |
| | | | CCCACTCCACCTATGTCTTCCTCAT | | |
| | | | GGTTCTATTCTCCCTGGTCCT | | |
| | | | TTTCACAGTGGCCATCATAGGCTT | | |
| | | | GCTTATCTTTCACAAGCCTTCATAT | | |
| | | | TTCTGGAAAGATATGGTATAG | | |
| | | | CAAAAGCAGCTGAAATATGCTGGC | | |
| | | | TGGAGTGAGGAAAAAAATCGTCCA | | |
| | | | GGGACCATTTTCCTCCATCGCA | | |
| | | | GTGTTCAAGGCCATCCTTCCCTGTC | | |
| | | | TGCCAGGGCCAGTCTTGACGAGTG | | |
| | | | TGAAGCTTCCTTGGCTTTTAC | | |
| | | | TGAAGCCTTTCTTTTGGAGGTATTC | | |
| | | | AATATCCTTTGCCTCAAGGACTTCG | | |
| | | | GCAGATACTGTCTCTTTCAT | | |
| | | | GAGTTTTTCCCAGCTACACCTTTCT | | |
| | | | CCTTTGTACTTTGTGCTTGTATAGG | | |
| | | | TTTTAAAGACCTGACACCTT | | |
| | | | TCATAATCTTTGCTTTATAAAAGAA | | |
| | | | CAATATTGACTTTGTCTAGAAGAA | | |
| | | | CTGAGAGTCTTGAGTCCTGTG | | |
| | | | ATAGGAGGCTGAGCTGGCTGAAAG | | |
| | | | AAGAATCTCAGGAACTGGTTCAGT | | |
| | | | TGTACTCTTTAAGAACCCCTTT | | |
| | | | CTCTCTCCTGTTTGCCATCCATTAA | | |
| | | | GAAAGCCATATGATGCCTTTGGAG | | |
| | | | AAGGCAGACACACATTCCATT | | |
| | | | CCCAGCCTGCTCTGTGGGTAGGAG | | |
| | | | AATTTTCTACAGTAGGCAAATATG | | |
| | | | TGCTAAAGCCAAAGAGTTTTAT | | |
| | | | AAGGAAATATATGTGCTCATGCAG | | |
| | | | TCAATACAGTTCTCAATCCCACCC | | |
| | | | AAAGCAGGTATGTCAATAAATC | | |
| | | | ACATATTCCTAGGTGATACCCAAA | | |
| | | | TGCTACAGAGTGGAACACTCAGAC | | |
| | | | CTGAGATTTGCAAAAACCAGAT | | |
| | | | GTAAATATATGCATTCAAACATCA | | |
| | | | GGGCTTACTATGAGGTAGGTGGTA | | |
| | | | TATACATGTCACAAATAAAAAT | | |
| | | | ACAGTTACAACTCAGGGTCACAAA | | |
| | | | AAATGCATCTTCCAATCCATATTTT | | |
| | | | TATTATGGTAAAATATACATA | | |
| | | | AATATAATTCACCATTTTAACATTT | | |
| | | | AATTCATATTAAATACGTACAAAT | | |
| | | | CAGTGACATTTACTACATTCA | | |
| | | | CAGTGTTGTGCCACCATCACCACT | | |
| | | | ATTTAGTTCCAGAACATTTGCATCA | | |
| | | | TCAATACATTGTCTAGAGACA | | |
| | | | AGACTATCCTGGGTAGGCAGAAAC | | |
| | | | CATAGATCTTTTGTGTTTACACCTA | | |
| | | | TGGAAACCAACTGTACCATAA | | |
| | | | AGATAGTTCACTGAGTTTTAAAGC | | |
| | | | CAAGCCACATCTTATTTTTCCAAGG | | |
| | | | TTTAATTTAGTGAGAGGGCAG | | |
| | | | CATTAGTGTGGAGTGGCATGCTTTT | | |
| | | | GCCCTATCGTGGAATTTACACATC | | |
| | | | AGAATGTGCAGGATCCAAGTC | | |
| | | | TGAAAGTGTTGCCACCCGTCACAC | | |
| | | | AACATGGGCTTTGTTTGCTTATTCC | | |
| | | | ATGAAGCAGCAGCTATAGACC | | |
| | | | TTACCATGGAAACATGAAGAGACC | | |
| | | | CTGCACCCCTTTCCTTAAGGATTGC | | |
| | | | TGCAAGAGTTACCTGTTGAGC | | |
| | | | AGGATTGACTGGTGATGTTTCATTC | | |
| | | | TGACCTTGTCCCAAGCTCTCCATCT | | |
| | | | CTAGATCTGGGGACTGACTG | | |
| | | | TTGAGCTGATGGGGAAAGAAAAGC | | |
| | | | TCTCACACAAACCGGAAGCCAAAT | | |
| | | | GTCCCCTATCTCTTGAATGATC | | |
| | | | AAGTCACTTTTGACAACATCCAGG | | |
| | | | TGAATATAAAAACTTAATAAAGCT | | |
| | | | GTGGAAAGGAACTCTTAATCTT | | |
| | | | CTTTTCTGCTACTTAGGTTAAATTC | | |
| | | | ACTAGATCTTGATTAGGAATCAAA | | |
| | | | ATTCGAATTGGGACATGTTCA | | |
| | | | AATTCTTTCTTGTGGTAGTTGCCTA | | |
| | | | TACTGTCATCGCTGCTGTTGGTTGA | | |
| | | | GCATTTGTGGTGTACCACGC | | |
| | | | TGTGTGCTCAAGGGTATTACATTC | | |
| | | | ATCTTCTCATTTAATCCTCACAACA | | |
| | | | ATCTGAAGAAGGTAGGTATTA | | |
| | | | CAATTCCCACTTCATACAAACACA | | |
| | | | AACTGAGGTTCAGAGAGGTTAAGT | | |
| | | | CATTTGCCCAAATGGCTGAGCC | | |
| | | | AAAGCCTACCATGTACCTAACCTT | | |
| | | | TATTTTCTTTCCCGAACATACCAGG | | |
| | | | CTGTCTCCTCATAACTTCCAA | | |
| | | | GCATGCACTTAAAACTCCACATGA | | |
| | | | ATACAAGGTTCATGGGACTTGGTA | | |
| | | | TTCATAGAAAGGGAGGCAGAAA | | |
| | | | GCTGGTCTGTTCCTGATAGGCTTGT | | |
| | | | AATTTAATATCATTCTGTTCATGTG | | |
| | | | CTTTGGATGGAAGCACATCT | | |
| | | | GGCATATGATGCTAATCAGTGGTT | | |
| | | | CCCATACCCCTGGCTTCCTAATTTT | | |
| | | | AATGTTTGCTTCACAGCATAGT | | |
| | | | AGATTGACATCAAATAGTGGCCGA | | |
| | | | TGATGATGAAAATAAAGGTCAAAT | | |
| | | | AAGTTGAGCCAATAACAGCCGC | | |
| | | | TTTTTTCCTTCTGTCTGCGTATACA | | |
| | | | AAGCACTGTCATGCACACAATCTA | | |
| | | | TTCTGACCCTCACAACAACCC | | |
| | | | ATAAGGGTGTAAATAGTATTTCCA | | |
| | | | TTTTACAAATGAGGATCACACAAA | | |
| | | | CTACTACATGGCAGAGCAGATA | | |
| | | | CTCCAACTCATGTCTTCTGGTTGAA | | |
| | | | GCCTATTGCTTTTTCTTTTCTAAAC | | |
| | | | ACTTTCCCTCAGCAAGTTGG | | |
| | | | AATTAGACTTCACAAGTCTCCTTCA | | |
| | | | GAGAACACAAATCTTTTCTTATTCC | | |
| | | | ATTCCTGTTTGGTTGCCTAC | | |
| | | | GTCCAATCTCCCCCTCCCCAGAGA | | |
| | | | TGCCAAAAAAAAAATCCTTTAAGG | | |
| | | | TATTTGGGAGCCAAACTCAACT | | |
| | | | TGTTAAAATCTCAAATTATGGAGA | | |
| | | | CAATCAGCAGACACAACCTAACCC | | |
| | | | CAATTATTTTGGCAGGAAGGTT | | |
| | | | GGTTTAGAGGCAGATCCAGCAATC | | |
| | | | TGCTTTGGGCCACTCTGGGTGGGG | | |
| | | | TAGGTGAAATAACATTGGTCAC | | |
| | | | TGTTAACTAATTTTAATATTGGATT | | |
| | | | GGCCATTGGTTATCACTGATTACC | | |
| | | | ATTCTCCCCTGGATTTTCACC | | |
| | | | CAGGACTCAAAACTTGGTTCTGCT | | |
| | | | AACCCTGTTCCTTTATGAGGAACCT | | |
| | | | TTTAAAGATTCCTTTATAAGG | | |
| | | | TGGGAGTTTTTTTTCTATGAACCTA | | |
| | | | TAGGGGAGAAAAAAGATCAGCAG | | |
| | | | AAGTCATTACTTTTTTTTTTTT | | |
| | | | TTTTTTTTTTTTTTGAGAGAGAGTC | | |
| | | | TCACTCCATTGCCCAGGCTGGAGT | | |
| | | | GCAGTGGTGCTATCTCGGCTC | | |
| | | | ACTGCAACCTCCGCCTCCTGGGTT | | |
| | | | CAAGCAATTCTCCTGCCTCAGCCT | | |
| | | | CCCGAGTAGCTGGGATTGCAGG | | |
| | | | TGCCCACCACCACACCCGGCTAAT | | |
| | | | TTTTGTATTTTTAGTAAAGACAGGG | | |
| | | | TTTCACCATGTTGGCCAGGCT | | |
| | | | CGTCTCCAACTCCCAATCTCAGGT | | |
| | | | GATCCTATTGCCTCGGGCTCCCAA | | |
| | | | AGTGCTGGGATTACAGGAGTGA | | |
| | | | GCCACCATGCCTGGCCAGAAGTGG | | |
| | | | TTACTTCTGTAGACAAAAGAATAA | | |
| | | | TGCTACTTAATCAGGCTTTCTG | | |
| | | | TGTGACAAGAAAGAGAAAGAAAA | | |
| | | | TAAAGAAGTTTCAATTCATCCAAT | | |
| | | | TCTTAATAAGAAATATGTAAATA | | |
| | | | AAATTTTTTAAAATTACACTTCATT | | |
| | | | TTAATGTTGTATCAGTCAAGGTCCC | | |
| | | | TGCAAGAGATGGATGGTATG | | |
| | | | GTACACTCAAACTGGGTAACACAG | | |
| | | | GAGAGTTTTCAGAAAGCAACTAAA | | |
| | | | TCCAAAATACTATCAAGGAATC | | |
| | | | AATATAAAAATTGTTAATATTTTTC | | |
| | | | TCATACTAAATTTTCAAAATATTTT | | |
| | | | GTGTCTATTACATTTACAGC | | |
| | | | ACATCTTAATTAGGACTAGCTGTG | | |
| | | | TGTTCACCTCACATGTGGCTTGTAG | | |
| | | | CTACCATACTGGACAGCACAT | | |
| | | | GTCCAAAAAAATACACGTAAAGTT | | |
| | | | AAAGTTTAAAAGACACAGGAACTA | | |
| | | | AGCCCTCATTGTCTTTCCCTTG | | |
| | | | GGAGGTAGTTTAAAGAGCTATAGA | | |
| | | | TGCTGTAACATTCTTGCTATTATTT | | |
| | | | ATTATATATGACATTATTCCT | | |
| | | | AAAAAAGCTTTTGAGATCCTAGGT | | |
| | | | TGTATTCCTCAGGTTTTGTTGCCTT | | |
| | | | CCCATGAAGATGTGAAGGCAG | | |
| | | | GGATGCCTGTTATTCAGTCCAAGA | | |
| | | | TGCATGACAAGAGACCTTGGGAAA | | |
| | | | GTTTCATCTGGATTTAAAGATT | | |
| | | | AATTCTTGATGCTTACATTCCATAC | | |
| | | | TCAAAATGTAAATTTGAATATTAA | | |
| | | | AATAAAGATGATTTTTTTTTT | | |
| | | | GGAGCTAGTCTTGCTCTGTTGCCCA | | |
| | | | GGCTGGAATGCAGTGGCATGATCA | | |
| | | | TGGCTCACTGCAGCCTCGACC | | |
| | | | TCCCAAGCTCAAGCAAGGCTACAG | | |
| | | | GTGTGCACCTAAGTAGCTAGGACT | | |
| | | | ACAGGTGTGCACCACCATGTCT | | |
| | | | AGCTATTTTTTTTTCTGTAGAGACA | | |
| | | | GGGTTTTCCTATGTTGTCCAGGCTG | | |
| | | | GTCTCGAACTCCTGCCCTCA | | |
| | | | AGCAATCCTCCTGCCTTGGCCTCCC | | |
| | | | AAAGTGTTGAGATTACAGGCGTAA | | |
| | | | GCCACTGCACCTGGCCAAGAT | | |
| | | | GAATATTTTAATAGCTCACAGAAC | | |
| | | | AAAGTTTGCCACATAATGATAAAA | | |
| | | | TTACTATGAAAATATATTCCCT | | |
| | | | TTATTGTCAGTTTAAAAGATGAAC | | |
| | | | TGAGTTTCACCCAAACTGGTCTGG | | |
| | | | CCCCTCTCTGATTCAAATACCA | | |
| | | | ATAGTTGCTCTGATTCAAATTCCAA | | |
| | | | CTGTTAGAACATGACAGCTGCTCA | | |
| | | | TAACTAGCTTTGCTTACTAAC | | |
| | | | CATGTTTCTTTCCATTTGTATTAGG | | |
| | | | TCCTTTACTTTTTATAACAGCCTCA | | |
| | | | AAGTTTCATGAATTGCTGCA | | |
| | | | GTAAACATTGATTTTCATGTTTGTG | | |
| | | | AGTCTGCAAGCCAGCTGGGCAGCT | | |
| | | | CTACTTCAGGTGGTAAGGCTG | | |
| | | | CATCAGACCTATTCCATATACCTCT | | |
| | | | TGTTCTCCTTGTCCAGTGGTTTCTA | | |
| | | | GGGATATGTTCTCATGATGA | | |
| | | | ACCCCGCAGAGGCTCGTGAAAGTG | | |
| | | | AGAGGAAACTAGGATGCCTCTTAA | | |
| | | | GGTCTTGGTCAGGATGGGGTCT | | |
| | | | CCTGTCACTTCTGTCACAGGCTATT | | |
| | | | GTAAGTCATATGAGCAAGCTCAAT | | |
| | | | AAAATATAAACAAGTCAGATA | | |
| | | | AACAGTGGGAGGAATGGCAAAGT | | |
| | | | CATATGGCCAAGGCCATGAGTGAT | | |
| | | | TAATTTTAACACAGGAAAAAAGT | | |
| | | | AAAGCATTAAATGCGATTATTTAA | | |
| | | | TATACAATGTCTTATTAACTGAAAT | | |
| | | | ATAAAATGTGTTTACTGTAAA | | |
| | | | ATATAATCTGTTTATCTCACCAAAG | | |
| | | | AAATATTATCTTTAAAAAATGTCA | | |
| | | | TTACTTCTAAGACATCATCAG | | |
| | | | TCTGCAACTTCTTTCCATAGCCTTA | | |
| | | | ATCAGGATGCTGTGGCAGCTCCCA | | |
| | | | CATTAGCCTCGCATTCTAAAC | | |
| | | | TGGTAGATGTCCTAGGAAACCATA | | |
| | | | CATCTATGTATTTTTCTTATTTTAT | | |
| | | | ACGTTTAGGACAATGTATAGC | | |
| | | | TAATTACCCAACTTTTTATTTGCAT | | |
| | | | ACAAATCTAATACAACTGAACACA | | |
| | | | ATCAGTTTTATCACAGGTATA | | |
| | | | ATGGATTTTTCAATAGTGAGGAGG | | |
| | | | TGCCTCCATGAGCCTTCTCTTTAGA | | |
| | | | AAAGTGGCATTCAAGACTCTT | | |
| | | | CATTTGAAGTGAAGATTGCTATGT | | |
| | | | CTTTTGCATTGCTCTATTTTACATA | | |
| | | | AATTAAGTTATAAATTGACAC | | |
| | | | TATAATCAACTGACACCATGATCA | | |
| | | | GTGATGATGATCACCCTCATCAGC | | |
| | | | ACTAGAGTTGACTTGTTTTTAT | | |
| | | | AACCCCTTTGCATGTATGTTGAATA | | |
| | | | GCAAAGTTCATCAGAGAACATGTA | | |
| | | | TTAGTCAATGGTAAGTAAGAT | | |
| | | | ACTCTCATCTAAGAAATAACATCA | | |
| | | | CCTCTTCTAATCAAGTTCTAAGAA | | |
| | | | GAGAGGGAAGAAAAAGTCTTGG | | |
| | | | GAGCTAGTCAGGGAATAGTGTGTA | | |
| | | | TTTGCAATTACCTAAACTGAACTCT | | |
| | | | ACCATTACTCCTAACCCAGTT | | |
| | | | CCTCCTCCTGTGTTTTACATGATTA | | |
| | | | ATGCCACCGCTGCCTCAATGAACC | | |
| | | | AAGATCAGCTCCATCACTGGG | | |
| | | | ACCTCCCCATTCTGCCTGTGCAATA | | |
| | | | TTTTTCTTTTTTATTTCTCCTTCTAA | | |
| | | | TATTACTGTTATTGCTCCA | | |
| | | | GTAAAGAGCTGTAATATATTTTAC | | |
| | | | CTGGACTGATACCAGGAATGGTGG | | |
| | | | TGTTGCTTCCAATCTGTTGCTG | | |
| | | | CTAGATTAATCTTTGCAAAGCACA | | |
| | | | GGCTTAATTTCATTGCTGCTCAACT | | |
| | | | AAAACCACTGGTGGCTTTCCA | | |
| | | | TTGCCTACAAAATAAAGTCAACCT | | |
| | | | CCCCATCAGACATTCAAGGCTTTC | | |
| | | | AATGATCCATGGCCGCCAGCTC | | |
| | | | TCTCCAGGCTCATATCCCACTCCAC | | |
| | | | TCCTCTGATGTTTCCTACACTACAC | | |
| | | | TACACTATACTACACTACAG | | |
| | | | CCAGGTAGAATGACTGTTCACCCA | | |
| | | | ACACCACTCAGGTTGTCTTCTCAA | | |
| | | | CTTGGAATACTCTTGCACCTTC | | |
| | | | AAAGCTCATTTCAAATGCCCCTTC | | |
| | | | ATTTGTGAAGCCTTCTCCAAATTTC | | |
| | | | CAAGTCAGAATGTCTCTTCCT | | |
| | | | TGTGCTACCACAACCCTTTAACTG | | |
| | | | AGCCTCCATTAGTGCACTGAGACC | | |
| | | | ATTCTGTTCAGTGTCTGGGTGA | | |
| | | | AGCTTCCTGGTGAAAAATATGTTA | | |
| | | | CCTATTTCTTTCTGAAAAGTTGGAT | | |
| | | | TCAGGGATATTATCACGGACC | | |
| | | | TAAGGTAATAGTTCTAGCCAACCT | | |
| | | | CCCTGTCCACTGCCAGGCCGACTA | | |
| | | | CAAACCCTTCTGTTGCTGGCGA | | |
| | | | GCTGGTCCGCACCACTAGTTCTGC | | |
| | | | TTCACTCTATTTATCTCTTGATGTA | | |
| | | | ACCATCTTCTTTCTCCAGGTT | | |
| | | | TTAAGAACCAGCCCAACTCCTGGT | | |
| | | | TCCCTGATGAAGCTTTTATTCCCCT | | |
| | | | AGCCACATGGAACTTTTCCTT | | |
| | | | TTTGGAACATGCCTTTAGTTTCTGT | | |
| | | | GTAGTTTGCCATGCAGCACTTCATT | | |
| | | | CTACACATTATTAAAACAGA | | |
| | | | ATTTTAAGGATTAGAATGAACCTT | | |
| | | | AAAAGATCATGCATCTCAAAATTT | | |
| | | | AATGTACATACAAATTACCCAG | | |
| | | | GGATTTTGTTGAAATAAAAATTAT | | |
| | | | TTAATTTTAATTAATATAAATAATT | | |
| | | | CAGTAGGTCTGGGGTGAGGCC | | |
| | | | TGAGGTTTTACATTTCCAACAAGCT | | |
| | | | GCCAGGTAAAGCCAATACATCTGT | | |
| | | | CCAGGAATCACACTTTGCGTA | | |
| | | | TCAAAGGTCTAGATGACATTATCA | | |
| | | | TTCCAAAGAGTTTCTTTTACAGGCT | | |
| | | | CTCAGATCAGTGTTCATCCAC | | |
| | | | TACCTGACTACTGTCATTCACAGG | | |
| | | | CATTCTGTTCCACACCAGGCCAGC | | |
| | | | TAACGTGGTATTTACAAAGCTC | | |
| | | | ACTCCTCTTATACAACAATCCAAG | | |
| | | | TGTTTCTTTTGTCAGTTGTCTGTGC | | |
| | | | CCCAGGAGATCCCTCTCTGCC | | |
| | | | TTGCCTTGCCCTCTGCCTTTGGAGA | | |
| | | | CCAGCACCTCATACTCAGTGAAGG | | |
| | | | CCTGGAGTGCTTAAGAGGGAT | | |
| | | | TTCTTCCAGCTCTCTTGCCCTGGTC | | |
| | | | TTCAGTGTATTAGATGTATTACCTC | | |
| | | | CATGCTCTCAGTAGAGGCCC | | |
| | | | ATAGGAAAGAGTAGGTAGGTTATG | | |
| | | | CCAGCTCACACGCATCCTTTAAAA | | |
| | | | ATGGTTTAGAAGTTTAGCTGGT | | |
| | | | TTCTTATTACTCCTGTCTATGGATG | | |
| | | | TTTCCTTCTGTCACTCTACTAGGGA | | |
| | | | TGAAACAGCTAATCATGTTC | | |
| | | | AATAGTTACATTTAGATTGGTTTTT | | |
| | | | AAAAACTATGATTGTATTAGTTCG | | |
| | | | TTTCCATGCTGCTGATAAAGA | | |
| | | | CATATCTGAGACTGGAAACAAAAA | | |
| | | | GGGTTTAATTGGACTTACAGTTCC | | |
| | | | ACATGGCTGGGGAGGCCTCAAA | | |
| | | | ATCAGGTGGGAGGCAAAAGGTACT | | |
| | | | TCTTACGTGGTGGCATCAAGAGCA | | |
| | | | AAATGAGGAAGAAGCAAAAGCA | | |
| | | | GAAACTCTTCATAAACCCACCAGA | | |
| | | | TCTTGTGGGACTTATTATCACGAG | | |
| | | | AATAGCACAGAAAAGACTGGCC | | |
| | | | TCCATGATTCAATTACCTCCCACTG | | |
| | | | CGTCCCTCCCACAACATGTGGGAA | | |
| | | | TTCTGGGAGATACAATTCAAG | | |
| | | | TTGAGATTTGGGTGGGGACACAGC | | |
| | | | CAAACCATATCATTCCTCCCTGGG | | |
| | | | CTCCTCCAAATTTCATAATCCT | | |
| | | | CACATTTCAAAACCAATCATTCCTT | | |
| | | | CCCAACAGTTCCCCAAAGTCTTAA | | |
| | | | CTCATTTCAGCATTAACCCAA | | |
| | | | AAGTCCACAGTCCAAAGTCTCATC | | |
| | | | TGAGACAAGGCAAGTCCCTTCCAC | | |
| | | | TTACAAGCCTGTAAAAGCAAGC | | |
| | | | TAGTTACCTCCTAGATACAATGGG | | |
| | | | GGGTACAGGTATTGGGTAAATACA | | |
| | | | GCTGTTCCAAATGAGAGAAATT | | |
| | | | GGCCAAAACAAAGGGGTTACAGG | | |
| | | | GTCCATGCAAGTCTGAAATCCAGT | | |
| | | | GGGGCACTCAAATTTTAAAGCTC | | |
| | | | CATAATGATCTCCTTTGACTCCATG | | |
| | | | TCTCACATTCAGGTCATGCTGATGC | | |
| | | | AAGAGATAGGTTCCCATGGT | | |
| | | | CTTGTGCAGCTCCGCCCCTGTGGCT | | |
| | | | TTGCAGAGTACAGCCTCCCTCCTG | | |
| | | | GCTGCTTTCTCAGGCTGATGT | | |
| | | | TGAGTGTCTGTAGCTTTTCCAGGCA | | |
| | | | CAAGATGCAAGTTGGTGGTTGATC | | |
| | | | TACCATTCTGGGGTCTACCAT | | |
| | | | TCTGGGGTCTACCGTTCTGGGACT | | |
| | | | GTGGCCTTCTTCTCACAGCTCCACT | | |
| | | | AGGCAGTGCCCCAACAGGGAC | | |
| | | | TCTGTGTGGGGGCTCTGCCCCACA | | |
| | | | TTTCCCTTCCACACTGCCCTAGGAG | | |
| | | | AGGTTCCCCATGAGGGCTCTG | | |
| | | | CCCCTGCAGCAAACTTTTGCCTGG | | |
| | | | ACATCCAGGTGTTTCCATATATATT | | |
| | | | CTGAAATCTAGGCAGAGGTTC | | |
| | | | CCAAATCTCAATTCTTGACATCTCT | | |
| | | | GCACCCACAGGCTCAACATCACAT | | |
| | | | GGAAGCTGCCAATGCTTGGGG | | |
| | | | CCTCTACCCTCTGAAGCCACAGCC | | |
| | | | CAAGCTCTATGTTGGCTCCTTTCAG | | |
| | | | CCATGGCTGGAGCAGCTGGGA | | |
| | | | CACAGGGCACCAAGTCCCTAGGCT | | |
| | | | GCACACAGCACAGAGACCCTGGGC | | |
| | | | CCAGCCCACAAAACCACTTTTT | | |
| | | | CCTCCTGGGCCTCTGGGCCTGTGA | | |
| | | | TGGGAGGGGCTGCCATGAAGGTCT | | |
| | | | CTGACATGACCTGGAGACATTT | | |
| | | | TCCCCATGGTCTTGGGGATTAACA | | |
| | | | TTAGGCTCCTTGCTGCTTATGCAAA | | |
| | | | TTTCTGCAGCCAGCTTGAATT | | |
| | | | TCTCCTTAAAAAAAATGGGTTTTTC | | |
| | | | TTTTCTACTGCATCATCAGGCTGCA | | |
| | | | GATTTTCCACATTTATGCTC | | |
| | | | TTGTTTCCCTTTTAAAACAGAATGT | | |
| | | | TTTTAACAGCACCCAAGTCACCTTT | | |
| | | | TGAATGCTTTGCTGCTTAGA | | |
| | | | AATTTATTCCACCAGATACCCTAA | | |
| | | | GTCATCTCTCTCAAGCTCTAAGTTC | | |
| | | | CACAAATCTCTAGGGCAAGGG | | |
| | | | TGAAATGCTGCCAGTCTCCTTGCTA | | |
| | | | AAACATAACAAGGGTCACCTTTAC | | |
| | | | TTCAGTTCCCAACAAGGTCTT | | |
| | | | CATCTCCATCTGAGACCACCTCAG | | |
| | | | CCTGGACCTTATTGTTCATATCACT | | |
| | | | ATCAGTATTTTTGTCAATGCC | | |
| | | | ATTCACAGTCTCTAGGAGGTTCCA | | |
| | | | AACTTTCCTACATTTTCCTATCTTC | | |
| | | | TTCTGAGCCCTCCAGATTATT | | |
| | | | TCAACACCCAGTTCCAAAGTTGCT | | |
| | | | TCCACATTTTCGGGTATCTTTTCAG | | |
| | | | CAATGCCCCACTCTACTGGTA | | |
| | | | CTATTAGTCCATTTTCATGCTGCTG | | |
| | | | ATAAAGACATACCTGAGACTGGGA | | |
| | | | ACAAAAAGAGGTTTAATTGGA | | |
| | | | CTTATAGTTCCACCTGGCTGGGGA | | |
| | | | GGCCTCAGAATCATGGCAGGAGGT | | |
| | | | GAAAGGCATTTCTTACACGGCA | | |
| | | | GCAGCAAGAGAAAAATGAAGAAG | | |
| | | | CAGCAAAAGCAGAAACCCCTGATA | | |
| | | | AAACCATCAGATCTCGTGAGACT | | |
| | | | TATTCACTATCACAAGAATAGCAT | | |
| | | | GGGAAAGACCAGCCCCCTTGATTC | | |
| | | | AATTACCTCCCCCTGGGTCCTG | | |
| | | | TGGGAATTCTGGAAGGTACAATTC | | |
| | | | AAGTTGAGATTTGGGTGGGGACAC | | |
| | | | AGCCAAACCATATCAATGATTT | | |
| | | | TGTACTTTAACCAGCTGAATGGAA | | |
| | | | GTACAATCTCTTGCTATATGACAC | | |
| | | | AATAATTATTTGCAAAATGAGT | | |
| | | | AAACATATCATAAGGAAATTATTT | | |
| | | | TTACAAGGTTTGAAACCTGAAATG | | |
| | | | CAGTCTATTATCATACATAACT | | |
| | | | AAAAATAGAGCCTCAATAAACAGA | | |
| | | | TTCCCAGTTTTGAAAATGCAACATT | | |
| | | | TGTACTCCACATTGTCAGTTT | | |
| | | | TCTTAGGTATATTTATAAATACTCC | | |
| | | | TATAAAAATGTAAAGAAACACATA | | |
| | | | ATGTAGATTGCTAATTTTATA | | |
| | | | ATAACACAAGTTGATTTTGACATC | | |
| | | | CAACTTATTAATTATGAAATGACTT | | |
| | | | TTGGCCTAGTAACAATGAAAA | | |
| | | | TGGGGGCAAATACAGATAAATGGT | | |
| | | | AATTCTTAGAATGAACTACTCAGC | | |
| | | | ACCAATTCTAAGTTTTTCTTGA | | |
| | | | TGGTAAATCATAATGTTCCCTTTCT | | |
| | | | CCTCGGTTCTGCAATCTATAGGCAT | | |
| | | | ACCATAATTGTAATCAATAG | | |
| | | | CTTAAAAATATGTCTCTCTGTCCTA | | |
| | | | TTCTGTATCTGTATCTCTTGGATTT | | |
| | | | TTACCTTTGCAATACTCAAC | | |
| | | | TGAACCATCTTCTTGGAGTACTCAT | | |
| | | | GAAGATGGAAGTTCTACATGGAGAA | | |
| | | | TACAGGATGAATCCACTCTGT | | |
| | | | CTCCTGCAGTGAAGTCTGTTTGAA | | |
| | | | GGATGTATTTGGCTGTCTTCTGGAC | | |
| | | | AGGCCATTCTAATAACAGAAA | | |
| | | | CAAACAAGTTATTTTAAAACTTATT | | |
| | | | GGAATATTCAAATATTAACCAAAG | | |
| | | | TAGAAAAATATAATACACATC | | |
| | | | CATGTGCCCATCACAGAACTTCAC | | |
| | | | TGATTATCATCATTTAGCCAGTCTT | | |
| | | | GAAGAAGCAAGTGCTAATTAC | | |
| | | | AATCACAAATGAAACAAGATTCAG | | |
| | | | ACTTCATGAAGAGCACTGCGCTAT | | |
| | | | AATAAAAGAAGAAATGAGCACA | | |
| | | | TACATTCTTTTACTGACAGTCAAAT | | |
| | | | GGTGAAGGTGGGCAGAATCATTAT | | |
| | | | GTGATGCAACATCGCAAAAGT | | |
| | | | ATACAGACAGTGCATCCAGAGGAA | | |
| | | | GGCACCTTGCTGAATGACTAGAAT | | |
| | | | GGAAGTAGGAGACATTTTGCAG | | |
| | | | GCCCCCTTCATCCTGCAGGGAGAA | | |
| | | | CCAGAACCACAGCAGCTCTATTTG | | |
| | | | CCTATTCCTCTTTAAATTACAA | | |
| | | | AGTTAAAATTTGGGAGTAGTAGAA | | |
| | | | AATCAATTGGTTATCTTATAGAGTC | | |
| | | | TCCTAGAATATTTCATTGCCA | | |
| | | | TTGAGAAGGTGGAAAATGCAAATT | | |
| | | | ATATACTTTAAAATGTAATTTTTGC | | |
| | | | TTTTCACATATGCTTAAAGCC | | |
| | | | TAAAACCTCTTAATAAACTTCTTCT | | |
| | | | GAAATATA (SEQ ID NO: 33) | | |
| | | NM_001098175.2 | ATTCTGCAGTCTCCTGTGTACGTGT | NP_001091645.1 | MKGTKDLTSQQKESNV |
| | | | AAAATTATGATCAAATAAATTTGT | | KTFCSKNTLAILGFSSIIA |
| | | | ATGCCTTTTCTCCTATTAACC | | VIALLAVGL |
| | | | TGCCTTTTTTGTCAGCGATTGTCAG | | TQNKALPENVKYGIVLD |
| | | | TGAAACTTCAGAGGGCAAAGGGG | | AGSSHTSLYTYKWPAEK |
| | | | AAGTTTTCCTTGGCCCCTCCAG | | ENDTGVVHQVEECRVK |
| | | | TTTTGGTGCTGTGAACAGGATACC | | GPGISKFV |
| | | | AAAGCTGCTCTGTTCTTCTGGAAG | | QKVNEIGIYLIDCMERA |
| | | | CTGCAATGAAGGCAACCAAGGA | | REVIPRSQHQETPVYLG |
| | | | CCTGACAAGCCAGCAGAAGGAGTC | | ATAGMRLLRMESEELA |
| | | | TAACGTGAAGACATTTTGCTCCAA | | DRVLDVVE |
| | | | GAATATCCTAGCCATCCTTGGC | | RSLSNYPFDFQGARIITG |
| | | | TTCTCCTCTATCATAGCTGTGATAG | | QEEGAYGWITINYLLGK |
| | | | CTTTGCTTGCTGTGGGGTTGACCCA | | FSQKTRWFSIVPYETNN |
| | | | GAACAAAGCATTGCCAGAAA | | QETFGA |
| | | | ACGTTAAGTATGGGATTGTGCTGG | | LDLGGASTQVTFVPQN |
| | | | ATGCGGGTTCTTCTCACACAAGTTT | | QTIESPDNALQFRLYGK |
| | | | ATACATCTATAAGTGGCCAGC | | DYNVYTHSFLCYGKDQ |
| | | | AGAAAAGGAGAATGACACAGGCG | | ALWQKLAKD |
| | | | TGGTGCATCAAGTAGAAGAATGCA | | IQVASNEILRDPCFHPGY |
| | | | GGGTTAAAGGTCCTGGAATCTCA | | KKVVNVSDLYKTPCTK |
| | | | AAATTTGTTCAGAAAGTAAATGAA | | RFEMTLPFQQFEIQGIGN |
| | | | ATAGGCATTTACCTGACTGATTGC | | YQQCHQ |
| | | | ATGGAAAGAGCTAGGGAAGTGA | | SILELFNTSYCPYSQCAF |
| | | | TTCCAAGGTCCCAGCACCAAGAGA | | NGIFLPPLQGDFGAFSAF |
| | | | CACCCGTTTACCTGGGAGCCACGG | | YFVMKFLNLTSEKVSQE |
| | | | CAGGCATGCGGTTGCTCAGGAT | | KVTEM |
| | | | GGAAAGTGAAGAGTTGGCAGACA | | MKKFCAQPWEEIKTSY |
| | | | GGGTTCTGGATGTGGTGGAGAGGA | | AGVKEKYLSEYCFSGT |
| | | | GCCTCAGCAACTACCCCTTTGAC | | YILSLLLQGYHFTADSW |
| | | | TTCCAGGGTGCCAGGATCATTACT | | EHIHFIGKI |
| | | | GGCCAAGAGGAAGGTGCCTATGGC | | QGSDAGWTLGYMLNLT |
| | | | TGGATTACTATCAACTATCTGC | | NMIPAEQPLSTPLSHSTY |
| | | | TGGGCAAATTCAGTCAGAAAACAA | | VFLMVLFSLVLFTVAIIG |
| | | | GGTGGTTCAGCATAGTCCCATATG | | LLIFHK |
| | | | AAACCAATAATCAGGAAACCTT | | PSYFWKDMV (SEQ ID |
| | | | TGGAGCTTTGGACCTTGGGGGAGC | | NO: 36) |
| | | | CTCTACACAAGTCACTTTTGTACCC | | |
| | | | CAAAACCAGACTATCGAGTCC | | |
| | | | CCAGATAATGCTCTGCAATTTCGC | | |
| | | | CTCTATGGCAAGGACTACAATGTC | | |
| | | | TACACACATAGCTTCTTGTGCT | | |
| | | | ATGGGAAGGATCAGGCACTCTGGC | | |
| | | | AGAAACTGGCCAAGGACATTCAGG | | |
| | | | TTGCAAGTAATGAAATTCTCAG | | |
| | | | GGACCCATGCTTTCATCCTGGATAT | | |
| | | | AAGAAGGTAGTGAACGTAAGTGAC | | |
| | | | CTTTACAAGACCCCCTGCACC | | |
| | | | AAGAGATTTGAGATGACTCTTCCA | | |
| | | | TTCCAGCAGTTTGAAATCCAGGGT | | |
| | | | ATTGGAAACTATCAACAATGCC | | |
| | | | ATCAAAGCATCCTGGAGCTCTTCA | | |
| | | | ACACCAGTTACTGCCCTTACTCCC | | |
| | | | AGTGTGCCTTCAATGGGATTTT | | |
| | | | CTTGCCACCACTCCAGGGGGATTT | | |
| | | | TGGGGCATTTTCAGCTTTTTACTTT | | |
| | | | GTGATGAAGTTTTTAAACTTG | | |
| | | | ACATCAGAGAAAGTCTCTCAGGAA | | |
| | | | AAGGTGACTGAGATGATGAAAAA | | |
| | | | GTTCTGTGCTCAGCCTTGGGAGG | | |
| | | | AGATAAAAACATCTTACGCTGGAG | | |
| | | | TAAAGGAGAAGTACCTGAGTGAAT | | |
| | | | ACTGCTTTTCTGGTACCTACAT | | |
| | | | TCTCTCCCTCCTTCTGCAAGGCTAT | | |
| | | | CATTTCACAGCTGATTCCTGGGAG | | |
| | | | CACATCCATTTCATTGGCAAG | | |
| | | | ATCCAGGGCAGCGACGCCGGCTGG | | |
| | | | ACTTTGGGCTACATGCTGAACCTG | | |
| | | | ACCAACATGATCCCAGCTGAGC | | |
| | | | AACCATTGTCCACACCTCTCTCCCA | | |
| | | | CTCCACCTATGTCTTCCTCATGGTT | | |
| | | | CTATTCTCCCTGGTCCTTTT | | |
| | | | CACAGTGGCCATCATAGGCTTGCT | | |
| | | | TATCTTTCACAAGCCTTCATATTTC | | |
| | | | TGGAAAGATATGGTATAGCAA | | |
| | | | AAGCAGCTGAAATATGCTGGCTGG | | |
| | | | AGTGAGGAAAAAAATCGTCCAGG | | |
| | | | GAGCATTTTCCTCCATCGCAGTG | | |
| | | | TTCAAGGCCATCCTTCCCTGTCTGC | | |
| | | | CAGGGCCAGTCTTGACGAGTGTGA | | |
| | | | AGCTTCCTTGGCTTTTACTGA | | |
| | | | AGCCTTTCTTTTGGAGGTATTCAAT | | |
| | | | ATCCTTTGCCTCAAGGACTTCGCC | | |
| | | | AGATACTGTCTCTTTCATGAG | | |
| | | | TTTTTCCCAGCTACACCTTTCTCCT | | |
| | | | TTGTACTTTGTGCTTGTATAGGTTT | | |
| | | | TAAAGACCTGACACCTTTCA | | |
| | | | TAATCTTTGCTTTATAAAAGAACA | | |
| | | | ATATTGACTTTGTCTAGAAGAACT | | |
| | | | GAGAGTCTTGAGTCCTGTGATA | | |
| | | | GGAGGCTGAGCTGGCTGAAAGAA | | |
| | | | GAATCTCAGGAACTGGTTCAGTTG | | |
| | | | TACTCTTTAAGAACCCCTTTCTC | | |
| | | | TCTCCTGTTTGCCATCCATTAAGAA | | |
| | | | AGCCATATGATGCCTTTGGAGAAG | | |
| | | | GCAGACACACATTCCATTCCC | | |
| | | | AGCCTGCTCTGTGGGTAGGAGAAT | | |
| | | | TTTCTACAGTAGGCAAATATGTGC | | |
| | | | TAAAGCCAAAGAGTTTTATAAG | | |
| | | | GAAATATATGTGCTCATGCAGTCA | | |
| | | | ATACAGTTCTCAATCCCACCCAAA | | |
| | | | GCAGGTATGTCAATAAATCACA | | |
| | | | TATTCCTAGGTGATACCCAAATGC | | |
| | | | TACAGAGTGGAACACTCAGACCTG | | |
| | | | AGATTTGCAAAAAGCAGATGTA | | |
| | | | AATATATGCATTCAAACATCAGGG | | |
| | | | CTTACTATGAGGTAGGTGGTATAT | | |
| | | | ACATGTCACAAATAAAAATACA | | |
| | | | GTTACAACTCAGGGTCACAAAAAA | | |
| | | | TGCATCTTCCAATGCATATTTTTAT | | |
| | | | TATGGTAAAATATACATAAAT | | |
| | | | ATAATTCACCATTTTAACATTTAAT | | |
| | | | TCATATTAAATACGTACAAATCAG | | |
| | | | TGACATTTAGTACATTCACAG | | |
| | | | TGTTGTGCCACCATCACCACTATTT | | |
| | | | AGTTCCAGAACATTTGCATCATCA | | |
| | | | ATACATTGTCTAGAGACAAGA | | |
| | | | CTATCCTGGGTAGGCAGAAACCAT | | |
| | | | AGATCTTTTGTGTTTACAGCTATGG | | |
| | | | AAACCAACTGTACCATAAAGA | | |
| | | | TAGTTCACTGAGTTTTAAAGCCAA | | |
| | | | GCCACATCTTATTTTTCCAAGGTTT | | |
| | | | AATTTAGTGAGAGGGCAGCAT | | |
| | | | TAGTGTGGAGTGGCATGCTTTTGC | | |
| | | | CCTATCGTGGAATTTACACATCAG | | |
| | | | AATGTGCAGGATCCAAGTCTGA | | |
| | | | AAGTGTTGCCACCCGTCACACAAC | | |
| | | | ATGGGCTTTGTTTGCTTATTCCATG | | |
| | | | AAGCAGCAGCTATAGACCTTA | | |
| | | | CCATGGAAACATGAAGAGACCCTG | | |
| | | | CACCCCTTTCCTTAAGGATTGCTGC | | |
| | | | AAGAGTTACCTGTTGAGCAGG | | |
| | | | ATTGACTGGTGATGTTTCATTCTGA | | |
| | | | CCTTGTCCCAAGCTCTCCATCTCTA | | |
| | | | GATCTGGGGACTGACTGTTG | | |
| | | | AGCTGATGGGGAAAGAAAAGCTCT | | |
| | | | CACACAAACCGGAAGCCAAATGTC | | |
| | | | CCCTATCTCTTGAATGATCAAG | | |
| | | | TCACTTTTGACAACATCCAGGTGA | | |
| | | | ATATAAAAACTTAATAAAGCTGTG | | |
| | | | GAAAGGAACTCTTAATCTTCTT | | |
| | | | TTCTGCTACTTAGGTTAAATTCACT | | |
| | | | AGATCTTGATTAGGAATCAAAATT | | |
| | | | CGAATTGGGACATGTTCAAAT | | |
| | | | TCTTTCTTGTGGTAGTTGCCTATAC | | |
| | | | TGTCATCGCTGCTGTTGGTTGAGCA | | |
| | | | TTTCTGGTGTACCACGCTGT | | |
| | | | GTGCTCAAGGGTATTACATTCATCT | | |
| | | | TCTCATTTAATCCTCACAACAATCT | | |
| | | | GAAGAAGGTAGGTATTACAA | | |
| | | | TTCCCACTTCATAGAAACAGAAAC | | |
| | | | TGAGGTTCAGAGAGGTTAAGTCAT | | |
| | | | TTGCCCAAATGGCTGAGCCAAA | | |
| | | | GCCTACCATGTACCTAACCTTTATT | | |
| | | | TTCTTTCCCGAACATACCAGGCTGT | | |
| | | | CTCCTCATAACTTCCAAGCA | | |
| | | | TGCACTTAAAACTCCACATGAATA | | |
| | | | CAAGGTTCATGGGACTTGGTATTC | | |
| | | | ATAGAAAGGGAGGCAGAAACCT | | |
| | | | GGTCTGTTCCTGATAGGCTTGTAAT | | |
| | | | TTAATATCATTCTGTTCATGTGCTT | | |
| | | | TGGATGGAAGCACATCTGGC | | |
| | | | ATATGATGCTAATCAGTGGTTCCC | | |
| | | | ATACCCCTGGCTTCCTAATTTTAAT | | |
| | | | GTTTGCTCACAGCATACTAGA | | |
| | | | TTGACATCAAATAGTGGCCGATGA | | |
| | | | TGATGAAAATAAAGGTCAAATAAG | | |
| | | | TTGAGCCAATAACAGCCGCTTT | | |
| | | | TTTCCTTCTGTCTGCGTATACAAAG | | |
| | | | CACTGTCATGCACACAATCTATTCT | | |
| | | | GACCCTCACAACAACCCATA | | |
| | | | AGGGTGTAAATAGTATTTCCATTTT | | |
| | | | ACAAATGAGGATCACACAAACTAC | | |
| | | | TACATGGCAGAGCAGATACTC | | |
| | | | CAACTCATGTCTTCTGGTTGAAGCC | | |
| | | | TATTGCTTTTTCTTTTCTAAACACT | | |
| | | | TTCCCTCACCAAGTTGGAAT | | |
| | | | TAGACTTCACAAGTCTCCTTCAGA | | |
| | | | CAACACAAATCTTTTCTTATTCCAT | | |
| | | | TCCTGTTTGGTTGCCTACGTC | | |
| | | | CAATCTCCCCCTCCCCAGAGATGC | | |
| | | | CAAAAAAAAAATCCTTTAAGGTAT | | |
| | | | TTGGGAGCCAAACTCAACTTGT | | |
| | | | TAAAATCTCAAATTATGGAGACAA | | |
| | | | TCAGCAGACACAACCTAACCCCAA | | |
| | | | TTATTTTGGCAGGAAGGTTGGT | | |
| | | | TTAGAGGCAGATCCAGCAATCTGC | | |
| | | | TTTGGGCCACTCTGGGTGGGGTAG | | |
| | | | CTGAAATAAGATTGGTCACTCT | | |
| | | | TAACTAATTTTAATATTGGATTGGC | | |
| | | | CATTGGTTATCACTGATTACCATTC | | |
| | | | TCCCCTGGATTTTCACCCAG | | |
| | | | GACTCAAAACTTGGTTCTGCTAAC | | |
| | | | CCTGTTCCTTTATGAGGAACCTTTT | | |
| | | | AAAGATTCCTTTATAAGGTGG | | |
| | | | GAGTTTTTTTTCTATGAACCTATAG | | |
| | | | GGGAGAAAAAAGATCAGCAGAAG | | |
| | | | TCATTACTTTTTTTTTTTTTTT | | |
| | | | TTTTTTTTTTTGAGAGAGAGTCTCA | | |
| | | | CTCCATTGCCCAGGCTGGAGTGCA | | |
| | | | GTGGTGCTATCTCGGCTCACT | | |
| | | | GCAACCTCCGCCTCCTGGGTTCAA | | |
| | | | CCAATTCTCCTGCCTCAGCCTCCCG | | |
| | | | AGTAGCTGGGATTGCAGGTGC | | |
| | | | CCACCACCACACCCGGCTAATTTT | | |
| | | | TGTATTTTTAGTAAAGACAGGGTTT | | |
| | | | CACCATGTTGGCCAGGCTGGT | | |
| | | | CTCCAACTCCCAATCTCAGGTGAT | | |
| | | | CCTATTGCCTCGGGCTCCCAAAGT | | |
| | | | GCTGGGATTACAGGAGTGAGCC | | |
| | | | ACCATGCCTGGCCAGAAGTGGTTA | | |
| | | | CTTCTGTAGACAAAAGAATAATCC | | |
| | | | TACTTAATCAGGCTTTCTGTGT | | |
| | | | GACAAGAAAGAGAAAGAAAATAA | | |
| | | | AGAAGTTTCAATTCATCCAATTCTT | | |
| | | | AATAAGAAATATGTAAATAAAA | | |
| | | | TTTTTTAAAATTACACTTCATTTTA | | |
| | | | ATGTTGTATCAGTCAAGGTCCCTG | | |
| | | | CAAGAGATGGATGGTATGGTA | | |
| | | | CACTCAAACTGGGTAACACAGGAG | | |
| | | | AGTTTTCAGAAAGCAACTAAATCC | | |
| | | | AAAATACTATCAAGGAATCAAT | | |
| | | | ATAAAAATTGTTAATATTTTTCTCA | | |
| | | | TACTAAATTTTCAAAATATTTTGTG | | |
| | | | TCTATTACATTTACAGCACA | | |
| | | | TCTTAATTAGGACTAGCTGTGTGTT | | |
| | | | CACCTCACATGTGGCTTGTAGCTA | | |
| | | | CCATACTGGACAGCACATGTC | | |
| | | | CAAAAAAATACACGTAAAGTTAAA | | |
| | | | GTTTAAAACACACAGGAACTAAGC | | |
| | | | CCTCATTGTCTTTCCCTTGGGA | | |
| | | | GGTAGTTTAAAGAGCTATAGATGC | | |
| | | | TGTAACATTCTTGCTATTATTTATT | | |
| | | | ATATATGACATTATTCCTAAA | | |
| | | | AAAGCTTTTGAGATCCTAGGTTGT | | |
| | | | ATTCCTCAGGTTTTGTTGCCTTCCC | | |
| | | | ATGAAGATGTGAAGGCAGGGA | | |
| | | | TGCCTGTTATTCAGTCCAAGATGC | | |
| | | | ATGACAAGAGACCTTGGGAAAGTT | | |
| | | | TCATCTGGATTTAAAGATTAAT | | |
| | | | TCTTGATGCTTACATTCCATACTCA | | |
| | | | AAATGTAAATTTGAATATTAAAAT | | |
| | | | AAAGATGATTTTTTTTTTGGA | | |
| | | | GCTAGTCTTGCTCTGTTGCCCAGGC | | |
| | | | TGGAATGCAGTGGCATCATCATGG | | |
| | | | CTCACTGCAGCCTCGACCTCC | | |
| | | | CAAGCTCAAGCAAGGCTACAGGTG | | |
| | | | TGCACCTAAGTAGCTAGGACTACA | | |
| | | | CGTGTGCACCACCATGTCTAGC | | |
| | | | TATTTTTTTTTCTGTAGAGACAGGG | | |
| | | | TTTTCCTATGTTGTCCAGGCTGGTC | | |
| | | | TCGAACTCCTGCCCTCAAGC | | |
| | | | AATCCTCCTGCCTTGGCCTCCCAA | | |
| | | | AGTGTTGAGATTACAGGCGTAAGC | | |
| | | | CACTGCACCTGGCCAAGATGAA | | |
| | | | TATTTTAATAGCTCACAGAACAAA | | |
| | | | GTTTGCCACATAATCATAAAATTA | | |
| | | | CTATGAAAATATATTCCCTTTA | | |
| | | | TTGTCAGTTTAAAACATGAACTGA | | |
| | | | GTTTCACCCAAACTGGTCTGGCCC | | |
| | | | CTCTCTGATTCAAATACCAATA | | |
| | | | GTTGCTCTGATTCAAATTCCAACTG | | |
| | | | TTAGAACATGACAGCTGCTCATAA | | |
| | | | CTAGCTTTGCTTACTAACCAT | | |
| | | | GTTTCTTTCCATTTGTATTAGGTCC | | |
| | | | TTTACTTTTTATAACAGCCTCAAAG | | |
| | | | TTTCATGAATTGCTGCAGTA | | |
| | | | AACATTGATTTTCATGTTTGTGAGT | | |
| | | | CTGCAAGCCAGCTGGGCAGCTCTA | | |
| | | | CTTCAGGTGGTAAGGGTGGAT | | |
| | | | CAGACCTATTCCATATACCTCTTGT | | |
| | | | TCTCCTTGTCCAGTGGTTTCTAGGG | | |
| | | | ATATGTTCTCATCATGAACC | | |
| | | | CCGCAGAGGCTCGTGAAAGTGAGA | | |
| | | | GGAAACTAGGATGCCTCTTAAGGT | | |
| | | | CTTGGTCAGGATGGGGTCTCCT | | |
| | | | GTCACTTCTGTCACAGGCTATTGTA | | |
| | | | AGTCATATGAGCAAGCTCAATAAA | | |
| | | | ATATAAACAACTCAGATAAAC | | |
| | | | AGTGGGAGGAATGGCAAAGTCATA | | |
| | | | TGGCCAAGCCCATGAGTGATTAAT | | |
| | | | TTTAACACAGGAAAAAAGTAAA | | |
| | | | GCATTAAATGCGATTATTTAATAT | | |
| | | | ACAATGTCTTATTAACTGAAATAT | | |
| | | | AAAATGTGTTTACTGTAAAATA | | |
| | | | TAATCTGTTTATCTCACCAAAGAA | | |
| | | | ATATTATCTTTAAAAAATGTCATTA | | |
| | | | CTTCTAAGACATCATCAGTCT | | |
| | | | GCAACTTCTTTCCATAGCCTTAATC | | |
| | | | AGGATGCTGTGGCAGCTCCCACAT | | |
| | | | TAGCCTCGCATTCTAAACTGG | | |
| | | | TAGATGTCCTAGGAAACCATACAT | | |
| | | | CTATGTATTTTTCTTATTTTATACG | | |
| | | | TTTAGGACAATGTATAGCTAA | | |
| | | | TTACCCAACTTTTTATTTGCATACA | | |
| | | | AATCTAATACAACTGAACACAATC | | |
| | | | AGTTTTATCACAGGTATAATG | | |
| | | | GATTTTTCAATAGTGAGGAGGTGC | | |
| | | | CTCCATGAGCCTTCTCTTTAGAAAA | | |
| | | | GTGGCATTCAAGACTCTTCAT | | |
| | | | TTGAAGTGAAGATTGCTATGTCTTT | | |
| | | | TGCATTGCTCTATTTTACATAAATT | | |
| | | | AAGTTATAAATTGACACTAT | | |
| | | | AATCAACTGACACCATGATCAGTG | | |
| | | | ATGATGATCACCCTCATCAGCACT | | |
| | | | AGAGTTGACTTGTTTTTATAAC | | |
| | | | CCCTTTGCATGTATGTTGAATAGCA | | |
| | | | AAGTTCATCAGAGAACATGTATTA | | |
| | | | CTCAATGGTAAGTAAGATACT | | |
| | | | CTCATCTAAGAAATAACATCACCT | | |
| | | | CTTCTAATGAAGTTCTAAGAAGAG | | |
| | | | AGGGAAGAAAAAGTCTTGGGAG | | |
| | | | CTAGTCAGGGAATAGTGTGTATTT | | |
| | | | GCAATTACCTAAACTGAACTCTAC | | |
| | | | CATTACTCCTAACCCAGTTCCT | | |
| | | | CCTCCTGTGTTTTACATGATTAATG | | |
| | | | CCACCCCTGCCTCAATGAACCAAG | | |
| | | | ATCAGCTCCATCACTGGGACC | | |
| | | | TCCCCATTCTGCCTGTGCAATATTT | | |
| | | | TTCTTTTTTATTTCTCCTTCTAATAT | | |
| | | | TACTGTTATTGCTCCAGTA | | |
| | | | AAGAGCTGTAATATATTTTACCTG | | |
| | | | GACTGATACCAGGAATGGTGGTGT | | |
| | | | TGCTTCCAATCTGTTGCTGCTA | | |
| | | | GATTAATCTTTGCAAAGCACAGGC | | |
| | | | TTAATTTCATTGCTGCTCAACTAAA | | |
| | | | ACCACTGGTGGCTTTCCATTG | | |
| | | | CCTACAAAATAAAGTCAACCTCCC | | |
| | | | CATCAGACATTCAAGGCTTTCAAT | | |
| | | | GATCCATGGCCGCCAGCTCTCT | | |
| | | | CCAGGCTCATATCCCACTCCACTC | | |
| | | | CTCTGATGTTTCCTACACTACACTA | | |
| | | | CACTATACTACACTACAGCCA | | |
| | | | GGTAGAATGACTGTTCACCCAACA | | |
| | | | CCACTCAGGTTGTCTTCTCAACTTG | | |
| | | | GAATACTCTTGCACCTTCAAA | | |
| | | | GCTCATTTCAAATGCCCCTTCATTT | | |
| | | | GTGAAGCCTTCTCCAAATTTCCAA | | |
| | | | GTCAGAATGTCTCTTCCTTGT | | |
| | | | GCTACCACAACCCTTTAACTGAGC | | |
| | | | CTCCATTAGTGCACTGAGACCATT | | |
| | | | CTGTTCAGTGTCTGCGTGAAGC | | |
| | | | TTCCTGGTGAAAAATATGTTACCT | | |
| | | | ATTTCTTTCTGAAAAGTTGGATTCA | | |
| | | | GGGATATTATCACGGACCTAA | | |
| | | | GGTAATAGTTCTAGCCAACCTCCC | | |
| | | | TGTCCACTGCCAGGCCGACTACAA | | |
| | | | ACCCTTCTGTTGCTGGCGAGCT | | |
| | | | GGTCCGCACCACTAGTTCTGCTTC | | |
| | | | ACTCTATTTATCTCTTGATGTAACC | | |
| | | | ATCTTCTTTCTCCAGGTTTTA | | |
| | | | AGAACCAGCCCAACTCCTGGTTCC | | |
| | | | CTGATGAAGCTTTTATTCCCCTAGC | | |
| | | | CACATGGAACTTTTCCTTTTT | | |
| | | | GGAACATGCCTTTAGTTTCTGTGTA | | |
| | | | GTTTGCCATGCAGCACTTCATTGTA | | |
| | | | CACATTATTAAAACAGAATT | | |
| | | | TTAAGGATTAGAATGAACCTTAAA | | |
| | | | AGATCATGCATCTCAAAATTTAAT | | |
| | | | GTACATACAAATTACCCAGGGA | | |
| | | | TTTTGTTGAAATAAAAATTATTTAA | | |
| | | | TTTTAATTAATATAAATAATTCAGT | | |
| | | | AGGTCTGGGGTGAGGCCTGA | | |
| | | | GGTTTTACATTTCCAACAAGCTGCC | | |
| | | | AGGTAAAGCCAATACATCTGTCCA | | |
| | | | GGAATCACACTTTGCGTATCA | | |
| | | | AAGGTCTAGATGACATTATCATTC | | |
| | | | CAAAGAGTTTCTTTTACAGGCTCTC | | |
| | | | AGATCAGTGTTCATCCACTAC | | |
| | | | CTGACTACTGTCATTCACAGGCATT | | |
| | | | CTGTTCCACAGCAGGCCAGCTAAC | | |
| | | | GTGGTATTTACAAAGCTCACT | | |
| | | | CCTCTTATACAACAATCCAAGTGTT | | |
| | | | TCTTTTGTCAGTTGTCTGTGCCCCA | | |
| | | | GGAGATCCCTCTCTGCCTTG | | |
| | | | CCTTGCCCTCTGCCTTTGGAGACCA | | |
| | | | GCACCTCATACTCAGTGAAGGCCT | | |
| | | | GGAGTGCTTAAGAGGGATTTC | | |
| | | | TTCCAGCTCTCTTGCCCTGGTCTTC | | |
| | | | AGTGTATTAGATGTATTACCTCCAT | | |
| | | | GCTCTCAGTAGAGGCCCATA | | |
| | | | GGAAAGAGTAGGTAGGTTATGCCA | | |
| | | | CCTCACACGCATCCTTTAAAAATG | | |
| | | | GTTTAGAAGTTTAGCTGGTTTC | | |
| | | | TTATTACTCCTGTCTATGGATGTTT | | |
| | | | CCTTCTGTCACTCTACTAGGGATGA | | |
| | | | AACAGCTAATCATGTTCAAT | | |
| | | | AGTTACATTTAGATTGGTTTTTAAA | | |
| | | | AACTATGATTGTATTAGTTCGTTTC | | |
| | | | CATGCTGCTGATAAAGACAT | | |
| | | | ATCTGAGACTGGAAACAAAAAGG | | |
| | | | GTTTAATTGGACTTACAGTTCCACA | | |
| | | | TGGCTGGGGAGGCCTCAAAATC | | |
| | | | AGGTGGGAGGCAAAAGGTACTTCT | | |
| | | | TACGTGGTGGCATCAAGAGCAAAA | | |
| | | | TGAGGAAGAAGCAAAAGCAGAA | | |
| | | | ACTCTTCATAAACCCACCAGATCT | | |
| | | | TGTGGGACTTATTATCACGAGAAT | | |
| | | | AGCACAGAAAAGACTGGCCTCC | | |
| | | | ATGATTCAATTACCTCCCACTGCGT | | |
| | | | CCCTCCCACAACATGTGGGAATTC | | |
| | | | TGGGAGATACAATTCAAGTTG | | |
| | | | AGATTTGGGTGGGGACACAGCCAA | | |
| | | | ACCATATCATTCCTCCCTGGGCTCC | | |
| | | | TCCAAATTTCATAATCCTCAC | | |
| | | | ATTTCAAAACCAATCATTCCTTCCC | | |
| | | | AACAGTTCCCCAAAGTCTTAACTC | | |
| | | | ATTTCAGCATTAACCCAAAAG | | |
| | | | TCCACAGTCCAAAGTCTCATCTGA | | |
| | | | GACAAGGCAAGTCCCTTCCACTTA | | |
| | | | CAAGCCTGTAAAAGCAAGCTAG | | |
| | | | TTACCTCCTAGATACAATGGGGGG | | |
| | | | TACAGGTATTGGGTAAATACAGCT | | |
| | | | GTTCCAAATGAGAGAAATTGGC | | |
| | | | CAAAACAAAGGGGTTACAGCGTCC | | |
| | | | ATGCAAGTCTGAAATCCAGTGGGG | | |
| | | | CAGTCAAATTTTAAAGCTCCAT | | |
| | | | AATGATCTCCTTTGACTCCATGTCT | | |
| | | | CACATTCAGGTCATGCTGATGCAA | | |
| | | | GAGATAGGTTCCCATGGTCTT | | |
| | | | GTGCAGCTCCGCCCCTGTGGCTTT | | |
| | | | GCAGAGTACAGCCTCCCTCCTGGC | | |
| | | | TGCTTTCTCAGGCTGATGTTGA | | |
| | | | GTGTCTGTAGCTTTTCCAGGCACA | | |
| | | | AGATGCAAGTTGGTGGTTGATCTA | | |
| | | | CCATTCTGGGGTCTACCATTCT | | |
| | | | GGGGTCTACCGTTCTGGGACTGTG | | |
| | | | GCCTTCTTCTCACAGCTCCACTAGG | | |
| | | | CAGTGCCCCAACAGGGACTCT | | |
| | | | GTGTGGGGGCTCTGCCCCACATTT | | |
| | | | CCCTTCCACACTGCCCTAGGAGAG | | |
| | | | GTTCCCCATGAGGGCTCTGCCC | | |
| | | | CTGCAGCAAACTTTTGCCTGGACA | | |
| | | | TCCAGGTGTTTCCATATATATTCTG | | |
| | | | AAATCTAGGCAGAGGTTCCCA | | |
| | | | AATCTCAATTCTTGACATCTCTGCA | | |
| | | | CCCACAGGCTCAACATCACATGGA | | |
| | | | AGCTGCCAATGCTTGGGGCCT | | |
| | | | CTACCCTCTGAAGCCACAGCCCAA | | |
| | | | GCTCTATGTTGGCTCCTTTCAGCCA | | |
| | | | TGGCTGGAGCAGCTGGGACAC | | |
| | | | AGGGCACCAAGTCCCTAGGCTGCA | | |
| | | | CACAGCACAGAGACCCTGGCCCCA | | |
| | | | GCCCACAAAACCACTTTTTCCT | | |
| | | | CCTGGGCCTCTGGGCCTGTGATGG | | |
| | | | GAGGGGCTGCCATGAAGGTCTCTG | | |
| | | | ACATGACCTGGAGACATTTTCC | | |
| | | | CCATGGTCTTGGGGATTAACATTA | | |
| | | | GGCTCCTTGCTGCTTATGCAAATTT | | |
| | | | CTGCAGCCAGCTTGAATTTCT | | |
| | | | CCTTAAAAAAAATGGGTTTTTCTTT | | |
| | | | TCTACTGCATCATCAGGCTGCAGA | | |
| | | | TTTTCCACATTTATGCTCTTG | | |
| | | | TTTCCCTTTTAAAACAGAATGTTTT | | |
| | | | TAACAGCACCCAAGTCACCTTTTG | | |
| | | | AATGCTTTGCTGCTTAGAAAT | | |
| | | | TTATTCCACCAGATACCCTAAGTC | | |
| | | | ATCTCTCTCAAGCTCTAAGTTCCAC | | |
| | | | AAATCTCTAGGGCAAGGGTGA | | |
| | | | AATGCTGCCAGTCTCCTTGCTAAA | | |
| | | | ACATAACAAGGGTCACCTTTACTT | | |
| | | | CAGTTCCCAACAAGGTCTTCAT | | |
| | | | CTCCATCTGAGACCACCTCAGCCT | | |
| | | | GGACCTTATTGTTCATATCACTATC | | |
| | | | AGTATTTTTGTCAATGCCATT | | |
| | | | CACAGTCTCTAGGAGGTTCCAAAC | | |
| | | | TTTCCTACATTTTCCTATCTTCTTCT | | |
| | | | GAGCCCTCCAGATTATTTCA | | |
| | | | ACACCCAGTTCCAAAGTTGCTTCC | | |
| | | | ACATTTTCGGGTATCTTTTCAGCAA | | |
| | | | TGCCCCACTCTACTGGTACTA | | |
| | | | TTAGTCCATTTTCATGCTGCTGATA | | |
| | | | AAGACATACCTGAGACTGGGAACA | | |
| | | | AAAAGAGGTTTAATTGGACTT | | |
| | | | ATAGTTCCACCTCGCTGGGGAGGC | | |
| | | | CTCAGAATCATGGCAGGAGGTGAA | | |
| | | | AGGCATTTCTTACACGGCAGCA | | |
| | | | GCAAGAGAAAAATGAAGAAGCAG | | |
| | | | CAAAAGCAGAAACCCCTGATAAA | | |
| | | | ACCATCAGATCTCGTGAGACTTAT | | |
| | | | TCACTATCACAAGAATAGCATGGG | | |
| | | | AAAGACCAGCCCCCTTGATTCAAT | | |
| | | | TACCTCCCCCTGGGTCCTGTGG | | |
| | | | GAATTCTGGAAGGTACAATTCAAG | | |
| | | | TTGAGATTTGGGTGGGGACACAGC | | |
| | | | CAAACCATATCAATGATTTTGT | | |
| | | | ACTTTAACCAGCTGAATGGAAGTA | | |
| | | | CAATCTCTTGCTATATCACACAAT | | |
| | | | AATTATTTGCAAAATGAGTAAA | | |
| | | | CATATCATAAGGAAATTATTTTTAC | | |
| | | | AAGGTTTGAAACCTGAAATGCAGT | | |
| | | | CTATTATCATACATAACTAAA | | |
| | | | AATAGAGCCTCAATAAACAGATTC | | |
| | | | CCAGTTTTGAAAATGCAACATTTG | | |
| | | | TACTCCACATTGTCAGTTTTCT | | |
| | | | TAGGTATATTTATAAATACTCCTAT | | |
| | | | AAAAATGTAAAGAAACACATAATG | | |
| | | | TAGATTGCTAATTTTATAATA | | |
| | | | ACACAAGTTGATTTTGACATCCAA | | |
| | | | CTTATTAATTATGAAATGACTTTTG | | |
| | | | GCCTAGTAACAATGAAAATGG | | |
| | | | GGGCAAATACAGATAAATGGTAAT | | |
| | | | TCTTAGAATGAACTACTCAGCACC | | |
| | | | AATTCTAAGTTTTTCTTGATGG | | |
| | | | TAAATCATAATGTTCCCTTTCTCCT | | |
| | | | CGGTTCTGCAATCTATAGGCATAC | | |
| | | | CATAATTGTAATCAATAGCTT | | |
| | | | AAAAATATGTCTCTCTGTCCTATTC | | |
| | | | TGTATCTGTATCTCTTGGATTTTTA | | |
| | | | CCTTTGCAATAGTCAACTGA | | |
| | | | ACCATCTTCTTGGAGTACTCATGA | | |
| | | | AGATGGAAGTCTACATGGAGAATA | | |
| | | | CAGGATGAATCCACTCTGTCTC | | |
| | | | CTGCAGTGAAGTCTGTTTGAAGGA | | |
| | | | TGTATTTGGCTGTCTTCTGGACAGG | | |
| | | | CCATTCTAATAACAGAAACAA | | |
| | | | ACAAGTTATTTTAAAACTTATTGG | | |
| | | | AATATTCAAATATTAACCAAAGTA | | |
| | | | GAAAAATATAATACACATCCAT | | |
| | | | GTGCCCATCACAGAACTTCACTGA | | |
| | | | TTATCATCATTTAGCCAGTCTTGAA | | |
| | | | GAAGCAAGTGCTAATTACAAT | | |
| | | | CACAAATGAAACAAGATTCAGACT | | |
| | | | TCATGAAGAGCACTGCGCTATAAT | | |
| | | | AAAAGAAGAAATGAGCACATAC | | |
| | | | ATTCTTTTACTGACAGTCAAATGGT | | |
| | | | GAAGGTGGCCAGAATCATTATGTG | | |
| | | | ATGCAACATGGCAAAAGTATA | | |
| | | | CAGACAGTGCATCCAGAGGAAGG | | |
| | | | CACCTTGCTGAATGACTAGAATGG | | |
| | | | AAGTAGGAGACATTTTGCAGGCC | | |
| | | | CCCTTCATCCTGCAGGGAGAACCA | | |
| | | | GAACCACAGCAGCTCTATTTGCCT | | |
| | | | ATTCCTCTTTAAATTACAAAGT | | |
| | | | TAAAATTTGGGACTAGTAGAAAAT | | |
| | | | CAATTGGTTATCTTATAGAGTCTCC | | |
| | | | TAGAATATTTCATTGGCATTG | | |
| | | | AGAAGGTGGAAAATGCAAATTATA | | |
| | | | TACTTTAAAATGTAATTTTTGCTTT | | |
| | | | TCACATATGCTTAAAGCCTAA | | |
| | | | AACCTCTTAATAAACTTCTTCTTGAA | | |
| | | | ATATA (SEQ ID NO: 35) | | |
| | | NM_001164178.1 | CCTGTTGCTCTTTGCTCTAATGACC | NP_001157650.1 | MGREELFLTFSFSSGFQ |
| | | | CTTGAGAAAGGATTGCTGGTCATG | | ESNVKTFCSKNILAILGF |
| | | | GGACCAGAGGCTTTATGGGGA | | SSIIAVIAL |
| | | | GGGAAGAACTGTTCTTGACTTTCA | | LAVGLTQNKALPENVK |
| | | | GTTTTTCGAGCGGGTTTCAAGACT | | YGIVLDAGSSHTSLYIY |
| | | | CTAACGTGAAGACATTTTGCTC | | KWPAEKENDTGVVHQV |
| | | | CAAGAATATCCTAGCCATCCTTGG | | EECRVKGPG |
| | | | CTTCTCCTCTATCATAGCTGTGATA | | ISKFVQKVNEIGIYLTDC |
| | | | GCTTTGCTTGCTGTGGGGTTG | | MERAREVIPRSQHQETP |
| | | | ACCCAGAACAAACCATTGCCAGAA | | VYLGATAGMRLLRMES |
| | | | AACGTTAAGTATGGGATTGTGCTG | | EELADRV |
| | | | GATGCGGGTTCTTCTCACACAA | | LDVVERSLSNYPFDFQG |
| | | | GTTTATACATCTATAAGTGGCCAG | | ARIITGQEEGAYGWITIN |
| | | | CAGAAAAGGAGAATGACACAGGC | | YLLGKFSQKTRWFSIVP |
| | | | GTGGTGCATCAAGTAGAAGAATG | | YETNNQ |
| | | | CAGGGTTAAAGGTCCTGGAATCTC | | ETFGALDLGGASTQVTF |
| | | | AAAATTTGTTCAGAAAGTAAATGA | | VPQNQTIESPDNALQFR |
| | | | AATAGGCATTTACCTGACTGAT | | LYGKDYNVYTHSFLCY |
| | | | TGCATGGAAAGAGCTAGGGAAGTG | | GKDQALWQ |
| | | | ATTCCAAGGTCCCAGCACCAAGAG | | KLAKDIQVASNEILRDP |
| | | | ACACCCGTTTACCTGGGAGCCA | | CFHPGYKKVVNVSDLY |
| | | | CGGCAGGCATGCCGTTGCTCAGGA | | KTPCTKRFEMTLPFQQF |
| | | | TGGAAAGTGAAGAGTTGGCAGACA | | EIQGIGNY |
| | | | GGGTTCTGGATGTGGTGGAGAG | | QQCHQSILELFNTSYCP |
| | | | GAGCCTCAGCAACTACCCCTTTGA | | YSQCAFNGIFLPPLQGD |
| | | | CTTCCAGGGTGCCAGGATCATTAC | | FGAFSAFYFVMKFLNLT |
| | | | TGGCCAAGAGGAAGGTGCCTAT | | SEKVSQE |
| | | | GGCTGGATTACTATCAACTATCTG | | KVTEMMKKFCAQPWE |
| | | | CTGGGCAAATTCAGTCAGAAAACA | | EIKTSYAGVKEKYLSEY |
| | | | AGGTGGTTCAGCATAGTCCCAT | | CFSGTYILSLLLQGYHFT |
| | | | ATGAAACCAATAATCAGGAAACCT | | ADSWEHIH |
| | | | TTGGAGCTTTGGACCTTGGGGGAG | | FIGKIQGSDAGWTLGY |
| | | | CCTCTACACAAGTCACTTTTGT | | MLNLTNMIPAEQPLSTP |
| | | | ACCCCAAAACCAGACTATCGAGTC | | LSHSTYVFLMVLFSLVL |
| | | | CCCAGATAATGCTCTGCAATTTCG | | FTVAIIGL |
| | | | CCTCTATGGCAAGGACTACAAT | | LIFHKPSYFWKDMV |
| | | | GTCTACACACATAGCTTCTTGTGCT | | (SEQ ID NO: 38) |
| | | | ATGGGAAGGATCAGGCACTCTGGC | | |
| | | | AGAAACTGGCCAAGGACATTC | | |
| | | | AGGTTGCAAGTAATGAAATTCTCA | | |
| | | | GGGACCCATGCTTTCATCCTGGAT | | |
| | | | ATAAGAAGGTAGTGAACGTAAG | | |
| | | | TGACCTTTACAAGACCCCCTGCAC | | |
| | | | CAAGAGATTTGAGATGACTCTTCC | | |
| | | | ATTCCAGCAGTTTGAAATCCAG | | |
| | | | GGTATTGGAAACTATCAACAATGC | | |
| | | | CATCAAAGCATCCTGGAGCTCTTC | | |
| | | | AACACCAGTTACTGCCCTTACT | | |
| | | | CCCAGTGTGCCTTCAATGGGATTTT | | |
| | | | CTTGCCACCACTCCAGGGGGATTT | | |
| | | | TGGGGCATTTTCAGCTTTTTA | | |
| | | | CTTTGTGATGAAGTTTTTAAACTTG | | |
| | | | ACATCAGAGAAAGTCTCTCAGGAA | | |
| | | | AAGGTGACTGAGATCATGAAA | | |
| | | | AAGTTCTGTGCTCAGCCTTGGGAG | | |
| | | | CAGATAAAAACATCTTACGCTGGA | | |
| | | | GTAAAGGAGAAGTACCTGAGTG | | |
| | | | AATACTGCTTTTCTGGTACCTACAT | | |
| | | | TCTCTCCCTCCTTCTGCAAGGCTAT | | |
| | | | CATTTCACAGCTGATTCCTG | | |
| | | | GGAGCACATCCATTTCATTGGCAA | | |
| | | | GATCCAGGGCAGCGACGCCGGCTG | | |
| | | | GACTTTGGGCTACATGCTGAAC | | |
| | | | CTGACCAACATGATCCCAGCTGAG | | |
| | | | CAACCATTGTCCACACCTCTCTCCC | | |
| | | | ACTCCACCTATGTCTTCCTCA | | |
| | | | TGGTTCTATTCTCCCTGGTCCTTTT | | |
| | | | CACAGTGGCCATCATAGGCTTGCT | | |
| | | | TATCTTTCACAAGCCTTCATA | | |
| | | | TTTCTGGAAAGATATGGTATAGCA | | |
| | | | AAAGCACCTGAAATATGCTGGCTG | | |
| | | | GAGTGAGGAAAAAAATCGTCCA | | |
| | | | GGGAGCATTTTCCTCCATCGCAGT | | |
| | | | GTTCAAGGCCATCCTTCCCTGTCTG | | |
| | | | CCAGGGCCAGTCTTGACGAGT | | |
| | | | GTGAAGCTTCCTTGGCTTTTACTGA | | |
| | | | AGCCTTTCTTTTGGAGGTATTCAAT | | |
| | | | ATCCTTTGCCTCAAGGACTT | | |
| | | | CGGCAGATACTGTCTCTTTCATGA | | |
| | | | GTTTTTCCCAGCTACACCTTTCTCC | | |
| | | | TTTGTACTTTGTGCTTGTATA | | |
| | | | GGTTTTAAAGACCTGACACCTTTC | | |
| | | | ATAATCTTTGCTTTATAAAAGAAC | | |
| | | | AATATTGACTTTGTCTAGAAGA | | |
| | | | ACTGAGAGTCTTCAGTCCTGTGAT | | |
| | | | AGGAGGCTGAGCTGGCTGAAAGA | | |
| | | | AGAATCTCAGGAACTGGTTCAGT | | |
| | | | TGTACTCTTTAAGAACCCCTTTCTC | | |
| | | | TCTCCTGTTTGCCATCCATTAAGAA | | |
| | | | AGCCATATGATGCCTTTGGA | | |
| | | | GAAGGCAGACACACATTCCATTCC | | |
| | | | CAGCCTGCTCTGTGGGTAGGAGAA | | |
| | | | TTTTCTACAGTAGGCAAATATG | | |
| | | | TGCTAAAGCCAAAGAGTTTTATAA | | |
| | | | GGAAATATATGTGCTCATGCAGTC | | |
| | | | AATACAGTTCTCAATCCCACCC | | |
| | | | AAAGCAGGTATGTCAATAAATCAC | | |
| | | | ATATTCCTAGGTGATACCCAAATG | | |
| | | | CTACAGAGTGGAACACTCAGAC | | |
| | | | CTGAGATTTGCAAAAAGCAGATGT | | |
| | | | AAATATATGCATTCAAACATCAGG | | |
| | | | GCTTACTATGAGGTAGGTGGTA | | |
| | | | TATACATGTCACAAATAAAAATAC | | |
| | | | AGTTACAACTCAGGGTCACAAAAA | | |
| | | | ATGCATCTTCCAATGCATATTT | | |
| | | | TTATTATGGTAAAATATACATAAA | | |
| | | | TATAATTCACCATTTTAACATTTAA | | |
| | | | TTCATATTAAATACGTACAAA | | |
| | | | TCAGTGACATTTAGTACATTCACA | | |
| | | | GTGTTGTGCCACCATCACCACTATT | | |
| | | | TAGTTCCAGAACATTTGCATC | | |
| | | | ATCAATACATTGTCTAGAGACAAG | | |
| | | | ACTATCCTGGGTAGGCAGAAACCA | | |
| | | | TAGATCTTTTGTGTTTACAGCT | | |
| | | | ATGGAAACCAACTGTACCATAAAG | | |
| | | | ATAGTTCACTGAGTTTTAAACCCA | | |
| | | | AGCCACATCTTATTTTTCCAAG | | |
| | | | GTTTAATTTAGTGAGAGGGCAGCA | | |
| | | | TTAGTGTGGAGTGGCATGCTTTTGC | | |
| | | | CCTATCGTGGAATTTACACAT | | |
| | | | CAGAATGTGCAGGATCCAAGTCTG | | |
| | | | AAAGTGTTGCCACCCGTCACACAA | | |
| | | | CATGGGCTTTGTTTGCTTATTC | | |
| | | | CATGAAGCAGCAGCTATAGACCTT | | |
| | | | ACCATGGAAACATGAAGAGACCCT | | |
| | | | CCACCCCTTTCCTTAAGGATTG | | |
| | | | CTGCAAGAGTTACCTGTTGAGCAG | | |
| | | | GATTGACTGGTGATGTTTCATTCTG | | |
| | | | ACCTTGTCCCAAGCTCTCCAT | | |
| | | | CTCTAGATCTGGGGACTGACTGTT | | |
| | | | GAGCTGATGGGGAAAGAAAAGCT | | |
| | | | CTCACACAAACCGGAAGCCAAAT | | |
| | | | GTCCCCTATCTCTTGAATGATCAAG | | |
| | | | TCACTTTTGACAACATCCAGGTGA | | |
| | | | ATATAAAAACTTAATAAAGCT | | |
| | | | GTGGAAAGGAACTCTTAATCTTCT | | |
| | | | TTTCTGCTACTTAGGTTAAATTCAC | | |
| | | | TAGATCTTGATTAGGAATCAA | | |
| | | | AATTCGAATTGGGACATGTTCAAA | | |
| | | | TTCTTTCTTGTGGTAGTTGCCTATA | | |
| | | | CTGTCATCGCTGCTGTTGGTT | | |
| | | | CAGCATTTGTGGTGTACCACGCTG | | |
| | | | TGTGCTCAAGGGTATTACATTCATC | | |
| | | | TTCTCATTTAATCCTCACAAC | | |
| | | | AATCTGAAGAAGGTAGGTATTACA | | |
| | | | ATTCCCACTTCATAGAAACAGAAA | | |
| | | | CTGAGGTTCAGAGAGGTTAAGT | | |
| | | | CATTTGCCCAAATGGCTGAGCCAA | | |
| | | | AGCCTACCATGTACCTAACCTTTAT | | |
| | | | TTTCTTTCCCGAACATACCAG | | |
| | | | GCTGTCTCCTCATAACTTCCAAGC | | |
| | | | ATGCACTTAAAACTCCACATGAAT | | |
| | | | ACAAGGTTCATGGGACTTGGTA | | |
| | | | TTCATAGAAAGGGAGGCAGAAAG | | |
| | | | CTGGTCTGTTCCTGATAGGCTTGTA | | |
| | | | ATTTAATATCATTCTGTTCATG | | |
| | | | TGCTTTGGATGGAAGCACATCTGG | | |
| | | | CATATGATGCTAATCAGTGGTTCC | | |
| | | | CATACCCCTGGCTTCCTAATTT | | |
| | | | TAATGTTTGCTCACAGCATAGTAG | | |
| | | | ATTGACATCAAATAGTGGCCGATG | | |
| | | | ATGATGAAAATAAAGGTCAAAT | | |
| | | | AAGTTGAGCCAATAACAGCCGCTT | | |
| | | | TTTTCCTTCTGTCTGCGTATACAAA | | |
| | | | GCACTGTCATGCACACAATCT | | |
| | | | ATTCTGACCCTCACAACAACCCAT | | |
| | | | AAGGGTGTAAATAGTATTTCCATT | | |
| | | | TTACAAATGAGGATCACACAAA | | |
| | | | CTACTACATGGCAGAGCAGATACT | | |
| | | | CCAACTCATGTCTTCTGGTTGAAGC | | |
| | | | CTATTGCTTTTTCTTTTCTAA | | |
| | | | ACACTTTCCCTCAGCAAGTTGGAA | | |
| | | | TTAGACTTCACAAGTCTCCTTCAGA | | |
| | | | GAACACAAATCTTTTCTTATT | | |
| | | | CCATTCCTGTTTGGTTGCCTACGTC | | |
| | | | CAATCTCCCCCTCCCCAGAGATGC | | |
| | | | CAAAAAAAAAATCCTTTAAGG | | |
| | | | TATTTGGGAGCCAAACTCAACTTG | | |
| | | | TTAAAATCTCAAATTATGGAGACA | | |
| | | | ATCAGCAGACACAACCTAACCC | | |
| | | | CAATTATTTTGGCAGGAAGGTTGG | | |
| | | | TTTAGAGGCAGATCCAGCAATCTG | | |
| | | | CTTTGGGCCACTCTGGGTGGGG | | |
| | | | TAGGTGAAATAAGATTGGTCACTG | | |
| | | | TTAACTAATTTTAATATTGGATTGG | | |
| | | | CCATTGGTTATCACTGATTAC | | |
| | | | CATTCTCCCCTGGATTTTCACCCAG | | |
| | | | GACTCAAAACTTGGTTCTGCTAAC | | |
| | | | CCTGTTCCTTTATGAGGAACC | | |
| | | | TTTTAAAGATTCCTTTATAAGGTGG | | |
| | | | GAGTTTTTTTTCTATGAACCTATAG | | |
| | | | GGGAGAAAAAAGATCAGCAG | | |
| | | | AAGTCATTACTTTTTTTTTTTTTTTT | | |
| | | | TTTTTTTTTTGAGAGAGAGTCTCAC | | |
| | | | TCCATTGCCCAGGCTGGAG | | |
| | | | TGCAGTGGTGCTATCTCGGCTCACT | | |
| | | | GCAACCTCCGCCTCCTGGGTTCAA | | |
| | | | GCAATTCTCCTGCCTCAGCCT | | |
| | | | CCCGAGTAGCTGGGATTGCAGGTG | | |
| | | | CCCACCACCACACCCGGCTAATTT | | |
| | | | TTGTATTTTTAGTAAAGACAGG | | |
| | | | GTTTCACCATGTTGGCCAGGCTGG | | |
| | | | TCTCCAACTCCCAATCTCAGGTGA | | |
| | | | TCCTATTGCCTCGGCCTCCCAA | | |
| | | | AGTGCTGGGATTACAGGAGTGAGC | | |
| | | | CACCATGCCTGGCCAGAAGTGGTT | | |
| | | | ACTTCTGTAGACAAAAGAATAA | | |
| | | | TGCTACTTAATCAGGCTTTCTGTGT | | |
| | | | GACAAGAAAGAGAAAGAAAATAA | | |
| | | | AGAAGTTTCAATTCATCCAATT | | |
| | | | CTTAATAAGAAATATGTAAATAAA | | |
| | | | ATTTTTTAAAATTACACTTCATTTT | | |
| | | | AATGTTGTATCAGTCAAGGTC | | |
| | | | CCTGCAAGAGATGGATGGTATGGT | | |
| | | | ACACTCAAACTGGGTAACACAGGA | | |
| | | | GAGTTTTCAGAAAGCAACTAAA | | |
| | | | TCCAAAATACTATCAAGGAATCAA | | |
| | | | TATAAAAATTGTTAATATTTTTCTC | | |
| | | | ATACTAAATTTTCAAAATATT | | |
| | | | TTGTGTCTATTACATTTACAGCACA | | |
| | | | TCTTAATTAGGACTAGCTGTGTGTT | | |
| | | | CACCTCACATCTGGCTTGTA | | |
| | | | GCTACCATACTGGACAGCACATGT | | |
| | | | CCAAAAAAATACACGTAAAGTTAA | | |
| | | | AGTTTAAAAGACACAGGAACTA | | |
| | | | AGCCCTCATTGTCTTTCCCTTGGGA | | |
| | | | GGTAGTTTAAAGAGCTATAGATGC | | |
| | | | TGTAACATTCTTCCTATTATT | | |
| | | | TATTATATATGACATTATTCCTAAA | | |
| | | | AAAGCTTTTGAGATCCTAGGTTGT | | |
| | | | ATTCCTCAGGTTTTGTTCCCT | | |
| | | | TCCCATGAAGATGTGAAGGCAGGG | | |
| | | | ATGCCTGTTATTCAGTCCAAGATG | | |
| | | | CATGACAAGAGACCTTGGGAAA | | |
| | | | GTTTCATCTGGATTTAAAGATTAAT | | |
| | | | TCTTGATGCTTACATTCCATACTCA | | |
| | | | AAATGTAAATTTGAATATTA | | |
| | | | AAATAAAGATGATTTTTTTTTTGGA | | |
| | | | GCTAGTCTTGCTCTGTTGCCCAGGC | | |
| | | | TGGAATGCAGTGGCATCATC | | |
| | | | ATGGCTCACTGCAGCCTCCACCTC | | |
| | | | CCAAGCTCAAGCAAGGCTACAGGT | | |
| | | | GTGCACCTAAGTAGCTAGGACT | | |
| | | | ACAGGTGTGCACCACCATGTCTAG | | |
| | | | CTATTTTTTTTTCTGTAGACACAGG | | |
| | | | CTTTTCCTATGTTGTCCAGGC | | |
| | | | TGGTCTCGAACTCCTGCCCTCAAG | | |
| | | | CAATCCTCCTGCCTTGGCCTCCCAA | | |
| | | | AGTGTTGAGATTACAGGCGTA | | |
| | | | AGCCACTGCACCTGGCCAAGATGA | | |
| | | | ATATTTTAATACCTCACAGAACAA | | |
| | | | AGTTTGCCACATAATGATAAAA | | |
| | | | TTACTATGAAAATATATTCCCTTTA | | |
| | | | TTGTCAGTTTAAAACATGAACTGA | | |
| | | | GTTTCACCCAAACTGGTCTGG | | |
| | | | CCCCTCTCTGATTCAAATACCAAT | | |
| | | | AGTTGCTCTGATTCAAATTCCAACT | | |
| | | | GTTAGAACATGACAGCTGCTC | | |
| | | | ATAACTAGCTTTGCTTACTAACCAT | | |
| | | | GTTTCTTTCCATTTGTATTAGGTCC | | |
| | | | TTTACTTTTTATAACAGCCT | | |
| | | | CAAAGTTTCATGAATTGCTGCAGT | | |
| | | | AAACATTGATTTTCATGTTTGTGAG | | |
| | | | TCTGCAAGCCAGCTGGGCAGC | | |
| | | | TCTACTTCAGGTGGTAAGGGTGCA | | |
| | | | TCAGACCTATTCCATATACCTCTTG | | |
| | | | TTCTCCTTGTCCAGTGGTTTC | | |
| | | | TAGGGATATGTTCTCATGATGAAC | | |
| | | | CCCGCAGAGGCTCGTGAAAGTGAG | | |
| | | | AGGAAACTAGGATGCCTCTTAA | | |
| | | | GGTCTTGGTCAGGATGGGGTCTCC | | |
| | | | TGTCACTTCTGTCACAGGCTATTGT | | |
| | | | AAGTCATATGAGCAAGCTCAA | | |
| | | | TAAAATATAAACAACTCAGATAAA | | |
| | | | CAGTGGGAGGAATGGCAAAGTCAT | | |
| | | | ATGGCCAAGGCCATGAGTGATT | | |
| | | | AATTTTAACACAGGAAAAAAGTAA | | |
| | | | AGCATTAAATGCGATTATTTAATA | | |
| | | | TACAATGTCTTATTAACTGAAA | | |
| | | | TATAAAATGTGTTTACTGTAAAAT | | |
| | | | ATAATCTGTTTATCTCACCAAAGA | | |
| | | | AATATTATCTTTAAAAAATGTC | | |
| | | | ATTACTTCTAAGACATCATCAGTCT | | |
| | | | GCAACTTCTTTCCATAGCCTTAATC | | |
| | | | AGGATGCTGTGGCAGCTCCC | | |
| | | | ACATTAGCCTCGCATTCTAAACTG | | |
| | | | GTAGATGTCCTAGGAAACCATACA | | |
| | | | TCTATGTATTTTTCTTATTTTA | | |
| | | | TACGTTTAGGACAATGTATACCTA | | |
| | | | ATTACCCAACTTTTTATTTGCATAC | | |
| | | | AAATCTAATACAACTGAACAC | | |
| | | | AATCAGTTTTATCACAGGTATAAT | | |
| | | | GGATTTTTCAATAGTGAGGAGGTG | | |
| | | | CCTCCATGAGCCTTCTCTTTAG | | |
| | | | AAAAGTGGCATTCAAGACTCTTCA | | |
| | | | TTTGAAGTGAAGATTGCTATGTCTT | | |
| | | | TTGCATTGCTCTATTTTACAT | | |
| | | | AAATTAAGTTATAAATTGACACTA | | |
| | | | TAATCAACTGACACCATGATCAGT | | |
| | | | GATGATGATCACCCTCATCACC | | |
| | | | ACTAGAGTTGACTTGTTTTTATAAC | | |
| | | | CCCTTTGCATGTATGTTGAATAGCA | | |
| | | | AAGTTCATCAGAGAACATGT | | |
| | | | ATTAGTCAATGGTAAGTAAGATAC | | |
| | | | TCTCATCTAAGAAATAACATCACC | | |
| | | | TCTTCTAATGAAGTTCTAAGAA | | |
| | | | GAGAGGGAAGAAAAAGTCTTGGG | | |
| | | | AGCTAGTCAGGGAATAGTGTGTAT | | |
| | | | TTGCAATTACCTAAACTGAACTC | | |
| | | | TACCATTACTCCTAACCCAGTTCCT | | |
| | | | CCTCCTGTGTTTTACATGATTAATG | | |
| | | | CCACCCCTGCCTCAATGAAC | | |
| | | | CAAGATCAGCTCCATCACTGGGAC | | |
| | | | CTCCCCATTCTGCCTGTGCAATATT | | |
| | | | TTTCTTTTTTATTTCTCCTTC | | |
| | | | TAATATTACTGTTATTGCTCCAGTA | | |
| | | | AAGAGCTGTAATATATTTTACCTG | | |
| | | | GACTGATACCAGGAATGGTGG | | |
| | | | TGTTGCTTCCAATCTGTTGCTGCTA | | |
| | | | GATTAATCTTTGCAAAGCACAGGC | | |
| | | | TTAATTTCATTGCTGCTCAAC | | |
| | | | TAAAACCACTGGTGGCTTTCCATT | | |
| | | | GCCTACAAAATAAAGTCAACCTCC | | |
| | | | CCATCAGACATTCAAGGCTTTC | | |
| | | | AATGATCCATGGCCGCCAGCTCTC | | |
| | | | TCCAGGCTCATATCCCACTCCACTC | | |
| | | | CTCTGATGTTTCCTACACTAC | | |
| | | | ACTACACTATACTACACTACAGCC | | |
| | | | AGGTAGAATGACTGTTCACCCAAC | | |
| | | | ACCACTCAGGTTGTCTTCTCAA | | |
| | | | CTTGGAATACTCTTGCACCTTCAAA | | |
| | | | GCTCATTTCAAATGCCCCTTCATTT | | |
| | | | GTGAAGCCTTCTCCAAATTT | | |
| | | | CCAAGTCAGAATGTCTCTTCCTTGT | | |
| | | | GCTACCACAACCCTTTAACTGAGC | | |
| | | | CTCCATTAGTGCACTGAGACC | | |
| | | | ATTCTGTTCAGTGTCTGGGTGAAG | | |
| | | | CTTCCTCGTGAAAAATATGTTACCT | | |
| | | | ATTTCTTTCTGAAAAGTTGGA | | |
| | | | TTCAGGGATATTATCACGGACCTA | | |
| | | | AGGTAATAGTTCTAGCCAACCTCC | | |
| | | | CTGTCCACTGCCAGGCCGACTA | | |
| | | | CAAACCCTTCTGTTGCTGGCGAGC | | |
| | | | TGGTCCGCACCACTAGTTCTGCTTC | | |
| | | | ACTCTATTTATCTCTTGATGT | | |
| | | | AACCATCTTCTTTCTCCAGGTTTTA | | |
| | | | AGAACCAGCCCAACTCCTGGTTCC | | |
| | | | CTGATGAAGCTTTTATTCCCC | | |
| | | | TAGCCACATGGAACTTTTCCTTTTT | | |
| | | | GGAACATGCCTTTAGTTTCTGTGTA | | |
| | | | GTTTGCCATGCAGCACTTCA | | |
| | | | TTGTACACATTATTAAAACAGAAT | | |
| | | | TTTAAGGATTAGAATGAACCTTAA | | |
| | | | AAGATCATGCATCTCAAAATTT | | |
| | | | AATGTACATACAAATTACCCAGGG | | |
| | | | ATTTTGTTGAAATAAAAATTATTTA | | |
| | | | ATTTTAATTAATATAAATAAT | | |
| | | | TCAGTAGGTCTGGGGTGAGGCCTG | | |
| | | | AGGTTTTACATTTCCAACAAGCTG | | |
| | | | CCAGGTAAAGCCAATACATCTG | | |
| | | | TCCAGGAATCACACTTTGCGTATC | | |
| | | | AAAGGTCTAGATGACATTATCATT | | |
| | | | CCAAAGAGTTTCTTTTACAGGC | | |
| | | | TCTCAGATCAGTGTTCATCCACTAC | | |
| | | | CTGACTACTGTCATTCACAGGCATT | | |
| | | | CTGTTCCACAGCAGGCCAGC | | |
| | | | TAACGTGGTATTTACAAAGCTCAC | | |
| | | | TCCTCTTATACAACAATCCAAGTGT | | |
| | | | TTCTTTTGTCAGTTGTCTGTG | | |
| | | | CCCCAGGAGATCCCTCTCTGCCTT | | |
| | | | GCCTTGCCCTCTGCCTTTGGAGACC | | |
| | | | AGCACCTCATACTCAGTGAAG | | |
| | | | GCCTGGAGTGCTTAAGAGGGATTT | | |
| | | | CTTCCAGCTCTCTTGCCCTGGTCTT | | |
| | | | CAGTGTATTAGATGTATTACC | | |
| | | | TCCATGCTCTCAGTAGAGGCCCAT | | |
| | | | AGGAAAGAGTAGGTAGGTTATGCC | | |
| | | | AGCTCACACGCATCCTTTAAAA | | |
| | | | ATGGTTTAGAAGTTTAGCTGGTTTC | | |
| | | | TTATTACTCCTGTCTATGGATGTTT | | |
| | | | CCTTCTGTCACTCTACTAGG | | |
| | | | CATGAAACAGCTAATCATGTTCAA | | |
| | | | TAGTTACATTTAGATTGGTTTTTAA | | |
| | | | AAACTATGATTGTATTAGTTC | | |
| | | | GTTTCCATGCTGCTGATAAAGACA | | |
| | | | TATCTGAGACTGGAAACAAAAAGG | | |
| | | | GTTTAATTGGACTTACAGTTCC | | |
| | | | ACATGGCTGGGGAGGCCTCAAAAT | | |
| | | | CAGGTGGGAGGCAAAAGGTACTTC | | |
| | | | TTACGTGGTGGCATCAAGAGCA | | |
| | | | AAATGAGGAAGAAGCAAAAGCAG | | |
| | | | AAACTCTTCATAAACCCACCAGAT | | |
| | | | CTTGTGGGACTTATTATCACGAG | | |
| | | | AATAGCACAGAAAAGACTGGCCTC | | |
| | | | CATGATTCAATTACCTCCCACTGC | | |
| | | | GTCCCTCCCACAACATGTGGGA | | |
| | | | ATTCTGGGAGATACAATTCAAGTT | | |
| | | | GAGATTTGGGTGGGGACACACCCA | | |
| | | | AACCATATCATTCCTCCCTGGG | | |
| | | | CTCCTCCAAATTTCATAATCCTCAC | | |
| | | | ATTTCAAAACCAATCATTCCTTCCC | | |
| | | | AACAGTTCCCCAAAGTCTTA | | |
| | | | ACTCATTTCAGCATTAACCCAAAA | | |
| | | | GTCCACAGTCCAAAGTCTCATCTG | | |
| | | | AGACAAGGCAAGTCCCTTCCAC | | |
| | | | TTACAAGCCTGTAAAAGCAAGCTA | | |
| | | | GTTACCTCCTAGATACAATGGGGG | | |
| | | | GTACAGGTATTGGGTAAATACA | | |
| | | | GCTGTTCCAAATGAGAGAAATTGG | | |
| | | | CCAAAACAAAGGGGTTACAGGGTC | | |
| | | | CATGCAAGTCTGAAATCCAGTG | | |
| | | | GGGCAGTCAAATTTTAAAGCTCCA | | |
| | | | TAATGATCTCCTTTGACTCCATGTC | | |
| | | | TCACATTCAGGTCATGCTGAT | | |
| | | | GCAAGAGATAGGTTCCCATGGTCT | | |
| | | | TGTGCAGCTCCGCCCCTGTGGCTTT | | |
| | | | GCAGAGTACAGCCTCCCTCCT | | |
| | | | GGCTGCTTTCTCAGGCTGATGTTGA | | |
| | | | GTGTCTGTAGCTTTTCCAGGCACA | | |
| | | | AGATGCAAGTTGGTGGTTGAT | | |
| | | | CTACCATTCTGGGGTCTACCATTCT | | |
| | | | GGGGTCTACCGTTCTGGGACTGTG | | |
| | | | GCCTTCTTCTCACAGCTCCAC | | |
| | | | TAGGCAGTGCCCCAACAGGGACTC | | |
| | | | TGTGTGGGGGCTCTGCCCCACATTT | | |
| | | | CCCTTCCACACTGCCCTAGGA | | |
| | | | GAGGTTCCCCATGAGGGCTCTGCC | | |
| | | | CCTGCAGCAAACTTTTGCCTGGAC | | |
| | | | ATCCAGGTGTTTCCATATATAT | | |
| | | | TCTGAAATCTAGGCAGAGGTTCCC | | |
| | | | AAATCTCAATTCTTGACATCTCTGC | | |
| | | | ACCCACAGGCTCAACATCACA | | |
| | | | TGGAAGCTGCCAATGCTTGGGGCC | | |
| | | | TCTACCCTCTGAAGCCACAGCCCA | | |
| | | | AGCTCTATGTTGGCTCCTTTCA | | |
| | | | GCCATGGCTGGAGCAGCTGGGACA | | |
| | | | CAGGGCACCAAGTCCCTAGGCTGC | | |
| | | | ACACAGCACAGAGACCCTGGGC | | |
| | | | CCAGCCCACAAAACCACTTTTTCC | | |
| | | | TCCTGGGCCTCTGGGCCTGTGATG | | |
| | | | GGAGGGGCTGCCATGAAGGTCT | | |
| | | | CTGACATGACCTGGAGACATTTTC | | |
| | | | CCCATGGTCTTGGGGATTAACATT | | |
| | | | AGGCTCCTTGCTCCTTATGCAA | | |
| | | | ATTTCTGCAGCCAGCTTGAATTTCT | | |
| | | | CCTTAAAAAAAATGGGTTTTTCTTT | | |
| | | | TCTACTGCATCATCAGGCTG | | |
| | | | CAGATTTTCCACATTTATGCTCTTG | | |
| | | | TTTCCCTTTTAAAACAGAATCTTTT | | |
| | | | TAACAGCACCCAAGTCACCT | | |
| | | | TTTGAATGCTTTGCTGCTTAGAAAT | | |
| | | | TTATTCCACCAGATACCCTAAGTC | | |
| | | | ATCTCTCTCAAGCTCTAAGTT | | |
| | | | CCACAAATCTCTAGGGCAAGGGTG | | |
| | | | AAATGCTGCCAGTCTCCTTGCTAA | | |
| | | | AACATAACAAGGGTCACCTTTA | | |
| | | | CTTCAGTTCCCAACAAGGTCTTCAT | | |
| | | | CTCCATCTGAGACCACCTCAGCCT | | |
| | | | GGACCTTATTGTTCATATCAC | | |
| | | | TATCAGTATTTTTGTCAATGCCATT | | |
| | | | CACAGTCTCTAGGAGGTTCCAAAC | | |
| | | | TTTCCTACATTTTCCTATCTT | | |
| | | | CTTCTGAGCCCTCCAGATTATTTCA | | |
| | | | ACACCCAGTTCCAAAGTTGCTTCC | | |
| | | | ACATTTTCGGGTATCTTTTCA | | |
| | | | GCAATGCCCCACTCTACTGGTACT | | |
| | | | ATTACTCCATTTTCATGCTGCTGAT | | |
| | | | AAAGACATACCTGAGACTGGG | | |
| | | | AACAAAAAGAGGTTTAATTGGACT | | |
| | | | TATAGTTCCACCTGGCTGGGGAGG | | |
| | | | CCTCAGAATCATGGCAGGAGGT | | |
| | | | GAAAGGCATTTCTTACACGGCAGC | | |
| | | | AGCAAGAGAAAAATGAAGAAGCA | | |
| | | | GCAAAAGCAGAAACCCCTGATAA | | |
| | | | AACCATCAGATCTCGTGAGACTTA | | |
| | | | TTCACTATCACAAGAATAGCATGG | | |
| | | | GAAAGACCAGCCCCCTTGATTC | | |
| | | | AATTACCTCCCCCTGGGTCCTGTG | | |
| | | | GGAATTCTGGAAGGTACAATTCAA | | |
| | | | GTTGAGATTTGGGTGGGGACAC | | |
| | | | AGCCAAACCATATCAATGATTTTG | | |
| | | | TACTTTAACCAGCTGAATGGAAGT | | |
| | | | ACAATCTCTTGCTATATGACAC | | |
| | | | AATAATTATTTGCAAAATGAGTAA | | |
| | | | ACATATCATAAGCAAATTATTTTT | | |
| | | | ACAAGGTTTGAAACCTGAAATG | | |
| | | | CAGTCTATTATCATACATAACTAA | | |
| | | | AAATAGAGCCTCAATAAACAGATT | | |
| | | | CCCAGTTTTGAAAATGCAACAT | | |
| | | | TTGTACTCCACATTGTCAGTTTTCT | | |
| | | | TAGGTATATTTATAAATACTCCTAT | | |
| | | | AAAAATGTAAAGAAACACAT | | |
| | | | AATGTAGATTGCTAATTTTATAATA | | |
| | | | ACACAAGTTGATTTTGACATCCAA | | |
| | | | CTTATTAATTATGAAATGACT | | |
| | | | TTTGGCCTAGTAACAATGAAAATG | | |
| | | | GGGGCAAATACAGATAAATGGTAA | | |
| | | | TTCTTAGAATGAACTACTCAGC | | |
| | | | ACCAATTCTAAGTTTTTCTTGATGG | | |
| | | | TAAATCATAATGTTCCCTTTCTCCT | | |
| | | | CGGTTCTGCAATCTATAGGC | | |
| | | | ATACCATAATTGTAATCAATAGCT | | |
| | | | TAAAAATATGTCTCTCTGTCCTATT | | |
| | | | CTGTATCTCTATCTCTTGGAT | | |
| | | | TTTTACCTTTGCAATAGTCAACTGA | | |
| | | | ACCATCTTCTTGGAGTACTCATGA | | |
| | | | AGATGGAAGTCTACATGGAGA | | |
| | | | ATACAGGATGAATCCACTCTGTCT | | |
| | | | CCTGCAGTGAAGTCTGTTTGAAGG | | |
| | | | ATGTATTGGCTGTCTTCTGGA | | |
| | | | CAGGCCATTCTAATAACAGAAACA | | |
| | | | AACAAGTTATTTTAAAACTTATTG | | |
| | | | GAATATTCAAATATTAACCAAA | | |
| | | | GTAGAAAAATATAATACACATCCA | | |
| | | | TGTGCCCATCACAGAACTTCACTG | | |
| | | | ATTATCATCATTTAGCCAGTCT | | |
| | | | TGAAGAAGCAAGTGCTAATTACAA | | |
| | | | TCACAAATGAAACAAGATTCAGAC | | |
| | | | TTCATGAAGAGCACTGCGCTAT | | |
| | | | AATAAAAGAAGAAATGAGCACAT | | |
| | | | ACATTCTTTTACTGACAGTCAAATG | | |
| | | | GTGAAGGTGGGCAGAATCATTA | | |
| | | | TGTGATGCAACATGGCAAAAGTAT | | |
| | | | ACAGACAGTGCATCCAGAGGAAG | | |
| | | | GCACCTTGCTGAATGACTAGAAT | | |
| | | | GGAAGTAGGAGACATTTTGCAGGC | | |
| | | | CCCCTTCATCCTGCAGGGAGAACC | | |
| | | | AGAACCACAGCAGCTCTATTTG | | |
| | | | CCTATTCCTCTTTAAATTACAAAGT | | |
| | | | TAAAATTTGGGAGTAGTAGAAAAT | | |
| | | | CAATTGGTTATCTTATAGAGT | | |
| | | | CTCCTAGAATATTTCATTGGCATTG | | |
| | | | AGAAGGTGGAAAATGCAAATTATA | | |
| | | | TACTTTAAAATGTAATTTTTG | | |
| | | | CTTTTCACATATGCTTAAAGCCTAA | | |
| | | | AACCTCTTAATAAACTTCTTCTGAA | | |
| | | | ATATA (SEQ ID NO: 37) | | |
| | | NM_001164179.2 | ACGGAGACGGACCACAGCAAGCA | NP_001157651.1 | MEDTKESNVKTFCSKNI |
| | | | GAGGCTGGGGGGGGGAAAGACGA | | LAILGESSHAVIALLAVG |
| | | | GGAAAGAGGAGGAAAACAAAAGC | | LTQNKALP |
| | | | T | | ENVKYGIVLDAGSSHTS |
| | | | GCTACTTATGGAAGATACAAAGCA | | LYTYKWPAEKENDTGV |
| | | | GTCTAACGTGAAGACATTTTGCTC | | VHQVEECRVKGPGISKF |
| | | | CAAGAATATCCTAGCCATCCTT | | VQKVNEIG |
| | | | GGCTTCTCCTCTATCATAGCTGTGA | | IYLTDCMERAREVIPRS |
| | | | TAGCTTTGCTTGCTGTGGGGTTGAC | | QHQETPVYLGATAGMR |
| | | | CCAGAACAAAGCATTGCCAG | | LLRMESEELADRVLDV |
| | | | AAAACGTTAAGTATGGGATTGTGC | | VERSLSNYP |
| | | | TGGATGCGGGTTCTTCTCACACAA | | FDFQGARIITGQEEGAY |
| | | | GTTTATACATCTATAAGTGGCC | | GWITINYLLGKFSQKIR |
| | | | AGCAGAAAAGGAGAATGACACAG | | WFSIVPYETNNQETFGA |
| | | | GCGTGGTGCATCAAGTAGAAGAAT | | LDLGGAS |
| | | | GCAGGGTTAAAGGTCCTGGAATC | | TQVTFVPQNQTIESPDN |
| | | | TCAAAATTTGTTCAGAAAGTAAAT | | ALQFRLYGKDYNVYTH |
| | | | GAAATAGGCATTTACCTGACTGAT | | SFLCYGKDQALWQKLA |
| | | | TGCATGGAAAGAGCTAGGGAAG | | KDIQQFEIQ |
| | | | TGATTCCAAGGTCCCAGCACCAAG | | GIGNYQQCHQSILELFN |
| | | | AGACACCCGTTTACCTGGGAGCCA | | TSYCPYSQCAFNGIFLPP |
| | | | CGGCAGGCATGCGGTTGCTCAG | | LQGDFGAFSAFYFVMK |
| | | | GATGGAAAGTGAAGAGTTGGCAG | | FLNLTSE |
| | | | ACAGGGTTCTGGATGTGGTGGAGA | | KVSQEKVTEMMKKFCA |
| | | | GGAGCCTCAGCAACTACCCCTTT | | QPWEEIKTSYAGVKEK |
| | | | GACTTCCAGGGTGCCAGGATCATT | | YLSEYCFSGTYILSLLLQ |
| | | | ACTGGCCAAGAGGAAGGTGCCTAT | | GYHFTADS |
| | | | GGCTGGATTACTATCAACTATC | | WEHIHFIGKIQGSDAGW |
| | | | TGCTGGCCAAATTCACTCAGAAAA | | TLGYMLNLTNMIPAEQP |
| | | | CAAGGTGGTTCAGCATAGTCCCAT | | LSTPLSHSTYVFLMVLF |
| | | | ATGAAACCAATAATCAGGAAAC | | SLVLFTV |
| | | | CTTTGGAGCTTTGGACCTTGGGGG | | AIIGLLIFHKPSYFWKD |
| | | | AGCCTCTACACAAGTCACTTTTGTA | | MV (SEQ ID NO: 40) |
| | | | CCCCAAAACCAGACTATCGAG | | |
| | | | TCCCCAGATAATGCTCTGCAATTTC | | |
| | | | GCCTCTATGGCAAGGACTACAATG | | |
| | | | TCTACACACATAGCTTCTTGT | | |
| | | | GCTATGGGAAGGATCAGGCACTCT | | |
| | | | GGCAGAAACTGGCCAAGGACATTC | | |
| | | | AGCAGTTTGAAATCCAGGGTAT | | |
| | | | TGGAAACTATCAACAATGCCATCA | | |
| | | | AAGCATCCTGGAGCTCTTCAACAC | | |
| | | | CAGTTACTGCCCTTACTCCCAG | | |
| | | | TGTGCCTTCAATGGGATTTTCTTGC | | |
| | | | CACCACTCCAGGGGGATTTTGGGG | | |
| | | | CATTTTCAGCTTTTTACTTTG | | |
| | | | TGATGAAGTTTTTAAACTTGACATC | | |
| | | | AGAGAAAGTCTCTCAGGAAAAGGT | | |
| | | | GACTGAGATGATGAAAAAGTT | | |
| | | | CTGTGCTCAGCCTTGGGAGGAGAT | | |
| | | | AAAAACATCTTACGCTGGAGTAAA | | |
| | | | GGAGAAGTACCTGAGTGAATAC | | |
| | | | TGCTTTTCTGGTACCTACATTCTCT | | |
| | | | CCCTCCTTCTGCAAGGCTATCATTT | | |
| | | | CACAGCTGATTCCTGGGAGC | | |
| | | | ACATCCATTTCATTGGCAAGATCC | | |
| | | | AGGGCAGCGACGCCGGCTGGACTT | | |
| | | | TGGGCTACATGCTGAACCTGAC | | |
| | | | CAACATGATCCCAGCTGAGCAACC | | |
| | | | ATTGTCCACACCTCTCTCCCACTCC | | |
| | | | ACCTATGTCTTCCTCATGGTT | | |
| | | | CTATTCTCCCTGGTCCTTTTCACAG | | |
| | | | TGGCCATCATAGGCTTGCTTATCTT | | |
| | | | TCACAAGCCTTCATATTTCT | | |
| | | | GGAAAGATATGGTATAGCAAAAGC | | |
| | | | AGCTGAAATATGCTGGCTGGAGTG | | |
| | | | AGGAAAAAAATCGTCCAGGGAG | | |
| | | | CATTTTCCTCCATCGCAGTGTTCAA | | |
| | | | GGCCATCCTTCCCTGTCTGCCAGG | | |
| | | | GCCAGTCTTGACCAGTGTGAA | | |
| | | | GCTTCCTTGGCTTTTACTGAAGCCT | | |
| | | | TTCTTTTGGAGGTATTCAATATCCT | | |
| | | | TTGCCTCAAGGACTTCGGCA | | |
| | | | GATACTGTCTCTTTCATGAGTTTTT | | |
| | | | CCCAGCTACACCTTTCTCCTTTGTA | | |
| | | | CTTTGTGCTTGTATAGGTTT | | |
| | | | TAAAGACCTGACACCTTTCATAAT | | |
| | | | CTTTGCTTTATAAAAGAACAATATT | | |
| | | | GACTTTCTCTAGAAGAACTGA | | |
| | | | GAGTCTTGAGTCCTGTGATAGGAG | | |
| | | | GCTGAGCTGGCTGAAAGAAGAATC | | |
| | | | TCAGGAACTGGTTCAGTTGTAC | | |
| | | | TCTTTAAGAACCCCTTTCTCTCTCC | | |
| | | | TGTTTGCCATCCATTAAGAAAGCC | | |
| | | | ATATGATGCCTTTGGAGAAGG | | |
| | | | CAGACACACATTCCATTCCCAGCC | | |
| | | | TGCTCTGTGGGTAGGAGAATTTTCT | | |
| | | | ACAGTACGCAAATATGTGCTA | | |
| | | | AAGCCAAAGAGTTTTATAAGGAAA | | |
| | | | TATATGTGCTCATGCAGTCAATAC | | |
| | | | AGTTCTCAATCCCACCCAAAGC | | |
| | | | AGGTATGTCAATAAATCACATATT | | |
| | | | CCTAGGTGATACCCAAATGCTACA | | |
| | | | GAGTGGAACACTCAGACCTGAG | | |
| | | | ATTTGCAAAAAGCAGATGTAAATA | | |
| | | | TATGCATTCAAACATCAGGGCTTA | | |
| | | | CTATGAGGTAGGTGGTATATAC | | |
| | | | ATGTCACAAATAAAAATACAGTTA | | |
| | | | CAACTCAGGGTCACAAAAAATGCA | | |
| | | | TCTTCCAATGCATATTTTTATT | | |
| | | | ATGGTAAAATATACATAAATATAA | | |
| | | | TTCACCATTTTAACATTTAATTCAT | | |
| | | | ATTAAATACGTACAAATCAGT | | |
| | | | GACATTTAGTACATTCACAGTGTT | | |
| | | | GTGCCACCATCACCACTATTTAGTT | | |
| | | | CCAGAACATTTGCATCATCAA | | |
| | | | TACATTGTCTAGAGACAAGACTAT | | |
| | | | CCTGGGTAGGCAGAAACCATAGAT | | |
| | | | CTTTTGTGTTTACAGCTATGGA | | |
| | | | AACCAACTGTACCATAAAGATAGT | | |
| | | | TCACTGAGTTTTAAAGCCAACCCA | | |
| | | | CATCTTATTTTTCCAAGGTTTA | | |
| | | | ATTTAGTGAGAGGGCAGCATTAGT | | |
| | | | GTGGAGTGGCATGCTTTTGCCCTAT | | |
| | | | CGTGGAATTTACACATCAGAA | | |
| | | | TGTGCAGGATCCAAGTCTGAAAGT | | |
| | | | GTTGCCACCCGTCACACAACATGG | | |
| | | | GCTTTGTTTGCTTATTCCATGA | | |
| | | | AGCAGCAGCTATAGACCTTACCAT | | |
| | | | CGAAACATGAAGAGACCCTGCACC | | |
| | | | CCTTTCCTTAAGGATTGCTGCA | | |
| | | | AGAGTTACCTGTTGAGCAGGATTG | | |
| | | | ACTGGTGATGTTTCATTCTGACCTT | | |
| | | | GTCCCAAGCTCTCCATCTCTA | | |
| | | | GATCTGGGGACTGACTGTTGAGCT | | |
| | | | GATGGGGAAAGAAAAGCTCTCACA | | |
| | | | CAAACCGGAACCCAAATGTCCC | | |
| | | | CTATCTCTTGAATGATCAAGTCACT | | |
| | | | TTTCACAACATCCAGGTGAATATA | | |
| | | | AAAACTTAATAAAGCTGTGGA | | |
| | | | AAGGAACTCTTAATCTTCTTTTCTG | | |
| | | | CTACTTAGGTTAAATTCACTAGATC | | |
| | | | TTGATTAGGAATCAAAATTC | | |
| | | | GAATTGGGACATGTTCAAATTCTTT | | |
| | | | CTTGTGGTAGTTGCCTATACTGTCA | | |
| | | | TCGCTGCTGTTGGTTGAGCA | | |
| | | | TTTGTGGTGTACCACGCTGTGTGCT | | |
| | | | CAAGGGTATTACATTCATCTTCTCA | | |
| | | | TTTAATCCTCACAACAATCT | | |
| | | | CAAGAAGGTAGGTATTACAATTCC | | |
| | | | CACTTCATAGAAACAGAAACTGAG | | |
| | | | GTTCAGAGAGGTTAAGTCATTT | | |
| | | | GCCCAAATGGCTGAGCCAAAGCCT | | |
| | | | ACCATGTACCTAACCTTTATTTTCT | | |
| | | | TTCCCGAACATACCAGGCTGT | | |
| | | | CTCCTCATAACTTCCAAGCATGCA | | |
| | | | CTTAAAACTCCACATGAATACAAG | | |
| | | | GTTCATGGGACTTGGTATTCAT | | |
| | | | AGAAAGGGAGGCAGAAAGCTGGT | | |
| | | | CTGTTCCTGATAGGCTTGTAATTTA | | |
| | | | ATATCATTCTGTTCATGTGCTT | | |
| | | | TGGATGGAAGCACATCTGGCATAT | | |
| | | | GATGCTAATCAGTGGTTCCCATAC | | |
| | | | CCCTGGCTTCCTAATTTTAATG | | |
| | | | TTTGCTCACAGCATAGTAGATTGA | | |
| | | | CATCAAATAGTGGCCGATGATGAT | | |
| | | | GAAAATAAAGGTCAAATAAGTT | | |
| | | | GAGCCAATAACAGCCGCTTTTTTC | | |
| | | | CTTCTGTCTGCGTATACAAAGCACT | | |
| | | | GTCATGCACACAATCTATTCT | | |
| | | | GACCCTCACAACAACCCATAAGGG | | |
| | | | TGTAAATAGTATTTCCATTTTACAA | | |
| | | | ATGAGGATCACACAAACTACT | | |
| | | | ACATGGCAGAGCAGATACTCCAAC | | |
| | | | TCATGTCTTCTGGTTGAAGCCTATT | | |
| | | | GCTTTTTCTTTTCTAAACACT | | |
| | | | TTCCCTCAGCAAGTTGGAATTAGA | | |
| | | | CTTCACAAGTCTCCTTCAGAGAAC | | |
| | | | ACAAATCTTTTCTTATTCCATT | | |
| | | | CCTGTTTGGTTGCCTACGTCCAATC | | |
| | | | TCCCCCTCCCCAGAGATGCCAAAA | | |
| | | | AAAAAATCCTTTAAGGTATTT | | |
| | | | GGGACCCAAACTCAACTTGTTAAA | | |
| | | | ATCTCAAATTATGGAGACAATCAG | | |
| | | | CAGACACAACCTAACCCCAATT | | |
| | | | ATTTTGGCAGGAAGGTTGGTTTAG | | |
| | | | AGGCAGATCCAGCAATCTGCTTTG | | |
| | | | GGCCACTCTGGGTGGGGTAGGT | | |
| | | | GAAATAAGATTGGTCACTGTTAAC | | |
| | | | TAATTTTAATATTGGATTGGCCATT | | |
| | | | GGTTATCACTGATTACCATTC | | |
| | | | TCCCCTGGATTTTCACCCAGGACTC | | |
| | | | AAAACTTGGTTCTGCTAACCCTGTT | | |
| | | | CCTTTATGAGGAACCTTTTA | | |
| | | | AAGATTCCTTTATAAGGTGGGAGT | | |
| | | | TTTTTTTCTATGAACCTATAGGGGA | | |
| | | | GAAAAAAGATCAGCAGAAGTC | | |
| | | | ATTACTTTTTTTTTTTTTTTTTTTTTT | | |
| | | | TTTTGAGAGAGACTCTCACTCCATT | | |
| | | | GCCCAGGCTGGACTGCAG | | |
| | | | TGGTGCTATCTCGGCTCACTGCAA | | |
| | | | CCTCCGCCTCCTGGGTTCAAGCAA | | |
| | | | TTCTCCTGCCTCAGCCTCCCGA | | |
| | | | GTAGCTGGGATTGCAGGTGCCCAC | | |
| | | | CACCACACCCGGCTAATTTTTGTAT | | |
| | | | TTTTAGTAAAGACAGGGTTTC | | |
| | | | ACCATGTTGGCCAGGCTGGTCTCC | | |
| | | | AACTCCCAATCTCAGGTGATCCTA | | |
| | | | TTGCCTCGGGCTCCCAAAGTGC | | |
| | | | TGGGATTACAGGAGTGAGCCACCA | | |
| | | | TGCCTGGCCAGAAGTGGTTACTTC | | |
| | | | TGTAGACAAAAGAATAATGCTA | | |
| | | | CTTAATCAGGCTTTCTGTGTGACAA | | |
| | | | GAAAGAGAAAGAAAATAAAGAAG | | |
| | | | TTTCAATTCATCCAATTCTTAA | | |
| | | | TAAGAAATATGTAAATAAAATTTT | | |
| | | | TTAAAATTACACTTCATTTTAATGT | | |
| | | | TGTATCAGTCAAGGTCCCTGC | | |
| | | | AAGAGATGGATGGTATGGTACACT | | |
| | | | CAAACTGGGTAACACAGGAGAGTT | | |
| | | | TTCAGAAAGCAACTAAATCCAA | | |
| | | | AATACTATCAAGGAATCAATATAA | | |
| | | | AAATTGTTAATATTTTTCTCATACT | | |
| | | | AAATTTTCAAAATATTTTGTG | | |
| | | | TCTATTACATTTACAGCACATCTTA | | |
| | | | ATTAGGACTAGCTGTGTGTTCACCT | | |
| | | | CACATGTGGCTTGTAGCTAC | | |
| | | | CATACTGGACAGCACATGTCCAAA | | |
| | | | AAAATACACGTAAAGTTAAAGTTT | | |
| | | | AAAAGACACAGGAACTAAGCCC | | |
| | | | TCATTGTCTTTCCCTTGGGAGGTAG | | |
| | | | TTTAAAGAGCTATAGATGCTGTAA | | |
| | | | CATTCTTGCTATTATTTATTA | | |
| | | | TATATGACATTATTCCTAAAAAAG | | |
| | | | CTTTTGAGATCCTAGGTTGTATTCC | | |
| | | | TCAGGTTTTGTTGCCTTCCCA | | |
| | | | TGAAGATGTGAAGGCAGGGATGCC | | |
| | | | TGTTATTCAGTCCAAGATGCATGA | | |
| | | | CAAGAGACCTTGGGAAAGTTTC | | |
| | | | ATCTGGATTTAAAGATTAATTCTTG | | |
| | | | ATGCTTACATTCCATACTCAAAAT | | |
| | | | GTAAATTTCAATATTAAAATA | | |
| | | | AAGATGATTTTTTTTTTGGAGCTAG | | |
| | | | TCTTGCTCTGTTGCCCAGGCTGGAA | | |
| | | | TGCAGTGGCATGATCATGGC | | |
| | | | TCACTGCAGCCTCGACCTCCCAAG | | |
| | | | CTCAAGCAAGGCTACAGGTGTGCA | | |
| | | | CCTAAGTAGCTAGGACTACAGG | | |
| | | | TGTGCACCACCATGTCTAGCTATTT | | |
| | | | TTTTTTCTGTAGAGACAGGGTTTTC | | |
| | | | CTATGTTGTCCAGGCTGGTC | | |
| | | | TCGAACTCCTGCCCTCAAGCAATC | | |
| | | | CTCCTGCCTTGGCCTCCCAAAGTGT | | |
| | | | TGAGATTACAGGCGTAAGCCA | | |
| | | | CTGCACCTGGCCAAGATGAATATT | | |
| | | | TTAATAGCTCACAGAACAAAGTTT | | |
| | | | GCCACATAATGATAAAATTACT | | |
| | | | ATGAAAATATATTCCCTTTATTGTC | | |
| | | | AGTTTAAAAGATCAACTGAGTTTC | | |
| | | | ACCCAAACTGGTCTGGCCCCT | | |
| | | | CTCTGATTCAAATACCAATAGTTG | | |
| | | | CTCTGATTCAAATTCCAACTGTTAG | | |
| | | | AACATGACAGCTGCTCATAAC | | |
| | | | TAGCTTTGCTTACTAACCATGTTTC | | |
| | | | TTTCCATTTGTATTAGGTCGTTTAC | | |
| | | | TTTTTATAACAGCCTCAAAG | | |
| | | | TTTCATGAATTGCTGCAGTAAACA | | |
| | | | TTGATTTTCATGTTTGTGAGTCTGC | | |
| | | | AAGCCAGCTGGGCAGCTCTAC | | |
| | | | TTCAGGTGGTAAGGGTGGATCAGA | | |
| | | | CCTATTCCATATACCTCTTGTTCTC | | |
| | | | CTTGTCCAGTGGTTTCTAGGG | | |
| | | | ATATGTTCTCATGATGAACCCCGC | | |
| | | | AGAGGCTCGTGAAAGTGAGAGGA | | |
| | | | AACTAGGATGCCTCTTAAGGTCT | | |
| | | | TGGTCAGGATGGGGTCTCCTGTCA | | |
| | | | CTTCTGTCACAGGCTATTGTAAGTC | | |
| | | | ATATGAGCAACCTCAATAAAA | | |
| | | | TATAAACAAGTCAGATAAACAGTG | | |
| | | | GGAGGAATGGCAAAGTCATATGGC | | |
| | | | CAAGGCCATGAGTGATTAATTT | | |
| | | | TAACACAGGAAAAAAGTAAAGCA | | |
| | | | TTAAATGCGATTATTTAATATACA | | |
| | | | ATGTCTTATTAACTGAAATATAA | | |
| | | | AATGTGTTTACTGTAAAATATAAT | | |
| | | | CTGTTTATCTCACCAAAGAAATATT | | |
| | | | ATCTTTAAAAAATGTCATTAC | | |
| | | | TTCTAAGACATCATCAGTCTGCAA | | |
| | | | CTTCTTTCCATAGCCTTAATCAGGA | | |
| | | | TGCTGTGGCAGCTCCCACATT | | |
| | | | AGCCTCGCATTCTAAACTGGTAGA | | |
| | | | TGTCCTAGGAAACCATACATCTAT | | |
| | | | GTATTTTTCTTATTTTATACGT | | |
| | | | TTAGGACAATGTATAGCTAATTAC | | |
| | | | CCAACTTTTTATTTGCATACAAATC | | |
| | | | TAATACAACTGAACACAATCA | | |
| | | | GTTTTATCACAGGTATAATGGATTT | | |
| | | | TTCAATAGTGAGGAGGTGCCTCCA | | |
| | | | TGAGCCTTCTCTTTAGAAAAG | | |
| | | | TGGCATTCAAGACTCTTCATTTGAA | | |
| | | | GTGAAGATTGCTATGTCTTTTGCAT | | |
| | | | TGCTCTATTTTACATAAATT | | |
| | | | AAGTTATAAATTGACACTATAATC | | |
| | | | AACTGACACCATGATCAGTGATGA | | |
| | | | TGATCACCCTCATCAGCACTAG | | |
| | | | AGTTGACTTGTTTTTATAACCCCTT | | |
| | | | TGCATGTATGTTGAATAGCAAAGT | | |
| | | | TCATCAGAGAACATGTATTAG | | |
| | | | TCAATGGTAAGTAAGATACTCTCA | | |
| | | | TCTAAGAAATAACATCACCTCTTCT | | |
| | | | AATGAAGTTCTAAGAAGAGAG | | |
| | | | GGAAGAAAAAGTCTTGGGAGCTAG | | |
| | | | TCAGGGAATAGTGTGTATTTGCAA | | |
| | | | TTACCTAAACTGAACTCTACCA | | |
| | | | TTACTCCTAACCCAGTTCCTCCTCC | | |
| | | | TGTGTTTTACATGATTAATGCCACC | | |
| | | | CCTGCCTCAATGAACCAAGA | | |
| | | | TCAGCTCCATCACTGGGACCTCCC | | |
| | | | CATTCTGCCTGTGCAATATTTTTCT | | |
| | | | TTTTTATTTCTCCTTCTAATA | | |
| | | | TTACTGTTATTGCTCCAGTAAAGA | | |
| | | | GCTGTAATATATTTTACCTGGACTG | | |
| | | | ATACCAGGAATGGTGGTGTTG | | |
| | | | CTTCCAATCTGTTGCTGCTAGATTA | | |
| | | | ATCTTTGCAAAGCACAGGCTTAAT | | |
| | | | TTCATTGCTGCTCAACTAAAA | | |
| | | | CCACTGGTGGCTTTCCATTGCCTAC | | |
| | | | AAAATAAAGTCAACCTCCCCATCA | | |
| | | | GACATTCAAGGCTTTCAATGA | | |
| | | | TCCATGGCCGCCAGCTCTCTCCAG | | |
| | | | GCTCATATCCCACTCCACTCCTCTG | | |
| | | | ATGTTTCCTACACTACACTAC | | |
| | | | ACTATACTACACTACAGCCAGGTA | | |
| | | | GAATGACTGTTCACCCAACACCAC | | |
| | | | TCAGGTTGTCTTCTCAACTTGG | | |
| | | | AATACTCTTGCACCTTCAAAGCTC | | |
| | | | ATTTCAAATGCCCCTTCATTTGTGA | | |
| | | | AGCCTTCTTCTCCAAATTTCCAAG | | |
| | | | TCAGAATGTCTCTTCCTTGTGCTAC | | |
| | | | CACAACCCTTTAACTGAGCCTCCA | | |
| | | | TTAGTGCACTGAGACCATTCT | | |
| | | | GTTCAGTGTCTGGGTGAAGCTTCCT | | |
| | | | GGTGAAAAATATGTTACCTATTTCT | | |
| | | | TTCTGAAAAGTTGGATTCAG | | |
| | | | GGATATTATCACGGACCTAAGGTA | | |
| | | | ATAGTTCTAGCCAACCTCCCTGTCC | | |
| | | | ACTGCCAGGCCGACTACAAAC | | |
| | | | CCTTCTGTTGCTGGCGAGCTGGTCC | | |
| | | | GCACCACTAGTTCTGCTTCACTCTA | | |
| | | | TTTATCTCTTGATGTAACCA | | |
| | | | TCTTCTTTCTCCAGGTTTTAAGAAC | | |
| | | | CAGCCCAACTCCTGGTTCCCTGAT | | |
| | | | GAAGCTTTTATTCCCCTAGCC | | |
| | | | ACATGGAACTTTTCCTTTTTGGAAC | | |
| | | | ATGCCTTTAGTTTCTGTGTAGTTTG | | |
| | | | CCATGCAGCACTTCATTGTA | | |
| | | | CACATTATTAAAACAGAATTTTAA | | |
| | | | GGATTAGAATGAACCTTAAAAGAT | | |
| | | | CATGCATCTCAAAATTTAATGT | | |
| | | | ACATACAAATTACCCAGGGATTTT | | |
| | | | GTTGAAATAAAAATTATTTAATTTT | | |
| | | | AATTAATATAAATAATTCAGT | | |
| | | | AGGTCTGGGGTGAGGCCTGAGGTT | | |
| | | | TTACATTTCCAACAAGCTGCCAGG | | |
| | | | TAAAGCCAATACATCTGTCCAG | | |
| | | | GAATCACACTTTGCGTATCAAAGG | | |
| | | | TCTAGATGACATTATCATTCCAAA | | |
| | | | GAGTTTCTTTTACAGGCTCTCA | | |
| | | | GATCAGTGTTCATCCACTACCTGA | | |
| | | | CTACTGTCATTCACAGGCATTCTGT | | |
| | | | TCCACAGCAGGCCAGCTAACG | | |
| | | | TGGTATTTACAAAGCTCACTCCTCT | | |
| | | | TATACAACAATCCAAGTGTTTCTTT | | |
| | | | TGTCAGTTGTCTGTGCCCCA | | |
| | | | GGAGATCCCTCTCTGCCTTGCCTTG | | |
| | | | CCCTCTGCCTTTGGAGACCAGCAC | | |
| | | | CTCATACTCAGTGAAGGCCTG | | |
| | | | GAGTGCTTAAGAGGGATTTCTTCC | | |
| | | | AGCTCTCTTGCCCTGGTCTTCAGTG | | |
| | | | TATTAGATGTATTACCTCCAT | | |
| | | | GCTCTCAGTAGAGGCCCATAGGAA | | |
| | | | AGAGTAGGTAGGTTATGCCAGCTC | | |
| | | | ACACGCATCCTTTAAAAATGGT | | |
| | | | TTAGAAGTTTAGCTGGTTTCTTATT | | |
| | | | TGTCACTCTACTAGGGATGA | | |
| | | | AACAGCTAATCATGTTCAATAGTT | | |
| | | | ACATTTAGATTGGTTTTTAAAAACT | | |
| | | | ATGATTGTATTAGTTCGTTTC | | |
| | | | CATGCTGCTGATAAAGACATATCT | | |
| | | | GAGACTGGAAACAAAAAGGGTTTA | | |
| | | | ATTGGACTTACAGTTCCACATG | | |
| | | | GCTGGGGAGGCCTCAAAATCAGGT | | |
| | | | GGGAGGCAAAAGGTACTTCTTACG | | |
| | | | TGGTGGCATCAAGAGCAAAATG | | |
| | | | AGGAAGAACCAAAAGCAGAAACT | | |
| | | | CTTCATAAACCCACCAGATCTTGT | | |
| | | | GGGACTTATTATCACGAGAATAG | | |
| | | | CACAGAAAAGACTGGCCTCCATGA | | |
| | | | TTCAATTACCTCCCACTGCGTCCCT | | |
| | | | CCCACAACATGTGGGAATTCT | | |
| | | | GGGAGATACAATTCAAGTTGAGAT | | |
| | | | TTGGGTGGGGACACAGCCAAACCA | | |
| | | | TATCATTCCTCCCTGGGCTCCT | | |
| | | | CCAAATTTCATAATCCTCACATTTC | | |
| | | | AAAACCAATCATTCCTTCCCAACA | | |
| | | | GTTCCCCAAAGTCTTAACTCA | | |
| | | | TTTCAGCATTAACCCAAAAGTCCA | | |
| | | | CAGTCCAAAGTCTCATCTGAGACA | | |
| | | | AGGCAAGTCCCTTCCACTTACA | | |
| | | | AGCCTGTAAAAGCAAGCTAGTTAC | | |
| | | | CTCCTAGATACAATGGGGGGTACA | | |
| | | | GGTATTGGGTAAATACAGCTGT | | |
| | | | TCCAAATGAGAGAAATTGGCCAAA | | |
| | | | ACAAAGGGGTTACAGGGTCCATGC | | |
| | | | AAGTCTGAAATCCAGTGGGGCA | | |
| | | | GTCAAATTTTAAAGCTCCATAATG | | |
| | | | ATCTCCTTTGACTCCATGTCTCACA | | |
| | | | TTCAGGTCATGCTGATGCAAG | | |
| | | | AGATAGGTTCCCATGGTCTTGTGC | | |
| | | | AGCTCCGCCCCTGTGGCTTTGCAG | | |
| | | | AGTACAGCCTCCCTCCTGGCTG | | |
| | | | CTTTCTCAGGCTGATGTTGAGTGTC | | |
| | | | TGTAGCTTTTCCAGGCACAAGATG | | |
| | | | CAAGTTGGTGGTTGATCTACC | | |
| | | | ATTCTGGGGTCTACCATTCTGGGGT | | |
| | | | CTACCGTTCTGGGACTGTGGCCTTC | | |
| | | | TTCTCACAGCTCCACTAGGC | | |
| | | | AGTGCCCCAACAGGGACTCTGTGT | | |
| | | | GGGGGCTCTGCCCCACATTTCCCTT | | |
| | | | CCACACTGCCCTAGGAGAGGT | | |
| | | | TCCCCATGAGGGCTCTGCCCCTGC | | |
| | | | AGCAAACTTTTGCCTGGACATCCA | | |
| | | | GGTGTTTCCATATATATTCTGA | | |
| | | | AATCTAGGCAGAGGTTCCCAAATC | | |
| | | | TCAATTCTTGACATCTCTGCACCCA | | |
| | | | CAGGCTCAACATCACATGGAA | | |
| | | | GCTGCCAATGCTTGGGGCCTCTAC | | |
| | | | CCTCTGAAGCCACAGCCCAAGCTC | | |
| | | | TATGTTGGCTCCTTTCAGCCAT | | |
| | | | GGCTGGAGCAGCTGGGACACAGG | | |
| | | | GCACCAAGTCCCTAGGCTGCACAC | | |
| | | | AGCACAGAGACCCTGGGCCCAGC | | |
| | | | CCACAAAACCACTTTTTCCTCCTGG | | |
| | | | GCCTCTGGGCCTGTGATGGGAGGG | | |
| | | | GCTGCCATGAAGGTCTCTGAC | | |
| | | | ATGACCTGGAGACATTTTCCCCAT | | |
| | | | GGTCTTGGGGATTAACATTAGGCT | | |
| | | | CCTTGCTGCTTATGCAAATTTC | | |
| | | | TGCAGCCAGCTTGAATTTCTCCTTA | | |
| | | | AAAAAAATGGGTTTTTCTTTTCTAC | | |
| | | | TGCATCATCAGGCTGCAGAT | | |
| | | | TTTCCACATTTATGCTCTTGTTTCC | | |
| | | | CTTTTAAAACAGAATGTTTTTAACA | | |
| | | | GCACCCAAGTCACCTTTTCA | | |
| | | | ATGCTTTGCTGCTTAGAAATTTATT | | |
| | | | CCACCAGATACCCTAAGTCATCTC | | |
| | | | TCTCAAGCTCTAAGTTCCACA | | |
| | | | AATCTCTAGGGCAAGGGTCAAATG | | |
| | | | CTGCCAGTCTCCTTGCTAAAACAT | | |
| | | | AACAAGGGTCACCTTTACTTCA | | |
| | | | GTTCCCAACAAGGTCTTCATCTCC | | |
| | | | ATCTGAGACCACCTCAGCCTGGAC | | |
| | | | CTTATTGTTCATATCACTATCA | | |
| | | | GTATTTTTGTCAATGCCATTCACAG | | |
| | | | TCTCTAGGAGGTTCCAAACTTTCCT | | |
| | | | ACATTTTCCTATCTTCTTCT | | |
| | | | GAGCCCTCCAGATTATTTCAACAC | | |
| | | | CCAGTTCCAAAGTTGCTTCCACATT | | |
| | | | TTCGGGTATCTTTTCAGCAAT | | |
| | | | GCCCCACTCTACTGGTACTATTAGT | | |
| | | | CCATTTTCATGCTGCTGATAAAGA | | |
| | | | CATACCTGAGACTGGGAACAA | | |
| | | | AAAGAGGTTTAATTGGACTTATAG | | |
| | | | TTCCACCTGGCTGGGGAGGCCTCA | | |
| | | | GAATCATGGCAGGAGGTGAAAG | | |
| | | | GCATTTCTTACACGGCAGCAGCAA | | |
| | | | GAGAAAAATGAAGAAGCAGCAAA | | |
| | | | AGCAGAAACCCCTGATAAAACCA | | |
| | | | TCAGATCTCGTGAGACTTATTCACT | | |
| | | | ATCACAAGAATAGCATGGGAAAG | | |
| | | | ACCAGCCCCCTTGATTCAATTA | | |
| | | | CCTCCCCCTGGGTCCTGTGGGAAT | | |
| | | | TCTGGAAGGTACAATTCAAGTTGA | | |
| | | | GATTTGGGTGGGGACACAGCCA | | |
| | | | AACCATATCAATGATTTTCTACTTT | | |
| | | | AACCAGCTGAATGGAAGTACAATC | | |
| | | | TCTTGCTATATGACACAATAA | | |
| | | | TTATTTGCAAAATGAGTAAACATA | | |
| | | | TCATAAGGAAATTATTTTTACAAG | | |
| | | | GTTTGAAACCTGAAATGCAGTC | | |
| | | | TATTATCATACATAACTAAAAATA | | |
| | | | GAGCCTCAATAAACAGATTCCCAG | | |
| | | | TTTTGAAAATGCAACATTTGTA | | |
| | | | CTCCACATTGTCAGTTTTCTTAGCT | | |
| | | | ATATTTATAAATACTCCTATAAAA | | |
| | | | ATGTAAAGAAACACATAATGT | | |
| | | | AGATTGCTAATTTTATAATAACAC | | |
| | | | AAGTTGATTTTGACATCCAACTTAT | | |
| | | | TAATTATGAAATGACTTTTGG | | |
| | | | CCTAGTAACAATGAAAATGGGGGC | | |
| | | | AAATACAGATAAATGGTAATTCTT | | |
| | | | AGAATGAACTACTCAGCACCAA | | |
| | | | TTCTAAGTTTTTCTTGATGGTAAAT | | |
| | | | CATAATGTTCCCTTTCTCCTCGGTT | | |
| | | | CTGCAATCTATAGGCATACC | | |
| | | | ATAATTGTAATCAATAGCTTAAAA | | |
| | | | ATATGTCTCTCTGTCCTATTCTGTA | | |
| | | | TCTGTATCTCTTGGATTTTTA | | |
| | | | CCTTTGCAATAGTCAACTGAACCA | | |
| | | | TCTTCTTGGAGTACTCATGAAGAT | | |
| | | | GGAAGTCTACATGGAGAATACA | | |
| | | | GGATGAATCCACTCTGTCTCCTGC | | |
| | | | AGTGAAGTCTGTTTGAAGGATGTA | | |
| | | | TTTGGCTGTCTTCTGGACAGGC | | |
| | | | CATTCTAATAACAGAAACAAACAA | | |
| | | | GTTATTTTAAAACTTATTGGAATAT | | |
| | | | TCAAATATTAACCAAAGTAGA | | |
| | | | AAAATATAATACACATCCATGTGC | | |
| | | | CCATCACAGAACTTCACTGATTAT | | |
| | | | CATCATTTAGCCAGTCTTGAAG | | |
| | | | AAGCAAGTGCTAATTACAATCACA | | |
| | | | AATGAAACAAGATTCAGACTTCAT | | |
| | | | GAAGAGCACTGCGCTATAATAA | | |
| | | | AAGAAGAAATGAGCACATACATTC | | |
| | | | TTTTACTGACAGTCAAATGGTGAA | | |
| | | | GGTGGGCAGAATCATTATGTGA | | |
| | | | TGCAACATGGCAAAAGTATACAGA | | |
| | | | CAGTGCATCCAGAGGAAGGCACCT | | |
| | | | TGCTGAATGACTAGAATGGAAG | | |
| | | | TAGGAGACATTTTGCAGGCCCCCT | | |
| | | | TCATCCTGCAGGCAGAACCAGAAC | | |
| | | | CACAGCAGCTCTATTTGCCTAT | | |
| | | | TCCTCTTTAAATTACAAAGTTAAA | | |
| | | | ATTTGGGAGTAGTAGAAAATCAAT | | |
| | | | TGGTTATCTTATAGAGTCTCCT | | |
| | | | AGAATATTTCATTGGCATTGAGAA | | |
| | | | GGTGGAAAATGCAAATTATATACT | | |
| | | | TTAAAATGTAATTTTTGCTTTT | | |
| | | | CACATATGCTTAAAGCCTAAAACC | | |
| | | | TCTTAATAAACTTCTTCTGAAATAT | | |
| | | | A (SEQ ID NO: 39) | | |
| | | NM_001164181.1 | CCTGTTGCTCTTTGCTCTAATGAGC | NP_001157653.1 | MERAREVIPRSQHQETP |
| | | | CTTGAGAAAGGATTGCTGGTCATG | | VYLGATAGMRLLRMES |
| | | | GGACCAGAGGCTTTATGGGGA | | EELADRVLDVV |
| | | | GGGAAGAACTGTTCTTGACTTTCA | | ERSLSNYPFDFQGARIIT |
| | | | GTTTTTCGAGCGGGTTTCAAGTATG | | GQEEGAYGWITINYLLG |
| | | | GGATTGTGCTGGATGCGGGTT | | KFSQKTRWFSIVPYETN |
| | | | CTTCTCACACAAGTTTATACATCTA | | NQETFG |
| | | | TAAGTGGCCAGCAGAAAAGGAGA | | ALDLGGASTQVTFVPQ |
| | | | ATGACACAGGCGTGGTGCATCA | | NQTIESPDNALQFRLYG |
| | | | AGTAGAAGAATGCAGGGTTAAAG | | KDYNVYTHSFLCYGKD |
| | | | GTCCTGGAATCTCAAAATTTGTTCA | | QALWQKLAK |
| | | | GAAAGTAAATGAAATAGGCATT | | DIQVASNEILRDPCFHPG |
| | | | TACCTGACTGATTGCATGGAAAGA | | YKKVVNVSDLYKTPCT |
| | | | GCTAGGGAAGTGATTCCAAGGTCC | | KRFEMTLPFQQFEIQGIG |
| | | | CAGCACCAAGAGACACCCGTTT | | NYQQCH |
| | | | ACCTGGGAGCCACGCCAGGCATGC | | QSILELFNTSYCPYSQCA |
| | | | CGTTGCTCAGGATGGAAAGTGAAG | | FNGIFLPPLQGDFGAFSA |
| | | | AGTTGGCAGACAGGGTTCTGGA | | FYFVMKFLNLTSEKVSQ |
| | | | TGTGGTGGAGAGGAGCCTCAGCAA | | EKVTE |
| | | | CTACCCCTTTGACTTCCAGGGTGCC | | MMKKFCAQPWEEIKTS |
| | | | AGGATCATTACTGGCCAAGAG | | YAGVKEKYLSEYCFSG |
| | | | GAAGGTGCCTATGGCTGGATTACT | | TYILSLLLQGYHFTADS |
| | | | ATCAACTATCTGCTGGGCAAATTC | | WEHIHFIGK |
| | | | AGTCAGAAAACAAGGTGGTTCA | | IQGSDAGWTLGYMLNL |
| | | | GCATAGTCCCATATGAAACCAATA | | TNMIPAEQPLSTPLSHST |
| | | | ATCAGGAAACCTTTGGAGCTTTGG | | YVFLMVLFSLVLFTVAII |
| | | | ACCTTGGGGGAGCCTCTACACA | | GLLIFH |
| | | | AGTCACTTTTGTACCCCAAAACCA | | KPSYFWKDMV (SEQ ID |
| | | | GACTATCGAGTCCCCAGATAATGC | | NO: 42) |
| | | | TCTGCAATTTCGCCTCTATGGC | | |
| | | | AAGGACTACAATGTCTACACACAT | | |
| | | | AGCTTCTTGTGCTATGGGAAGGAT | | |
| | | | CAGGCACTCTGGCAGAAACTGG | | |
| | | | CCAAGGACATTCAGGTTGCAAGTA | | |
| | | | ATGAAATTCTCAGGGACCCATGCT | | |
| | | | TTCATCCTGGATATAAGAAGGT | | |
| | | | AGTGAACGTAAGTGACCTTTACAA | | |
| | | | GACCCCCTGCACCAAGAGATTTGA | | |
| | | | GATGACTCTTCCATTCCAGCAG | | |
| | | | TTTGAAATCCAGGGTATTGGAAAC | | |
| | | | TATCAACAATGCCATCAAAGCATC | | |
| | | | CTGGAGCTCTTCAACACCAGTT | | |
| | | | ACTGCCCTTACTCCCAGTGTGCCTT | | |
| | | | CAATGGGATTTTCTTGCCACCACTC | | |
| | | | CAGGGGGATTTTGGGGCATT | | |
| | | | TTCAGCTTTTTACTTTGTGATGAAG | | |
| | | | TTTTTAAACTTGACATCAGAGAAA | | |
| | | | GTCTCTCAGGAAAAGGTGACT | | |
| | | | GAGATGATGAAAAAGTTCTGTGCT | | |
| | | | CAGCCTTGGGAGGAGATAAAAACA | | |
| | | | TCTTACGCTGGAGTAAAGGAGA | | |
| | | | AGTACCTGAGTGAATACTGCTTTTC | | |
| | | | TGGTACCTACATTCTCTCCCTCCTT | | |
| | | | CTGCAAGGCTATCATTTCAC | | |
| | | | AGCTGATTCCTGGGAGCACATCCA | | |
| | | | TTTCATTGGCAAGATCCAGGGCAG | | |
| | | | CGACGCCGGCTGGACTTTGGGC | | |
| | | | TACATGCTGAACCTGACCAACATG | | |
| | | | ATCCCAGCTGAGCAACCATTGTCC | | |
| | | | ACACCTCTCTCCCACTCCACCT | | |
| | | | ATGTCTTCCTCATGGTTCTATTCTC | | |
| | | | CCTGGTCCTTTTCACAGTGGCCATC | | |
| | | | ATAGGCTTGCTTATCTTTCA | | |
| | | | CAAGCCTTCATATTTCTGGAAAGA | | |
| | | | TATGGTATAGCAAAAGCAGCTGAA | | |
| | | | ATATGCTGGCTGGAGTGAGGAA | | |
| | | | AAAAATCGTCCAGGGAGCATTTTC | | |
| | | | CTCCATCGCAGTGTTCAAGGCCAT | | |
| | | | CCTTCCCTGTCTGCCAGGGCCA | | |
| | | | GTCTTGACGAGTGTGAAGCTTCCTT | | |
| | | | GGCTTTTACTGAAGCCTTTCTTTTG | | |
| | | | GAGGTATTCAATATCCTTTG | | |
| | | | CCTCAAGGACTTCGGCAGATACTG | | |
| | | | TCTCTTTCATGAGTTTTTCCCAGCT | | |
| | | | ACACCTTTCTCCTTTGTACTT | | |
| | | | TGTGCTTGTATAGGTTTTAAAGACC | | |
| | | | TGACACCTTTCATAATCTTTGCTTT | | |
| | | | ATAAAAGAACAATATTGACT | | |
| | | | TTGTCTAGAAGAACTGAGAGTCTT | | |
| | | | GAGTCCTGTGATAGGAGGCTGAGC | | |
| | | | TGGCTGAAAGAAGAATCTCAGG | | |
| | | | AACTGGTTCAGTTGTACTCTTTAAG | | |
| | | | AACCCCTTTCTCTCTCCTGTTTGCC | | |
| | | | ATCCATTAAGAAAGCCATAT | | |
| | | | GATGCCTTTGGAGAAGGCAGACAC | | |
| | | | ACATTCCATTCCCAGCCTGCTCTGT | | |
| | | | GGGTAGGAGAATTTTCTACAG | | |
| | | | TAGGCAAATATGTGCTAAAGCCAA | | |
| | | | AGAGTTTTATAAGGAAATATATGT | | |
| | | | GCTCATGCAGTCAATACAGTTC | | |
| | | | TCAATCCCACCCAAAGCAGGTATG | | |
| | | | TCAATAAATCACATATTCCTAGGT | | |
| | | | GATACCCAAATGCTACAGAGTG | | |
| | | | CAACACTCAGACCTGAGATTTGCA | | |
| | | | AAAAGCAGATGTAAATATATGCAT | | |
| | | | TCAAACATCAGGGCTTACTATG | | |
| | | | AGGTAGGTGGTATATACATGTCAC | | |
| | | | AAATAAAAATACAGTTACAACTCA | | |
| | | | GGGTCACAAAAAATGCATCTTC | | |
| | | | CAATGCATATTTTTATTATGGTAAA | | |
| | | | ATATACATAAATATAATTCACCAT | | |
| | | | TTTAACATTTAATTCATATTA | | |
| | | | AATACGTACAAATCAGTGACATTT | | |
| | | | AGTACATTCACAGTGTTGTGCCAC | | |
| | | | CATCACCACTATTTAGTTCCAG | | |
| | | | AACATTTGCATCATCAATACATTGT | | |
| | | | CTAGAGACAAGACTATCCTGGGTA | | |
| | | | GGCAGAAACCATAGATCTTTT | | |
| | | | GTGTTTACAGCTATGGAAACCAAC | | |
| | | | TGTACCATAAAGATAGTTCACTGA | | |
| | | | GTTTTAAAGCCAAGCCACATCT | | |
| | | | TATTTTTCCAAGGTTTAATTTAGTG | | |
| | | | AGAGGGCAGCATTAGTGTGGAGTG | | |
| | | | GCATGCTTTTGCCCTATCGTG | | |
| | | | GAATTTACACATCAGAATGTGCAG | | |
| | | | GATCCAAGTCTGAAAGTGTTGCCA | | |
| | | | CCCGTCACACAACATGGGCTTT | | |
| | | | GTTTGCTTATTCCATGAAGCAGCA | | |
| | | | GCTATAGACCTTACCATGGAAACA | | |
| | | | TGAAGAGACCCTGCACCCCTTT | | |
| | | | CCTTAAGGATTGCTGCAAGAGTTA | | |
| | | | CCTGTTGAGCAGGATTGACTGGTG | | |
| | | | ATGTTTCATTCTGACCTTGTCC | | |
| | | | CAAGCTCTCCATCTCTAGATCTGG | | |
| | | | GGACTGACTGTTGAGCTGATGGGG | | |
| | | | AAAGAAAAGCTCTCACACAAAC | | |
| | | | CGGAAGCCAAATGTCCCCTATCTC | | |
| | | | TTGAATGATCAAGTCACTTTTGAC | | |
| | | | AACATCCAGGTGAATATAAAAA | | |
| | | | CTTAATAAAGCTGTGGAAAGGAAC | | |
| | | | TCTTAATCTTCTTTTCTGCTACTTA | | |
| | | | GGTTAAATTCACTAGATCTTG | | |
| | | | ATTAGGAATCAAAATTCGAATTGG | | |
| | | | GACATGTTCAAATTCTTTCTTGTGG | | |
| | | | TAGTTGCCTATACTGTCATCG | | |
| | | | CTGCTGTTGGTTGAGCATTTGTGGT | | |
| | | | GTACCACGCTGTGTGCTCAAGGGT | | |
| | | | ATTACATTCATCTTCTCATTT | | |
| | | | AATCCTCACAACAATCTGAAGAAG | | |
| | | | GTAGGTATTACAATTCCCACTTCAT | | |
| | | | AGAAACAGAAACTGAGGTTCA | | |
| | | | GAGAGGTTAAGTCATTTGCCCAAA | | |
| | | | TGGCTGAGCCAAAGCCTACCATGT | | |
| | | | ACCTAACCTTTATTTTCTTTCC | | |
| | | | CGAACATACCAGGCTGTCTCCTCA | | |
| | | | TAACTTCCAAGCATGCACTTAAAA | | |
| | | | CTCCACATGAATACAAGGTTCA | | |
| | | | TGGGACTTGGTATTCATAGAAAGG | | |
| | | | GAGGCAGAAAGCTGGTCTGTTCCT | | |
| | | | GATAGGCTTGTAATTTAATATC | | |
| | | | ATTCTGTTCATGTGCTTTGGATGGA | | |
| | | | AGCACATCTGGCATATGATGCTAA | | |
| | | | TCAGTGGTTCCCATACCCCTG | | |
| | | | GCTTCCTAATTTTAATGTTTGCTCA | | |
| | | | CAGCATAGTAGATTGACATCAAAT | | |
| | | | AGTGGCCGATGATGATGAAAA | | |
| | | | TAAACGTCAAATAAGTTGAGCCAA | | |
| | | | TAACAGCCGCTTTTTTCCTTCTGTC | | |
| | | | TGCGTATACAAAGCACTGTCA | | |
| | | | TGCACACAATCTATTCTGACCCTC | | |
| | | | ACAACAACCCATAAGGGTCTAAAT | | |
| | | | AGTATTTCCATTTTACAAATGA | | |
| | | | GGATCACACAAACTACTACATGGC | | |
| | | | AGAGCAGATACTCCAACTCATGTC | | |
| | | | TTCTGGTTGAAGCCTATTGCTT | | |
| | | | TTTCTTTTCTAAACACTTTCCCTCA | | |
| | | | GCAAGTTGGAATTAGACTTCACAA | | |
| | | | GTCTCCTTCAGACAACACAAA | | |
| | | | TCTTTTCTTATTCCATTCCTGTTTGG | | |
| | | | TTGCCTACGTCCAATCTCCCCCTCC | | |
| | | | CCAGAGATGCCAAAAAAAA | | |
| | | | AATCCTTTAAGGTATTTGGGAGCC | | |
| | | | AAACTCAACTTGTTAAAATCTCAA | | |
| | | | ATTATGGACACAATCACCAGAC | | |
| | | | ACAACCTAACCCCAATTATTTTGG | | |
| | | | CAGGAAGGTTGGTTTAGAGGCAGA | | |
| | | | TCCAGCAATCTGCTTTGGGCCA | | |
| | | | CTCTGGGTGGGGTAGGTGAAATAA | | |
| | | | GATTGGTCACTGTTAACTAATTTTA | | |
| | | | ATATTGGATTGGCCATTGGTT | | |
| | | | ATCACTGATTACCATTCTCCCCTGG | | |
| | | | ATTTTCACCCAGGACTCAAAACTT | | |
| | | | GGTTCTGCTAACCCTGTTCCT | | |
| | | | TTATGAGGAACCTTTTAAAGATTC | | |
| | | | CTTTATAAGGTGGGAGTTTTTTTTC | | |
| | | | TATGAACCTATAGGGGAGAAA | | |
| | | | AAAGATCAGCAGAAGTCATTACTT | | |
| | | | TTTTTTTTTTTTTTTTTTTTTTTTGA | | |
| | | | GAGAGAGTCTCACTCCATTG | | |
| | | | CCCAGGCTGGAGTGCAGTGGTGCT | | |
| | | | ATCTCGGCTCACTGCAACCTCCGC | | |
| | | | CTCCTGGGTTCAAGCAATTCTC | | |
| | | | CTGCCTCACCCTCCCGAGTAGCTG | | |
| | | | GGATTGCAGGTGCCCACCACCACA | | |
| | | | CCCGGCTAATTTTTGTATTTTT | | |
| | | | AGTAAAGACAGGGTTTCACCATGT | | |
| | | | TGGCCAGGCTGCTCTCCAACTCCC | | |
| | | | AATCTCAGGTGATCCTATTGCC | | |
| | | | TCGGGCTCCCAAAGTGCTGGGATT | | |
| | | | ACAGGAGTGAGCCACCATGCCTGG | | |
| | | | CCAGAAGTGGTTACTTCTGTAG | | |
| | | | ACAAAAGAATAATGCTACTTAATC | | |
| | | | AGGCTTTCTGTGTGACAAGAAAGA | | |
| | | | GAAAGAAAATAAAGAAGTTTCA | | |
| | | | ATTCATCCAATTCTTAATAAGAAA | | |
| | | | TATGTAAATAAAATTTTTTAAAATT | | |
| | | | ACACTTCATTTTAATGTTGTA | | |
| | | | TCAGTCAAGGTCCCTGCAAGAGAT | | |
| | | | GGATGGTATGGTACACTCAAACTG | | |
| | | | GGTAACACAGGAGAGTTTTCAG | | |
| | | | AAAGCAACTAAATCCAAAATACTA | | |
| | | | TCAAGGAATCAATATAAAAATTGT | | |
| | | | TAATATTTTTCTCATACTAAAT | | |
| | | | TTTCAAAATATTTTGTGTCTATTAC | | |
| | | | ATTTACAGCACATCTTAATTAGGA | | |
| | | | CTAGCTGTGTGTTCACCTCAC | | |
| | | | ATGTGGCTTGTAGCTACCATACTG | | |
| | | | GACAGCACATGTCCAAAAAAATAC | | |
| | | | ACGTAAAGTTAAAGTTTAAAAG | | |
| | | | ACACAGGAACTAAGCCCTCATTGT | | |
| | | | CTTTCCCTTGGGAGGTAGTTTAAA | | |
| | | | GAGCTATAGATGCTGTAACATT | | |
| | | | CTTGCTATTATTTATTATATATGAC | | |
| | | | ATTATTCCTAAAAAAGCTTTTGAG | | |
| | | | ATCCTAGGTTGTATTCCTCAG | | |
| | | | GTTTTGTTGCCTTCCCATGAAGATG | | |
| | | | TGAAGGCAGGGATGCCTGTTATTC | | |
| | | | AGTCCAAGATGCATGACAAGA | | |
| | | | GACCTTGGGAAAGTTTCATCTGGA | | |
| | | | TTTAAAGATTAATTCTTGATGCTTA | | |
| | | | CATTCCATACTCAAAATGTAA | | |
| | | | ATTTGAATATTAAAATAAAGATGA | | |
| | | | TTTTTTTTTTGGAGCTAGTCTTGCT | | |
| | | | CTGTTGCCCAGGCTGGAATGC | | |
| | | | AGTGGCATGATCATGGCTCACTGC | | |
| | | | AGCCTCGACCTCCCAAGCTCAAGC | | |
| | | | AAGGCTACAGGTGTGCACCTAA | | |
| | | | GTAGCTAGGACTACAGGTGTGCAC | | |
| | | | CACCATGTCTAGCTATTTTTTTTTC | | |
| | | | TGTAGAGACAGGGTTTTCCTA | | |
| | | | TGTTGTCCAGGCTGGTCTCGAACTC | | |
| | | | CTGCCCTCAAGCAATCCTCCTGCCT | | |
| | | | TGGCCTCCCAAAGTGTTGAG | | |
| | | | ATTACAGGCGTAAGCCACTGCACC | | |
| | | | TGGCCAAGATGAATATTTTAATAG | | |
| | | | CTCACAGAACAAAGTTTGCCAC | | |
| | | | ATAATGATAAAATTACTATGAAAA | | |
| | | | TATATTCCCTTTATTGTCAGTTTAA | | |
| | | | AAGATGAACTGAGTTTCACCC | | |
| | | | AAACTGGTCTGGCCCCTCTCTGATT | | |
| | | | CAAATACCAATAGTTGCTCTGATT | | |
| | | | CAAATTCCAACTGTTAGAACA | | |
| | | | TGACAGCTGCTCATAACTAGCTTT | | |
| | | | GCTTACTAACCATGTTTCTTTCCAT | | |
| | | | TTGTATTAGGTCCTTTACTTT | | |
| | | | TTATAACAGCCTCAAAGTTTCATG | | |
| | | | AATTGCTGCAGTAAACATTGATTTT | | |
| | | | CATGTTTGTGAGTCTGCAAGC | | |
| | | | CAGCTGGGCAGCTCTACTTCAGGT | | |
| | | | GGTAAGGGTGGATCAGACCTATTC | | |
| | | | CATATACCTCTTGTTCTCCTTG | | |
| | | | TCCAGTGGTTTCTAGGGATATGTTC | | |
| | | | TCATGATGAACCCCGCAGAGGCTC | | |
| | | | GTGAAAGTGAGAGGAAACTAG | | |
| | | | GATGCCTCTTAAGGTCTTGGTCAG | | |
| | | | GATGGGGTCTCCTGTCACTTCTGTC | | |
| | | | ACAGGCTATTGTAAGTCATAT | | |
| | | | GAGCAACCTCAATAAAATATAAAC | | |
| | | | AAGTCAGATAAACAGTGGGAGGA | | |
| | | | ATGGCAAAGTCATATGGCCAAGG | | |
| | | | CCATGAGTGATTAATTTTAACACA | | |
| | | | GGAAAAAACTAAAGCATTAAATGC | | |
| | | | GATTATTTAATATACAATGTCT | | |
| | | | TATTAACTGAAATATAAAATGTGT | | |
| | | | TTACTGTAAAATATAATCTGTTTAT | | |
| | | | CTCACCAAAGAAATATTATCT | | |
| | | | TTAAAAAATGTCATTACTTCTAAG | | |
| | | | ACATCATCAGTCTGCAACTTCTTTC | | |
| | | | CATAGCCTTAATCAGGATGCT | | |
| | | | GTGGCAGCTCCCACATTAGCCTCG | | |
| | | | CATTCTAAACTGGTAGATGTCCTA | | |
| | | | GGAAACCATACATCTATGTATT | | |
| | | | TTTCTTATTTTATACGTTTAGGACA | | |
| | | | ATGTATAGCTAATTACCCAACTTTT | | |
| | | | TATTTGCATACAAATCTAAT | | |
| | | | ACAACTGAACACAATCAGTTTTAT | | |
| | | | CACAGGTATAATGGATTTTTCAAT | | |
| | | | AGTGAGGAGGTGCCTCCATGAG | | |
| | | | CCTTCTCTTTAGAAAAGTGGCATTC | | |
| | | | AAGACTCTTCATTTGAAGTGAACA | | |
| | | | TTGCTATGTCTTTTGCATTGC | | |
| | | | TCTATTTTACATAAATTAAGTTATA | | |
| | | | AATTGACACTATAATCAACTGACA | | |
| | | | CCATCATCAGTGATCATGATC | | |
| | | | ACCCTCATCAGCACTAGAGTTGAC | | |
| | | | TTGTTTTTATAACCCCTTTGCATGT | | |
| | | | ATGTTGAATAGCAAAGTTCAT | | |
| | | | CAGAGAACATGTATTAGTCAATGG | | |
| | | | TAAGTAAGATACTCTCATCTAAGA | | |
| | | | AATAACATCACCTCTTCTAATG | | |
| | | | AAGTTCTAAGAAGAGAGGGAAGA | | |
| | | | AAAAGTCTTGGGAGCTAGTCAGGG | | |
| | | | AATAGTGTGTATTTGCAATTACC | | |
| | | | TAAACTGAACTCTACCATTACTCCT | | |
| | | | AACCCAGTTCCTCCTCCTGTGTTTT | | |
| | | | ACATGATTAATGCCACCCCT | | |
| | | | GCCTCAATGAACCAAGATCAGCTC | | |
| | | | CATCACTGGGACCTCCCCATTCTG | | |
| | | | CCTGTGCAATATTTTTCTTTTT | | |
| | | | TATTTCTCCTTCTAATATTACTGTT | | |
| | | | ATTGCTCCAGTAAAGAGCTGTAAT | | |
| | | | ATATTTTACCTGGACTGATAC | | |
| | | | CAGGAATGGTGGTGTTGCTTCCAA | | |
| | | | TCTGTTGCTGCTAGATTAATCTTTG | | |
| | | | CAAAGCACAGGGTTAATTTCA | | |
| | | | TTGCTGCTCAACTAAAACCACTGG | | |
| | | | TGGCTTTCCATTGCCTACAAAATA | | |
| | | | AAGTCAACCTCCCCATCAGACA | | |
| | | | TTCAAGGCTTTCAATGATCCATGG | | |
| | | | CCGCCAGCTCTCTCCAGGCTCATA | | |
| | | | TCCCACTCCACTCCTCTGATGT | | |
| | | | TTCCTACACTACACTACACTATACT | | |
| | | | ACACTACAGCCAGGTAGAATGACT | | |
| | | | GTTCACCCAACACCACTCAGG | | |
| | | | TTGTCTTCTCAACTTGGAATACTCT | | |
| | | | TGCACCTTCAAAGCTCATTTCAAAT | | |
| | | | GCCCCTTCATTTGTGAAGCC | | |
| | | | TTCTCCAAATTTCCAAGTCAGAAT | | |
| | | | GTCTCTTCCTTGTGCTACCACAACC | | |
| | | | CTTTAACTGAGCCTCCATTAG | | |
| | | | TGCACTGAGACCATTCTGTTCAGT | | |
| | | | GTCTGGGTGAAGCTTCCTGGTGAA | | |
| | | | AAATATGTTACCTATTTCTTTC | | |
| | | | TGAAAAGTTGGATTCAGGGATATT | | |
| | | | ATCACGGACCTAAGGTAATAGTTC | | |
| | | | TAGCCAACCTCCCTGTCCACTG | | |
| | | | CCAGGCCGACTACAAACCCTTCTG | | |
| | | | TTGCTGGCGAGCTGGTCCGCACCA | | |
| | | | CTAGTTCTGCTTCACTCTATTT | | |
| | | | ATCTCTTGATGTAACCATCTTCTTT | | |
| | | | CTCCAGGTTTTAAGAACCAGCCCA | | |
| | | | ACTCCTGGTTCCCTGATGAAG | | |
| | | | CTTTTATTCCCCTAGCCACATGGAA | | |
| | | | CTTTTCCTTTTTGGAACATGCCTTT | | |
| | | | AGTTTCTGTGTAGTTTGCCA | | |
| | | | TGCAGCACTTCATTGTACACATTAT | | |
| | | | TAAAACACAATTTTAAGGATTAGA | | |
| | | | ATGAACCTTAAAAGATCATGC | | |
| | | | ATCTCAAAATTTAATGTACATACA | | |
| | | | AATTACCCAGGGATTTTGTTGAAA | | |
| | | | TAAAAATTATTTAATTTTAATT | | |
| | | | AATATAAATAATTCAGTAGGTCTG | | |
| | | | GGGTGAGGCCTGAGGTTTTACATT | | |
| | | | TCCAACAAGCTGCCAGCTAAAG | | |
| | | | CCAATACATCTGTCCAGGAATCAC | | |
| | | | ACTTTGCGTATCAAAGGTCTACAT | | |
| | | | GACATTATCATTCCAAAGAGTT | | |
| | | | TCTTTTACAGGCTCTCAGATCAGTG | | |
| | | | TTCATCCACTACCTGACTACTGTCA | | |
| | | | TTCACAGGGATTCTGTTCCA | | |
| | | | CAGCAGGCCAGCTAACGTGGTATT | | |
| | | | TACAAAGCTCACTCCTCTTATACA | | |
| | | | ACAATCCAAGTGTTTCTTTTCT | | |
| | | | CAGTTGTCTGTGCCCCAGGAGATC | | |
| | | | CCTCTCTGCCTTGCCTTGCCCTCTG | | |
| | | | CCTTTGGAGACCAGCACCTCA | | |
| | | | TACTCAGTGAAGGCCTGGAGTGCT | | |
| | | | TAAGAGGGATTTCTTCCACCTCTCT | | |
| | | | TGCCCTGGTCTTCACTGTATT | | |
| | | | AGATGTATTACCTCCATGCTCTCAG | | |
| | | | TAGAGGCCCATAGGAAAGAGTAG | | |
| | | | GTAGGTTATGCCAGCTCACACG | | |
| | | | CATCCTTTAAAAATGGTTTAGAAG | | |
| | | | TTTAGCTGGTTTCTTATTACTCCTG | | |
| | | | TCTATGGATGTTTCCTTCTGT | | |
| | | | CACTCTACTAGGGATGAAACAGCT | | |
| | | | AATCATGTTCAATAGTTACATTTAG | | |
| | | | ATTGGTTTTTAAAAACTATGA | | |
| | | | TTGTATTAGTTCGTTTCCATGCTGC | | |
| | | | TGATAAAGACATATCTGAGACTGG | | |
| | | | AAACAAAAAGGGTTTAATTGG | | |
| | | | ACTTACAGTTCCACATGGCTGGGG | | |
| | | | AGGCCTCAAAATCAGGTGGGAGGC | | |
| | | | AAAAGGTACTTCTTACGTGGTG | | |
| | | | GCATCAAGAGCAAAATGAGGAAG | | |
| | | | AAGCAAAAGCAGAAACTCTTCATA | | |
| | | | AACCCACCAGATCTTGTGGGACT | | |
| | | | TATTATCACGAGAATACCACAGAA | | |
| | | | AAGACTGGCCTCCATGATTCAATT | | |
| | | | ACCTCCCACTGCGTCCCTCCCA | | |
| | | | CAACATGTGGGAATTCTGGGAGAT | | |
| | | | ACAATTCAAGTTGAGATTTGGGTG | | |
| | | | GGGACACAGCCAAACCATATCA | | |
| | | | TTCCTCCCTGGGCTCCTCCAAATTT | | |
| | | | CATAATCCTCACATTTCAAAACCA | | |
| | | | ATCATTCCTTCCCAACAGTTC | | |
| | | | CCCAAAGTCTTAACTCATTTCAGC | | |
| | | | ATTAACCCAAAAGTCCACAGTCCA | | |
| | | | AAGTCTCATCTGAGACAAGGCA | | |
| | | | AGTCCCTTCCACTTACAAGCCTGT | | |
| | | | AAAAGCAAGCTACTTACCTCCTAG | | |
| | | | ATACAATGGGGGGTACAGGTAT | | |
| | | | TGGGTAAATACAGCTGTTCCAAAT | | |
| | | | GAGAGAAATTGGCCAAAACAAAG | | |
| | | | GGGTTACAGGGTCCATGCAAGTC | | |
| | | | TGAAATCCAGTGGGGCAGTCAAAT | | |
| | | | TTTAAAGCTCCATAATGATCTCCTT | | |
| | | | TGACTCCATGTCTCACATTCA | | |
| | | | GGTCATGCTGATGCAAGAGATAGG | | |
| | | | TTCCCATGGTCTTGTCCAGCTCCGC | | |
| | | | CCCTGTGGCTTTGCAGAGTAC | | |
| | | | AGCCTCCCTCCTGGCTGCTTTCTCA | | |
| | | | GGCTGATGTTGAGTGTCTGTAGCTT | | |
| | | | TTCCAGGCACAAGATGCAAG | | |
| | | | TTGGTGGTTGATCTACCATTCTGGG | | |
| | | | GTCTACCATTCTGGGGTCTACCGTT | | |
| | | | CTGGGACTGTGGCCTTCTTC | | |
| | | | TCACAGCTCCACTAGGCAGTGCCC | | |
| | | | CAACAGGGACTCTGTGTGGGGGCT | | |
| | | | CTGCCCCACATTTCCCTTCCAC | | |
| | | | ACTGCCCTAGGAGAGGTTCCCCAT | | |
| | | | GAGGGCTCTGCCCCTGCAGCAAAC | | |
| | | | TTTTGCCTGGACATCCAGGTGT | | |
| | | | TTCCATATATATTCTGAAATCTAGG | | |
| | | | CAGAGGTTCCCAAATCTCAATTCTT | | |
| | | | GACATCTCTGCACCCACAGG | | |
| | | | CTCAACATCACATGGAAGCTGCCA | | |
| | | | ATGCTTGGGGCCTCTACCCTCTGA | | |
| | | | AGCCACAGCCCAAGCTCTATGT | | |
| | | | TGGCTCCTTTCAGCCATGGCTGGA | | |
| | | | GCAGCTGGGACACAGGGCACCAA | | |
| | | | GTCCCTAGGCTGCACACAGCACA | | |
| | | | GAGACCCTGGGCCCAGCCCACAAA | | |
| | | | ACCACTTTTTCCTCCTGGGCCTCTG | | |
| | | | GGCCTGTGATGGGAGGGGCTG | | |
| | | | CCATGAAGGTCTCTGACATGACCT | | |
| | | | GGAGACATTTTCCCCATGGTCTTG | | |
| | | | GGGATTAACATTAGGCTCCTTG | | |
| | | | CTGCTTATGCAAATTTCTGCAGCCA | | |
| | | | GCTTGAATTTCTCCTTAAAAAAAA | | |
| | | | TGGGTTTTTCTTTTCTACTGC | | |
| | | | ATCATCAGGCTGCAGATTTTCCAC | | |
| | | | ATTTATGCTCTTGTTTCCCTTTTAA | | |
| | | | AACAGAATGTTTTTAACAGCA | | |
| | | | CCCAAGTCACCTTTTGAATGCTTTG | | |
| | | | CTGCTTAGAAATTTATTCCACCAG | | |
| | | | ATACCCTAAGTCATCTCTCTC | | |
| | | | AAGCTCTAAGTTCCACAAATCTCT | | |
| | | | AGGGCAAGGGTGAAATGCTGCCAG | | |
| | | | TCTCCTTGCTAAAACATAACAA | | |
| | | | GGGTCACCTTTACTTCAGTTCCCAA | | |
| | | | CAAGGTCTTCATCTCCATCTGAGA | | |
| | | | CCACCTCAGCCTGGACCTTAT | | |
| | | | TGTTCATATCACTATCAGTATTTTT | | |
| | | | GTCAATGCCATTCACAGTCTCTAG | | |
| | | | GAGGTTCCAAACTTTCCTACA | | |
| | | | TTTTCCTATCTTCTTCTGAGCCCTC | | |
| | | | CAGATTATTTCAACACCCAGTTCC | | |
| | | | AAAGTTGCTTCCACATTTTCG | | |
| | | | GGTATCTTTTCAGCAATGCCCCACT | | |
| | | | CTACTGGTACTATTAGTCCATTTTC | | |
| | | | ATGCTGCTGATAAAGACATA | | |
| | | | CCTGAGACTGGGAACAAAAAGAG | | |
| | | | GTTTAATTGGACTTATAGTTCCACC | | |
| | | | TGGCTGGGGAGGCCTCAGAATC | | |
| | | | ATGGCAGGAGGTGAAAGGCATTTC | | |
| | | | TTACACGGCAGCAGCAAGAGAAA | | |
| | | | AATGAAGAAGCAGCAAAAGCAGA | | |
| | | | AACCCCTGATAAAACCATCAGATC | | |
| | | | TCGTGAGACTTATTCACTATCACA | | |
| | | | AGAATAGCATGGGAAAGACCAG | | |
| | | | CCCCCTTGATTCAATTACCTCCCCC | | |
| | | | TGGGTCCTGTGGGAATTCTGGAAG | | |
| | | | GTACAATTCAAGTTGAGATTT | | |
| | | | GGGTGGGGACACAGCCAAACCAT | | |
| | | | ATCAATGATTTTGTACTTTAACCAG | | |
| | | | CTGAATGGAAGTACAATCTCTT | | |
| | | | GCTATATGACACAATAATTATTTG | | |
| | | | CAAAATGAGTAAACATATCATAAG | | |
| | | | GAAATTATTTTTACAAGGTTTG | | |
| | | | AAACCTGAAATGCAGTCTATTATC | | |
| | | | ATACATAACTAAAAATAGAGCCTC | | |
| | | | AATAAACAGATTCCCAGTTTTG | | |
| | | | AAAATGCAACATTTGTACTCCACA | | |
| | | | TTGTCAGTTTTCTTAGGTATATTTA | | |
| | | | TAAATACTCCTATAAAAATGT | | |
| | | | AAAGAAACACATAATGTAGATTGC | | |
| | | | TAATTTTATAATAACACAAGTTGA | | |
| | | | TTTTGACATCCAACTTATTAAT | | |
| | | | TATGAAATGACTTTTGGCCTAGTA | | |
| | | | ACAATGAAAATGGGGGCAAATAC | | |
| | | | AGATAAATGGTAATTCTTAGAAT | | |
| | | | GAACTACTCAGCACCAATTCTAAG | | |
| | | | TTTTTCTTGATGGTAAATCATAATG | | |
| | | | TTCCCTTTCTCCTCGGTTCTG | | |
| | | | CAATCTATAGGCATACCATAATTG | | |
| | | | TAATCAATAGCTTAAAAATATGTC | | |
| | | | TCTCTGTCCTATTCTGTATCTG | | |
| | | | TATCTCTTGGATTTTTACCTTTGCA | | |
| | | | ATAGTCAACTGAACCATCTTCTTG | | |
| | | | GAGTACTCATGAAGATGGAAG | | |
| | | | TCTACATGGAGAATACAGGATGAA | | |
| | | | TCCACTGTGTCTCCTGCAGTGAAGT | | |
| | | | CTGTTTGAAGGATGTATTTGG | | |
| | | | CTGTCTTCTGGACAGGCCATTCTAA | | |
| | | | TAACAGAAACAAACAAGTTATTTT | | |
| | | | AAAACTTATTGGAATATTCAA | | |
| | | | ATATTAACCAAAGTAGAAAAATAT | | |
| | | | AATACACATCCATGTGCCCATCAC | | |
| | | | AGAACTTCACTGATTATCATCA | | |
| | | | TTTAGCCAGTCTTGAAGAAGCAAG | | |
| | | | TGCTAATTACAATCACAAATGAAA | | |
| | | | CAAGATTCAGACTTCATGAAGA | | |
| | | | GCACTGCGCTATAATAAAAGAAGA | | |
| | | | AATGAGCACATACATTCTTTTACTG | | |
| | | | ACAGTCAAATGGTGAAGGTGG | | |
| | | | GCAGAATCATTATGTGATGCAACA | | |
| | | | TGGCAAAAGTATACAGACAGTGCA | | |
| | | | TCCAGAGGAAGGCACCTTGCTG | | |
| | | | AATGACTAGAATGGAAGTAGGAG | | |
| | | | ACATTTTGCAGGCCCCCTTCATCCT | | |
| | | | GCAGGGAGAACCAGAACCACAG | | |
| | | | CAGCTCTATTTGCCTATTCCTCTTT | | |
| | | | AAATTACAAAGTTAAAATTTGGGA | | |
| | | | GTAGTAGAAAATCAATTGGTT | | |
| | | | ATCTTATAGAGTCTCCTAGAATATT | | |
| | | | TCATTGGCATTGAGAAGGTGGAAA | | |
| | | | ATGCAAATTATATACTTTAAA | | |
| | | | ATGTAATTTTTGCTTTTCACATATG | | |
| | | | CTTAAACCCTAAAACCTCTTAATA | | |
| | | | AACTTCTTCTGAAATATA (SEQ ID | | |
| | | | NO: 41) | | |
| | | NM_001164182.2 | ACCGAGACCGACCACAGCAAGCA | NP_001157654.1 | MESEELADRVLDVVER |
| | | | GAGGCTGGGGGGGGGAAAGACGA | | SLSNYPFDFQGARHTGQ |
| | | | GGAAAGAGGAGGAAAACAAAAGC | | EEGAYGWITI |
| | | | T | | NYLLGKFSQKTRWFSIV |
| | | | GCTACTTATGGAAGATACAAAGGA | | PYETNNQETFGALDLGG |
| | | | GTCTAACGTGAAGACATTTTGCTC | | ASTQVTFVPQNQTIESP |
| | | | CAAGAATATCCTAGCCATCCTT | | DNALQFR |
| | | | GGCTTCTCCTCTATCATAGCTGTGA | | LYGKDYNVYTHSFLCY |
| | | | TAGCTTTGCTTGCTGTGGGGTTGAC | | GKDQALWQKLAKDIQV |
| | | | CCAGAACAAAGCATTGCCAG | | ASNEILRDPCFHPGYKK |
| | | | AAAACGTTAACTATGGGATTGTCC | | VVNVSDLYK |
| | | | TGGATGCGGGTTCTTCTCACACAA | | TPCTKRFEMTLPFQQFEI |
| | | | GTTTATACATCTATAAGTGGCC | | QGIGNYQQCHQSILELF |
| | | | AGCAGAAAAGGAGAATGACACAG | | NTSYCPYSQCAFNGIFL |
| | | | GCGTGGTGCATCAAGTAGAAGAAT | | PPLQGD |
| | | | GCAGGGTTAAAGGATGGAAAGTG | | FGAFSAFYFVMKFLNLT |
| | | | AAGAGTTGGCAGACAGGGTTCTGG | | SEKVSQEKVTEMMKKF |
| | | | ATGTGGTGGAGAGGAGCCTCAGCA | | CAQPWEEIKTSYAGVKE |
| | | | ACTACCCCTTTGACTTCCAGGG | | KYLSEYCF |
| | | | TGCCAGGATCATTACTGGCCAAGA | | SGTYILSLLLQGYHFTA |
| | | | GGAAGGTGCCTATGGCTGGATTAC | | DSWEHIHFIGKIQGSDA |
| | | | TATCAACTATCTGCTGGGCAAA | | GWTLGYMLNLTNMIPA |
| | | | TTCAGTCAGAAAACAAGGTGGTTC | | EQPLSTPL |
| | | | AGCATAGTCCCATATGAAACCAAT | | SHSTYVFLMVLFSLVLF |
| | | | AATCAGGAAACCTTTGGAGCTT | | TVAIIGLLIFHKPSYFWK |
| | | | TGGACCTTGGGGGAGCCTCTACAC | | DMV (SEQ ID NO: 44) |
| | | | AAGTCACTTTTGTACCCCAAAACC | | |
| | | | AGACTATCGAGTCCCCAGATAA | | |
| | | | TGCTCTGCAATTTCGCCTCTATGGC | | |
| | | | AAGGACTACAATGTCTACACACAT | | |
| | | | AGCTTCTTGTGCTATGGGAAG | | |
| | | | GATCAGGCACTCTGGCAGAAACTG | | |
| | | | GCCAAGGACATTCAGGTTGCAACT | | |
| | | | AATGAAATTCTCAGGGACCCAT | | |
| | | | GCTTTCATCCTGGATATAAGAAGG | | |
| | | | TAGTGAACGTAAGTGACCTTTACA | | |
| | | | AGACCCCCTGCACCAAGAGATT | | |
| | | | TGAGATGACTCTTCCATTCCAGCA | | |
| | | | GTTTGAAATCCAGGGTATTGGAAA | | |
| | | | CTATCAACAATGCCATCAAAGC | | |
| | | | ATCCTGGAGCTCTTCAACACCAGT | | |
| | | | TACTGCCCTTACTCCCAGTGTGCCT | | |
| | | | TCAATGGGATTTTCTTGCCAC | | |
| | | | CACTCCAGGGGGATTTTGGGGCAT | | |
| | | | TTTCAGCTTTTTACTTTGTGATGAA | | |
| | | | GTTTTTAAACTTGACATCAGA | | |
| | | | GAAAGTCTCTCAGGAAAAGGTGAC | | |
| | | | TGAGATGATGAAAAAGTTCTGTCC | | |
| | | | TCAGCCTTGGGAGGAGATAAAA | | |
| | | | ACATCTTACGCTGGAGTAAAGGAG | | |
| | | | AAGTACCTGAGTGAATACTGCTTT | | |
| | | | TCTGGTACCTACATTCTCTCCC | | |
| | | | TCCTTCTGCAAGGCTATCATTTCAC | | |
| | | | AGCTGATTCCTGGGAGCACATCCA | | |
| | | | TTTCATTGGCAAGATCCAGGG | | |
| | | | CAGCGACGCCGGCTGGACTTTGGG | | |
| | | | CTACATGCTGAACCTGACCAACAT | | |
| | | | GATCCCAGCTGACCAACCATTG | | |
| | | | TCCACACCTCTCTCCCACTCCACCT | | |
| | | | ATGTCTTCCTCATGGTTCTATTCTC | | |
| | | | CCTGGTCCTTTTCACAGTGG | | |
| | | | CCATCATAGGCTTGCTTATCTTTCA | | |
| | | | CAAGCCTTCATATTTCTGGAAAGA | | |
| | | | TATGGTATAGCAAAAGCAGCT | | |
| | | | GAAATATGCTGGCTGGAGTGAGGA | | |
| | | | AAAAAATCGTCCAGGGAGCATTTT | | |
| | | | CCTCCATCGCAGTGTTCAAGGC | | |
| | | | CATCCTTCCCTGTCTGCCAGGGCC | | |
| | | | AGTCTTGACGAGTGTGAAGCTTCC | | |
| | | | TTGGCTTTTACTGAAGCCTTTC | | |
| | | | TTTTGGAGGTATTCAATATCCTTTG | | |
| | | | CCTCAAGGACTTCGGCAGATACTG | | |
| | | | TCTCTTTCATGAGTTTTTCCC | | |
| | | | AGCTACACCTTTCTCCTTTGTACTT | | |
| | | | TGTGCTTGTATAGGTTTTAAAGACC | | |
| | | | TGACACCTTTCATAATCTTT | | |
| | | | GCTTTATAAAAGAACAATATTGAC | | |
| | | | TTTGTCTAGAAGAACTGAGAGTCT | | |
| | | | TGAGTCCTGTGATAGGAGGCTG | | |
| | | | AGCTGGCTGAAAGAAGAATCTCAG | | |
| | | | GAACTGGTTCAGTTGTACTCTTTAA | | |
| | | | GAACCCCTTTCTCTCTCCTGT | | |
| | | | TTGCCATCCATTAAGAAAGCCATA | | |
| | | | TGATGCCTTTGGAGAAGGCAGACA | | |
| | | | CACATTCCATTCCCAGCCTGCT | | |
| | | | CTGTGGGTAGGAGAATTTTCTACA | | |
| | | | GTAGGCAAATATGTGCTAAAGCCA | | |
| | | | AAGAGTTTTATAAGGAAATATA | | |
| | | | TGTGCTCATGCAGTCAATACAGTT | | |
| | | | CTCAATCCCACCCAAAGCAGGTAT | | |
| | | | GTCAATAAATCACATATTCCTA | | |
| | | | GGTGATACCCAAATGCTACAGAGT | | |
| | | | GGAACACTCAGACCTGAGATTTGC | | |
| | | | AAAAAGCAGATGTAAATATATG | | |
| | | | CATTCAAACATCAGGGCTTACTAT | | |
| | | | GAGGTAGGTGGTATATACATGTCA | | |
| | | | CAAATAAAAATACAGTTACAAC | | |
| | | | TCAGGGTCACAAAAAATGCATCTT | | |
| | | | CCAATGCATATTTTTATTATGGTAA | | |
| | | | AATATACATAAATATAATTCA | | |
| | | | CCATTTTAACATTTAATTCATATTA | | |
| | | | AATACGTACAAATCAGTGACATTT | | |
| | | | AGTACATTCACAGTGTTGTGC | | |
| | | | CACCATCACCACTATTTAGTTCCA | | |
| | | | GAACATTTGCATCATCAATACATT | | |
| | | | GTCTAGAGACAAGACTATCCTG | | |
| | | | GGTAGGCAGAAACCATAGATCTTT | | |
| | | | TGTGTTTACAGCTATGGAAACCAA | | |
| | | | CTGTACCATAAAGATAGTTCAC | | |
| | | | TGAGTTTTAAAGCCAAGCCACATC | | |
| | | | TTATTTTTCCAAGGTTTAATTTAGT | | |
| | | | GAGAGGGCAGCATTAGTGTGG | | |
| | | | AGTGGCATGCTTTTGCCCTATCGTG | | |
| | | | GAATTTACACATCAGAATGTGCAG | | |
| | | | GATCCAAGTCTGAAAGTGTTG | | |
| | | | CCACCCGTCACACAACATGGGCTT | | |
| | | | TGTTTGCTTATTCCATGAAGCAGCA | | |
| | | | GCTATAGACCTTACCATGGAA | | |
| | | | ACATGAAGAGACCCTGCACCCCTT | | |
| | | | TCCTTAAGGATTGCTGCAAGAGTT | | |
| | | | ACCTGTTGAGCAGGATTGACTG | | |
| | | | GTGATGTTTCATTCTGACCTTGTCC | | |
| | | | CAAGCTCTCCATCTCTAGATCTGG | | |
| | | | GGACTGACTGTTGAGCTGATG | | |
| | | | GGGAAAGAAAAGCTCTCACACAA | | |
| | | | ACCGGAAGCCAAATGTCCCCTATC | | |
| | | | TCTTGAATGATCAAGTCACTTTT | | |
| | | | GACAACATCCAGGTGAATATAAAA | | |
| | | | ACTTAATAAAGCTGTGGAAAGGAA | | |
| | | | CTCTTAATCTTCTTTTCTGCTA | | |
| | | | CTTAGGTTAAATTCACTAGATCTTG | | |
| | | | ATTAGGAATCAAAATTCGAATTGG | | |
| | | | GACATGTTCAAATTCTTTCTT | | |
| | | | GTGGTAGTTGCCTATACTGTCATCG | | |
| | | | CTGCTGTTGGTTGAGCATTTGTGGT | | |
| | | | GTACCACGCTGTGTGCTCAA | | |
| | | | GGGTATTACATTCATCTTCTCATTT | | |
| | | | AATCCTCACAACAATCTGAAGAAG | | |
| | | | GTAGGTATTACAATTCCCACT | | |
| | | | TCATAGAAACAGAAACTGAGGTTC | | |
| | | | AGAGAGGTTAAGTCATTTGCCCAA | | |
| | | | ATGGCTGAGCCAAAGCCTACCA | | |
| | | | TGTACCTAACCTTTATTTTCTTTCC | | |
| | | | CGAACATACCAGGCTGTCTCCTCA | | |
| | | | TAACTTCCAAGCATGCACTTA | | |
| | | | AAACTCCACATGAATACAAGGTTC | | |
| | | | ATGGGACTTGGTATTCATAGAAAG | | |
| | | | GGAGGCAGAAAGCTGGTCTGTT | | |
| | | | CCTGATAGGCTTGTAATTTAATATC | | |
| | | | ATTCTGTTCATGTGCTTTGGATGGA | | |
| | | | AGCACATCTGGCATATGATG | | |
| | | | CTAATCAGTGGTTCCCATACCCCT | | |
| | | | GGCTTCCTAATTTTAATGTTTGCTC | | |
| | | | ACAGCATAGTAGATTGACATC | | |
| | | | AAATAGTGGCCGATGATGATGAAA | | |
| | | | ATAAAGGTCAAATAAGTTGAGCCA | | |
| | | | ATAACAGCCGCTTTTTTCCTTC | | |
| | | | TGTCTGCGTATACAAAGCACTGTC | | |
| | | | ATGCACACAATCTATTCTGACCCT | | |
| | | | CACAACAACCCATAAGGGTGTA | | |
| | | | AATACTATTTCCATTTTACAAATGA | | |
| | | | GGATCACACAAACTACTACATGGC | | |
| | | | AGAGCAGATACTCCAACTCAT | | |
| | | | GTCTTCTGGTTGAAGCCTATTGCTT | | |
| | | | TTTCTTTTCTAAACACTTTCCCTCA | | |
| | | | GCAAGTTGGAATTAGACTTC | | |
| | | | ACAAGTCTCCTTCAGAGAACACAA | | |
| | | | ATCTTTTCTTATTCCATTCCTGTTTG | | |
| | | | GTTGCCTACGTCCAATCTCC | | |
| | | | CCCTCCCCAGAGATGCCAAAAAAA | | |
| | | | AAATCCTTTAAGGTATTTGGGAGC | | |
| | | | CAAACTCAACTTGTTAAAATCT | | |
| | | | CAAATTATGGAGACAATCACCAGA | | |
| | | | CACAACCTAACCCCAATTATTTTG | | |
| | | | GCAGGAAGGTTGGTTTAGAGGC | | |
| | | | AGATCCAGCAATCTGCTTTGGGCC | | |
| | | | ACTCTGGGTGGGGTAGGTGAAATA | | |
| | | | AGATTGGTCACTGTTAACTAAT | | |
| | | | TTTAATATTGGATTGGCCATTGGTT | | |
| | | | ATCACTGATTACCATTCTCCCCTGG | | |
| | | | ATTTTCACCCAGCACTCAAA | | |
| | | | ACTTGGTTCTGCTAACCCTGTTCCT | | |
| | | | TTATGAGGAACCTTTTAAAGATTC | | |
| | | | CTTTATAAGGTGGGAGTTTTT | | |
| | | | TTTCTATGAACCTATAGGGGAGAA | | |
| | | | AAAAGATCAGCAGAAGTCATTACT | | |
| | | | TTTTTTTTTTTTTTTTTTTTTT | | |
| | | | TTTGAGAGAGAGTCTCACTCCATT | | |
| | | | GCCCAGGCTGGAGTGCAGTGGTGC | | |
| | | | TATCTCGGCTCACTGCAACCTC | | |
| | | | CGCCTCCTGGCTTCAACCAATTCT | | |
| | | | CCTGCCTCAGCCTCCCGAGTAGCT | | |
| | | | GGGATTGCAGGTGCCCACCACC | | |
| | | | ACACCCGGCTAATTTTTGTATTTTT | | |
| | | | AGTAAAGACAGGGTTTCACCATGT | | |
| | | | TGGCCAGGCTGGTCTCCAACT | | |
| | | | CCCAATCTCAGGTGATCCTATTGC | | |
| | | | CTCGGGCTCCCAAAGTGCTGGGAT | | |
| | | | TACAGGAGTGAGCCACCATGCC | | |
| | | | TGGCCAGAAGTGGTTACTTCTGTA | | |
| | | | GACAAAAGAATAATGCTACTTAAT | | |
| | | | CAGGCTTTCTGTGTGACAAGAA | | |
| | | | AGAGAAAGAAAATAAAGAAGTTT | | |
| | | | CAATTCATCCAATTCTTAATAAGA | | |
| | | | AATATGTAAATAAAATTTTTTAA | | |
| | | | AATTACACTTCATTTTAATGTTGTA | | |
| | | | TCAGTCAAGGTCCCTGCAAGAGAT | | |
| | | | GGATGGTATGGTACACTCAAA | | |
| | | | CTGGGTAACACAGGAGAGTTTTCA | | |
| | | | CAAAGCAACTAAATCCAAAATACT | | |
| | | | ATCAAGGAATCAATATAAAAAT | | |
| | | | TGTTAATATTTTTCTCATACTAAAT | | |
| | | | TTTCAAAATATTTTGTGTCTATTAC | | |
| | | | ATTTACAGCACATCTTAATT | | |
| | | | AGGACTACCTGTGTGTTCACCTCA | | |
| | | | CATGTGGCTTGTAGCTACCATACT | | |
| | | | GGACAGCACATGTCCAAAAAAA | | |
| | | | TACACGTAAAGTTAAAGTTTAAAA | | |
| | | | GACACAGGAACTAAGCCCTCATTG | | |
| | | | TCTTTCCCTTGGGAGGTAGTTT | | |
| | | | AAAGAGCTATAGATGCTGTAACAT | | |
| | | | TCTTGCTATTATTTATTATATATGA | | |
| | | | CATTATTCCTAAAAAAGCTTT | | |
| | | | TGAGATCCTAGGTTGTATTCCTCAG | | |
| | | | GTTTTGTTGCCTTCCCATGAAGATG | | |
| | | | TGAAGGCAGGGATGCCTGTT | | |
| | | | ATTCAGTCCAAGATGCATGACAAG | | |
| | | | AGACCTTGGGAAAGTTTCATCTGG | | |
| | | | ATTTAAAGATTAATTCTTGATG | | |
| | | | CTTACATTCCATACTCAAAATGTA | | |
| | | | AATTTGAATATTAAAATAAAGATG | | |
| | | | ATTTTTTTTTTGGAGCTAGTCT | | |
| | | | TGCTCTGTTGCCCAGGCTGGAATG | | |
| | | | CAGTGGCATGATCATGGCTCACTG | | |
| | | | CAGCCTCGACCTCCCAAGCTCA | | |
| | | | AGCAAGGCTACAGGTGTGCACCTA | | |
| | | | AGTACCTAGGACTACAGGTGTGCA | | |
| | | | CCACCATGTCTAGCTATTTTTT | | |
| | | | TTTCTGTAGAGACAGGGTTTTCCTA | | |
| | | | TGTTGTCCAGGCTGGTCTCGAACTC | | |
| | | | CTGCCCTCAACCAATCCTCC | | |
| | | | TGCCTTGGCCTCCCAAAGTGTTGA | | |
| | | | GATTACAGGCGTAAGCCACTGCAC | | |
| | | | CTGGCCAAGATGAATATTTTAA | | |
| | | | TAGCTCACAGAACAAAGTTTGCCA | | |
| | | | CATAATGATAAAATTACTATGAAA | | |
| | | | ATATATTCCCTTTATTGTCAGT | | |
| | | | TTAAAAGATGAACTGAGTTTCACC | | |
| | | | CAAACTGGTCTGGCCCCTCTCTGA | | |
| | | | TTCAAATACCAATAGTTGCTCT | | |
| | | | GATTCAAATTCCAACTGTTAGAAC | | |
| | | | ATGACAGCTGCTCATAACTAGCTT | | |
| | | | TGCTTACTAACCATGTTTCTTT | | |
| | | | CCATTTGTATTAGGTCCTTTACTTT | | |
| | | | TTATAACAGCCTCAAAGTTTCATG | | |
| | | | AATTGCTGCAGTAAACATTGA | | |
| | | | TTTTCATGTTTGTGAGTCTGCAAGC | | |
| | | | CAGCTGGGCAGCTCTACTTCAGGT | | |
| | | | GGTAAGGGTGGATCAGACCTA | | |
| | | | TTCCATATACCTCTTGTTCTCCTTG | | |
| | | | TCCAGTGGTTTCTAGGGATATGTTC | | |
| | | | TCATGATGAACCCCGCAGAG | | |
| | | | GCTCGTGAAAGTGAGAGGAAACTA | | |
| | | | GGATGCCTCTTAAGCTCTTGGTCA | | |
| | | | GGATGGGGTCTCCTGTCACTTC | | |
| | | | TGTCACAGGCTATTGTAAGTCATA | | |
| | | | TGAGCAAGCTCAATAAAATATAAA | | |
| | | | CAAGTCAGATAAACAGTGGGAG | | |
| | | | GAATGGCAAAGTCATATGGCCAAG | | |
| | | | GCCATGAGTGATTAATTTTAACAC | | |
| | | | AGGAAAAAAGTAAAGCATTAAA | | |
| | | | TGCGATTATTTAATATACAATGTCT | | |
| | | | TATTAACTGAAATATAAAATGTGT | | |
| | | | TTACTGTAAAATATAATCTGT | | |
| | | | TTATCTCACCAAAGAAATATTATCT | | |
| | | | TTAAAAAATGTCATTACTTCTAAG | | |
| | | | ACATCATCAGTCTGCAACTTC | | |
| | | | TTTCCATAGCCTTAATCAGGATGCT | | |
| | | | GTGGCAGCTCCCACATTAGCCTCG | | |
| | | | CATTCTAAACTGGTAGATGTC | | |
| | | | CTAGGAAACCATACATCTATGTAT | | |
| | | | TTTTCTTATTTTATACGTTTAGGAC | | |
| | | | AATGTATACCTAATTACCCAA | | |
| | | | CTTTTTATTTGCATACAAATCTAAT | | |
| | | | ACAACTGAACACAATCAGTTTTAT | | |
| | | | CACAGGTATAATGGATTTTTC | | |
| | | | AATAGTGAGGAGGTGCCTCCATGA | | |
| | | | GCCTTCTCTTTAGAAAAGTGGCATT | | |
| | | | CAAGACTCTTCATTTGAAGTG | | |
| | | | AAGATTGCTATGTCTTTTGCATTGC | | |
| | | | TCTATTTTACATAAATTAAGTTATA | | |
| | | | AATTGACACTATAATCAACT | | |
| | | | GACACCATGATCAGTGATGATGAT | | |
| | | | CACCCTCATCAGCACTAGAGTTGA | | |
| | | | CTTGTTTTTATAACCCCTTTGC | | |
| | | | ATGTATGTTGAATAGCAAAGTTCA | | |
| | | | TCAGAGAACATGTATTAGTCAATG | | |
| | | | GTAAGTAAGATACTCTCATCTA | | |
| | | | AGAAATAACATCACCTCTTCTAAT | | |
| | | | GAAGTTCTAAGAAGAGAGGGAAG | | |
| | | | AAAAAGTCTTGGGACCTAGTCAG | | |
| | | | GGAATAGTGTGTATTTGCAATTAC | | |
| | | | CTAAACTGAACTCTACCATTACTC | | |
| | | | CTAACCCAGTTCCTCCTCCTGT | | |
| | | | GTTTTACATGATTAATGCCACCCCT | | |
| | | | GCCTCAATGAACCAAGATCAGCTC | | |
| | | | CATCACTGGGACCTCCCCATT | | |
| | | | CTGCCTGTGCAATATTTTTCTTTTT | | |
| | | | TATTTCTCCTTCTAATATTACTGTT | | |
| | | | ATTGCTCCAGTAAAGAGCTG | | |
| | | | TAATATATTTTACCTGGACTGATAC | | |
| | | | CAGGAATGGTGGTGTTGCTTCCAA | | |
| | | | TCTGTTGCTGCTAGATTAATC | | |
| | | | TTTGCAAAGCACAGGCTTAATTTC | | |
| | | | ATTGCTGCTCAACTAAAACCACTG | | |
| | | | GTGGCTTTCCATTGCCTACAAA | | |
| | | | ATAAAGTCAACCTCCCCATCAGAC | | |
| | | | ATTCAAGGCTTTCAATGATCCATG | | |
| | | | GCCGCCAGCTCTCTCCAGGCTC | | |
| | | | ATATCCCACTCCACTCCTCTGATGT | | |
| | | | TTCCTACACTACACTACACTATACT | | |
| | | | ACACTACAGCCAGGTAGAAT | | |
| | | | GACTGTTCACCCAACACCACTCAG | | |
| | | | GTTGTCTTCTCAACTTGGAATACTC | | |
| | | | TTGCACCTTCAAAGCTCATTT | | |
| | | | CAAATGCCCCTTCATTTGTGAAGC | | |
| | | | CTTCTCCAAATTTCCAAGTCAGAAT | | |
| | | | GTCTCTTCCTTGTGCTACCAC | | |
| | | | AACCCTTTAACTGAGCCTCCATTA | | |
| | | | GTGCACTGAGACCATTCTGTTCAG | | |
| | | | TGTCTGGGTGAAGCTTCCTGGT | | |
| | | | GAAAAATATGTTACCTATTTCTTTC | | |
| | | | TGAAAAGTTGGATTCAGGGATATT | | |
| | | | ATCACGGACCTAAGGTAATAG | | |
| | | | TTCTAGCCAACCTCCCTGTCCACTG | | |
| | | | CCAGGCCGACTACAAACCCTTCTG | | |
| | | | TTGCTGGCGAGCTGGTCCGCA | | |
| | | | CCACTAGTTCTGCTTCACTCTATTT | | |
| | | | ATCTCTTGATGTAACCATCTTCTTT | | |
| | | | CTCCAGGTTTTAAGAACCAG | | |
| | | | CCCAACTCCTGGTTCCCTGATGAA | | |
| | | | GCTTTTATTCCCCTAGCCACATGGA | | |
| | | | ACTTTTCCTTTTTGGAACATG | | |
| | | | CCTTTAGTTTCTGTGTAGTTTGCCA | | |
| | | | TGCAGCACTTCATTGTACACATTAT | | |
| | | | TAAAACAGAATTTTAAGGAT | | |
| | | | TAGAATGAACCTTAAAAGATCATG | | |
| | | | CATCTCAAAATTTAATGTACATAC | | |
| | | | AAATTACCCAGGGATTTTGTTG | | |
| | | | AAATAAAAATTATTTAATTTTAATT | | |
| | | | AATATAAATAATTCAGTAGGTCTG | | |
| | | | GGGTGAGGCCTGAGGTTTTAC | | |
| | | | ATTTCCAACAAGCTGCCAGGTAAA | | |
| | | | GCCAATACATCTGTCCAGGAATCA | | |
| | | | CACTTTGCGTATCAAACGTCTA | | |
| | | | GATGACATTATCATTCCAAAGAGT | | |
| | | | TTCTTTTACAGGCTCTCAGATCAGT | | |
| | | | GTTCATCCACTACCTGACTAC | | |
| | | | TGTCATTCACAGGCATTCTGTTCCA | | |
| | | | CAGCAGGCCAGCTAACGTGGTATT | | |
| | | | TACAAACCTCACTCCTCTTAT | | |
| | | | ACAACAATCCAAGTGTTTCTTTTGT | | |
| | | | CAGTTGTCTGTGCCCCAGGAGATC | | |
| | | | CCTCTCTGCCTTGCCTTGCCC | | |
| | | | TCTGCCTTTGGAGACCAGCACCTC | | |
| | | | ATACTCAGTGAAGGCCTGGAGTGC | | |
| | | | TTAAGAGGGATTTCTTCCAGCT | | |
| | | | CTCTTGCCCTGGTCTTCAGTGTATT | | |
| | | | AGATGTATTACCTCCATGCTCTCAG | | |
| | | | TAGAGGCCCATAGGAAAGAG | | |
| | | | TAGGTAGGTTATGCCAGCTCACAC | | |
| | | | GCATCCTTTAAAAATGGTTTAGAA | | |
| | | | GTTTAGCTGGTTTCTTATTACT | | |
| | | | CCTGTCTATGGATGTTTCCTTCTGT | | |
| | | | CACTCTACTAGGGATGAAACAGCT | | |
| | | | AATCATGTTCAATAGTTACAT | | |
| | | | TTAGATTGGTTTTTAAAAACTATGA | | |
| | | | TTGTATTAGTTCGTTTCCATGCTGC | | |
| | | | TGATAAAGACATATCTGAGA | | |
| | | | CTGGAAACAAAAAGGGTTTAATTG | | |
| | | | GACTTACAGTTCCACATGGCTGGG | | |
| | | | GAGGCCTCAAAATCAGGTGGGA | | |
| | | | GGCAAAAGGTACTTCTTACGTGGT | | |
| | | | GGCATCAAGAGCAAAATGAGGAA | | |
| | | | GAAGCAAAAGCAGAAACTCTTCA | | |
| | | | TAAACCCACCAGATCTTGTGGGAC | | |
| | | | TTATTATCACGAGAATAGCACAGA | | |
| | | | AAAGACTGGCCTCCATGATTCA | | |
| | | | ATTACCTCCCACTGCGTCCCTCCCA | | |
| | | | CAACATGTGGGAATTCTGGGAGAT | | |
| | | | ACAATTCAAGTTGAGATTTGG | | |
| | | | GTGGGGACACAGCCAAACCATATC | | |
| | | | ATTCCTCCCTGGGCTCCTCCAAATT | | |
| | | | TCATAATCCTCACATTTCAAA | | |
| | | | ACCAATCATTCCTTCCCAACAGTTC | | |
| | | | CCCAAAGTCTTAACTCATTTCAGC | | |
| | | | ATTAACCCAAAAGTCCACAGT | | |
| | | | CCAAAGTCTCATCTGAGACAAGGC | | |
| | | | AAGTCCCTTCCACTTACAAGCCTG | | |
| | | | TAAAAGCAAGCTAGTTACCTCC | | |
| | | | TAGATACAATGGGGGGTACAGGTA | | |
| | | | TTGGGTAAATACAGCTGTTCCAAA | | |
| | | | TGAGAGAAATTGGCCAAAACAA | | |
| | | | AGGGGTTACAGGGTCCATCCAAGT | | |
| | | | CTGAAATCCAGTGGGGCAGTCAAA | | |
| | | | TTTTAAAGCTCCATAATGATCT | | |
| | | | CCTTTGACTCCATGTCTCACATTCA | | |
| | | | GGTCATGCTGATGCAAGAGATAGG | | |
| | | | TTCCCATGGTCTTGTGCAGCT | | |
| | | | CCGCCCCTGTGGCTTTGCAGAGTA | | |
| | | | CAGCCTCCCTCCTGGCTGCTTTCTC | | |
| | | | AGGCTGATGTTGAGTGTCTGT | | |
| | | | AGCTTTTCCAGGCACAAGATGCAA | | |
| | | | GTTGGTGGTTGATCTACCATTCTGG | | |
| | | | GGTCTACCATTCTGGGGTCTA | | |
| | | | CCGTTCTGGGACTGTGGCCTTCTTC | | |
| | | | TCACAGCTCCACTAGGCAGTGCCC | | |
| | | | CAACAGGGACTCTGTGTGGGG | | |
| | | | GCTCTGCCCCACATTTCCCTTCCAC | | |
| | | | ACTGCCCTAGGAGAGGTTCCCCAT | | |
| | | | GAGGGCTCTGCCCCTGCACCA | | |
| | | | AACTTTTGCCTGGACATCCAGGTG | | |
| | | | TTTCCATATATATTCTGAAATCTAG | | |
| | | | GCAGAGGTTCCCAAATCTCAA | | |
| | | | TTCTTGACATCTCTGCACCCACAG | | |
| | | | GCTCAACATCACATGGAAGCTGCC | | |
| | | | AATGCTTGGGGCCTCTACCCTC | | |
| | | | TGAAGCCACAGCCCAAGCTCTATG | | |
| | | | TTGGCTCCTTTCAGCCATGGCTGGA | | |
| | | | CCAGCTGGCACACAGGGCACC | | |
| | | | AAGTCCCTAGGCTGCACACAGCAC | | |
| | | | AGAGACCCTGGGCCCAGCCCACAA | | |
| | | | AACCACTTTTTCCTCCTGGGCC | | |
| | | | TCTGGGCCTGTGATGGGAGGGGCT | | |
| | | | GCCATGAAGGTCTCTGACATGACC | | |
| | | | TGGAGACATTTTCCCCATGGTC | | |
| | | | TTGGGGATTAACATTAGGCTCCTT | | |
| | | | GCTGCTTATGCAAATTTCTGCAGCC | | |
| | | | AGCTTGAATTTCTCCTTAAAA | | |
| | | | AAAATGGGTTTTTCTTTTCTACTGC | | |
| | | | ATCATCAGGCTGCAGATTTTCCAC | | |
| | | | ATTTATGCTCTTGTTTCCCTT | | |
| | | | TTAAAACAGAATGTTTTTAACAGC | | |
| | | | ACCCAAGTCACCTTTTGAATGCTTT | | |
| | | | GCTGCTTACAAATTTATTCCA | | |
| | | | CCAGATACCCTAAGTCATCTCTCTC | | |
| | | | AAGCTCTAAGTTCCACAAATCTCT | | |
| | | | AGGGCAAGGGTGAAATGCTGC | | |
| | | | CAGTCTCCTTGCTAAAACATAACA | | |
| | | | AGGGTCACCTTTACTTCAGTTCCCA | | |
| | | | ACAAGGTCTTCATCTCCATCT | | |
| | | | GAGACCACCTCAGCCTGGACCTTA | | |
| | | | TTGTTCATATCACTATCAGTATTTT | | |
| | | | TGTCAATGCCATTCACAGTCT | | |
| | | | CTAGGAGGTTCCAAACTTTCCTAC | | |
| | | | ATTTTCCTATCTTCTTCTGAGCCCT | | |
| | | | CCAGATTATTTCAACACCCAG | | |
| | | | TTCCAAAGTTGCTTCCACATTTTCG | | |
| | | | GGTATCTTTTCAGCAATGCCCCACT | | |
| | | | CTACTGGTACTATTAGTCCA | | |
| | | | TTTTCATGCTGCTGATAAAGACAT | | |
| | | | ACCTGAGACTGGGAACAAAAAGA | | |
| | | | GGTTTAATTGGACTTATACTTCC | | |
| | | | ACCTGGCTGGGGAGGCCTCAGAAT | | |
| | | | CATGGCAGGAGGTGAAAGGCATTT | | |
| | | | CTTACACGGCAGCAGCAAGAGA | | |
| | | | AAAATGAAGAAGCAGCAAAAGCA | | |
| | | | GAAACCCCTGATAAAACCATCAGA | | |
| | | | TCTCGTGAGACTTATTCACTATC | | |
| | | | ACAAGAATAGCATGGGAAAGACC | | |
| | | | AGCCCCCTTGATTCAATTACCTCCC | | |
| | | | CCTGGGTCCTGTGGGAATTCTG | | |
| | | | GAAGGTACAATTCAAGTTGAGATT | | |
| | | | TGGGTGGGGACACAGCCAAACCAT | | |
| | | | ATCAATGATTTTGTACTTTAAC | | |
| | | | CAGCTGAATGGAAGTACAATCTCT | | |
| | | | TGCTATATGACACAATAATTATTTG | | |
| | | | CAAAATCAGTAAACATATCAT | | |
| | | | AAGGAAATTATTTTTACAAGGTTT | | |
| | | | GAAACCTGAAATGCAGTCTATTAT | | |
| | | | CATACATAACTAAAAATAGAGC | | |
| | | | CTCAATAAACAGATTCCCAGTTTT | | |
| | | | GAAAATGCAACATTTGTACTCCAC | | |
| | | | ATTGTCAGTTTTCTTAGGTATA | | |
| | | | TTTATAAATACTCCTATAAAAATGT | | |
| | | | AAAGAAACACATAATGTAGATTGC | | |
| | | | TAATTTTATAATAACACAAGT | | |
| | | | TGATTTTGACATCCAACTTATTAAT | | |
| | | | TATGAAATGACTTTTGGCCTAGTA | | |
| | | | ACAATGAAAATGGGGGCAAAT | | |
| | | | ACAGATAAATGGTAATTCTTAGAA | | |
| | | | TGAACTACTCAGCACCAATTCTAA | | |
| | | | GTTTTTCTTGATGGTAAATCAT | | |
| | | | AATGTTCCCTTTCTCCTCGGTTCTG | | |
| | | | CAATCTATAGGCATACCATAATTG | | |
| | | | TAATCAATAGCTTAAAAATAT | | |
| | | | GTCTCTCTGTCCTATTCTGTATCTG | | |
| | | | TATCTCTTGGATTTTTACCTTTGCA | | |
| | | | ATAGTCAACTGAACCATCTT | | |
| | | | CTTGGAGTACTCATGAAGATGGAA | | |
| | | | GTCTACATGGAGAATACAGGATGA | | |
| | | | ATCCACTCTGTCTCCTGCAGTG | | |
| | | | AAGTCTGTTTGAAGGATGTATTTG | | |
| | | | GCTGTCTTCTGGACAGCCCATTCTA | | |
| | | | ATAACAGAAACAAACAAGTTA | | |
| | | | TTTTAAAACTTATTGGAATATTCAA | | |
| | | | ATATTAACCAAAGTAGAAAAATAT | | |
| | | | AATACACATCCATGTGCCCAT | | |
| | | | CACAGAACTTCACTGATTATCATC | | |
| | | | ATTTAGCCAGTCTTGAAGAAGCAA | | |
| | | | GTGCTAATTACAATCACAAATG | | |
| | | | AAACAAGATTCAGACTTCATGAAG | | |
| | | | AGCACTGCGCTATAATAAAAGAAG | | |
| | | | AAATGAGCACATACATTCTTTT | | |
| | | | ACTGACAGTCAAATGGTGAAGGTG | | |
| | | | GGCAGAATCATTATGTGATGCAAC | | |
| | | | ATGGCAAAAGTATACAGACAGT | | |
| | | | GCATCCAGAGGAAGGCACCTTGCT | | |
| | | | GAATGACTAGAATGGAAGTAGGA | | |
| | | | GACATTTTGCAGGCCCCCTTCAT | | |
| | | | CCTGCAGGGAGAACCAGAACCAC | | |
| | | | AGCAGCTCTATTTGCCTATTCCTCT | | |
| | | | TTAAATTACAAAGTTAAAATTT | | |
| | | | GGGAGTAGTAGAAAATCAATTGGT | | |
| | | | TATCTTATAGAGTCTCCTAGAATAT | | |
| | | | TTCATTGGCATTGAGAAGGTG | | |
| | | | GAAAATGCAAATTATATACTTTAA | | |
| | | | AATGTAATTTTTGCTTTTCACATAT | | |
| | | | GCTTAAAGCCTAAAACCTCTT | | |
| | | | AATAAACTTCTTCTGAAATATA | | |
| | | | (SEQ ID NO: 43) | | |
| | | NM_001164183.2 | ACGGAGACGGACCACAGCAAGCA | NP_001157655.1 | MESEELADRVLDVVER |
| | | | GAGGCTGGGGGGGGGAAAGACGA | | SLSNYPFDFQGARIITGQ |
| | | | GGAAAGAGGAGGAAAACAAAAGC | | EEGAYGWITI |
| | | | T | | NYLLGKFSQKTRWFSIV |
| | | | GCTACTTATGGAAGATACAAAGGA | | PYETNNQETFGALDLGG |
| | | | GTCTAACGTGAACACATTTTGCTC | | ASTQVIFVPQNQTIESP |
| | | | CAAGAATATCCTAGCCATCCTT | | DNALQFR |
| | | | GGCTTCTCCTCTATCATAGCTGTGA | | LYGKDYNVYTHSFLCY |
| | | | TAGCTTTGCTTGCTGTGGGGTTCAC | | GKDQALWQKLAKDIQV |
| | | | CCAGAACAAAGCATTGCCAG | | ASNEILRDPCFHPGYKK |
| | | | AAAACGTTAAGGATGGAAAGTGA | | VVNVSDLYK |
| | | | AGAGTTGGCAGACAGGGTTCTGGA | | TPCTKRFEMTLPFQQFEI |
| | | | TGTGGTGGAGAGGAGCCTCAGCA | | QGIGNYQQCHQSILELF |
| | | | ACTACCCCTTTGACTTCCAGGGTG | | NTSYCPYSQCAFNGIFL |
| | | | CCAGGATCATTACTGGCCAAGAGG | | PPLQGD |
| | | | AAGGTGCCTATGGCTGGATTAC | | FGAFSAFYFVMKFLNLT |
| | | | TATCAACTATCTGCTGGGCAAATT | | SEKVSQEKVTEMMKKF |
| | | | CAGTCAGAAAACAAGGTGGTTCAG | | CAQPWEEIKTSYAGVKE |
| | | | CATAGTCCCATATGAAACCAAT | | KYLSEYCF |
| | | | AATCAGGAAACCTTTGGAGCTTTG | | SGTYILSLLLQGYHFTA |
| | | | GACCTTGGGGGAGCCTCTACACAA | | DSWEHIHFIGKIQGSDA |
| | | | GTCACTTTTGTACCCCAAAACC | | GWTLGYMLNLTNMIPA |
| | | | AGACTATCGAGTCCCCAGATAATG | | EQPLSTPL |
| | | | CTCTGCAATTTCGCCTCTATGGCAA | | SHSTYVFLMVLFSLVLF |
| | | | GGACTACAATGTCTACACACA | | TVAIIGLLIFHKPSYFWK |
| | | | TAGCTTCTTGTGCTATGGGAAGGA | | DMV (SEQ ID NO: 46) |
| | | | TCAGGCACTCTGGCAGAAACTGGC | | |
| | | | CAAGGACATTCAGGTTGCAAGT | | |
| | | | AATGAAATTCTCAGGGACCCATGC | | |
| | | | TTTCATCCTGGATATAAGAAGGTA | | |
| | | | GTGAACGTAAGTGACCTTTACA | | |
| | | | AGACCCCCTGCACCAAGAGATTTG | | |
| | | | AGATGACTCTTCCATTCCAGCAGTT | | |
| | | | TGAAATCCAGGGTATTGGAAA | | |
| | | | CTATCAACAATGCCATCAAAGCAT | | |
| | | | CCTGGAGCTCTTCAACACCAGTTA | | |
| | | | CTGCCCTTACTCCCAGTGTGCC | | |
| | | | TTCAATGGGATTTTCTTGCCACCAC | | |
| | | | TCCAGGGGGATTTTGGGGCATTTT | | |
| | | | CAGCTTTTTACTTTGTGATGA | | |
| | | | AGTTTTTAAACTTGACATCAGAGA | | |
| | | | AAGTCTCTCAGGAAAAGGTGACTG | | |
| | | | AGATCATGAAAAAGTTCTGTGC | | |
| | | | TCAGCCTTGGGAGGAGATAAAAAC | | |
| | | | ATCTTACGCTGGAGTAAAGGAGAA | | |
| | | | CTACCTGAGTGAATACTGCTTT | | |
| | | | TCTGGTACCTACATTCTCTCCCTCC | | |
| | | | TTCTGCAAGGCTATCATTTCACAGC | | |
| | | | TGATTCCTGGGAGCACATCC | | |
| | | | ATTTCATTGGCAAGATCCAGGGCA | | |
| | | | GCGACGCCGGCTGGACTTTGGGCT | | |
| | | | ACATGCTGAACCTGACCAACAT | | |
| | | | GATCCCAGCTGAGCAACCATTGTC | | |
| | | | CACACCTCTCTCCCACTCCACCTAT | | |
| | | | GTCTTCCTCATGGTTCTATTC | | |
| | | | TCCCTGGTCCTTTTCACAGTGGCCA | | |
| | | | TCATAGGCTTGCTTATCTTTCACAA | | |
| | | | GCCTTCATATTTCTGGAAAG | | |
| | | | ATATGGTATAGCAAAAGCAGCTGA | | |
| | | | AATATGCTGGCTGGAGTGAGGAAA | | |
| | | | AAAATCGTCCAGGGAGCATTTT | | |
| | | | CCTCCATCGCAGTGTTCAAGGCCA | | |
| | | | TCCTTCCCTGTCTGCCAGGGCCAGT | | |
| | | | CTTGACGAGTGTGAAGCTTCC | | |
| | | | TTGGCTTTTACTGAAGCCTTTCTTT | | |
| | | | TGGAGGTATTCAATATCCTTTGCCT | | |
| | | | CAAGGACTTCGGCAGATACT | | |
| | | | GTCTCTTTCATGAGTTTTTCCCAGC | | |
| | | | TACACCTTTCTCCTTTGTACTTTGT | | |
| | | | GCTTGTATAGGTTTTAAACA | | |
| | | | CCTGACACCTTTCATAATCTTTGCT | | |
| | | | TTATAAAACAACAATATTGACTTT | | |
| | | | GTCTAGAAGAACTGAGAGTCT | | |
| | | | TGAGTCCTGTGATAGGAGGCTGAG | | |
| | | | CTGGCTGAAAGAAGAATCTCAGGA | | |
| | | | ACTGGTTCAGTTGTACTCTTTA | | |
| | | | AGAACCCCTTTCTCTCTCCTGTTTG | | |
| | | | CCATCCATTAAGAAAGCCATATGA | | |
| | | | TGCCTTTGGAGAAGGCAGACA | | |
| | | | CACATTCCATTCCCAGCCTGCTCTG | | |
| | | | TGGGTAGGAGAATTTTCTACAGTA | | |
| | | | GGCAAATATGTGCTAAAGCCA | | |
| | | | AAGAGTTTTATAAGGAAATATATG | | |
| | | | TGCTCATGCAGTCAATACAGTTCTC | | |
| | | | AATCCCACCCAAAGCAGGTAT | | |
| | | | GTCAATAAATCACATATTCCTAGG | | |
| | | | TGATACCCAAATGCTACAGAGTGG | | |
| | | | AACACTCAGACCTGAGATTTGC | | |
| | | | AAAAAGCAGATGTAAATATATGCA | | |
| | | | TTCAAACATCAGGGCTTACTATGA | | |
| | | | GGTAGGTGGTATATACATGTCA | | |
| | | | CAAATAAAAATACAGTTACAACTC | | |
| | | | AGGGTCACAAAAAATGCATCTTCC | | |
| | | | AATGCATATTTTTATTATGGTA | | |
| | | | AAATATACATAAATATAATTCACC | | |
| | | | ATTTTAACATTTAATTCATATTAAA | | |
| | | | TACGTACAAATCAGTGACATT | | |
| | | | TAGTACATTCACAGTGTTGTGCCA | | |
| | | | CCATCACCACTATTTACTTCCAGA | | |
| | | | ACATTTGCATCATCAATACATT | | |
| | | | GTCTAGAGACAAGACTATCCTGGG | | |
| | | | TAGGCAGAAACCATAGATCTTTTG | | |
| | | | TGTTTACAGCTATGGAAACCAA | | |
| | | | CTGTACCATAAAGATAGTTCACTG | | |
| | | | AGTTTTAAAGCCAAGCCACATCTT | | |
| | | | ATTTTTCCAAGGTTTAATTTAG | | |
| | | | TGAGAGGGCAGCATTAGTGTGGAG | | |
| | | | TGGCATGCTTTTGCCCTATCGTGGA | | |
| | | | ATTTACACATCAGAATGTGCA | | |
| | | | GGATCCAAGTCTGAAAGTGTTGCC | | |
| | | | ACCCGTCACACAACATGGGCTTTG | | |
| | | | TTTGCTTATTCCATGAAGCAGC | | |
| | | | AGCTATAGACCTTACCATGGAAAC | | |
| | | | ATGAAGAGACCCTGCACCCCTTTC | | |
| | | | CTTAAGGATTGCTGCAAGAGTT | | |
| | | | ACCTGTTGAGCAGGATTGACTGGT | | |
| | | | GATGTTTCATTCTGACCTTGTCCCA | | |
| | | | AGCTCTCCATCTCTAGATCTG | | |
| | | | GGGACTGACTGTTGAGCTGATGGG | | |
| | | | GAAAGAAAAGCTCTCACACAAACC | | |
| | | | GGAAGCCAAATGTCCCCTATCT | | |
| | | | CTTGAATGATCAAGTCACTTTTGAC | | |
| | | | AACATCCAGGTGAATATAAAAACT | | |
| | | | TAATAAAGCTGTGGAAAGGAA | | |
| | | | CTCTTAATCTTCTTTTCTGCTACTT | | |
| | | | AGGTTAAATTCACTAGATCTTGATT | | |
| | | | AGGAATCAAAATTCGAATTG | | |
| | | | GGACATGTTCAAATTCTTTCTTGTG | | |
| | | | GTAGTTGCCTATACTGTCATCGCTG | | |
| | | | CTGTTGGTTGAGCATTTGTG | | |
| | | | GTGTACCACGCTGTGTGCTCAAGG | | |
| | | | GTATTACATTCATCTTCTCATTTAA | | |
| | | | TCCTCACAACAATCTGAAGAA | | |
| | | | GGTAGGTATTACAATTCCCACTTC | | |
| | | | ATAGAAACAGAAACTGAGGTTCAG | | |
| | | | AGAGGTTAAGTCATTTGCCCAA | | |
| | | | ATGGCTGAGCCAAACCCTACCATG | | |
| | | | TACCTAACCTTTATTTTCTTTCCCG | | |
| | | | AACATACCAGGCTGTCTCCTC | | |
| | | | ATAACTTCCAAGCATGCACTTAAA | | |
| | | | ACTCCACATGAATACAAGOTTCAT | | |
| | | | GGGACTTGGTATTCATAGAAAG | | |
| | | | GGAGGCAGAAAGCTGGTCTGTTCC | | |
| | | | TGATAGGCTTGTAATTTAATATCAT | | |
| | | | TCTGTTCATGTGCTTTGGATG | | |
| | | | GAAGCACATCTGGCATATGATGCT | | |
| | | | AATCAGTGGTTCCCATACCCCTGG | | |
| | | | CTTCCTAATTTTAATGTTTGCT | | |
| | | | CACACCATAGTAGATTGACATCAA | | |
| | | | ATAGTGGCCGATGATGATGAAAAT | | |
| | | | AAAGGTCAAATAAGTTGAGCCAG | | |
| | | | ATAACAGCCGCTTTTTTCCTTCTGT | | |
| | | | CTGCCTATACAAAGCACTGTCATG | | |
| | | | CACACAATCTATTCTGACCCT | | |
| | | | CACAACAACCCATAAGGGTGTAAA | | |
| | | | TAGTATTTCCATTTTACAAATGAGG | | |
| | | | ATCACACAAACTACTACATGG | | |
| | | | CAGAGCAGATACTCCAACTCATGT | | |
| | | | CTTCTGGTTGAAGCCTATTGCTTTT | | |
| | | | TCTTTTCTAAACACTTTCCCT | | |
| | | | CAGCAAGTTGGAATTAGACTTCAC | | |
| | | | AAGTCTCCTTCAGAGAACACAAAT | | |
| | | | CTTTTCTTATTCCATTCCTGTT | | |
| | | | TGGTTGCCTACGTCCAATCTCCCCC | | |
| | | | TCCCCAGAGATGCCAAAAAAAAA | | |
| | | | ATCCTTTAAGGTATTTGGGAGC | | |
| | | | CAAACTCAACTTGTTAAAATCTCA | | |
| | | | AATTATGGAGACAATCAGCAGACA | | |
| | | | CAACCTAACCCCAATTATTTTG | | |
| | | | GCAGGAAGGTTGGTTTAGAGGCAG | | |
| | | | ATCCAGCAATCTGCTTTGGGCCAC | | |
| | | | TCTGGGTGGGGTAGGTGAAATA | | |
| | | | AGATTGGTCACTGTTAACTAATTTT | | |
| | | | AATATTGGATTGGCCATTGGTTATC | | |
| | | | ACTGATTACCATTCTCCCCT | | |
| | | | GGATTTTCACCCAGGACTCAAAAC | | |
| | | | TTGGTTCTGCTAACCCTGTTCCTTT | | |
| | | | ATGAGGAACCTTTTAAAGATT | | |
| | | | CCTTTATAAGGTGGGAGTTTTTTTT | | |
| | | | CTATGAACCTATAGGGGAGAAAAA | | |
| | | | AGATCAGCAGAAGTCATTACT | | |
| | | | TTTTTTTTTTTTTTTTTTTTTTTTTGA | | |
| | | | GAGAGAGTCTCACTCCATTGCCCA | | |
| | | | GGCTGGAGTGCAGTGGTGC | | |
| | | | TATCTCGGCTCACTGCAACCTCCG | | |
| | | | CCTCCTGGCTTCAACCAATTCTCCT | | |
| | | | GCCTCAGCCTCCCGAGTAGCT | | |
| | | | GGGATTGCAGGTGCCCACCACCAC | | |
| | | | ACCCGGCTAATTTTTGTATTTTTAG | | |
| | | | TAAAGACAGGGTTTCACCATG | | |
| | | | TTGGCCAGGCTGGTCTCCAACTCC | | |
| | | | CAATCTCAGGTGATCCTATTGCCTC | | |
| | | | GGGCTCCCAAAGTGCTGGGAT | | |
| | | | TACAGGAGTGAGCCACCATGCCTG | | |
| | | | GCCAGAAGTGGTTACTTCTGTAGA | | |
| | | | CAAAAGAATAATGCTACTTAAT | | |
| | | | CAGGCTTTCTGTGTGACAAGAAAG | | |
| | | | AGAAAGAAAATAAAGAAGTTTCA | | |
| | | | ATTCATCCAATTCTTAATAAGAA | | |
| | | | ATATGTAAATAAAATTTTTTAAAA | | |
| | | | TTACACTTCATTTTAATGTTGTATC | | |
| | | | AGTCAAGGTCCCTGCAAGAGA | | |
| | | | TGGATGGTATGGTACACTCAAACT | | |
| | | | GGGTAACACAGGAGAGTTTTCACA | | |
| | | | AAGCAACTAAATCCAAAATACT | | |
| | | | ATCAAGGAATCAATATAAAAATTG | | |
| | | | TTAATATTTTTCTCATACTAAATTT | | |
| | | | TCAAAATATTTTGTGTCTATT | | |
| | | | ACATTTACAGCACATCTTAATTAG | | |
| | | | GACTAGCTGTGTGTTCACCTCACAT | | |
| | | | GTGGCTTGTAGCTACCATACT | | |
| | | | GGACAGCACATGTCCAAAAAAATA | | |
| | | | CACGTAAAGTTAAAGTTTAAAAGA | | |
| | | | CACAGGAACTAAGCCCTCATTG | | |
| | | | TCTTTCCCTTGGGAGGTAGTTTAAA | | |
| | | | GAGCTATAGATGCTGTAACATTCT | | |
| | | | TGCTATTATTTATTATATATG | | |
| | | | ACATTATTCCTAAAAAAGCTTTTG | | |
| | | | AGATCCTAGGTTGTATTCCTCAGGT | | |
| | | | TTTGTTGCCTTCCCATGAAGA | | |
| | | | TGTGAAGGCAGGGATGCCTGTTAT | | |
| | | | TCAGTCCAAGATGCATGACAAGAG | | |
| | | | ACCTTGGGAAAGTTTCATCTGG | | |
| | | | ATTTAAAGATTAATTCTTCATGCTT | | |
| | | | ACATTCCATACTCAAAATGTAAAT | | |
| | | | TTGAATATTAAAATAAAGATG | | |
| | | | ATTTTTTTTTTGGAGCTAGTCTTGC | | |
| | | | TCTGTTGCCCAGGCTGGAATGCAG | | |
| | | | TGGCATGATCATGGCTCACTG | | |
| | | | CAGCCTCGACCTCCCAAGCTCAAG | | |
| | | | CAAGGCTACAGGTGTGCACCTAAG | | |
| | | | TAGCTAGGACTACAGGTGTGCA | | |
| | | | CCACCATGTCTAGCTATTTTTTTTT | | |
| | | | CTGTAGAGACAGGGTTTTCCTATG | | |
| | | | TTGTCCAGGCTGGTCTCGAAC | | |
| | | | TCCTGCCCTCAAGCAATCCTCCTGC | | |
| | | | CTTGGCCTCCCAAAGTGTTGAGAT | | |
| | | | TACAGGCGTAAGCCACTGCAC | | |
| | | | CTGGCCAAGATGAATATTTTAATA | | |
| | | | GCTCACAGAACAAAGTTTGCCACA | | |
| | | | TAATGATAAAATTACTATGAAA | | |
| | | | ATATATTCCCTTTATTGTCAGTTTA | | |
| | | | AAAGATGAACTGAGTTTCACCCAA | | |
| | | | ACTGGTCTGGCCCCTCTCTGA | | |
| | | | TTCAAATACCAATAGTTGCTCTGAT | | |
| | | | TCAAATTCCAACTGTTAGAACATG | | |
| | | | ACAGCTGCTCATAACTAGCTT | | |
| | | | TGCTTACTAACCATGTTTCTTTCCA | | |
| | | | TTTGTATTAGGTCCTTTACTTTTTA | | |
| | | | TAACAGCCTCAAAGTTTCAT | | |
| | | | GAATTGCTGCAGTAAACATTGATT | | |
| | | | TTCATGTTTGTGAGTCTGCAAGCCA | | |
| | | | GCTGGGCAGCTCTACTTCAGG | | |
| | | | TGGTAAGGGTGGATCAGACCTATT | | |
| | | | CCATATACCTCTTGTTCTCCTTGTC | | |
| | | | CAGTGGTTTCTAGGGATATGT | | |
| | | | TCTCATGATGAACCCCCCAGAGGC | | |
| | | | TCGTGAAAGTGAGAGGAAACTAGG | | |
| | | | ATGCCTCTTAAGGTCTTGGTCA | | |
| | | | GGATGGGGTCTCCTGTCACTTCTGT | | |
| | | | CACAGGCTATTGTAAGTCATATGA | | |
| | | | GCAAGCTCAATAAAATATAAA | | |
| | | | CAAGTCAGATAAACAGTGGGAGG | | |
| | | | AATGGCAAAGTCATATGGCCAAGG | | |
| | | | CCATGAGTGATTAATTTTAACAC | | |
| | | | AGGAAAAAAGTAAAGCATTAAAT | | |
| | | | GCGATTATTTAATATACAATGTCTT | | |
| | | | ATTAACTGAAATATAAAATGTG | | |
| | | | TTTACTGTAAAATATAATCTGTTTA | | |
| | | | TCTCACCAAAGAAATATTATCTTTA | | |
| | | | AAAAATGTCATTACTTCTAA | | |
| | | | GACATCATCAGTCTGCAACTTCTTT | | |
| | | | CCATAGCCTTAATCAGGATGGTGT | | |
| | | | GGCAGCTCCCACATTACCCTC | | |
| | | | GCATTCTAAACTGGTAGATGTCCT | | |
| | | | AGGAAACCATACATCTATGTATTT | | |
| | | | TTCTTATTTTATACGTTTAGGA | | |
| | | | CAATGTATAGCTAATTACCCAACT | | |
| | | | TTTTATTTGCATACAAATCTAATAC | | |
| | | | AACTGAACACAATCAGTTTTA | | |
| | | | TCACAGGTATAATGGATTTTTCAAT | | |
| | | | AGTGAGGAGGTGCCTCCATGAGCC | | |
| | | | TTCTCTTTAGAAAAGTGGCAT | | |
| | | | TCAAGACTCTTCATTTGAAGTGAA | | |
| | | | GATTGCTATGTCTTTTGCATTGCTC | | |
| | | | TATTTTACATAAATTAAGTTA | | |
| | | | TAAATTGACACTATAATCAACTGA | | |
| | | | CACCATGATCAGTGATGATGATCA | | |
| | | | CCCTCATCAGCACTAGAGTTGA | | |
| | | | CTTGTTTTTATAACCCCTTTGCATG | | |
| | | | TATGTTGAATAGCAAAGTTCATCA | | |
| | | | GAGAACATGTATTAGTCAATG | | |
| | | | GTAACTAAGATACTCTCATCTAAG | | |
| | | | AAATAACATCACCTCTTCTAATGA | | |
| | | | AGTTCTAAGAAGAGAGGGAAGA | | |
| | | | AAAAGTCTTGGGAGCTAGTCAGGG | | |
| | | | AATAGTGTCTATTTGCAATTACCTA | | |
| | | | AACTGAACTCTACCATTACTC | | |
| | | | CTAACCCAGTTCCTCCTCCTGTGTT | | |
| | | | TTACATGATTAATGCCACCCCTGC | | |
| | | | CTCAATGAACCAAGATCAGCT | | |
| | | | CCATCACTGGGACCTCCCCATTCT | | |
| | | | GCCTGTGCAATATTTTTCTTTTTTA | | |
| | | | TTTCTCCTTCTAATATTACTG | | |
| | | | TTATTGCTCCAGTAAAGAGCTGTA | | |
| | | | ATATATTTTACCTGGACTCATACCA | | |
| | | | GGAATGGTGGTGTTGCTTCCA | | |
| | | | ATCTGTTGCTGCTAGATTAATCTTT | | |
| | | | GCAAAGCACAGGCTTAATTTCATT | | |
| | | | GCTGCTCAACTAAAACCACTG | | |
| | | | GTGGCTTTCCATTGCCTACAAAAT | | |
| | | | AAAGTCAACCTCCCCATCAGACAT | | |
| | | | TCAAGGCTTTCAATGATCCATG | | |
| | | | GCCGCCAGCTCTCTCCAGGCTCAT | | |
| | | | ATCCCACTCCACTCCTCTGATGTTT | | |
| | | | CCTACACTACACTACACTATA | | |
| | | | CTACACTACAGCCAGGTAGAATGA | | |
| | | | CTGTTCACCCAACACCACTCAGGT | | |
| | | | TGTCTTCTCAACTTGCAATACT | | |
| | | | CTTGCACCTTCAAAGCTCATTTCAA | | |
| | | | ATGCCCCTTCATTTGTGAAGCCTTC | | |
| | | | TCCAAATTTCCAAGTCAGAA | | |
| | | | TGTCTCTTCCTTGTGCTACCACAAC | | |
| | | | CCTTTAACTGAGCCTCCATTAGTGC | | |
| | | | ACTGAGACCATTCTGTTCAG | | |
| | | | TGTCTGGGTGAAGCTTCCTGGTGA | | |
| | | | AAAATATGTTACCTATTTCTTTCTG | | |
| | | | AAAACTTGGATTCAGGGATAT | | |
| | | | TATCACGGACCTAAGGTAATAGTT | | |
| | | | CTAGCCAACCTCCCTGTCCACTGC | | |
| | | | CAGGCCGACTACAAACCCTTCT | | |
| | | | GTTGCTGGCGAGCTGGTCCGCACC | | |
| | | | ACTAGTTCTGCTTCACTCTATTTAT | | |
| | | | CTCTTGATGTAACCATCTTCT | | |
| | | | TTCTCCAGGTTTTAAGAACCAGCC | | |
| | | | CAACTCCTGGTTCCCTGATGAAGC | | |
| | | | TTTTATTCCCCTAGCCACATGG | | |
| | | | AACTTTTCCTTTTTGGAACATGCCT | | |
| | | | TTAGTTTCTGTGTAGTTTGCCATGC | | |
| | | | AGCACTTCATTGTACACATT | | |
| | | | ATTAAAACAGAATTTTAAGGATTA | | |
| | | | GAATGAACCTTAAAAGATCATGCA | | |
| | | | TCTCAAAATTTAATGTACATAC | | |
| | | | AAATTACCCAGGGATTTTGTTGAA | | |
| | | | ATAAAAATTATTTAATTTTAATTAA | | |
| | | | TATAAATAATTCAGTAGGTCT | | |
| | | | GGGGTGAGGCCTGAGGTTTTACAT | | |
| | | | TTCCAACAAGCTGCCAGGTAAAGC | | |
| | | | CAATACATCTGTCCAGGAATCA | | |
| | | | CACTTTGCGTATCAAAGGTCTAGA | | |
| | | | TGACATTATCATTCCAAAGAGTTTC | | |
| | | | TTTTACAGGCTCTCAGATCAG | | |
| | | | TGTTCATCCACTACCTGACTACTGT | | |
| | | | CATTCACAGGCATTCTGTTCCACA | | |
| | | | GCAGGCCAGCTAACGTGGTAT | | |
| | | | TTACAAAGCTCACTCCTCTTATACA | | |
| | | | ACAATCCAAGTGTTTCTTTTGTCAG | | |
| | | | TTGTCTGTGCCCCAGGAGAT | | |
| | | | CCCTCTCTGCCTTGCCTTGCCCTCT | | |
| | | | GCCTTTGGAGACCAGCACCTCATA | | |
| | | | CTCAGTGAAGGCCTGGAGTGC | | |
| | | | TTAAGAGGGATTTCTTCCAGCTCTC | | |
| | | | TTGCCCTGGTCTTCAGTGTATTAGA | | |
| | | | TGTATTACCTCCATGCTCTC | | |
| | | | AGTAGAGGCCCATAGGAAAGAGT | | |
| | | | AGGTAGGTTATGCCAGCTCACACG | | |
| | | | CATCCTTTAAAAATGGTTTAGAA | | |
| | | | GTTTAGCTGGTTTCTTATTACTCCT | | |
| | | | GTCTATGGATGTTTCCTTCTGTCAC | | |
| | | | TTCTACTAGGGATGAAACAGC | | |
| | | | TAATCATGTTCAATAGTTACATTTA | | |
| | | | GATTGGTTTTTAAAAACTATGATTG | | |
| | | | TATTAGTTCGTTTCCATGCT | | |
| | | | GCTGATAAAGACATATCTGAGACT | | |
| | | | GGAAACAAAAAGGGTTTAATTGGA | | |
| | | | CTTACAGTTCCACATGGCTGGG | | |
| | | | GAGGCCTCAAAATCAGGTGGGAGG | | |
| | | | CAAAAGGTACTTCTTACGTGGTGG | | |
| | | | CATCAAGACCAAAATGAGGAAG | | |
| | | | AAGCAAAAGCAGAAACTCTTCATA | | |
| | | | AACCCACCAGATCTTGTGGGACTT | | |
| | | | ATTATCACGAGAATAGCACAGA | | |
| | | | AAAGACTGGCCTCCATGATTCAAT | | |
| | | | TACCTCCCACTGCGTCCCTCCCACA | | |
| | | | ACATGTGGGAATTCTGGGAGA | | |
| | | | TACAATTCAAGTTGAGATTTGGGT | | |
| | | | GGGGACACAGCCAAACCATATCAT | | |
| | | | TCCTCCCTGGGCTCCTCCAAAT | | |
| | | | TTCATAATCCTCACATTTCAAAACC | | |
| | | | AATCATTCCTTCCCAACAGTTCCCC | | |
| | | | AAAGTCTTAACTCATTTCAG | | |
| | | | CATTAACCCAAAAGTCCACAGTCC | | |
| | | | AAAGTCTCATCTGACACAAGCCAA | | |
| | | | CTCCCTTCCACTTACAAGCCTG | | |
| | | | TAAAAGCAAGCTAGTTACCTCCTA | | |
| | | | GATACAATGGGGGGTACAGGTATT | | |
| | | | GGGTAAATACAGCTGTTCCAAA | | |
| | | | TGAGAGAAATTGGCCAAAACAAA | | |
| | | | GGGGTTACAGGGTCCATGCAAGTC | | |
| | | | TGAAATCCAGTGGGGCAGTCAAA | | |
| | | | TTTTAAAGCTCCATAATGATCTCCT | | |
| | | | TTGACTCCATGTCTCACATTCAGGT | | |
| | | | CATGCTGATGCAAGAGATAG | | |
| | | | GTTCCCATGGTCTTGTGCAGCTCCG | | |
| | | | CCCCTGTGGCTTTGCAGAGTACAG | | |
| | | | CCTCCCTCCTGGCTGCTTTCT | | |
| | | | CAGGCTGATGTTGAGTGTCTGTAG | | |
| | | | CTTTTCCAGGCACAAGATGCAAGT | | |
| | | | TGGTGGTTGATCTACCATTCTG | | |
| | | | GGGTCTACCATTCTGGGGTCTACC | | |
| | | | GTTCTGGGACTGTGGCCTTCTTCTC | | |
| | | | ACAGCTCCACTAGGCAGTGCC | | |
| | | | CCAACAGGGACTCTGTGTGGGGGC | | |
| | | | TCTGCCCCACATTTCCCTTCCACAC | | |
| | | | TGCCCTAGGAGAGGTTCCCCA | | |
| | | | TGAGGGCTCTGCCCCTGCAGCAAA | | |
| | | | CTTTTGCCTGGACATCCAGGTGTTT | | |
| | | | CCATATATATTCTGAAATCTA | | |
| | | | GGCAGAGGTTCCCAAATCTCAATT | | |
| | | | CTTGACATCTCTGCACCCACAGGC | | |
| | | | TCAACATCACATGGAAGCTGCC | | |
| | | | AATGCTTGGGGCCTCTACCCTCTG | | |
| | | | AAGCCACAGCCCAAGCTCTATGTT | | |
| | | | GGCTCCTTTCAGCCATGGCTGG | | |
| | | | AGCAGCTGGGACACAGGGCACCA | | |
| | | | AGTCCCTAGGCTGCACACAGCACA | | |
| | | | GAGACCCTGGGCCCAGCCCACAA | | |
| | | | AACCACTTTTTCCTCCTGGGCCTCT | | |
| | | | GGGCCTGTGATGGGAGGGGCTGCC | | |
| | | | ATGAAGGTCTCTGACATGACC | | |
| | | | TGGAGACATTTTCCCCATGGTCTTG | | |
| | | | GGGATTAACATTAGGCTCCTTGCT | | |
| | | | GCTTATGCAAATTTCTGCAGC | | |
| | | | CAGCTTGAATTTCTCCTTAAAAAA | | |
| | | | AATGGGTTTTTCTTTTCTACTGCAT | | |
| | | | CATCAGGCTGCAGATTTTCCA | | |
| | | | CATTTATGCTCTTGTTTCCCTTTTA | | |
| | | | AAACAGAATGTTTTTAACAGCACC | | |
| | | | CAAGTCACCTTTTGAATGCTT | | |
| | | | TGCTGCTTAGAAATTTATTCCACCA | | |
| | | | GATACCCTAAGTCATCTCTCTCAA | | |
| | | | GCTCTAAGTTCCACAAATCTC | | |
| | | | TAGGGCAAGGGTGAAATGCTGCCA | | |
| | | | GTCTCCTTGCTAAAACATAACAAG | | |
| | | | GGTCACCTTTACTTCAGTTCCC | | |
| | | | AACAAGGTCTTCATCTCCATCTGA | | |
| | | | GACCACCTCAGCCTGGACCTTATT | | |
| | | | GTTCATATCACTATCAGTATTT | | |
| | | | TTGTCAATGCCATTCACAGTCTCTA | | |
| | | | GGAGGTTCCAAACTTTCCTACATTT | | |
| | | | TCCTATCTTCTTCTGAGCCC | | |
| | | | TCCAGATTATTTCAACACCCAGTTC | | |
| | | | CAAAGTTGCTTCCACATTTTCGGGT | | |
| | | | ATCTTTTCAGCAATGCCCCA | | |
| | | | CTCTACTGGTACTATTAGTCCATTT | | |
| | | | TCATGCTGCTGATAAAGACATACC | | |
| | | | TGAGACTGGGAACAAAAAGAG | | |
| | | | GTTTAATTGGACTTATAGTTCCACC | | |
| | | | TGGCTGGGGAGGCCTCAGAATCAT | | |
| | | | GGCAGGAGGTGAAAGGCATTT | | |
| | | | CTTACACGGCAGCAGCAAGAGAA | | |
| | | | AAATGAAGAAGCAGCAAAAGCAG | | |
| | | | AAACCCCTCATAAAACCATCAGAT | | |
| | | | CTCGTGAGACTTATTCACTATCACA | | |
| | | | AGAATAGCATGGGAAAGACCAGC | | |
| | | | CCCCTTGATTCAATTACCTCCC | | |
| | | | CCTGGGTCCTGTGGGAATTCTGGA | | |
| | | | AGGTACAATTCAAGTTGAGATTTG | | |
| | | | GGTGGGGACACAGCCAAACCAT | | |
| | | | ATCAATGATTTTGTACTTTAACCAG | | |
| | | | CTGAATGGAAGTACAATCTCTTGC | | |
| | | | TATATGACACAATAATTATTT | | |
| | | | CCAAAATGAGTAAACATATCATAA | | |
| | | | GGAAATTATTTTTACAAGGTTTGA | | |
| | | | AACCTGAAATGCAGTCTATTAT | | |
| | | | CATACATAACTAAAAATAGAGCCT | | |
| | | | CAATAAACAGATTCCCAGTTTTGA | | |
| | | | AAATGCAACATTTGTACTCCAC | | |
| | | | ATTGTCAGTTTTCTTAGGTATATTT | | |
| | | | ATAAATACTCCTATAAAAATGTAA | | |
| | | | AGAAACACATAATGTAGATTG | | |
| | | | CTAATTTTATAATAACACAAGTTG | | |
| | | | ATTTTGACATCCAACTTATTAATTA | | |
| | | | TGAAATGACTTTTGGCCTAGT | | |
| | | | AACAATGAAAATGGGGGCAAATA | | |
| | | | CAGATAAATGGTAATTCTTAGAAT | | |
| | | | GAACTACTCAGCACCAATTCTAA | | |
| | | | GTTTTTCTTGATGGTAAATCATAAT | | |
| | | | GTTCCCTTTCTCCTCGGTTCTGCAA | | |
| | | | TCTATAGGCATACCATAATT | | |
| | | | GTAATCAATAGCTTAAAAATATGT | | |
| | | | CTCTCTGTCCTATTCTGTATCTGTA | | |
| | | | TCTCTTGGATTTTTACCTTTG | | |
| | | | CAATAGTCAACTGAACCATCTTCTT | | |
| | | | GGAGTACTCATGAAGATGGAAGTC | | |
| | | | TACATGGAGAATACAGGATGA | | |
| | | | ATCCACTCTGTCTCCTGCAGTGAA | | |
| | | | GTCTGTTTGAAGGATGTATTTGGCT | | |
| | | | GTCTTCTGGACAGGCCATTCT | | |
| | | | AATAACAGAAACAAACAAGTTATT | | |
| | | | TTAAAACTTATTGGAATATTCAAA | | |
| | | | TATTAACCAAAGTAGAAAAATA | | |
| | | | TAATACACATCCATGTGCCCATCA | | |
| | | | CAGAACTTCACTGATTATCATCATT | | |
| | | | TAGCCAGTCTTGAAGAAGCAA | | |
| | | | GTGCTAATTACAATCACAAATGAA | | |
| | | | ACAAGATTCAGACTTCATGAAGAG | | |
| | | | CACTGCGCTATAATAAAAGAAG | | |
| | | | AAATGAGCACATACATTCTTTTACT | | |
| | | | GACAGTCAAATGGTGAAGGTGGGC | | |
| | | | AGAATCATTATGTGATGCAAC | | |
| | | | ATGGCAAAAGTATACAGACAGTGC | | |
| | | | ATCCAGAGGAAGCCACCTTGCTGA | | |
| | | | ATGACTAGAATGGAAGTAGGAG | | |
| | | | ACATTTTGCAGGCCCCCTTCATCCT | | |
| | | | GCAGGGAGAACCAGAACCACAGC | | |
| | | | AGCTCTATTTGCCTATTCCTCT | | |
| | | | TTAAATTACAAAGTTAAAATTTGG | | |
| | | | GAGTAGTAGAAAATCAATTGGTTA | | |
| | | | TCTTATAGAGTCTCCTAGAATA | | |
| | | | TTTCATTGGCATTGAGAAGGTGGA | | |
| | | | AAATGCAAATTATATACTTTAAAA | | |
| | | | TGTAATTTTTGCTTTTCACATA | | |
| | | | TGCTTAAAGCCTAAAACCTCTTAA | | |
| | | | TAAACTTCTTCTGAAATATA (SEQ | | |
| | | | ID NO: 45) | | |
| | | NM_001312654.1 | CCTGTTGCTCTTTGCTCTAATGAGC | NP_001299583.1 | MERAREVIPRSQHQETP |
| | | | CTTGAGAAAGGATTGCTGGTCATG | | VYLGATAGMRLLRMES |
| | | | GGACCAGAGGCTTTATGGGGA | | EELADRVLDVV |
| | | | GGGAAGAACTGTTCTTGACTTTCA | | ERSLSNYPFDFQGARIIT |
| | | | GTTTTTCGAGCGGGTTTCAAGGTA | | GQEEGAYGWITINYLLG |
| | | | CAAAATTCAGTAGGACACGACT | | KFSQKTRWFSIVPYETN |
| | | | TTCTGAGTCATATGCTGGATTTGAG | | NQETFG |
| | | | GAGATACTGAAGCCACAAGACTGA | | ALDLGGASTQVTFVPQ |
| | | | AATAACTTTTAAACTGTGGGT | | NQTIESPDNALQFRLYG |
| | | | GATTTGGCTAAAAGCTACTCTCAT | | KDYNVYTHSFLCYGKD |
| | | | CTATTATATATTCATCTTACTAGTT | | QALWQKLAK |
| | | | TTGCTCTCAGAGGAAGTTCCT | | DIQVASNEILRDPCFHPG |
| | | | GTTTCCAAGTATGGGATTGTGCTG | | YKKVVNVSDLYKTPCT |
| | | | GATGCGGGTTCTTCTCACACAAGT | | KRFEMTLPFQQFEIQGIG |
| | | | TTATACATCTATAAGTGGCCAG | | NYQQCH |
| | | | CAGAAAAGGAGAATGACACAGGC | | QSILELENTSYCPYSQCA |
| | | | GTGGTGCATCAAGTAGAAGAATGC | | FNGIFLPPLQGDFGAFSA |
| | | | AGGGTTAAAGGTCCTGGAATCTC | | FYFVMKFLNLTSEKVSQ |
| | | | AAAATTTGTTCAGAAAGTAAATGA | | EKVTE |
| | | | AATAGGCATTTACCTGACTGATTG | | MMKKFCAQPWEEIKTS |
| | | | CATGGAAAGAGCTAGGGAAGTG | | YAGVKEKYLSEYCFSG |
| | | | ATTCCAAGGTCCCAGCACCAAGAG | | TYILSLLLQGYHFTADS |
| | | | ACACCCGTTTACCTGGGAGCCACG | | WEHIHFIGK |
| | | | GCAGGCATGCGGTTGCTCAGGA | | IQGSDAGWILGYMLNL |
| | | | TGGAAAGTGAAGAGTTGGCAGACA | | TNMIPAEQPLSTPLSHST |
| | | | GGGTTCTGGATGTGGTGGAGAGGA | | YVFLMVLFSLVLFTVAII |
| | | | GCCTCACCAACTACCCCTTTGA | | GLLIFH |
| | | | CTTCCAGGGTGCCAGGATCATTAC | | KPSYFWKDMV (SEQ ID |
| | | | TGGCCAAGAGGAAGGTCCCTATGG | | NO: 48) |
| | | | CTGGATTACTATCAACTATCTG | | |
| | | | CTGGGCAAATTCAGTCAGAAAACA | | |
| | | | AGGTGGTTCAGCATAGTCCCATAT | | |
| | | | GAAACCAATAATCAGGAAACCT | | |
| | | | TTGGAGCTTTGGACCTTGGGGGAG | | |
| | | | CCTCTACACAAGTCACTTTTGTACC | | |
| | | | CCAAAACCAGACTATCGAGTC | | |
| | | | CCCAGATAATGCTCTGCAATTTCG | | |
| | | | CCTCTATGGCAAGGACTACAATGT | | |
| | | | CTACACACATAGCTTCTTGTGC | | |
| | | | TATGGGAAGGATCAGGCACTCTGG | | |
| | | | CAGAAACTGGCCAAGGACATTCAG | | |
| | | | GTTGCAAGTAATGAAATTCTCA | | |
| | | | GGGACCCATGCTTTCATCCTGGAT | | |
| | | | ATAAGAAGGTAGTGAACGTAAGTG | | |
| | | | ACCTTTACAAGACCCCCTGCAC | | |
| | | | CAAGAGATTTGAGATGACTCTTCC | | |
| | | | ATTCCAGCAGTTTGAAATCCAGGG | | |
| | | | TATTGGAAACTATCAACAATGC | | |
| | | | CATCAAAGCATCCTGGAGCTCTTC | | |
| | | | AACACCAGTTACTGCCCTTACTCC | | |
| | | | CAGTGTGCCTTCAATGGGATTT | | |
| | | | TCTTGCCACCACTCCAGGGGGATT | | |
| | | | TTGGGGCATTTTCAGCTTTTTACTT | | |
| | | | TGTGATGAAGTTTTTAAACTT | | |
| | | | GACATCAGAGAAAGTCTCTCAGGA | | |
| | | | AAAGGTGACTGAGATGATGAAAA | | |
| | | | AGTTCTGTGCTCAGCCTTGGGAG | | |
| | | | GAGATAAAAACATCTTACGCTGGA | | |
| | | | GTAAAGGAGAAGTACCTGAGTGAA | | |
| | | | TACTGCTTTTCTGGTACCTACA | | |
| | | | TTCTCTCCCTCCTTCTGCAAGGCTA | | |
| | | | TCATTTCACAGCTGATTCCTGGGA | | |
| | | | GCACATCCATTTCATTGGCAA | | |
| | | | GATCCAGGGCAGCGACGCCGGCTG | | |
| | | | GACTTTGGGCTACATGCTGAACCT | | |
| | | | GACCAACATGATCCCACCTGAG | | |
| | | | CAACCATTGTCCACACCTCTCTCCC | | |
| | | | ACTCCACCTATGTCTTCCTCATGGT | | |
| | | | TCTATTCTCCCTGGTCCTTT | | |
| | | | TCACAGTGGCCATCATAGGCTTGC | | |
| | | | TTATCTTTCACAAGCCTTCATATTT | | |
| | | | CTGGAAAGATATGGTATAGCA | | |
| | | | AAAGCAGCTGAAATATGCTGGCTG | | |
| | | | GAGTGAGGAAAAAAATCGTCCAG | | |
| | | | GGAGCATTTTCCTCCATCGCAGT | | |
| | | | GTTCAAGGCCATCCTTCCCTGTCTG | | |
| | | | CCAGGGCCAGTCTTGACGAGTGTG | | |
| | | | AAGCTTCCTTGGCTTTTACTG | | |
| | | | AAGCCTTTCTTTTGGAGGTATTCAA | | |
| | | | TATCCTTTGCCTCAAGGACTTCGGC | | |
| | | | AGATACTGTCTCTTTCATGA | | |
| | | | GTTTTTCCCAGCTACACCTTTCTCC | | |
| | | | TTTGTACTTTGTGCTTGTATAGGTT | | |
| | | | TTAAAGACCTGACACCTTTC | | |
| | | | ATAATCTTTGCTTTATAAAAGAAC | | |
| | | | AATATTGACTTTGTCTAGAAGAAC | | |
| | | | TGAGAGTCTTGAGTCCTGTGAT | | |
| | | | AGGAGGCTGAGCTGGCTGAAAGA | | |
| | | | AGAATCTCAGGAACTGGTTCAGTT | | |
| | | | GTACTCTTTAAGAACCCCTTTCT | | |
| | | | CTCTCCTGTTTGCCATCCATTAAGA | | |
| | | | AAGCCATATGATGCCTTTGGAGAA | | |
| | | | GGCAGACACACATTCCATTCC | | |
| | | | CAGCCTGCTCTGTGGGTAGGAGAA | | |
| | | | TTTTCTACAGTAGGCAAATATGTG | | |
| | | | CTAAAGCCAAAGAGTTTTATAA | | |
| | | | GGAAATATATGTGCTCATGCAGTC | | |
| | | | AATACAGTTCTCAATCCCACCCAA | | |
| | | | AGCAGGTATGTCAATAAATCAC | | |
| | | | ATATTCCTAGGTGATACCCAAATG | | |
| | | | CTACAGAGTGGAACACTCAGACCT | | |
| | | | GAGATTTGCAAAAACCAGATGT | | |
| | | | AAATATATGCATTCAAACATCAGG | | |
| | | | GCTTACTATGAGGTAGGTGGTATA | | |
| | | | TACATGTCACAAATAAAAATAC | | |
| | | | AGTTACAACTCAGGGTCACAAAAA | | |
| | | | ATGCATCTTCCAATGCATATTTTTA | | |
| | | | TTATGGTAAAATATACATAAA | | |
| | | | TATAATTCACCATTTTAACATTTAA | | |
| | | | TTCATATTAAATACGTACAAATCA | | |
| | | | GTGACATTTAGTACATTCACA | | |
| | | | GTGTTGTGCCACCATCACCACTATT | | |
| | | | TAGTTCCAGAACATTTGCATCATC | | |
| | | | AATACATTGTCTAGAGACAAG | | |
| | | | ACTATCCTGGGTAGGCAGAAACCA | | |
| | | | TAGATCTTTTGTGTTTACAGCTATG | | |
| | | | GAAACCAACTGTACCATAAAG | | |
| | | | ATAGTTCACTGAGTTTTAAACCCA | | |
| | | | AGCCACATCTTATTTTTCCAAGGTT | | |
| | | | TAATTTAGTGAGAGGGCAGCA | | |
| | | | TTAGTGTGGAGTGGCATGCTTTTGC | | |
| | | | CCTATCGTGGAATTTACACATCAG | | |
| | | | AATGTGCAGGATCCAAGTCTG | | |
| | | | AAAGTGTTGCCACCCGTCACACAA | | |
| | | | CATGGGCTTTGTTTGCTTATTCCAT | | |
| | | | GAAGCAGCAGCTATAGACCTT | | |
| | | | ACCATGGAAACATGAAGAGACCCT | | |
| | | | GCACCCCTTTCCTTAAGGATTGCTG | | |
| | | | CAAGAGTTACCTGTTGAGCAG | | |
| | | | GATTGACTGGTGATGTTTCATTCTG | | |
| | | | ACCTTGTCCCAAGCTCTCCATCTCT | | |
| | | | AGATCTGGGGACTGACTGTT | | |
| | | | GAGCTGATGGGGAAAGAAAAGCT | | |
| | | | CTCACACAAACCGGAAGCCAAATG | | |
| | | | TCCCCTATCTCTTGAATGATCAA | | |
| | | | GTCACTTTTGACAACATCCAGGTG | | |
| | | | AATATAAAAACTTAATAAAGCTGT | | |
| | | | GGAAAGGAACTCTTAATCTTCT | | |
| | | | TTTCTGCTACTTAGGTTAAATTCAC | | |
| | | | TAGATGTTGATTAGCAATCAAAAT | | |
| | | | TCGAATTGGGACATGTTCAAA | | |
| | | | TTCTTTCTTGTGGTAGTTGCCTATA | | |
| | | | CTGTCATCGCTGCTGTTGGTTGAGC | | |
| | | | ATTTGTGGTGTACCACGCTG | | |
| | | | TGTGGTCAAGGGTATTACATTCATG | | |
| | | | TTCTCATTTAATCCTCACAACAATC | | |
| | | | TGAAGAAGGTAGGTATTACA | | |
| | | | ATTCCCACTTCATAGAAACAGAAA | | |
| | | | CTGAGGTTCAGAGAGGTTAACTCA | | |
| | | | TTTGCCCAAATGGCTGAGCCAA | | |
| | | | AGCCTACCATGTACCTAACCTTTAT | | |
| | | | TTTCTTTCCCGAACATACCAGGCTG | | |
| | | | TCTCCTCATAACTTCCAAGC | | |
| | | | ATGCACTTAAAACTCCACATGAAT | | |
| | | | ACAAGGTTCATGGGACTTGGTATT | | |
| | | | CATAGAAAGGGAGGCAGAAAGC | | |
| | | | TGGTCTGTTCCTGATAGGCTTGTAA | | |
| | | | TTTAATATCATTCTGTTCATGTGCT | | |
| | | | TTGGATGGAAGCACATCTGG | | |
| | | | CATATGATGCTAATCAGTGGTTCC | | |
| | | | CATACCCCTGGCTTCCTAATTTTAA | | |
| | | | TGTTTGCTCACAGCATAGTAG | | |
| | | | ATTGACATCAAATAGTGGCCGATG | | |
| | | | ATGATGAAAATAAAGGTCAAATAA | | |
| | | | GTTGAGCCAATAACAGCCGCTT | | |
| | | | TTTTCCTTCTGTCTGCGTATACAAA | | |
| | | | GCACTGTCATGCACACAATCTATT | | |
| | | | CTCACCCTCACAACAACCCAT | | |
| | | | AAGGGTGTAAATAGTATTTCCATT | | |
| | | | TTACAAATGAGGATCACACAAACT | | |
| | | | ACTACATGGCAGAGCAGATACT | | |
| | | | CCAACTCATGTCTTCTGGTTGAAGC | | |
| | | | CTATTGCTTTTTCTTTTCTAAACAC | | |
| | | | TTTCCCTCAGCAAGTTGGAA | | |
| | | | TTAGACTTCACAAGTCTCCTTCAGA | | |
| | | | GAACACAAATCTTTTCTTATTCCAT | | |
| | | | TCCTGTTTGGTTGCCTACGT | | |
| | | | CCAATCTCCCCCTCCCCAGAGATG | | |
| | | | CCAAAAAAAAAATCCTTTAAGGTA | | |
| | | | TTTGGGAGCCAAACTCAACTTG | | |
| | | | TTAAAATCTCAAATTATGGAGACA | | |
| | | | ATCAGCAGACACAACCTAACCCCA | | |
| | | | ATTATTTTGGCAGGAAGGTTGG | | |
| | | | TTTAGAGGCAGATCCAGCAATCTG | | |
| | | | CTTTGGGCCACTCTGGGTGGGGTA | | |
| | | | GGTGAAATAAGATTGGTCACTG | | |
| | | | TTAACTAATTTTAATATTGGATTGG | | |
| | | | CCATTGGTTATCACTGATTACCATT | | |
| | | | CTCCCCTGGATTTTCACCCA | | |
| | | | GGACTCAAAACTTGGTTCTGCTAA | | |
| | | | CCCTGTTCCTTTATGAGGAACCTTT | | |
| | | | TAAAGATTCCTTTATAAGGTG | | |
| | | | GGAGTTTTTTTTCTATGAACCTATA | | |
| | | | GGGGAGAAAAAAGATCAGCAGAA | | |
| | | | GTCATTACTTTTTTTTTTTTTT | | |
| | | | TTTTTTTTTTTTGAGAGAGAGTCTC | | |
| | | | ACTCCATTGCCCAGGCTGGAGTGC | | |
| | | | AGTGGTGCTATCTCGGCTCAC | | |
| | | | TGCAACCTCCGCCTCCTGGGTTCA | | |
| | | | AGCAATTCTCCTGCCTCAGCCTCCC | | |
| | | | GAGTAGCTGGCATTGCAGGTC | | |
| | | | CCCACCACCACACCCGGCTAATTT | | |
| | | | TTGTATTTTTAGTAAAGACAGGGTT | | |
| | | | TCACCATGTTGGCCAGGCTGC | | |
| | | | TCTCCAACTCCCAATCTCAGGTGA | | |
| | | | TCCTATTGCCTCGGGCTCCCAAAG | | |
| | | | TGCTGGGATTACAGGAGTGAGC | | |
| | | | CACCATGCCTGGCCAGAAGTGGTT | | |
| | | | ACTTCTGTAGACAAAAGAATAATG | | |
| | | | CTACTTAATCAGGCTTTCTGTG | | |
| | | | TGACAACAAAGAGAAAGAAAATA | | |
| | | | AAGAAGTTTCAATTCATCCAATTCT | | |
| | | | TAATAAGAAATATGTAAATAAA | | |
| | | | ATTTTTTAAAATTACACTTCATTTT | | |
| | | | AATGTTGTATCAGTCAAGGTCCCT | | |
| | | | GCAAGAGATGGATGGTATGGT | | |
| | | | ACACTCAAACTGGGTAACACAGGA | | |
| | | | GAGTTTTCAGAAAGCAACTAAATC | | |
| | | | CAAAATACTATCAAGGAATCAA | | |
| | | | TATAAAAATTGTTAATATTTTTCTC | | |
| | | | ATACTAAATTTTCAAAATATTTTGT | | |
| | | | GTCTATTACATTTACAGCAC | | |
| | | | ATCTTAATTAGGACTAGCTGTGTGT | | |
| | | | TCACCTCACATGTGGCTTGTAGCTA | | |
| | | | CCATACTGGACAGCACATGT | | |
| | | | CCAAAAAAATACACGTAAAGTTAA | | |
| | | | AGTTTAAAAGACACAGGAACTAAG | | |
| | | | CCCTCATTGTCTTTCCCTTGGG | | |
| | | | AGGTAGTTTAAAGAGCTATAGATG | | |
| | | | CTGTAACATTCTTGCTATTATTTAT | | |
| | | | TATATATGACATTATTCCTAA | | |
| | | | AAAAGCTTTTGAGATCCTAGGTTG | | |
| | | | TATTCCTCAGGTTTTGTTGCCTTCC | | |
| | | | CATGAAGATGTGAAGGCAGGG | | |
| | | | ATGCCTGTTATTCAGTCCAAGATG | | |
| | | | CATGACAAGAGACCTTGGGAAAGT | | |
| | | | TTCATCTGGATTTAAAGATTAA | | |
| | | | TTCTTGATGCTTACATTCCATACTC | | |
| | | | AAAATGTAAATTTGAATATTAAAA | | |
| | | | TAAAGATGATTTTTTTTTTGG | | |
| | | | AGCTAGTCTTGCTCTGTTGCCCAGG | | |
| | | | CTGGAATGCAGTGGCATGATCATG | | |
| | | | GCTCACTGCAGCCTCGACCTC | | |
| | | | CCAACCTCAAGCAAGGCTACAGGT | | |
| | | | GTGCACCTAAGTAGCTAGGACTAC | | |
| | | | AGGTGTGCACCACCATGTCTAG | | |
| | | | CTATTTTTTTTTCTGTAGAGACAGG | | |
| | | | GTTTTCCTATGTTGTCCAGGCTGGT | | |
| | | | CTCGAACTCCTGCCCTCAAG | | |
| | | | CAATCCTCCTGCCTTGGCCTCCCAA | | |
| | | | AGTGTTGAGATTACAGGCGTAAGC | | |
| | | | CACTGCACCTGGCCAAGATGA | | |
| | | | ATATTTTAATAGCTCACAGAACAA | | |
| | | | AGTTTGCCACATAATGATAAAATT | | |
| | | | ACTATGAAAATATATTCCCTTT | | |
| | | | ATTGTCAGTTTAAAAGATGAACTG | | |
| | | | AGTTTCACCCAAACTGGTCTGGCC | | |
| | | | CCTCTCTGATTCAAATACCAAT | | |
| | | | AGTTGCTCTGATTCAAATTCCAACT | | |
| | | | CTTAGAACATGACAGCTGCTCATA | | |
| | | | ACTAGCTTTGCTTACTAACCA | | |
| | | | TGTTTCTTTCCATTTGTATTAGGTC | | |
| | | | CTTTACTTTTTATAACAGCCTCAAA | | |
| | | | GTTTCATGAATTGCTGCACT | | |
| | | | AAACATTGATTTTCATGTTTGTGAG | | |
| | | | TCTGCAAGCCAGCTGGGCAGCTCT | | |
| | | | ACTTCAGGTGGTAAGGGTGGA | | |
| | | | TCAGACCTATTCCATATACCTCTTG | | |
| | | | TTCTCCTTGTCCAGTGGTTTCTAGG | | |
| | | | GATATGTTCTCATGATGAAC | | |
| | | | CCCGCAGAGGCTCGTGAAAGTGAG | | |
| | | | AGGAAACTAGGATGCCTCTTAAGG | | |
| | | | TCTTGCTCAGGATGGGCTCTCC | | |
| | | | TGTCACTTCTGTCACAGGCTATTGT | | |
| | | | AAGTCATATGAGCAAGCTCAATAA | | |
| | | | AATATAAACAAGTCAGATAAA | | |
| | | | CAGTGGGAGGAATGGCAAAGTCAT | | |
| | | | ATGGCCAAGGCCATGACTGATTAA | | |
| | | | TTTTAACACAGGAAAAAAGTAA | | |
| | | | AGCATTAAATGCGATTATTTAATA | | |
| | | | TACAATGTCTTATTAACTGAAATAT | | |
| | | | AAAATGTGTTTACTGTAAAAT | | |
| | | | ATAATCTGTTTATCTCACCAAAGA | | |
| | | | AATATTATCTTTAAAAAATGTCATT | | |
| | | | ACTTCTAACACATCATCAGTC | | |
| | | | TGCAACTTCTTTCCATAGCCTTAAT | | |
| | | | CAGGATGCTGTGGCAGCTCCCACA | | |
| | | | TTAGCCTCGCATTCTAAACTG | | |
| | | | GTAGATGTCCTAGGAAACCATACA | | |
| | | | TCTATGTATTTTTCTTATTTTATAC | | |
| | | | GTTTAGGACAATGTATAGCTA | | |
| | | | ATTACCCAACTTTTTATTTGCATAC | | |
| | | | AAATCTAATACAACTGAACACAAT | | |
| | | | CAGTTTTATCACAGGTATAAT | | |
| | | | GGATTTTTCAATAGTGAGGAGGTG | | |
| | | | CCTCCATGAGCCTTCTCTTTAGAAA | | |
| | | | AGTGGCATTCAACACTCTTCA | | |
| | | | TTTGAAGTGAAGATTGCTATGTCTT | | |
| | | | TTGCATTGCTCTATTTTACATAAAT | | |
| | | | TAAGTTATAAATTGACACTA | | |
| | | | TAATCAACTGACACCATGATCAGT | | |
| | | | GATGATGATCACCCTCATCAGCAC | | |
| | | | TAGAGTTGACTTGTTTTTATAA | | |
| | | | CCCCTTTGCATGTATGTTGAATAGC | | |
| | | | AAAGTTCATCAGAGAACATGTATT | | |
| | | | AGTCAATGGTAAGTAAGATAC | | |
| | | | TCTCATCTAAGAAATAACATCACC | | |
| | | | TCTTCTAATGAAGTTCTAAGAAGA | | |
| | | | GAGGGAAGAAAAAGTCTTGGGA | | |
| | | | GCTAGTCAGGGAATAGTGTGTATT | | |
| | | | TGCAATTACCTAAACTGAACTCTA | | |
| | | | CCATTACTCCTAACCCAGTTCC | | |
| | | | TCCTCCTGTGTTTTACATGATTAAT | | |
| | | | GCCACCCCTGCCTCAATGAACCAA | | |
| | | | GATCAGCTCCATCACTGGGAC | | |
| | | | CTCCCCATTCTGCCTGTGCAATATT | | |
| | | | TTTCTTTTTTATTTCTCCTTCTAATA | | |
| | | | TTACTGTTATTGCTCCAGT | | |
| | | | AAAGAGCTGTAATATATTTTACCT | | |
| | | | CGACTGATACCAGGAATGGTGGTG | | |
| | | | TTGCTTCCAATCTGTTGCTGCT | | |
| | | | AGATTAATCTTTGCAAAGCACAGG | | |
| | | | CTTAATTTCATTGCTGCTCAACTAA | | |
| | | | AACCACTGGTGGCTTTCCATT | | |
| | | | GCCTACAAAATAAAGTCAACCTCC | | |
| | | | CCATCAGACATTCAAGGCTTTCAA | | |
| | | | TGATCCATGGCCGCCAGCTCTC | | |
| | | | TCCAGGCTCATATCCCACTCCACTC | | |
| | | | CTCTGATGTTTCCTACACTACACTA | | |
| | | | CACTATACTACACTACAGCC | | |
| | | | AGGTAGAATGACTGTTCACCCAAC | | |
| | | | ACCACTCAGGTTGTCTTCTCAACTT | | |
| | | | GGAATACTCTTGCACCTTCAA | | |
| | | | AGCTCATTTCAAATGCCCCTTCATT | | |
| | | | TGTGAAGCCTTCTCCAAATTTCCAA | | |
| | | | GTCAGAATGTCTCTTCCTTG | | |
| | | | TGCTACCACAACCCTTTAACTGAG | | |
| | | | CCTCCATTAGTGCACTGAGACCAT | | |
| | | | TCTGTTCAGTGTCTGGGTGAAG | | |
| | | | CTTCCTGGTGAAAAATATGTTACCT | | |
| | | | ATTTCTTTCTGAAAAGTTGGATTCA | | |
| | | | GGGATATTATCACGGACCTA | | |
| | | | AGGTAATACTTCTAGCCAACCTCC | | |
| | | | CTGTCCACTGCCAGGCCGACTACA | | |
| | | | AACCCTTCTGTTGCTGGCGAGC | | |
| | | | TGGTCCGCACCACTAGTTCTGCTTC | | |
| | | | ACTCTATTTATCTCTTGATGTAACC | | |
| | | | ATCTTCTTTCTCCAGGTTTT | | |
| | | | AAGAACCAGCCCAACTCCTGGTTC | | |
| | | | CCTGATGAAGCTTTTATTCCCCTAG | | |
| | | | CCACATGGAACTTTTCCTTTT | | |
| | | | TGGAACATGCCTTTAGTTTCTGTGT | | |
| | | | AGTTTGCCATGCAGCACTTCATTGT | | |
| | | | ACACATTATTAAAACAGAAT | | |
| | | | TTTAAGGATTAGAATGAACCTTAA | | |
| | | | AAGATCATGCATCTCAAAATTTAA | | |
| | | | TGTACATACAAATTACCCAGGG | | |
| | | | ATTTTGTTGAAATAAAAATTATTTA | | |
| | | | ATTTTAATTAATATAAATAATTCAG | | |
| | | | TAGGTCTGGGGTGAGGCCTG | | |
| | | | AGGTTTTACATTTCCAACAAGCTG | | |
| | | | CCAGGTAAAGCCAATACATCTGTC | | |
| | | | CAGGAATCACACTTTGCGTATC | | |
| | | | AAAGGTCTAGATGACATTATCATT | | |
| | | | CCAAAGAGTTTCTTTTACAGGCTCT | | |
| | | | CACATCAGTGTTCATCCACTA | | |
| | | | CCTGACTACTGTCATTCACAGGCA | | |
| | | | TTCTGTTCCACAGCAGGCCAGCTA | | |
| | | | ACGTGGTATTTACAAAGCTCAC | | |
| | | | TCCTCTTATACAACAATCCAAGTGT | | |
| | | | TTCTTTTGTCAGTTGTCTGTGCCCC | | |
| | | | AGGAGATCCCTCTCTGCCTT | | |
| | | | GCCTTGCCCTCTGCCTTTGGAGACC | | |
| | | | AGCACCTCATACTCAGTGAAGGCC | | |
| | | | TGGAGTGCTTAAGAGGGATTT | | |
| | | | CTTCCAGCTCTCTTGCCCTGGTCTT | | |
| | | | CAGTGTATTAGATGTATTACCTCCA | | |
| | | | TGCTCTCAGTAGAGGCCCAT | | |
| | | | AGGAAAGAGTAGGTAGGTTATGCC | | |
| | | | AGCTCACACGCATCCTTTAAAAAT | | |
| | | | GGTTTAGAAGTTTAGCTGGTTT | | |
| | | | CTTATTACTCCTGTCTATGGATGTT | | |
| | | | TCCTTCTGTCACTCTACTAGGGATG | | |
| | | | AAACAGCTAATCATGTTCAA | | |
| | | | TAGTTACATTTAGATTGGTTTTTAA | | |
| | | | AAACTATGATTGTATTAGTTCGTTT | | |
| | | | CCATGCTGCTGATAAAGACA | | |
| | | | TATCTGAGACTGGAAACAAAAAGG | | |
| | | | GTTTAATTGGACTTACAGTTCCACA | | |
| | | | TGGCTGGGGAGGCCTCAAAAT | | |
| | | | CACGTGGGAGGCAAAAGGTACTTC | | |
| | | | TTACGTGGTGGCATCAAGAGCAAA | | |
| | | | ATGAGGAAGAAGCAAAAGCAGA | | |
| | | | AACTCTTCATAAACCCACCAGATC | | |
| | | | TTGTGGGACTTATTATCACGAGAA | | |
| | | | TAGCACAGAAAAGACTGGCCTC | | |
| | | | CATGATTCAATTACCTCCCACTGC | | |
| | | | GTCCCTCCCACAACATGTGGGAAT | | |
| | | | TCTGGGAGATACAATTCAAGTT | | |
| | | | GAGATTTGGGTGGGGACACAGCCA | | |
| | | | AACCATATCATTCCTCCCTGGGCTC | | |
| | | | CTCCAAATTTCATAATCCTCA | | |
| | | | CATTTCAAAACCAATCATTCCTTCC | | |
| | | | CAACAGTTCCCCAAAGTCTTAACT | | |
| | | | CATTTCAGCATTAACCCAAAA | | |
| | | | GTCCACAGTCCAAAGTCTCATCTG | | |
| | | | AGACAAGGCAAGTCCCTTCCACTT | | |
| | | | ACAAGCCTGTAAAAGCAAGCTA | | |
| | | | GTTACCTCCTAGATACAATGGGGG | | |
| | | | GTACAGGTATTGGGTAAATACAGC | | |
| | | | TGTTCCAAATGAGAGAAATTGG | | |
| | | | CCAAAACAAAGGGGTTACAGGGTC | | |
| | | | CATGCAAGTCTGAAATCCAGTGGG | | |
| | | | GCAGTCAAATTTTAAAGCTCCA | | |
| | | | TAATGATCTCCTTTGACTCCATGTC | | |
| | | | TCACATTCAGGTCATGCTGATCCA | | |
| | | | AGAGATAGGTTCCCATGGTCT | | |
| | | | TGTGCACCTCCGCCCCTGTGGCTTT | | |
| | | | GCAGAGTACAGCCTCCCTCCTGGC | | |
| | | | TGCTTTCTCAGGCTGATGTTG | | |
| | | | AGTGTCTGTAGCTTTTCCAGGCAC | | |
| | | | AAGATGCAAGTTGGTGGTTGATCT | | |
| | | | ACCATTCTGGGGTCTACCATTC | | |
| | | | TGGGGTCTACCGTTCTGGGACTGT | | |
| | | | GGCCTTCTTCTCACAGCTCCACTAG | | |
| | | | GCAGTGCCCCAACAGGGACTC | | |
| | | | TGTGTGGGGGCTCTGCCCCACATTT | | |
| | | | CCCTTCCACACTGCCCTAGGAGAG | | |
| | | | GTTCCCCATGAGGGCTCTGCC | | |
| | | | CCTGCAGCAAACTTTTGCCTGGAC | | |
| | | | ATCCAGGTGTTTCCATATATATTCT | | |
| | | | GAAATCTAGGCACAGGTTCCC | | |
| | | | AAATCTCAATTCTTGACATCTCTGC | | |
| | | | ACCCACAGGCTCAACATCACATGG | | |
| | | | AAGCTGCCAATGCTTGGGGCC | | |
| | | | TCTACCCTCTGAAGCCACAGCCCA | | |
| | | | AGCTCTATGTTGGCTCCTTTCAGCC | | |
| | | | ATGGCTGGAGCAGCTGGGACA | | |
| | | | CAGGGCACCAAGTCCCTAGGCTGC | | |
| | | | ACACAGCACAGAGACCCTGGGCCC | | |
| | | | AGCCCACAAAACCACTTTTTCC | | |
| | | | TCCTGGGCCTCTGGGCCTGTGATG | | |
| | | | GGAGGGGCTGCCATGAAGGTCTCT | | |
| | | | GACATGACCTGCAGACATTTTC | | |
| | | | CCCATGGTCTTGGGGATTAACATT | | |
| | | | AGGCTCCTTGCTGCTTATGCAAATT | | |
| | | | TCTGCAGCCAGCTTGAATTTC | | |
| | | | TCCTTAAAAAAAATGGGTTTTTCTT | | |
| | | | TTCTACTGCATCATCAGGCTGCAG | | |
| | | | ATTTTCCACATTTATGCTCTT | | |
| | | | GTTTCCCTTTTAAAACAGAATGTTT | | |
| | | | TTAACAGCACCCAAGTCACCTTTT | | |
| | | | GAATGCTTTGCTGCTTAGAAA | | |
| | | | TTTATTCCACCAGATACCCTAAGTC | | |
| | | | ATCTCTCTCAAGCTCTAAGTTCCAC | | |
| | | | AAATCTCTAGGGCAAGGGTG | | |
| | | | AAATGCTGCCAGTCTCCTTGCTAA | | |
| | | | AACATAACAAGGGTCACCTTTACT | | |
| | | | TCAGTTCCCAACAAGGTCTTCA | | |
| | | | TCTCCATCTGAGACCACCTCAGCC | | |
| | | | TGGACCTTATTGTTCATATCACTAT | | |
| | | | CAGTATTTTTGTCAATGCCAT | | |
| | | | TCACAGTCTCTAGGAGGTTCCAAA | | |
| | | | CTTTCCTACATTTTCCTATCTTCTTC | | |
| | | | TGAGCCCTCCAGATTATTTC | | |
| | | | AACACCCAGTTCCAAAGTTGCTTC | | |
| | | | CACATTTTCGGGTATCTTTTCAGCA | | |
| | | | ATGCCCCACTCTACTGGTACT | | |
| | | | ATTAGTCCATTTTCATGCTGCTGAT | | |
| | | | AAAGACATACCTGAGACTGGGAAC | | |
| | | | AAAAAGAGGTTTAATTGGACT | | |
| | | | TATAGTTCCACCTGGCTGGGGAGG | | |
| | | | CCTCAGAATCATGGCAGGAGGTCA | | |
| | | | AAGGCATTTCTTACACGGCAGC | | |
| | | | AGCAAGAGAAAAATGAAGAAGCA | | |
| | | | CCAAAAGCAGAAACCCCTGATAA | | |
| | | | AACCATCAGATCTCGTGAGACTTA | | |
| | | | TTCACTATCACAAGAATAGCATGG | | |
| | | | GAAAGACCAGCCCCCTTGATTCAA | | |
| | | | TTACCTCCCCCTGGGTCCTGTG | | |
| | | | GGAATTCTGGAAGGTACAATTCAA | | |
| | | | GTTGAGATTTGGGTGGGGACACAG | | |
| | | | CCAAACCATATCAATGATTTTG | | |
| | | | TACTTTAACCAGCTGAATGGAAGT | | |
| | | | ACAATCTCTTGCTATATGACACAA | | |
| | | | TAATTATTTGCAAAATGAGTAA | | |
| | | | ACATATCATAAGGAAATTATTTTT | | |
| | | | ACAAGGTTTGAAACCTGAAATGCA | | |
| | | | GTCTATTATCATACATAACTAA | | |
| | | | AAATAGAGCCTCAATAAACAGATT | | |
| | | | CCCAGTTTTGAAAATGCAACATTT | | |
| | | | GTACTCCACATTGTCAGTTTTC | | |
| | | | TTAGGTATATTTATAAATACTCCTA | | |
| | | | TAAAAATGTAAAGAAACACATAAT | | |
| | | | GTAGATTGCTAATTTTATAAT | | |
| | | | AACACAAGTTGATTTTGACATCCA | | |
| | | | ACTTATTAATTATGAAATGACTTTT | | |
| | | | GGCCTAGTAACAATGAAAATG | | |
| | | | GGGGCAAATACAGATAAATGGTAA | | |
| | | | TTCTTAGAATGAACTACTCACCAC | | |
| | | | CAATTCTAAGTTTTTCTTGATG | | |
| | | | GTAAATCATAATGTTCCCTTTCTCC | | |
| | | | TCGGTTCTGCAATCTATAGGCATA | | |
| | | | CCATAATTGTAATCAATAGCT | | |
| | | | TAAAAATATGTCTCTCTGTCCTATT | | |
| | | | CTGTATCTGTATCTCTTGGATTTTT | | |
| | | | ACCTTTGCAATAGTCAACTG | | |
| | | | AACCATCTTCTTGGAGTACTCATG | | |
| | | | AAGATGGAAGTCTACATGGAGAAT | | |
| | | | ACAGGATGAATCCACTCTGTCT | | |
| | | | CCTGCAGTGAAGTCTGTTTGAAGG | | |
| | | | ATGTATTTGGCTGTCTTCTGGACAG | | |
| | | | GCCATTCTAATAACAGAAACA | | |
| | | | AACAAGTTATTTTAAAACTTATTG | | |
| | | | GAATATTCAAATATTAACCAAAGT | | |
| | | | AGAAAAATATAATACACATCCA | | |
| | | | TGTGCCCATCACAGAACTTCACTG | | |
| | | | ATTATCATCATTTAGCCAGTCTTGA | | |
| | | | AGAAGCAAGTGCTAATTACAA | | |
| | | | TCACAAATGAAACAAGATTCAGAC | | |
| | | | TTCATGAAGAGCACTGCGCTATAA | | |
| | | | TAAAAGAAGAAATGAGCACATA | | |
| | | | CATTCTTTTACTGACAGTCAAATGG | | |
| | | | TGAAGGTGGGCAGAATCATTATGT | | |
| | | | GATGCAACATGGCAAAAGTAT | | |
| | | | ACAGACAGTGCATCCAGAGGAAG | | |
| | | | GCACCTTGCTGAATGACTAGAATG | | |
| | | | CAAGTAGGAGACATTTTGCAGGC | | |
| | | | CCCCTTCATCCTGCAGGGAGAACC | | |
| | | | AGAACCACAGCAGCTCTATTTGCC | | |
| | | | TATTCCTCTTTAAATTACAAAG | | |
| | | | TTAAAATTTGGGAGTAGTAGAAAA | | |
| | | | TCAATTGGTTATCTTATAGAGTCTC | | |
| | | | CTAGAATATTTCATTGGCATT | | |
| | | | GAGAAGGTGGAAAATGCAAATTAT | | |
| | | | ATACTTTAAAATGTAATTTTTGCTT | | |
| | | | TTCACATATGCTTAAAGCCTA | | |
| | | | AAACCTCTTAATAAACTTCTTCTGA | | |
| | | | AATATA (SEQ ID NO: 42) | | |
| | | NM_001320916.1 | CCTGTTGCTCTTTGCTCTAATGAGC | NP_001307845.1 | MGREELFLTFSFSSGFQ |
| | | | CTTGAGAAAGGATTGCTGGTCATG | | ESNVKTFCSKNILAILGF |
| | | | GGACCAGAGGCTTTATGGGGA | | SSILAVIAL |
| | | | GGGAAGAACTGTTCTTCACTTTCA | | LAVGLTQNKALPENVK |
| | | | GTTTTTCGAGCGGGTTTCAAGAGT | | YGIVLDAGSSHTSLYTY |
| | | | CTAACGTGAAGACATTTTGCTC | | KWPAEKENDTGVVHQV |
| | | | CAAGAATATCCTAGCCATCCTTGG | | EECRVKGPG |
| | | | CTTCTCCTCTATCATAGCTGTGATA | | ISKFVQKVNEIGIYLTDC |
| | | | GCTTTGCTTGCTGTGGGGTTG | | MERAREVIPRSQHQETP |
| | | | ACCCAGAACAAAGCATTGCCAGAA | | VYLGATAGMRLLRMES |
| | | | AACGTTAAGTATGGGATTCTGCTG | | EELADRV |
| | | | GATGCGGGTTCTTCTCACACAA | | LDVVERSLSNYPFDFQG |
| | | | GTTTATACATCTATAAGTGGCCAG | | ARIITGQEEGAYGWITIN |
| | | | CAGAAAAGGAGAATGACACAGGC | | YLLGKFSQKTRWFSIVP |
| | | | GTGGTGCATCAAGTAGAAGAATG | | YETNNQ |
| | | | CAGGGTTAAAGGTCCTGGAATCTC | | ETFGALDLGGASTQVTF |
| | | | AAAATTTGTTCAGAAAGTAAATGA | | VPQNQTIESPDNALQFR |
| | | | AATAGGCATTTACCTGACTGAT | | LYGKDYNVYTHSFLCY |
| | | | TGCATGGAAAGAGCTAGGGAAGTG | | GKDQALWQ |
| | | | ATTCCAAGGTCCCAGCACCAAGAG | | KLAKDIQVASNEILRDP |
| | | | ACACCCGTTTACCTGGCAGCCA | | CFHPGYKKVVNVSDLY |
| | | | CGGCAGGCATGCGGTTGCTCAGGA | | KTPCTKRFEMTLPFQQF |
| | | | TGGAAAGTGAAGAGTTGGCAGACA | | EIQGIGNY |
| | | | GGGTTCTGGATGTGGTGGAGAG | | QQCHQSILELFNTSYCP |
| | | | GAGCCTCAGCAACTACCCCTTTGA | | YSQCAFNGIFLPPLQGD |
| | | | CTTCCAGGGTGCCAGGATCATTAC | | FGAFSAFYFVMKFLNLT |
| | | | TGGCCAAGAGGAAGGTGCCTAT | | SEKVSQE |
| | | | GGCTGGATTACTATCAACTATCTG | | KVTEMMKKFCAQPWE |
| | | | CTGGGCAAATTCAGTCAGAAAACA | | EIKTSYAGVKEKYLSEY |
| | | | AGGTGGTTCAGCATAGTCCCAT | | CFSGTYILSLLLQGYHFT |
| | | | ATGAAACCAATAATCAGGAAACCT | | ADSWEHIH |
| | | | TTGGAGCTTTGGACCTTGGGGGAG | | FIGKSTEPSSWSTHEDGS |
| | | | CCTCTACACAAGTCACTTTTGT | | LHGEYRMNPLCLLQ |
| | | | ACCCCAAAACCAGACTATCGAGTC | | (SEQ ID NO: 50) |
| | | | CCCAGATAATGCTCTGCAATTTCG | | |
| | | | CCTCTATGGCAAGGACTACAAT | | |
| | | | GTCTACACACATAGCTTCTTGTGCT | | |
| | | | ATGGGAAGGATCAGCCACTCTGGC | | |
| | | | AGAAACTGGCCAAGGACATTC | | |
| | | | AGGTTGCAAGTAATGAAATTCTCA | | |
| | | | GGGACCCATGCTTTCATCCTGGAT | | |
| | | | ATAAGAAGGTAGTGAACGTAAG | | |
| | | | TGACCTTTACAAGACCCCCTGCAC | | |
| | | | CAAGAGATTTGAGATGACTCTTCC | | |
| | | | ATTCCAGCAGTTTGAAATCCAG | | |
| | | | GGTATTGGAAACTATCAACAATGC | | |
| | | | CATCAAAGCATCCTGGAGCTCTTC | | |
| | | | AACACCACTTACTGCCCTTACT | | |
| | | | CCCAGTGTGCCTTCAATGGGATTTT | | |
| | | | CTTGCCACCACTCCAGGGGGATTT | | |
| | | | TGGGGCATTTTCAGCTTTTTA | | |
| | | | CTTTGTGATGAAGTTTTTAAACTTG | | |
| | | | ACATCAGAGAAAGTCTCTCAGGAA | | |
| | | | AAGGTGACTGAGATCATGAAA | | |
| | | | AAGTTCTGTGCTCAGCCTTGGGAG | | |
| | | | GAGATAAAAACATCTTACGCTGGA | | |
| | | | GTAAAGGAGAAGTACCTGAGTG | | |
| | | | AATACTGCTTTTCTGGTACCTACAT | | |
| | | | TCTCTCCCTCCTTCTGCAAGGCTAT | | |
| | | | CATTTCACAGCTGATTCCTG | | |
| | | | GGAGCACATCCATTTCATTGGCAA | | |
| | | | GTCAACTGAACCATCTTCTTGGAG | | |
| | | | TACTCATGAAGATGGAAGTCTA | | |
| | | | CATGGAGAATACAGGATGAATCCA | | |
| | | | CTCTGTCTCCTGCAGTGAAGTCTGT | | |
| | | | TTGAAGGATGTATTTGGCTGT | | |
| | | | CTTCTGGACAGGCCATTCTAATAA | | |
| | | | CAGAAACAAACAAGTTATTTTAAA | | |
| | | | ACTTATTGGAATATTCAAATAT | | |
| | | | TAACCAAAGTAGAAAAATATAATA | | |
| | | | CACATCCATGTGCCCATCACAGAA | | |
| | | | CTTCACTGATTATCATCATTTA | | |
| | | | GCCAGTCTTGAAGAAGCAAGTGCT | | |
| | | | AATTACAATCACAAATGAAACAAG | | |
| | | | ATTCAGACTTCATGAAGAGCAC | | |
| | | | TGCGCTATAATAAAAGAAGAAATG | | |
| | | | AGCACATACATTCTTTTACTGACA | | |
| | | | GTCAAATGGTGAAGGTGGGCAG | | |
| | | | AATCATTATGTGATGCAACATGGC | | |
| | | | AAAAGTATACAGACAGTGCATCCA | | |
| | | | GAGGAAGGCACCTTGCTGAATG | | |
| | | | ACTAGAATGGAAGTAGGAGACATT | | |
| | | | TTGCAGGCCCCCTTCATCCTGCAG | | |
| | | | GGAGAACCAGAACCACAGCAGC | | |
| | | | TCTATTTGCCTATTCCTCTTTAAAT | | |
| | | | TACAAAGTTAAAATTTGGGAGTAG | | |
| | | | TAGAAAATCAATTGGTTATCT | | |
| | | | TATAGAGTCTCCTAGAATATTTCAT | | |
| | | | TGGCATTGAGAAGGTGGAAAATGC | | |
| | | | AAATTATATACTTTAAAATGT | | |
| | | | AATTTTTGCTTTTCACATATGCTTA | | |
| | | | AAGCCTAAAACCTCTTAATAAACT | | |
| | | | TCTTCTGAAATATAAAAAAAA | | |
| | | | A (SEQ ID NO: 49) | | |
| CLIC1 | Chloride | NM_001287593.1 | CCAAGTAGCTGGGATTACAGGTGC | NP_001274522.1 | MAEEQPQVELFVKAGS |
| | Intra- | | CCACCACCCCGCCTGGCAAATTTT | | DGAKIGNCPFSQRLFMV |
| | cellular | | TGTATTTTTAGTAGAGACAGGG | | LWLKGVTFNVT |
| | Channel 1 | | TTTCACCATGTTGGCCAGTCTGGTC | | TVDTKRRTETVQKLCPG |
| | | | TTGACTCCCTGACCTCAGGTGATC | | GQLPFLLYGTEVHTDTN |
| | | | CACCCCCCTTGGCCTCCTAAA | | KIEEFLEAVLCPPRYPKL |
| | | | GTGTTGGGATTACAGGCGTGAGCC | | AALNPE |
| | | | ACCTCACCCGGCCCCTAACTCTATT | | SNTAGLDIFAKFSAYIK |
| | | | TCCTATGCCCAATCCCAAGTG | | NSNPALNDNLEKGLLK |
| | | | TAGGCCACAAGGACTGCAAGTCCT | | ALKVLDNYLTSPLPEEV |
| | | | AGTGCTGAGCTGGGCCCGGAGACA | | DETSAEDE |
| | | | GTAGACTGCGGGGGGCACAGGA | | GVSQRKFLDGNELTLA |
| | | | CCTACTGAGACACCAGTCTGGGCA | | DCNLLPKLHIVQVVCKK |
| | | | GCTCAGGGAGTGCTGGCGTCACCC | | YRGFTIPEAFRGVHRYL |
| | | | CTTCCCTAATCCCAGGCTGCAT | | SNAYAREE |
| | | | GGCTAACGGTTCCTATCTGCAGTC | | FASTCPDDEEIELAYEQ |
| | | | CCAGCCTTCCACTTCCGAGTTCTTC | | VAKALK (SEQ ID NO: |
| | | | TCTCAGACCACAGTCCCAGCA | | 52) |
| | | | ACCCAGAATTTGGATTGGAGTCTG | | |
| | | | GAAGAAATGCAGAATGATTAAACG | | |
| | | | ACCACCTTTCCATTTGAAGTCC | | |
| | | | CCATCCCTGAATCTTCACGGGTGT | | |
| | | | CCCCAAGCTCCCCTCCCAGTTCCC | | |
| | | | CCAGGGACGGCCACTTCCTGGT | | |
| | | | CCCCGACGCAACCATGGCTGAAGA | | |
| | | | ACAACCGCAGGTCGAATTGTTCGT | | |
| | | | GAAGGCTGGCAGTGATGGGGCC | | |
| | | | AAGATTGGGAACTGCCCATTCTCC | | |
| | | | CAGAGACTGTTCATGGTACTGTGG | | |
| | | | CTCAAGGGAGTCACCTTCAATG | | |
| | | | TTACCACCGTTGACACCAAAAGGC | | |
| | | | GGACCGAGACAGTGCAGAAGCTGT | | |
| | | | GCCCAGGGGGGCAGCTCCCATT | | |
| | | | CCTGCTGTATGGCACTGAAGTGCA | | |
| | | | CACAGACACCAACAAGATTGAGG | | |
| | | | AATTTCTGGAGGCAGTGCTGTGC | | |
| | | | CCTCCCAGGTACCCCAAGCTGGCA | | |
| | | | GCTCTGAACCCTGACTCCAACACA | | |
| | | | GCTGGGCTGGACATATTTGCCA | | |
| | | | AATTTTCTGCCTACATCAAGAATTC | | |
| | | | AAACCCAGCACTCAATGACAATCT | | |
| | | | GGAGAAGGGACTCCTGAAAGC | | |
| | | | CCTGAAGGTTTTAGACAATTACTT | | |
| | | | AACATCCCCCCTCCCAGAAGAAGT | | |
| | | | GGATGAAACCAGTGCTGAAGAT | | |
| | | | GAAGGTGTCTCTCAGAGGAAGTTT | | |
| | | | TTGGATGGCAACGAGCTCACCCTG | | |
| | | | GCTGACTGCAACCTGTTGCCAA | | |
| | | | AGTTACACATAGTACAGGTGGTGT | | |
| | | | GTAAGAAGTACCGGGGATTCACCA | | |
| | | | TCCCCGAGCCCTTCCGGGGAGT | | |
| | | | GCATCGGTACTTGAGCAATGCCTA | | |
| | | | CGCCCGGGAAGAATTCGCTTCCAC | | |
| | | | CTGTCCAGATGATGAGGAGATC | | |
| | | | GAGCTCGCCTATGAGCAAGTGGCA | | |
| | | | AAGGCCCTCAAATAAGCCCCTCCT | | |
| | | | GGGACTCCCTCAACCCCCTCCA | | |
| | | | TTTTCTCCACAAAGGCCCTGGTGGT | | |
| | | | TTCCACATTGCTACCCAATGGACA | | |
| | | | CACTCCAAAATGGCCAGTGGG | | |
| | | | CAGGGAATCCTGGACCACTTGTTC | | |
| | | | CGGGATGGTGTGGTGGAAGAGGG | | |
| | | | GATGAGGGAAAGAAATGGGGGGC | | |
| | | | CTGGGTCAGATTTTTATTGTGGGGT | | |
| | | | GGGATGAGTAGGACAACATATTTC | | |
| | | | AGTAATAAAATACAGAATAAA | | |
| | | | AATCAAGTGTTTTTACGCAAAAAA | | |
| | | | AAAAAAAAAA (SEQ ID NO: 51) | | |
| | | NM_001288.4 | GTTCAGGGGGGGGCCGGTCGGTGA | NP_001279.2 | MAEEQPQVELFVKAGS |
| | | | GTCAGCGGCTCTCTGATCCAGCCC | | DGAKIGNCPFSQRLFMV |
| | | | GGGAGAGGACCGAGCTGGAGGA | | LWLKGVTFNVT |
| | | | GCTGGGTGTGGGGTGCGTTGGGCT | | TVDTKRRTETVQKLCPG |
| | | | GGTGGGGAGGCCTAGTTTGGGTGC | | GQLPFLLYGTEVHTDTN |
| | | | AAGTAGGTCTGATTGAGCTTGT | | KIEEFLEAVLCPPRYPKL |
| | | | GTTGTGCTGAAGGGACAGCCCTGG | | AALNPE |
| | | | GTCTAGGGGAGAGAGTCCCTGAGT | | SNTAGLDIFAKFSAYIK |
| | | | GTGAGACCCGCCTTCCCCGGTC | | NSNPALNDNLEKGLLK |
| | | | CCAGCCCCTCCCAGTTCCCCCAGG | | ALKVLDNYLTSPLPEEV |
| | | | GACGGCCACTTCCTGGTCCCCGAC | | DETSAEDE |
| | | | GCAACCATGGCTGAAGAACAAC | | GVSQRKFLDGNELTLA |
| | | | CGCAGGTCGAATTGTTCGTGAAGG | | DCNLLPKLHIVQVVCKK |
| | | | CTGGCAGTGATGGGGCCAAGATTG | | YRGFTIPEAFRGVHRYL |
| | | | GGAACTGCCCATTCTCCCAGAG | | SNAYAREE |
| | | | ACTGTTCATGGTACTGTGGCTCAA | | FASTCPDDEEIELAYEQ |
| | | | GGGAGTCACCTTCAATGTTACCAC | | VAKALK (SEQ ID NO: |
| | | | CGTTGACACCAAAAGGCGGACC | | 54) |
| | | | GAGACAGTGCAGAAGCTGTGCCCA | | |
| | | | GGGGGGCAGCTCCCATTCCTGCTG | | |
| | | | TATGGCACTGAACTGCACACAG | | |
| | | | ACACCAACAAGATTGAGGAATTTC | | |
| | | | TGGAGGCAGTGCTGTGCCCTCCCA | | |
| | | | GGTACCCCAAGCTGGCAGCTCT | | |
| | | | GAACCCTGAGTCCAACACAGCTGG | | |
| | | | GCTGGACATATTTGCCAAATTTTCT | | |
| | | | GCCTACATCAAGAATTCAAAC | | |
| | | | CCAGCACTCAATGACAATCTGGAG | | |
| | | | AAGGGACTCCTGAAAGCCCTGAAG | | |
| | | | GTTTTAGACAATTACTTAACAT | | |
| | | | CCCCCCTCCCAGAAGAAGTGGATG | | |
| | | | AAACCAGTGCTGAAGATGAAGGTG | | |
| | | | TCTCTCAGAGGAAGTTTTTGGA | | |
| | | | TGGCAACGAGCTCACCCTGGCTGA | | |
| | | | CTGCAACCTGTTGCCAAAGTTACA | | |
| | | | CATAGTACAGGTGGTGTGTAAG | | |
| | | | AAGTACCGGGGATTCACCATCCCC | | |
| | | | GAGGCCTTCCGGGGAGTGCATCGG | | |
| | | | TACTTGAGCAATGCCTACGCCC | | |
| | | | GGGAAGAATTCGCTTCCACCTGTC | | |
| | | | CAGATGATGAGGAGATCGAGCTCG | | |
| | | | CCTATGAGCAAGTGGCAAAGGC | | |
| | | | CCTCAAATAAGCCCCTCCTGGGAC | | |
| | | | TCCCTCAACCCCCTCCATTTTCTCC | | |
| | | | ACAAAGGCCCTGGTGGTTTCC | | |
| | | | ACATTGCTACCCAATGGACACACT | | |
| | | | CCAAAATGGCCAGTGGGCAGGGA | | |
| | | | ATCCTGGAGCACTTGTTCCGGGA | | |
| | | | TGGTGTGGTGGAAGAGGGGATGAG | | |
| | | | GGAAAGAAATGGGGGGCCTGGGT | | |
| | | | CAGATTTTTATTGTGGGGTGGGA | | |
| | | | TGAGTAGGACAACATATTTCAGTA | | |
| | | | ATAAAATACAGAATAAAAATCAAG | | |
| | | | TGTTTTTACGCAAAAAAAAAAA | | |
| | | | AAAAA (SEQ ID NO: 53) | | |
| | | NM_001287594.1 | GGTGAGTCAGCGGCTCTCTGATCC | NP_001274523.1 | MAEEQPQVELFVKAGS |
| | | | AGCCCGGGAGAGGACCGAGCTGG | | DGAKIGNCPFSQRLFMV |
| | | | AGGAGCTGGGTGTGGGCCCCTCC | | LWLKGVTFNVT |
| | | | CAGTTCCCCCAGGGACGGCCACTT | | TVDTKRRTETVQKLCPG |
| | | | CCTGGTCCCCGACGCAACCATGGC | | GQLPFLLYGTEVHTDTN |
| | | | TGAAGAACAACCGCAGGTCGAA | | KIEFLEAVLCPPRYPKL |
| | | | TTGTTCGTGAAGGCTGGCAGTGAT | | AALNPE |
| | | | GGGGCCAAGATTGGGAACTGCCCA | | SNTAGLDIFAKFSAYIK |
| | | | TTCTCCCAGAGACTGTTCATGG | | NSNPALNDNLEKGLLE |
| | | | TACTGTGGCTCAAGGGAGTCACCT | | ALKVLDNYLTSPLPEEV |
| | | | TCAATGTTACCACCGTTGACACCA | | DETSAEDE |
| | | | AAAGGCGGACCGAGACAGTGCA | | GVSQRKFLDGNELTLA |
| | | | GAAGCTGTGCCCAGGGGGGCAGCT | | DCNLLPKLHIVQVVCKK |
| | | | CCCATTCCTGCTGTATGGCACTGA | | YRGFTIPEAFRGVHRYL |
| | | | AGTGCACACAGACACCAACAAG | | SNAYAREE |
| | | | ATTGAGGAATTTCTGGAGGCAGTG | | FASTCPDDEGIELAYEQ |
| | | | CTGTGCCCTCCCAGGTACCCCAAG | | VAKALK (SEQ ID NO: |
| | | | CTGGCAGCTCTGAACCCTGAGT | | 56) |
| | | | CCAACACAGCTGGGCTGGACATAT | | |
| | | | TTGCCAAATTTTCTGCCTACATCAA | | |
| | | | GAATTCAAACCCAGCACTCAA | | |
| | | | TGACAATCTGGAGAAGGGACTCCT | | |
| | | | GAAAGCCCTGAAGGTTTTAGACAA | | |
| | | | TTACTTAACATCCCCCCTCCCA | | |
| | | | GAAGAAGTGGATGAAACCAGTGCT | | |
| | | | GAAGATGAAGGTGTCTCTCAGAGG | | |
| | | | AAGTTTTTGGATGGCAACGAGC | | |
| | | | TCACCCTGGCTGACTGCAACCTGT | | |
| | | | TGCCAAAGTTACACATAGTACAGG | | |
| | | | TGGTGTGTAAGAAGTACCGGGG | | |
| | | | ATTCACCATCCCCGAGGCCTTCCG | | |
| | | | GGGAGTGCATCGGTACTTGAGCAA | | |
| | | | TGCCTACGCCCGGGAAGAATTC | | |
| | | | GCTTCCACCTGTCCAGATGATGAG | | |
| | | | GAGATCGAGCTCGCCTATGAGCAA | | |
| | | | GTGGCAAAGGCCCTCAAATAAG | | |
| | | | CCCCTCCTGGGACTCCCTCAACCC | | |
| | | | CCTCCATTTTCTCCACAAAGGCCCT | | |
| | | | GGTGGTTTCCACATTGCTACC | | |
| | | | CAATGGACACACTCCAAAATGGCC | | |
| | | | AGTGGGCAGGGAATCCTGGAGCAC | | |
| | | | TTGTTCCGGGATGGTGTGGTGG | | |
| | | | AAGAGGGGATGAGGGAAAGAAAT | | |
| | | | GGGGGGCCTGGGTCAGATTTTTAT | | |
| | | | TGTGGGGTGGGATGAGTAGGACA | | |
| | | | ACATATTTCAGTAATAAAATACAG | | |
| | | | AATAAAAATCAAGTGTTTTTACGC | | |
| | | | AAAAAAAAAAAAA (SEQ ID NO: 55) | | |
| ATP6V0E1 | ATPase H+ | NM_003945.4 | GCACACGCTGGTCACGCGGTCAGC | NP_003936.1 | MAYHGLTVPLIVMSVF |
| | Trans- | | TATTGACACTTCCTGGTGGGATCC | | WGFVGFLVPWFIPKGPN |
| | porting | | GAGTGAGGCGACGGGGTAGGGG | | RGVIITMLVTC |
| | V0 | | TTGGCGCTCAGGCGGCGACCATGG | | SVCCYLFWLIAILAQLN |
| | Subunit | | CGTATCACGGCCTCACTGTGCCTCT | | PLFGPQLKNETIWYLKY |
| | E1 | | CATTGTGATGAGCGTGTTCTG | | HWP (SEQ ID NO: 58) |
| | | | GGGCTTCGTCGGCTTCTTGGTGCCT | | |
| | | | TGGTTCATCCCTAAGGGTCCTAAC | | |
| | | | CGGGGAGTTATCATTACCATG | | |
| | | | TTGGTGACCTGTTCAGTTTGCTGCT | | |
| | | | ATCTCTTTTGGCTGATTGCAATTCT | | |
| | | | GGCCCAACTCAACCCTCTCT | | |
| | | | TTGGACCGCAATTGAAAAATGAAA | | |
| | | | CCATCTGGTATCTGAAGTATCATTG | | |
| | | | GCCTTGAGGAAGAAGACATGC | | |
| | | | TCTACAGTGCTCAGTCTTTGAGGTC | | |
| | | | ACGAGAAGAGAATGCCTTCTAGAT | | |
| | | | CCAAAATCACCTCCAAACCAG | | |
| | | | ACCACTTTTCTTGACTTGCCTGTTT | | |
| | | | TGGCCATTAGCTGCCTTAAACGTT | | |
| | | | AACAGCACATTTGAATGCCTT | | |
| | | | ATTCTACAATGCAGCGTGTTTTCCT | | |
| | | | TTGCCTTTTTTGCACTTTGGTGAAT | | |
| | | | TACGTGCCTCCATAACCTGA | | |
| | | | ACTGTGCCGACTCCACAAAACGAT | | |
| | | | TATGTACTCTTCTGAGATAGAAGA | | |
| | | | TGCTGTTCTTCTGAGAGATACG | | |
| | | | TTACTCTCTCCTTGGAATCTGTGGA | | |
| | | | TTTGAAGATGGCTCCTGCCTTCTCA | | |
| | | | CGTGGGAATCAGTGAAGTGT | | |
| | | | TTAGAAACTGCTGCAAGACAAACA | | |
| | | | AGACTCCAGTGGGGTGGTCAGTAG | | |
| | | | GAGAGCACGTTCAGAGGGAAGA | | |
| | | | GCCATCTCAACAGAATCGCACCAA | | |
| | | | ACTATACTTTCAGGATGAATTTCTT | | |
| | | | CTTTCTGCCATCTTTTGGAAT | | |
| | | | AAATATTTTCCTCCTTTCTATGGAA | | |
| | | | ATCTGGGCTCGGTGTTTGTAAAGTT | | |
| | | | CATTTTTATAAGCTTTTCTA | | |
| | | | TCGCTACATAATGCCTTTTTAAAAA | | |
| | | | ATGATTTTGTAGTCTAAACTTAGGT | | |
| | | | TGAGTATATAAACCCTGCCA | | |
| | | | TGTAGCTTGAGATGCCTGAAAAGA | | |
| | | | CTGGTAAGTGCGTTTCTTAATCGTT | | |
| | | | CAGTAACTATTTGAGTGCCTA | | |
| | | | CTGCAGCCAAGGCACTGGAGGGAT | | |
| | | | CAAAGATGTGTAAATTTGGAGTCC | | |
| | | | CTGCAAGTTCACAAGCTATTTG | | |
| | | | GAGAGATAAGGTTAGTATACATAG | | |
| | | | AACTGTAATATAAGGTTGTGTTGG | | |
| | | | AGCATTGTCCTTAAAGATGGTA | | |
| | | | CCATGGTGAGCAGTTCAAGGTTAC | | |
| | | | CTGCCAGCTGCAGAACAAGGCAGC | | |
| | | | AAATGCTCCTGAGATGGAACCA | | |
| | | | TCACAGCCTCAGACATAGGACTAA | | |
| | | | AGAAGTCAAGAGTGATTAAAAAGC | | |
| | | | CACGGGCACGAGACAGTAATTT | | |
| | | | TGTATTTCAGTAGCAGGCATCTCG | | |
| | | | ATACACTAATTTGAGAGCTTTATTA | | |
| | | | CTTTTAAGAAATTAAAAATTA | | |
| | | | AAATGAACCTAAATTTTCA (SEQ ID | | |
| | | | NO: 57) | | |
| NCL | Nucleolin | NM_005381.3 | AGTCTCGAGCTCTCGCTGGCCTTC | NP_005372.2 | MVKLAKAGKNQGDPK |
| | | | GGGTGTACGTGCTCCGGGATCTTC | | KMAPPPKEVEEDSEDEE |
| | | | AGCACCCGCGGCCGCCATCGCC | | MSEDEEDDSSCE |
| | | | GTCGCTTGGCTTCTTCTGGACTCAT | | EVVIPQKKGKKAAATS |
| | | | CTGCGCCACTTGTCCGCTTCACACT | | AKKVVVSPTKKVAVAT |
| | | | CCGCCGCCATCATGGTGAAG | | PAKKAAVTPGKKAAAT |
| | | | CTCGCGAAGGCAGGTAAAAATCAA | | PAKKTVTPAK |
| | | | GGTGACCCCAAGAAAATGGCTCCT | | AVTTPGKKGATPGKAL |
| | | | CCTCCAAAGGAGGTAGAAGAAG | | VATPGKKGAAIPAKGA |
| | | | ATAGTGAAGATGAGGAAATGTCAG | | KNGKNAKKEDSDEEED |
| | | | AAGATGAAGAAGATGATAGCAGT | | DDSEEDEEDD |
| | | | GGAGAAGAGCTCGTCATACCTCA | | EDEDEDEDEIEPAAMKA |
| | | | GAAGAAAGGCAAGAAGGCTGCTG | | AAAAPASEDEDDEDDE |
| | | | CAACCTCAGCAAAGAAGGTGGTCG | | DDEDDDDDEEDDSEEE |
| | | | TTTCCCCAACAAAAAAGGTTGCA | | AMETTPAKG |
| | | | GTTGCCACACCAGCCAAGAAAGCA | | KKAAKVVPVKAKNVAE |
| | | | GCTGTCACTCCAGGCAAAAAGGCA | | DEDEEEDDEDEDDDDD |
| | | | GCAGCAACACCTGCCAAGAAGA | | EDDEDDDDEDDEEEEEE |
| | | | CAGTTACACCAGCCAAAGCAGTTA | | EEEEPVKEA |
| | | | CCACACCTGGCAAGAAGGGAGCC | | PGKRKKEMAKQKAAPE |
| | | | ACACCAGGCAAAGCATTGGTAGC | | AKKQKVEGTEPTTAFNL |
| | | | AACTCCTGGTAAGAAGGGTGCTGC | | FVGNLNENKSAPELKTG |
| | | | CATCCCAGCCAAGGGGGCAAAGA | | ISDVFAKN |
| | | | ATGGCAAGAATGCCAAGAAGGAA | | DLAVVDVRIGMTRKFG |
| | | | GACAGTCATGAAGAGGAGGATGA | | YVDFESAEDLEKALELT |
| | | | TGACAGTGAGGAGGATGAGGAGG | | GLKVFGNEIKLEKPKGK |
| | | | ATGACGAGGACGAGGATGAGGAT | | DSKKERDA |
| | | | G | | RTLLAKNLPYKVTQDEL |
| | | | AAGATGAAATTGAACCAGCAGCGA | | KEVFEDAAEIRLVSKDG |
| | | | TGAAAGCAGCAGCTGCTGCCCCTG | | KSKGIAYIEFKTEADAE |
| | | | CCTCAGAGGATGAGGACGATGA | | KTFEEKQ |
| | | | GGATGACGAAGATGATGAGGATG | | GTEIDGRSISLYYTGEKG |
| | | | ACGATGACGATGAGGAAGATGACT | | QNQDYRGGKNSTWSGE |
| | | | CTGAAGAAGAAGCTATGGAGACT | | SKTLVLSNLSYSATEET |
| | | | ACACCAGCCAAAGGAAAGAAAGC | | LQEVFEK |
| | | | TGCAAAAGTTGTTCCTGTGAAAGC | | ATFIKVPQNQNGKSKG |
| | | | CAAGAACGTGGCTGAGGATGAAG | | YAFIEFASFEDAKEALN |
| | | | ATGAAGAAGAGGATGATGAGGAC | | SCNKREIEGRAIRLELQG |
| | | | GAGGATGACGACGACGACGAAGA | | PRGSPNA |
| | | | TGATGAAGATGATGATCATGAAGA | | RSQPSKTLFVKGLSEDT |
| | | | TGATGAGGAGGAGGAAGAAGAGG | | TEETLKESFDGSVRARI |
| | | | AGGAGGAAGAGCCTGTCAAAGAA | | VTDRETGSSKGFGFVDF |
| | | | GCACCTGGAAAACGAAAGAAGGA | | NSEEDAK |
| | | | A | | AAKEAMEDGEIDGNKV |
| | | | ATGGCCAAACAGAAAGCAGCTCCT | | TLDWAKPKGEGGFGGR |
| | | | GAAGCCAAGAAACAGAAAGTGGA | | GGGRGGFGGRGGGRGG |
| | | | AGGCACAGAACCGACTACGGCTT | | RGGFGGRGRG |
| | | | TCAATCTCTTTGTTGGAAACCTAAA | | GFGGRGGFRGGRGGGG |
| | | | CTTTAACAAATCTGCTCCTGAATTA | | DHKPQGKKTKFE (SEQ |
| | | | AAAACTGGTATCAGCGATGT | | ID NO: 60) |
| | | | TTTTGCTAAAAATGATCTTGCTGTT | | |
| | | | GTGGATGTCAGAATTGGTATGACT | | |
| | | | AGGAAATTTGGTTATGTGGAT | | |
| | | | TTTGAATCTGCTCAAGACCTGGAG | | |
| | | | AAAGCGTTGGAACTCACTGGTTTG | | |
| | | | AAAGTCTTTGGCAATGAAATTA | | |
| | | | AACTAGAGAAACCAAAAGGAAAA | | |
| | | | GACAGTAAGAAAGACCGAGATGC | | |
| | | | GAGAACACTTTTGGCTAAAAATCT | | |
| | | | CCCTTACAAAGTCACTCAGGATGA | | |
| | | | ATTGAAAGAAGTGTTTCAAGATGC | | |
| | | | TGCGGAGATCAGATTAGTCAGC | | |
| | | | AAGGATGGGAAAAGTAAAGGGAT | | |
| | | | TGCTTATATTGAATTTAAGACAGA | | |
| | | | AGCTGATGCAGAGAAAACCTTTG | | |
| | | | AAGAAAAGCAGGGAACAGAGATC | | |
| | | | GATGGGCGATCTATTTCGCTGTACT | | |
| | | | ATACTGGAGAGAAAGGTCAAAA | | |
| | | | TCAAGACTATAGAGGTGGAAAGAA | | |
| | | | TAGCACTTGGAGTGGTGAATCAAA | | |
| | | | AACTCTGGTTTTAAGCAACCTC | | |
| | | | TCCTACAGTGCAACAGAAGAAACT | | |
| | | | CTTCAGGAAGTATTTGAGAAAGCA | | |
| | | | ACTTTTATCAAAGTACCCCAGA | | |
| | | | ACCAAAATGGCAAATCTAAAGGGT | | |
| | | | ATGCATTTATAGAGTTTGCTTCATT | | |
| | | | CGAAGACGCTAAAGAAGCTTT | | |
| | | | AAATTCCTGTAATAAAAGGGAAAT | | |
| | | | TGAGGGCAGACCAATCAGGCTGGA | | |
| | | | GTTGCAAGGACCCAGGGGATCA | | |
| | | | CCTAATGCCAGAAGCCAGCCATCC | | |
| | | | AAAACTCTGTTTGTCAAACGCCTG | | |
| | | | TCTGAGGATACCACTGAAGAGA | | |
| | | | CATTAAAGGAGTCATTTGACGGCT | | |
| | | | CCGTTCGGGCAAGGATAGTTACTG | | |
| | | | ACCGGGAAACTGGGTCCTCCAA | | |
| | | | AGGGTTTGGTTTTGTAGACTTCAAC | | |
| | | | AGTGAGGAGGATGCCAAAGCTGCC | | |
| | | | AAGGAGGCCATGGAAGACGGT | | |
| | | | GAAATTGATGGAAATAAAGTTACC | | |
| | | | TTGGACTGGGCCAAACCTAAGGGT | | |
| | | | CAAGGTGGCTTCGGGGGTCGTG | | |
| | | | GTGGAGGCAGAGGCGGCTTTGGAG | | |
| | | | GACGAGGTGGTGGTAGAGGAGGC | | |
| | | | CGAGGAGGATTTGGTGGCAGAGG | | |
| | | | CCGGGGAGGCTTTGGAGGGCGAGG | | |
| | | | AGGCTTCCGAGGAGGCAGAGGAG | | |
| | | | GAGGAGGTGACCACAAGCCACAA | | |
| | | | GGAAAGAACACCAAGTTTGAATA | | |
| | | | GCTTCTGTCCCTCTGCTTTCCCTTTT | | |
| | | | CCATTTGAAAGAAAGGACTCT | | |
| | | | GGGGTTTTTACTGTTACCTGATCAA | | |
| | | | TGACAGAGCCTTCTGAGGACATTC | | |
| | | | CAAGACAGTATACAGTCCTGT | | |
| | | | GGTCTCCTTGGAAATCCGTCTAGTT | | |
| | | | AACATTTCAAGGCCAATACCGTCT | | |
| | | | TGGTTTTGACTGGATATTCAT | | |
| | | | ATAAACTTTTTAAAGAGTTGAGTG | | |
| | | | ATAGAGCTAACCCTTATCTGTAAG | | |
| | | | TTTTGAATTTATATTGTTTCAT | | |
| | | | CCCATGTACAAAACCATTTTTTCCT | | |
| | | | ACAAATAGTTTGGGTTTTGTTGTTG | | |
| | | | TTTCTTTTTTTTGTTTTGTT | | |
| | | | TTTGTTTTTTTTTTTTTTGCGTTCGT | | |
| | | | GGGGTTGTAAAACAAAAGAAAGC | | |
| | | | AGAATGTTTTATCATGGTTTT | | |
| | | | TGCTTCAGCGGCTTTAGGACAAAT | | |
| | | | TAAAAGTCAACTCTGGTGCCAGAC | | |
| | | | GTGTTACTTCCTAAAGAGTGTT | | |
| | | | TCCCCTGGAATGTCACTGGAGAGC | | |
| | | | ATGGCAAAGCCAGCTCTGCCACTT | | |
| | | | GCTTCACCCATCCCAATGGAAA | | |
| | | | TGGCTTAGTGCGTGTTTCCAGTATC | | |
| | | | CCAGCCCTAACTAACTTGGTTGAA | | |
| | | | ATGCTGGTGAGGGGACCTCCT | | |
| | | | CCTGCAGCCCTGGTGCTGACTTGA | | |
| | | | AGGCTGCTGCAGCTTCTCCTACTTT | | |
| | | | TAGCAGGTCTGAGGATTATGT | | |
| | | | CCTGAAGACCACTCTGGAAAGAGG | | |
| | | | TGCAGGAACAGATTAGTCAGGTTT | | |
| | | | CCTAGGACAAGGAAGAGCTTCA | | |
| | | | GGGAAGAGCAGTGGCTAACTCCTG | | |
| | | | TAATCCCAACACTTGGGGAGGCCG | | |
| | | | AGGCAGGCAGATCAACTGAGGT | | |
| | | | CAGGAGTTGAAGACCAGCCTGGCC | | |
| | | | AACATGGTGAAAGCCCATCTCTAC | | |
| | | | TAAAAATACAAAAATTAGCTGG | | |
| | | | GCATGGTGGTGTACTCCTGTAGTC | | |
| | | | CCAGCTACTCAGGAGGCTGAAGCG | | |
| | | | GGAGAGTCACGTCAACCCGGGA | | |
| | | | AGCAGAGTGAGCTGAGCACACACT | | |
| | | | ACTATACTCCAGGCTGGGTAACAA | | |
| | | | AGCGAGACTCCCATCTCCCAAA | | |
| | | | AAGCAGTTCTGGAATAGAACTCAC | | |
| | | | GCTAGATGGATAGACCAGTGGACA | | |
| | | | CTTTGGAACCTTGGGGCTGGGG | | |
| | | | AGGAAACTGCCCATCCAGTAAACC | | |
| | | | CCCAAAAAGCCATTTGTTCTGCAC | | |
| | | | TACGTATATTGCTTATTCTTTC | | |
| | | | TGGTCTTAAGTACTTGCCTCTCAAC | | |
| | | | CTCCCTTTTTACTAAAAGACAAGG | | |
| | | | CCACGTGAGAGGCGGGACTAT | | |
| | | | CAACATTGTGATCAATTTACTTCA | | |
| | | | AACCCAGTGCCCAAAATCAATGTA | | |
| | | | GGTAGCCAAGTCCAAAAACCTG | | |
| | | | TTCTAGTCCAACTAGTGAAATCAA | | |
| | | | ACTGTGATACTTGGATAAGCTTAG | | |
| | | | AAGGAAACGTGAAGAATACGTA | | |
| | | | CCTGCTTTGGGTTTACTCTGGTTCA | | |
| | | | GTTGGGCTGTTGAAATCTTAACAT | | |
| | | | CCTTGGGCTTATCACCTACTG | | |
| | | | CTTGTCAGCCCTGTTCCATGTCCAG | | |
| | | | GGGATGGGGGTGGTGACAATCCAG | | |
| | | | TTCCAAGACCCTCATGCTCTA | | |
| | | | GAGAGGAAGGTGGCCAGCCAGGG | | |
| | | | TTGTAACTACGATGAAAAAGCAGT | | |
| | | | GGGAGGGTCTCCTATGAGGCAAG | | |
| | | | CCTAAGGACAAAAAGGAAGGCCTT | | |
| | | | GCAGCCTGTATTCTGGATAAGGAA | | |
| | | | TTAAAAGCTCAGTTAATTGAAG | | |
| | | | CCCA (SEQ ID NO: 59) | | |
| CIRBP | Cold | NM_001280.2 | AGGATGTGTAGGGGGCGGGGCCCG | NP_001271.1 | MASDEGKLFVGGLSFD |
| | Inducible | | GCGGAAGCGTATATAAGGCCGGGC | | TNEQSLEQVFSKYGQIS |
| | RNA | | TCGGGGACCCCCCCCCCTCACT | | EVVVVKDRETQ |
| | Binding | | CGCGCGTTAGGAGGCTCGGGTCGT | | RSRGFGFVTFENIDDAK |
| | Protein | | TGTGGTGCGCTGTCTTCCCGCTTGC | | DAMMAMNGKSVDGRQ |
| | | | GTCAGGGACCTGCCCGACTCA | | IRVDQAGKSSDNRSRGY |
| | | | GTGGCCGCCATGCCATCAGATGAA | | RGGSAGGRG |
| | | | GGCAAACTTTTTGTTGGAGGGCTG | | FFRGGRGRGRGFSRGG |
| | | | AGTTTTGACACCAATGAGCAGT | | GDRGYGGNRFESRSGG |
| | | | CGCTGGAGCAGGTCTTCTCAAAGT | | YGGSRDYYSSRSQSGG |
| | | | ACGGACAGATCTCTGAAGTGGTGG | | YSDRSSGGSY |
| | | | TTGTGAAAGACAGGGAGACCCA | | RDSYDSYATHNE (SEQ |
| | | | GAGATCTCGGGGATTTGGGTTTGT | | [D NO: 62) |
| | | | CACCTTTGAGAACATTGACGACGC | | |
| | | | TAAGGATGCCATGATGGCCATG | | |
| | | | AATGGGAAGTCTGTAGATGGACGG | | |
| | | | CAGATCCGAGTAGACCAGGCAGGC | | |
| | | | AAGTCGTCAGACAACCGATCCC | | |
| | | | GTGGGTACCGTGGTGGCTCTGCCG | | |
| | | | GGGGCCGGGGCTTCTTCCGTGGGG | | |
| | | | GCCGAGGACGGGGCCGTGGGTT | | |
| | | | CTCTAGAGGAGGAGGGGACCGAG | | |
| | | | GCTATGGGGGGAACCGGTTCGAGT | | |
| | | | CCAGGAGTGGGGGCTACGGAGGC | | |
| | | | TCCAGAGACTACTATAGCAGCCGG | | |
| | | | AGTCAGAGTGGTGGCTACAGTGAC | | |
| | | | CGGAGCTCGGGCGGGTCCTACA | | |
| | | | GAGACAGTTATGACAGTTACGCTA | | |
| | | | CACACAACGAGTAAAAACCCTTCC | | |
| | | | TGCTCAAGATCGTCCTTCCAAT | | |
| | | | GGCTGTGTGTTTAAAGATTGTGGG | | |
| | | | AGCTTCGCTGAACGTTAATGTGTA | | |
| | | | GTAAATGCACCTCCTTGTATTC | | |
| | | | CCACTTTCGTAGTCATTTCGGTTCT | | |
| | | | GATCTTGTCAAACCCAGCCTGACC | | |
| | | | GCTTCTGACGCCGGGATGGCC | | |
| | | | TCGTTACTAGACTTTTCTTTTTAAG | | |
| | | | GAAGTGCTGTTTTTTTTTGAGGGTT | | |
| | | | TTCAAAACATTTTGAAAAGC | | |
| | | | ATTTACTTTTTTGACCACGAGCCAT | | |
| | | | CAGTTTTCAAAAAAATCGGGGGTT | | |
| | | | GTGTGGGTTTTTGGTTTTTGT | | |
| | | | TTTAGTTTTTGGTTGCGTTGCCTTT | | |
| | | | TTTTTTTTAGTGGGGTTGGCCCCAT | | |
| | | | GAAGTGGGTGCCCCACTCAC | | |
| | | | TTCTCTGAGATCGAACGGACTGTG | | |
| | | | AATCCGCTCTTTGTCGGAAGCTGA | | |
| | | | GCAAGCTGTGGCTTTTTTCCAA | | |
| | | | CTCCGTGTGACGTTTCTGAGTGTAG | | |
| | | | TGTGGTAGGACCCCGGCGGGTGTG | | |
| | | | GCAGCAACTGCCCTGGAGCCC | | |
| | | | CAGCCCCTGCGTCCATCTGTGCTGT | | |
| | | | GCGCCCCACAGTAGACGTGCAGAC | | |
| | | | GTCCCTGAGAGGTTCTTGAAG | | |
| | | | ATGTTTATTTATATTGTCCTTTTTTA | | |
| | | | CTGGAAGACGTACGCATACTCCAT | | |
| | | | CGATGTTGTATTTGCAGTGG | | |
| | | | CTGAGGAATTCTTGTACGCAGTTTT | | |
| | | | CTTTGGCTTTACGAAGCCGATTAA | | |
| | | | AAGACCGTGTGAAATGAA (SEQ ID | | |
| | | | NO: 61) | | |
| | | NM_001300815.2 | CTCACTCGCGCGTTAGGAGGCTCG | NP_001287744.1 | MASDEGKLFVGGLSFD |
| | | | GGTCGTTGTGGTGCGCTGTCTTCCC | | TNEQSLEQVFSKYGQIS |
| | | | GCTTGCGTCAGGGACCTGCCC | | EVVVVKDRETQ |
| | | | GACTCAGTGGCCCCCATGGCATCA | | RSRGFGFVTFENIDDAK |
| | | | GATGAAGGCAAACTTTTTGTTGGA | | DAMMAMNGKSVDGRQ |
| | | | GGGCTGAGTTTTGACACCAATG | | IRVDQAGKSSDNRSRGY |
| | | | AGCAGTCGCTGGAGCAGGTCTTCT | | RGGSAGORG |
| | | | CAAAGTACGGACAGATCTCTGAAG | | FFRGGRGRGRGFSRGG |
| | | | TGGTGGTTGTGAAAGACAGGGA | | GDRGYGGNRFESRSGG |
| | | | GACCCAGAGATCTCGGGGATTTGG | | YGGSRDYYSSRSQSGG |
| | | | GTTTGTCACCTTTGAGAACATTGAC | | YSDRSSGGSY |
| | | | GACGCTAAGGATGCCATGATG | | RDSYDSYG (SEQ ID NO: |
| | | | GCCATGAATGGGAAGTCTGTAGAT | | 64) |
| | | | GGACGGCAGATCCGAGTAGACCA | | |
| | | | GGCAGGCAAGTCGTCAGACAACC | | |
| | | | GATCCCGTGGGTACCGTGGTGGCT | | |
| | | | CTGCCGGGGGCCGGGGCTTCTTCC | | |
| | | | GTGGGGGCCGAGGACGGGGCCG | | |
| | | | TGGGTTCTCTAGAGGAGGAGGGGA | | |
| | | | CCGAGGCTATGGGGGGAACCGGTT | | |
| | | | CGAGTCCAGGAGTGGGGGCTAC | | |
| | | | GGAGGCTCCAGAGACTACTATAGC | | |
| | | | AGCCGGAGTCAGAGTGGTGGCTAC | | |
| | | | AGTGACCGGAGCTCGGGCGGGT | | |
| | | | CCTACAGAGACAGTTATGACAGTT | | |
| | | | ACGGTTGAAGGGGCCCGGCCAGG | | |
| | | | ACTCGGGGAAGGGTGGCCTGAGA | | |
| | | | CCAGCGATGACCTCTGGGGTCACT | | |
| | | | GTCCCAGGAGGGACTTCACCTGGA | | |
| | | | ACAAGAGCTGGAGGCAGCCCCT | | |
| | | | TGGCCACGAGGCTTGTCCCCTGTA | | |
| | | | AGTGCTTTCGGGAAGAGTGGCATG | | |
| | | | TGGCGCTGAGCCCTGTCCCGGG | | |
| | | | CGGCACCTGGGCGTTTCAGTGAGT | | |
| | | | CCTGCTCTCCCGCACCTATGGCCCC | | |
| | | | ATGGCGGGCGCCTTTCGGTGT | | |
| | | | GTGTTGGGTGCAGGGCAGCGCCTC | | |
| | | | CCGGGAGCGCCGGGTCCCCCGCCT | | |
| | | | GGAGCCCGCGCCTGTTCTCCCT | | |
| | | | CCCTTCCTCCTCCTTCCAGGAGGCG | | |
| | | | CTTCGCCAGTGAGGTGCGGGCTCA | | |
| | | | GGGCCTCGAGTCTCTCCTGGA | | |
| | | | GCACGGGCTGCGGTGCGCCGGCAG | | |
| | | | CTTACGGGGCGGCCAGTCCTTGCC | | |
| | | | CACAACGATGTOGAGCCCTGTG | | |
| | | | AAAGTCGGATTCGAATAAAGGGCC | | |
| | | | ACGTGTGCACCCAGAAA (SEQ ID | | |
| | | | NO: 63) | | |
| | | NM_001300829.2 | CTCACTCGCGCGTTAGGAGGCTCG | NP_001287758.1 | MASDEGKLFVGGLSFD |
| | | | GGTCGTTGTGGTGCGCTGTCTTCCC | | TNEQSLEQVESKYGQIS |
| | | | CCTTGCGTCAGGGACCTGCCC | | EVVVVKDRETQ |
| | | | GACTCAGTGGCCCCCATGGCATCA | | RSRGFGFVTFENIDDAK |
| | | | GATGAAGGCAAACTTTTTGTTGGA | | DAMMAMNGKSVDGRQ |
| | | | GGGCTGAGTTTTGACACCAATG | | IRVDQAGKSSDNRSRGY |
| | | | AGCAGTCGCTGGAGCAGGTCTTCT | | RGGSAGGRG |
| | | | CAAAGTACGGACAGATCTCTGAAG | | FFRGGRGRGRGFSRGG |
| | | | TGGTGGTTGTGAAAGACAGGGA | | GDRGYGGNRFESRSGG |
| | | | GACCCAGAGATCTCGGGGATTTGG | | YGGSRDYYSSRSQSGG |
| | | | GTTTGTCACCTTTGAGAACATTGAC | | YSDRSSGGSY |
| | | | GACGCTAAGGATGCCATGATG | | RDSYDSYGKSHSEGATL |
| | | | GCCATGAATGGGAAGTCTGTAGAT | | LWPAVGARFILVPSPST |
| | | | GGACGGCACATCCGAGTAGACCA | | LGWTLRPCHCACPEEA |
| | | | GGCAGGCAAGTCGTCAGACAACC | | HLSSQSHF |
| | | | GATCCCGTGGGTACCGTGGTGGCT | | YRRTQKPNETDQKGKG |
| | | | CTGCCGGGGGCCGGGGCTTCTTCC | | ERGPAGQSARCMCGRR |
| | | | GTGGGGGCCGAGGACGGGGCCG | | PASLGCGGWLLPGRRP |
| | | | TGGGTTCTCTAGAGGAGGAGGGGA | | RPGLASGVKL |
| | | | CCGAGGCTATGGGGGGAACCGGTT | | PLVASVPLHCACFLSSA |
| | | | CGAGTCCAGGAGTGGGGGCTAC | | THNE (SEQ ID NO: 66) |
| | | | GGAGGCTCCAGAGACTACTATAGC | | |
| | | | AGCCGGAGTCAGAGTGGTGCCTAC | | |
| | | | AGTGACCGGAGCTCGGGCGGGT | | |
| | | | CCTACAGAGACAGTTATGACAGTT | | |
| | | | ACGGTAAGTCACACTCCGAGGGCG | | |
| | | | CCACGCTGCTGTGGCCTGCGGT | | |
| | | | GGGAGCTCGGTTCACCTTGGTGCC | | |
| | | | CTCTCCAAGCACTTTAGGCTGGAC | | |
| | | | ACTCAGACCTTGTCACTGTGCT | | |
| | | | TGCCCAGAAGAGGCGCATCTGTCC | | |
| | | | TCTCAGAGCCATTTCTATCGCAGG | | |
| | | | ACGCAAAAGCCAAATGAGACTG | | |
| | | | ACCAAAAAGGCAAGGGAGAGCGA | | |
| | | | GGGCCCGCTGGGCAGTTCAGCTAGG | | |
| | | | TGCATGTGTGGCCGCAGGCCAGC | | |
| | | | CTCCCTCGGCTGTGGGGGGTGGTT | | |
| | | | GCTCCCCGGCCGCAGGCCGCGCCC | | |
| | | | TGGTCTGGCCTCTGGGGTGAAG | | |
| | | | CTGCCTCTTGTTGCTTCGGTGCCTT | | |
| | | | TACACTGTGCCTGCTTCTTGTCCTC | | |
| | | | AGCTACACACAACGAGTAAA | | |
| | | | AACCCTTCCTGCTCAAGATCGTCCT | | |
| | | | TCCAATGGCTGTGTCTTTAAAGATT | | |
| | | | GTGGGAGCTTCGCTGAACGT | | |
| | | | TAATGTGTAGTAAATGCACCTCCTT | | |
| | | | GTATTCCCACTTTCGTAGTCATTTC | | |
| | | | GGTTCTCATCTTGTCAAACC | | |
| | | | CAGCCTGACCGCTTCTGACGCCGG | | |
| | | | GATGGCCTCGTTACTAGACTTTTCT | | |
| | | | TTTTAAGGAAGTGCTGTTTTT | | |
| | | | TTTTGAGGGTTTTCAAAACATTTTG | | |
| | | | AAAAGCATTTACTTTTTTGACCACG | | |
| | | | AGCCATGACTTTTCAAAAAA | | |
| | | | ATCGGGGGTTGTGTGGGTTTTTGGT | | |
| | | | TTTTGTTTTAGTTTTTGGTTGCGTT | | |
| | | | GCCTTTTTTTTTTTAGTGGG | | |
| | | | GTTGGCCCCATGAAGTGGGTGCCC | | |
| | | | CACTCACTTCTCTGAGATCGAACG | | |
| | | | GACTGTGAATCCGCTCTTTGTC | | |
| | | | CGAAGCTGAGCAAGCTGTGGCTTT | | |
| | | | TTTCCAACTCCGTGTGACGTTTCTG | | |
| | | | AGTGTAGTGTGGTAGGACCCC | | |
| | | | GGCGGGTGTGGCAGCAACTGCCCT | | |
| | | | GGAGCCCCAGCCCCTGCGTCCATC | | |
| | | | TGTGCTGTGCGCCCCACAGTAG | | |
| | | | ACGTGCAGACGTCCCTGAGAGGTT | | |
| | | | CTTGAAGATGTTTATTTATATTGTC | | |
| | | | CTTTTTTACTGGAAGACGTAC | | |
| | | | GCATACTCCATCGATGTTGTATTTG | | |
| | | | CAGTGGCTGAGGAATTCTTGTACG | | |
| | | | CAGTTTTCTTTGGCTTTACGA | | |
| | | | AGCCGATTAAAAGACCGTGTGAAA | | |
| | | | TGAACCTTGCTCTGACAATTCCCTT | | |
| | | | GCATTGCACCACACACTCCTT | | |
| | | | GCTGCGGGCTCCTGCAGCCAGACC | | |
| | | | TGAGCAGAGAGAGAAGGTGGAGA | | |
| | | | AGCAGCGGGTCTGCAAGCCTTCC | | |
| | | | CTGGGGCCTGCAGAGCTAGAAAGG | | |
| | | | GAGGCCCAGCAGACTGGCGCTGGT | | |
| | | | CAGGGTAGGGGAGCCAGGCGGC | | |
| | | | GGACGGGAGCGGGCAGCTCAGGC | | |
| | | | CTCAGGGCAGCCCTGGGAGGCTTC | | |
| | | | TGGCAGTGGTGGCCAGAGGGCTG | | |
| | | | GACTGTGCGGGCAGCTTAGCAGGG | | |
| | | | ACAGTGGACGTGCACCTGACGCTG | | |
| | | | ACCTGGACTGCCTCAGTCTAGA | | |
| | | | AGCAGGCCAGAGAGCAGAGGCAC | | |
| | | | GTGGCATCCCAGGGCGACCTCAGA | | |
| | | | CGGCCAGCCGGTTAGCTAGTTCT | | |
| | | | GCTGTTCCTTCACGAGTTCTGAGC | | |
| | | | ATTCTCTGCTAGCCTATGGAAGCT | | |
| | | | GCAGCCCTCGGAGGACAGAAGT | | |
| | | | GTTGTGCGCCCAACAGAACCCTCT | | |
| | | | GAGACGCAAGCTGCTCCCTTGGCT | | |
| | | | AGCTCATATGTGGAAATAGCCC | | |
| | | | TGTAATTCGAGGTAACTCCTTCCGC | | |
| | | | TCGTGTCCACATCCCTCTTGTTGAG | | |
| | | | AGCTCACTGAAAGTCATGTG | | |
| | | | CCCGGGGAATGTTCCTGTGACTGT | | |
| | | | TTTTTGTTTTTCCTTTTTTTTTTAAC | | |
| | | | TTTGTTTTTGTTTTTTTCAA | | |
| | | | TTAAGCTGGAACTAAAGTCAGGCC | | |
| | | | CACCCATTACGCTCCCCACGTCCA | | |
| | | | CCCACGTGCAGCCTGGGCCCAG | | |
| | | | TCATGCCTGGCTCATAGATGAAAT | | |
| | | | CCCTTAAGCAGGATTGAAGACCAG | | |
| | | | TGAACGCCCCCGCCTTTTGGAT | | |
| | | | TTTTTGCTCAATTGACCGTCTTTTC | | |
| | | | CAGACCTCTTTAAGTCACACTCTTA | | |
| | | | ACTTAGCTTTCTCTGATGTC | | |
| | | | TGTTGCCGCCATTAGTTTTTTTCTA | | |
| | | | GAGCCCACACTGGCCCACATAGCT | | |
| | | | CCATCCCATACGGGTAGCTGG | | |
| | | | CTCCAGCTGCGCCAAGGTGCAGAC | | |
| | | | CCGCCCTGGGCATGCTGGCCTGTG | | |
| | | | ACGGAGCCTGAGTCACAGCCC | | |
| | | | CCTGACTAGCCTGAGACCTTCCTA | | |
| | | | GGGGCTGTGGCTGTTTCCGGGGAG | | |
| | | | GCCGGGAGGGGCAGCTGTGAGC | | |
| | | | CCTGTGGAGGACGTTGGGAGTAAC | | |
| | | | GCTGCTTTGCTTTGGCAGGTTGAA | | |
| | | | GGGGCCCGGCCAGGACTCGGGG | | |
| | | | AAGGGTGGCCTGAGAGCAGCGATG | | |
| | | | ACCTCTGGGGTCACTGTCCCAGGA | | |
| | | | GGGACTTCACCTGGAACAAGAG | | |
| | | | CTGGAGGCAGCCGCTTGCCCAGGA | | |
| | | | GGCTTGTCCCCTGTAAGTGCTTTCG | | |
| | | | GGAAGAGTGGCATGTGGCGCT | | |
| | | | GAGCCCTGTCCCGGGCGGCACCTG | | |
| | | | GGCGTTTCAGTGAGTCCTGCTCTCC | | |
| | | | CGCACCTATGGCCCCATGGCG | | |
| | | | GGCGCCTTTCGGTGTGTGTTGGGT | | |
| | | | GCAGGGCAGCGCCTCCCGGGAGCG | | |
| | | | CCGGGTCCCCCGCCTGGAGCCC | | |
| | | | GCGCCTGTTCTCCCTCCCTTCCTCC | | |
| | | | TCCTTCCAGGAGGCGCTTCGCCAG | | |
| | | | TGAGGTGCGGGCTCAGGGCCT | | |
| | | | CGAGTCTCTCCTGGAGCACGGGCT | | |
| | | | GCGGTGCGCCGGCAGCTTACGGGG | | |
| | | | CGCCCACTCCTTGCCCACAACG | | |
| | | | ATGTGGAGCCCTGTGAAAGTCGGA | | |
| | | | TTCGAATAAAGGGCCACGTGTGCA | | |
| | | | CCCAGAAA (SEQ ID NO: 65) | | |
| HSP90AB1 | Heat | NM_001271969.1 | TTTTTCGGACCATGACGTCAAGGT | NP_001258898.1 | MPEEVHHGEEEVETFAF |
| | Shock | | GGGCTGGTGGCGCCAGGTGCGGGG | | QAEIAQLMSLIINTFYSN |
| | Protein 90 | | TTGACAATCATACTCCTTTAAG | | KEIFLRELI |
| | Alpha | | GCGGAGGGATCTACAGGAGGGCG | | SNASDALDKIRYESLTD |
| | Family | | GCTGTACTGTGCTTCGCCTTATATA | | PSKLDSGKELKIDIIPNP |
| | Class B | | GGGCGACTTGGGGCACGCAGTA | | QERTLTLVDTGIGMIKA |
| | Member 1 | | GOTCTCTCGAGTCACTCCGGCGCA | | DLINNL |
| | | | GTGTTGGGACTGTCTGGGTATCGG | | GTIAKSGTKAFMEALQA |
| | | | AAAGCAAGCCTACGTTGCTCAC | | GADISMIGQFGVGFYSA |
| | | | TATTACGTATAATCCTTTTCTTTTC | | YLVAEKVVVITKHNDD |
| | | | AAGATTTTTATTTTAGATGCCTGAG | | EQYAWESS |
| | | | GAAGTGCACCATGGAGAGGA | | AGGSFTVRADHGEPIGR |
| | | | GGAGGTGGAGACTTTTGCCTTTCA | | GTKVILHLKEDQTEYLE |
| | | | GGCAGAAATTGCCCAACTCATGTC | | ERRVKEVVKKHSQFIGY |
| | | | CCTCATCATCAATACCTTCTAT | | PITLYLE |
| | | | TCCAACAAGGAGATTTTCCTTCGG | | KEREKEISDDEAEEEKG |
| | | | GAGTTGATCTCTAATGCTTCTGATG | | EKEEEDKDDEEKPKIED |
| | | | CCTTGGACAAGATTCGCTATG | | VGSDEEDDSGKDKKKK |
| | | | AGAGCCTGACAGACCCTTCGAAGF | | TKKIKEKY |
| | | | TGGACAGTGGTAAAGAGCTGAAAA | | IDQEELNKTKPIWTRNP |
| | | | TTGACATCATCCCCAACCCTCA | | DDITQEEYGEFYKSLTN |
| | | | GGAACGTACCCTGACTTTGGTAGA | | DWEDHLAVKHFSVEGQ |
| | | | CACAGGCATTGGCATGACCAAAGC | | LEFRALLF |
| | | | TGATCTCATAAATAATTTGGGA | | IPRRAPFDLFENKKKKN |
| | | | ACCATTGCCAAGTCTGGTACTAAA | | NIKLYVRRVFIMDSCDE |
| | | | GCATTCATGGAGGCTCTTCAGGCT | | LIPEYLNFIRGVVDSEDL |
| | | | GGTGCAGACATCTCCATGATTG | | PLNISR |
| | | | GGCAGTTTGGTGTTGGCTTTTATTC | | EMLQQSKILKVIRKNIV |
| | | | TGCCTACTTGGTGGCAGAGAAAGT | | KKCLELFSELAEDKENY |
| | | | GGTTGTGATCACAAAGCACAA | | KKFYEAFSKNLKLGTHE |
| | | | CGATGATGAACAGTATGCTTGGGA | | DSTNRRR |
| | | | GTCTTCTGCTGGAGGTTCCTTCACT | | LSELLRYHTSQSGDEMT |
| | | | CTGCGTCCTGACCATGGTGAG | | SLSEYVSRMKETQKSIY |
| | | | CCCATTGGCAGGGGTACCAAAGTG | | YITGESKEQVANSAFVE |
| | | | ATCCTCCATCTTAAAGAAGATCAG | | RVRKRGF |
| | | | ACAGAGTACCTAGAAGAGAGGC | | EVVYMTEPIDEYCVQQL |
| | | | GGGTCAAAGAAGTAGTGAAGAAG | | KEFDGKSLVSVTKEGLE |
| | | | CATTCTCAGTTCATAGGCTATCCCA | | LPEDEEEKKKMEESKA |
| | | | TCACCCTTTATTTGGAGAAGGA | | KFENLCKL |
| | | | ACGAGAGAAGGAAATTAGTGATG | | MKEILDKKVEKVTISNR |
| | | | ATGAGGCAGAGGAAGAGAAAGGT | | LVSSPCCIVTSTYGWTA |
| | | | GAGAAAGAAGAGGAAGATAAAGA | | NMERIMKAQALRDNST |
| | | | T | | MGYMMAKK |
| | | | GATGAAGAAAAACCCAAGATCGA | | HLEINPDHPIVETLRQKA |
| | | | AGATGTGGGTTCAGATGAGGAGGA | | EADKNDKAVKDLVVLL |
| | | | TGACAGCGGTAAGGATAAGAAGA | | FETALLSSGFSLEDPQTH |
| | | | AGAAAACTAAGAAGATCAAAGAG | | SNRIYR |
| | | | AAATACATTGATCAGGAAGAACTA | | MIKLGLGIDEDEVAAEE |
| | | | AACAAGACCAAGCCTATTTGGAC | | PNAAVPDEIPPLEGDED |
| | | | CAGAAACCCTGATGACATCACCCA | | ASRMEEVD (SEQ ID NO: |
| | | | AGAGGAGTATGGAGAATTCTACAA | | 67) |
| | | | GAGCCTCACTAATGACTGGGAA | | |
| | | | CACCACTTGGCAGTCAAGCACTTT | | |
| | | | TCTGTAGAAGGTCAGTTGGAATTC | | |
| | | | AGGGCATTGCTATTTATTCCTC | | |
| | | | GTCGGGCTCCCTTTGACCTTTTTGA | | |
| | | | GAACAAGAAGAAAAACAACAACA | | |
| | | | TCAAACTCTATGTCCGCCGTGT | | |
| | | | GTTCATCATGGACACCTGTGATGA | | |
| | | | GTTGATACCAGAGTATCTCAATTTT | | |
| | | | ATCCGTGGTGTGGTTGACTCT | | |
| | | | GAGGATCTGCCCCTGAACATCTCC | | |
| | | | CGAGAAATGCTCCAGCAGACCAA | | |
| | | | AATCTTGAAAGTCATTCGCAAAA | | |
| | | | ACATTGTTAAGAAGTGCCTTGAGC | | |
| | | | TCTTCTCTGAGCTGGCAGAAGACA | | |
| | | | AGGAGAATTACAAGAAATTCTA | | |
| | | | TGAGGCATTCTCTAAAAATCTCAA | | |
| | | | GCTTGGAATCCACGAAGACTCCAC | | |
| | | | TAACCGCCGCCGCCTGTCTGAG | | |
| | | | CTGCTGCGCTATCATACCTCCCAGT | | |
| | | | CTGGAGATGAGATGACATCTCTGT | | |
| | | | CAGAGTATGTTTCTCGCATGA | | |
| | | | AGGAGACACAGAAGTCCATCTATT | | |
| | | | ACATCACTGGTGAGAGCAAAGAGC | | |
| | | | AGGTGGCCAACTCAGCTTTTGT | | |
| | | | GGAGCGAGTGCGGAAACGGGGCTT | | |
| | | | CGAGGTGGTATATATGACCGAGCC | | |
| | | | CATTGACGAGTACTGTGTGCAG | | |
| | | | CACCTCAAGGAATTTGATGGGAAG | | |
| | | | AGCCTGGTCTCAGTTACCAAGGAG | | |
| | | | GGTCTGGAGCTGCCTGAGGATG | | |
| | | | AGGAGGAGAAGAAGAAGATGGAA | | |
| | | | GAGAGCAAGGCAAAGTTTGAGAA | | |
| | | | CCTCTGCAAGCTCATGAAAGAAAT | | |
| | | | CTTAGATAAGAAGGTTGAGAAGGT | | |
| | | | GACAATCTCCAATAGACTTGTGTC | | |
| | | | TTCACCTTGCTGCATTGTGACC | | |
| | | | AGCACCTACGGCTGCACAGCCAAT | | |
| | | | ATGGAGCGGATCATGAAAGCCCAG | | |
| | | | GCACTTCGGGACAACTCCACCA | | |
| | | | TGGGCTATATGATGGCCAAAAAGC | | |
| | | | ACCTGGAGATCAACCCTGACCACC | | |
| | | | CCATTGTGGAGACGCTGCGGCA | | |
| | | | GAAGGCTGAGGCCGACAAGAATG | | |
| | | | ATAAGGCAGTTAAGGACCTGGTGG | | |
| | | | TGCTGCTGTTTCAAACCGCCCTG | | |
| | | | CTATCTTCTGGCTTTTCCCTTGAGG | | |
| | | | ATCCCCAGACCCACTCCAACCGCA | | |
| | | | TCTATCGCATGATCAACCTAG | | |
| | | | CTCTAGGTATTGATGAAGATGAAG | | |
| | | | TGGCAGCAGAGGAACCCAATGCTG | | |
| | | | CAGTTCCTGATGAGATCCCCCC | | |
| | | | TCTCGAGGGCGATGAGGATGCGTC | | |
| | | | TCGCATGGAAGAAGTCGATTAGGT | | |
| | | | TAGGAGTTCATAGTTGGAAAAC | | |
| | | | TTGTGCCCTTGTATAGTGTCCCCAT | | |
| | | | GGGCTCCCACTGCAGCCTCGAGTG | | |
| | | | CCCCTGTCCCACCTGGCTCCC | | |
| | | | CCTGCTGGTGTCTAGTGTTTTTTTC | | |
| | | | CCTCTCCTGTCCTTGTGTTGAAGGC | | |
| | | | AGTAAACTAAGGGTGTCAAG | | |
| | | | CCCCATTCCCTCTCTACTCTTGACA | | |
| | | | CCAGGATTGGATGTTGTGTATTGT | | |
| | | | GGTTTATTTTATTTTCTTCAT | | |
| | | | TTTGTTCTGAAATTAAAGTATGCA | | |
| | | | AAATAAAGAATATGCCGTTTTTAT | | |
| | | | ACAGTTCT (SEQ ID NO: 67) | | |
| | | NM_007355.4 | CTCTCGAGTCACTCCGGCGCAGTG | NP_031381.2 | MPEEVHHGEEEVETFAF |
| | | | TTGGGACTGTCTGGGTATCGGAAA | | QAEIAQLMSLIINTFYSN |
| | | | GCAAGCCTACGTTGCTCACTAT | | KEIFLRELI |
| | | | TACGTATAATCCTTTTCTTTTCAAG | | SNASDALDKIRYESLTD |
| | | | ATGCCTGAGGAAGTGCACCATGGA | | PSKLDSGKELKIDIIPNP |
| | | | GAGGAGGAGGTGGAGACTTTT | | QERTLTLVDTGIGMTKA |
| | | | GCCTTTCAGGCAGAAATTGCCCAA | | DLINNL |
| | | | CTCATGTCCCTCATCATCAATACCT | | GTIAKSGTKAFMEALQA |
| | | | TCTATTCCAACAAGGAGATTT | | GADISMIGQFGVGFYSA |
| | | | TCCTTCGGGAGTTGATCTCTAATGC | | YLVAEKVVVITKHNDD |
| | | | TTCTGATGCCTTGGACAAGATTCG | | EQYAWESS |
| | | | CTATGAGAGCCTGACAGACCC | | AGGSFTVRADHGEPIGR |
| | | | TTCGAAGTTGGACAGTGGTAAAGA | | GTKVILHLKEDQTEYLE |
| | | | GCTGAAAATTGACATCATCCCCAA | | ERRVKEVVKKHSQFIGY |
| | | | CCCTCAGGAACGTACCCTGACT | | PITLYLE |
| | | | TTGGTAGACACAGGCATTGGCATG | | KEREKEISDDEAEEEKG |
| | | | ACCAAAGCTGATCTCATAAATAAT | | EKEEEDKDDEEKPKIED |
| | | | TTGGGAACCATTGCCAAGTCTG | | VGSDEEDDSCKDKKKK |
| | | | GTACTAAAGCATTCATGGAGGCTC | | TKKIKEKY |
| | | | TTCAGGCTGGTGCAGACATCTCCA | | IDQEELNKTKPIWTRNP |
| | | | TGATTGGGCAGTTTGGTGTTGG | | DDITQEEYGEFYKSLTN |
| | | | CTTTTATTCTGCCTACTTGGTGGCA | | DWEDHLAVKHFSVEGQ |
| | | | CAGAAAGTGGTTCTGATCACAAAG | | LEFRALLF |
| | | | CACAACGATGATGAACAGTAT | | IPRRAPFDLFENKKKKN |
| | | | GCTTGGGAGTCTTCTGCTGGAGGT | | NIKLYVRRVFIMDSCDE |
| | | | TCCTTCACTGTGCGTGCTGACCATG | | LIPEYLNFIRGVVDSEDL |
| | | | GTGAGCCCATTGGCAGGGGTA | | PLNISR |
| | | | CCAAAGTGATCCTCCATCTTAAAG | | EMLQQSKILKVIRKNIV |
| | | | AAGATCAGACAGAGTACCTAGAAG | | KKCLELFSELAEDKENY |
| | | | AGAGGCGGGTCAAAGAAGTAGT | | KKFYEAFSKNLKLGIHE |
| | | | GAAGAAGCATTCTCAGTTCATAGG | | DSTNRRR |
| | | | CTATCCCATCACCCTTTATTTGGAG | | LSELLRYHTSQSGDEMT |
| | | | AAGGAACGAGAGAAGGAAATT | | SLSEYVSRMKETQKSIY |
| | | | AGTGATGATGAGGCAGAGGAAGA | | YITGESKEQVANSAFVE |
| | | | GAAAGGTGAGAAAGAAGAGGAAG | | RVRKRGF |
| | | | ATAAAGATCATGAAGAAAAACCC | | EVVYMTEPIDEYCVQQL |
| | | | A | | KEFDGKSLVSVTKEGLE |
| | | | AGATCGAAGATGTGGGTTCAGATG | | LPEDEEEKKKMEESKA |
| | | | AGGAGGATGACAGCGGTAAGGAT | | KFENLCKL |
| | | | AAGAAGAAGAAAACTAAGAAGAT | | MKEILDKKVEKVTISNR |
| | | | CAAAGAGAAATACATTCATCAGGA | | LVSSPCCIVISTYGWTA |
| | | | AGAACTAAACAAGACCAAGCCTAT | | NMERIVKAQALRDNST |
| | | | TTGGACCACAAACCCTGATGAC | | MGYMMAKK |
| | | | ATCACCCAAGAGGAGTATGGAGAA | | HLEINPDHPIVETLRQKA |
| | | | TTCTACAAGAGCCTCACTAATGAC | | EADKNDKAVKDLVVLL |
| | | | TGGGAAGACCACTTGGCAGTCA | | FETALLSSGFSLEDPQTH |
| | | | AGCACTTTTCTGTAGAAGGTCAGT | | SNRIYR |
| | | | TGGAATTCAGGGCATTGCTATTTAT | | MIKLGLGIDEDEVAAEE |
| | | | TCCTCGTCGGGCTCCCTTTGA | | PNAAVPDEIPPLEGDED |
| | | | CCTTTTTGAGAACAAGAAGAAAAA | | ASRMEEVD (SEQ ID NO: |
| | | | GAACAACATCAAACTCTATGTCCG | | 70) |
| | | | CCGTGTGTTCATCATGGACAGC | | |
| | | | TGTGATGAGTTGATACCAGAGTAT | | |
| | | | CTCAATTTTATCCGTGGTGTGGTTG | | |
| | | | ACTCTGAGGATCTGCCCCTGA | | |
| | | | ACATCTCCCGAGAAATGCTCCAGC | | |
| | | | AGAGCAAAATCTTGAAAGTCATTC | | |
| | | | GCAAAAACATTGTTAAGAAGTG | | |
| | | | CCTTGAGCTCTTCTCTGAGCTGGCA | | |
| | | | GAAGACAAGGAGAATTACAAGAA | | |
| | | | ATTCTATGAGGCATTCTCTAAA | | |
| | | | AATCTCAAGCTTGGAATCCACGAA | | |
| | | | GACTCCACTAACCGCCGCCGCCTG | | |
| | | | TCTGAGCTGCTGCGCTATCATA | | |
| | | | CCTCCCAGTCTGGAGATGAGATGA | | |
| | | | CATCTCTGTCAGAGTATGTTTCTCG | | |
| | | | CATGAAGGAGACACAGAAGTC | | |
| | | | CATCTATTACATCACTGGTGAGAG | | |
| | | | CAAAGAGCAGGTGGCCAACTCAGC | | |
| | | | TTTTGTGCAGCGAGTGCGGAAA | | |
| | | | CGGGGCTTCGAGGTGGTATATATG | | |
| | | | ACCGAGCCCATTGACGAGTACTGT | | |
| | | | GTGCAGCAGCTCAAGGAATTTC | | |
| | | | ATGGGAAGAGCCTGGTCTCAGTTA | | |
| | | | CCAAGGAGGGTCTGGAGCTGCCTG | | |
| | | | AGGATGAGGAGGAGAAGAAGAA | | |
| | | | GATGGAAGAGAGCAAGGCAAAGT | | |
| | | | TTGAGAACCTCTGCAAGCTCATGA | | |
| | | | AAGAAATCTTAGATAAGAAGGTT | | |
| | | | GAGAAGGTGACAATCTCCAATAGA | | |
| | | | CTTGTGTCTTCACCTTGCTGCATTG | | |
| | | | TGACCAGCACCTACGGCTGGA | | |
| | | | CAGCCAATATGGAGCGGATCATGA | | |
| | | | AAGCCCAGGCACTTCGGGACAACT | | |
| | | | CCACCATGGGCTATATGATGGC | | |
| | | | CAAAAAGCACCTGGAGATCAACCC | | |
| | | | TGACCACCCCATTGTGGAGACGCT | | |
| | | | GCGGCAGAAGGCTGAGGCCGAC | | |
| | | | AAGAATGATAAGCCAGTTAAGGAC | | |
| | | | CTGGTGGTGCTGCTGTTTGAAACC | | |
| | | | GCCCTGCTATCTTCTGGCTTTT | | |
| | | | CCCTTGAGGATCCCCAGACCCACT | | |
| | | | CCAACCGCATCTATCGCATGATCA | | |
| | | | AGCTAGGTCTAGGTATTGATGA | | |
| | | | AGATGAAGTGGCAGCAGAGGAAC | | |
| | | | CCAATGCTGCAGTTCCTGATGAGA | | |
| | | | TCCCCCCTCTCGAGGGCGATGAG | | |
| | | | GATGCGTCTCGCATGGAAGAAGTC | | |
| | | | GATTAGGTTAGGAGTTCATAGTTG | | |
| | | | GAAAACTTGTGCCCTTCTATAG | | |
| | | | TGTCCCCATGGGCTCCCACTGCAG | | |
| | | | CCTCGAGTGCCCCTGTCCCACCTG | | |
| | | | GCTCCCCCTGCTGGTGTCTACT | | |
| | | | CTTTTTTTCCCTCTCCTGTCCTTGTG | | |
| | | | TTGAAGGCAGTAAACTAAGGGTGT | | |
| | | | CAAGCCCCATTCCCTCTCTA | | |
| | | | CTCTTGACAGCAGGATTGGATGTT | | |
| | | | GTGTATTGTGGTTTATTTTATTTTC | | |
| | | | TTCATTTTGTTCTGAAATTAA | | |
| | | | AGTATGCAAAATAAAGAATATGCC | | |
| | | | GTTTTTATACA (SEQ ID NO: 69) | | |
| | | NM_001271970.1 | AGAGGGGGGTCCCCCCCGCAGGTA | NP_001258899.1 | MPEEVHHIGEEEVETFAF |
| | | | CTCCACTCTCAGTCTGCAAAAGTG | | QAFIAQLMSLIINTFYSN |
| | | | TACGCCCGCAGAGCCGCCCCAG | | KEIFLRELI |
| | | | GTGCCTGGGTGTTGTGTGATTGAC | | SNASDALDKIRYESLTD |
| | | | CCGGGGAAGGAGGGGTCAGCCGA | | PSKLDSGKELKIDIIPNP |
| | | | TCCCTCCCCAACCCTCCATCCCA | | QERTLTLVDTGIGMTKA |
| | | | TCCCTGAGGATTGGGCTGGTACCC | | DLINNL |
| | | | GCGTCTCTCGGACAGATGCCTGAG | | GTIAKSGTKAFMEALQA |
| | | | GAAGTGCACCATGGAGAGGAGG | | GADISMIGQFGVGFYSA |
| | | | AGGTGGAGACTTTTGCCTTTCAGG | | YLVAEKVVVITKHNDD |
| | | | CAGAAATTGCCCAACTCATGTCCC | | EQYAWESS |
| | | | TCATCATCAATACCTTCTATTC | | AGGSFTVRADHGEPIGR |
| | | | CAACAAGGAGATTTTCCTTCGGGA | | GTKVILHLKEDQTEYLE |
| | | | GTTGATCTCTAATGCTTCTGATGCC | | ERRVKEVVKKHSQFIGY |
| | | | TTGGACAAGATTCGCTATGAG | | PITLYLE |
| | | | AGCCTGACAGACCCTTCGAAGTTG | | KEREKEISDDEAEEEKG |
| | | | GACAGTGGTAAAGAGCTGAAAATT | | EKEEEDKDDEEKPKIED |
| | | | GACATCATCCCCAACCCTCAGG | | VGSDEEDDSCKDKKKK |
| | | | AACGTACCCTGACTTTGGTAGACA | | TKKIKEKY |
| | | | CAGGCATTGGCATGACCAAAGCTG | | IDQEELNKTKPIWTRNP |
| | | | ATCTCATAAATAATTTGGGAAC | | DDITQEEYGEFYKSLTN |
| | | | CATTGCCAAGTCTGGTACTAAAGC | | DWEDHLAVKHFSVEGQ |
| | | | ATTCATGGAGGCTCTTCAGGCTGG | | LEFRALLF |
| | | | TGCAGACATCTCCATGATTGGG | | IPRRAPFDLFENKKKKN |
| | | | CAGTTTGGTGTTGGCTTTTATTCTG | | NIKLYVRRVFIMDSCDE |
| | | | CCTACTTGGTGGCAGAGAAAGTGG | | LIPEYLNFIRGVVDSEDL |
| | | | TTGTGATCACAAAGCACAACG | | PLNISR |
| | | | ATGATGAACAGTATGCTTGGGAGT | | EMLQQSKILKVIRKNIV |
| | | | CTTCTGCTGGAGGTTCCTTCACTGT | | KKCLELFSELAEDKENY |
| | | | GCGTGCTGACCATGGTGAGCC | | KKFYEAFSKNLKLGIHE |
| | | | CATTGGCAGGGGTACCAAAGTGAT | | DSTNRRR |
| | | | CCTCCATCTTAAAGAAGATCAGAC | | LSELLRYHTSQSGDEMT |
| | | | AGAGTACCTAGAAGAGAGGCGG | | SLSEYVSRMKETQKSIY |
| | | | GTCAAAGAAGTAGTGAAGAAGCAT | | YITGESKEQVANSAFVE |
| | | | TCTCAGTTCATAGGCTATCCCATCA | | RVRKRGF |
| | | | CCCTTTATTTGGAGAAGGAAC | | EVVYMTEPIDEYCVQQL |
| | | | GAGAGAAGGAAATTAGTGATGATG | | KEFDGKSLVSVTKEGLE |
| | | | AGGCAGAGGAAGAGAAAGGTGAG | | LPEDEEEKKKMEESKA |
| | | | AAAGAAGAGGAAGATAAAGATGA | | KFENLCKL |
| | | | TGAAGAAAAACCCAAGATCGAAG | | MKEILDKKVEKVTISNR |
| | | | ATGTGGGTTCAGATGAGGAGGATG | | LVSSPCCIVTSTYGWTA |
| | | | ACAGCGGTAAGGATAAGAAGAAG | | NMERIMKAQALRDNST |
| | | | AAAACTAAGAAGATCAAAGAGAA | | MGYMMAKK |
| | | | ATACATTGATCAGGAAGAACTAAA | | HLEINPDHPIVETLRQKA |
| | | | CAAGACCAAGCCTATTTGGACCA | | EADKNDKAVKDLVVLL |
| | | | GAAACCCTGATGACATCACCCAAG | | FETALLSSGFSLEDPQTH |
| | | | AGGAGTATGGAGAATTCTACAAGA | | SNRIYR |
| | | | GCCTCACTAATGACTGGGAAGA | | MIKLGLGIDEDEVAAEE |
| | | | CCACTTGGCAGTCAAGCACTTTTCT | | PNAAVPDEIPPLEGDED |
| | | | GTAGAAGGTCAGTTGGAATTCAGG | | ASRMEEVD (SEQ ID NO |
| | | | GCATTGCTATTTATTCCTCGT | | 72) |
| | | | CGGGCTCCCTTTGACCTTTTTGAGA | | |
| | | | ACAAGAAGAAAAAGAACAACATC | | |
| | | | AAACTCTATGTCCGCCGTGTGT | | |
| | | | TCATCATGGACAGCTGTGATGAGT | | |
| | | | TGATACCACAGTATCTCAATTTTAT | | |
| | | | CCGTGGTGTGGTTGACTCTGA | | |
| | | | GGATCTGCCCCTGAACATCTCCCG | | |
| | | | AGAAATGCTCCAGCAGAGCAAAAT | | |
| | | | CTTGAAAGTCATTCGCAAAAAC | | |
| | | | ATTGTTAAGAAGTGCCTTGAGCTC | | |
| | | | TTCTCTGAGCTGGCAGAAGACAAG | | |
| | | | GAGAATTACAAGAAATTCTATG | | |
| | | | AGGCATTCTCTAAAAATCTCAAGC | | |
| | | | TTGGAATCCACGAAGACTCCACTA | | |
| | | | ACCGCCGCCGCCTGTCTGAGCT | | |
| | | | GCTGCGCTATCATACCTCCCAGTCT | | |
| | | | GGAGATGAGATGACATCTCTGTCA | | |
| | | | GAGTATGTTTCTCGCATGAAG | | |
| | | | GAGACACAGAAGTCCATCTATTAC | | |
| | | | ATCACTGGTGAGAGCAAAGAGCAG | | |
| | | | GTGGCCAACTCAGCTTTTGTGG | | |
| | | | AGCGAGTGCGGAAACGGGGCTTCG | | |
| | | | AGGTGGTATATATGACCGAGCCCA | | |
| | | | TTGACGAGTACTGTGTGCAGCA | | |
| | | | GCTCAAGGAATTTGATGGGAAGAG | | |
| | | | CCTGGTCTCAGTTACCAAGGAGGG | | |
| | | | TCTGGAGCTGCCTGAGGATGAG | | |
| | | | GAGGAGAAGAAGAAGATGGAAGA | | |
| | | | GAGCAAGGCAAAGTTTGAGAACCT | | |
| | | | CTGCAAGCTCATGAAAGAAATCT | | |
| | | | TAGATAAGAAGGTTGAGAAGGTGA | | |
| | | | CAATCTCCAATACACTTGTGTCTTC | | |
| | | | ACCTTGCTGCATTGTGACCAG | | |
| | | | CACCTACGGCTGGACAGCCAATAT | | |
| | | | GGAGCGGATCATGAAAGCCCAGG | | |
| | | | CACTTCGGGACAACTCCACCATG | | |
| | | | CGCTATATGATGGCCAAAAAGCAC | | |
| | | | CTGGAGATCAACCCTGACCACCCC | | |
| | | | ATTGTGGAGACGCTGCGGCAGA | | |
| | | | AGGCTGAGGCCGACAAGAATGATA | | |
| | | | AGGCAGTTAACGACCTGGTGGTGC | | |
| | | | TGCTGTTTGAAACCCCCCTGCT | | |
| | | | ATCTTCTGGCTTTTCCCTTGAGGAT | | |
| | | | CCCCAGACCCACTCCAACCGCATC | | |
| | | | TATCGCATGATCAAGCTAGGT | | |
| | | | CTAGGTATTGATGAAGATGAAGTG | | |
| | | | CCAGCAGAGGAACCCAATGCTGCA | | |
| | | | GTTCCTGATGAGATCCCCCCTC | | |
| | | | TCGAGGGCGATGAGGATGCGTCTC | | |
| | | | CCATGGAAGAAGTGGATTAGGTTA | | |
| | | | GGAGTTCATAGTTGGAAAACTT | | |
| | | | GTGCCCTTGTATAGTGTCCCCATGG | | |
| | | | GCTCCCACTGCAGCCTCGAGTGCC | | |
| | | | CCTGTCCCACCTGGCTCCCCC | | |
| | | | TGCTGGTGTCTAGTGTTTTTTTCCC | | |
| | | | TCTCCTGTCCTTGTGTTGAAGGCAG | | |
| | | | TAAACTAAGGGTGTCAAGCC | | |
| | | | CCATTCCCTCTCTACTCTTGACAGC | | |
| | | | AGGATTGGATGTTGTGTATTGTGG | | |
| | | | TTTATTTTATTTTCTTCATTT | | |
| | | | TGTTCTGAAATTAAAGTATGCAAA | | |
| | | | ATAAAGAATATGCCGTTTTTATAC | | |
| | | | AGTTCT (SEQ ID NO: 71) | | |
| | | NM_001271971.1 | TTTTTCGGACCATGACGTCAAGGT | NP_001258900.1 | MPEEVHHGEEEVETFAF |
| | | | GGGCTGGTGGCGCCAGGTGCGGGG | | QAEIAQLMSLIINTFYSN |
| | | | TTGACAATCATACTCCTTTAAG | | KEIFLRELI |
| | | | GCGGAGGGATCTACAGGAGGGCG | | SNASDALDKIRYESLTD |
| | | | GCTGTACTGTGCTTCGCCTTATATA | | PSKLDSGKELKIDISMIG |
| | | | GGGCGACTTGGGGCACGCAGTA | | QFGVGFYSAYLVAEKV |
| | | | GCTCTCTCGAGTCACTCCGGCGCA | | VVITKHN |
| | | | GTGTTGGGACTGTCTGGGTATCGG | | DDEQYAWESSAGGSFT |
| | | | AAAGCAAGCCTACGTTGCTCAC | | VRADHGEPIGRGTKVIL |
| | | | TATTACGTATAATCCTTTTCTTTTC | | HLKEDQTEYLEERRVKE |
| | | | AAGATGCCTGAGGAAGTGCACCAT | | VVKKHSQF |
| | | | GGAGAGGAGGAGGTGGAGACT | | IGYPITLYLEKEREKEIS |
| | | | TTTGCCTTTCAGGCAGAAATTGCCC | | DDEAEEEKGEKEEEDK |
| | | | AACTCATGTCCCTCATCATCAATA | | DDEEKPKIEDVGSDEED |
| | | | CCTTCTATTCCAACAAGGAGA | | DSGKDKK |
| | | | TTTTCCTTCGGGAGTTGATCTCTAA | | KKIKKIKEKYIDQEELN |
| | | | TGCTTCTGATGCCTTGGACAAGATT | | KIKPIWTRNPDDITQEE |
| | | | CGCTATGAGAGCCTGACAGA | | YGEFYKSLINDWEDHL |
| | | | CCCTTCGAAGTTGGACAGTGGTAA | | AVKHFSVE |
| | | | AGAGCTGAAAATTGACATCTCCAT | | GQLEFRALLFIPRRAPFD |
| | | | GATTGGGCAGTTTGGTGTTGGC | | LFENKKKKNNIKLYVRR |
| | | | TTTTATTCTGCCTACTTGGTGGCAG | | VFIMDSCDELIPEYLNFI |
| | | | AGAAAGTGGTTGTGATCACAAAGC | | RGVVD |
| | | | ACAACGATGATGAACAGTATG | | SEDLPLNISREMLQQSKI |
| | | | CTTGGGAGTCTTCTGCTGGAGGTTC | | LKVIRKNIVKKCLELFSE |
| | | | CTTCACTGTGCGTGCTGACCATGGT | | LAEDKENYKKFYEAFS |
| | | | GAGCCCATTGGCAGGGGTAC | | KNLKLG |
| | | | CAAAGTGATCCTCCATCTTAAAGA | | IHEDSTNRRRLSELLRY |
| | | | AGATCAGACAGAGTACCTAGAAGA | | HTSQSGDEMISLSEYVS |
| | | | GAGGCGGGTCAAAGAAGTAGTG | | RMKETQKSIYYITGESK |
| | | | AAGAAGCATTCTCAGTTCATAGGC | | EQVANSA |
| | | | TATCCCATCACGCTTTATTTGGAGA | | FVERVRKRGFEVVYMT |
| | | | AGGAACGAGAGAAGGAAATTA | | EPIDEYCVQQLKEFDGK |
| | | | GTGATGATGAGGCAGAGGAAGAG | | SLVSVTKEGLELPEDEE |
| | | | AAAGGTGAGAAAGAAGAGGAAGA | | EKKKMEES |
| | | | TAAAGATGATGAAGAAAAACCCA | | KAKFENLCKLMKEILDK |
| | | | A | | KVEKVTISNRLVSSPCCI |
| | | | CATCGAAGATGTGGGTTCAGATGA | | VTSTYGWTANMERIMK |
| | | | GGAGGATGACAGCGGTAAGGATA | | AQALRDN |
| | | | AGAAGAAGAAAACTAAGAAGATC | | STMGYMMAKKHLEINP |
| | | | AAAGAGAAATACATTGATCAGGAA | | DHPIVETLRQKAEADKN |
| | | | GAACTAAACAAGACCAAGCCTATT | | DKAVKDLVVLLFETAL |
| | | | TGGACCAGAAACCGTGATGACA | | LSSGFSLED |
| | | | TCACCCAAGAGGAGTATGGAGAAT | | PQTHSNRIYRMIKLGLGI |
| | | | TCTACAAGAGCCTCACTAATGACT | | DEDEVAAEEPNAAVPD |
| | | | GGGAAGACCACTTGGCAGTCAA | | EIPPLEGDEDASRMEEV |
| | | | GCACTTTTCTGTAGAAGGTCAGTT | | D (SEQ ID NO: 74) |
| | | | GGAATTCAGGGCATTGCTATTTATT | | |
| | | | CCTCGTCGGGCTCCCTTTGAC | | |
| | | | CTTTTTGAGAACAAGAAGAAAAAG | | |
| | | | AACAACATCAAACTCTATGTCCGC | | |
| | | | CGTGTGTTCATCATGGACAGCT | | |
| | | | GTGATGAGTTGATACCAGAGTATC | | |
| | | | TCAATTTTATCCGTGGTGTGGTTGA | | |
| | | | CTCTGAGGATCTGCCCCTGAA | | |
| | | | CATCTCCCGAGAAATGCTCCAGCA | | |
| | | | GAGCAAAATCTTGAAAGTCATTCG | | |
| | | | CAAAAACATTGTTAAGAAGTCC | | |
| | | | CTTGAGCTCTTCTCTGAGCTGGCAG | | |
| | | | AAGACAAGGAGAATTACAAGAAA | | |
| | | | TTCTATGAGGCATTCTCTAAAA | | |
| | | | ATCTCAAGCTTGGAATCCACGAAG | | |
| | | | ACTCCACTAACCGCCGCCGCCTGT | | |
| | | | CTGAGCTGCTGCGCTATCATAC | | |
| | | | CTCCCAGTCTGGAGATGAGATGAC | | |
| | | | ATCTCTGTCAGAGTATGTTTCTCGC | | |
| | | | ATGAAGGAGACACAGAAGTCC | | |
| | | | ATCTATTACATCACTGGTGAGAGC | | |
| | | | AAAGAGCAGGTGGCCAACTCAGCT | | |
| | | | TTTGTGGAGCCAGTGCGGAAAC | | |
| | | | GGGGCTTCGAGGTGGTATATATGA | | |
| | | | CCGAGCCCATTGACGAGTACTGTG | | |
| | | | TGCAGCAGCTCAAGGAATTTGA | | |
| | | | TGGGAAGAGCCTGGTCTCAGTTAC | | |
| | | | CAAGGAGGGTCTGGAGCTGCCTGA | | |
| | | | GGATGAGGAGGAGAAGAAGAAC | | |
| | | | ATGGAAGAGAGCAAGGCAAAGTTT | | |
| | | | GAGAACCTCTGCAAGCTCATGAAA | | |
| | | | GAAATCTTAGATAAGAAGGTTG | | |
| | | | AGAAGGTGACAATCTCCAATAGAC | | |
| | | | TTGTGTCTTCACCTTGCTGCATTGT | | |
| | | | GACCAGCACCTACGGCTGGAC | | |
| | | | AGCCAATATGGAGCGGATCATGAA | | |
| | | | AGCCCAGGCACTTCGGGACAACTC | | |
| | | | CACCATGGGCTATATGATGGCC | | |
| | | | AAAAAGCACCTGGAGATCAACCCT | | |
| | | | GACCACCCCATTGTGGAGACGCTG | | |
| | | | CGGCAGAAGGCTGAGGCCGACA | | |
| | | | AGAATGATAAGGCAGTTAAGGACC | | |
| | | | TGGTGGTGCTGCTGTTTGAAACCG | | |
| | | | CCCTGCTATCTTCTGGCTTTTC | | |
| | | | CCTTGAGGATCCCCAGACCCACTC | | |
| | | | CAACCGCATCTATCGCATGATCAA | | |
| | | | GCTAGGTCTAGGTATTCATGAA | | |
| | | | GATGAAGTGGCAGCAGAGGAACC | | |
| | | | CAATGCTGCAGTTCCTGATGAGAT | | |
| | | | CCCCCCTCTCGAGGGCGATGAGG | | |
| | | | ATGCGTCTCGCATGGAAGAAGTCG | | |
| | | | ATTAGGTTAGGACTTCATAGTTGG | | |
| | | | AAAACTTGTGCCCTTGTATACT | | |
| | | | GTCCCCATGGGCTCCCACTGCAGC | | |
| | | | CTCGAGTGCCCCTGTCCCACCTGG | | |
| | | | CTCCCCCTGCTGGTGTCTAGTG | | |
| | | | TTTTTTTCCCTCTCCTGTCCTTGTGT | | |
| | | | TGAAGGCAGTAAACTAAGGGTGTC | | |
| | | | AAGCCCCATTCCCTCTCTAC | | |
| | | | TCTTGACAGCAGGATTGGATGTTG | | |
| | | | TGTATTGTGGTTTATTTTATTTTCTT | | |
| | | | CATTTTGTTCTGAAATTAAA | | |
| | | | GTATGCAAAATAAAGAATATGCCG | | |
| | | | TTTTTATACAGTTCT (SEQ ID NO: | | |
| | | | 73) | | |
| | | NM_001271972.1 | TTTTTCGGACCATGACGTCAAGGT | NP_001258901.1 | MPEEVHHGEEEVETFAF |
| | | | GGGCTGGTGGCGCCAGGTGCGGGG | | QAEIAQLMSLIINTFYSN |
| | | | TTGACAATCATACTCCTTTAAG | | KEIFLRELI |
| | | | GCGGAGGGATCTACAGGAGGGCG | | SNASDALDKIRYESLTD |
| | | | GCTGTACTGTGCTTCGCCTTATATA | | PSKLDSGKELKIDIPNP |
| | | | GGGCGACTTGGGGCACGCAGTA | | QERTLTLVDTGIGMTKA |
| | | | CCTCTCTCGAGTCACTCCGGCCCA | | DLINNL |
| | | | GTGTTGGGACTGTCTGGGTATCGG | | GTIAKSGTKAFMEALQF |
| | | | AAAGCAAGCCTACGTTGCTCAC | | GVGFYSAYLVAEKVVV |
| | | | TATTACGTATAATCCTTTTCTTTTC | | ITKHNDDEQYAWESSA |
| | | | AAGATGCCTGAGGAAGTGCACCAT | | GGSFTVRAD |
| | | | GGAGAGGAGGAGGTGGAGACT | | HGEPIGRGTKVILHLKE |
| | | | TTTGCCTTTCAGGCAGAAATTGCCC | | DQTEYLEERRVKEVVK |
| | | | AACTCATGTCCCTCATCATCAATA | | KHSQFIGYPITLYLEKER |
| | | | CCTTCTATTCCAACAAGGAGA | | EKEISDD |
| | | | TTTTCCTTCGGGAGTTGATCTCTAA | | EAEEEKGEKEEEDKDDE |
| | | | TGCTTCTGATGCCTTGGACAAGATT | | EKPKIEDVGSDEEDDSG |
| | | | CGCTATGAGAGCCTGACAGA | | KDKKKKTKKIKEKYIDQ |
| | | | CCCTTCGAAGTTGGACAGTGGTAA | | EELNKTK |
| | | | AGAGCTGAAAATTGACATCATCCC | | PIWTRNPDDITQEEYGE |
| | | | CAACCCTCAGGAACGTACCCTG | | FYKSLINDWEDHLAVK |
| | | | ACTTTGGTAGACACAGGCATTGGC | | HFSVEGQLEFRALLFIPR |
| | | | ATGACCAAAGCTGATCTCATAAAT | | RAPFDLF |
| | | | AATTTGGGAACCATTGCCAAGT | | ENKKKKNNIKLYVRRV |
| | | | CTGGTACTAAAGCATTCATGGAGG | | FIMDSCDELIPEYLNFIR |
| | | | CTCTTCAGTTTGGTGTTGGCTTTTA | | GVVDSEDLPLNISREML |
| | | | TTCTGCCTACTTGGTGGCAGA | | QQSKILK |
| | | | GAAAGTGGTTGTGATCACAAAGCA | | VIRKNIVKKCLELFSELA |
| | | | CAACGATGATGAACAGTATGCTTG | | EDKENYKKFYEAFSKIN |
| | | | GGAGTCTTCTGCTGGAGGTTCC | | LKLGIHEDSINRRRLSE |
| | | | TTCACTGTGCGTGCTGACCATGGT | | LLRYHTS |
| | | | GAGCCCATTGGCAGGGGTACCAAA | | QSGDEMTSLSEYVSRM |
| | | | GTGATCCTCCATCTTAAAGAAG | | KETQKSIYYITGESKEQ |
| | | | ATCAGACAGAGTACCTAGAAGAGA | | VANSAFVERVRKRGFE |
| | | | GGCGGGTCAAAGAAGTAGTGAAG | | VVYMTEPID |
| | | | AAGCATTCTCAGTTCATAGGCTA | | EYCVQQLKEFDGKSLV |
| | | | TCCCATCACCCTTTATTTGGAGAAG | | SVTKEGLELPEDEEEKK |
| | | | GAACGAGAGAAGGAAATTACTGA | | KMEESKAKFENLCKLM |
| | | | TTGATGAGGCAGAGGAAGAGAAA | | KEILDKKVE |
| | | | GGTGAGAAAGAAGAGGAAGATAA | | KVTISNRLVSSPCCIVTS |
| | | | AGATGATGAAGAAAAACCCAAGA | | TYGWTANMERIMKAQ |
| | | | TCGAAGATGTGGGTTCAGATGAGG | | ALRDNSTMGYMMAKK |
| | | | AGGATGACAGCGGTAAGGATAAG | | HLEINPDAPI |
| | | | AAGAAGAAAACTAAGAAGATCAA | | VETLRQKAEADKNDKA |
| | | | AGAGAAATACATTGATCAGGAAGA | | VKDLVVLLFETALLSSG |
| | | | ACTAAACAAGACCAAGCCTATTTG | | FSLEDPQTHSNRIYRMI |
| | | | GACCAGAAACCCTGATGACATCAC | | KLGLGIDE |
| | | | CCAAGAGGAGTATGGAGAATTC | | DEVAAEEPNAAVPDEIP |
| | | | TACAAGAGCCTCACTAATGACTGG | | PLEGDEDASRMEEVD |
| | | | GAAGACCACTTGGCAGTCAAGCAC | | (SEQ ID NO: 76) |
| | | | TTTTCTGTAGAAGGTCAGTTGG | | |
| | | | AATTCAGGGCATTGCTATTTATTCC | | |
| | | | TCGTCGGGCTCCCTTTGACCTTTTT | | |
| | | | GAGAACAAGAAGAAAAAGAA | | |
| | | | CAACATCAAACTCTATGTCCGCCG | | |
| | | | TGTGTTCATCATGGACAGCTGTGA | | |
| | | | TGAGTTGATACCAGAGTATCTC | | |
| | | | AATTTTATCCGTGGTGTGGTTGACT | | |
| | | | CTGAGGATCTGCCCCTGAACATCT | | |
| | | | CCCGAGAAATGCTCCAGCAGA | | |
| | | | GCAAAATCTTGAAAGTCATTCGCA | | |
| | | | AAAACATTGTTAAGAAGTGCCTTG | | |
| | | | AGCTCTTCTCTGAGCTGGCAGA | | |
| | | | AGACAACGAGAATTACAAGAAATT | | |
| | | | CTATGAGGCATTCTCTAAAAATCT | | |
| | | | CAAGCTTGGAATCCACGAAGAC | | |
| | | | TCCACTAACCGCCGCCGCCTGTCT | | |
| | | | GAGCTGCTGCGCTATCATACCTCC | | |
| | | | CAGTCTGGAGATGAGATGACAT | | |
| | | | CTCTGTCAGAGTATGTTTCTCGCAT | | |
| | | | GAAGGAGACACAGAAGTCCATCTA | | |
| | | | TTACATCACTGGTGAGAGCAA | | |
| | | | AGAGCAGGTGGCCAACTCAGCTTT | | |
| | | | TGTGGAGCGAGTGCGGAAACGGG | | |
| | | | GCTTCGAGGTGGTATATATGACC | | |
| | | | GAGCCCATTGACGAGTACTGTGTG | | |
| | | | CAGCAGCTCAAGGAATTTGATGGG | | |
| | | | AAGAGCCTGGTCTCAGTTACCA | | |
| | | | AGGAGGGTCTGGAGCTGCCTGAGG | | |
| | | | ATGAGGAGGAGAAGAAGAAGATG | | |
| | | | GAAGAGAGCAAGGCAAAGTTTGA | | |
| | | | GAACCTCTGCAAGCTCATGAAAGA | | |
| | | | AATCTTAGATAAGAAGGTTGAGAA | | |
| | | | GGTGACAATCTCCAATAGACTT | | |
| | | | GTGTCTTCACCTTGCTGCATTGTGA | | |
| | | | CCAGCACCTACGGCTGGACACCCA | | |
| | | | ATATGGAGCGGATCATGAAAG | | |
| | | | CCCAGGCACTTCGGGACAACTCCA | | |
| | | | CCATGGGCTATATGATGGCCAAAA | | |
| | | | AGCACCTGGAGATCAACCCTGA | | |
| | | | CCACCCCATTGTGGAGACGCTGCG | | |
| | | | GCAGAAGGCTGAGGCCGACAAGA | | |
| | | | ATGATAAGGCAGTTAAGGACCTG | | |
| | | | GTGGTGCTGCTGTTTGAAACCGCC | | |
| | | | CTGCTATCTTCTGGCTTTTCCCTTG | | |
| | | | AGGATCCCCAGACCCACTCCA | | |
| | | | ACCGCATCTATCGCATGATCAAGC | | |
| | | | TAGGTCTAGGTATTCATGAAGATG | | |
| | | | AAGTGGCAGCAGAGGAACCCAA | | |
| | | | TGCTGCAGTTCCTGATGAGATCCC | | |
| | | | CCCTCTCGAGGGCGATGAGGATGC | | |
| | | | GTCTCGCATGGAAGAAGTCGAT | | |
| | | | TAGGTTAGGAGTTCATAGTTGGAA | | |
| | | | AACTTGTGCCCTTGTATAGTGTCCC | | |
| | | | CATGGGCTCCCACTGCAGCCT | | |
| | | | CGAGTGCCCCTGTCCCACCTGGCT | | |
| | | | CCCCCTGCTGGTCTCTAGTGTTTTT | | |
| | | | TTCCCTCTCCTGTCCTTGTGT | | |
| | | | TGAAGGCAGTAAACTAAGGGTGTC | | |
| | | | AAGCCCCATTCCCTCTCTACTCTTG | | |
| | | | ACAGCAGGATTGGATGTTGTG | | |
| | | | TATTGTGGTTTATTTTATTTTCTTCA | | |
| | | | TTTTGTTCTGAAATTAAAGTATGCA | | |
| | | | AAATAAAGAATATGCCGTT | | |
| | | | TTTATACAGTTCT (SEQ ID NO: | | |
| | | | 75) | | |
| | | NM_001371238.1 | AGTGACGAGTGTCGGCCTGGTGGC | NP_001358367.1 | MPEEVHHIGEEEVETFAF |
| | | | TACGGCCACCATCTTTCTTGGGTTT | | QAFIAQLMSLIINTFYSN |
| | | | GGTCCTGTTCTGTAATTTTGT | | KEIFLRELI |
| | | | GCTGTGAAAGGGTCGTGGTGGAGC | | SNASDALDKIRYESLTD |
| | | | TTTTGGCTTAAGAATTCTTTGTCCG | | PSKLDSGKELKIDIIPNP |
| | | | GATTTAATTGCTCCTCCGATG | | |
| | | | CCTGAGGAAGTGCACCATGGAGAG | | QERTLTLVDTGIGMTKA |
| | | | GAGGAGGTGGAGACTTTTGCCTTT | | DLINNL |
| | | | CAGGCAGAAATTGCCCAACTCA | | GTIAKSGIKAFMEALQA |
| | | | TGTCCCTCATCATCAATACCTTCTA | | GADISMIGQFGVGFYSA |
| | | | TTCCAACAAGGAGATTTTCCTTCG | | YLVAEKVVVITKHNDD |
| | | | GGAGTTGATCTCTAATCCTTC | | EQYAWESS |
| | | | TGATGCCTTGGACAAGATTCGCTA | | AGGSFTVRADHCEPIGR |
| | | | TGAGAGCCTGACAGACCCTTCGAA | | GTKVILHLKEDQTEYLE |
| | | | GTTGGACAGTGGTAAAGAGCTG | | ERRVKEVVKKHSQFIGY |
| | | | AAAATTGACATCATCCCCAACCCT | | PITLYLE |
| | | | CAGGAACGTACCCTGACTTTGGTA | | KEREKEISDDEABEEKG |
| | | | GACACAGGCATTGGCATGACCA | | EKEEEDKDDEEKPKIED |
| | | | AAGCTGATCTCATAAATAATTTGG | | VGSDEEDDSGKDKKKK |
| | | | GAACCATTGCCAAGTCTGGTACTA | | TKKIKEKY |
| | | | AAGCATTCATGGAGGCTCTTCA | | IDQEELNKTKPIWTRNP |
| | | | GGCTGGTGCAGACATCTCCATGAT | | DDITQEEYGEFYKSLTN |
| | | | TGGGCAGTTTGGTGTTGGCTTTTAT | | DWEDHLAVKHFSVEGQ |
| | | | TCTGCCTACTTGGTGGCAGAG | | LEFRALLF |
| | | | AAAGTGGTTGTGATCACAAAGCAC | | IPRRAPFDLFENKKKKN |
| | | | AACGATGATGAACAGTATGCTTGG | | NIKLYVRRVFIMDSCDE |
| | | | GAGTCTTCTGCTGGAGGTTCCT | | LIPEYLNFIRGVVDSEDL |
| | | | TCACTGTGCGTGCTGACCATGGTG | | PLNISR |
| | | | AGCCCATTGGCAGGGGTACCAAAG | | EMLQQSKILKVIRKNIV |
| | | | TGATCCTCCATCTTAAAGAAGA | | KKCLELFSELAEDKENY |
| | | | TCAGACAGAGTACCTAGAAGAGAG | | KKFYEAFSKNLKLGHHE |
| | | | GCGGGTCAAAGAAGTACTGAAGA | | DSTNRRR |
| | | | AGCATTCTCAGTTCATAGGCTAT | | LSELLRYHTSQSGDEMT |
| | | | CCCATCACCCTTTATTTGGAGAAG | | SLSEYVSRMKETQKSIY |
| | | | GAACGAGAGAAGGAAATTAGTGA | | YITGESKEQVANSAFVE |
| | | | TGATGAGGCAGAGGAAGAGAAAG | | RVRKRGF |
| | | | GTGAGAAACAAGAGGAAGATAAA | | EVVYMTEPIDEYCVQQL |
| | | | GATGATGAAGAAAAACCCAAGATC | | KEFDGKSLVSVTKEGLE |
| | | | GAAGATGTGGGTTCAGATGAGGA | | LPEDEEEKKKMEESKA |
| | | | GGATGACAGCGGTAAGCATAAGA | | KFENICKL |
| | | | AGAAGAAAACTAAGAAGATCAAA | | MKEILDKKVEKVTISNR |
| | | | GAGAAATACATTGATCAGGAAGAA | | LVSSPCCIVTSTYGWTA |
| | | | CTAAACAAGACCAAGCCTATTTGG | | NMERIMKAQALRDNST |
| | | | ACCAGAAACCCTGATGACATCACC | | MGYMMAKK |
| | | | CAAGAGGAGTATGGAGAATTCT | | HLEINPDHPIVETLRQKA |
| | | | ACAAGAGCCTCACTAATGACTGGG | | EADKNDKAVKDLVVLL |
| | | | AAGACCACTTGGCAGTCAAGCACT | | FETALLSSGFSLEDPQTH |
| | | | TTTCTCTAGAAGGTCAGTTGGA | | SNRIYR |
| | | | ATTCAGGGCATTGCTATTTATTCCT | | MIKLGLGIDEDEVAAEE |
| | | | CGTCGGGCTCCCTTTGACCTTTTTG | | PNAAVPDEIPPLEGDED |
| | | | AGAACAAGAAGAAAAAGAAC | | ASRMEEVD (SEQ ID NO: |
| | | | AACATCAAACTCTATGTCCGCCGT | | 78) |
| | | | GTGTTCATCATGGACAGCTGTCAT | | |
| | | | CAGTTGATACCAGAGTATCTCA | | |
| | | | ATTTTATCCGTGGTGTGGTTGACTC | | |
| | | | TGAGGATCTGCCCCTGAACATCTC | | |
| | | | CCGAGAAATGCTCCAGCAGAG | | |
| | | | CAAAATCTTGAAAGTCATTCGCAA | | |
| | | | AAACATTGTTAAGAAGTGCCTTGA | | |
| | | | GCTCTTCTCTGAGCTGGCAGAA | | |
| | | | GACAAGGAGAATTACAAGAAATTC | | |
| | | | TATGAGGCATTCTCTAAAAATCTC | | |
| | | | AAGCTTGGAATCCACGAAGACT | | |
| | | | CCACTAACCGCCGCCGCCTGTCTG | | |
| | | | AGCTGCTGCGCTATCATACCTCCC | | |
| | | | AGTCTGGAGATGAGATGACATC | | |
| | | | TCTGTCAGAGTATGTTTCTCGCATG | | |
| | | | AAGGAGACACAGAAGTCCATCTAT | | |
| | | | TACATCACTGGTGAGAGCAAA | | |
| | | | CAGCAGGTGGCCAACTCAGCTTTT | | |
| | | | GTGGAGCGAGTGCGGAAACGGGG | | |
| | | | CTTCGAGGTGGTATATATGACCG | | |
| | | | AGCCCATTGACGAGTACTGTGTCC | | |
| | | | AGCAGCTCAAGGAATTTGATGGGA | | |
| | | | AGAGCCTGGTCTCAGTTACCAA | | |
| | | | GGAGGGTCTGGAGCTGCCTGAGGA | | |
| | | | TGAGGAGGAGAAGAAGAAGATGG | | |
| | | | AAGAGAGCAAGGCAAAGTTTGAG | | |
| | | | AACCTCTGCAAGCTCATGAAAGAA | | |
| | | | ATCTTAGATAAGAAGGTTGAGAAG | | |
| | | | CTGACAATCTCCAATAGACTTG | | |
| | | | TGTCTTCACCTTGCTGCATTGTGAC | | |
| | | | CAGCACCTACGGCTGGACAGCCAA | | |
| | | | TATGGAGCGGATCATGAAAGC | | |
| | | | CCAGGCACTTCGGGACAACTCCAC | | |
| | | | CATGGGCTATATCATGGCCAAAAA | | |
| | | | GCACCTGGAGATCAACCCTGAC | | |
| | | | CACCCCATTGTGGAGACGCTGCGG | | |
| | | | CAGAAGGCTGAGGCCGACAAGAA | | |
| | | | TGATAAGGCACTTAAGGACCTGG | | |
| | | | TGGTGCTGCTGTTTGAAACCCCCCT | | |
| | | | GCTATCTTCTGGCTTTTCCCTTGAG | | |
| | | | CATCCCCAGACCCACTCCAA | | |
| | | | CCGCATCTATCGCATGATCAAGCT | | |
| | | | AGGTCTAGGTATTGATGAAGATGA | | |
| | | | AGTGGCACCAGAGGAACCCAAT | | |
| | | | GCTGCAGTTCCTGATGAGATCCCC | | |
| | | | CCTCTCGAGGGCGATGAGGATGCG | | |
| | | | TCTCGCATGGAAGAAGTCGATT | | |
| | | | AGGTTAGGAGTTCATAGTTGGAAA | | |
| | | | ACTTGTGCCCTTGTATAGTGTCCCC | | |
| | | | ATGGGCTCCCACTGCAGCCTC | | |
| | | | GAGTGCCCCTGTCCCACCTGGCTC | | |
| | | | CCCCTGCTGGTGTCTAGTGTTTTTT | | |
| | | | TCCCTCTCCTGTCCTTGTGTT | | |
| | | | GAAGGCAGTAAACTAAGGGTGTCA | | |
| | | | AGCCCCATTCCCTCTCTACTCTTGA | | |
| | | | CAGCAGGATTGGATGTTGTGT | | |
| | | | ATTGTGGTTTATTTTATTTTCTTCAT | | |
| | | | TTTGTTCTGAAATTAAAGTATGCA | | |
| | | | AAATAAAGAATATGCCGTTT | | |
| | | | TTATACA (SEQ ID NO: 72) | | |

In some embodiments, the disclosure provides a composition comprising nucleic acid sequences complementary to one or a combination of: INFAIP6, S100A8, TNFSF10, DRAM1, LY96, QPCT, KYNU, ENTPD1, CLIC1, ATP6V0E1, HSP90AB1, NCL, and CIRBP. In some embodiments, the disclosure provides a composition comprising nucleic acid sequences complementary to all of the 13 biomarkers and/or antibodies or antibody fragments that have strong affinity to disclosed herein. In some embodiments, the biomarker INFAIP6, as used herein, refers to a nucleic acid comprising at least about 70%, 75%, 80%, 85%, 90%, 91%, 92%, 93%, 94%, 95%, 96%, 97%, 98%, 99% or 100% sequence identity to SEQ ID NO: 1, or a functional fragment or variant thereof, or a nucleic acid encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 2, or a functional fragment or variant thereof. In some embodiments, the biomarker S100A8, as used herein, refers to a nucleic acid comprising at least about 70%, 75%, 80%, 85%, 90%, 91%, 92%, 93%, 94%, 95%, 96%, 97%, 98%, 99% or 100% sequence identity to SEQ ID NO: 3, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 4, or a functional fragment or variant thereof. In some embodiments, the biomarker S100A8, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 5, or a functional fragment or variant thereof, or a nucleic acid encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 6, or a functional fragment or variant thereof. In some embodiments, the biomarker S100A8, as used herein, refers to a nucleic acid comprising at least about 70%, 75%, 80%, 85%, 90%, 91%, 92%, 93%, 94%, 95%, 96%, 97%, 98%, 99% or 100% sequence identity to SEQ ID NO: 7, or a functional fragment or variant thereof, or a nucleic acid encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 8, or a functional fragment or variant thereof. In some embodiments, the biomarker S100A8, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 9, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 10, or a functional fragment or variant thereof. In some embodiments, the biomarker S100A8, as used herein, refers to a nucleic acid comprising at least about 70%, 75%, 80%, 85%, 90%, 91%, 92%, 93%, 94%, 95%, 96%, 97%, 98%, 99% or 100% sequence identity to SEQ ID NO: 11, or a functional fragment or variant thereof, or a nucleic acid encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 12, or a functional fragment or variant thereof. In some embodiments, the biomarker DRAM1, as used herein, refers to a nucleic acid comprising at least about 70%, 75%, 80%, 85%, 90%, 91%, 92%, 93%, 94%, 95%, 96%, 97%, 98%, 99% or 100% sequence identity to SEQ ID NO: 13, or a functional fragment or variant thereof, or a nucleic acid encoding a polypeptide comprising at least about 70%, 75%, 80%, 85%, 90%, 91%, 92%, 93%, 94%, 95%, 96%, 97%, 98%, 99% or 100% sequence identity to SEQ ID NO: 14, or a functional fragment or variant thereof. In some embodiments, the biomarker TNFSF10, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 15, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 16, or a functional fragment or variant thereof. In some embodiments, the biomarker TNFSF10, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 17, or a functional fragment or variant thereof or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 18, or a functional fragment or variant thereof. In some embodiments, the biomarker INFSF10, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 19, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 20, or a functional fragment or variant thereof. In some embodiments, the biomarker LY96, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 21, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 22, or a functional fragment or variant thereof. In some embodiments, the biomarker LY96, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 23, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 24, or a functional fragment or variant thereof. In some embodiments, the biomarker QPCT, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 25, or a functional fragment or variant thereof, or a nucleic acid encoding a polypeptide comprising at least about 70%, 75%, 80%, 85, 900%, 91%, 92%, 93%, 94%, 95%, 96%, 97%, 98%, 99% or 100% sequence identity to SEQ ID NO: 26, or a functional fragment or variant thereof. In some embodiments, the biomarker KYNU, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 27, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 28, or a functional fragment or variant thereof. In some embodiments, the biomarker KYNU, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 29, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 30, or a functional fragment or variant thereof. In some embodiments, the biomarker KYNU, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 31, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99%/or 100% sequence identity to SEQ ID NO: 32, or a functional fragment or variant thereof. In some embodiments, the biomarker ENTPD1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 33, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 34, or a functional fragment or variant thereof. In some embodiments, the biomarker ENTPDJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 35, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 36, or a functional fragment or variant thereof. In some embodiments, the biomarker ENTPD1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 37, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 38, or a functional fragment or variant thereof. In some embodiments, the biomarker ENTPDJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 39, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 40, or a functional fragment or variant thereof. In some embodiments, the biomarker ENTPD1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 41, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 42, or a functional fragment or variant thereof. In some embodiments, the biomarker ENTPDJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 43, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 44, or a functional fragment or variant thereof. In some embodiments, the biomarker ENTPDJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 45, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 46, or a functional fragment or variant thereof. In some embodiments, the biomarker ENTPDJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 47, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 48, or a functional fragment or variant thereof. In some embodiments, the biomarker ENTPD1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 49, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 50, or a functional fragment or variant thereof. In some embodiments, the biomarker CLIC1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 51, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 52, or a functional fragment or variant thereof. In some embodiments, the biomarker CLIC1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 53, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 54, or a functional fragment or variant thereof. In some embodiments, the biomarker CLIC1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 55, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 56, or a functional fragment or variant thereof. In some embodiments, the biomarker ATP6V0E1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 57, or a functional fragment or variant thereof or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 58, or a functional fragment or variant thereof. In some embodiments, the biomarker NCL, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 59, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 60, or a functional fragment or variant thereof. In some embodiments, the biomarker CIRBP, as used herein, refers to a nucleic acid comprising at least about 70%, 75%, 80%, 85%, 90%, 91%, 92%, 93%, 94%, 95%, 96%, 97%, 98%, 99% or 100% sequence identity to SEQ ID NO: 61, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 62, or a functional fragment or variant thereof. In some embodiments, the biomarker CIRBP, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 63, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 64, or a functional fragment or variant thereof. In some embodiments, the biomarker CIRBP, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 65, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 66, or a functional fragment or variant thereof. In some embodiments, the biomarker HSP90ABJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 67, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 68, or a functional fragment or variant thereof. In some embodiments, the biomarker HSP90ABJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 69, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 70, or a functional fragment or variant thereof. In some embodiments, the biomarker HSP90AB1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 71, or a functional fragment or variant thereof or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 72, or a functional fragment or variant thereof. In some embodiments, the biomarker HSP90ABJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 73, or a functional fragment or variant thereof or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 74, or a functional fragment or variant thereof. In some embodiments, the biomarker HSP90ABJ, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 75, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 76, or a functional fragment or variant thereof. In some embodiments, the biomarker HSP90AB1, as used herein, refers to a nucleic acid comprising at least about 70%, 75%, 80%, 85, 90%, 91%, 92%, 93%, 94%, 95%, 96%, 97%, 98%, 99% or 100% sequence identity to SEQ ID NO: 77, or a functional fragment or variant thereof, or a nucleic acid encoding a polypeptide comprising at least about 70%, 75%, 80%, 85, 90%, 91%, 92%, 93%, 94%, 95%, 96%, 97%, 98%, 99% or 100% sequence identity to SEQ ID NO: 78, or a functional fragment or variant thereof.

As used herein, the term “variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule or amino acid sequence of the disclosure will have at least about 70%, 75%, 80%, 85%, 87%, 90%, 91%, 92%, 93%, 94%, 95%, 96%, 97%, 98%, 99% or more sequence identity to that particular polynucleotide as determined by sequence alignment programs and parameters as described elsewhere herein. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 87%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 20, 15, 10, 9, 8, 7, 6, 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

Measurement of Biomarkers

The presence, absence and/or quantity of one or more biomarkers disclosed herein can be indicated as a value. The value can be one or more numerical values resulting from the evaluation of a sample, and can be derived, e.g., by measuring level(s) of the biomarker(s) in a sample by an assay performed in a laboratory, or from dataset obtained from a provider such as a laboratory, or from a dataset stored on a server. Biomarker levels can be measured using any of several techniques known in the art. The present disclosure encompass such techniques, and further include all subject fasting and/or temporal-based sampling procedures for measuring biomarkers.

The actual measurement of levels of the biomarkers can be determined at the protein or nucleic acid level using any method known in the art. “Protein” detection comprises detection of full-length proteins, mature proteins, pre-proteins, polypeptides, isoforms, mutations, variants, post-translationally modified proteins and variants thereof, and can be detected in any suitable manner. Levels of biomarkers can be determined at the protein level, e.g., by measuring the serum levels of peptides encoded by the gene products described herein, or by measuring the enzymatic activities of these protein biomarkers. Such methods are well-known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the biomarker genes according to the activity of each protein analyzed. For biomarker proteins, polypeptides, isoforms, mutations, and variants thereof known to have enzymatic activity, the activities can be determined in vitro using enzyme assays known in the art. Such assays include, without limitation, protease assays, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

Using sequence information provided by the public database entries for the biomarker, expression of the biomarker can be detected and measured using techniques well-known to those of skill in the art. For example, nucleic acid sequences in the sequence databases that correspond to nucleic acids of biomarkers can be used to construct primers and probes for detecting and/or measuring biomarker nucleic acids. These probes can be used in, e.g., Northern or Southern blot hybridization analyses, ribonuclease protection assays, and/or methods that quantitatively amplify specific nucleic acid sequences. As another example, sequences from sequence databases can be used to construct primers for specifically amplifying biomarker sequences in, e.g., amplification-based detection and quantitation methods such as reverse-transcription based polymerase chain reaction (RT-PCR) and PCR. When alterations in gene expression are associated with gene amplification, nucleotide deletions, polymorphisms, post-translational modifications and/or mutations, sequence comparisons in test and reference populations can be made by comparing relative amounts of the examined DNA sequences in the test and reference populations.

As an example, Northern hybridization analysis using probes which specifically recognize one or more of the disclosed sequences can be used to determine gene expression. Alternatively, expression can be measured using RT-PCR; e.g., polynucleotide primers specific for the differentially expressed biomarker mRNA sequences reverse-transcribe the mRNA into DNA, which is then amplified in PCR and can be visualized and quantified. Biomarker RNA can also be quantified using, for example, other target amplification methods, such as TMA, SDA, and NASBA, or signal amplification methods (e.g., bDNA), and the like. Ribonuclease protection assays can also be used, using probes that specifically recognize one or more biomarker mRNA sequences, to determine gene expression.

Alternatively, biomarker protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. See WO 04/056456 and WO 04/088309, each of which is hereby incorporated by reference in its entirety. In this regard, other biomarker analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other biomarker metabolites can be similarly detected using reagents that are specifically designed or tailored to detect such metabolites.

In some embodiments, a biomarker is detected by contacting a subject sample with reagents, generating complexes of reagent and analyte, and detecting the complexes. Examples of “reagents” include but are not limited to nucleic acid primers, antibodies, and antigen binding fragments.

In some embodiments, an antibody binding assay is used to detect a biomarker; e.g., a sample from the subject is contacted with an antibody reagent that binds the biomarker analyte, a reaction product (or complex) comprising the antibody reagent and analyte is generated, and the presence (or absence) or amount of the complex is determined. The antibody reagent useful in detecting biomarker analytes can be monoclonal, polyclonal, chimeric, recombinant, or a fragment of the foregoing, as discussed in detail above, and the step of detecting the reaction product can be carried out with any suitable immunoassay. The sample from the subject is typically a biological fluid as described above, and can be the same sample of biological fluid as is used to conduct the method described herein.

Immunoassays carried out in accordance with the present disclosure can be homogeneous assays or heterogeneous assays. Immunoassays carried out in accordance with the disclosure can be multiplexed. In a homogeneous assay, the immunological reaction can involve the specific antibody (e.g., anti-biomarker protein antibody), a labeled analyte, and the sample of interest. The label produces a signal, and the signal arising from the label becomes modified, directly or indirectly, upon binding of the labeled analyte to the antibody. Both the immunological reaction of binding, and detection of the extent of binding, can be carried out in a homogeneous solution. Immunochemical labels which can be employed include but are not limited to free radicals, radioisotopes, fluorescent dyes, enzymes, bacteriophages, and coenzymes. Immunoassays include competition assays.

In a heterogeneous assay approach, the reagents can be the sample of interest, an antibody, and a reagent for producing a detectable signal. Samples as described above can be used. The antibody can be immobilized on a support, such as a bead (such as protein A and protein G agarose beads), plate or slide, and contacted with the sample suspected of containing the biomarker in liquid phase. The support is separated from the liquid phase, and either the support phase or the liquid phase is examined using methods known in the art for detecting signal. The signal is related to the presence of the analyte in the sample. Methods for producing a detectable signal include but are not limited to the use of radioactive labels, fluorescent labels, or enzyme labels. For example, if the antigen to be detected contains a second binding site, an antibody which binds to that site can be conjugated to a detectable (signal-generating) group and added to the liquid phase reaction solution before the separation step. The presence of the detectable group on the solid support indicates the presence of the biomarker in the test sample. Examples of suitable immunoassays include but are not limited to oligonucleotides, immunoblotting, immunoprecipitation, immunofluorescence methods, chemiluminescence methods, electrochemiluminescence (ECL), and/or enzyme-linked immunoassays (ELISA).

Those skilled in the art will be familiar with numerous specific immunoassay formats and variations thereof which can be useful for carrying out the method disclosed herein. See, e.g., E. Maggio, Enzyme-Immunoassay (1980), CRC Press, Inc., Boca Raton, Fla. See also U.S. Pat. No. 4,727,022 to C. Skold et al., titled “Novel Methods for Modulating Ligand-Receptor Interactions and their Application”; U.S. Pat. No. 4,659,678 to G C Forrest et al., titled “Immunoassay of Antigens”; U.S. Pat. No. 4,376,110 to GS David et al., titled “Immunometric Assays Using Monoclonal Antibodies”; U.S. Pat. No. 4,275,149 to D. Litman et al., titled “Macromolecular Environment Control in Specific Receptor Assays”; U.S. Pat. No. 4,233,402 to E. Maggio et al., titled “Reagents and Method Employing Channeling”; and, U.S. Pat. No. 4,230,797 to R. Boguslaski et al., titled “Heterogenous Specific Binding Assay Employing a Coenzyme as Label.”

Antibodies can be conjugated to a solid support suitable for an assay (e.g., beads such as protein A or protein G agarose, microspheres, plates, slides or wells formed from materials such as latex or polystyrene) in accordance with known techniques, such as passive binding. Antibodies can likewise be conjugated to detectable labels or groups such as radiolabels (e.g., ³⁵S, ¹²⁵I, ¹³¹I).enzyme labels (e.g., horseradish peroxidase, alkaline phosphatase), and fluorescent labels (e.g., fluorescein, Alexa, green fluorescent protein, rhodamine) in accordance with known techniques.

Antibodies may also be useful for detecting post-translational modifications of biomarkers. Examples of post-translational modifications include, but are not limited to tyrosine phosphorylation, threonine phosphorylation, serine phosphorylation, citrullination and glycosylation (e.g., O-GlcNAc). Such antibodies specifically detect the phosphorylated amino acids in a protein or proteins of interest, and can be used in the immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF). See U. Wirth et al., Proteomics 2002, 2(10):1445-1451.

Accordingly, in some embodiments, the disclosure provides a system comprising a solid support and one or a plurality of probes complementary to one or a plurality of the biomarkers disclosed elsewhere herein. In some embodiments, the one or plurality of probes are immobilized or absorbed onto the solid support. In other embodiments, the disclosure provides a system comprising a solid support and one or a plurality of antigen binding fragments specifically bind to one or a plurality of biomarkers disclosed elsewhere herein. In some embodiments, the one or plurality of antigen binding fragments are immobilized or absorbed onto the solid support. In some embodiments, the solid support is bead, such as protein A and protein G agarose beads. In some embodiments, the solid support is plate. In some embodiments, the solid support is slide. In some embodiments, the probes are nucleic acids that are from about 5 to about 200 nucleotides in length that are complementary to any nucleotide sequence encoding a biomarker disclosed herein, such nucleotide sequence encoding a biomarker is any terminal or nested and contiguous sequence that is from about 5 to about 200 nucleotides in length and having at least about 85%, 90%, 95% 96%, 97%, 98%, 99%6 or 100% to a terminal or nested contiguous sequence of any biomarker sequence.

Rating Disease Activity (RAScore)

In some embodiments, the RAScore, derived as described herein, can be used to rate RA disease activity; e.g., as high, medium or low. The score can be varied based on a set of values chosen by the practitioner. For example, a score can be set such that a value is given a range from 0-100, and a difference between two scores would be a value of at least one point. The practitioner can then assign disease activity based on the values. For example, in some embodiments a score of 1 to 29 represents a low level of disease activity, a score of 30 to 44 represents a moderate level of disease activity, and a score of 45 to 100 represents a high level of disease activity. The disease activity score can change based on the range of the score. For example, a score of 1 to 58 can represent a low level of disease activity when a range of 0-200 is utilized. Differences can be determined based on the range of score possibilities. For example, if using a score range of 0-100, a small difference in scores can be a difference of about 1, 2, 3, 4, 5, 6, 7, 8, 9, or 10 points; a moderate difference in scores can be a difference of about 4, 5, 6, 7, 8, 9, 10, 11, 12, 13, 14, 15, 16, 17, 18, 19, 20, 21, 22, 23, 24, 25, 26, 27, 28, 29, or 30 points; and large differences can be a change in about 14, 15, 16, 17, 18, 19, 20, 21, 22, 23, 24, 25, 26, 27, 28, 29, 30, 35, 40, 45, or 50 points. Thus, by way of example, a practitioner can define a small difference in scores as about ≤6 points, a moderate difference in scores as about 7-20 points, and a large difference in scores as about >20 points. The difference can be expressed by any unit, for example, percentage points. For example, a practitioner can define a small difference as about ≤6 percentage points, moderate difference as about 7-20 percentage points, and a large difference as about >20 percentage points.

In some embodiments, arthritis disease activity can be so rated. In some embodiments, RA disease activity can be so rated. In other embodiments, osteoarthritis disease activity can be so rated. Because the RAScore correlates well with traditional clinical assessments of inflammatory disease activity, e.g. in RA, in other embodiments of the disclosure, disease progression in a subject or population can be tracked via the use and application of the RAScore.

The RAScore can be used for several purposes. On a subject-specific basis, it provides a context for understanding the relative level of disease activity. The RAScore rating of disease activity can be used, e.g., to guide the clinician in determining treatment, in setting a treatment course, and/or to inform the clinician that the subject is in remission. Moreover, it provides a means to more accurately assess and document the qualitative level of disease activity in a subject. It is also useful from the perspective of assessing clinical differences among populations of subjects within a practice. For example, this tool can be used to assess the relative efficacy of different treatment modalities. Moreover, it is also useful from the perspective of assessing clinical differences among different practices. This would allow physicians to determine what global level of disease control is achieved by their colleagues, and/or for healthcare management groups to compare their results among different practices for both cost and comparative effectiveness. Because the RAScore demonstrates strong association with established disease activity assessments, the RAScore can provide a quantitative measure for monitoring the extent of subject disease activity, and response to treatment.

Calculation of Scores

In some embodiments, arthritis or RA disease activity in a subject is measured by: determining the levels of two or more of the disclosed biomarkers in a sample of a subject known to have or suspected of having arthritis or RA, at least one of the biomarkers is up-regulated and at least one of the biomarkers is down-regulated in the subject, applying an interpretation function to transform the biomarker levels into a single RAScore, which provides a quantitative measure of arthritis or RA disease activity in the subject, correlating well with traditional clinical assessments of arthritis or RA disease activity, as is demonstrated in the Examples below. In some embodiments, the disease activity so measured relates to an autoimmune disease. In some embodiments, the disease activity so measured relates to RA.

In some embodiments, the interpretation function to transform the biomarker levels into a single RAScore is accomplished by: i) calculating a geometric mean expression of biomarkers that are up-regulated in RA patients, ii) calculating a geometric mean expression of biomarkers that are down-regulated in RA patients, and iii) calculating the RAScore by subtracting the geometric mean expression of the down-regulated biomarkers from the geometric mean expression of the up-regulated biomarkers. The biomarkers that are up-regulated in RA patients can include: TNFAIP6, S100A8, DRAM1, TNFSF10, LY96, QPCT, KYNU, ENTPD1, CLIC1 and ATP6V0E1. The biomarkers that are down-regulated in RA patients can include NCL, CIRBP and HSP90ABJ. In some embodiments, the RAScore in a subject is measured by determining the expression levels of TNFAIP6, S100A8, DRAM1, TNFSF10, LY96, QPCT, KYNU, ENTPD1, CLIC1, ATP6V0E1, NCL, CIRBP and HSP90AB1. Each of the biomarkers TNFAIP6, S100A8, DRAM1, TNFSF10, LY96, QPCT, KYNU, ENTPD1, CLIC1, ATP6V0E1, NCL, CIRBP and HSP90AB1 has the meaning as defined elsewhere herein.

Methods of Use

The disclosure further provides methods of diagnosing a subject with arthritis by detecting the presence, absence and/or quantity of one or a plurality of the biomarkers disclosed herein. In some embodiments, the disclosed method of diagnosis comprising detecting the presence, absence and/or quantity of one or a plurality of TNFAIP6, S100A8, DRAM1, TNFSF10, LY96, QPCT, KYNU, ENTPD1, CLIC1, ATP6V0E1, NCL, CIRBP and HSP90AB1 RNA transcripts in a sample from a subject. In some embodiments, the disclosed method of diagnosis comprising detecting the presence, absence and/or quantity of one or a plurality of TNFAIP6, S100A8, DRAM1, TNFSF10, LY96, QPCT, KYNU, ENTPD1, CLIC1, ATP6V0E1, NCL, CIRBP and HSP90AB1 protein in a sample from a subject. Each of the biomarkers TNFAIP6, S100A8, DRAM1, TNFSF10, LY96, QPCT, KYNU, ENTPD1, CLIC1, ATP6V0E1, NCL, CIRBP and HSP90AB1 has the meaning as defined elsewhere herein. Any methods known to one skilled in the art for detecting the presence, absence and/or quantity of one or a plurality of the disclosed biomarkers in a sample, either on the RNA level or the protein level, can be used. Exemplary methods for detection are described elsewhere herein.

In some embodiments, the disclosed method further comprises obtaining a sample from the subject. Any sample may be used. In some embodiments, the sample is a blood sample. In some embodiments, the sample is synovium.

In some embodiments, the disclosed method further comprises calculating a RAScore as described herein elsewhere. In some embodiments, the RAScore is calculated by subtracting the geometric mean expression of up-regulated biomarkers chosen from TNFAIP6, S100A8, DRAM1, TNFSF10, LY96, QPCT, KYNU, ENTPD1, CLIC1 and ATP6V0E1 from the geometric mean expression of down-regulated biomarkers chosen from NCL, CIRBP and HSP90AB1. In some embodiments, the disclosed method further comprises a step of diagnosing the subject as having arthritis if the presence, absence and/or quantity of one or a plurality of the biomarkers chosen TNFAIP6, S100A8, DRAM1, TNFSF10, LY96 QPCT, KYNU, ENTPD1, CLIC1, ATP6V0E1, NCL, CIRBP and HSP90AB1 are at a biologically significant level or levels. In some embodiments, the disclosed method further comprises a step of diagnosing the subject as having or not having RA if the presence, absence and/or quantity of one or a plurality of the biomarkers chosen from TNFAIP6, S100A8, DRAM1, TNFSF10, LY96, QPCT, KYNU, ENTPD1, CLIC1, ATP6V0E1, NCL, CIRBP and HSP90AB1 are at a biologically significant level or levels based at least on the RAScore. Each of the biomarkers TNFAIP6, S100A8, DRAM1, TNFSF10, LY96, QPCT KYNU, ENTPD1, CLIC1, ATP6V0E1, NCL, CIRBP and HSP90AB1 has the meaning as defined elsewhere herein.

The disclosure further provides methods of recommending therapeutic regimens following the diagnosis of arthritis or RA based on the determination of differences in expression of the biomarkers disclosed herein. In some embodiments, the methods of the disclosure relate to a method of distinguishing diagnoses between osteoarthritis and RA, the methods comprising any one or combination of steps disclosed herein.

In some embodiments therefore, the disclosure provides a method of treating a subject with arthritis comprising detecting the presence, absence and/or quantity of one or a plurality of the biomarkers disclosed herein as described above, and treating the subject with an arthritis treatment if the presence, absence or quantity of the one or plurality of the disclosed biomarkers is at a biologically relevant amount. In some embodiments, the biologically relevant amount is at least partially based on the calculated RAScore as described above.

Any therapies known in the art, either conventional or biologic, for arthritis or RA treatment can be used. Examples of therapies, such as disease modifying anti-rheumatic drugs (DMARD) that are generally considered conventional include, but are not limited to, MTX, azathioprine (AZA), bucillamine (BUC), chloroquine (CQ), ciclosporin (CSA, or cyclosporine, or cyclosporin), doxycycline (DOXY), hydroxychloroquine (HCQ), intramuscular gold (IM gold), leflunomide (LEF), levofloxacin (LEV), and sulfasalazine (SSZ). Conventional therapies can also include nonsteroidal anti-inflammatory drugs (NDAIDs), such as aspirin, ibuprofen, oxaprozin, prioxicam, indomethacin, etodolac, meclofenamate, meloxicam, naproxen, ketoprofen, nabumetorne, tolmetin sodium, and diclofenac. Examples of other conventional therapies include, but are not limited to, folinic acid, D-pencillamine, gold auranofin, gold aurothioglucose, gold thiomalate, cyclophosphamide, and chlorambucil. Examples of biologic drugs can include but are not limited to biological agents that target the tumor necrosis factor (TNF)-alpha molecules and the TNF inhibitors, such as infliximab, adalimumab, etanercept and golimumab. Other classes of biologic drugs include IL1 inhibitors such as anakinra, T-cell modulators such as abatacept, B-cell modulators such as rituximab, and IL6 inhibitors such as tocilizumab.

To identify additional therapeutics or drugs that are appropriate for a specific subject, a test sample from the subject can also be exposed to a therapeutic agent or a drug, and the level of one or more biomarkers can be determined. The level of one or more biomarkers can be compared to sample derived from the subject before and after treatment or exposure to a therapeutic agent or a drug, or can be compared to samples derived from one or more subjects who have shown improvements in arthritis or RA disease state or activity (e.g., clinical parameters or traditional laboratory risk factors) as a result of such treatment or exposure.

Identifying the state of arthritis or RA disease in a subject allows for a prognosis of the disease, and thus for the informed selection of, initiation of, adjustment of or increasing or decreasing various therapeutic regimens in order to delay, reduce or prevent that subject's progression to a more advanced disease state. In some embodiments, subjects can be identified as having a particular level of arthritis or RA disease activity and/or as being at a particular state of disease, based on the presence, absence and/or quantity of one or a plurality of the biomarkers disclosed here, and/or based on the determination of their RAScores, and so can be selected to begin or accelerate treatment to prevent or delay the further progression of arthritis or RA disease. In other embodiments, subjects that are identified via the presence, absence and/or quantity of one or a plurality of the disclosed biomarkers and/or their RAScores as having a particular level of arthritis or RA disease activity, and/or as being at a particular state of arthritis or RA disease, can be selected to have their treatment decreased or discontinued, where improvement or remission in the subject is seen.

Measuring RAScores derived from expression levels of the biomarkers disclosed herein over a period time can also provide a physician with a dynamic picture of a subject's biological state. These embodiments thus will provide subject-specific biological information, which will be informative for therapy decision and will facilitate therapy response monitoring, and should result in more rapid and more optimized treatment, better control of disease activity, and an increase in the proportion of subjects achieving remission.

In some embodiments, the levels of one or more disclosed biomarkers or the levels of a specific panel of disclosed biomarkers in a sample are compared to a control or reference standard (“control,” “reference standard” or “reference level”) in order to direct treatment decisions. Expression levels of the one or more biomarkers can be combined into a RAScore as calculated according to the disclosure provided elsewhere herein, which can represent disease activity. The control or reference standard used for any embodiment disclosed herein may comprise average, mean, or median levels of the one or more biomarkers or the levels of the specific panel of biomarkers in a control population. The control population can be a population of heathy subjects known to not have arthritis or RA. In such embodiments, a higher RAScore is indicative that the subject has arthritis or RA. The control population can also be a population of subjects known to have a certain subtype of arthritis. In such embodiments, a higher or lower RAScore is indicative that the subject has a subtype of arthritis that is different from the subtype of arthritis the control population has.

In some embodiments therefore, the disclosure provides a method of identifying prognosis of arthritis in a subject in need thereof, the method comprising detecting the presence, absence and/or quantity of one or a plurality of the biomarkers disclosed herein as described above. In some embodiments, the method of identifying prognosis of arthritis in the subject further comprises calculating a RAScore as described above. In some embodiments, the method further comprises comparing the calculated RAScore with a control RAScore calculated from a control dataset obtained from healthy subjects, wherein a higher calculated RAScore is indicative that the subject has arthritis.

In other embodiments, the disclosure provides a method of classifying a subject with a subtype of arthritis, the method comprising detecting the presence, absence and/or quantity of one or a plurality of the biomarkers disclosed herein and calculating a RAScore as described above. In some embodiments, the method further comprises the calculated RAScore with a control RAScore calculated from a control dataset obtained from subjects known to have osteoarthritis, wherein a a higher calculated RAScore is indicative that the subject has RA.

The control or reference standard may also be an earlier time point for the same subject. For example, a control or reference standard may include a first time point, and the levels of the one or more biomarkers can be examined again at second, third, fourth, fifth, sixth time points, etc. Any time point earlier than any particular time point can be considered a control or reference standard. The control or reference standard may additionally comprise cutoff values or any other statistical attribute of the control population, or earlier time points of the same subject, such as a standard deviation from the mean levels of the one or more biomarkers or the levels of the specific panel of biomarkers. In some embodiments, the control population may comprise healthy individuals or the same subject prior to the administration of any therapy.

In some embodiments, a RAScore may be obtained from the reference time point, and a different RAScore may be obtained from a later time point. A first time point can be when an initial therapeutic regimen is begun. A first time point can also be when a first immunoassay is performed. A time point can be hours, days, months, years, etc. In some embodiments, a time point is one month. In some embodiments, a time point is two months. In some embodiments, a time point is three months. In some embodiments, a time point is four months. In some embodiments, a time point is five months. In some embodiments, a time point is six months. In some embodiments, a time point is seven months. In some embodiments, a time point is eight months. In some embodiments, a time point is nine months. In some embodiments, a time point is ten months. In some embodiments, a time point is eleven months. In some embodiments, a time point is twelve months. In some embodiments, a time point is two years. In some embodiments, a time point is three years. In some embodiments, a time point is four years. In some embodiments, a time point is five years. In some embodiments, a time point is ten years.

A difference in the RAScore can be interpreted as an increase or decrease in disease activity. For example, a second RAScore having a lower score than the reference RAScore, or first RAScore, means that the subject's disease activity has been lowered (improved) between the first and second time periods. Alternatively, in the circumstances where a second RAScore having a higher score than the reference RAScore, or first RAScore, means that the subject's disease activity has been increased (worsened) between the first and second time periods.

In some embodiments therefore, the disclosure provides a method of monitoring the effectiveness of a treatment in a subject having arthritis, the method comprising detecting the presence, absence and/or quantity of one or a plurality of the biomarkers disclosed herein and calculating a RAScore as described above, wherein a lower post-treatment RAScore as compared to the pre-treatment RAScore is indicative that the treatment is effective.

In some embodiments, methods of the disclosure include methods of processing or analyzing a sample, the method comprising: a) obtaining a sample; (b) exposing the sample to one or more systems disclosed herein; (c) detecting the expression of biomarkers in the sample; (d) creating an expression profile of a sample; and analyzing the expression profile. In some embodiments, the system comprises at least one processor and a memory and the step of analyzing the expression profile comprises the following steps, each of which may be optionally performed by at least one processor: (i) creating a test data set and a training data set from an input set of data, wherein the input set of data comprises gene expression profiles of subjects having the disorder or disease and control subjects;
(ii) identifying one or a plurality of significant expression profiles correlated with the disorder or disease in the training data set using a statistical test;
(iii) evaluating expression performance of each of the significant expression profiles by applying one or a plurality of machine learning methods to create a performance algorithm;
(iv) testing the performance algorithm on the test data set;
(v) selecting a high performing expression profile corresponding to at least one biomarker based upon a first threshold of the performance algorithm;
(vi) testing the high performing expression profile selected in step (v) with a dataset, said dataset being independent from the input set of data;
(vii) and
(viii) selecting a biomarker associated with the disorder or disease based on a second threshold of the performance algorithm.

The disclosure also relates to a computer-implemented method of selecting biomarkers associated with a disorder or disease, in a system configured to host a webpage and/or compile datasets; wherein the system comprises at least one processor and a memory, the method comprising:
(i) creating, by the at least one processor, a test data set and a training data set from an input set of data, wherein the input set of data comprises gene expression profiles of subjects having the disorder or disease and control subjects;
(ii) identifying one or a plurality of significant expression profiles correlated with the disorder or disease in the training data set using a statistical test;
(iii) evaluating expression performance of each of the significant expression profiles by applying one or a plurality of machine learning methods to create a performance algorithm;
(iv) testing the performance algorithm on the test data set;
(v) selecting a high performing expression profile corresponding to at least one biomarker based upon a first threshold of the performance algorithm;
(vi) testing the high performing expression profile selected in step (v) with a dataset, said dataset being independent from the input set of data; and
(vii) selecting a biomarker associated with the disorder or disease based on a second threshold of the performance algorithm.
   In some embodiments, one or a plurality of each step is performed by the at least one processor. In any of the aforementioned methods, the methods comprise a step of diagnosing a subject with arthritis by comparing the expression profile from the sample of a subject with the expression profile of a control subject.

The disclosure also relates to a computer-implemented method of selecting biomarkers associated with a disorder or disease, in a system configured to compile datasets; wherein the system comprises at least one processor and a memory, the method comprising:
(i) creating, by the at least one processor, a test data set and a training data set from an input set of data, wherein the input set of data comprises gene expression profiles of subjects having the disorder or disease and control subjects;
(ii) identifying one or a plurality of significant expression profiles correlated with the disorder or disease in the training data set using a statistical test;
(iii) evaluating expression performance of each of the significant expression profiles by applying one or a plurality of machine learning methods to create a performance algorithm;
(iv) testing the performance algorithm on the test data set;
(v) selecting a high performing expression profile corresponding to at least one biomarker based upon a first threshold of the performance algorithm;
(vi) testing the high performing expression profile selected in step (v) with a dataset, said dataset being independent from the input set of data; and
(vii) selecting a biomarker associated with the disorder or disease based on a second threshold of the performance algorithm.

Systems

The above-described methods can be implemented in any of numerous ways. For example, embodiments of the disclosure may be implemented using a computer program product (i.e. software), hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device. Embodiments including methods of diagnosing or processing a sample may be used with a solid support in combination with a computer program product that is capable of analyzing the results of hybridization of nucleotide sequences encoding the disclosed biomarkers or association of antibodies or antibody fragments on a solid support that bind the biomarkers disclosed herein.

Certain embodiments of the invention can make use of solid supports included of an inert substrate or matrix (e.g., glass slides, polymer beads etc.) which has been functionalized, for example, by application of a layer or coating of an intermediate material including reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g., polynucleotides) can be directly covalently attached to the intermediate material (e.g., the hydrogel) but the intermediate material can itself be non-covalently attached to the substrate or matrix (e.g., the glass substrate). The term “covalent attachment to a solid support” is to be interpreted accordingly as encompassing this type of arrangement.

The terms “solid surface,” “solid support” and other grammatical equivalents herein refer to any material that is appropriate for or can be modified to be appropriate for the attachment of the target nucleotide sequences encoding biomarkers or biomarkers themselves, or variants or functional fragments thereof. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flow cell apparatus. Exemplary flow cells are set forth in further detail below.

In some embodiments, the solid support includes a patterned surface suitable for immobilization of capture primers in an ordered pattern. A “patterned surface” refers to an arrangement of different regions in or on an exposed layer of a solid support. For example, one or more of the regions can be features where one or more capture primers are present. The features can be separated by interstitial regions where capture primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the capture primers are randomly distributed upon the solid support. In some embodiments, the capture primers are distributed on a patterned surface. Exemplary patterned surfaces that can be used in the methods and compositions set forth herein are described in U.S. Ser. No. 13/661,524 or US Pat. App. Publ. No. 2012/0316086 A1, each of which is incorporated herein by reference.

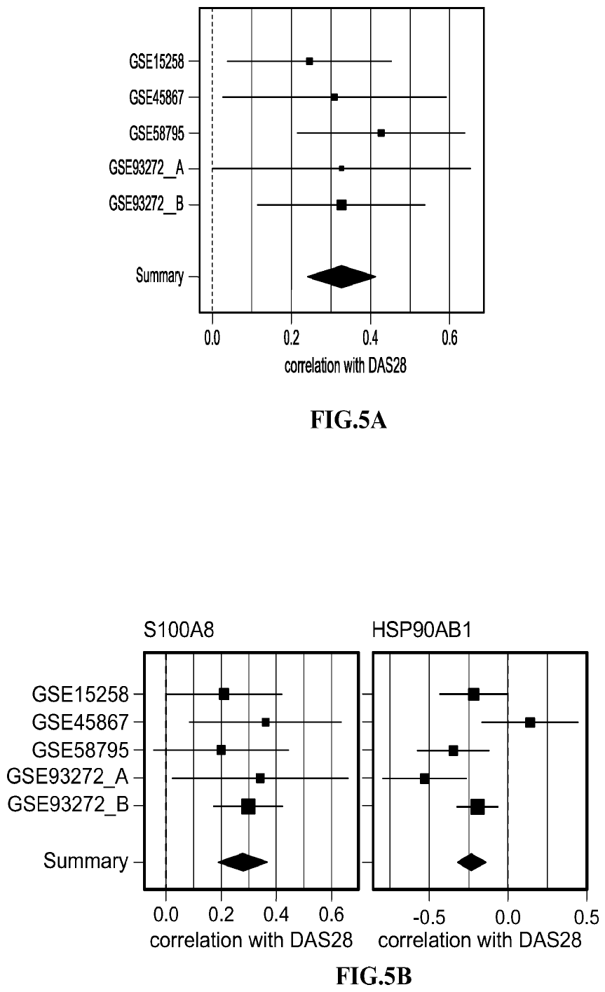

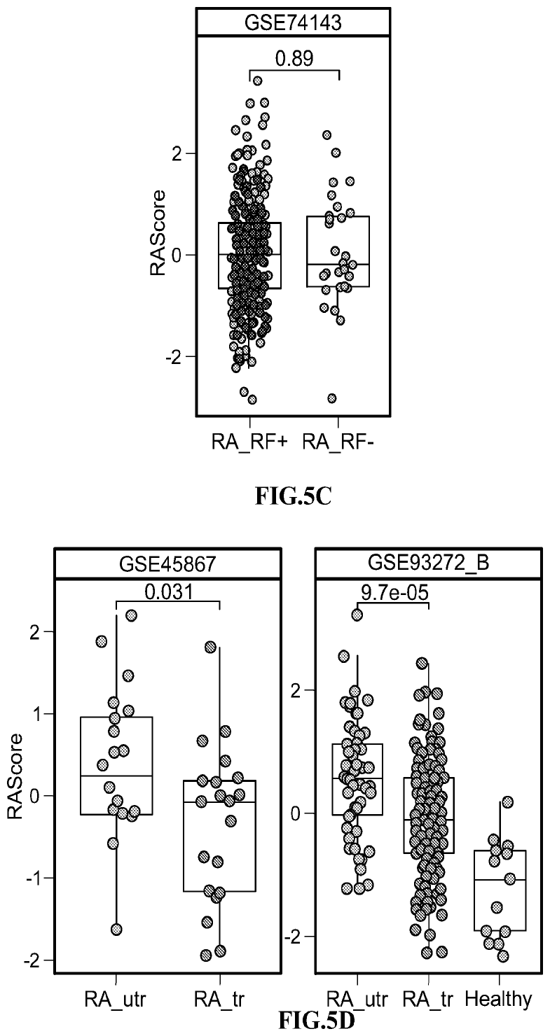

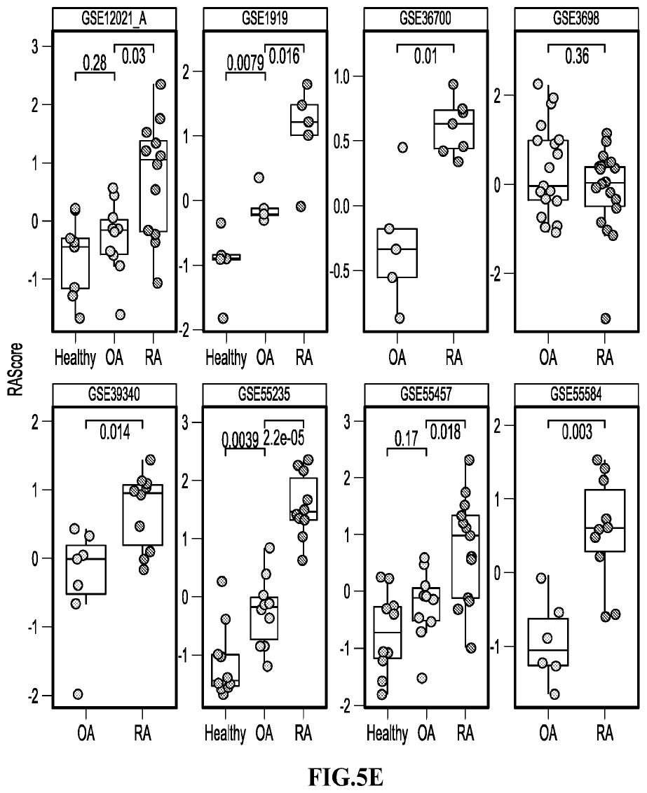

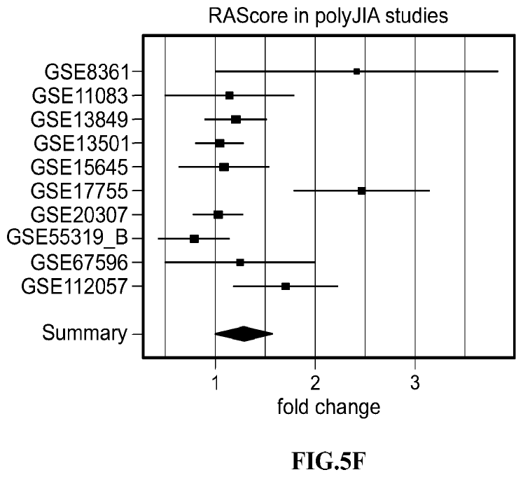

In some embodiments, the system comprises a solid support comprising one or a plurality of probes, antibodies, antibody fragments, and/or complementary nucleotide sequences specific for one or a plurality of the biomarkers disclosed herein, wherein the nucleotide sequences specific for one or a plurality of biomarkers disclosed herein are complementary to at least one nucleotide sequence encoding a biomarker with a region of from about 5 to about 100 or more nucleotides that are complementary to the nucleotide sequence that encodes the biomarkers disclosed herein; and wherein the antibody or antibody fragments are capable of associating with biomarkers that are amino acid sequences disclosed herein. In some embodiments, the probes for the biomarkers are positioned in spate discrete locations on the same reaction surface of the solid support. Samples can be run over the solid support to quantify and, in some cases, amplify semi-quantitatively or quantitatively the nucleotide sequences that encode the one or plurality of biomarkers. A growing number of next generation sequencing applications require the target-specific capture of target-specific polynucleotides (e.g. those that encode the biomarkers disclosed herein) and therefore the immobilization of target-specific capture primers besides universal capture primers on the same surface. In another example, sequence tagmentation applications require the presence of universal capture primers, and also the presence of application-specific capture primers that have transposon ends (TE) and hybridize with transposon end oligonucleotides. In some embodiments, the target-specific capture primers next to universal capture primers, wherein the universal capture primers are immobilized directly to the solid support and wherein the target-specific primers are next to or comprise a region complementary to the universal capture primers and a second region complementary to the nucleotide sequence encoding the one or plurality of biomarkers. In some embodiments, the solid support uses direct target capture. Direct target capture can be achieved by immobilizing target-specific capture primers (complementary to a portion of the nucleotide sequence encoding a disclosed biomarker) on a surface that specifically hybridize with a target polynucleotide, e.g., a polynucleotide encoding one or a plurality of biomarkers disclosed herein. In applications where many target polynucleotides need to be captured on the same flow cell (e.g., a plurality of polynucleotides encoding biomarkers or functional fragments or variants of biomarkers) the target-specific capture primers are necessarily many and varied. A high concentration of target-specific capture primers on a solid support would make target capture fast, efficient and robust. Speed, efficiency and robustness are especially important where the target polynucleotides are extremely rare and have a low abundance, for example in the case of target polynucleotides encoding somatic mutations of human biomarkers. In general, only specifically captured target polynucleotides can efficiently support bridge amplification. By contrast polynucleotides that are mishybridized to a mismatched capture primer can be inefficient in supporting capture primer extension. As a result, the mismatched polynucleotide can be inefficiently copied or amplified (see, e.g., FIG. 5). Therefore, in order to ensure efficient amplification, a large excess of universal capture primers would have to be combined on the solid support with only a small number of target-specific capture primers. Moreover, it would be necessary to carefully choose a density of target-specific capture primers that is adequate to capture the target polynucleotide but not so high as to impede the subsequent amplification step. IN some embodiments, the solid support comprises from about 10 to about 100 or more target capture nucleotides immobilized directly or indirectly on the solid support at discrete locations that are addressable with one or a number of probes that are quantified by wavelength absorption of fluorescence, chemiluminescence, or other colorimetric data collected by other components of the system. For instance, in some embodiments, the system comprises a solid support comprising one or a combination of probes, antibodies, antibody fragments specific for a biomarker disclosed herein or nucleotides complementary to a nucleotide sequence encoding a biomarker disclosed herein and a computer.

In some embodiments, the solid support includes an array of wells or depressions in a surface. This can be fabricated as is generally known in the art using a variety of techniques, including, but not limited to, photolithography, stamping techniques, molding techniques and microetching techniques. As will be appreciated by those in the art, the technique used will depend on the composition and shape of the array substrate. The composition and geometry of the solid support can vary with its use. In some embodiments, the solid support is a planar structure such as a slide, chip, microchip and/or array. As such, the surface of a substrate can be in the form of a planar layer. In some embodiments, the solid support includes one or more surfaces of a flowcell. The term “flowcell” as used herein refers to a chamber including a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, the solid support or its surface is non-planar, such as the inner or outer surface of a tube or vessel. In some embodiments, the solid support includes microspheres or beads. By “microspheres” or “beads” or “particles” or grammatical equivalents herein is meant small discrete particles. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports can all be used. “Microsphere Detection Guide” from Bangs Laboratories, Fishers Ind. is a helpful guide. In certain embodiments, the microspheres are magnetic microspheres or beads. The beads need not be spherical; irregular particles can be used. Alternatively or additionally, the beads can be porous. The bead sizes range from nanometers, e.g., 100 nm, to millimeters, e.g. 1 mm, with beads from about 0.2 micron to about 200 microns being preferred, and from about 0.5 to about 5 micron being particularly preferred, although in some embodiments smaller or larger beads can be used. Provided herein are methods of modifying an immobilized capture primer, including a) providing a solid support having an immobilized application-specific capture primer, the application-specific capture primer including i) a 3′ portion including an application-specific capture region, and ii) a 5′ portion including a universal capture region; b) contacting an application-specific polynucleotide with the application-specific capture primer under conditions sufficient for hybridization to produce an immobilized application-specific polynucleotide; and c) removing the application-specific capture region of an application-specific capture primer not hybridized to an application-specific polynucleotide to convert the unhybridized application-specific capture primer to a universal capture primer.

A computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may include a memory, coupled to one or more processing units (also referred to herein simply as “processors”), one or more communication interfaces, one or more display units, and one or more user input devices. In some embodiments, the memory may execute stpes for correlating the intensity of wavelength absorption at a given location on the solid support with the quantity of biomarker in the sample. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the disclosure relates to various embodiments in which one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In some embodiments, the disclosure relates to a computer program product encoded on a computer-readable storage medium comprising instructions for executing any of the disclosed method of selecting a biomarker as described above. In some embodiments, the disclosure relates to a system that comprises the disclosed computer program product, at least one processor, a program storage, such as memory, for storing program code executable on the processor, and one or more input/output devices and/or interfaces, such as data communication and/or peripheral devices and/or interfaces. In some embodiments, the user device and computer system or systems are communicably connected by a data communication network, such as a Local Area Network (LAN), the Internet, or the like, which may also be connected to a number of other client and/or server computer systems. The user device and client and/or server computer systems may further include appropriate operating system software.

In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java™, BASIC, Pascal, Fortran, Cobol, assembly language, machine code, or the like.

Many of the functional units described in this specification have been labeled as circuits, in order to more particularly emphasize their implementation independence. For example, a circuit may be implemented as a hardware circuit comprising custom very-large-scale integration (VLSI) circuits or gate arrays, off-the-shelf semiconductors such as logic chips, transistors, or other discrete components. A circuit may also be implemented in programmable hardware devices such as field programmable gate arrays, programmable array logic, programmable logic devices or the like.

In some embodiment, the circuits may also be implemented in machine-readable medium for execution by various types of processors. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

The computer readable medium (also referred to herein as machine-readable media or machine-readable content) may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. As alluded to above, examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. As also alluded to above, computer readable program code embodied on a computer readable signal medium may be transmitted using any appropriate medium, including but not limited to wireless, wireline, optical fiber cable, Radio Frequency (RF), or the like, or any suitable combination of the foregoing. In one embodiment, the computer readable medium may comprise a combination of one or more computer readable storage mediums and one or more computer readable signal mediums. For example, computer readable program code may be both propagated as an electromagnetic signal through a fiber optic cable for execution by a processor and stored on RAM storage device for execution by the processor.

Computer readable program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The computer readable program code may execute entirely on a user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an extemal computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks attached as Figures. In some embodiments, the program code execute steps to compile subject data and select biomarkers associated with a particular disorder or disease.

Functions, operations, components and/or features described herein with reference to one or more embodiments, may be combined with, or may be utilized in combination with, one or more other functions, operations, components and/or features described herein with reference to one or more other embodiments, or vice versa.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure.

In some embodiments, the disclosure relates to a system comprising a computer program product that executes step for a method to select one or a plurality of biomarkers, the method comprising method of selecting a biomarker associated with a disorder or disease, the method comprising:
a) creating a test data set and a training data set from an input set of data, wherein the input set of data comprises gene expression profiles of subjects having the disorder or disease and control subjects;
b) identifying one or a plurality of significant expression profiles correlated with the disorder or disease in the training data set using a statistical test;
c) evaluating expression performance of each of the significant expression profiles by applying one or a plurality of machine learning methods to create a performance algorithm;
d) testing the performance algorithm on the test data set;
e) selecting a high performing expression profile corresponding to at least one biomarker based upon a first threshold of the performance algorithm;
f) testing the high performing expression profile selected in step e) with a dataset, said dataset being independent from the input set of data; and
g) selecting a biomarker associated with the disorder or disease based on a second threshold of the performance algorithm. In some embodiments, the executable method is a machine-learning tool that simulates or executes the steps repeatedly over time until about 1, 2, 3, 4, 5, 6, 7, 8, 9, 10, 11, 12, 13, 14, 15, 16, 17, 18, 19, or about 20 or more biomarkers are selected as being associated with the disorder or disease state. In some cases the data are taken from a series of control subjects. In some embodiments, the data are taken from a series of experimental subject that have been diagnosed or are suspected as having a particular disease or disorder. In some embodiments, the disease is arthritis. In some embodiments, the disease is RA or osteoarthiritis.

Exemplary methods for array-based expression and genotyping analysis that can be applied to detection according to the present disclosure are described in U.S. Pat. Nos. 7,582,420; 6,890,741; 6,913,884 or 6,355,431 or US Pat. Pub. Nos. 2005/0053980 A1; 2009/0186349 A1 or US 2005/0181440 A1. A beneficial use of the methods set forth herein is that they provide for rapid and efficient detection of a plurality of nucleic acid fragments in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized nucleic acid fragments, the system including components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeg™ platform (Illumina®, Inc., San Diego, Calif.) and devices described in U.S. Ser. No. 13/273,666.

All referenced journal articles, patents, and other publications are incorporated by reference herein in their entireties.

### EXAMPLES

### Example 1. Cross-Tissue Transcriptomic Analysis Leveraging Machine Learning Approaches Identifies New Biomarkers for Rheumatoid Arthritis

In this study, we leveraged publicly available transcriptomic datasets generated from microarray and RNA sequencing (RNAseq) platforms from over 2,000 samples from whole blood and synovial tissue of patients with RA. After combining these datasets in using a well-described meta-analytic pipeline and describing the expression pathways and cell types present in RA tissues, we developed a robust machine learning and feature selection approach to identify unique and independent biomarkers which were subsequently refined and validated on test data. We then evaluated the diagnostic utility of this set of biomarkers and the correlation with disease activity measures to inform future clinical studies. The development of an objective blood test for the diagnosis and monitoring of RA can add valuable information to the physician's assessment and help inform decision-making to improve the morbidity and quality of life for patients with RA.

1. Materials and Methods

i. Discovery Data Collection and Processing

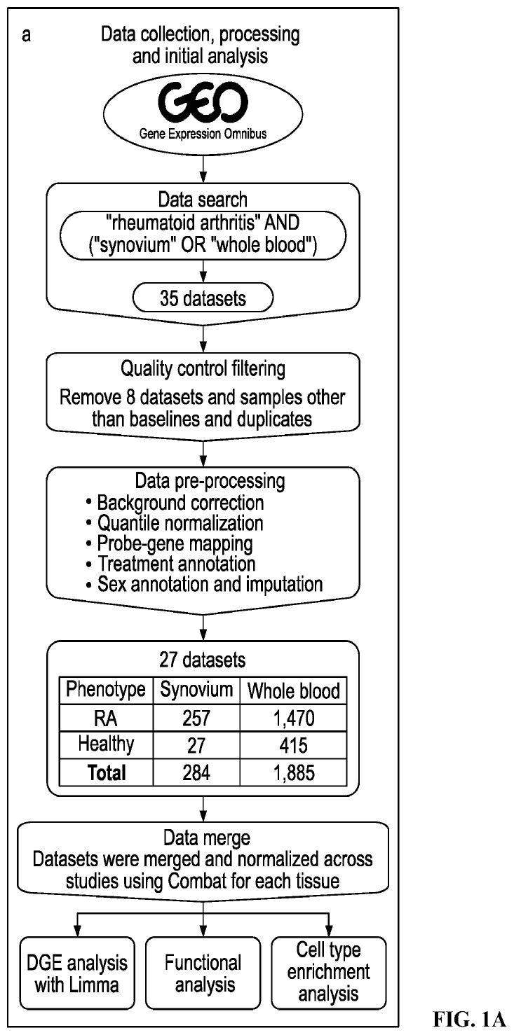

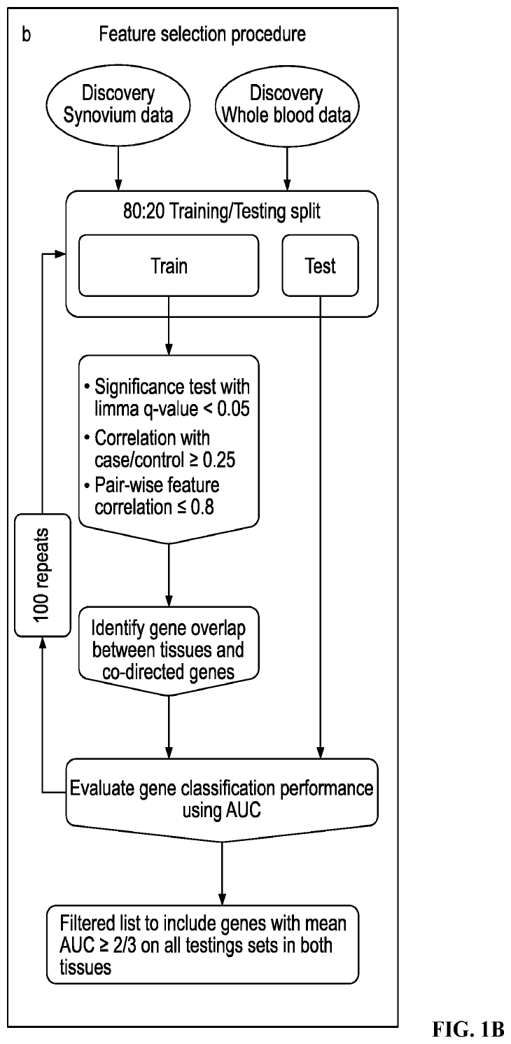

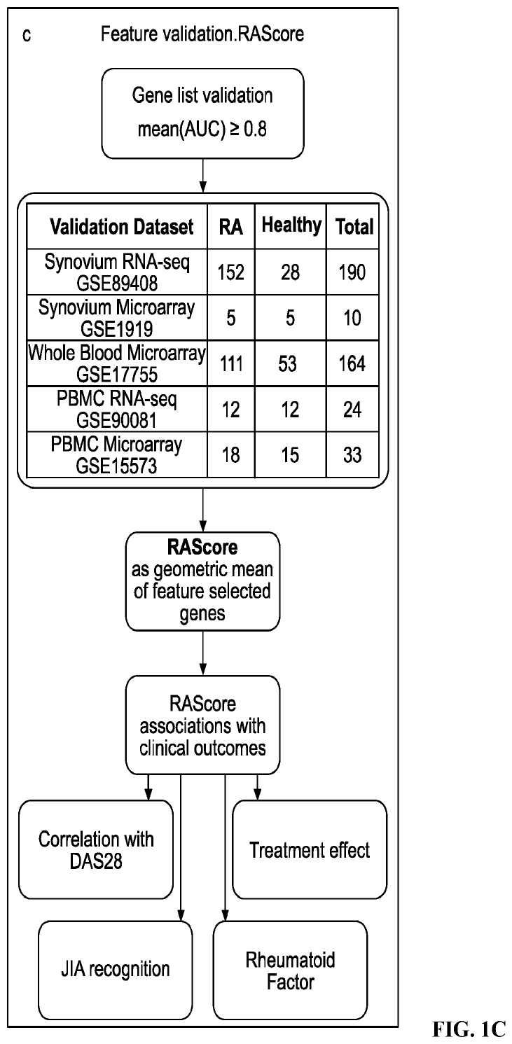

We carried out a comprehensive search for publicly available microarray data at NCBI Gene Expression Omnibus (GEO) database (http://www.ncbi.nlm.nih.gov/geo/) for whole blood and synovial tissues in Rheumatoid Arthritis and healthy controls using the keywords “rheumatoid arthritis,” “synovium,” “synovial,” “biopsy” and “whole blood,” among organisms “Homo Sapiens” and study type “Expression profiling by array” (FIG. 1A). Datasets were excluded when samples were poorly annotated or run on platforms with few numbers of probes. This search yielded 13 synovial datasets, which included 257 biopsy samples from subjects with RA and 27 from healthy controls obtained during joint or trauma surgeries (Table 1). Fourteen whole blood datasets with 1,885 samples, 1,470 RA patients and 415 healthy controls, were identified (Table 1).

TABLE 1
Overview of the Discovery and Validation Studies

| study | platform | used for | Tissue | total | Healthy | RA | OA | poly [symbol] | PMID | Country | Year |
|---|---|---|---|---|---|---|---|---|---|---|---|
| GSE12021 | GPL96 [HG-U133A] | discovery | Synovium | 31 | 9 | 12 | 10 | | 1 [symbol] 721452 | Germany | 200 [symbol] |
| | [symbol] Human Genome | | | | | | | | | | |
| | U133A Array | | | | | | | | | | |
| GSE15 [symbol] | GPL570 [HG-U133_Plus_2] | discovery | Synovium | 11 | | 11 | | | [symbol] | Belgium | 2009 |
| | [symbol] Human Genome U133 | | | | | | | | | | |
| | Plus 2.0 Array | | | | | | | | | | |
| GSE21537 | GPL7768 KTH | discovery | Synovium | 62 | | 62 | | | [symbol] | Sweden | 2010 |
| | Human [symbol] | | | | | | | | | | |
| GSE24742 | GPL570 [HG-U133_Plus_2] | discovery | Synovium | 12 | | 12 | | | 21337318 | Belgium | 2010 |
| | [symbol] Human Genome U133 | | | | | | | | | | |
| | Plus 2.0 Array | | | | | | | | | | |
| GSE36700 | GPL570 [HG-U133_Plus_2] | discovery | Synovium | 12 | | 7 | 5 | | 17489140 | Belgium | 2012 |
| | [symbol] Human Genome U133 | | | | | | | | | | |
| | Plus 2.0 Array | | | | | | | | | | |
| GSE39340 | GPL10558 [symbol] | discovery | Synovium | 17 | | 10 | 7 | | [symbol] | China | 2012 |
| GSE [symbol] | GPL570 | discovery | Synovium | 20 | | 2 [symbol] | | | [symbol] 9571 | Belgium | 2013 |
| | [HG-U133_Plus_2] [symbol] | | | | | | | | | | |
| | Human Genome U133 | | | | | | | | | | |
| | Plus 2.0 Array | | | | | | | | | | |
| GSE48780 | GPL570 [HG-U133A] | discovery | Synovium | 83 | | 83 | | | 24935 [symbol] | USA | 2013 |
| | [symbol] Human | | | | | | | | | | |
| | Genome U133A Array | | | | | | | | | | |
| GSE55235 | GPL [symbol] [HG-U133_Plus_2] | discovery | Synovium | 30 | 10 | 10 | 10 | | [symbol] 414 | Germany | 2014 |
| | [symbol] human Genome U133 | | | | | | | | | | |
| | Plus 2.0 Array | | | | | | | | | | |
| GSE55457 | GPL96 [HG-U133A] | discovery | Synovium | 22 | 1 | 1 | 10 | | 24690414 | Germany | 2014 |
| | [symbol] Human | | | | | | | | | | |
| | Genome U133A Array | | | | | | | | | | |
| GSE55584 | GPL96 [HG-U133A] | discovery | Synovium | 1 [symbol] | | 10 | 6 | | [symbol] | Germany | 2014 |
| | [symbol] Human | | | | | | | | | | |
| | Genome U133A Array | | | | | | | | | | |
| GSE57376 | GPL13158 [HT_NG- | discovery | Synovium | 3 | | 3 | | | 25333715 | USA | 2014 |
| | U133_Plus_PM] [symbol] | | | | | | | | | | |
| GSE77296 | GPL570 | discovery | Synovium | 23 | 7 | 16 | | | 26711533 | Netherlands | 2016 |
| | [HG-U133_Plus_2] [symbol] Human | | | | | | | | | | |
| | Genome U123 Plus 2.0 Array | | | | | | | | | | |
| GSE12051 | GPL2507 [symbol] | discovery | Whole | 44 | | 44 | | | 19847310 | Spain | 2008 |
| | Human-6 | | blood | | | | | | | | |
| | Expression | | | | | | | | | | |
| | BreadChip | | | | | | | | | | |
| GSE [symbol] | GPL570 | discovery | Whole | 86 | | 86 | | | 19699293 | USA | 2009 |
| | [HG-U133_Plus_2] [symbol] | | blood | | | | | | | | |
| | Human Genome U133 | | | | | | | | | | |
| | Plus 2.0 Array | | | | | | | | | | |
| GSE37107 | GPL6947 [symbol] | discovery | Whole | 14 | | 14 | | | 22540992 | Netherlands | 2012 |
| | HumanHT-12 [symbol] | | blood | | | | | | | | |
| GSE45291 | GPL13158 | discovery | Whole | 513 | 20 | 493 | | | 25405351 | USA | 2013 |
| | [HT_HG-U133_Plus_PM] [symbol] | | blood | | | | | | | | |
| GSE47727 | GPL5947 [symbol] | discovery | Whole | 122 | 122 | | | | 24013839 | USA | 2013 |
| | expression | | blood | | | | | | | | |
| | breadchip | | | | | | | | | | |
| GSE47728 | GPL10558 [symbol] | discovery | Whole | 228 | 228 | | | | 24013839 | USA | 2013 |
| | expression | | blood | | | | | | | | |
| | breadchip | | | | | | | | | | |
| GSE54629 | GPL5244 [symbol] | discovery | Whole | 69 | | 69 | | | [symbol] | France | 2014 |
| | | | blood | | | | | | | | |
| GSE58795 | GPL [symbol] | discovery | Whole | 59 | | 59 | | | 255 [symbol] | USA | 2014 |
| | | | blood | | | | | | | | |
| GSE38215 | GPL4133 [symbol] | discovery | Whole | 36 | | 36 | | | 285 [symbol] | France | 2015 |
| | Whole Human [symbol] | | blood | | | | | | | | |
| [symbol] | GPL20171 | discovery | Whole | 15 | 5 | 10 | | | [symbol] | USA | 2015 |
| | [symbol] | | blood | | | | | | | | |
| GSE741 [symbol] 3 | GPL13158 | discovery | Whole | 377 | | 377 | | | 27140173 | USA | 2015 |
| | [HT_HG-U133_Plus_PM] | | blood | | | | | | | | |
| | [symbol] HG-U133 [symbol] | | | | | | | | | | |
| GSE [symbol] | GPL6480 [symbol] | discovery | Whole | 209 | | 209 | | | 27435242 | Japan | 2016 |
| | Human Genome [symbol] | | blood | | | | | | | | |
| GSE92272 | GPL570 | discovery | Whole | 101 | 35 | 66 | | | 3001302 [symbol] | Japan | 2017 |
| | [HG-U133_Plus_2] | | blood | | | | | | | | |
| | [symbol] Human | | | | | | | | | | |
| | Genome U133 | | | | | | | | | | |
| | Plus 2.0 Array | | | | | | | | | | |
| GSE150191 | GPL13497 | discovery | Whole | 12 | 5 | 7 | | | 29584756 | Mexico | 2017 |
| | [symbol] Whole Human | | blood | | | | | | | | |
| | Genome [symbol] | | | | | | | | | | |
| GSE1619 | GPL91 | validation | Synovium | 15 | 5 | 5 | 5 | | 20858714 | Germany | 2004 |
| | [HG_U95A] | | | | | | | | | | |
| | [symbol] Human | | | | | | | | | | |
| | Genome U59A | | | | | | | | | | |
| GSE [symbol] | GPL11 [symbol] 4 [symbol] 2000 | validation | Synovium | 180 | 26 | 152 | | | 28455435 | USA | 2016 |
| GSE15573 | GPL5102 [symbol] | validation | PBMC | 33 | 1 [symbol] | 18 | | | [symbol] | France | 2009 |
| GSE17755 | GPL1291 [symbol] | validation | Whole | 164 | 53 | 111 | | 6 | 214 [symbol] | Japan | 2009 |
| | | | Blood | | | | | | | | |
| GSE [symbol] | GPL11154 [symbol] 2000 | validation | PBMC | 24 | 12 | 12 | | | 2814 [symbol] | Sweden | 2016 |
| GSE [symbol] | GPL [symbol] | misc. | Synovium | 48 | | 18 | 19 | | [symbol] | Germany | 2005 |
| | Human [symbol] | | | | | | | | | | |
| GSE8361 | GPL1291 [symbol] | misc. | PBMC | 14 | 8 | | | 6 | [symbol] | Japan | 2007 |
| GSE11083 | GPL570 | misc. | PBMC | 29 | 15 | | | 14 | 19236715 | USA | 2008 |
| | [HG-U133_Plus_2] | | | | | | | | | | |
| | [symbol] Human | | | | | | | | | | |
| | Genome U133 | | | | | | | | | | |
| | Plus 2.0 Array | | | | | | | | | | |
| GSE13840 | GPL570 | misc. | PBMC | 120 | 59 | | | [symbol] 1 | 19565504 | USA | 2008 |
| | [HG-U133_Plus_2] | | | | | | | | | | |
| | [symbol] Human | | | | | | | | | | |
| | Genome U133 | | | | | | | | | | |
| | Plus 2.0 Array | | | | | | | | | | |
| GSE [symbol] | GPL570 | misc. | PBMC | 104 | 59 | | | 45 | 19365513 | USA | 2008 |
| | [HG-U133_Plus_2] | | | | | | | | | | |
| | [symbol] Human | | | | | | | | | | |
| | Genome U133 | | | | | | | | | | |
| | Plus 2.0 Array | | | | | | | | | | |
| GSE15845 | GPL570 | misc. | PBMC | 42 | 13 | | | 29 | 19248118 | USA | 2009 |
| | [HG-U133_Plus_2] | | | | | | | | | | |
| | [symbol] Plus 2.0 Array | | | | | | | | | | |
| GSE20307 | GPL570 | misc. | PBMC | 100 | 56 | | | 44 | 20662067 | USA | 2010 |
| | [HG-U133_Plus_2] | | | | | | | | | | |
| | [symbol] Plus 2.0 Array | | | | | | | | | | |
| GSE [symbol] | GPL10558 | misc. | Whole | 45 | 19 | | | 26 | 24782192 | USA | 2014 |
| | [HG-U133_Plus_2] | | Blood | | | | | | | | |
| | [symbol] | | | | | | | | | | |
| GSE [symbol] | GPL570 [symbol] | misc. | PBMC | 20 | 15 | | | 14 | [symbol] | USA | 2015 |
| | Plus 2.0 Array | | | | | | | | | | |
| GSE112057 | GPL11154 [symbol] | misc. | Whole | 55 | 12 | | | 46 | [symbol] | USA | 2018 |
| | | | Blood | | | | | | | | |

[symbol]

indicates data missing or illegible when filed

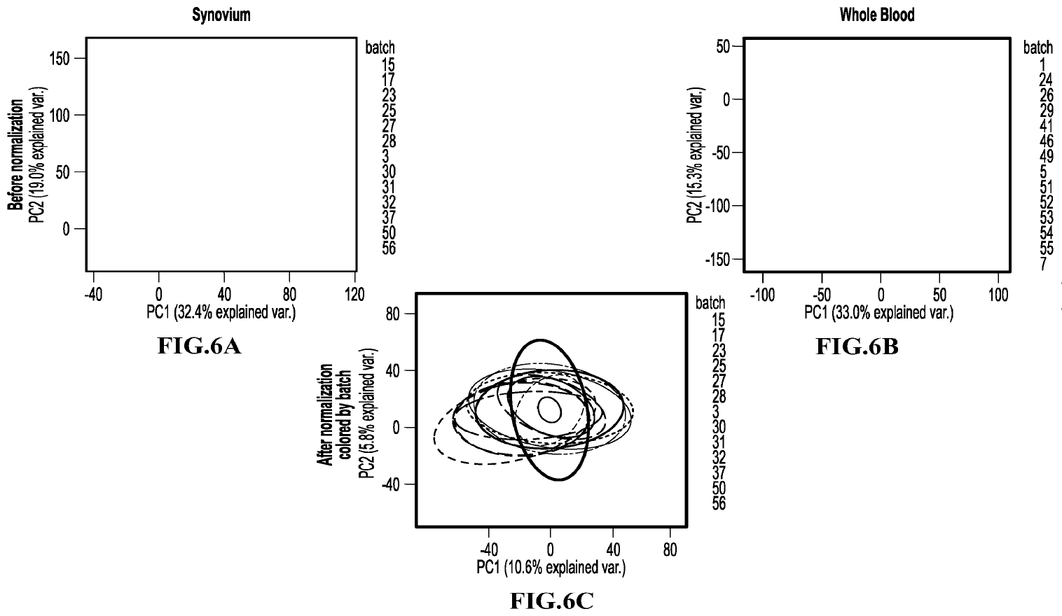

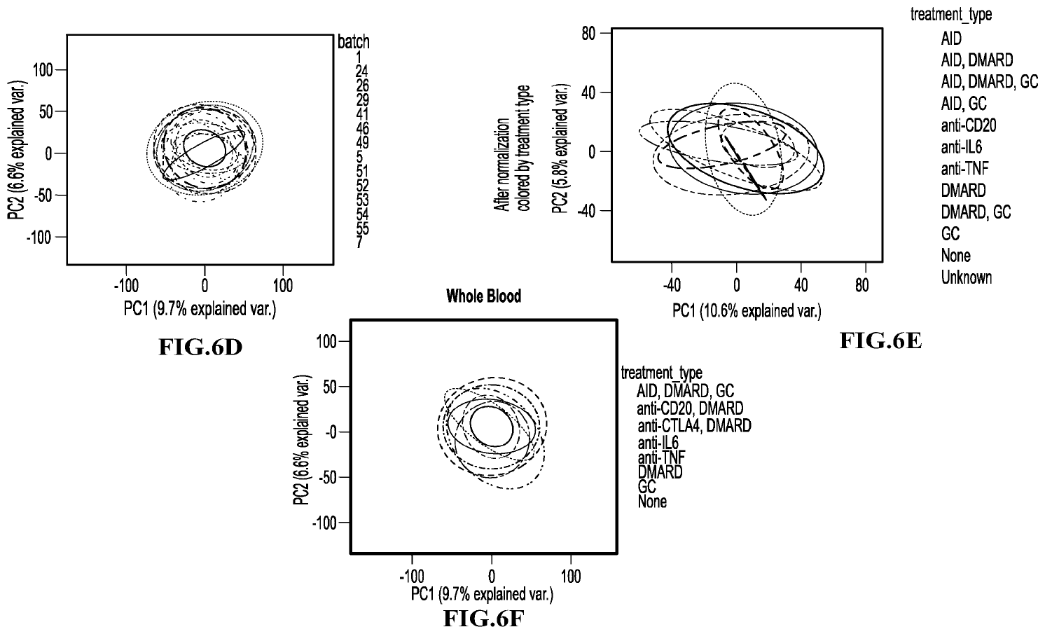

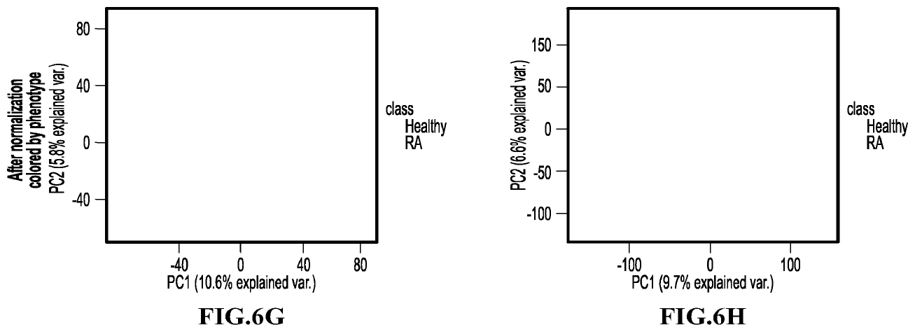

Raw data was downloaded and processed using R language version 3.6.5 and the Bioconductor packages SCAN, UPC, affy and limma. Processing steps included background correction, log 2-transformation, and intra-study quantile normalization (FIG. 1A). Next, we performed probe-gene mapping, data merging and normalization across batches with Combat within the R package sva. The dimensionality reduction plots before and after normalization are shown in FIG. 6. After merging studies, the total number of common genes was 11,057 in synovium and 14,596 in whole blood.

ii. Validation Data Collection and Processing

Five additional datasets from GEO were identified and downloaded: synovium microarray and RNA-seq, PBMC microarray and RNA-seq, whole blood microarray datasets (Table 1). Microarray data was processed as described above. RNA-seq data from GSE89408 were downloaded in a form of processed data of feature counts, which were normalized using the variance stabilizing transformation function vst( ) from the R package, DESeq2 (ref to DESeq2). RNA-seq data from GSE90081 were downloaded in a processed form of Fragments Per Kilobase Million (FPKM) counts, which were converted to Transcripts Per Kilobase Million (TPM) counts followed by log 2 transformation with 0.1 offset.

ii. Differential Gene Expression & Pathway Analysis

Differentially expressed genes were identified using a linear model from the R package limma. To account for factors related to gene expression, the imputed sex and treatment categories were used as covariates. Treatment types were categorized based on the drug class (Table 2). For 877 (40%) samples without sex annotations, sex was imputed using the average expression of Y chromosome genes. Significance for differential expression was defined using the cutoff of FDR p-value<0.05 and abs(FC)>1.2. Pathway analysis of differentially expressed genes was performed using the package clusterProfiler with the Reactome database as well as the gene list enrichment analysis tool ToppGene (https://toppgene.cchmc.org/).

TABLE 2
Treatment Classification

| Treatment category | What includes | Drugs | Functions |
|---|---|---|---|
| None | No treatment | — | — |
| DMARD | DMARD + NSARD | Gold, methotrexate, | MTX: Folic acid antagonist, |
| | | hydroxychloroquine, | HCQ: Antimalarial, |
| | | cyclosporine | CSN: Calcineurin inhibitor |
| AID | NSAID | sulfasalazine, celecoxib, | |
| | | azulfidine, COX-2 inhibitors | |
| GC | Corticosteroids | prednisolone | Glucocorticoid |
| anti-TNF | | infliximab, golimumab, | TNF alpha antagonist |
| | | etanercept, adalimumab, etc | |
| anti-CTLA4 | | abatacept | Binding to CD80/CD86, |
| | | | blocking T-cell co- |
| | | | stimulation |
| anti-CD20 | | rituximab | Binding to CD20 and depletion |
| | | | of CD20+ B cells |
| anti-IL1 | | anakinra | Binding to IL-1 type-1 |
| | | | receptor |
| anti-IL6 | | tocilizumab | Binding to soluble and |
| | | | membrane bound IL-6 |
| | | | receptor |
| Unknown | All indefinite | ? | — |
| | treatments | | |

iv. Cell Type Enrichment Analysis

In order to estimate the presence of certain cell types in a tissue, we leveraged the cell type enrichment analysis tool, xCell which computes enrichment scores for 64 immune and stromal cells based on gene expression data. We limited our analysis to 53 types of stromal, hematopoietic, and immune cells we expected to be present in blood and synovium. The cell types with a detection p-value greater than 0.2 taken as a medium across all samples in a tissue were filtered. Non-parametric Wilcoxon-Mann test with multiple testing correction with Benjamini-Hochberg approach (cut-off 0.05) was used to assess significantly enriched cell types in synovium and whole blood in RA compared to healthy control subjects. The effect size of each cell type was estimated by computing the ratio of the mean enrichment score in RA patients over mean score in healthy individuals.

v. Feature Selection Pipeline

The feature selection procedure is represented in FIG. 1B. First, for each tissue, data were split into training and testing sets in an 80:20 ratio with random sample selection and class distribution preservation using the function createDataPartiion0 from the R package caret. Within each training set, a set of significant genes was identified using limma FDR p-value<0.05. Pearson correlation was computed with the case-control status for each significant gene and those with r<0.25 were filtered out. For robustness and reducing gene redundancy, we computed gene pair-wise correlations and removed genes with correlation greater than 0.8. Next, we overlapped the gene sets from both tissues and filtered out any genes differentially expressed in opposite directions in synovium and blood. To monitor statistical significance of gene overlaps, we computed p-values using the hypergeometric test. To evaluate each gene performance in distinguishing RA from Healthy samples, we trained a logistic regression model per gene on a training set for each tissue and tested it on a testing set using area under receiver operating characteristic (AUROC) curve as a performance measure.

We repeated these steps 100 times to minimize bias of a random split into training and testing sets. From the resulting 100 gene sets, any gene that was found in each set was carried to the further analysis. The AUC performance of each gene was averaged, and its standard deviation was calculated. We then set the AUC threshold to ⅔ and applied this criterion to the testing results to identify the genes with the best performance, the feature selected genes.

vi. Feature Validation and RAScore

We used the five independent validation datasets to evaluate the feature selected genes. To evaluate and compare the value of the feature selected genes and the common DE genes in diagnostics, we proceeded with training machine learning models on the discovery blood data with these two gene sets and testing them on 5 validation sets. As some genes were not present in all validation sets, we reduced the gene sets to the genes that were found in all 5 validation sets. We used three machine learning algorithms, Logistic Regression, Elastic Net and Random Forest, for training classification models and AUROC for measuring their performance. We trained a Logistic Regression model for each feature selected gene individually on the discovery blood data and tested on the validation sets. AUROC was used as a performance measure. The genes with average AUC greater than 0.8 were selected. The selected genes were used to create the RAScore, computed by subtracting the geometric mean expression of the down-regulated genes from the geometric mean expression of the up-regulated genes.

Next, to recognize the clinical value of the selected genes and the RAScore, we identified datasets with samples that included values for DAS28, a measure of disease activity in RA. We computed the Pearson correlation coefficients of RAScore and expression levels of the feature selected genes with DAS28. Six datasets with both RA and Osteoarthritis (OA) samples (Table 1) were used to evaluate the ability of the RAScore to distinguish RA from OA. GSE74143 was used to test the difference in RAScore between RA sub-phenotypes with positive and negative Rheumatoid Factors. GSE45876 and GSE93272 were used to test the RAScore difference between treated and untreated RA patients. Additionally, we leveraged 10 datasets to test the ability of the RAScore to recognize polyarticular Juvenile Idiopathic Arthritis (polyJIA) (Table 1).

2. Results

i. Cross-Tissue Differential Expression and Pathway Analysis Reveals Significant Similarities on Gene and Pathway Levels

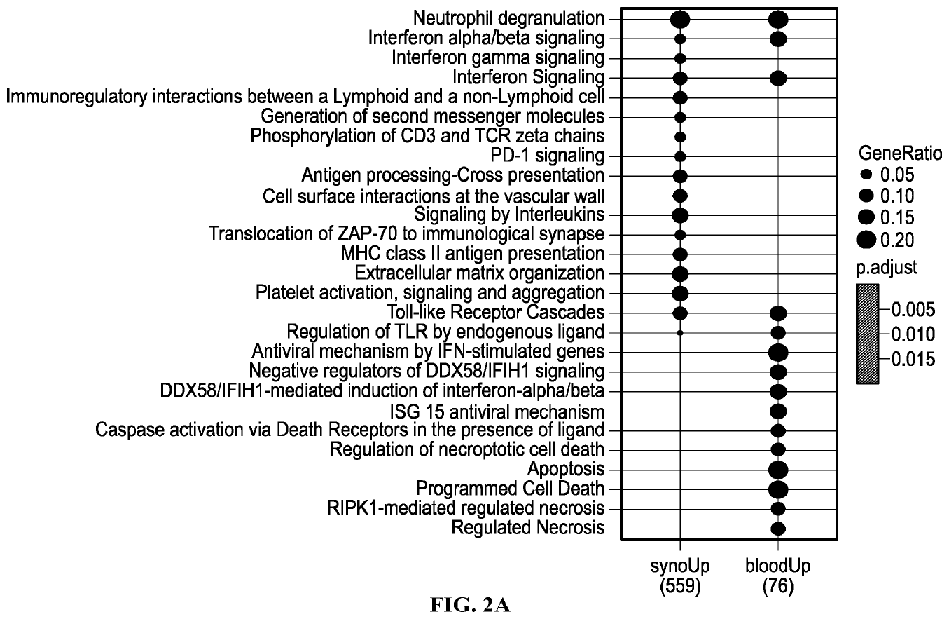

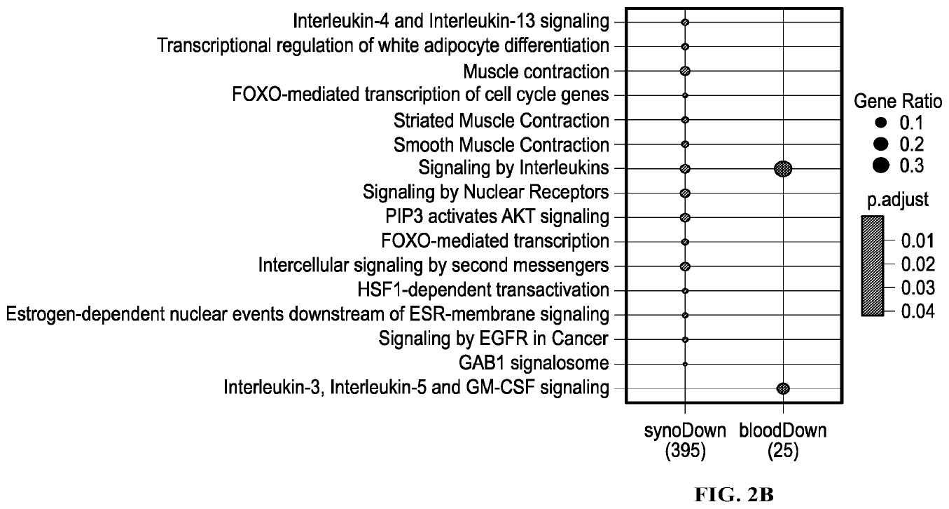

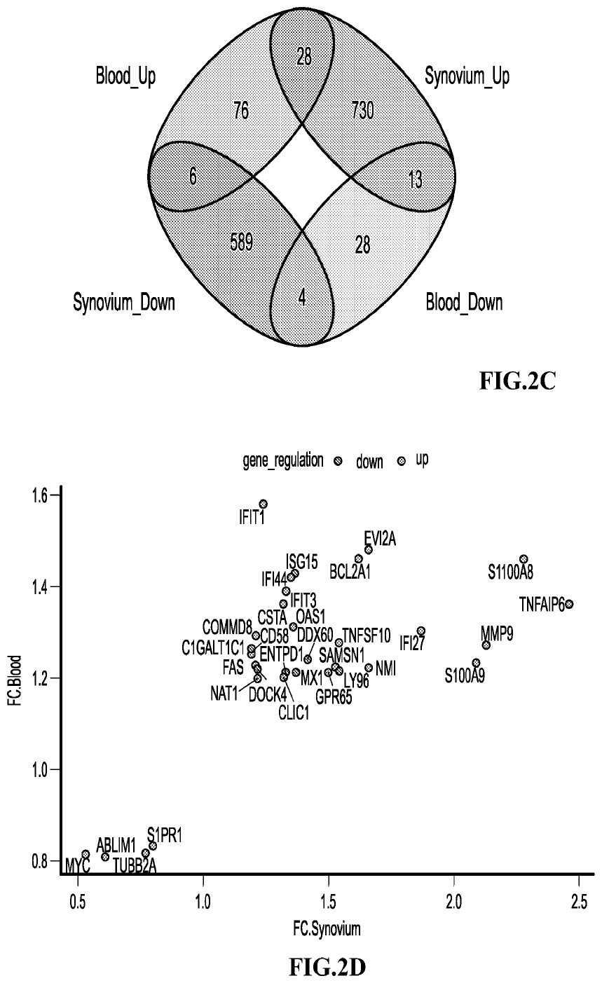

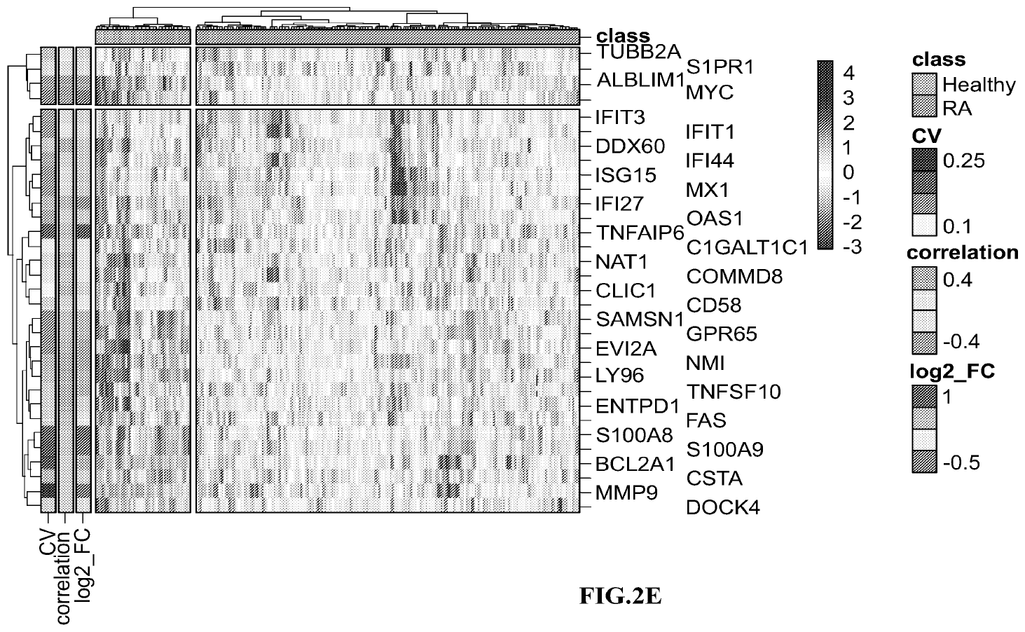

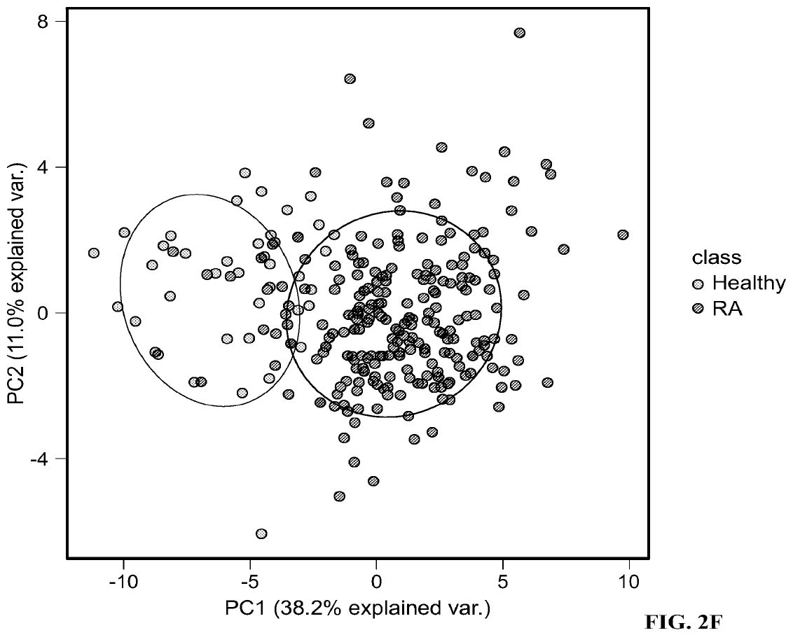

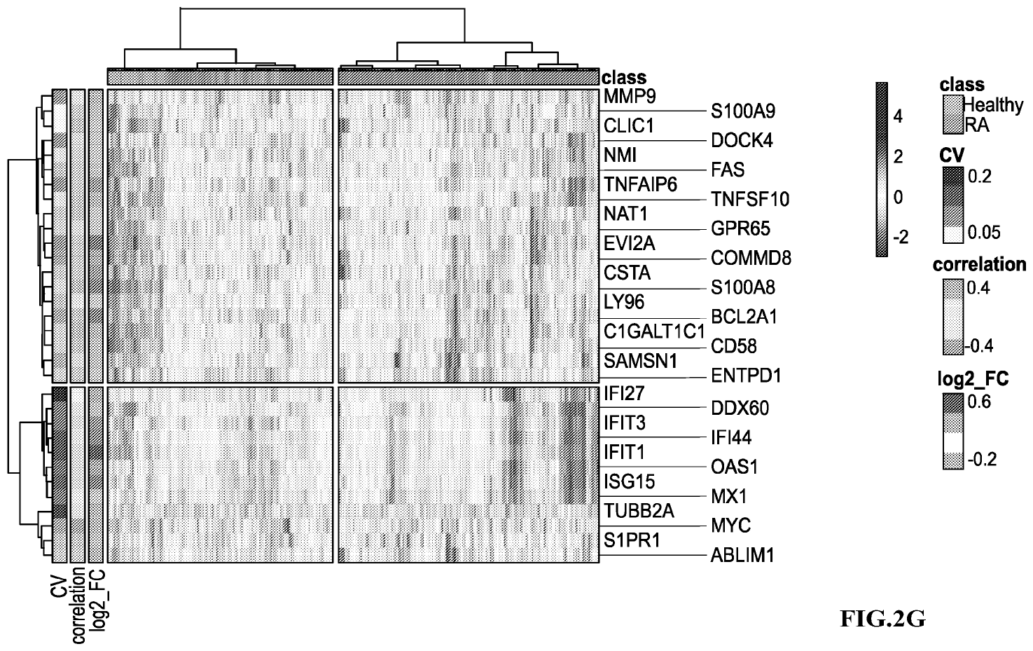

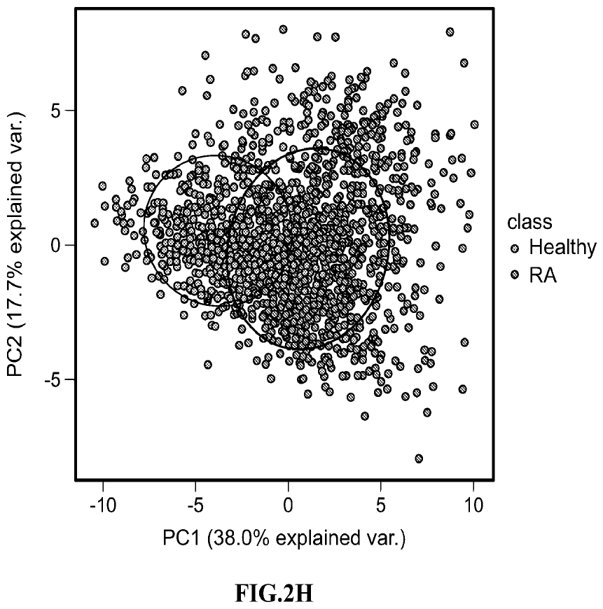

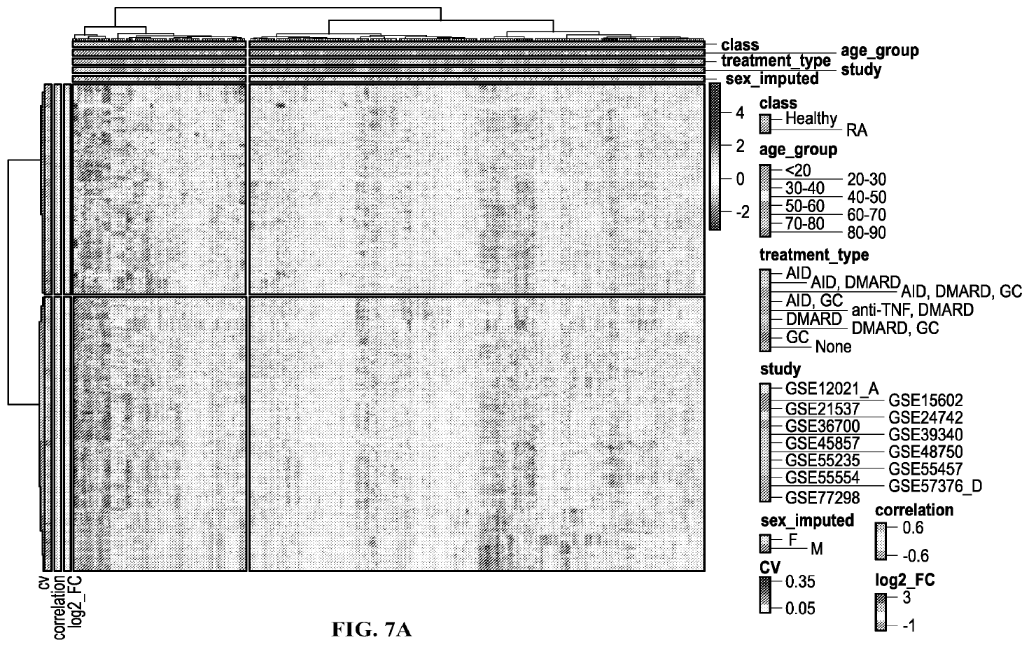

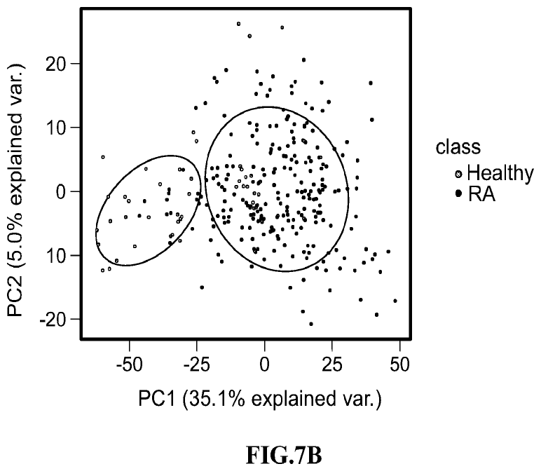

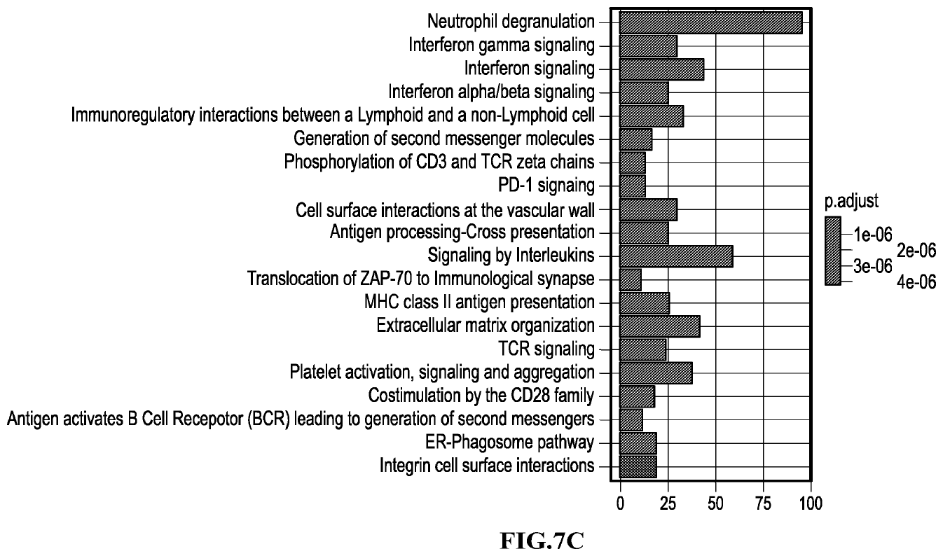

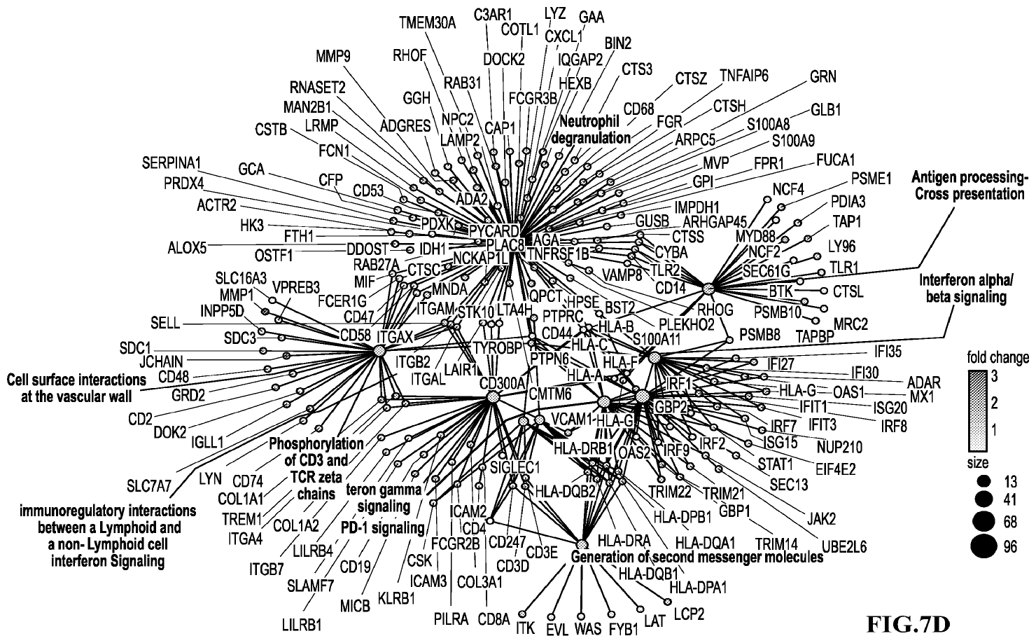

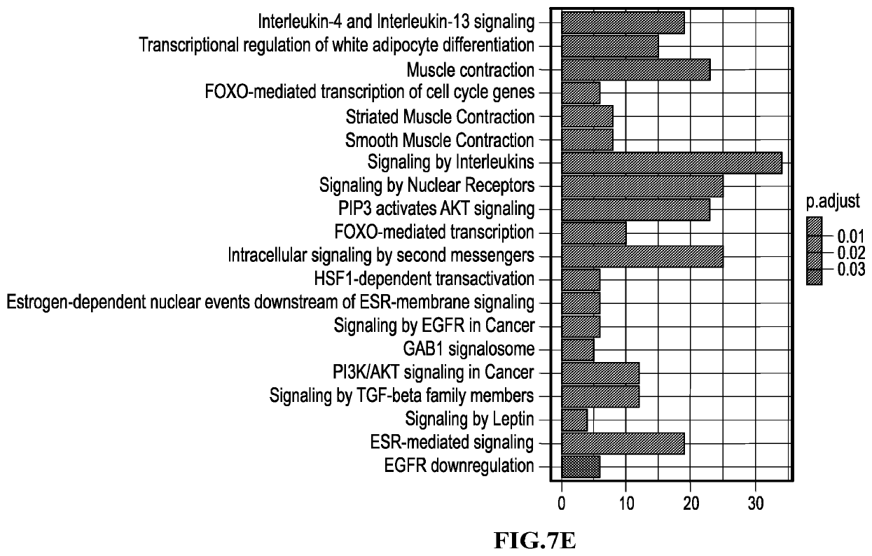

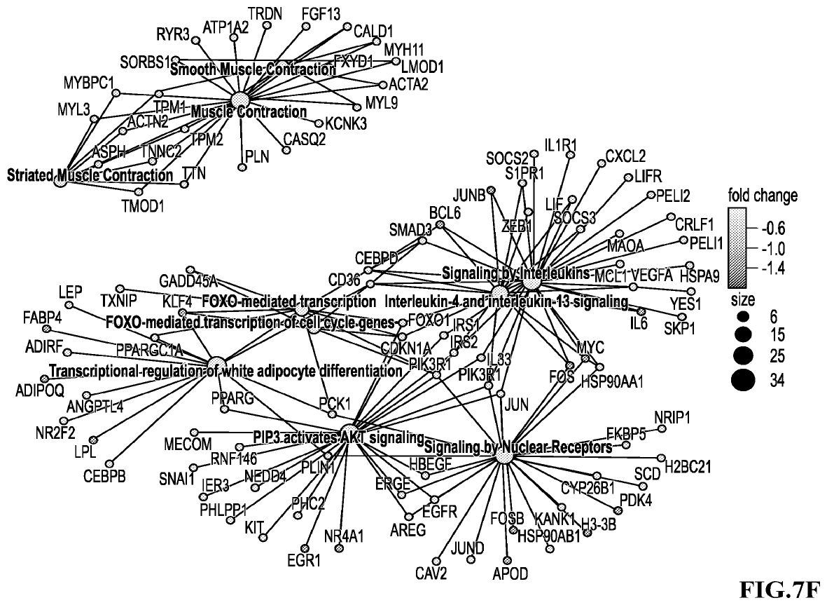

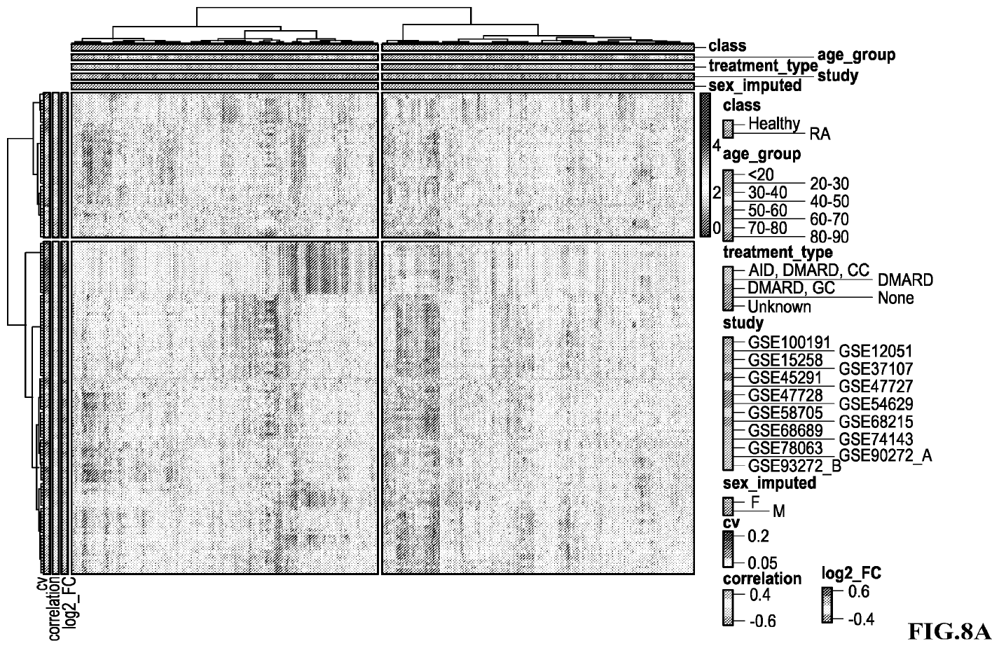

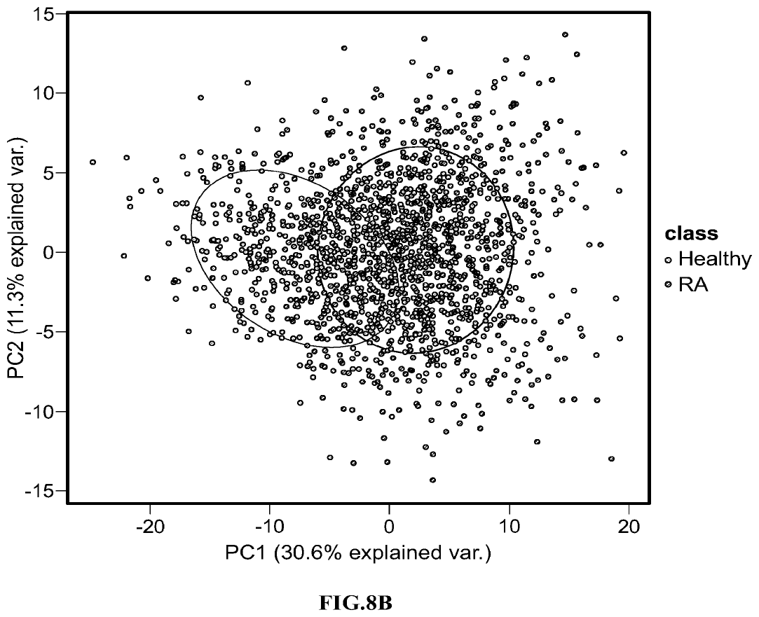

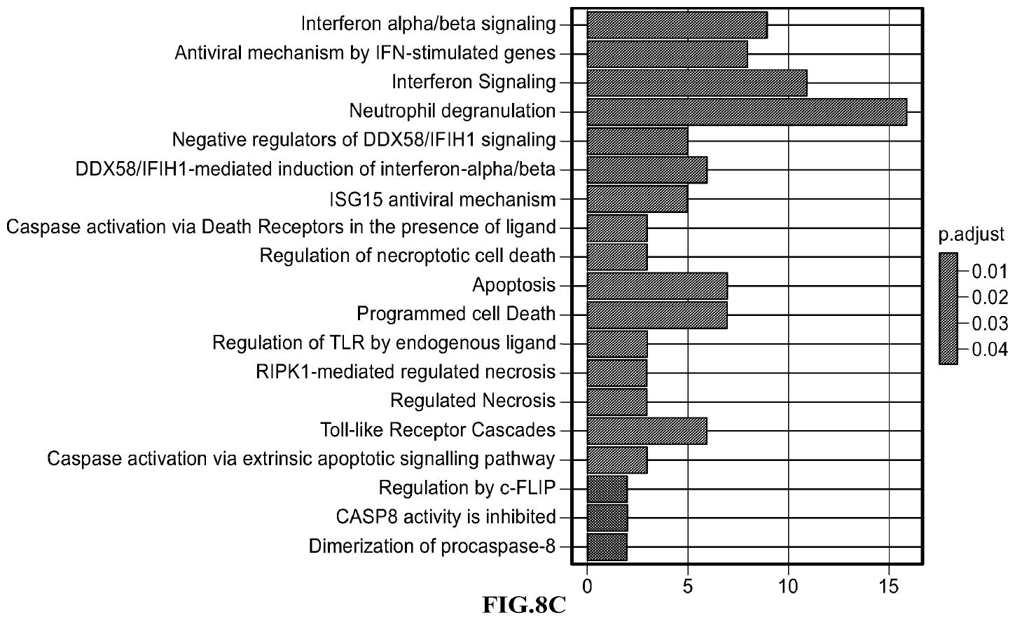

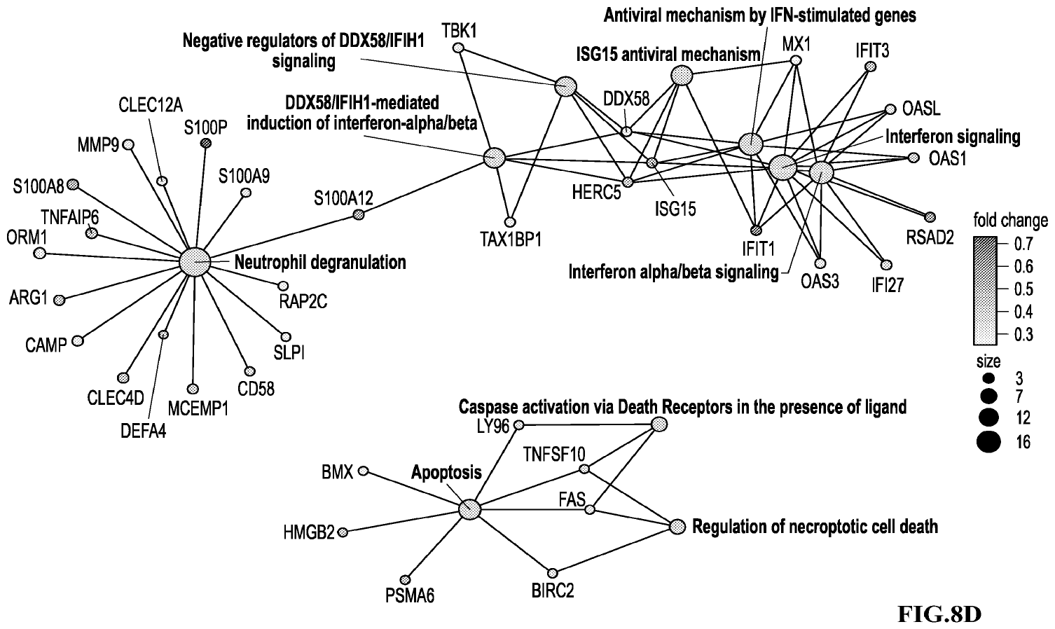

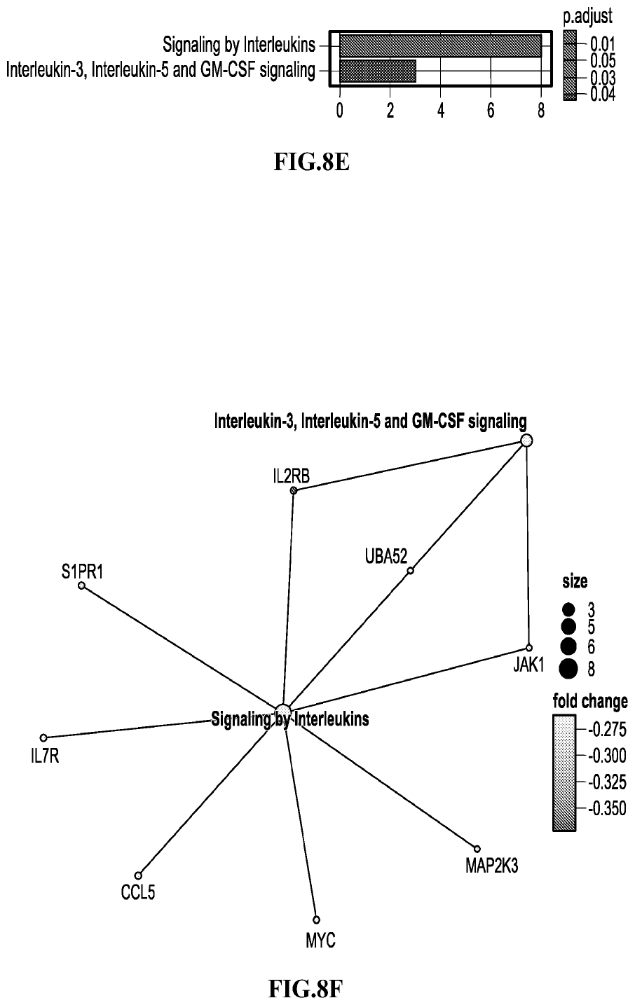

The differential gene expression analysis identified 1,370 genes with 771 up-regulated and 599 down-regulated genes in the synovium (FIG. 7A, FIG. 7B) and 155 genes with 110 up-regulated and 45 down-regulated genes in the blood (FIG. 8A, FIG. 8B). The pathway analysis revealed that in both tissues up-regulated genes shared enrichments in neutrophil degranulation, interferon alpha/beta signaling, toll-like receptor cascades, regulation of TLR by endogenous ligand, and caspase activation via extrinsic apoptotic signaling pathways (FIG. 2A, FIG. 7C, FIG. 7D, FIG. 8C, FIG. 8D, Table 3), while interferon gamma signaling, MHC class II antigen presentation, TCR signaling were specific for synovium and apoptosis, programmed cell death, antiviral mechanisms, DDX58/IFIH1-mediated induction of interferon-alpha/beta pathways were specific for blood (Table 4). The down-regulated genes were commonly involved only in signaling by interleukins pathway (FIG. 2B, FIG. 7E, FIG. 7F, FIG. 8E, FIG. 8F). However, the signaling by interleukins was also a common pathway with up-regulated genes in synovium coupled with enrichment in interleukin-5, interleukin-13 and GM-CSF signaling pathways. Many pathways were not shared suggesting different molecular mechanisms underlying in tissues. For example, the interleukin-4, interleukin-13 signaling, muscle contraction, FOXO-mediated transcription, and ESR-mediated signaling pathways were specific only for synovium (Table 3, Table 4).

TABLE 3
Significant Pathways - Synovium

| Description | geneID |
|---|---|
| Retinoid metabolism and transport | APOE/LRP1/APOB/AKR1B10/SDC4/RSP4/AXR1C1/LPL |
| Metabolism of fat-soluble vitamins | APOE/LRP1/APOB/AKR1B10/SDC4/RSP4/AXR1C1/LPL |
| Regulation of Insulin-like Growth Factor (IGF) transport | IGFBP2/APOE/SPARCL1/PENK/APOB/PNPLA2/KTN1/ |
| and uptake by Insulin-like Growth Factor Binding | CP/IGFBP6/IGFBP5/IL6/CCN1 |
| Proteins (IGFBPs) | |
| Transcriptional regulation of white adipocyte | PPARGC1A/LEP/ADIRF/PCK1/LPL/PLIN1/ |
| differentiation | KLF4/ADIPOQ/FABP4 |
| Interleukin-4 and Interleukin-13 signaling | ZEB1/FOXO1/VEGFA/LIF/SOCS3/IL6/MYC/ |
| | MAOA/JUNB/FOS |
| Post-translational protein phosphorylation | APOE/SPARCL1/PENK/APOB/PNPLA2/KTN1/CP/ |
| | IGFBP5/IL6/CON1 |
| Metabolism of vitamins and cofactors | ENPP1/APOE/SLC19A3/AOX1/LRP1/APOB/AXRIB10/ |
| | SDC4/RBP4/ACACB/AKR1C1/LPL/SLC19A2 |
| FOXO-mediated transcription of cell cycle genes | SMAD3/FOXO1/GADD45A/KLF4 |
| Visual phototransduction | METAP2/APOE/LRP1/APOB/AKR1B10/SDC4/RBP4/AKR1C1/LPL |
| FOXO-mediated transcription | SMAD3/PPARGC1A/TXNIP/FOXO1/GADD4SA/PCK1/KLF4 |
| Signaling by Leptin | LEP/SOCS3/IRS2 |
| HSF1-dependent transactivation | HSP90AB1/DNAJB1/HSPB8/CRYAB |
| Interleukin-6 family signaling | LIF/CRLF1/SOCS3/IL5 |
| Estrogen dependent nuclear events downstream of ESR- | AREG/EREG/HBEGF/FOS |
| membrane signaling | |
| Growth hormone receptor signaling | SOCS2/SOCS3/IRS2/GHR |
| GRB2 events in EGFR signaling | AREG/EREG/HBEGF |
| Phase I - Functionalization of compounds | CYP4F12/HSP90AB1/MARC1/CYP51A1/CYP26B1/ |
| | CYP4B1/MAOA/ADH1B |
| SHC1 eversts in EGFR signaling | AREG/EREG/HBEGF |
| FOXO-mediated transcription of oxidative stress, | SMAD3/PPARGC1A/FOXO1/PCK1 |
| metabolic and neuronal genes | |
| EGFR downregulation | AREG/SPRY2/EREG/HBEGF |
| GAB1 signalosome | AREG/EREG/MBEGF |
| Hyaluronan metabolism | HAS2/LYVE1/HAS1 |
| G alpha (i) signalling events | ADCY2/METAP2/APOE/PENK/LRP1/APOB/AKR1B10/CXCL3/ |
| | PRKAR2B/AGT/ACKR3/SDC4/NPY1R/CXCL2/RGS16/RBP4/ |
| | AXR1C1/LPL |
| Signaling by TGF-beta Receptor Complex | PARD3/SMAD3/TGFBR2/PPP1R15A/MYC/JUNB |
| Transcriptional regulation by RUNX3 | SMAD3/BRD2/TCF7L1/CTNNB1/TCF7L2/HES1/MYC |
| Signaling by PTKG | SFPQ/KHDRBS3/EREG/SOCS3/HBEGF |
| Signaling by Non-Receptor Tyrosine Kinases | SFPQ/KHDRBS3/EREG/SOCS3/HBEGF |
| Signaling by Nuclear Receptors | HSP90AB1/CYP26B1/AREG/NRIP1/JUND/H3F3B/FKBPS/ |
| | EREG/PDK4/MYC/HBEGF/FOS/FOSB |
| Signaling by TGF-beta family members | PARD3/SMAD3/BMP2/TGFBR2/PPP1R1SA/MYC/JUNB |
| Synthesis, secretion, and inactivation of Glucagon-like | CTNNB1/LEP/TCF7L2 |
| Peptide-1 (GLP-1) | |
| NOTCH4 Intracellular Domain Regulates Transcription | ACTA2/SMAD3/HES1 |
| mRNA 3′-end processing | CASC3/SRSF11/SRSF4/SRSF7/SRSF5 |
| Transcriptional regulation by the AP-2 (TFAP2) family of | APOE/KIT/VEGFA/MYC |
| transcription factors | |
| Signaling by Interleukins | PEL1/SMAD3/HSPA9/ZEB1/YES1/FOXO1/VEGFA/SOCS2/LIF/ |
| | IL1R1/CALF1/SOCS3/CXCL2/IRS2/IL6/MYC/MAOA/JUNB/FOS |
| Biological oxidations | CYP4F12/HSP90AB1/UGDH/MARC1/CYPS1A1/CYP26B1/ |
| | MAT2A/HPGDS/CYP4B1/MAOA/ADH1B |
| ESR-mediated signaling | HSP90AB1/AREG/NRI91/JUND/H3F3B/FKBP5/EREG/MYC/ |
| | HBEGF/FOS/FOSB |
| Incretin synthesis, secretion, and inactivation | CTNNB1/LEP/TCF7LX |
| Binding and Uptake of Ligands by Scavenger Receptors | APOE/LRP1/APOB/HBB |
| Deactivation of the beta-catenin transactivating complex | TCF7L1/SOX9/CTNNB1/TCF7L2 |
| Peptide hormone metabolism | CPA3/CTNNB1/AGT/LEP/TCF7L2/KLF4 |
| Signaling by EGFR in Cancer | AREG/EREG/HBEGF |
| RNA Polymerase II Transcription Termination | CASC3/SRSF11/SRSF4/SRSF7/SRSF5 |
| PI3K events in ERB84 signaling | EREG/HBEGF |
| Calcitonin-like ligand receptors | RAMP2/ADM |
| Regulation of FOXO transcriptional activity by acetylation | TXNIP/FOXO1 |
| Downregulation of TGF-beta receptor signaling | SMAD3/TGFBR2/PPP1R1SA |
| Interleukin-10 signaling | LIF/IL1R1/CXCL2/IL6 |
| Extracellular matrix organization | MMP14/ADAMTS5/BMP2/ITGA7/TPSAB1/NID1/SDC4/MFAP5/ |
| | LTBP4/DDR2/PCOLCE2/LAMA2/ADAMTS1 |
| Interleukin-6 signaling | SOCS3/IL6 |
| Metallothioneins bind metals | MT1M/MT1X |
| Signaling by EGFR | AREG/SPRY2/EREG/HBEGF |
| Transport of Mature mRNA derived from an Intron- | CASC3/SRSF11/SRSF4/SRSF7/SRSF5 |
| Containing Transcript | |
| Constitutive Signaling by Aberrant PI3K in Cancer | KIT/AREG/EREG/IRS2/HBEGF |
| PI3K/AKT Signaling in Cancer | KIT/AREG/FOXO1/EREG/IRS2/HBEGF |
| Eicosanoids | CYP4F12/CYP4B1 |
| Repression of WNT target genes | TCF7L1/TCF7L2 |
| Laminin interactions | ITGA7/NID1/LAMA2 |
| Plasma lipoprotein remodeling | APOE/APOB/LPL |
| alpha-linolenic (omega3) and linoleic (omega6) acid | FADS1/ACSL1 |
| metabolism | |
| alpha-linolenic acid (ALA) metabolism | FADS1/ACSL1 |
| Scavenging of heme from plasma | LRP1/HBB |
| ERBB2 Activates PTK6 Signaling | EREG/HBEGF |
| TGF-beta receptor signaling activates SMADs | SMAD3/TGFBR2/PPP1R1SA |
| SMAD2/SMAD3: SMAD4 heterotrimer regulates | SMAD3/MYC/JUNB |
| transcription | |
| Regulation of cholesterol biosynthesis by SRESP (SREBF) | CYP51A1/RAN/SCD/ACACB |
| HSP90 chaperone cycle for steroid hormone receptors | HSP90AB1/NR3C2/DNAJB1/FKBPS |
| (SHR) | |
| SHC1 events in ERBB4 signaling | EREG/HBEGF |
| Attenuation phase | HSP90AB1/DNAJB1 |
| Response to metal ions | MT1M/MT1X |
| Negative regulation of the PI3K/AKT network | PHLPP1/KIT/AREG/EREG/IRS2/HBEGF |
| Transport of Mature Transcript to Cytoplasm | CASC3/SRSF11/SRSF4/SRSF7/SASF8 |
| Fatty acids | CYPAF12/CYP4B1 |
| Negative regulation of TCF-dependent signaling by WNT | WIF1/SFRP1 |
| ligand antagonists | |
| ERBB2 Regulates Cell Motility | EREG/HBEGF |
| The NLRP3 inflammasomne | HSP90AB1/TXNIP |
| TFAP2 (AP-2) family regulates transcription of growth | KIT/VEGFA |
| factors and their receptors | |
| Regulation of KIT signaling | YES1/KIT |
| GRB2 events in ERBB2 signaling | EREG/HBEGF |
| PI3K events in ERBB2 signaling | EREG/HBEGF |
| TGF-beta receptor signaling in EMT (epithelial to | PARD3/TGFBR2 |
| mesenchymal transition) | |
| Ca2+ pathway | WNT11/TCF7L1/CTNNB1/TCF7L2 |
| Fatty acyl-CoA biosynthesis | HACD1/SCD/ACSL1 |
| Fatty acid metabolism | FADS1/HACD1/PHYH/SCD/HPGDS/ACSL1/ACACB/CYP4B1 |
| Cellular response to heat stress | HSP90AB1/HSPA9/DNAJB1/HSPB8/CRYAB |
| Molecules associated with elastic fibres | BMP2/MFAP5/LTBP4 |
| PKA activation in glucagon signalling | ADCY2/PRKAR2B |
| IL-6-type cytokine receptor ligand interactions | LIF/CRLF1 |
| Formation of the beta-catenin: TCF transactivating | TCF7L1/CTNNB1/TCF7L2/H3F3B/MYC |
| complex | |
| Estrogen-dependent gene expression | HSP90AB1/NRIP1/JUND/H3F3B/MYC/FOS/FOSB |
| Formation of Fibrin Clot (Clotting Cascade) | SERPINAS/THBD/TFPI |
| Transcriptional regulation by RUNX2 | PPARGC1A/YES1/BMP2/SOX9/ITGBL1/HES1 |
| mRNA Splicing - Major Pathway | CASC3/SRSF11/SRSF4/TRA2B/HNRNPA0/SRSF7/SRRM2/SRSF5 |
| Metabolism of Angiotensinogen to Angiotensins | CPA3/AGT |
| Plasma lipoprotein assembly | APOE/APOB |
| Smooth Muscle Contraction | ACTA2/SORBS3/LMOD1 |
| Cytochrome P450 - arranged by substrate type | CYP4F12/CYP51A1/CYP26B1/CYP4B1 |
| Glycosaminoglycan metabolism | H53ST2/UST/HA52/SDC4/LYVE1/HAS1 |
| Diseases of signal transduction | PEBP1/SMAD3/KIT/AREG/TGFBR2/FOXO1/CTNNB1/TCF7L2/ |
| | HES1/EREG/IRS2/RBP4/MYC/HBEGF |
| PKA activation | ADCY2/PRKAR2B |
| Scavenging by Class A Receptors | APOE/APOB |
| Synthesis, secretion, and deacylation of Ghrelin | LEP/KLF4 |
| Activation of gene expression by SREBF (SRESP) | CYP51A1/SCD/ACACB |
| Cellular responses to stress | HSP90AB1/HSPA9/ETS2/H1F0/NR3C2/PRDX6/DNAJB1/ |
| | VEGFA/HSPB8/H3F3B/FKBPS/IL6/CRYAB/GPX3/FOS |
| Peptide Kgand-binding receptors | ECE1/PENK/CXCL3/AGT/ACKR3/ACKR1/NPY1R/CXCL2 |
| mRNA Splicing | CASC3/SRSF11/SRSF4/TRA2B/HNRMPA0/SRSF7/SRRM2/SRSF5 |
| Signaling by Hippo | SAV1/AMOTL2 |
| Inflammasomes | HSP90AB1/TXNIP |
| Circadian Clock | PPARGC1A/NRIP1/PER1/NFIL3 |
| MAPK family signaling cascades | PEBP1/KIT/AREG/FOXO1/DNAJB1/EREG/IRS2/IL6/ |
| | DUSP1/MYC/HBEGF |
| Transcriptional activity of SMAD2/SMAD3; SMAD4 | SMAD3/MYC/JUNB |
| heterotrimer | |
| Metabolism of carbohydrates | ENO1/AKR1B1/GBE1/HS3ST2/PFKM/UST/HAS2/SDC4/ |
| | LYVE1/PCK1/HAS1 |
| PKA-mediated phosphorylation of CREB | ADCY2/PRKAR2B |
| TP53 regulates transcription of additional cell cycle | RGCC/BTG2 |
| genes whose exact role in the p53 pathway remain | |
| uncertain | |
| Elastic fibre formation | BMP2/MFAP5/LTBP4 |
| SHC1 events in ERBB2 signaling | EREG/HBEGF |
| Common Pathway of Fibrin Clot Formation | SERPINA5/THBD |
| PISP, PP2A and IER3 Regulate PI3K/AKT Signaling | KIT/AREG/EREG/IRS2/HBEGF |
| TCF dependent signaling in response to WNT | |
| RAF-independent MAPK1/3 activation | IL6/DUSP3 |
| Intracellular signaling by second messengers | ADCY2/SNAI1/PHLPP1/KIT/AREG/FOXO1/PRKAR2B/ |
| | EREG/IRS2/HBEGF/EGR1 |
| Chemokine receptors bind chemokines | CXCL3/ACKR3/CXCL2 |
| Non-genomic estrogen signaling | AREG/EREG/HBEGF/FOS |
| TP53 Regulates Transcription of Cell Cycle Genes | RGCC/BTG2/GADD45A |
| Triglyceride catabolism | PLIN1/FABP4 |
| Synthesis of very long-chain fatty acyl-CoAs | HACD1/ACSL1 |
| RUNX2 regulates osteoblast differentiation | YES1/HES1 |
| Signaling by ERBB2 | YES1/EREG/HBEGF |
| Musde contraction | ACTA2/SORBS3/RYR3/TNNC2/KCNK3/LMOD1/ATP1A2/TMOD1 |
| Cathrin-mediated endocytosis | TRIP10/APOB/FNBP1L/AREG/EREG/HBEGF |
| Nuclear signaling by ERBB4 | EREG/NBEGF |
| PPARA activates gene expression | FADS1/PPARGC1A/PLIN2/AGT/ACSL1 |
| Metabolism of steroids | AKR1B1/CYP51A1/RAN/SCD/ACACB/AKR1C1 |
| Sulfur amino acid metabolism | BHMT2/CDO1 |
| Regulation of lipid metabolism by Peroxisome | FADS1/PPARGC1A/PLIN2/AGT/ACSL1 |
| proliferator-activated receptor alpha (PPARalpha) | |
| Downregulation of ERBB2 signaling | EREG/HBEGE |
| Complement cascade | C6/CFD/C7 |
| Surfactant metabolism | CCDC59/LMCD1 |
| Non-integrin membrane ECM interactions | SDC4/DDR2/LAMA2 |
| Metabolism of water-soluble vitamins and cofactors | ENPP1/SLC19A3/AOX1/ACACB/SLC19A2 |
| HS-GAG biosynthesis | HS3ST2/SDC4 |
| Uptake and actions of bacterial toxins | HSP90AB1/HBEGF |
| PIP3 activates AKT signaling | SNAI1/PHLPP1/KIT/AREG/FOXO1/EREG/IRS2/ |
| | HBEGF/EGR1 |
| RUNX2 regulates bone development | YES1/HES1 |
| Activation of Matrix Metalloproteinases | MMP14/TPSAB1 |
| Glucagon signaling in metabolic regulation | ADCY2/PRKAR2B |
| Plasma lipoprotein clearance | APOE/APOB |
| Class B/2 (Secretin family receptors) | WNT11/RAMP2/FZD10/ADM |
| Signaling by WNT in cancer | CTNNB1/TCF7L2 |
| Gluconeogenesis | ENO1/PCK1 |
| Calmodulin induced events | ADCY2/PRKAR2B |
| CaM pathway | ADCY2/PRKAR2B |
| Interleukin-7 signaling | SOCS2/IRS2 |
| Striated Muscle Contraction | TNNC2/TMOD1 |
| Metabolic disorders of biological oxidation enzymes | CYP25B1/MAOA |
| Processing of Capped Intron-Containing Pre-mRNA | CASC3/SRSF11/SRSF4/TRA2B/HNRNPA0/ |
| | SRSF7/SRRM2/SRSF5 |
| MARK3 (ERK1) activation | IL6 |
| Neurotransmitter clearance | MAOA |
| Adenylate cyclase activating pathway | ADCY2 |
| Thyroxine biosynthesis | DUOX2 |
| Activation of PPARGC1A (PGC-1alpha) by | PPARGC1A |
| phosphorylation | |
| Abacavir transport and metabolism | PCX1 |
| Activation of the AP-1 family of transcription factors | FOS |
| Signal attenuation | IRS2 |
| HDL remodeling | APOE |
| Detoxification of Reactive Oxygen Species | PRDX5/GPX3 |
| Defective B3GALTL causes Peters-plus syndrome (PpS) | ADAMTS5/ADAMTS1 |
| Triglyceride metabolism | PLIN1/FABP4 |
| Cell surface interactions at the vascular wall | YES1/APOB/SDC4/THBD/TSPAN7 |
| Plasma lipoprotein assembly, remodeling, and clearance | APOE/APOB/LPL |
| Ca-dependent events | ADCY2/PRKAR2B |
| O-glycosylation of TSR domain-containing proteins | ADAMTS5/ADAMTS1 |
| Degradation of the extracellular matrix | MMP14/ADAMTS5/TPSAB1/NID1/ADAMTS1 |
| Regulation of gene expression by Hypoxia-inducible | VEGFA |
| Factor | |
| Biotin transport and metabolism | ACACB |
| Dermatan sulfate biosynthesis | UST |
| Apoptotic cleavage of cell adhesion proteins | CTNNB1 |
| RHO GTPases activate KTN1 | KIN1 |
| Phenylalanine and tyrosine catabolismo | FAH |
| Interleukin-27 signaling | CRLF1 |
| Regulation of localization of FOXO transcription factors | FOXO1 |
| Cargo recognition for clathrin-mediated endocytosis | APOB/AREG/EREG/HBEGF |
| Synthesis of PA | GPD1L/GPD1 |
| Negative regulation of MAPK pathway | PEBP1/DUSP1 |
| Signaling by WNT | LGR4/WIF1/WNT11/TCF7L1/SOX9/CTNNB1/ |
| | TCF7L2/H3F3B/MYC/SFRP1 |
| MAPK1/MAPK3 signaling | PEBP1/KIT/AREG/EREG/IRS2/IL6/DUSP1/HBEGF |
| Integration of energy metabolism | ADCY2/PRKAR2B/ACACB/ADIPOQ |
| Tandem pore domain potassium channels | KCNK3 |
| Pregnenolone biosynthesis | AKR1B1 |
| FCGR activation | YES1 |
| PECAM1 interactions | YES1 |
| Miscellaneous substrates | CYP4B1 |
| Hyaluronan uptake and degradation | LYVE1 |
| NOTCH2 intracellular domain regulates transcription | HES1 |
| HSF1 activation | HSP90AB1 |
| Lysine catabolism | AASS |
| Ethanol oxidation | ADH1B |
| Erythropoietin activates Phosphoinositide-3-kinase | IRS2 |
| (PI3K) | |
| DAG and 193 signaling | ADCY2/PRKAR2B |
| Regulation of beta-cell development | FOXO1/HES1 |
| EPHB-mediated forward signaling | YES1/EFNB2 |
| Erythrocytes take up carbon dioxide and release oxygen | HBB |
| Mitochondrial iron-sulfur cluster biogenesis | ISCA1 |
| Apoptosis induced DNA fragmentation | H1F0 |
| O2/CO2 exchange in erythrocytes | HBB |
| Signaling by Activin | SMAD3 |
| Signaling by SCF-KIT | YES1/KIT |
| Vasopressin regulates renal water homeostasis via | ADCY2/PRKAR2B |
| Aquaporins | |
| Signaling by Retinoic Acid | CYP26B1/PDK4 |
| Signaling by Receptor Tyrosine Kinases | RBFOX2/THBS4/YES1/KIT/AREG/CTNNB1/CILP/VEGFA/SPRY2/EREG/ |
| | LAMA2/IRS2/HBEGF |
| Methylation | MAT2A |
| Degradation of cysteine and homocysteine | CDO1 |
| Adenylate cyclase inhibitory pathway | ADCY2 |
| Membrane binding and targetting of GAG proteins | UBAP1 |
| Synthesis And Processing Of GAG, GAGPOL Polyproteins | UBAP1 |
| Synthesis of IP2, IP, and Ins in the cytosol | INPP5A |
| Synthesis of bile acids and bile salts via 24- | AKR1C1 |
| hydroxycholesterol | |
| Import of palmitoyl-CoA into the mitochondrial matrix | ACACB |
| Retinoid cycle disease events | RBP4 |
| Diseases associated with visual transduction | RBP4 |
| Defective EXT2 causes exostoses 2 | SDC4 |
| Defective EXT1 causes exostoses 1, TRP52 and CHDS | SDC4 |
| CREB1 phosphorylation through the activation of | PRKAR2B |
| Adenylate Cyclase | |
| Regulation of IFNG signaling | SOCS3 |
| RUNX3 regulates NOTCH signaling | HES1 |
| Erythropoietin activates RAS | IRS2 |
| Signaling by ERBB4 | EREG/HBEGF |
| SUMOylation of transcription cofactors | PPARGC1A/NRIP1 |
| Histidine, lysine, phenylalanine, tyrosine, proline and | AASS/FAH |
| tryptophan catabolism | |
| Prolactin receptor signaling | GHR |
| Synthesis of bile acids and bile salts via 27- | AKR1C1 |
| hydroxycholesterol | |
| Synthesis of Prostaglandins (PG) and Thromboxanes (TX) | HPGDS |
| phosphorylation site mutants of CTNNB1 are not | CTNNB1 |
| targeted to the proteasome by the destruction complex | |
| Misspliced GSK3beta mutants stabilize beta-catenin | CTNNB1 |
| S33 mutants of beta-catenin aren't phosphorylated | CTNNB1 |
| S37 mutants of beta-catenin aren't phosphorylated | CTNNB1 |
| S45 mutants of beta-catenin aren't phosphorylated | CTNNB1 |
| T41 mutants of beta-catenin aren't phosphorylated | CTNNB1 |
| IRAK1 recruits IKK complex | PELI1 |
| IRAK1 recruits IKK complex upon TLR7/8 or 9 stimulation | PELI1 |
| Degradation of beta catenin by the destruction complex | TCF7L1/CTNNE1/TCF7LZ |
| Signaling by NOTCH4 | ACTA2/SMAD3/HES1 |
| NOTCH1 Intracelular Domain Regulates Transcription | HES1/MYC |
| Regulation of Complement cascade | C6/C7 |
| G alpha (z) signalling events | ADCY2/RGS16 |
| Translesion synthesis by REV1 | REV3L |
| Spry regulation of FGF signaling | SPRYZ |
| Assembly Of The HIV Virion | UBAPI |
| Regulation of pyruvate dehydrogenase (PDH) complex | PDX4 |
| Regulation of gene expression in late stage (branching | HES1 |
| morphogenesis) pancreatic bud precursor cells | |
| Formation of Senescence-Associated Heterochromatin | H1F0 |
| Foci (SAHF) | |
| Glycogen storage diseases | GBE1 |
| Glycogen synthesis | GBE1 |
| Sema3A PAK dependent Axon repulsion | HSP90AB1 |
| cGMP effects | PDE2A |
| POXO-mediated transcription of cell death genes | FOXO1 |
| Transport of bile salts and organic acids, metal ions and | SLC47A1/SLC16A7/CP |
| amine compounds | |
| Fcgamma receptor (FCGR) dependent phagocytosis | HSP90AB1/MYH2/YES1 |
| Chondroitin sulfate/dermatan sulfate metabolism | UST/SDC4 |
| Acyl chain remodeling of PI | PLAAT3 |
| Beta-catenin phosphorylation cascade | CTNNB1 |
| Vitamin B5 (pantothenate) metabolism | ENPP1 |
| Trafficking of GluR2-containing AMPA receptors | TSPAN7 |
| Platelet sensitization by LDL | APOB |
| Butyrate Response Factor 1 (BRF1) binds and destabilizes | ZFP36L1 |
| mRNA | |
| Tristetraprolin (TTP, ZFP36) binds and destabilizes mRNA | ZFP36 |
| Translesion synthesis by POLK | REV3L |
| Translesion synthesis by POLI | REV3L |
| MECP2 regulates neuronal receptors and channels | FKBPS |
| EPH-ephrin mediated repulsion of cells | YES1/EFNB2 |
| Aquaporin-mediated transport | ADCY2/PRKAR2B |
| Apoptotic execution phase | H1F0/CTNNB1 |
| MAPK6/MAPK4 signaling | FOXO1/DNAJB1/MYC |
| Norepinephrine Neurotransmitter Release Cycle | MAOA |
| Amine-derived hormones | DUOX2 |
| Cell-extracellular matrix interactions | FERMT2 |
| Activation of SMO | GAS1 |
| TP53 Regulates Transcription of Genes Involved in G2 | GADD45A |
| Cell Cycle Arrest | |
| Gastrin-CREB signalling pathway via PKC and MAPK | HBEGF |
| Assembly of active LPL and LIPC lipase complexes | LPL |
| Platelet degranulation | CDC37L1/VEGFA/TIMP3/CFD |
| Glycerophospholipid biosynthesis | GPD1L/PNPLA2/PLAAT3/GPD1 |
| Cell junction organization | PARD3/CTNNB1/FERMT2 |
| Glucose metabolism | ENO1/PFKM/PCK1 |
| PLC beta mediated events | ADCY2/PAKAR2B |
| Signaling by Type 1 Insulin-like Growth Factor 1 Receptor | CILP/IRS2 |
| (IGF1R) | |
| Metabolism of amino acids and derivatives | SAT1/GLUL/RPL22/AASS/NQO1/DUOX2/BHMT2/FAH/ |
| | CDO1/RPS4Y1 |
| RAF/MAP kinase cascade | PEBP1/KIT/AREG/EREG/IRS2/DUSP1/HBEGF |
| Transcription of E2F targets under negative control by | MYC |
| DREAM complex | |
| Ephrin signaling | EFNB2 |
| Phase 4 - resting membrane potential | KCNK3 |
| Regulation of TLR by endogenous ligand | APOB |
| LDL clearance | APOB |
| G-protein mediated events | ADCY2/PRAKAR2B |
| Heparan sulfate/heparin (N5-GAG) metabolism | HS3ST2/SDC4 |
| Nucleotide-binding domain, leucine rich repeat | HSP90AB1/TXNIP |
| containing receptor (NLR) signaling pathways | |
| GPCR ligand binding | ECE1/PENK/WNT11/CXCL3/RAMP2/AGT/ACKR3/ |
| | FZD10/ACKR1/NPY1R/CXCL2/ADM |
| Ion homeostasis | RYR3/ATP1A2 |
| Signaling by NODAL | SMAD3 |
| Defective B4GALT7 causes EDS, progeroid type | SDC4 |
| Defective B3GAT3 causes JDSSDHD | SDC4 |
| Defective B3GALT6 causes EDSP2 and SEMDIL1 | SDC4 |
| RHO GTPases activate CIT | RHOB |
| RHO GTPases Activate ROCKs | RHOB |
| Listeria monocytogenes entry into host cells | CTNNB1 |
| Response to elevated platelet cytosolic Ca2+ | CDC37L1/VEGFA/TIMP3/CFD |
| Interleukin-12 family signaling | HSPA9/CRLF1 |
| Ion transport by P-type ATPases | CUTC/ATP1A2 |
| Regulation of gene expression in beta cells | FOXO1 |
| Synthesis of Leukotrienes (LT) and Eoxins (EX) | CYP4B1 |
| CTLA4 inhibitory signaling | YES1 |
| Sema4D induced cell migration and growth-cone | RHOB |
| collapse | |
| Regulation of FZD by ubiquitination | LGR4 |
| VEGFR2 mediated cell proliferation | VEGFA |
| Interleukin-37 signaling | SMAD3 |
| Signaling by NOTCH1 PEST Domain Mutants in Cancer | HES1/MYC |
| Signaling by NOTCHI1in Cancer | HES1/MYC |
| Constitutive Signaling by NOTCH1 PEST Domain Mutants | HES1/MYC |
| Signaling by NOTCH1 HD + PEST Domain Mutants in | HES1/MYC |
| Cancer | |
| Constitutive Signaling by NOTCH1 HD + PEST Domain | HES1/MYC |
| Mutants | |
| Iron uptake and transport | CYBRD1/CP |
| Arachidonic acid metabolism | HPGDS/CYP4B1 |
| Rho GTPase cycle | NET1/TRIP10/ARHGAP29/RHOB |
| Intrinsic Pathway of Fibrin Clot Formation | SERPINA5 |
| RS-GAG degradation | SDC4 |
| Nitric oxide stimulates guanylate cyclase | PDE2A |
| RA biosynthesis pathway | CYP26B1 |
| Digestion | PIR |
| Regulation of signaling by CBL | YES1 |
| Regulation of actin dynamics for phagocytic cup | HSP90AB1/MYH2 |
| formation | |
| Regulation of PTEN gene transcription | SNAI1/EGR1 |
| Acyl chain remodelling of PS | PLAAT3 |
| Initial triggering of complement | CFD |
| Downregulation of SMAD2/3: SMAD4 transcriptional | SMAD3 |
| activity | |
| The canonical retinoid cycle in rods (twilight vision) | RBP4 |
| RNA Polymerase III Transcription Termination | NFIB |
| Synthesis of bile acids and bile salts via 7alpha- | AKR1C1 |
| hydroxycholesterol | |
| MicroRNA (miRNA) biogenesis | RAN |
| Transcriptional regulation of pluripotent stem cells | KLF4 |
| Synthesis of substrates in N-glycan biosythesis | GFPT2/UAP1 |
| Peroxisomal protein import | PEX5/PHYH |
| Diseases of metabolism | CYP26B1/GBE1/MAOA |
| Beta-catenin independent WNT signaling | WNT11/TCF7L1/CTNNB1/TCF7L2 |
| Cell-cell junction organization | PARD3/CTNNB1 |
| Glucuronidation | UGDH |
| Cholesterol biosynthesis | CYP51A1 |
| Sema4D in semaphorin signaling | RHOB |
| Constitutive Signaling by AKT1 E37K in Cancer | FOXO1 |
| Signaling by Erythropoietin | IRS2 |
| NOTCH3 Intracellular Domain Regulates Transcription | HES1 |
| Semaphorin interactions | HSP90AB1/RHOB |
| DARPP-32 events | PAKAB2B |
| A tetrasaccharide linker sequence is required for GAG | SDCA |
| synthesis | |
| WNT ligand biogenesis and trafficking | WNT11 |
| Metal ion SLC transporters | CP |
| Resolution of D-loop Structures through Synthesis- | XRCC2 |
| Dependent Strand Annealing (SDSA) | |
| FGFR2 alternative splicing | RBFOX2 |
| Regulation of IFNA signaling | SOCS3 |
| Phase II - Conjugation of compounds | UGDH/MAT2A/HPGDS |
| G0 and Early G1 | MYC |
| Endogenous sterols | CYP51A1 |
| Syndecan interactions | SDC4 |
| Digestion and absorption | PIR |
| Diseases associated with O-glycosylation of proteins | ADAMTS5/ADAMTS1 |
| Senescence-Associated Secretory Phenotype (SASP) | H3F3B/IL6/FOS |
| Cellular Senescence | ETS2/H1F0/H3F3B/IL6/FOS |
| Regulation of HSF1-mediated heat shock response | HSPA9/DNAJB1 |
| Interferon alpha/beta signaling | SOC53/EGR1 |
| Class A/1 (Rhodopsin-like receptors) | ECE1/PENK/CXCL3/AGT/ACKR3/ACKR1/ |
| | NPY1R/CXCL2 |
| Acyl chain remodelling of PC | PLAAT3 |
| Signaling by BMP | BMP2 |
| Glycolysis | ENO1/PFKM |
| Budding and maturation of HIV virion | UBAP1 |
| Peroxisomal lipid metabolism | PHYH |
| Tight junction interactions | PARD3 |
| VEGFR2 mediated vascular permeability | CTNNB1 |
| Negative regulation of FGFRS signaling | SPRY2 |
| Glycogen metabolismi | GBE1 |
| Nonsense-Mediated Decay (NMD) | CASC3/RPL22/RPS4Y1 |
| Nonsense Mediated Decay (NMD) enhanced by the Exon | CASC3/RPL22/RPS4Y1 |
| Junction Complex (EJC) | |
| Acyl chain remodelling of PE | PLAAT3 |
| EPHA-mediated growth cone collapse | YES1 |
| SUMOylation of intracellular receptors | NR3C2 |
| Myogenesis | CTNNB1 |
| MET activates PTK2 signaling | LAMA2 |
| Signaling by NOTCH1 | HES1/MYC |
| Signaling by FGFR2 | RBFOX2/SPRY2 |
| Regulation of RUNX2 expression and activity | PPARGC1A/BMP2 |
| NEP/NS2 interacts with the Cellular Export Machinery | RAN |
| Trafficking of AMPA receptors | TSPAN7 |
| Glutamate binding, activation of AMPA receptors and | TSPAN7 |
| synaptic plasticity | |
| MAPK targets/Nuclear events mediated by MAP kinases | FOS |
| Disassembly of the destruction complex and recruitment | CTNNB1 |
| of AXIN to the membrane | |
| Negative regulation of FGFR4 signaling | SPRY2 |
| Pyruvate metabolism | PDX4 |
| Cristae formation | HSPA9 |
| FCERI mediated MAPK activation | FOS |
| RHO GTPases activate IQGAPs | CTNNB1 |
| RNA Polymerase I Transcription Termination | CAVIN1 |
| Endosomal Sorting Complex Required For Transport | UBAP1 |
| (ESCRT) | |
| ECM proteoglycans | ITGA7/LAMA2 |
| Export of Viral Ribonucleoproteins from Nucleus | RAN |
| Nuclear import of Rev protein | RAN |
| Signaling by NOTCH2 | HES1 |
| Oncogene Induced Senescence | ETS2 |
| CD28 co-stimulation | YES1 |
| Adherens junctions interactions | CTNNB1 |
| Negative regulation of FGFR1 signaling | SPRY2 |
| Resolution of D-loop Structures through Holliday | XRCC2 |
| Junction Intermediates | |
| Cargo concentration in the ER | AREG |
| Biosynthesis of the N-glycan precursor (dolichol lipid- | GFPT2/UAP1 |
| linked oligosaccharide, LLO) and transfer to a nascent | |
| protein | |
| Rev-mediated nuclear export of HIV RNA | RAN |
| Synthesis of bile acids and bile salts | AKR1C1 |
| Metabolism of steroid hormones | AKR1B1 |
| Negative regulation of FGFR2 signaling | SPRY2 |
| Diseases of carbohydrate metabolism | GBE1 |
| Resolution of D-Loop Structures | XRCC2 |
| Amino acid synthesis and interconversion | GLUL |
| (transamination) | |
| Factors involved in megakaryocyte development and | HBB/PRKAR2B/ZFPM2/H3F3B |
| platelet production | |
| G alpha (12/13) signalling events | NET1/RHOB |
| GPVI-mediated activation cascade | RHOB |
| Glutathione conjugation | HPGD5 |
| Interactions of Rev with host cellular proteins | RAN |
| Inactivation, recovery and regulation of the | METAP2 |
| phototransduction cascade | |
| Signaling by high-kinase activity BRAF mutants | PEBP1 |
| Cell-Cell communication | PARD3/CTNNB1/FERMT2 |
| The phototransduction cascade | METAP2 |
| Apoptotic cleavage of cellular proteins | CTNNB1 |
| Gene and protein expression by JAK-STAT signaling after | HSPA9 |
| interleukin-12 stimulation | |
| Toll Like Receptor 10 (TLR10) Cascade | PELI1/FOS |
| Toll Like Receptor S (TLR5) Cascade | PELI1/FOS |
| MyD88 cascade initiated on plasma membrane | PELI1/FOS |
| Metabolism of polyamines | SAT1/NQO1 |
| Translesion synthesis by Y family DNA polymerases | REV3L |
| bypasses lesions on DNA template | |
| Association of TriC/CCT with target proteins during | NOP56 |
| biosynthesis | |
| Presynaptic phase of homologous DNA pairing and | XRCC2 |
| strand exchange | |
| GABA B receptor activation | ADCY2 |
| Activation of GABAB receptors | ADCY2 |
| Signaling by FGFR | RBFOX2/SPRY2 |
| Signaling by FGFR3 | SPRY2 |
| MAP2K and MAPX activation | PEBP1 |
| Platelet homeostasis | PDE2A/APOB |
| Regulation of mRNA stability by proteins that bind AU- | ZFP36L1/2FP36 |
| rich elements | |
| Peptide chain elongation | RPL22/RPS4Y1 |
| Viral mRNA Translation | RPL22/RP54Y1 |
| Diseases associated with glycosaminoglycan metabolism | SDC4 |
| Signaling by FGFR4 | SPRY2 |
| RNA Polymerase III Transcription | NFIB |
| RNA Polymerase III Abortive And Retractive Initiation | NFIB |
| RET signaling | IRS2 |
| MET promotes cell motility | LAMA2 |
| Beta defensins | DEFB1 |
| Glucagon-like Peptide-1 (GLP1) regulates insulin | PRKAR2B |
| secretion | |
| Homologous DNA Pairing and Strand Exchange | XRCC2 |
| Opioid Signalling | ADCY2/PRKAR2B |
| Late Phase of HIV Life Cycle | TAF7/UBAP1/RAN |
| EPH-Ephrin signaling | YES1/EFNB2 |
| Regulation of TP53 Activity through Phosphorylation | TAF7/NUAK1 |
| TRAF6 mediated induction of NFkB and MAP kinases | PELI1/FOS |
| upon TLR7/8 or 9 activation | |
| Interleukin-1 family signaling | PELI1/SMAD3/IL1R1 |
| Bile acid and bile salt metabolism | AKR1C1 |
| Neddylation | KLHL21/SPSB1/SOCS2/SOCS3/2BTB16 |
| Eukaryotic Translation Elongation | RPL22/BPS4Y1 |
| Toll Like Receptor 7/8 (TLR7/8) Cascade | PELI1/FOS |
| Selenocysteine synthesis | RPL22/RPS4Y1 |
| Eukaryotic Translation Termination | RPL22/RPS4Y1 |
| MyD88 dependent cascade initiated on endosome | PELI1/FOS |
| Cardiac conduction | RYR3/KCNK3/ATP1A2 |
| Signaling by NOTCH | ACTA2/SMADS/H3F3B/HES1/MYC |
| PI3K Cascade | IRS2 |
| Transport of vitamins, nucleosides, and related | APOD |
| molecules | |
| Recruitment of NuMA to mitotic centrosomes | NUMA1/PRKAR2B |
| Diseases of glycosylation | ADAMTS5/SDC4/ADAMTS1 |
| Mitochondrial biogenesis | HSPA9/PPARGC1A |
| MyD88:MAL(TIRAP) cascade initiated on plasma | PELI1/FOS |
| membrane | |
| Toll Like Receptor TLR6:TLR2 Cascade | PELI1/FOS |
| RNO GTPases activate PKNs | H3FB/RHOB |
| Nonsense Mediated Decay (NMD) independent of the | RPL22/APSAY1 |
| Exon Junction Complex (EJC) | |
| Influenza Life Cycle | RPL22/RAN/RPS4Y1 |
| Toll Like Receptor 9 (TLR9) Cascade | PELI1/FOS |
| Toll Like Receptor TLR1:TLA2 Cascade | PELI1/FOS |
| Toll Like Receptor 2 (TLR2) Cascade | PELI1/FOS |
| HIV Transcription Initiation | TAF7 |
| RNA Polymerase II HIV Promoter Escape | TAF7 |
| Signaling by moderate kinase activity BRAF mutants | PEBP1 |
| Paradoxical activation of RAF signaling by kinase inactive | PEBP1 |
| BRAF | |
| RNA Polymerase II Promoter Escape | TAF7 |
| RNA Polymerase II Transcription Pre-Initiation And | TAF7 |
| Promoter Opening | |
| RNA Polymerase II Transcription Initiation | TAF7 |
| RNA Polymerase II Transcription Initiation And Promoter | TAF7 |
| Clearance | |
| Interleukin-12 signaling | HSPA9 |
| Infectious disease | TAF7/UBAP1/HSP90AB1/RPL22/RAN/CTNNB1/HBEGF/RPS4Y1 |
| VEGFA-VEGFR2 Pathway | CTNNB1/VEGFA |
| IRS-mediated signalling | IRS2 |
| Interleukin-3, Interleukin-5 and GM-CSF signaling | YES1 |
| Signaling by Hedgehog | ADCY2/PRKAR2B/GAS1 |
| Retrograde transport at the Trans-Golgi-Network | RHOBTB3 |
| DNA Damage Bypass | REV3L |
| Signaling by NOTCH3 | HES1 |
| Formation of a pool of free 40S sabersits | RPL22/AP54Y1 |
| HIV Life Cycle | TAF7/UBAP1/RAN |
| Inositol phosphate metabolism | INPP5A |
| HDMs demethylate histones | KDM3D |
| Signaling by FGFR1 | SPRY2 |
| Interleukin-1 signaling | PELI1/IL1R1 |
| Neurotransmitter release cycle | MAOA |
| Regulation of ornithine decarboxylase (ODC) | NQO1 |
| Formation of the ternary complex, and subsequently, the | RPS4Y1 |
| 43S complex | |
| Influenza Infection | RPL22/RAN/RPS4Y1 |
| Toll-like Receptor Cascades | PELI1/APOB/FOS |
| Defensins | DEFB1 |
| IRS-related events triggered by IGF1R | IRS2 |
| Nuclear Receptor transcription pathway | NR3C2 |
| mRNA Splicing - Minor Pathway | SRSF7 |
| Transcriptional regulation by small RNAs | RAN/M3F3B |
| IGF1R signaling cascade | IRS2 |
| Signsling by VEGF | CTNNB1/VEGFA |
| NoRC negatively regulates rRNA expression | SAP18/H3F3B |
| Insulin receptor signalling cascade | IRS2 |
| Complex I biogenesis | NDUFAF4 |
| Pyruvate metabolism and Citric Acid (TCA) cycle | PDK4 |
| Negative epigenetic regulation of rRNA expression | SAP18/H3F3B |
| Phospholipid metabolism | GPD1L/PNPLA2/PLAAT3/GPD1 |
| Transcriptional activation of mitochondrial biogenesis | PPARGC1A |
| GABA receptor activation | ADCY2 |
| Platelet activation, signaling and aggregation | CDC37L1/VEGFA/TIMP3/RHOB/CFD |
| L13a-mediated translational silencing of Ceruloplasmin | RPL22/RPS4Y1 |
| expression | |
| Protein localization | PEX5/HSPA9/PHYH |
| SRP-dependent cotranslational protein targeting to | RPL22/RPS4Y1 |
| membrane | |
| O-linked glycosylation | ADAMTS5/ADAMTS1 |
| GTP hydrolysis and joining of the 60S ribosomal subunit | RPL22/RPS4Y1 |
| RNA Polymerase I Transcription | CAVIN1/H3F3B |
| tRNA processing in the nucleus | RAN |
| Hedgehog ‘off’ state | ADCY2/PRKAR2B |
| Signaling by PDGF | THBS4 |
| Translation initiation complex formation | RPS4Y1 |
| Ribosomal scanning and start codon recognition | RPS4Y1 |
| G alpha (q) signalling events | GRX5/AGT/RG516/HBEGF |
| NRAGE signals death through JNK | NET1 |
| Activation of the mRNA upon binding of the cap-binding | RPS4Y1 |
| complex and eIF's, and subsequent binding to 43S | |
| E3 ubiquitin ligases ubiquitinate target proteins | PEX5 |
| Signaling by RAS mutants | PEBP1 |
| Transmission across Chemical Synapses | ADCY2/GLUL/PRKAR2B/TSPAN7/MAOA |
| Regulation of expression of SLITs and ROBOS | CASC3/RPL22/RPS4Y1 |
| Meiosis | SUN1/H3F3B |
| Selenoamino acid metabolism | RPL22/RPS4Y1 |
| rRNA modification in the nucleus and cytosol | NOP56 |
| Transcriptional Regulation by MECP2 | FKBPS |
| Eukaryotic Translation Initiation | RPL22/RPS4Y1 |
| Cap-dependent Translation Initiation | RPL22/RPS4Y1 |
| Cytosolic sensors of pathogen-associated DNA | CTNNB1 |
| RNA Polymerase I Promoter Opening | H3F3B |
| Mitochondrial protein import | HSPA9 |
| Collagen degradation | MMP14 |
| MAP kinase activation | FOS |
| DNA methylation | H3F3B |
| TP53 Regulates Transcription of DNA Repair Genes | FOS |
| Oxidative Stress Induced Senescence | H3F3B/FOS |
| Collagen biosynthesis and modifying enzymes | PCOLCE2 |
| Activated PKN1 stimulates transcription of AR (androgen | H3F3B |
| receptor) regulated genes KLX2 and KLK3 | |
| HDR through Homologous Recombination (HRR) | XRCC2 |
| Signaling by BRAF and RAF fusions | PEBP1 |
| COPII-mediated vesicle transport | AREG |
| SIRT1 negatively regulates rRNA expression | H3F3B |
| SUMO E3 ligases SUMOylate target proteins | PPARGC1A/NR3C2/NRIP1 |
| Toll Like Receptor 4 (TLR4) Cascade | PELI1/FOS |
| Loss of Nlp from mitotic centrosomes | PRKAR2B |
| Loss of proteins required for interphase microtubule | PRKAR2B |
| organization from the centrosome | |
| Costimulation by the CD2B family | YES1 |
| Major pathway of rRNA processing in the nucleokis and | NOP56/RPL22/APS4Y1 |
| cytosol | |
| Ion channel transport | CUTC/RYR3/ATP1A2 |
| ISG15 antiviral mechanism | FLNB |
| Interleukin-17 signaling | FOS |
| SUMOylation | PPARGC1A/NR3C2/NRIP1 |
| Transcription of the HIV genome | TAF7 |
| PRC2 methylates histones and DNA | H3F3B |
| AURKA Activation by TPX2 | PRKAR2B |
| Influenza Viral RNA Transcription and Replication | RPL22/RPS4Y1 |
| Condensation of Prophase Chromosomes | H3F3B |
| Gene Silencing by RNA | RAN/M3F3B |
| Cellular response to hypoxia | VEGFA |
| Cell death signaling via NRAGE, NRIF and NADE | NET1 |
| ERCC6 (CSB) and ENMT2 (G9s) positively regulate rRNA | H3F3B |
| expression | |
| rRNA processing in the nucleus and cytosol | NOP56/RPL22/RP54Y1 |
| The role of GTSE1 in G2/M progression after G2 | HSP90AB1 |
| checkpoint | |
| G2/M Transition | HSP90AB1/PHLDA1/PRKAR2B |
| SUC-mediated transmembrane transport | SLC47A1/SLC16A7/CP/APOD |
| PTEN Regulation | SNAI1/EGR1 |
| Signaling by NTRK1 (TRKA) | IRS2 |
| Regulation of insulin secretion | PAKAR2B |
| Signaling by insulin receptor | IRS2 |
| Mitotic G2-G2/M phases | HSP90AB1/PHLDA1/PRKAR2B |
| Meiotic synapsis | SUN1 |
| Signaling by MET | LAMA2 |
| Protein ubiquitination | PEXS |
| Interferon Signaling | FLNB/SOCS3/EGR1 |
| Mitotic Prophase | NUMA1/H3F3B |
| Antiviral mechanisms by IFN-stimulated genes | FLNB |
| DNA Damage/Telomere Stress Induced Senescence | H1F0 |
| Antigen processing: Ubiquitination & Proteasome | KLNL21/SPSB1/CBLB/SOCS3/ZBTB16 |
| degradation | |
| Reproduction | SUN1/H3F3B |
| Post NMDA receptor activation events | PRKAR2B |
| Recruitment of mitotic centrosome proteins and | PRKAR2B |
| complexes | |
| Centrosome maturation | PRKAR2B |
| Transcriptional Regulation by TP53 | TAF7/NUAK1/RGCC/BTG2/GADD45A/FOS |
| Oncogenic MAPK signaling | PEBP1 |
| Cyclin E associated events dering G1/S transition | MYC |
| rRNA processing | NOP56/RPL22/RP54Y1 |
| Neurotransmitter receptors and postsynaptic signal | ADCY2/PAKAR2B/TSPAN2 |
| transmission | |
| RNA Polymerase II Pre-transcription Events | TAF7 |
| Epigenetic regulation of gene expression | SAP18/H3F3B |
| Integrin cell surface interactions | ITGA7 |
| Hedgehog ‘on’ state | GAS1 |
| Cyclin A:Cdk2-associated events at S phase entry | MYC |
| Meiotic recombination | H3F3B |
| Regulation of PLK1 Activity at G2/M Transition | PRKAR2B |
| Collagen formation | PCOLCE2 |
| B-WICH complex positively regulates rRNA expression | H3F3B |
| RNA Polymerase I Promoter Escape | H3F3B |
| Macroautophagy | GABARAPL1 |
| PCP/CE pathway | WNT11 |
| Interferon gamma signaling | SOC53 |
| Signaling by ROBO receptors | CASC3/RPL22/RPS4Y1 |
| Post-translational modification: Synthesis of GPI- | RECK |
| anchored proteins | |
| Pre-NOTCH Transcription and Translation | H3F3B |
| Activation of NMDA receptors and postsynaptic events | PRICAR2B |
| Regulation of TP53 Activity | TAF7/NUAK1 |
| Toll Like Receptor 3 (TLR3) Cascade | FOS |
| HDACs deacetylate histones | SAP18 |
| Chaperonin-mediated protein folding | NOP56 |
| p75 NTR receptor mediated signalling | NET1 |
| Antimicrobial peptides | DEFB1 |
| RUNX1 regulates genes involved in megakaryocyte | H3F3B |
| differentiation and platelet function | |
| SLC transporter disorders | CP |
| Anchoring of the basal body to the plasma membrane | PRKAR2B |
| Potassium Channels | KCNK3 |
| MyD88-independent TLR4 cascade | FOS |
| Signaling by NTRKs | IRS2 |
| TRIF(TICAM1)-mediated TLR4 signaling | FOS |
| Respiratory electron transport | NDUFAF4 |
| Protein folding | NOP56 |
| Signaling by Rho GTPases | NET1/TRIP10/ARHGAP29/KTN1/CTNNB1/H3F38/RHOB |
| UCH proteinases | TGFBR2 |
| HIV Infection | TAF7/UBAP1/RAN |
| ABC-family proteins mediated transport | ABCA8 |
| The citric acid (TCA) cycle and respiratory electron | NDUFAF4/PDK4 |
| transport | |
| Positive epigenetic regulation of rRNA expression | H3F3B |
| Apoptosis | H1F0/CTNNB1 |
| tRNA processing | RAN |
| Neuronal System | ADCY2/GLUL/PRKAR2B/KCNK3/TSPAN7/MAOA |
| Programmed Cell Death | H1F0/CTNNB1 |
| Pre-NOTCH Expression and Processing | H3F3B |
| Stimuli-sensing channels | RYR3 |
| Amyloid fiber formation | H3F3B |
| RNA Polymerase I Promoter Clearance | H3F3B |
| Class I MHC mediated antigen processing & presentation | KLHL21/SPSB1/CBLB/SOCS3/ZBTB16 |
| Activation of anterior HOX genes in hindbrain | H3F3B |
| development during early embryogenesis | |
| Activation of HOX genes during differentiation | H3F3B |
| Respiratory electron transport, ATP synthesis by | NDUFAF4 |
| chemiosmotic coupling, and heat production by | |
| uncoupling proteins. | |
| RHO GTPase Effectors | KTN1/CTNNB1/H3F3B/RHOB |
| Mitotic Prometaphase | NUMA1/PRKAR2B |
| Nost Interactions of HIV factors | RAN |
| RUNX1 regulates transcription of genes involved in | H3F3B |
| differentiation of HSCs | |
| G1/S Transition | MYC |
| HDR through Homologous Recombination (HRR) or | XRCC2 |
| Single Strand Annealing (SSA) | |
| Fc epsilon receptor (FCERI) signaling | FOS |
| Homology Directed Repair | XRCC2 |
| RHO GTPases Activate Formins | RHOB |
| Death Receptor Signalling | NET1 |
| Ub-specific processing proteases | SMAD3/MYC |
| Organelle biogenesis and maintenance | HSPA9/PPARGC1A/PRKAR2B |
| Mitotic G1-G1/S phases | MYC |
| Deubiquitination | SMAD3/TGFBR2/MYC |
| ER to Golgi Anterograde Transport | AREG |
| Asparagine N-linked glycosylation | GFPT2/AREG/UAP1 |
| Transcriptional regulation by RUNX1 | H3F3B/SOCS3 |
| S Phase | MYC |
| DNA Double-Strand Break Repair | XRCC2 |
| Disorders of transmembrane transporters | CP |
| Transport to the Golgi and subsequent modification | AREG |
| Neutrophil degranulation | HSP90AB1/FGL2/PRDX6/HBB/CFD |
| Chromatin modifying enzymes | SAP18/KDM5D |
| Chromatin organization | SAP18/KDM5D |
| Cilium Assembly | PRKAR2B |
| Intra-Golgi and retrograde Golgi-to-ER traffic | RHOBTB3 |
| Translation | RPL22/RPS4Y1 |
| M Phase | NUMA1/PRKAR2B/H3F3B |
| DNA Repair | XRCC2/REV3L |

TABLE 4
Significant Pathways - Blood

| Description | geneID |
|---|---|
| Interleukin-2 signaling | JAK1/IL2RB |
| Interleukin-15 signaling | JAK1/IL2RB |
| Signaling by Interleukins | CCL5/S1PR1/IL7R/JAK1/IL2RB/MYC |
| Interleukin receptor SHC signaling | JAK1/IL2RB |
| Interleukin-4 and Interleukin-13 signaling | S1PR1/JAK1/MYC |
| Uptake and actions of bacterial toxins | HSP90AB1/CD9 |
| Interleukin-7 signaling | IL7R/JAK1 |
| Immunoregulatory interactions between a | CD247/SIGLEC10/CD8A |
| Lymphoid and a non-Lymphoid cell | |
| Interleukin-2 family signaling | JAK1/IL2RB |
| Interleukin-10 signaling | CCL5/JAK1 |
| Interleukin-3, Interleukin-5 and GM-CSF signaling | JAK1/IL2RB |
| HSP90 chaperone cycle for steroid hormone | HSP90AB1/TUBB2A |
| receptors (SHR) | |
| mRNA 3′-end processing | ALYREF/SRRM1 |
| mRNA Splicing - Major Pathway | ALYREF/SRRM1/HNRNPL |
| Regulation of actin dynamics for phagocytic cup | CD247/HSP90AB1 |
| formation | |
| mRNA Splicing | ALYREF/SRRM1/HNRNPL |
| RNA Polymerase II Transcription Termination | ALYREF/SRRM1 |
| Infectious disease | CD247/HSP90AB1/CD9/RPS4Y1 |
| Transport of Mature mRNA derived from an Intron- | ALYREF/SRRM1 |
| Containing Transcript | |
| The role of GTSE1 in G2/M progression after G2 | HSP90AB1/TUBB2A |
| checkpoint | |
| Transport of Mature Transcript to Cytoplasm | ALYREF/SRRM1 |
| Fcgamma receptor (FCGR) dependent phagocytosis | CD247/HSP90AB1 |
| Processing of Capped Intron-Containing Pre-mRNA | ALYREF/SRRM1/HNRNPL |
| Signaling by TGF-beta family members | NOG/MYC |
| MAPK3 (ERK1) activation | JAK1 |
| Regulation of commissural axon pathfinding by SLIT | NELL2 |
| and ROBO | |
| Interleukin-21 signaling | JAK1 |
| Interleukin-6 signaling | JAK1 |
| Interleukin-27 signaling | JAK1 |
| FCGR activation | CD247 |
| HSF1 activation | HSP90AB1 |
| Interleukin-35 Signalling | JAK1 |
| MAPK family signaling cascades | JAK1/IL2RB/MYC |
| Attenuation phase | HSP90AB1 |
| DCC mediated attractive signaling | ABLIM1 |
| Lysosphingolipid and LPA receptors | S1PR1 |
| Regulation of IFNG signaling | JAK1 |
| The NLRP3 inflammasome | HSP90AB1 |
| Sema3A PAK dependent Axon repulsion | HSP90AB1 |
| IL-6-type cytokine receptor ligand interactions | JAK1 |
| Microtubule-dependent trafficking of connexons | TUBB2A |
| from Golgi to the plasma membrane | |
| Transcription of E2F targets under negative control | MYC |
| by DREAM complex | |
| Transport of connexons to the plasma membrane | TUBB2A |
| Translocation of ZAP-70 to Immunological synapse | CD247 |
| Inflammasomes | HSP90AB1 |
| Estrogen-dependent gene expression | HSP90AB1/MYC |
| Phosphorylation of CD3 and TCR zeta chains | CD247 |
| Post-chaperonin tubulin folding pathway | TUBB2A |
| RAF-independent MAPK1/3 activation | JAK1 |
| PD-1 signaling | CD247 |
| HSF1-dependent transactivation | HSP90AB1 |
| Interleukin-6 family signaling | JAK1 |
| Role of phospholipids in phagocytosis | CD247 |
| Formation of tubulin folding intermediates by | TUBB2A |
| CCT/TriC | |
| Interleukin-20 family signaling | JAK1 |
| Fertilization | CD9 |
| Other interleukin signaling | JAK1 |
| Regulation of IFNA signaling | JAK1 |
| G0 and Early G1 | MYC |
| The role of Nef in HIV-1 replication and disease | CD247 |
| pathogenesis | |
| Signaling by BMP | NOG |
| Prefoldin mediated transfer of substrate to | TUBB2A |
| CCT/TriC | |
| Activation of AMPK downstream of NMDARs | TUBB2A |
| Surfactant metabolism | ADA2 |
| SMAD2/SMAD3:SMAD4 heterotrimer regulates | MYC |
| transcription | |
| Cooperation of Prefoldin and TriC/CCT in actin and | TUBB2A |
| tubulin folding | |
| RHO GTPases activate IQGAPs | TUBB2A |
| Cellular responses to stress | ETS1/HSP90AB1/TUBB2A |
| G2/M Transition | HSP90AB1/TUBB2A |
| Generation of second messenger molecules | CD247 |
| Oncogene Induced Senescence | ETS1 |
| Mitotic G2-G2/M phases | HSP90AB1/TUBB2A |
| Transport of the SLBP Independent Mature mRNA | ALYREF |
| Transport of the SLBP Dependant Mature mRNA | ALYREF |
| Gap junction assembly | TUBB2A |
| Transcriptional regulation by the AP-2 (TFAP2) | MYC |
| family of transcription factors | |
| Carboxyterminal post-translational modifications | TUBB2A |
| of tubulin | |
| Signaling by ROBO receptors | NELL2/RPS4Y1 |
| Transport of Mature mRNA Derived from an | ALYREF |
| Intronless Transcript | |
| Assembly and cell surface presentation of NMDA | TUBB2A |
| receptors | |
| ESR-mediated signaling | HSP90AB1/MYC |
| Transport of Mature mRNAs Derived from | ALYREF |
| Intronless Transcripts | |
| Transcriptional activity of SMAD2/SMAD3:SMAD4 | MYC |
| heterotrimer | |
| Glycosphingolipid metabolism | ESYT1 |
| Gap junction trafficking | TUBB2A |
| NOTCH1 Intracellular Domain Regulates | MYC |
| Transcription | |
| Recycling pathway of L1 | TUBB2A |
| Interleukin-12 signaling | JAK1 |
| Chemokine receptors bind chemokines | CCL5 |
| Gap junction trafficking and regulation | TUBB2A |
| Netrin-1 signaling | ABLIM1 |
| RAF/MAP kinase cascade | JAK1/IL2RB |
| COPI-independent Golgi-to-ER retrograde traffic | TUBB2A |
| Formation of the ternary complex, and | RPS4Y1 |
| subsequently, the 435 complex | |
| MAPK1/MAPK3 signaling | JAK1/IL2RB |
| Intraflagellar transport | TUBB2A |
| Nucleotide-binding domain, leucine rich repeat | HSP90AB1 |
| containing receptor (NLR) signaling pathways | |
| Signaling by Nuclear Receptors | HSP90AB1/MYC |
| Interleukin-12 family signaling | JAK1 |
| Signaling by NOTCH1 PEST Domain Mutants in | MYC |
| Cancer | |
| Signaling by NOTCH1 in Cancer | MYC |
| Constitutive Signaling by NOTCH1 PEST Domain | MYC |
| Mutants | |
| Signaling by NOTCH1 HD + PEST Domain Mutants in | MYC |
| Cancer | |
| Constitutive Signaling by NOTCH1 HD + PEST Domain | MYC |
| Mutants | |
| Translation initiation complex formation | RPS4Y1 |
| Ribosomal scanning and start codon recognition | RPS4Y1 |
| Activation of the mRNA upon binding of the cap- | RPS4Y1 |
| binding complex and eIFs, and subsequent binding | |
| to 435 | |
| Kinesins | TUBB2A |
| Semaphorin interactions | HSP90AB1 |
| Interferon alpha/beta signaling | JAK1 |
| Costimulation by the CD28 family | CD247 |
| Asparagine N-linked glycosylation | TUBB2A/STT3A |
| ISG1S antiviral mechanism | JAK1 |
| Translocation of SLC2A4 (GLUT4) to the plasma | TUBB2A |
| membrane | |
| Signaling by TGF-beta Receptor Complex | MYC |
| Signaling by NOTCH1 | MYC |
| Class A/1 (Rhodopsin-like receptors) | CCL5/S1PR1 |
| Antiviral mechanism by IFN-stimulated genes | JAK1 |
| Post NMDA receptor activation events | TUBB2A |
| Cyclin E associated events during G1/S transition | MYC |
| Cyclin A:Cdk2-associated events at S phase entry | MYC |
| Peptide chain elongation | RPS4Y1 |
| Viral mRNA Translation | RPS4Y1 |
| Cellular response to heat stress | HSP90AB1 |
| Sphingolipid metabolism | ESYT1 |
| MAPK6/MAPK4 signaling | MYC |
| Formation of the beta-catenin:TCF transactivating | MYC |
| complex | |
| Interferon gamma signaling | JAK1 |
| Eukaryotic Translation Elongation | RPS4Y1 |
| Selenocysteine synthesis | RPS4Y1 |
| Activation of NMDA receptors and postsynaptic | TUBB2A |
| events | |
| Eukaryotic Translation Termination | RPS4Y1 |
| Recruitment of NuMA to mitotic centrosomes | TUBB2A |
| Chaperonin-mediated protein folding | TUBB2A |
| Nonsense Mediated Decay (NMD) independent of | RPS4Y1 |
| the Exon Junction Complex (EJC) | |
| Transcriptional regulation by RUNX3 | MYC |
| Downstream TCR signaling | CD247 |
| COPI-dependent Golgi-to-ER retrograde traffic | TUBB2A |
| Protein folding | TUBB2A |
| COPI-mediated anterograde transport | TUBB2A |
| Formation of a pool of free 40S subunits | RPS4Y1 |
| Phase I - Functionalization of compounds | HSP90AB1 |
| Cargo recognition for clathrin-mediated | IL7R |
| endocytosis | |
| Stimuli-sensing channels | WNK1 |
| L13a-mediated translational silencing of | RPS4Y1 |
| Ceruloplasmin expression | |
| SRP-dependent cotranslational protein targeting to | RPS4Y1 |
| membrane | |
| GTP hydrolysis and joining of the 60S ribosomal | RPS4Y1 |
| subunit | |
| Hedgehog ‘off’ state | TUBB2A |
| Nonsense-Mediated Decay (NMD) | RPS4Y1 |
| Nonsense Mediated Decay (NMD) enhanced by the | RPS4Y1 |
| Exon Junction Complex (EJC) | |
| G alpha (i) signalling events | CCL5/S1PR1 |
| Selenoamino acid metabolism | RPS4Y1 |
| TCR signaling | CD247 |
| L1CAM interactions | TUBB2A |
| Eukaryotic Translation Initiation | RPS4Y1 |
| Cap-dependent Translation Initiation | RPS4Y1 |
| MHC class II antigen presentation | TUBB2A |
| Resolution of Sister Chromatid Cohesion | TUBB2A |
| Platelet degranulation | CD9 |
| Host Interactions of HIV factors | CD247 |
| G1/S Transition | MYC |
| Golgi-to-ER retrograde transport | TUBB2A |
| Influenza Viral RNA Transcription and Replication | RPS4Y1 |
| Response to elevated platelet cytosolic Ca2+ | CD9 |
| RHO GTPases Activate Formins | TUBB2A |
| GPCR ligand binding | CCL5/S1PR1 |
| Reproduction | CD9 |
| Influenza Life Cycle | RPS4Y1 |
| Clathrin-mediated endocytosis | IL7R |
| Mitotic G1-G1/S phases | MYC |
| Signaling by Hedgehog | TUBB2A |
| ER to Golgi Anterograde Transport | TUBB2A |
| Neutrophil degranulation | ADA2/HSP90AB1 |
| Influenza infection | RPS4Y1 |
| S Phase | MYC |
| Factors involved in megakaryocyte development | TUBB2A |
| and platelet production | |
| Regulation of expression of SLITs and ROBOs | RPS4Y1 |
| Major pathway of rRNA processing in the nucleolus | RPS4Y1 |
| and cytosol | |
| Transport to the Golgi and subsequent | TUBB2A |
| modification | |
| Ion channel transport | WNK1 |
| Separation of Sister Chromatids | TUBB2A |
| Peptide ligand-binding receptors | CCL5 |
| Cellular Senescence | ETS1 |
| rRNA processing in the nucleus and cytosol | RPS4Y1 |
| Interferon Signaling | JAK1 |
| Mitotic Prometaphase | TUBB2A |
| Cilium Assembly | TUBB2A |
| Mitotic Anaphase | TUBB2A |
| Mitotic Metaphase and Anaphase | TUBB2A |
| Intra-Golgi and retrograde Golgi-to-ER traffic | TUBB2A |
| rRNA processing | RPS4Y1 |
| Neurotransmitter receptors and postsynaptic signal | TUBB2A |
| transmission | |
| Ub-specific processing proteases | MYC |
| Biological oxidations | HSP90AB1 |
| HIV Infection | CD247 |
| TCF dependent signaling in response to WNT | MYC |
| Signaling by NOTCH | MYC |
| Platelet activation, signaling and aggregation | CD9 |
| Transmission across Chemical Synapses | TUBB2A |
| Translation | RPS4Y1 |
| Organelle biogenesis and maintenance | TUBB2A |
| Deubiquitination | MYC |
| RHO GTPase Effectors | TUBB2A |
| Signaling by WNT | MYC |
| Metabolism of amino acids and derivatives | RPS4Y1 |
| Diseases of signal transduction | MYC |
| M Phase | TUBB2A |
| Neuronal System | TUBB2A |
| Signaling by Rho GTPases | TUBB2A |

When evaluating the overlap between differentially expressed genes in synovium and blood, there were 28 genes commonly up-regulated: TNFAIP6, S100A8, MMP9, S100A9, IFI27, EVI2A, NMI, BCL2A1, TNFSF10, LY96, SAMSN1, GPR65, DDX60, ISG15, MX1, OAS1, IF144, ENTPD1, IFIT3, CSTA, CLIC1, IFIT1, DOCK4, NATI, FAS, C1GALT1C1, CD58, COMMD8; and 4 down-regulated genes: SIPR1, TUBB2A, ABLIM1, MYC (FIG. 2C). However, the overlap of down-regulated genes did not meet statistical significance: p=9e-9 for up-regulated genes and p=0.28 for down-regulated genes (FIG. 2D). The common differentially expressed (DE) genes formed more distinct clusters of RA and control samples for both synovium (FIG. 2E, FIG. 2F) and blood (FIG. 2G, FIG. 2H) than all DE genes for these tissues (FIG. 7A, FIG. 7B, FIG. 8A, and FIG. 8B). The Gene Ontology biological processes of these common up-regulated genes included innate immune and defense response, neutrophil degranulation and type I interferon signaling pathways, whereas down-regulated genes are associated with PDGFR-beta signaling and Interleukin-4 and 13 signaling pathways. Interestingly, the genes involved in interferon pathways showed the negative correlation between tissues (r=−0.78, 95% CI (−0.97, −0.07), p=0.04), whereas the genes involved in cell activation and neutrophil degranulation pathways correlated positively: r=0.7 (p=0.03) and rho=0.8 (p=0.1), respectively.

ii. Cell-Type Deconvolution Analysis Identifies a Reverse Signalin Blood and Synovium

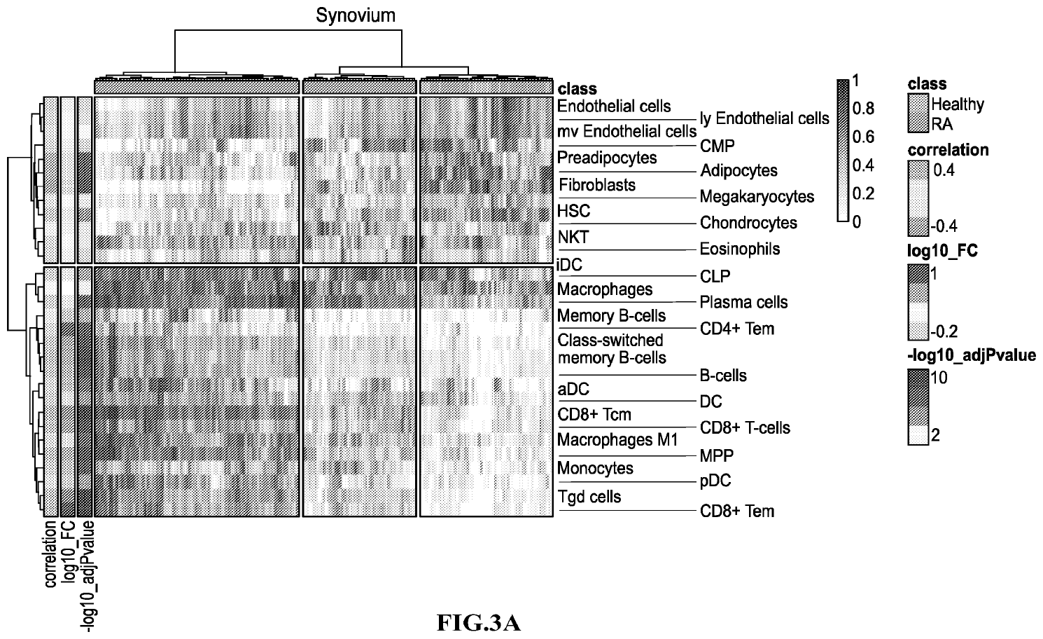

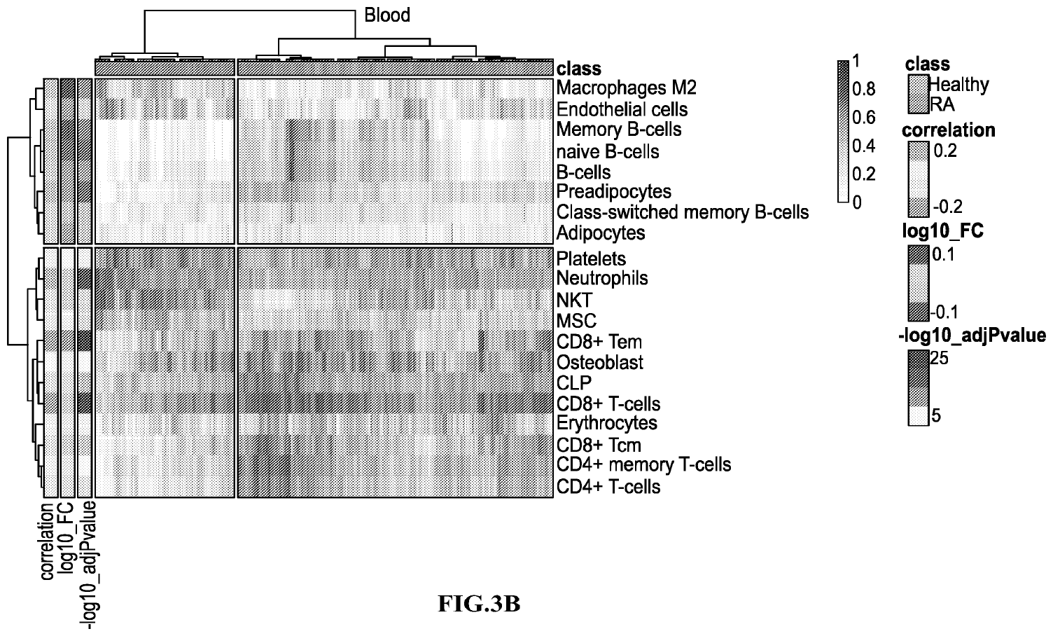

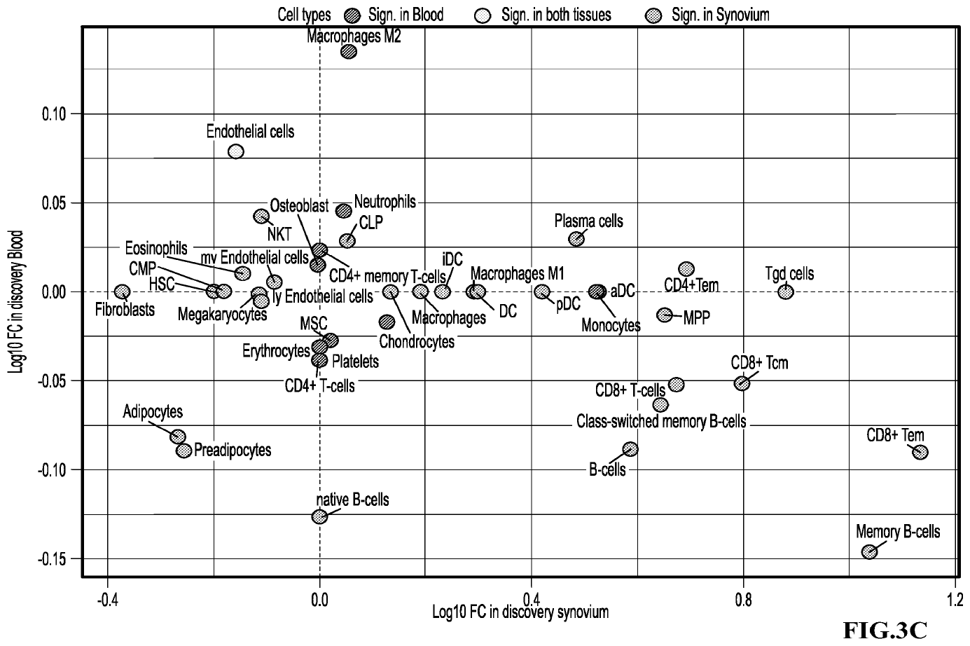

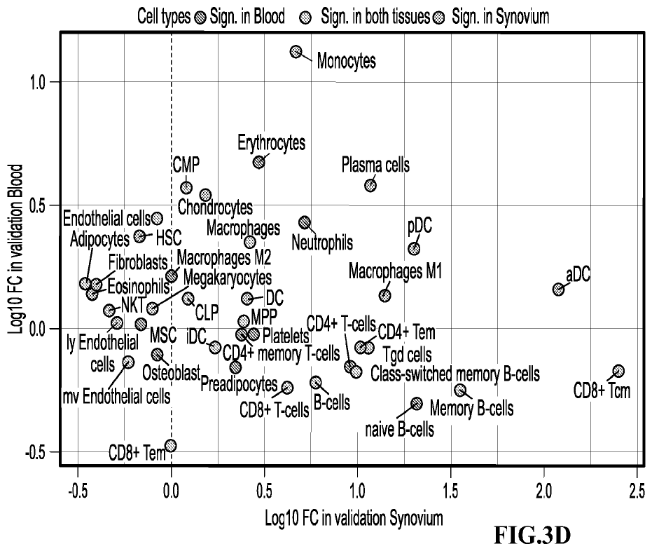

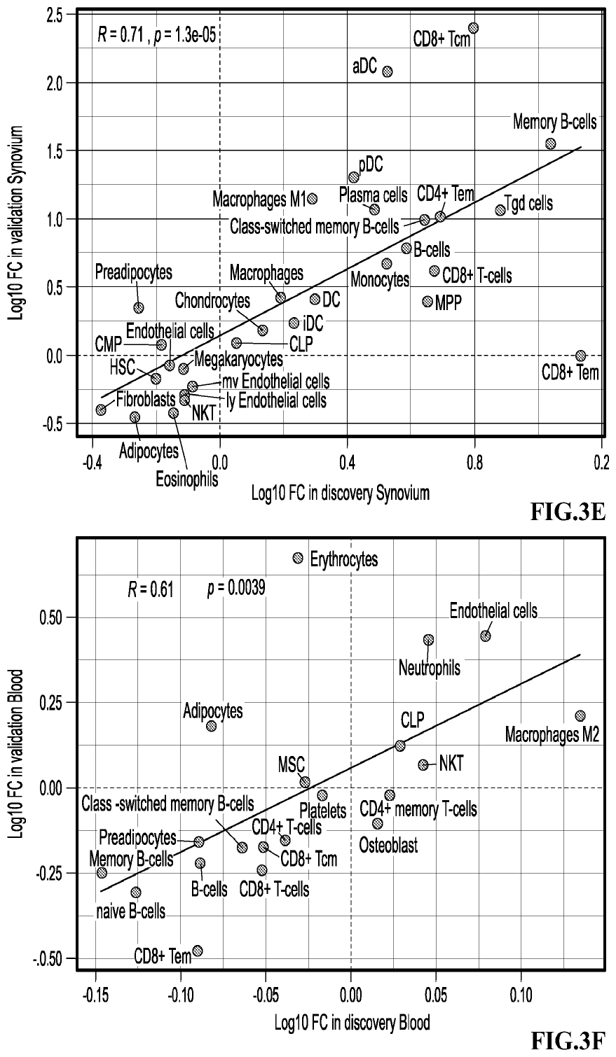

The cell type enrichment analysis with xCell in synovium revealed the enrichment of immune cell types, including, CD4+ and CD8+ T-cells, B-cells, macrophages and dendritic cells in RA samples (FIG. 3A). However, opposite results were seen in whole blood samples with enrichment of T- and B-cells in healthy controls (FIG. 3B). Concordance in activation of innate immune cells and opposition in activation of lymphocytes in tissues from discovery cohorts (FIG. 3C) were confirmed with validation datasets (FIG. 3D). The significant cell types in synovium and blood showed high correlations in validation data: r=0.71 (p=1.3e-5) for synovium (FIG. 3E) and r=0.61 (p=0.004) in blood (FIG. 3F).

iii. Machine Learning Feature Selection Strategy to Identify Robust Cross-Tissue Biomarkers of RA

Aiming to determine a more robust list of putative biomarkers that are strongly associated with RA in both synovium and whole blood tissues and have higher predictive power, we applied a feature selection procedure leveraging the gene expression data from both tissues. In the pipeline, only 10,071 genes that were common between synovium and whole blood data were used. At each iteration, only genes found significantly dysregulated in both tissues following the condition of co-directionality were kept (p=6.3e-10). As a result of these filtering steps, 65±1 up-regulated and 71±1 down-regulated were selected from each iteration (See Methods).

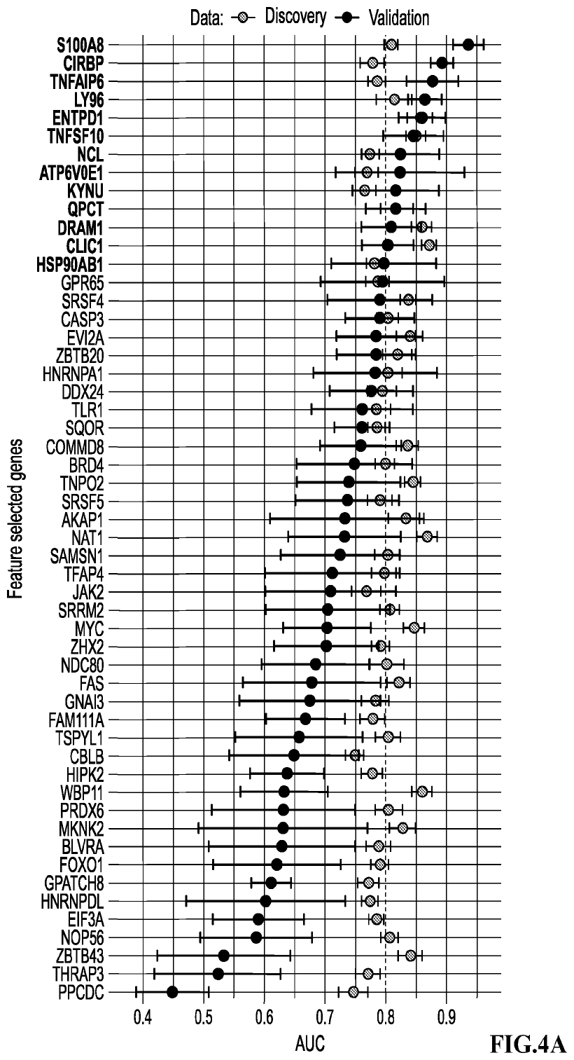

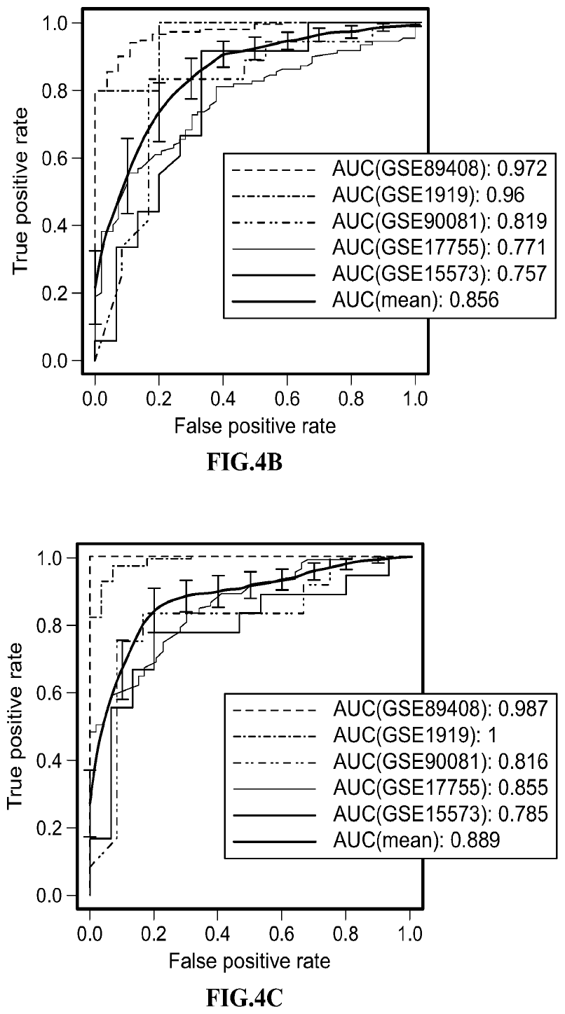

From 100 iterations, any gene significantly dysregulated in all the iterations was selected, resulting in a set of 53 genes: 25 up-regulated and 28 down-regulated (Table 5). A summary of the average AUC performance from the 100 iterations for each gene are shown in FIG. 4A and Table 7. The AUC for selected genes in synovium tissue varied with mean 0.853±0.005 for training and 0.866±0.006 for testing sets, whereas for the blood tissue the mean AUC was 0.744±0.006 for both training and testing sets.

TABLE 5
53 Feature Selected Genes

| | | Synovium | | | | Blood | | | |
|---|---|---|---|---|---|---|---|---|---|
| | | FC (BH adj. | | corr (BH adj. | | FC (BH adj. | | corr (BH adj. | |
| Gene | Description | p-value) | | p-value) | | p-value) | | p-value) | |
| TNFAIP6 | TNF Alpha Induced Protein 6 | 2.46 | (4E−06) | 0.39 | (7E−11) | 1.36 | (8E−16) | 0.39 | (3E−67) |
| S100A8 | S100 Calcium Binding Protein A8 | 2.28 | (7E−05) | 0.34 | (1E−08) | 1.46 | (7E−32) | 0.48 | (9E−108) |
| MMP9 | Matrix Metallopeptidase 9 | 2.13 | (2E−04) | 0.32 | (7E−08) | 1.27 | (1E−05) | 0.25 | (4E−27) |
| S100A9 | S100 Calcium Binding Protein A9 | 2.09 | (1E−04) | 0.34 | (2E−08) | 1.23 | (3E−22) | 0.41 | (4E−77) |
| IFI27 | Interferon Alpha Inducible Protein 27 | 1.87 | (7E−08) | 0.44 | (5E−14) | 1.3 | (4E−03) | 0.17 | (1E−13) |
| EVI2A | Ecotropic Viral Integration Site 2A | 1.66 | (2E−06) | 0.45 | (1E−14) | 1.48 | (4E−23) | 0.41 | (3E−76) |
| NMI | N-Myc And STAT Interactor | 1.66 | (4E−10) | 0.52 | (7E−20) | 1.22 | (1E−16) | 0.37 | (3E−61) |
| BCL2A1 | BCL2 Related Protein A1 | 1.62 | (1E−03) | 0.3 | (7E−07) | 1.46 | (1E−27) | 0.47 | (1E−101) |
| TNFSF10 | TNF Superfamily Member 10 | 1.55 | (1E−09) | 0.52 | (3E−19) | 1.27 | (1E−23) | 0.44 | (4E−88) |
| LY96 | Lymphocyte Antigen 96 | 1.54 | (1E−09) | 0.51 | (2E−18) | 1.22 | (7E−11) | 0.28 | (2E−35) |
| SAMSN1 | SAM Domain, SH3 Domain And | 1.52 | (1E−05) | 0.42 | (1E−12) | 1.23 | (3E−13) | 0.32 | (2E−46) |
| | Nuclear Localization Signals 1 | | | | | | | | |
| GPR65 | G Protein-Coupled Receptor 65 | 1.5 | (2E−05) | 0.39 | (5E−11) | 1.21 | (2E−13) | 0.31 | (4E−41) |
| DDX60 | DExD/H-Box Helicase 60 | 1.4 | (2E−08) | 0.48 | (8E−17) | 1.24 | (3E−06) | 0.26 | (2E−30) |
| ISG15 | ISG15 Ubiquitin Like Mixiifier | 1.37 | (4E−03) | 0.25 | (3E−05) | 1.43 | (1E−07) | 0.3 | (6E−39) |
| MX1 | MX Dynamin Like GTPase 1 | 1.37 | (3E−03) | 0.27 | (8E−06) | 1.21 | (6E−03) | 0.19 | (3E−16) |
| OAS1 | 2′-5′-Oligoadenylate Synthetase 1 | 1.36 | (4E−04) | 0.31 | (2E−07) | 1.31 | (4E−07) | 0.29 | (4E−36) |
| IFI44 | Interferon Induced Protein 44 | 1.35 | (6E−04) | 0.31 | (2E−07) | 1.42 | (7E−07) | 0.26 | (1E−30) |
| ENTPD1 | Ectonucleoside Triphosphate | 1.33 | (1E−08) | 0.52 | (2E−19) | 1.21 | (2E−16) | 0.4 | (5E−71) |
| | Diphosphohydrolase 1 | | | | | | | | |
| IFIT3 | Interferon Induced Protein With | 1.33 | (5E−03) | 0.24 | (4E−05) | 1.39 | (1E−09) | 0.32 | (8E−46) |
| | Tetratricopeptide Repeats 3 | | | | | | | | |
| CSTA | Cystatin A | 1.32 | (8E−04) | 0.3 | (7E−07) | 1.36 | (4E−22) | 0.42 | (5E−79) |
| CLIC1 | Chloride Intracellular Channel 1 | 1.32 | (5E−08) | 0.47 | (7E−16) | 1.2 | (5E−27) | 0.47 | (4E−103) |
| IFIT1 | Interferon Induced Protein With | 1.24 | (3E−02) | 0.2 | (7E−04) | 1.58 | (3E−10) | 0.32 | (1E−45) |
| | Tetratricopeptide Repeats 1 | | | | | | | | |
| DOCK4 | Dedicator Of Cytokinesis 4 | 1.23 | (1E−03) | 0.32 | (1E−07) | 1.22 | (2E−10) | 0.32 | (5E−44) |
| NAT1 | N-Acetyltransferase 1 | 1.23 | (6E−07) | 0.47 | (4E−16) | 1.2 | (1E−23) | 0.44 | (2E−88) |
| FAS | Fas Cell Surface Death Receptor | 1.22 | (9E−05) | 0.39 | (6E−11) | 1.23 | (1E−18) | 0.4 | (1E−72) |
| C1GALT1C1 | C1GALT1 Specific Chaperons 1 | 1.21 | (2E−04) | 0.33 | (4E−08) | 1.26 | (7E−34) | 0.51 | (7E−123) |
| CD58 | CD58 Molecule | 1.21 | (4E−03) | 0.28 | (2E−06) | 1.25 | (1E−26) | 0.44 | (4E−89) |
| COMMD8 | COMM Domain Containing 8 | 1.21 | (2E−04) | 0.37 | (6E−10) | 1.29 | (2E−21) | 0.39 | (3E−69) |
| S1PR1 | Sphingosine-1-Phosphate Receptor 1 | 0.8 | (2E−03) | −0.23 | (1E−04) | 0.83 | (6E−10) | −0.32 | (2E−45) |
| TUBB2A | Tubulin Beta 2A Class IIa | 0.77 | (3E−02) | −0.18 | (3E−03) | 0.81 | (2E−02) | −0.16 | (2E−11) |
| ABLIM1 | Actin Binding LIM Protein 1 | 0.61 | (6E−10) | −0.52 | (1E−19) | 0.81 | (8E−12) | −0.31 | (2E−42) |
| MYC | MYC Proto-Oncogene, BHLH | 0.53 | (1E−09) | −0.53 | (2E−20) | 0.81 | (9E−14) | −0.46 | (8E−97) |
| | Transcription Factor | | | | | | | | |

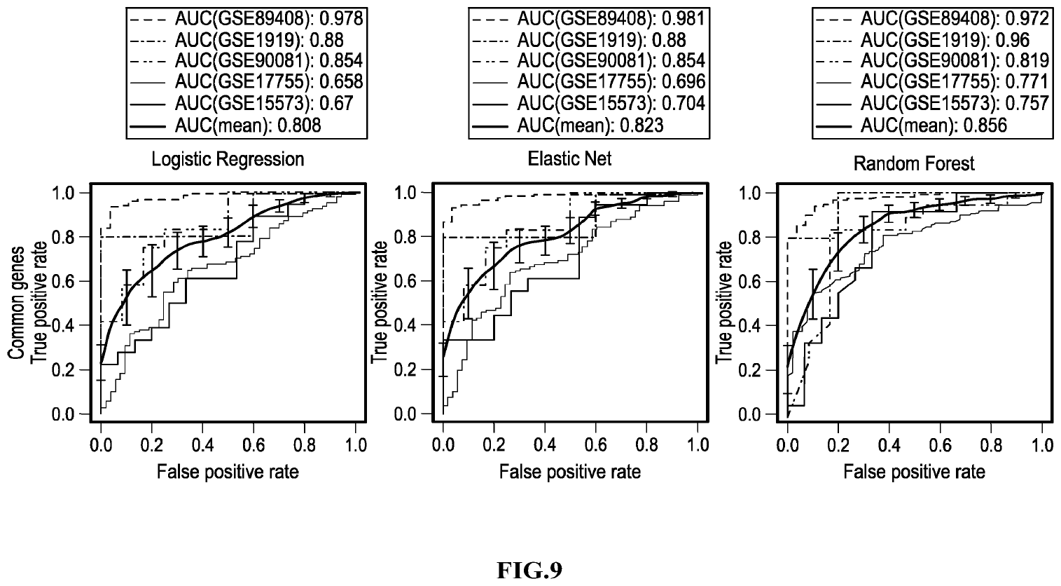

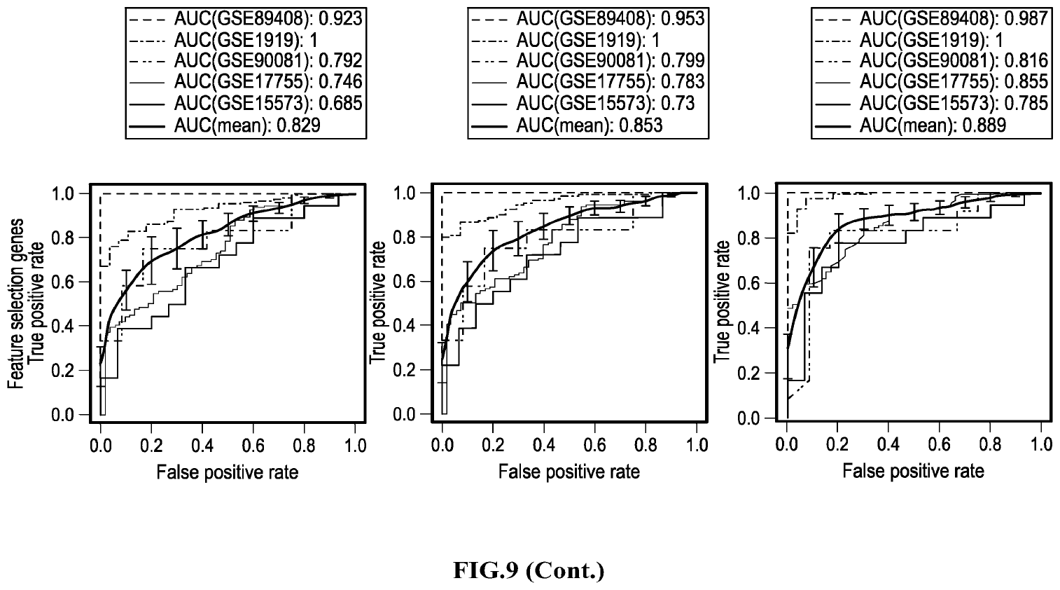

For validation purposes, we leveraged 5 publicly available independent datasets on synovium and blood (see Methods) (Table 1). Since not all genes were measured across the studies, the set was reduced to 25 common DE genes and 38 feature selected genes. We found the set of feature selected genes has superior performance over the set of common DE genes for all three ML methods (FIG. 9). The largest difference in performance was for the Random Forest model: the model with the common DE genes had an AUC of 0.856±0.046 (95% CI (0.775, 0.937)) (FIG. 4B), while the model with the feature selected genes performed with 0.889±0.044 (95% CI (0.811, 0.966)) (FIG. 4C).

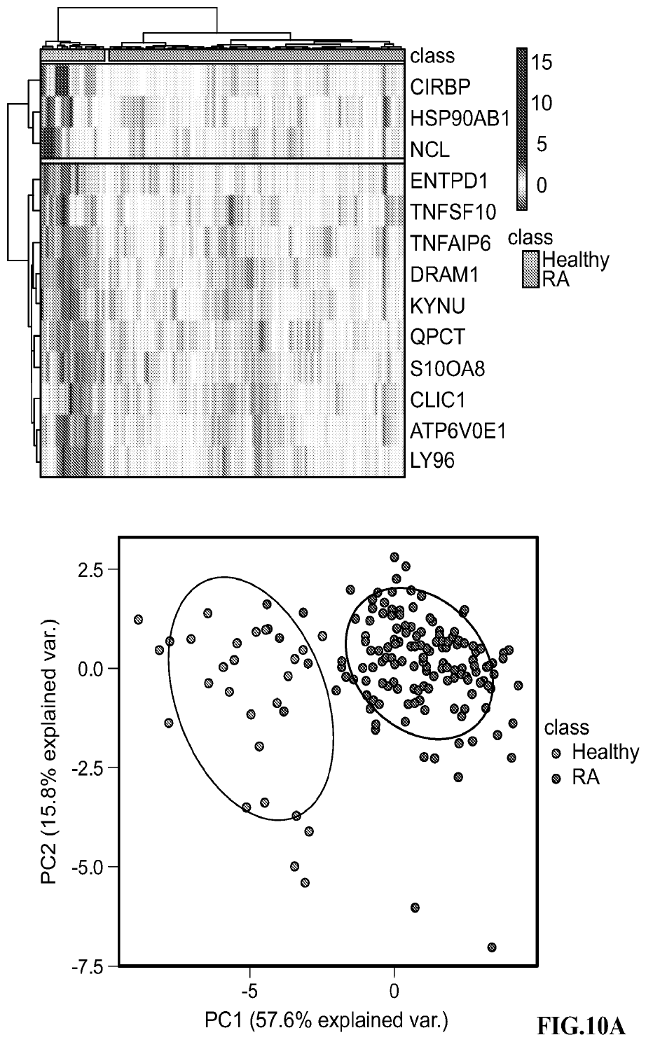

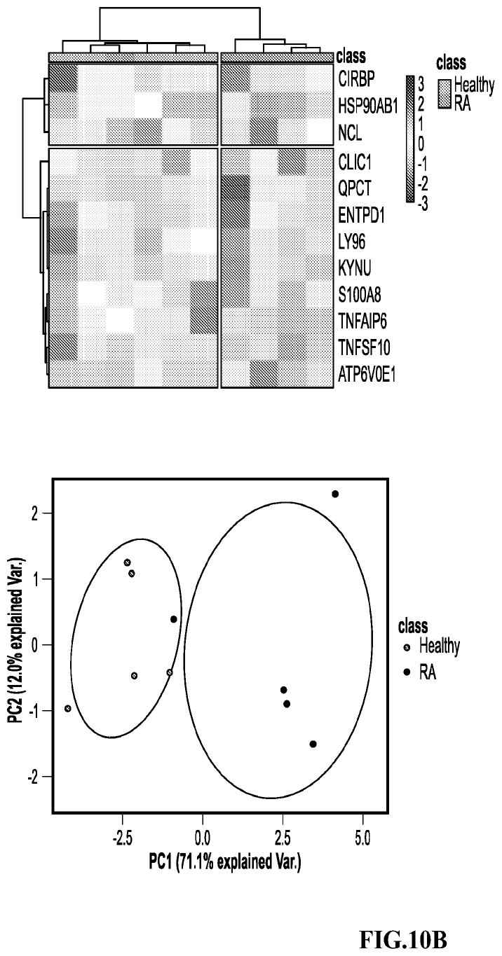

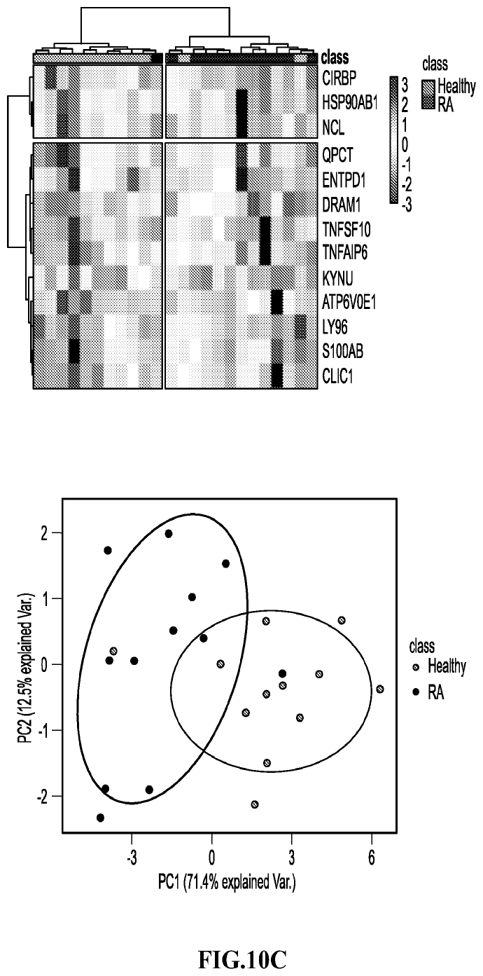

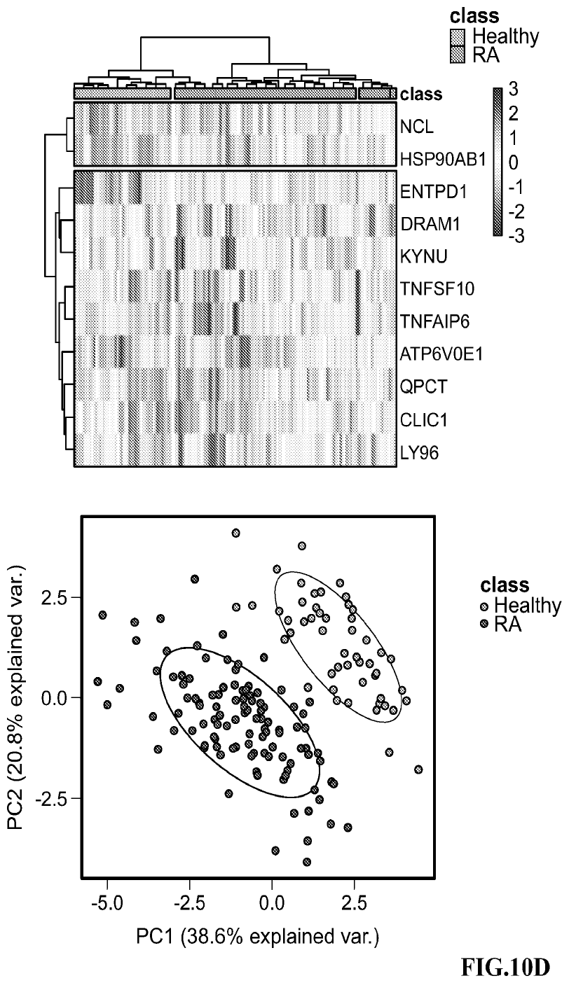

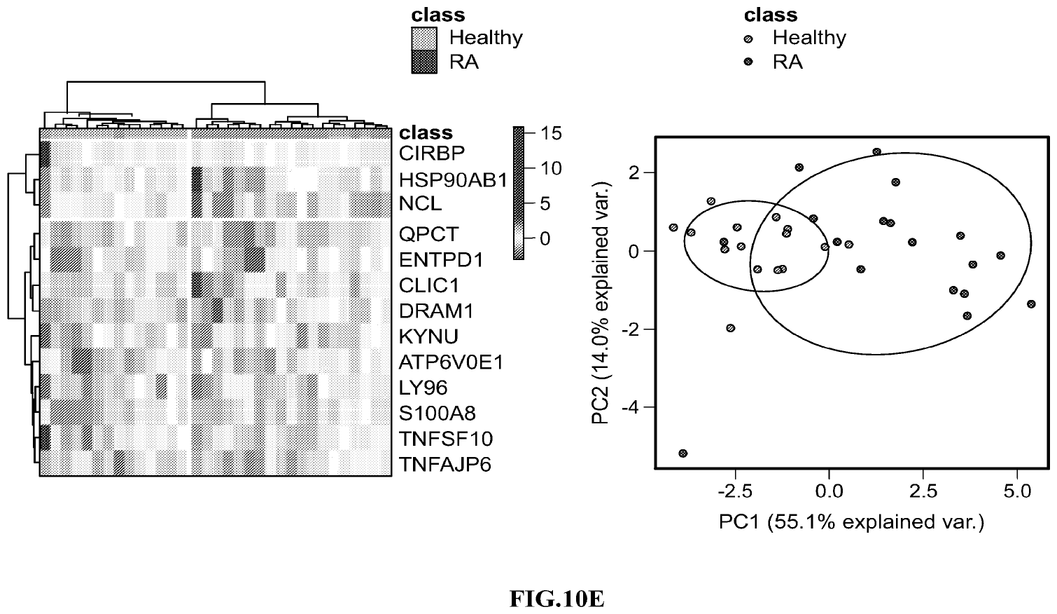

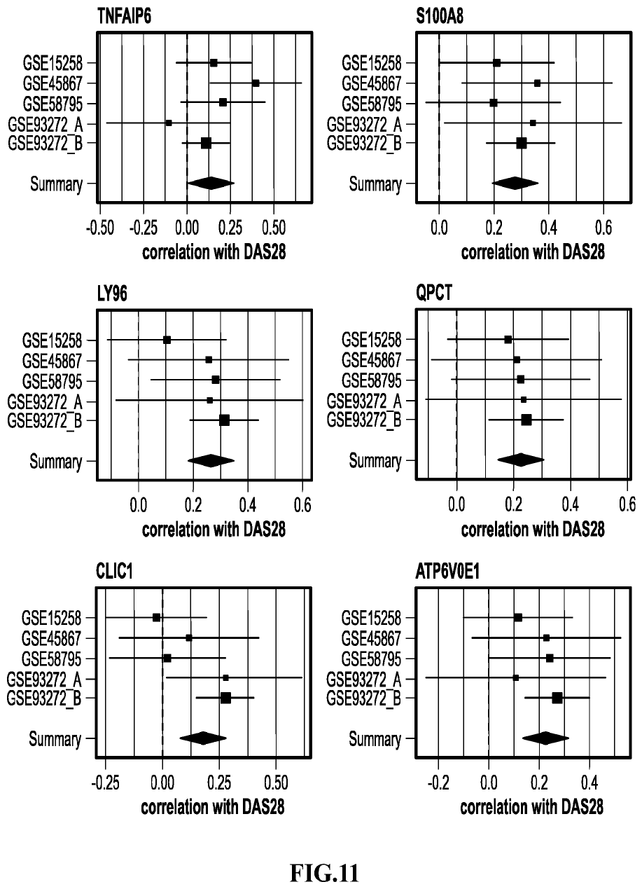

The set of 53 feature selected genes was thresholded with averaged AUC 0.8 using validation sets resulting in the set of 10 up-regulated TNFAIP6, S100A8, TNFSF10, DRAM1, LY96, QPCT, KYNU, ENTPD1, CLIC1, ATP6V0E1, and 3 down-regulated HSP90AB1, NCL, CIRBP genes (FIG. 4A, FIG. 10, Table 6).

TABLE 6
Summary of 13 validated feature selected genes.

| | | | | Synovium | | | Blood | | |
|---|---|---|---|---|---|---|---|---|---|
| | | | FC (BH | ρ (BH | | FC (BH | ρ (BH | | |
| | | | adj, p- | adj. p- | | adj. p- | adj. p- | | Validation |
| Gene | Description | Regulation | value) | value) | AUC | value) | value) | AUC | AUC |
| TNFAIP6 | TNF Alpha | up | 2.46 (4E−06) | 0.39 (7E−11) | 0.81 | 1.36 (BE−16) | 0.39 (3E−67) | 0.77 | 0.88 |
| | induced | | | | | | | | |
| | Protein 6 | | | | | | | | |
| | S100 | | | | | | | | |
| S100AB | Calcium | up | 2.28 (7E−05) | 0.34 (1E−08) | 0.81 | 1.46 (7E−32) | 0.48 (9E−108) | 0.81 | 0.94 |
| | Binding | | | | | | | | |
| | Protein AB | | | | | | | | |
| DRAM1 | DNA | up | 1.55 (6E−07) | 0.46 (3E−15) | 0.93 | 1.18 (8E−15) | 0.41 (6E−76) | 0.79 | 0.81 |
| | Damage | | | | | | | | |
| | Requlated | | | | | | | | |
| | Autophagy | | | | | | | | |
| | Modulator 1 | | | | | | | | |
| | TNF | | | | | | | | |
| TNFSF10 | Superfamily | | 1.55 (1E−09) | 0.52 (3E−19) | 0.9 | 1.27 (1E−23) | 0.44 (4E−88) | 0.8 | 0.84 |
| | Member 10 | | | | | | | | |
| LY96 | Lymphocyle | up | 1.54 (1E−09) | 0.51 (2E−18) | 0.94 | 1.22 (7E−11) | 0.28 (2E−35) | 0.69 | 0.87 |
| | Antigen 96 | | | | | | | | |
| | Glutaminyi- | | | | | | | | |
| | Peptide | | | | | | | | |
| QPCT | Cyclotransferase | up | 1.46 (4E−05) | 0.39 (7E−11) | 0.92 | 1.19 (4E−10) | 0.29 (1E−37) | 0.71 | 0.82 |
| KYNU | Kynureninase | up | 1.41 (5E−05) | 0.36 (1E−09) | 0.84 | 1.17 (2E−11) | 0.28 (3E−34) | 0.69 | 0.82 |
| ENTPD1 | Ectonucieoside | | 1.33 (1E−08) | 0.52 (2E−19) | 0.94 | 1.21 (2E−16) | 0.4 (5E−71) | 0.78 | 0.86 |
| | Triphosphate | | | | | | | | |
| | Diphosphohy | | | | | | | | |
| | drolase 1 | | | | | | | | |
| | Chloride | | | | | | | | |
| CLIC1 | Intracellular | up | 1.32 (5E−08) | 0.47 (7E−16) | 0.91 | 1.2 (5E−27) | 0.47 (4E−103) | 0.84 | 0.8 |
| | Channel 1 | | | | | | | | |
| | ATPase H+ | | | | | | | | |
| ATP6V0E1 | Transporting | up | 1.23 (3E−04) | 0.37 (8E−10) | 0.84 | 1.08 (4E−10) | 0.28 (3E−35) | 0.7 | 0.82 |
| | V0 Subunit | | | | | | | | |
| NCL | Nacleolin | down | 0.83 (2E−05) | −0.39 (4E−11) | 0.82 | 0.88 (4E−09) | −0.32 (2E−44) | 0.72 | 0.82 |
| | Coid | | | | | | | | |
| | inducible | | | | | | | | |
| CIRBP | | down | 0.8 (3E−05) | −0.41 (4E−12) | 0.83 | 0.91 (2E−10) | −0.33 (2E−47) | 0.74 | 0.89 |
| | RNA Binding | | | | | | | | |
| | Protein | | | | | | | | |
| | Heat Shock | | | | | | | | |
| | Protein 90 | | | | | | | | |
| HSP90AB1 | Alpha Famiy | down | 0.79 (2E−04) | −0.37 (3E−10) | 0.82 | 0.84 (4E−12) | −0.36 (7E−56) | 0.73 | 0.8 |
| | Class B | | | | | | | | |
| | Member 1 | | | | | | | | |

iv. Clinical Implications of Transcription Based Disease Score

In order to assess the clinical utility of the feature selected genes, we introduced a scoring function, RAScore, which is derived by subtracting the geometric mean of expression values of down-regulated genes from the geometric mean of up-regulated genes. With this definition, the RAScore is 2-fold (95% CI (1.8, 2.2), p=3e-15) larger for RA in comparison to Healthy samples in synovium. In whole blood, the RAScore has an effect size of 1.37 (95% CI (1.34, 1.4), p=1e-108). On the validation synovium data, the RAScore had a mean effect size 5.5 (95% CI (3.8, 8.2), p=1e-10) and 2.4 (95% CI (2.1, 2.8), p=3e-23) on the validation blood data.

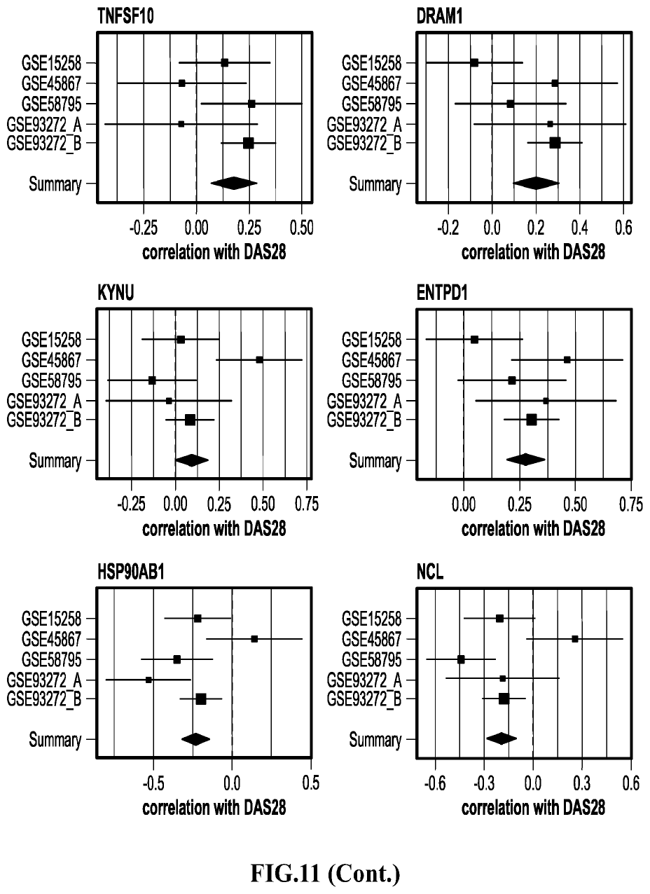

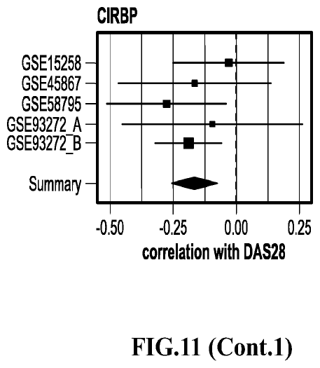

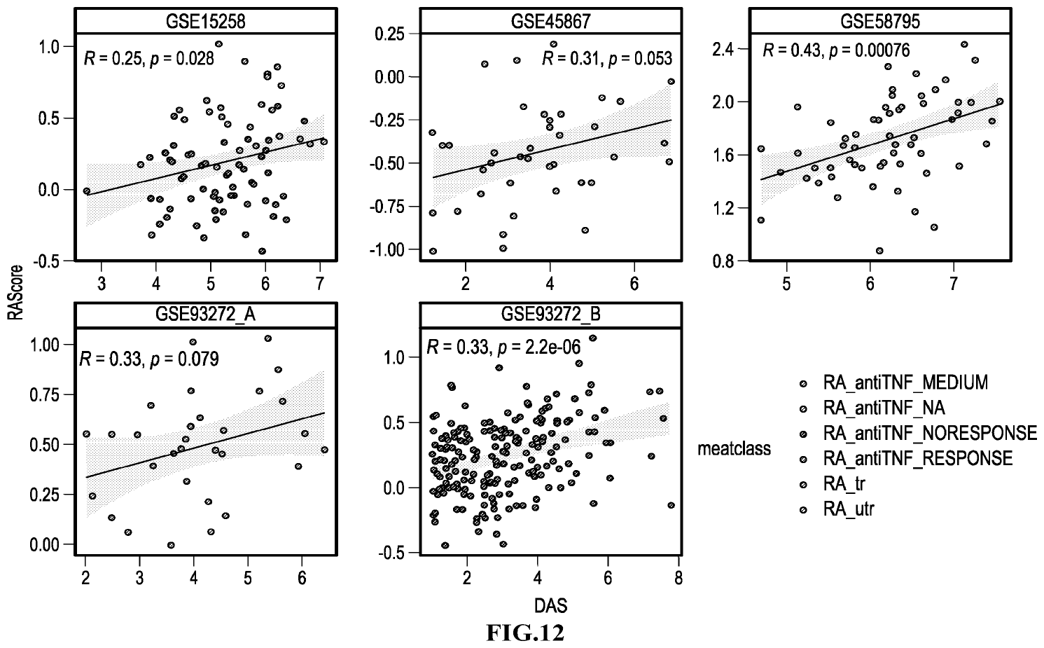

We identified 4 datasets with 411 samples with available disease activity score (DAS28) annotations. To determine if the feature selected genes were associated with DAS28, and thus potentially useful as a disease activity biomarker, we assessed the correlation of the expression value of each gene with the DAS28 score. The RAScore was overall positively correlated with DAS28 with the most correlated gene being S100A8 with mean r=0.28 (95% CI [0.19, 0.37]) and most anti-correlated gene HSP90AB1 with mean r=−0.23 (95% CI [−0.32, −0.14]) (FIG. 5B, FIG. 11). We also determined the correlation of the RAScore with DAS28 in these datasets and obtained Pearson correlation coefficient from 0.25 to 0.43 in blood and 0.31 in synovium (FIG. 12). The average correlation was 0.33 with 95% CI [0.24, 0.41] (FIG. 5A).

To investigate the ability of the RAScore to differentiate RA from osteoarthritis (OA), we identified 6 datasets that had both RA and OA samples available. FIG. 5E shows the distributions of RAScore for RA, OA, and Healthy samples in 6 available datasets. In most datasets, the RAScore was able to significantly differentiate OA from RA and Healthy samples (p=2.3e-6) implicating that this score may be useful diagnostically.

The RAScore performed similarly in both RF-positive and RF-negative rheumatoid arthritis samples in the whole blood dataset GSE74143 suggesting the applications of this score are generalizable to these RA subtypes (p=0.9) (FIG. 5C). Furthermore, we tested the utility of this score in datasets from polyarticular juvenile idiopathic arthritis (JIA) samples given that this subtype of JIA is most similar to RA, and also found good performance in the ability to differentiate JIA from healthy controls (OR 1.29, 95% CI [1.00, 1.57], p=2e-4) (FIG. 5F). Thus, this score may also be useful in the pediatric arthritis population.

Lastly, it appears the RA score also tracks with treatment response. In 2 datasets, RA patients had transcriptional measurements before and after treatment with DMARD. The RA score significantly (p=2e-4) decreases between pre- and post-treatment measurements (FIG. 5D).

3. Discussion

In this study, we leveraged publicly available microarray gene expression data from both synovium and peripheral blood tissues in search of putative biomarkers for Rheumatoid Arthritis (RA). We first applied a conventional approach (ref to prev. studies on biomarkers) of intersecting the differentially expressed (DE) genes from both tissues and obtained a list of 32 common genes. Our results showed that agreement with previous findings. Pathway analysis of these genes showed their involvement in similar biological processes that were found and described before. The common DE genes having a higher expression in both tissues formed denser and more distinct clusters of both RA and control samples in synovium (FIG. 2E, FIG. 2F) and blood (FIG. 2G, FIG. 2H), unlike all DE genes (FIG. 7A, FIG. 7B, FIG. 8A and FIG. 8B). However, there are some limitations to this kind of approach that should be recognized. The list of common DE genes is limited by a chosen threshold for a fold change. Genes that are still important in association with the disease and could potentially be biomarkers but have fold changes even slightly below our threshold are filtered out. Another caveat is that there are a number of highly co-expressed genes in the list and, from a computational perspective, it is not clear which one would be a better performing biomarker. Some prioritization approach to shorten the list of highly co-expressed genes is required here.

In order to identify a robust and non-redundant set of biomarkers, we developed a specific feature selection pipeline that leveraged the data from both tissues in concordance and was based on statistical analysis and machine learning techniques. This resulted in 53 protein coding genes that outperformed 32 common genes in outcome prediction tasks on independent data. In further validation steps, we identified and selected 10 up-regulated and 3 down-regulated genes with the highest performance. The up-regulated genes are highly expressed in diseased synovial tissue, and their elevated protein levels in blood can be the direct markers for RA disease.

We went further in combining the 13 feature selected genes into a transcriptional gene score, RAScore, that potentially could serve as a clinical tool in a blood test for early RA recognition and monitoring disease progression (FIG. 5A). Moreover, the RAscore was able to significantly discriminate RA from OA (another most common but non-inflammatory arthritis type) giving this even more potential clinical value (FIG. 5E). RAScore did not differentiate between RF+ and RF− sub-types of RA (FIG. 5C) based on one available dataset, suggesting the generalizability of this metric. The pediatric arthritis closest to RA, polyarticular Juvenile Idiopathic Arthritis (polyJIA), was also recognized by RAScore (FIG. 5F) in blood. Some genes/proteins from the score were previously found to be associated with JIA. The effect of the treatment was also captured with significantly lower RAScore for DMARD treated patients in comparison to treatment-naive ones (FIG. 5D).

The 13 genes identified using these machine learning methods represent candidate biomarkers in RA. These biomarkers provide insight into RA pathogenesis and could represent treatment targets, disease activity biomarkers or predictors of flare, to be explored in future studies. There is evidence to support a role in RA for a few of these genes, while others are novel findings.

The gene TNFAIP6, also known as TSG-6, encodes for a secretory protein that contains a hyaluronan-binding domain involved with extracellular matrix stability and cell migration. This protein is not a constituent of healthy adult tissues but produced in response to inflammatory mediators, with high levels detected in the synovial fluid of patients with rheumatoid arthritis. TNFAIP6 is thought to affect the destruction of inflammatory tissue through its role in extracellular matrix remodeling.

In this study we presented a robust pipeline of search for putative biomarkers: each gene went individually through a feature selection procedure with multiple iterations on the discovery data and was independently tested on the validation cohorts. The gene redundancy was decreased selecting the most performing genes in RA association prediction. The strength of RAScore is in the independence of its composing genes. Even though one or more newly discovered biomarkers fail in an experiment, the RAScore will still work with the rest of genes.

However, some limitations are present in this study. The data was collected from the public repository NCBI GEO where often the case-control ratio was highly imbalanced up to a full absence of healthy controls especially in whole blood. We separately collected two datasets of healthy individuals to enrich the blood data with the control class. All sample annotations were kept from the original publications, though for 40% of samples the sex annotations were not available, and they were imputed based on the expression levels of Y chromosome genes.

Another limitation to the study results were the limited availability of validation cohorts that would have a fair case-control balance. Out of three validation blood datasets, two were from PBMC in contrast to the whole blood discovery data. This could possibly lead to lower AUC in gene performances on the validation datasets, that is to lower gene filtration rate overall.

Additionally, the most case samples were from RA patients with various medications. Even though the treatments were used in the DGE analysis as covariates (including untreated patients) there still exists the possibility of their impact on the results.

The further development of the RAScore as a clinical tool requires the validation of its composing genes with experimental analysis of the protein levels in RA patients and healthy individuals. A potential longitudinal study would bring better understanding of the diagnostic and disease monitoring capability of the tool.

1. A method of selecting a biomarker associated with a disorder or disease, the method comprising:
a) creating a test data set and a training data set from an input set of data, wherein the input set of data comprises gene expression profiles of subjects having the disorder or disease and control subjects;
b) identifying one or a plurality of significant expression profiles correlated with the disorder or disease in the training data set using a statistical test;
c) evaluating expression performance of each of the significant expression profiles by applying one or a plurality of machine learning methods to create a performance algorithm;
d) testing the performance algorithm on the test data set;
e) selecting a high performing expression profile corresponding to at least one biomarker based upon a first threshold of the performance algorithm;
f) testing the high performing expression profile selected in step e) with a dataset, said dataset being independent from the input set of data; and
g) selecting a biomarker associated with the disorder or disease based on a second threshold of the performance algorithm.

2. (canceled)

3. The method of claim 1, further comprising one or a combination of: (i) compiling data from a provider; (ii) assessing quality control; and/or (iii) data processing normalizing prior to performing step a).

4. The method of claim 1, wherein the test data set and the training data set comprise a random spilt of the input set of data in a ratio of about 1:3, 1:4 or 1:5.

5. The method of claim 1, wherein the statistical test used in step b) to identify the set of significant expression profiles comprises linear models for microarray data (limma) with a p-value less than about 0.05.

6-7. (canceled)

8. The method of claim 1, wherein the performance algorithm is validated on the test data set using area under receiver operating characteristic (AUROC) curve wherein the AUROC is from about 0.5 to about 0.9.

9.-15. (canceled)

16. The method of claim 1, further comprising eliminating an expression profile of a particular gene, locus or nucleic acid sequence from being a biomarker if the expression profile performance of said particular gene, locus or nucleic acid sequence is inconsistent between different tissue types.

17.-19. (canceled)

20. A composition comprising nucleic acid sequences complementary to one or a combination of: TNFAIP6, S100A8, TNFSF10, DRAM1, LY96, QPCT, KYNU, ENTPD1, CLIC1, ATP6V0E1, HSP90AB1, NCL, and CIRBP.

21. The composition of claim 20, wherein:
a) the nucleic acid sequence is complementary to a nucleic acid sequence comprising at least about 70% sequence identity to SEQ ID NO: 1;
b) the nucleic acid sequence is complementary to a nucleic acid sequence comprising at least about 70% sequence identity to SEQ ID NO: 3, SEQ ID NO: 5, SEQ ID NO: 7, SEQ ID NO: 9, and/or SEQ ID NO: 11;
c) the nucleic acid sequence is complementary to a nucleic acid sequence comprising at least about 70% sequence identity to SEQ ID NO: 13;
d) the nucleic acid sequence is complementary to a nucleic acid sequence comprising at least about 70% sequence identity to SEQ ID NO: 15, SEQ ID NO: 17 and/or SEQ ID NO: 19;
e) the nucleic acid sequence is complementary to a nucleic acid sequence comprising at least about 70% sequence identity to SEQ ID NO: 21 and/or SEQ ID NO: 23;
f) the nucleic acid sequence is complementary to a nucleic acid sequence comprising at least about 70% sequence identity to SEQ ID NO: 25;
g) the nucleic acid sequence is complementary to a nucleic acid sequence comprising at least about 70% sequence identity to SEQ ID NO: 27, SEQ ID NO: 29 and/or SEQ ID NO: 31;
h) the nucleic acid sequence is complementary to a nucleic acid sequence comprising at least about 70% sequence identity to SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47 and/or SEQ ID NO: 49;
i) the nucleic acid sequence is complementary to a nucleic acid sequence comprising at least about 70% sequence identity to SEQ ID NO: 51, SEQ ID NO: 53, and/or SEQ ID NO: 55;
j) the nucleic acid sequence is complementary to a nucleic acid sequence comprising at least about 70% sequence identity to SEQ ID NO: 57;
k) the nucleic acid sequence is complementary to a nucleic acid sequence comprising at least about 70% sequence identity to SEQ ID NO: 59;
l) the nucleic acid sequence is complementary to a nucleic acid sequence comprising at least about 70% sequence identity to SEQ ID NO: 61, SEQ ID NO: 63 and/or SEQ ID NO: 65; and
m) the nucleic acid sequence is complementary to a nucleic acid sequence comprising at least about 70% sequence identity to SEQ ID NO: 67, SEQ ID NO: 69, SEQ ID NO: 71, SEQ ID NO: 73, SEQ ID NO: 75 and/or SEQ ID NO: 77.

22.-26. (canceled)

27. A method of diagnosing a subject with arthritis, the method comprising:
i) detecting the presence, absence and/or quantity of one or a plurality of biomarkers chosen from:
a) a polypeptide comprising at least about 70% sequence identity to SEQ ID NO: 2;
b) a polypeptide comprising at least about 70% sequence identity to SEQ ID NO: 4, SEQ ID NO: 6, SEQ ID NO: 8, SEQ ID NO: 10 and/or SEQ ID NO: 12;
c) a polypeptide comprising at least about 70% sequence identity to SEQ ID NO: 14;
d) a polypeptide comprising at least about 70% sequence identity to SEQ ID NO: 16, SEQ ID NO: 18 and/or SEQ ID NO: 20;
e) a polypeptide comprising at least about 70% sequence identity to SEQ ID NO: 22 and/or SEQ ID NO: 24;
f) a polypeptide comprising at least about 70% sequence identity to SEQ ID NO: 26;
g) a polypeptide comprising at least about 70% sequence identity to SEQ ID NO: 28, SEQ ID NO: 30 and/or SEQ ID NO: 32;
h) a polypeptide comprising at least about 70% sequence identity to SEQ ID NO: 34, SEQ ID NO: 36, SEQ ID NO. 38, SEQ ID NO: 40, SEQ ID NO: 42, SEQ ID NO: 44, SEQ ID NO: 46, SEQ ID NO: 48 and/or SEQ ID NO: 50;
i) a polypeptide comprising at least about 70% sequence identity to SEQ ID NO: 52, SEQ ID NO: 54 and/or SEQ ID NO: 56;
j) a polypeptide comprising at least about 70% sequence identity to SEQ ID NO: 58;
k) a polypeptide comprising at least about 70% sequence identity to SEQ ID NO: 60;
l) a polypeptide comprising at least about 70% sequence identity to SEQ ID NO: 62, SEQ ID NO: 64 and/or SEQ ID NO: 66; and
m) a polypeptide comprising at least about 70% sequence identity to SEQ ID NO: 68, SEQ ID NO: 70, SEQ ID NO: 72, SEQ ID NO: 74, SEQ ID NO: 76 and/or SEQ ID NO: 78.

28. The method of claim 27, further comprising obtaining a sample from the subject.

29. The method of claim 28, wherein the sample is blood and/or synovium.

30. The method of claim 27, further comprising:
ii) calculating a geometric mean expression of up-regulated biomarkers chosen from a) through j);
iii) calculating a geometric mean expression of down-regulated biomarkers chosen from k) through m); and
iv) calculating a rheumatoid arthritis score (RAScore) by subtracting the geometric mean expression of the down-regulated biomarkers from the geometric mean expression of the up-regulated biomarkers.

31. The method of claim 27, further comprising a step of diagnosing the subject as having arthritis if the presence, absence and/or quantity of one or a plurality of the biomarkers chosen from a) through m) are at a biologically significant level or levels.

32. The method of claim 30, further comprising a step of diagnosing the subject as having or not having rheumatoid arthritis if the presence, absence and/or quantity of one or a plurality of the biomarkers chosen from a) through in) are at a biologically significant level or levels based at least on the RAScore.

33.-45. (canceled)

46. A computer program product encoded on a computer-readable storage medium comprising instructions for:
a) creating a test data set and a training data set from the input set of data, wherein the input set of data comprises gene expression profiles of subjects having the disorder or disease and control subjects;
b) identifying one or a plurality of significant expression profiles correlated with the disorder or disease in the training data set using a statistical test;
c) evaluating expression performance of each of the significant expression profiles by applying one or a plurality of machine learning methods to create a performance algorithm;
d) testing the performance algorithm on the test data set;
e) selecting a high performing expression profile corresponding to at least one biomarker based upon a first threshold of the performance algorithm;
f) testing the high performing expression profile selected in step e) with a dataset, said dataset being independent from the input set of data; and
g) selecting a biomarker associated with the disorder or disease based on a second threshold of the performance algorithm.

47.-49. (canceled)

50. The computer program product of claim 46, wherein the statistical test used in step b) comprises linear models for microarray data (limma) with a p-value less than about 0.05.

51. The computer program product of claim 46, wherein the one or plurality of machine learning methods used in step c) comprise a linear regression, a logistic regression, a decision tree, an elastic net and/or a random forest.

52. The computer program product of claim 46, wherein the one or plurality of machine learning methods used in step c) comprise a logistic regression model.

53. The computer program product of claim 46, wherein performance algorithm is validated on the test data set using area under receiver operating characteristic (AUROC) curve; wherein the first threshold is a mean AUROC higher than about 0.6 and wherein the second threshold is a mean AUROC is equal to or higher than about 0.8.

54.-59. (canceled)

60. The computer program product of claim 46, wherein the input set of data comprises expression profiles from different tissue types.

61. The computer program product of claim 60, further comprising an instruction for eliminating an expression profile of a particular gene, locus or nucleic acid sequence from being a biomarker if the expression profile performance of sad particular gene, locus or nucleic acid sequence is inconsistent as between different tissue types.

62. A system comprising:
a) the computer program product of claim 46 and
b) a processor operable to execute programs; and/or a memory associated with the processor.

63. A system for selecting a biomarker associated with a disorder or disease, the system comprising:
a processor operable to execute programs;
a memory associated with the processor;
a database associated with said processor and said memory; and
a program product stored in the memory and executable by the processor, the program being operable for:
a) creating a test data set and a training data set from the input set of data, wherein the input set of data comprises gene expression profiles of subjects having the disorder or disease and control subjects;
b) identifying one or a plurality of significant expression profiles correlated with the disorder or disease in the training data set using a statistical test;
c) evaluating expression performance of each of the significant expression profiles by applying one or a plurality of machine learning methods to create a performance algorithm;
d) testing the performance algorithm on the test data set;
e) selecting a high performing expression profile corresponding to at least one biomarker based upon a first threshold of the performance algorithm;
f) testing the high performing expression profile selected in step e) with a dataset, said dataset being independent from the input set of data; and
g) selecting a biomarker associated with the disorder or disease based on a second threshold of the performance algorithm.

64.-78. (canceled)
